(19) United States
(12) Patent Application Publication
(10) Pub. No.: US 2026/0265772 A1
(43) Pub. Date: Sep. 10, 2026

(54) CIRCULAR RNA AND PREPARATION METHOD THEREOF

(71) Applicant: RIBOX THERAPEUTICS HK LIMITED, Hong Kong (CN)

(72) Inventors: Siqi LI, Shanghai (CN); Yunni LEI, Shanghai (CN); Xiaoliang SHI, Shanghai (CN); Weiyi ZHANG, Shanghai (CN)

(21) Appl. No.: 19/166,896

(22) PCT Filed: Mar. 21, 2024
(86) PCT No.: PCT/CN2024/082941

§ 371 Date: Sep. 19, 2025

(30) Foreign Application Priority Data
Mar. 21, 2023 (WO) PCT/CN2023/082833
Jun. 9, 2023 (WO) PCT/CN2023/099386

(51) Int. Cl. C12N 15/85; A61K 48/00; C12N 15/67

(52) U.S. Cl. CPC C12N 15/85; A61K 48/0066; C12N 15/67; C12N 2840/44; C12N 2840/60

(57) ABSTRACT

The present invention relates to the field of biomedicine, in particular, to an engineered circular RNA molecule, a pharmaceutical composition comprising the same, and use thereof. The present invention also relates to the vector as well as the method used for preparing the same.

### TECHNICAL FIELD

The present invention relates to the field of biomedicine, in particular, to an engineered circular RNA molecule, a pharmaceutical composition comprising the same, and use thereof. The present invention also relates to the vector as well as the method used for preparing the same.

### BACKGROUND

Circular RNA is a common type of RNA in eukaryotes. Naturally occurring circular RNAs are primarily produced by a molecular mechanism within cells called “back-splicing”. Eukaryotic circular RNAs have been found to have a variety of molecular and cellular regulatory functions. For example, circular RNAs can regulate the expression of target genes by binding to microRNAs. Circular RNAs can also regulate gene expression by directly binding to target proteins.

Due to its circular nature, circular RNA has a longer half-life than linear mRNA, so it is speculated that circular RNA synthesized in vitro may have higher stability. Methods of forming circular RNAs in vitro include the chemical method, the enzymatic catalysis method, and the ribozyme catalysis method. Chemical methods are expensive and the size of the circular RNA molecules that can be produced is limited. The enzymatic method mainly utilizes T4 RNA ligase to catalyze the circularization of linear RNA, and the size of RNA payload that can achieve circularization is also limited. Ribozyme catalysis (e.g., based on Group I introns) is a promising method for the preparation of circular RNAs.

The natural Group I Intron system can undergo cleavage and ligation reactions to form circular intronic RNAs. A specific cleavage site conserved sequence located in the 5′ exon E1 is cleaved by the nucleophilic attack of the free 3′ hydroxyl group of guanosine triphosphate, resulting in a naked 3′ hydroxyl group, while the guanylate binds on the cleaved 5′ exon E1. After that, the exposed 3′ hydroxyl group at the 5′ end of the intron attacks the conserved sequence between the 3′ end of the intron and the exon E2, the exon E2 is excised, and the intron undergoes a circularizing reaction to obtain a circular intronic RNA. An improved ribozyme-catalyzed approach derived from Anabaena tRNA introns has been reported for the formation of circular RNAs in vitro, termed the “Group I permuted intron-exon self-splicing system, PIE system”. This method can excise the intron to form circular RNA containing exons. Therefore, this method has the potential to form expressible circular RNAs. The basic design principle of the PIE system is to connect the exon E1 sequence and E2 sequence end to end by molecular cloning to form a continuous circular plasmid. The intron is cleaved by restriction endonuclease to obtain a linear plasmid. Then, in vitro transcription is performed through the T7 promoter upstream of the permuted 3′ intron to obtain a linear RNA containing a 3′ intron-E2-E1-5′ intron structure. Similar to the natural Group I intron system, the specific cleavage site conserved sequence of exon E1 is cleaved by the nucleophilic attack of the free 3′ hydroxyl of guanylate, and the exon E1 produces a naked 3′ hydroxyl, while the guanylate binds to the cleaved 5′ intron. Subsequently, the exposed 3′ hydroxyl of exon E1 attacks the conserved sequence between the 3′ intron and exon E2, the 3′ intron is excised, and exon E2 and E1 undergo a circularizing reaction to obtain circular E1-E2 RNA.

However, Group I Introns available for in vitro circularization in the prior art are limited and only a few of them have been proved to be useful in the PIE system to form circular RNAs. Therefore, there is a need in the art to develop new Group I Introns which can be used or in vitro RNA circularization with high efficiency.

### SUMMARY OF THE INVENTION

In one aspect, it is an object of the present invention to provide a circular RNA precursor comprising from 5′ to 3′ direction:
a) a 3′ self-splicing intron fragment; and
b) a 5′ self-splicing intron fragment;
wherein the 3′ self-splicing intron fragment and the 5′ self-splicing intron fragment in combination retain the self-splicing activity of a self-splicing intron and allow generation of a circular RNA through the self-splicing of the circular RNA precursor,
wherein the self-splicing intron is selected from the Group I introns and Group II introns, for example, the self-splicing intron is selected from Group I introns,
wherein the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.

In aspects and embodiments of the present invention, the self-splicing intron is selected from the following group: cyanobacterium Anabaena Group I Intron, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At).

In aspects of the present invention, the circular RNA precursor further comprises a homology arm sequence I upstream of the 3′ self-splicing intron fragment and a homology arm sequence II downstream of the 5′ self-splicing intron fragment.

In one aspect, it is an object of the present invention to provide a circular RNA precursor comprising from 5′ to 3′ direction:
a) a 3′ self-splicing intron fragment;
b) a first residual circularizing element;
c) a nucleotide sequence of interest;
d) a second residual circularizing element; and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the first residual circularizing element, the nucleotide sequence of interest, and the second residual circularizing element through the self-splicing of the circular RNA precursor.

In one aspect, it is an object of the present invention to provide a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a circularizing sequence fragment II comprising a ribozyme recognition site II at its 5′ end and a circularizing sequence fragment I comprising a ribozyme recognition site I at its 3′ end,
wherein the circularizing sequence fragment I is derived from a 5′ terminal portion of a circularizing sequence, and the circularizing sequence fragment II is derived from a 3′ terminal portion of a circularizing sequence, and
wherein in the generated circular RNA the ribozyme recognition site I is connected with the ribozyme recognition site II, and thus the circularizing sequence fragment I is connected with the circularizing sequence fragment II to form the circularizing sequence.

In embodiments, the circularizing sequence is a protein-coding sequence, a non-coding sequence, a translation initiation element (TIE), or a non-TIE functional element.

In embodiments, the nucleotide sequence of interest further comprises one or more sequences selected from the following group between the circularizing sequence fragment II and the circularizing sequence fragment I: at least one protein coding sequences, translation initiation element (TIE), a non-TIE functional element, at least one non-coding sequence, or any combination thereof.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

In one aspect, it is an object of the present invention to provide a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is derived from a 5′ terminal portion of at least one protein-coding sequences, and the protein-coding sequence fragment II is derived from a 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence.

In one aspect, it is an object of the present invention to provide a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is the 5′ terminal portion of the protein-coding sequences, and the protein-coding sequence fragment II is the 3′ terminal portion of the protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the protein-coding sequence.

In embodiments, the at least one protein-coding sequence is a sequences encoding a diagnostic or therapeutic protein. In embodiments, the protein encoded by the protein-coding sequence is a mammalian protein, for example a human protein. In embodiments, the at least one protein-coding sequence comprises at least two protein-coding sequences. In embodiments, the at least one protein-coding sequence is a sequence encoding two or more proteins. In embodiments, the at least one protein-coding sequence a sequence encoding two or more diagnostic or therapeutic protein. Such at least two protein-coding sequences are operably linked with each other by spacers, 2A sequence or further translation initiation elements. In embodiments, a further translation initiation element is operably linked between any two adjacent protein-coding sequences. In embodiments, the translation initiation element is selected from an IRES sequence, a 5′ UTR sequence, a Kozak sequence, a sequence containing m⁶A modification, a sequence complementary to ribosome 18S rRNA, or any combination thereof. In embodiments, the translation initiation element is an IRES sequence.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

In one aspect, it is an object of the present invention to provide a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a translation initiation element fragment II, at least one protein-coding sequence, and a translation initiation element fragment I;
wherein the translation initiation element fragment I is derived from a 5′ terminal portion of a translation initiation element, and the translation initiation element fragment II is derived from a 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation.

In one aspect, it is an object of the present invention to provide a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a translation initiation element fragment II, at least one protein-coding sequence, and a translation initiation element fragment I;
wherein the translation initiation element fragment I is the 5′ terminal portion of a translation initiation element, and the translation initiation element fragment II is the 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation.

In embodiments, the at least one protein-coding sequence is a sequence encoding a diagnostic or therapeutic protein. In embodiments, the protein encoded by the protein-coding sequence is a mammalian protein, for example a human protein. In embodiments, the at least one protein-coding sequence comprises at least two protein-coding sequences. In embodiments, the at least one protein-coding sequence is a sequence encoding two or more proteins. In embodiments, the at least one protein-coding sequence a sequence encoding two or more diagnostic or therapeutic protein. Such at least two protein-coding sequences may be operably linked with each other by spacers, 2A sequence or further translation initiation elements. In embodiments, the translation initiation element is selected from an IRES sequence, 5′ UTR sequence, Kozak sequence, a sequence containing m6A, a sequence complementary to ribosome 18S rRNA, or any combination thereof. In embodiments, the translation initiation element is an IRES sequence.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

In one aspect, it is an object of the present invention to provide a circular RNA precursor comprising from 5′ to 3′ direction:
a) a 3′ self-splicing intron fragment;
b) a nucleotide sequence of interest;
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-TIE functional element fragment II, a translation initiation element, and at least one protein-coding sequence, and a non-TIE functional element fragment I;
wherein the non-TIE functional element fragment I is derived from a 5′ terminal portion of a non-TIE functional element, and the non-TIE translation initiation element fragment II is derived from a 3′ terminal portion of the non-TIE functional element, and the non-TIE functional element fragment I comprises a ribozyme recognition site I located at its 3′ end, the non-TIE functional fragment II comprises a ribozyme recognition site II located at its 5′ end, wherein in the generated circular RNA the ribozyme recognition site I is connected with the ribozyme recognition site II, and thus the non-TIE functional element fragment I is connected with the non-TIE functional element fragment II to form the non-TIE functional element.

In embodiments, the non-TIE functional element is a 3′ UTR or a replicon. In embodiments, the at least one protein-coding sequence comprises at least two protein-coding sequences. Such at least two protein-coding sequences may connect with each other by spacers, 2A sequence or further translation initiation elements. In embodiments, the translation initiation element is selected from an IRES sequence, 5′ UTR sequence, Kozak sequence, a sequence containing m⁶A modification, a sequence complementary to ribosome 18S rRNA, or any combination thereof. In embodiments, the translation initiation element is an IRES sequence.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

In one aspect, it is an object of the present invention to provide a circular RNA precursor comprising from 5′ to 3′ direction:
a) a 3′ self-splicing intron fragment;
b) a nucleotide sequence of interest;
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-coding sequence fragment II, and a non-coding sequence fragment I;
wherein the non-coding sequence fragment I is derived from a 5′ terminal portion of a non-coding sequence, and the non-coding sequence fragment II is derived from a 3′ terminal portion of the non-coding sequence, and the non-coding sequence fragment I comprises a ribozyme recognition site I located at its 3′ end, the non-coding sequence fragment II comprises a ribozyme recognition site II located at its 5′ end, wherein in the generated circular RNA the ribozyme recognition site I is connected with the ribozyme recognition site II, and thus the non-coding sequence fragment I is connected with the non-coding sequence fragment II to form the complete non-coding sequence.

In embodiments, the non-coding sequence is an endogenous circular RNA. In embodiments, the nucleotide sequence of interest further comprises one or more non-coding sequences between the non-coding sequence fragment I and the non-coding sequence fragment II.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

In aspects of the present invention, the protein-coding sequence is a sequence encoding an aquaporin protein, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein. In embodiments, the AQP-1 protein is a mammalian AQP-1 protein, in particular, a human AQP-1 protein.

In another aspect, it is an object of the present invention to provide a nucleic acid vector for generating a circular RNA molecule, said vector comprises a coding sequence of the circular RNA precursor as described above.

In another aspect, it is an object of the present invention to provide a circular RNA polynucleotide, which is prepared from the circular RNA precursor as described above or from the nucleic acid vector as described above. In embodiments, the circular RNA polynucleotide of the present invention has a duration of in vivo expression of at least 24 hours (1 day), for example, 36 hours, 48 hours, 72 hours, 96 hours, 5 days, 10 days, 15 days, 16 days, 17 days, 18 days, 19 days, 20 days or more. In another aspect, it is an object of the present invention to provide use of the circular RNA precursor as described above and/or the circular RNA as described above as an expression vector. In embodiments, the expression vector is used to express one or more diagnostic or therapeutic proteins.

In another aspect, it is an object of the present invention to provide a pharmaceutical composition comprising the circular RNA precursor as described above and/or the nucleic acid vector as described above and/or the circular RNA as described above, and a pharmaceutically acceptable carrier.

In another aspect, it is an object of the present invention to provide a method for preparing a circular RNA, comprising:
1) providing the circular RNA precursor as described above or obtaining a circular RNA precursor by transcribing from the nucleic acid vector as described above;
2) incubating the circular RNA precursor in the presence of a divalent metal cation at a temperature at which RNA circularization occurs; and
3) harvesting the circular RNA obtained in step 2).

In another aspect, it is an object of the present invention to provide a method for preparing a circular RNA, comprising
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.

In another aspect, it is an object of the present invention to provide a method for purifying a circular RNA, comprising:
a) contacting a mixture comprising the circular RNA and the circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.

In another aspect, it is an object of the present invention to provide a method for purifying a circular RNA, comprising:
i) contacting a mixture comprising the circular RNA and the circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and optionally, steps i)-iii) are performed multiple times, e.g., 2 times, 3 times, 4 times or more.

In another aspect, it is an object of the present invention to provide a method for purifying a circular RNA, comprising:
i) adding a linear RNA-specific tag to the linear RNA in a mixture comprising circular RNA and linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii),
optionally, steps ii)-iv) are performed multiple times, e.g., 2 times, 3 times, 4 times or more.

In another aspect, it is an object of the present invention to provide a product expressed by the circular RNA as described above, the circular RNA prepared by the method as described above or the circular RNA purified by the method as described above.

In yet another aspect, it is an object of the present invention to provide a pharmaceutical composition comprising the circular RNA precursor as described above and/or the nucleic acid vector as described above and/or the circular RNA as described above and/or the circular RNA prepared by the method as described above and/or the circular RNA purified by the method as described above, and a pharmaceutically acceptable carrier.

In yet another aspect, it is an object of the present invention to provide a method for preventing or treating a disease or condition in a subject, comprising administering to a subject the circular RNA as described above, the circular RNA prepared by the method as described above or the circular RNA purified by the method as described above, or the pharmaceutical composition as described above.

In another aspect, it is an object of the present invention to provide use of the circular RNA as described above, the circular RNA prepared by the method as described above or the circular RNA purified by the method as described above, or the pharmaceutical composition as described above in the manufacture of a medicament for preventing or treating a disease or condition in a subject.

In some aspects, it is an object of the present invention to provide a method of screening a target open reading frame (ORF) sequence encoding a target polypeptide for a nucleotide motif, comprising steps of:
1) extracting amino acid units in the direction of N terminal to C terminal in the target polypeptide containing m amino acids, and each group of amino acid units includes an amino acid with the number of n; with a step of 1 amino acid, slide along the N terminal to the C terminal, n is an integer and n≥2, m is an integer and ≥1;
2) listing all nucleotide sequences corresponding to each group of amino acid units according to their degenerate codons; and
3) identifying nucleotide sequences obtained from step 2) that match the nucleotide motif,
wherein the nucleotide motif consists of any one of SEQ ID NO: 95-116, preferably CUU, CUC or CAU, and
4) optionally, selecting the target ORF as the circularizing sequence of a circular RNA precursor.

In some aspects, it is an object of the present invention to provide a system for screening a target open reading frame (ORF) for a nucleotide motif, wherein the target open reading frame (ORF) is a nucleotide sequence encoding a target polypeptide, and the system comprises:
a) an amino acid extraction module, which is used for extracting amino acid units in the direction of N terminal to C terminal in the target polypeptide containing m amino acids, wherein each group of amino acid units comprise an amino acid with a number of n, with a step of 1 amino acid, sliding along the N terminal to the C terminal, and n is an integer and n≥2, m is an integer and ≥1;
b) a conversion module, which is used for determining the set of codon sequences of each amino acid unit, each set of codon sequences includes all degenerate codon sequences corresponding to each set of amino acid units; and
c) a target codon sequence filtering module, which is used for determining whether the target codon sequence contain a nucleotide sequence that exactly matches the nucleotide motif,
wherein the nucleotide motif consists of any one of SEQ ID NO: 95-116, preferably CUU, CUC or CAU.

In some aspects, it is an object of the present invention to provide a method for screening a target internal ribosome entry site (IRES) for a nucleotide motif, comprising steps of:
1) identifying regions of non-conserved sequences of the target IRES by comparing the target IRES with the other IRES sequences from the same viral subgenus, through multiple sequence alignment tools, for example: Clustal Omega, or MUSCLE;
2) based on the results of multiple sequence alignment, determining a low-structure region of the target IRES through secondary structure prediction software, such as RNAfold, Mfold, RNAstructure or RNAalifold; and
3) editing 0, 1, or 2 bases in the low-structure region in the target IRES to match the nucleotide motif without affecting the translation initiation activity of said IRES;
wherein the nucleotide motif consists of a nucleotide sequence of any one of SEQ ID NO: 95-116, preferably CUU, CUC or CAU.

In some aspects, it is an object of the present invention to provide a system for screening a target internal ribosome entry site (IRES) for a nucleotide motif, wherein the system comprises:
a) a multi-sequence alignment module, which is used for comparing the target IRES with the other IRES sequences from the same viral subgenus to identify regions of non-conservative sequences;
b) a low-structure prediction module, which is used for determining a low-structure region within the target IRES according to the result of the multiple sequence alignments of the target IRES; and
c) a base editing module, which is used for editing the bases at any one or more positions in the region to obtain an edited region matching the nucleotide motif,
wherein the nucleotide motif consists of a nucleotide sequence of any one of SEQ ID NO: 95-116, preferably CUU, CUC or CAU.

In some aspects, it is an object of the present invention to provide a screening method for screening a target non-TIE functional element for the nucleotide motif in ribozyme recognition site I, comprising a step of:
a) editing 0, 1, or 2 bases on the non-TIE functional element so that the non-TIE functional element sequence (such as 3′ UTR or a replicon) comprises an edited region that matches the nucleotide motif whilst maintaining the non-TIE function;
wherein the nucleotide motif consists of a nucleotide sequence of any one of SEQ ID NO: 95-116, preferably CUU, CUC or CAU.

In some aspects, it is an object of the present invention to provide a system for screening a target region sequence in a non-TIE functional element sequence (such as 3′ UTR or a replicon) for a nucleotide motif, wherein the screening system comprises:
a) a base editing module, which is used for editing the bases at any one or more positions in the region to obtain an edited region matching the nucleotide motif,
wherein the nucleotide motif consists of a nucleotide sequence of any one of SEQ ID NO: 95-116, preferably CUU, CUC or CAU.

It is to be understood that each of the method of prevention or treatment embodiments herein can also be formulated as corresponding use type embodiments.

### BRIEF DESCRIPTION OF THE FIGURES

FIG. 1. Different PIE designs of Azoarcus Group I Intron (Azop).

FIG. 2. The circularization efficiency of Azop with different PIEs.

FIG. 3. PIE design of Scytalidium dimidiatum Group I Intron (S.d).

FIG. 4. The circularization efficiency of S.d with different PIEs.

FIG. 5. Different PIE designs of Staphylococcus phage Twort Group I Intron (Twort).

FIG. 6. The circularization efficiency of Twort with different PIEs.

FIG. 7. Different PIE designs of Scytonema hofmanii Group I Intron (Sh).

FIG. 8. The circularization efficiency of Scytonema hofmanii (Sh).

FIG. 9. Circularization efficiency of IL2 with different Group I Introns of the present invention.

FIG. 10. The RNA circularization efficiency of Agrobacterium-tumefaciens Group I Intron (At).

FIG. 11. Homology arm (HA) design: structure prediction of precursors with different HAs.

FIG. 12. The circularization efficiency of AnaX-IL2 with different Homology arms.

FIG. 13. Analysis of the correlation between circularization efficiency and average base MFE of Homology arm.

FIG. 14. Design of CLEAN-PIE on ORF or IRES from PIE.

FIG. 15. Design of CLEAN-PIE on functional element from PIE.

FIG. 16. Sequence and structure of CLEAN-PIE designs for IL2.

FIG. 17. The circularization efficiency of AnaX Clean PIE for IL2.

FIG. 18. Different CLEAN-PIE Designs for circular RNA polynucleotides expressing the AQP1 protein (CLEAN-PIE circAQP1).

FIG. 19. The circularization efficiency, translation and immunogenicity of Clean PIE for circAQP1 (CLEAN-PIE circAQP1).

FIG. 20. The effect of miRNA target sites in circRNA on expression level.

FIG. 21. The effect of UTR on circRNA translation.

FIG. 22. CLEAN-PIE circAQP1 expression in salivary glands in rats.

FIG. 23. Representative images of IHC staining for AQP1 expression in salivary glands.

### DETAILED DESCRIPTION OF THE INVENTION

In the present invention, unless indicated otherwise, the scientific and technological terminologies used herein refer to meanings commonly understood by a person skilled in the art. Also, the terminologies and experimental procedures used herein relating to protein and nucleotide chemistry, molecular biology, cell and tissue cultivation, microbiology, immunology, all belong to terminologies and conventional methods generally used in the art. For example, the standard DNA recombination and molecular cloning technology used herein are well known to a person skilled in the art, and are described in details in the following references: Sambrook, J., Fritsch, Efland Maniatis, T., Molecular Cloning: A Laboratory Manual; Cold Spring Harbor Laboratory Press: Cold Spring Harbor, 1989. In the meantime, in order to better understand the present invention, definitions and explanations for the relevant terminologies are provided below.

As used herein, the singular forms “a,” “an,” and “the” include plural referents unless the context clearly dictates otherwise. It is further noted that the claims may be drafted to exclude any optional element. For example, a circular RNA precursor refers to one or more circular RNA precursors. As such, the terms “a”, “an”, “one or more” and “at last one” can be used interchangeably. For example, the term “at least one” refers to one, two or more. This statement is intended to serve as antecedent basis for use of such exclusive terminology as “solely,” “only” and the like in connection with the recitation of claim elements, or use of a “negative” limitation. Similarly the terms “comprising”, “including” and “having” can be used interchangeably.

As used herein, the term “and/or” encompasses all combinations of items connected by the term, and each combination should be regarded as individually listed herein. For example, “A and/or B” covers “A”, “A and B”, and “B”. For example, “A, B, and/or C” covers “A”, “B”, “C”, “A and B”, “A and C”, “B and C”, and “A and B and C”.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

In the present application, “optional” or “optionally” means that the subsequently described event or circumstance may or may not occur, and that the description includes instances where said event or circumstance occurs and instances in which it does not.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. Although the nucleotide sequences herein may be represented as DNA sequences (comprising T(s)), when referring to RNA, one skilled in the art can readily determine the corresponding RNA sequence (i.e., replacing T with U).

For example, the nucleotide sequence of interest in the present invention can be a non-coding sequence, such as an antisense RNA, aptamer, guide RNA, or non-coding RNA existing in any organism.

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). There are many methods for determining sequence identity. An example of algorithms suitable for determining percent sequence identity is the algorithm used in the Basic Local Alignment Search Tool (hereinafter “BLAST”), see e.g., Altschul et al., J. Mol. Biol. 215:403-410, 1990 and Altschul et al, Nucleic Acids Res., 15:3389-3402, 1997. Software for performing BLAST analysis is publicly available through the National Center for Biotechnology Information (hereafter “NCBI”). Default parameters used to determine sequence identity using software available from NCBI (such as BLASTN for nucleic acid sequences) are described in McGinnis et al. Nucleic Acids Res., 32: W20-W25, 2004.

A polynucleotide sequence (e.g. a RNA polynucleotide sequence or its fragments) “derived from” a designated polynucleotide sequence means the former polynucleotide sequence is originated from the latter. In some embodiments, the polynucleotide sequence which is derived from a particular polynucleotide sequence has a polynucleotide sequence that is identical, essentially identical or homologous to that particular sequence or a fragment thereof. Polynucleotide sequences derived from a particular polynucleotide sequence may be variants of that particular sequence or a fragment thereof. For example, it will be understood by one of ordinary skill in the art that the circular RNA molecules suitable for use herein may be altered such that they vary in sequence from the sequences from which they were derived, while retaining the desirable activity thereof. “Circular RNA precursor” herein refers to a linear RNA molecule capable of forming a covalently linked closed circular RNA molecule, e.g., by self-splicing. The circular RNA precursor may be produced by transcription from a nucleic acid vector comprising a coding sequence of the circular RNA precursor. Alternatively, the circular RNA precursor may also be obtained by chemical synthesis.

In some embodiments, the circular RNA precursor is capable of forming a covalently linked closed circular RNA molecule by self-splicing under the action of the self-splicing intron fragments and the residual circularizing elements.

As used herein, the term “self-splicing intron” refers to an intron having self-splicing ribozyme activity and capable of excising itself and joining two flanking exons. In some embodiments, the splicing is autocatalytic splicing.

“Self-splicing introns” include, but are not limited to, Group I introns and Group II introns. Group I introns contain 14 subgroups, while most of the Group I introns belong to the IC3 subgroup. For example, the Group I intron may be a Group I intron of the cyanobacterium Anabaena belonging to the IC3 subgroup or a Group I intron from a T4 phage Group I intron belonging to the IA2 subgroup or a Group I intron from Azoarcus sp. BH72 belonging to the IC3 subgroup. Additional examples of self-splicing introns useful in the present invention include, but are not limited to, self-splicing introns derived from the following organisms: Enterobacteriophage T4, Bacteriophage Twort, Bacteriophage SPO1, Bacteriophage S3b, Bacillus anthracis, Clostridium botulinum, Tetrahymena thermophila, Dunaliella parva, Pneumocystis carinii, Physarum polycephalum, Anabaena sp. PCC7120, Scytonema hofmanni, Agrobacterium tumefaciens, Synechocystis PCC 6803, Synechococcus elongatus PCC 6301, Neurospora crassa, Candida albicans, Scytalidium cerradiumydiaces, Pediadiaces Chlamydomonas nivalis, Chlorella vulgaris, Amoebidium parasiticum, Neurospora crassa, Emericella nidulans, Saccharomyces cerevisiae, Schizosaccharomyces pombe, Neochloris aquatica, Dunaliella parva, Symkania negevensis, Emericella nidulans. See e.g., Vicens, Q., et al., (2008). Toward predicting self-splicing and protein-facilitated splicing of group I introns. RNA 14: 2013-2029; Tanner, A. M., et al., (1996). Activity and thermostability of the small self-splicing group I intron in the pre-tRNAIIe of the purple bacterium Azoarcus. RNA 2:74-83.

In some embodiments, the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA Met Group I Intron (Sh), or Agrobacterium-tumefaciens Group I Intron (At). For example, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.

In some embodiments, the 3′ self-splicing intron fragment and the 5′ self-splicing intron fragment are derived from a same self-splicing intron. In some embodiments, the 3′ self-splicing intron fragment is derived from or contains a 3′ terminal portion of the self-splicing intron (a native self-splicing intron), and accordingly the 5′ self-splicing intron fragment is derived from or contains a 5′ terminal portion of the self-splicing intron (a native self-splicing intron). In some embodiments, the 3′ self-splicing intron fragment and the 5′ self-splicing intron fragment in combination retain the self-splicing activity of the self-splicing intron (a native self-splicing intron).

In some embodiments, the self-splicing intron is a Group I intron. In some embodiments, the self-splicing intron is a Group I intron of the IA2 or IC3 subgroup, preferably IC3 subgroup.

In some embodiments, the 3′ self-splicing intron fragment is a 3′ Group I intron fragment. In some embodiments, the 5′ self-splicing intron fragment is a 5′ Group I intron fragment.

As used herein, a 3′ self-splicing intron fragment (e.g., a 3′ Group I intron fragment) is a sequence that is at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the 3′ terminal portion of a native self-splicing intron (e.g., a Group I intron). A 5′ self-splicing intron fragment (e.g., a 5′ Group I intron fragment) is a sequence that is at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the 5′ terminal portion of a native self-splicing intron (e.g., a Group I intron).

It is generally believed that in order to achieve circularization, the native Group I intron needs to be split at an internal site to form a so called “Group I permuted intron-exon self-splicing system, PIE system”. An internal split site as used herein refers to a location where the native Group I intron is split into two portions, and the internal split site is selected such that the generated two separate portions of native Group I intron (the 3′ terminal portion and the 5′ terminal portion) together can maintain ribozyme activity necessary for the self-splicing, even after permutation. It is believed that the two separate portions of native Group I intron maintaining the overall conformation of the native Group I intron can maintain ribozyme activity necessary for the self-splicing. As used herein, the internal split site is located between an intron internal split site I and an intron internal split site II, the intron internal split site I refers to the first nucleotide downstream of the internal split site and the intron internal split site II refers to the first nucleotide upstream of the internal split site. As used herein, “PIE” or “PIE method” or “PIE system” refers to the above described “Group I permuted intron-exon self-splicing system, PIE system”.

In some embodiments, the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.

Only a few Group I introns of which the exact internal split site have been determined by a person skilled in the art, e.g., by referenced to Puttaraju M., et al., (1992) Group I permuted intron-exon (PIE) sequences self-splice to produce circular exons; and/or Puttaraju M., et al., (1996) Circular ribozymes generated in Escherichia coli using group I self-splicing permuted intron-exon sequences. For example, for a Group I intron, especially an Anabaena Group I intron, it can usually be split at a specific site in its P6 region to form the PIE system.

The 3′ terminal portion of a native Group I intron may have a length of about 5% to about 95%, such as about 5%, about 10%, about 20%, about 30%, about 40%, about 50%, about 60%, about 70%, about 80%, about 90%, about 95% of the full length of the native Group I intron. Accordingly, the 5′ terminal portion of a native Group I intron may have a length of about 5% to about 90%, such as about 5%, about 10%, about 20%, about 30%, about 40%, about 50%, about 60%, about 70%, about 80%, about 90%, about 95% of the full length of the native Group I intron.

In some embodiments, the combination of the 3′ Group I intron fragment and the 5′ Group I intron fragment substantially has the self-splicing activity of the corresponding native Group I intron.

“Circularization efficiency” as used herein may refer to the ratio of outcome circular RNA to input precursor in a given time period. Alternatively, “Circularization efficiency” as used herein may refer to the ratio of desired circular RNA to linear circRNA precursor or the ratio of circRNA to the sum of circRNA and linear circRNA precursor in the final product in a given time period. The circularizing efficiency can be determined by methods well known in the art, such as those described in Example 2, 4, 6 and 8 of the present application.

In some embodiments, the circularizing efficiency of the circular RNA is increased by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or more.

“Reduced immunogenicity” as used herein may refer to that the circular RNA, upon contacted with cells, elicits a reduced immune response, i.e., an immune response at a level lower than a control circular RNA or control linear RNA. For example, reduced immune response refers to reduced expression of cytokines. The cytokines include, but are not limited to, IFNβ, TNFα, IL6 and/or RIG-I. In some embodiments, the immunogenicity of the circular RNA is reduced by at least about 10%, at least about 20%, at least about 30%, at least about 40%, at least about 50%, at least about 60%, at least about 70%, at least about 80%, at least about 90% or more.

As used herein, “spacer” refers to any contiguous nucleotide sequence that at least does not negatively interfere with the function of the elements connected by it. Generally, if it is desired to avoid the interaction of two near or adjacent elements, a spacer can be inserted between the two elements. The spacer sequences described herein can serve two functions: (1) to facilitate circularization and (2) to facilitate functionality by allowing correct folding of the residual circularizing element and the nucleotide sequence of interest (e.g., IRES). In some embodiments, the spacer is no more than 150, no more than 100, no more than 50, no more than 30, no more than 10, no more than 5, or no more than 3 nucleotides in length. In some embodiments, the spacer is 5 nucleotides in length. In some embodiments, the spacer is 4 nucleotides in length. In some embodiments, the spacer is 3 nucleotides in length. In some embodiments, the first spacer may be absent. In some embodiments, the second spacer may be absent. In some embodiments, the first spacer and the second spacer may be absent.

In one aspect, the present invention provides a circular RNA precursor comprising from 5′ to 3′ direction:
a) a 3′ self-splicing intron fragment; and
b) a 5′ self-splicing intron fragment;
wherein the 3′ self-splicing intron fragment and the 5′ self-splicing intron fragment in combination retain the self-splicing activity of a self-splicing intron and allow generation of a circular RNA through the self-splicing of the circular RNA precursor,
wherein the self-splicing intron is selected from the Group I introns and Group II introns, for example, the self-splicing intron is selected from Group I introns.

In embodiments of the present invention, the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At).

In some embodiments of the present invention, the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.

In some embodiments of the present invention, when the self-splicing intron is cyanobacterium Anabaena Group I Intron such as AnaX, the intron internal split site II and the intron internal split site I are the nucleotide A at position 115 and the nucleotide A at position 116 from 5′ end of the AnaX intron (SEQ ID NO:1) respectively, and when connected form the nucleotide sequence of AA.

In some embodiments of the present invention, when the self-splicing intron is Azoarcus Group I Intron (Azo) (SEQ ID NO:2),
1) the intron internal split site II and the intron internal split site I are the nucleotide A at position 24 and the nucleotide A at position 25 from 5′ end of the Azo intron respectively, and when connected form the nucleotide sequence of AA (P2);
2) the intron internal split site II and the intron internal split site I are the nucleotide A at position 85 and the nucleotide A at position 86 from 5′ end of the Azo intron respectively, and when connected form the nucleotide sequence of AA (P4-5);
3) the intron internal split site II and the intron internal split site I are the nucleotide C at position 71 and the nucleotide C at position 72 from 5′ end of the Azo intron respectively, and when connected form the nucleotide sequence of CC (P5);
4) the intron internal split site II and the intron internal split site I are a nucleotide C at position 108 and the nucleotide U at position 109 from 5′ end of the Azo intron respectively, and when connected form the nucleotide sequence of CU (P6);
5) the intron internal split site II and the intron internal split site I are the nucleotide A at position 154 and the nucleotide A at position 155 from 5′ end of the Azo intron respectively, and when connected form the nucleotide sequence of AA (P8); or
6) the intron internal split site II and the intron internal split site I are the nucleotide C at position 198 and the nucleotide A at position 199 from 5′ end of the Azo intron respectively, and when connected form the nucleotide sequence of CA (P9).

In some embodiments of the present invention, when the self-splicing intron is Scytalidium dimidiatum Group I Intron (Sd) (SEQ ID NO:4),
1) the intron internal split site II and the intron internal split site I are a nucleotide A at position 88 and a nucleotide G at position 89 from 5′ end of the Sd intron respectively, and when connected form a nucleotide sequence of AG (P2);
2) the intron internal split site II and the intron internal split site I are a nucleotide U at position 173 and a nucleotide G at position 174 from 5′ end of the Sd intron respectively, and when connected form a nucleotide sequence of UG (P5);
3) the intron internal split site II and the intron internal split site I are a nucleotide A at position 204 and a nucleotide G at position 205 from 5′ end of the Sd intron respectively, and when connected form a nucleotide sequence of AG (P6);
4) the intron internal split site II and the intron internal split site I are a nucleotide C at position 252 and a nucleotide G at position 253 from 5′ end of the Sd intron respectively, and when connected form a nucleotide sequence of CG (P8); or
5) the intron internal split site II and the intron internal split site I are a nucleotide C at position 370 and a nucleotide G at position 371 from 5′ end of the Sd intron respectively, and when connected form a nucleotide sequence of CG (P9).

In some embodiments of the present invention, when the self-splicing intron is Staphylococcus phage Twort Group I Intron (Twort) (SEQ ID NO:3),
1) the intron internal split site II and the intron internal split site I are a nucleotide U at position 70 and a nucleotide A at position 71 from 5′ end of the Twort intron respectively, and when connected form a nucleotide sequence of UA (P5);
2) the intron internal split site II and the intron internal split site I are a nucleotide U at position 103 and a nucleotide A at position 104 from 5′ end of the Twort intron respectively, and when connected form a nucleotide sequence of UA (P6);
3) the intron internal split site II and the intron internal split site I are a nucleotide A at position 132 and a nucleotide U at position 133 from 5′ end of the Twort intron respectively, and when connected form a nucleotide sequence of AU (P7);
4) the intron internal split site II and the intron internal split site I are a nucleotide U at position 173 and a nucleotide C at position 174 from 5′ end of the Twort intron respectively, and when connected form a nucleotide sequence of UC (P8);
5) the intron internal split site II and the intron internal split site I are a nucleotide A at position 206 and a nucleotide A at position 207 from 5′ end of the Twort intron respectively, and when connected form a nucleotide sequence of AA (P9);
6) the intron internal split site II and the intron internal split site I are a nucleotide G at position 226 and a nucleotide U at position 227 from 5′ end of the Twort intron respectively, and when connected form a nucleotide sequence of GU (P9.1);
7) the intron internal split site II and the intron internal split site I are a nucleotide U at position 23 and a nucleotide A at position 24 from 5′ end of the Twort intron respectively, and when connected form a nucleotide sequence of UA (P2); or
8) the intron internal split site II and the intron internal split site I are a nucleotide A at position 82 and a nucleotide A at position 83 from 5′ end of the Twort intron respectively, and when connected form a nucleotide sequence of AA (P4-5).

In some embodiments of the present invention, when the self-splicing intron is Scytonema-hofmani tRNA fMet group I intron (Sh) (SEQ ID NO:5),
1) the intron internal split site II and the intron internal split site I are a nucleotide A at position 129 and a nucleotide A at position 130 from 5′ end of the Sh intron respectively, and when connected form a nucleotide sequence of AA (P6).
2) the intron internal split site II and the intron internal split site I are a nucleotide A at position 60 and a nucleotide A at position 61 from 5′ end of the Sh intron respectively, and when connected form a nucleotide sequence of AA (P2).
3) the intron internal split site II and the intron internal split site I are a nucleotide A at position 109 and a nucleotide A at position 110 from 5′ end of the Sh intron respectively, and when connected form a nucleotide sequence of AA (P4-5).
4) the intron internal split site II and the intron internal split site I are a nucleotide A at position 213 and a nucleotide A at position 214 from 5′ end of the Sh intron respectively, and when connected form a nucleotide sequence of UA (P9).

In some embodiments of the present invention, when the self-splicing intron is Agrobacterium-tumefaciens group I intron (At) (SEQ ID NO:6), the intron internal split site II and the intron internal split site I are a nucleotide A at position 107 and a nucleotide A at position 108 from 5′ end of the At intron respectively, and when connected form a nucleotide sequence of AA (P6).

In some embodiments of the present invention, the 3′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 3′ terminal portion of the self-splicing intron, and the 5′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 5′ terminal portion of the self-splicing intron.

In some embodiments of the present invention, the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.

In some embodiments of the present invention, the self-splicing intron is AnaX (SEQ ID NO:1), wherein the 3′ self-splicing intron fragment comprises or consists of a nucleotide sequence of SEQ ID NO: 7, and the 5′ self-splicing intron fragment comprises or consists of a nucleotide sequence of SEQ ID NO: 8.

In some embodiments of the present invention, the self-splicing intron is the Azoarcus Group I Intron (Azo), the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.

In some embodiments of the present invention, the self-splicing intron is the Azoarcus Group I Intron (Azo) (SEQ ID NO:2), wherein the 3′ self-splicing intron fragment and the 5′ self-splicing intron fragment respectively are: SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18.

In some embodiments of the present invention, the self-splicing intron is the Scytalidium dimidiatum Group I Intron (Sd) (SEQ ID NO:4), the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.

In some embodiments of the present invention, the self-splicing intron is the Scytalidium dimidiatum Group I Intron (Sd) (SEQ ID NO:4), wherein the 3′ self-splicing intron fragment and the 5′ self-splicing intron fragment respectively are: SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46.

In some embodiments of the present invention, the self-splicing intron is the Staphylococcus phage Twort Group I Intron (Twort) (SEQ ID NO:3), the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.

In some embodiments of the present invention, the self-splicing intron is the Staphylococcus phage Twort Group I Intron (Twort) (SEQ ID NO:3), wherein the 3′ self-splicing intron fragment and the 5′ self-splicing intron fragment respectively are: SEQ ID NO: 21 and SEQ ID NO: 22, SEQ ID NO: 23 and SEQ ID NO: 24 SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36.

In some embodiments of the present invention, the self-splicing intron is the Scytonema-hofmani Trna Met group I intron (Sh) (SEQ ID NO:5), the 3′ self-splicing intron fragment comprises or consists of a nucleotide sequence of SEQ ID NO: 47, 49, 51, 53, or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51, 53, and the 5′ self-splicing intron fragment comprises or consists of a nucleotide sequence of SEQ ID NO: 48, 50, 52, 54, or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52, 54.

In some embodiments of the present invention, the self-splicing intron is the Scytonema-hofmani Trna fMet group I intron (Sh) (SEQ ID NO:5), wherein the 3′ self-splicing intron fragment and the 5′ self-splicing intron fragment respectively are: SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54.

In some embodiments of the present invention, the self-splicing intron is the Agrobacterium-tumefaciens group I intron (At) (SEQ ID NO:6), the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.

In some embodiments of the present invention, the self-splicing intron is the Agrobacterium-tumefaciens group I intron (At) (SEQ ID NO:6), wherein the 3′ self-splicing intron fragment and the 5′ self-splicing intron fragment respectively are: SEQ ID NO: 55 and SEQ ID NO: 56.

In one aspect, the present invention provides a circular RNA precursor comprising from 5′ to 3′ direction:
a) a 3′ self-splicing intron fragment;
b) a first residual circularizing element;
c) optionally, a nucleotide sequence of interest;
d) a second residual circularizing element; and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 2 to about 200 nucleotides.

As used herein, a “residual circularizing element” refers to an exon-derived sequence that is additionally introduced to facilitate the circularization process. The residual circularizing element is a sequence derived from the native exon of the self-splicing intron (the exon flanking the self-splicing intron) and is involved in or required for circularization by the self-splicing intron and participates in circularization together with the self-splicing intron but is retained in the final circular RNA. In particular, the residual circularizing element is capable of being recognized and/or spliced by the self-splicing intron (or a combination of the first self-splicing intron fragment and the second self-splicing intron fragment), and thus is required for circularization.

As used herein, “exon region” in the residual circularizing element is a sequence derived from the native exon of the self-splicing intron (the exon flanking the self-splicing intron) and capable of being recognized and/or spliced by the self-splicing intron (or a combination of the first self-splicing intron fragment and the second self-splicing intron fragment), and thus is required for circularization. An “exon region” can also be referred as a “splicing site sequence” herein.

In some embodiments, the first residual circularizing element comprises or consists of from 5′ to 3′ direction a 3′ exon region and optionally a first spacer. In the circular RNA precursor, the 5′ end of the 3′ exon region is directly connected to the 3′ end of the 3′ self-splicing intron fragment.

In some embodiments, the 3′ exon region is derived from the native 3′ exon of the self-splicing intron (the exon flanking (downstream of) the 3′ end of the self-splicing intron) or a contiguous fragment thereof starting from the 5′ terminal nucleotide.

In some embodiments, the total length of the first residual circularizing element and the second residual circularizing element is no greater than about 500 nucleotides, e.g., no greater than about 200, about 100, about 90, about 80, about 70, about 60, about 50, about 40, about 30, about 20, about 15, about 10, about 5 nucleotides, about 4, nucleotides, about 3 nucleotides, about 2 nucleotides, about 1 nucleotide or 0 nucleotide.

In some embodiments, the total length of the first residual circularizing element and the second residual circularizing element is from about 2 to about 200 nucleotides, or any integer number therebetween. In some embodiments, the total length of the first residual circularizing element and the second residual circularizing element is about 2 to about 30 nucleotides, or any integer number therebetween. In some embodiments, the total length of the first residual circularizing element and the second residual circularizing element is from about 2 to about 20 nucleotides, or any integer number therebetween. In some embodiments, the total length of the first residual circularizing element and the second residual circularizing element is from about 2 to about 15 nucleotides, or any integer number therebetween. In some embodiments, the total length of the first residual circularizing element and the second residual circularizing element is about 2 to about 10 nucleotides, or any integer number therebetween.

In some embodiments, the total length of the first residual circularizing element and the second residual circularizing element is at least 5 nucleotides in length. In some embodiments, the total length of the first residual circularizing element and the second residual circularizing element is at least 10 nucleotides in length. In some embodiments, the total length of the first residual circularizing element and the second residual circularizing element is at least 15 nucleotides in length.

In some embodiments, the total length of the first residual circularizing element and the second residual circularizing element is from about 5 to about 100 nucleotides, or any integer number therebetween. In some embodiments, the total length of the first residual circularizing element and the second residual circularizing element is from about 10 to about 100 nucleotides, or any integer number therebetween. In some embodiments, the total length of the first residual circularizing element and the second residual circularizing element is from about 15 to about 100 nucleotides, or any integer number therebetween.

In some embodiments, the total length of the first residual circularizing element and the second residual circularizing element is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200 nucleotides.

In some embodiments, the first residual circularizing element and the second residual circularizing element are configured such that the circular RNA comprising them has reduced immunogenicity.

In some embodiments, the first residual circularizing element and the second residual circularizing element are configured such that the circular RNA or circular RNA precursor comprising them has comparable or increased circularization efficiency.

In some embodiments, the first residual circularizing element comprises or consists of from 5′ to 3′ direction a 3′ exon region and optionally a first spacer. In the circular RNA precursor, the 5′ end of the 3′ exon region is directly connected to the 3′ end of the 3′ self-splicing intron fragment.

As used herein, “exon region” in the residual circularizing element is a sequence derived from the native exon of the self-splicing intron (the exon flanking the self-splicing intron) and capable of being recognized and/or spliced by the self-splicing intron (or a combination of the first self-splicing intron fragment and the second self-splicing intron fragment), and thus is required for circularization. An “exon region” can also be referred as a “splicing site sequence” herein.

In some embodiments, the 3′ exon region is derived from the native 3′ exon of the self-splicing intron (the exon flanking (downstream of) the 3′ end of the self-splicing intron) or a contiguous fragment thereof starting from the 5′ terminal nucleotide.

In some embodiments, the 3′ exon region is the entire native 3′ exon of the self-splicing intron, or has at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with the entire native 3′ exon of the self-splicing intron, or has 1, 2, 3, 4, 5, 6, 7, 8, 9, or 10 or more nucleotide substitutions, deletions or additions compared to the entire native 3′ exon of the self-splicing intron.

In some embodiments, the 3′ exon region is a contiguous fragment starting from the 5′ terminal nucleotide of the native 3′ exon. In some embodiments, the 3′ exon region has at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with a contiguous fragment starting from the 5′ terminal nucleotide of the native 3′ exon. In some embodiments, the 3′ exon region has 1, 2, 3, 4, 5, 6, 7, 8, 9, or 10 or more nucleotide substitutions, deletions or additions compared to a continuous fragment starting from the 5′ terminal nucleotide of the native 3′ exon.

In some embodiments, the contiguous fragment starting from the 5′ terminal nucleotide of the native 3′ exon comprises or consists of at least 1%, at least 5%, at least 10%, at least 20%, at least 30%, at least 40%, at least 50%, at least 60%, at least 70%, at least 80%, at least 90% nucleotides of the native 3′ exon. In some embodiments, the contiguous fragment starting from the 5′ terminal nucleotide of the native 3′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 5′ terminal nucleotide of the native 3′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 3′ exon.

In some embodiments, where the self-splicing intron is a Group I intron, the 3′ exon region at least comprises a sequence (such as a sequence of about 1 to about 20 nucleotides) at it 5′ terminus which can pair with the P1 region of the corresponding Group I intron to form a P10 duplex region.

It is believed that for self-splicing of Group I introns, the consecutive one or more nucleotides (such as at least about 1 to about 7 nucleotides) from the 5′ end of the native 3′ exon can pair with the P1 region to form a P10 duplex region, and thus plays an important role in self-splicing. Definitions of the P1 and P10 regions of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the second residual circularizing element comprises or consists of from 3′ to 5′ direction a 5′ exon region and optionally a second spacer. In the circular RNA precursor, the 3′ end of the 5′ exon region is directly connected to the 5′ end of the second self-splicing intron fragment.

In some embodiments, the 5′ exon region is derived from the native 5′ exon of the self-splicing intron (the exon flanking (downstream of) the 5′ end of the self-splicing intron) or a contiguous fragment thereof starting from the 3′ terminal nucleotide.

In some embodiments, the 5′ exon region is the entire native 5′ exon of the self-splicing intron, or has at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with the entire native 5′ exon of the Group I intron, or has 1, 2, 3, 4, 5, 6, 7, 8, 9, or 10 or more nucleotide substitutions, deletions or additions compared to the entire native 5′ exon of the self-splicing intron.

In some embodiments, the 5′ exon region is a contiguous fragment starting from the 3′ terminal nucleotide of the native 5′ exon. In some embodiments, the 5′ exon region has at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with a contiguous fragment starting from the 3′ terminal nucleotide of the native 5′ exon. In some embodiments, the 5′ exon region has 1, 2, 3, 4, 5, 6, 7, 8, 9, or 10 or more nucleotide substitutions, deletions or additions compared to a continuous fragment starting from the 3′ terminal nucleotide of the native 5′ exon.

In some embodiments, the contiguous fragment starting from the 3′ terminal nucleotide of the native 5′ exon comprises or consists of at least 1%, at least 5%, at least 10%, at least 20%, at least 30%, at least 40%, at least 50%, at least 60%, at least 70%, at least 80%, at least 90% nucleotides of the native 5′ exon. In some embodiments, the contiguous fragment starting from the 3′ terminal nucleotide of the native 5′ exon is at least 1 nucleotide in length, such as at least 2, at least 3, at least 4, at least 5, at least 6, at least 7, at least 8, at least 9, at least 10, at least 15 nucleotides, at least 20, at least 25, at least 50 or more nucleotides in length. In some embodiments, the contiguous fragment starting from the 3′ terminal nucleotide of the native 5′ exon is 1 nucleotide in length or up to 2, up to 3, up to 4, up to 5, up to 6, up to 7, up to 8, up to 9, up to 10, up to 15, up to 20, up to 25, up to 50 nucleotides in length or up to the total length of the native 5′ exon.

In some embodiments, where the self-splicing intron is a Group I intron, the 5′ exon region comprises a sequence (for example, a sequence of about 3 to about 8 consecutive nucleotides) at its 3′ terminus which can pair with the internal guide sequence (IGS) of the corresponding Group I intron to form a P1 double-stranded region.

It is believed that for self-splicing of Group I introns, about 3 to about 8 consecutive nucleotides from the 3′ end of the native 5′ exon can pair with the internal guide sequence (IGS) of the intron to form the P1 double-stranded region, thus playing an important role in self-splicing. Definitions of IGS and/or P1 region of Group I introns are known in the art and can be determined, for example, with reference to the following documents: Burke, J. M., et al., (1987) Structural conventions for group I introns; Stahley, R. M., et al (2006) RNA splicing: group I intron crystal structures reveal the basis of splice site selection and metal ion catalysis; and/or Woodson, A. S., (2005) Structure and assembly of group I introns.

In some embodiments, the first residual circularizing element and the second residual circularizing element comprise spacers of different sequences, or one of them comprises a spacer and the other does not.

In some embodiments, the first residual circularizing element and the second residual circularizing element are configured to be capable of forming a stem-loop structure. In some embodiments, the loop of the stem-loop structure comprises the splicing junction.

In some embodiments, the presence of the stem-loop structure can be predicted and/or determined by the nucleotide sequences of the 3′ self-splicing intron fragment (e.g., 3′ Group I intron fragment), the first residual circularizing element, the second residual circularizing element, and the 5′ self-splicing intron fragment (e.g., 5′ Group I intron fragment) involved in circularization. In some embodiments, the presence of a stem-loop structure can be predicted and/or determined from the nucleotide sequence by RNA structure prediction tools such as RNAfold (http://rna.tbi.univie.ac.at/cgi-bin/RNAWebSuite/RNAfold.cgi) or RNAstructure (https://rna.urmc.rochester.edu/RNAstructureWeb/index.html).

In some embodiments, the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure, e.g., through self-splicing for circularization.

Typically, the sequences forming the loop of the stem-loop structure are derived from the 3′ exon region and/or the 5′ exon region.

In some embodiments, where the self-splicing intron is a Group I intron, the first loop sequence comprises or consists of one or more nucleotides (for example, about 1 to about 20 nucleotides) which can pair with the P1 region of the corresponding Group I intron (or the structure formed by the 3′ self-splicing intron fragment and the 5′ self-splicing intron fragment) to form a P10 duplex region during the circularization.

In some embodiments, the first loop sequence may comprise or consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20. In some specific embodiments, n is 2, 4, or 5.

In some embodiments, the first loop sequence comprises or consists of the about 1 to about 7 consecutive nucleotides starting from the 5′ terminal nucleotide of the native 3′ exon of the Group I intron.

In some embodiments, the first loop sequence for example comprises or consists of AAAA, AA, UUUU, UAAA, CAAA, or GAAA.

In some embodiments, where the self-splicing intron is a Group I intron, the second loop sequence comprises or consists of one or more nucleotides (about 3 to about 8 nucleotides) which can pair with the internal guide sequence (IGS) of the corresponding Group I intron (or the structure formed by the 3′ self-splicing intron fragment and the 5′ self-splicing intron fragment) to form a P1 duplex region during the circularization.

In some embodiments, the second loop sequence comprises or consists of the about 3 to about 8 consecutive nucleotides starting from the 3′ terminal nucleotide of the native 5′ exon of the Group I intron.

In some embodiments, the second loop sequence for example comprises or consists of CUU or CUC.

In some embodiments, a loop with a sequence of CUUAAAA, CUUUUUU, CUUAA, CUUGAAA, CUUUAAA, CUUCAAA or CUCAAAA can be formed after circularization.

In some embodiments, the first loop sequence comprises or consists of AAAA and the second loop sequence comprises or consists of CUU. In some specific embodiments, a loop with a sequence of CUUAAAA is formed after circularization.

The pairing sequences forming the stem of the stem-loop structure may be derived from the exon regions, however, it may also be derived from the spacer sequences. Alternatively, the pairing sequence may be derived from an exon region and a spacer sequence, i.e., the pairing sequence comprises at least a portion of an exon region and at least a portion of the spacer.

Without being bound by any theory, the RNA circularization efficiency based on intron self-splicing (e.g., Group I intron self-splicing) is related to the number of base pairs or the type or composition of base pairs in the stem portion of the stem-loop structure formed by the residual circularizing element. The stability of the stem-loop structure (e.g., as can be predicted from calculated free energies) may affect the circularization efficiency.

In some embodiments, the stem portion of the stem-loop structure comprises at least 2 base pairs, such as at least 3, at least 4, at least 5, at least 6, at least 7, at least 8, at least 9, at least 10, at least 11, at least 12, at least 13, at least 14, at least 15 or more base pairs, preferably consecutive matched base pairs. In some embodiments, the stem portion of the stem-loop structure comprises 2-15 or more consecutive matched base pairs. In some embodiments, the stem portion of the stem-loop structure comprises 2, 3, 4, 5, 6, 7, 8, 9, 10, 11, 12, 13, 14, or 15 base pairs, preferably consecutive matched base pairs. In some embodiments, the stem portion of the stem-loop structure comprises 5 base pairs, preferably consecutive matched base pairs. In some embodiments, the stem portion of the stem-loop structure comprises 6 base pairs, preferably consecutive matched base pairs. In some embodiments, the stem portion of the stem-loop structure comprises 7 base pairs, preferably consecutive matched base pairs.

In some embodiments, the stem portion in the stem-loop structure comprises up to 2 base mismatches, or up to 1 base mismatch, preferably, the stem portion comprises no base mismatches.

In some embodiments, the first pairing sequence comprises only G and the second pairing sequence comprises only Cs. In some embodiments, the first pairing sequence comprises only C and the second pairing sequence comprises only G. In some embodiments, the first pairing sequence includes only A and the second pairing sequence includes only U.

In some embodiments, the circular RNA precursor further comprises a 5′ homology arm sequence and a 3′ homology arm sequence capable of complementary pairing to form a homology arm double-stranded region. In some embodiments, the 5′ homology arm sequence is upstream of the 5′ end of the 3′ self-splicing intron fragment and the 3′ homology arm sequence is downstream of the 3′ end of the 5′ self-splicing intron fragment.

In some embodiments, the nucleotide sequence of interest provided herein comprises at least one protein-coding sequence and a translation initiation element such as an internal ribosome entry site (IRES) operably linked thereto. Here, “operably linked” refers to that the translation initiation element such as IRES can mediate translation of the encoded protein. In some embodiments, in the circular RNA precursor, the translation initiation element, such as an IRES, is located upstream of the 5′ end of the at least one protein-coding sequence, or the translation initiation element, such as an IRES, is located downstream of the 3′ end of the at least one protein-coding sequence.

Protein-coding sequences can encode proteins of eukaryotic, prokaryotic or viral origin. In certain embodiments, the protein can be any protein for therapeutic or diagnostic use. For example, the protein coding region can encode human proteins, antigens, antibodies, gene editing enzymes such as CRISPR nucleases, and the like. For example, the encoded protein can be a chimeric antigen receptor, an immunomodulatory protein, and/or a transcription factor, and the like. Some specific examples include, but are not limited to, EGF, FGF1, RBD, G6PC, PAH, HGF, and the like.

In some embodiments, the at least one protein-coding sequence can encode IL-2 or an aquaporin, such as AQP-1 protein or a protein having the same activity as the naturally occurring AQP-1 protein and derived from the naturally occurring AQP-1 protein through substitution, deletion or addition of 1, 2, 3, 4, 5, 6, 7, 8, 9, 10 or more amino acids in the amino acid sequence of the naturally occurring AQP-1 protein. In embodiments, the protein-coding sequence of the present invention encodes the AQP-1 protein. In embodiments, the AQP-1 protein is a mammalian AQP-1 protein, in particular, a human AQP-1 protein.

In some embodiments, the at least one protein-coding sequence of the invention comprises a nucleotide sequence of any one of SEQ ID NO: 117-118, or a nucleotide sequence having at least 50%, at least 60%, at least 70%, at least 80%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.

In some embodiments, the translation initiation element of the invention comprises: IRES sequence, 5′ UTR sequence, Kozak sequence, a sequence containing m6A, a sequence complementary to ribosome 18S rRNA, or any combination thereof.

The IRES sequence may be selected from, but is not limited to, the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2). Wild-type IRES sequences can also be modified and used in the present invention. Preferably, the IRES is CVB3, HRV-B3, BRAV-1_L, PV1_L, CAV2_L, BRAV-1, PV1, or CAV2.

For example, the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 120-526.

In some embodiments, the circular RNA precursor further comprises a homology arm sequence I and a homology arm sequence II capable of complementary pairing to each other to form a homology arm double-stranded region.

In some embodiments, the homology arm sequence I is upstream of the 3′ self-splicing intron fragment and the homology arm sequence II is downstream of the 5′ self-splicing intron fragment.

In some embodiments, the homology arm sequence I or the homology arm sequence II is about 5-500, about 5-450, about 5-400, about 5-350, about 5-300, about 5-250, about 5-200, about 5-150, about 5-100, about 5-80, about 5-60, about 5-50, about 5-40, about 5-30, about 5-25, about 5-20, about 5-15 or about 5-10 nucleotides in length, preferably, about 20, 25, 40, 50, 100, or 300 nucleotides in length.

In some embodiments, the two homology arm sequences may be polyA and polyT, respectively, or polyG and polyC, respectively.

In some embodiments, one of the homology arm sequence I and the homology arm sequence II of the invention comprises a nucleotide sequence of any one of SEQ ID NO: 71-94 or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94, while the other homology arm sequence comprises the corresponding complementary sequence.

In some embodiments, the circular RNA precursor of the invention comprises a nucleotide sequence selected from any one of SEQ ID NO: 527, 532 and 537-542, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 527, 532 and 537-542.

In some embodiments, the nucleotide sequence of interest is a non-coding sequence. For example, the non-coding sequence can be POLR2A, antisense RNA, aptamer, guide RNA, or non-protein-coding RNA existing in any organism, and the like. The non-coding sequence may or may not contain a specific secondary structure.

In one aspect, provided herein is a nucleic acid vector for generating a circular RNA molecule, said vector comprises a coding sequence of the circular RNA precursor of the present invention.

As used herein, “vector” refers to a DNA derived from a virus, plasmid or cell of a higher organism into which a foreign DNA fragment can be or has been inserted for cloning and/or expression purposes. In certain embodiments, the vector can be stably maintained in the organism. The vector may contain, for example, an origin of replication, a selectable marker or a reporter gene, such as antibiotic resistance or GFP, and/or a multiple cloning site (MCS). The term includes linear DNA fragments (e.g., PCR products, linear plasmid fragments), plasmid vectors, viral vectors, cosmids, bacterial artificial chromosomes (BACs), yeast artificial chromosomes (YACs), and the like.

In some embodiments, the nucleic acid vector further comprises an RNA polymerase promoter sequence operably linked to the coding sequence of the circular RNA precursor. The operably linked promoter allows in vivo and/or in vitro transcription of the circular RNA precursor. The promoter is, for example, a T7 RNA polymerase promoter, a T6 viral RNA polymerase promoter, a SP6 viral RNA polymerase promoter, a T3 viral RNA polymerase promoter or a T4 viral RNA polymerase promoter.

There is provided herein a novel Clean PIE system for preparation of circular RNA, which can improve the sequence accuracy of the circular RNA, reduce the change of the secondary structure of circular RNA, thereby decreasing the immunogenicity of the circular RNA, increasing the stability of the circular RNA in cells and reducing the safety risk in clinical applications.

In one aspect, the present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a circularizing sequence fragment II comprising a ribozyme recognition site II at its 5′ end and a circularizing sequence fragment I comprising a ribozyme recognition site I at its 3′ end,
wherein the circularizing sequence fragment I is derived from a 5′ terminal portion of a circularizing sequence, and the circularizing sequence fragment II is derived from a 3′ terminal portion of a circularizing sequence, and
wherein in the generated circular RNA the ribozyme recognition site I is connected with the ribozyme recognition site II, and thus the circularizing sequence fragment I is connected with the circularizing sequence fragment II to form the circularizing sequence.

In one aspect, the present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a circularizing sequence fragment II comprising a ribozyme recognition site II at its 5′ end and a circularizing sequence fragment I comprising a ribozyme recognition site I at its 3′ end,
wherein the circularizing sequence fragment I is the 5′ terminal portion of a circularizing sequence, and the circularizing sequence fragment II is the 3′ terminal portion of a circularizing sequence, and
wherein in the generated circular RNA the ribozyme recognition site I is connected with the ribozyme recognition site II, and thus the circularizing sequence fragment I is connected with the circularizing sequence fragment II to form the circularizing sequence.

In embodiments, the nucleotide sequence of interest comprises one or more sequences selected from the following group between the circularizing sequence fragment II and the circularizing sequence fragment I. at least one protein coding sequences, translation initiation element (TIE), a non-TIE functional element or any combination thereof.

As used herein, in the Clean-PIE system, “circularizing sequence” refers to a nucleotide sequence which is part of the nucleotide sequence of interest. The circularization sequence is involved in or required for circularization by the self-splicing intron and participates in circularization together with the self-splicing intron. In embodiments, the circularizing sequence is a protein-coding sequence, a non-coding sequence, a translation initiation element (TIE), or a non-TIE functional element. As used herein, in the Clean-PIE system, “circularizing sequence fragment I” and “circularizing sequence fragment II” each is part of the circularizing sequence. In the Clean-PIE system of the present invention, circularizing sequence fragments I and II are connected to form the circularizing sequence and such a circularizing sequence formed will retain its biological functions or activities.

As used herein, in the Clean-PIE system, “ribozyme recognition site” refers to a segment of nucleotide sequence which belongs to a part of the circularizing sequence. The ribozyme recognition site is involved in or required for circularization by the self-splicing intron and participates in circularization together with the self-splicing intron but is retained as a part of the circularizing sequence in the final circular RNA. In embodiments, the ribozyme recognition site I is connected with ribozyme recognition II to form the ribozyme recognition site. In embodiments, the ribozyme recognition site comprises 2 to 50 nucleotides, for example 2 to 40, 2 to 30, 2 to 25, 2 to 20, 2 to 15, 2 to 10 nucleotides, in particular 2, 3, 4, 5, 6, 7, 8, 9, 10, 11, 12, 13, 14, 15, 16, 17, 18, 19, 20, 21, 22, 23, 24, 25 or more nucleotides. In embodiments, the ribozyme recognition site I is comprised of 1 to 10 nucleotides, in particular 1, 2, 3, 4, 5, 6, 7, 8, 9 or 10 nucleotides. In embodiments, the ribozyme recognition site II is comprised of 1 to 10 nucleotides, in particular 1, 2, 3, 4, 5, 6, 7, 8, 9 or 10 nucleotides.

As used herein, “a nucleotide motif” refers to a nucleotide sequence pattern that is related to biological function of the ribozyme recognition site. In the present invention, the nucleotide motif is located at the 3′ end of ribozyme recognition site I.

In embodiments, the ribozyme recognition site I comprises or consists of from 5′ to 3′ direction a nucleotide motif consisting of any of SEQ ID NO: 95-116, in particular said nucleotide motif consists of any one of CUU, CUC or CAU.

Only for the purpose of illustrating the present invention, it is provided herein the following examples of the “Clean-PIE system” of the invention. These particular examples include “Clean-PIE-Cargo system”, “Clean-PIE-TIE system”, “Clean-PIE-NonTIE system” and “Clean-PIE-Non-coding Cargo system”. A person skilled in the art understands that any variations, deviations, modifications etc. that are made to the “Clean-PIE system” illustrated as follows do not deviate from the present invention and are thus comprised in the scope of the present invention.

For Clean-PIE-Cargo system, the present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is derived from a 5′ terminal portion of at least one protein-coding sequence, and the protein-coding sequence fragment II is derived from a 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence.

The present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is the 5′ terminal portion of at least one protein-coding sequence, and the protein-coding sequence fragment II is the 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence.

In embodiments, the at least one protein-coding sequence is a sequence encoding a diagnostic or therapeutic protein. In embodiments, the protein encoded by the protein-coding sequence is a mammalian protein, for example a human protein. In embodiments, the at least one protein-coding sequence comprises at least two protein-coding sequences. In embodiments, the at least one protein-coding sequence is a sequence encoding two or more proteins. In embodiments, the at least one protein-coding sequence a sequence encoding two or more diagnostic or therapeutic protein. In some embodiments, the at least one protein-coding sequence comprises at least two protein-coding sequences, and another translation initiation element may be linked between any two adjacent protein-coding sequences, and such another translation initiation element comprises: IRES sequence, 5′ UTR sequence, Kozak sequence, a sequence containing m6A, a sequence complementary to ribosome 18S rRNA, or any combination thereof.

In some embodiments, the at least one protein-coding sequence comprises a nucleotide sequence of any one of SEQ ID NO: 117-118, or a nucleotide sequence having at least 50%, at least 60%, at least 70%, at least 80%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.

It is understood that, in the Clean-PIE-Cargo system of the invention, ribozyme recognition site I and ribozyme recognition site II are respectively a segment of nucleotide sequence which belongs to a part of a cargo sequence, and configured such that the circular RNA comprising them has reduced immunogenicity. In some embodiments, the ribozyme recognition site I and the ribozyme recognition site II are configured such that the circular RNA precursor comprising them has comparable or increased circularization efficiency.

In some embodiments, the total length of the ribozyme recognition site I and the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site I is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the ribozyme recognition site I comprises the sequence structure of the following formula: 5′-third non-pairing sequence-third pairing sequence-third loop sequence-3′; and the ribozyme recognition site II comprises the sequence structure of the following formula: 5′-fourth loop sequence-fourth pairing sequence-fourth non-pairing sequence-3′.

In some examples, the third non-pairing sequence or the fourth non-pairing sequence may be independently present or absent.

In some embodiments, the ribozyme recognition site I comprises or consists of a 'nucleotide motif as defined above. In embodiments, the ribozyme recognition site I comprises or consists of from 5′ to 3′ direction a nucleotide motif, wherein the nucleotide motif consists of any of SEQ ID NO: 95-116, in particular said nucleotide motif consists of any one of CUU, CUC or CAU.

In some embodiments, the ribozyme recognition site I comprises or consists of a nucleotide motif having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.

In some embodiments, the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of GAGGUCUU, AGAGACUU, UCCAGCUU or UCUGGCAU.

In some embodiments, the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AAACCUC, AAUCUCU, AACUGGA or AACCAGC.

In some embodiments, the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.

In embodiments, the ribozyme recognition site I is selected from a nucleotide sequence of UGUAACUU, GAGGUCUU, AGAGACUU, UCCAGCUU, or UCUGGCAU.

In embodiments, the ribozyme recognition site II is selected from a nucleotide sequence of AGAAGUAAC, AAAAUUACA, AAACCUC, AAUCUCU, AACUGGA, orAACCAGC

In embodiments, the ribozyme recognition site I is UGUAACUU, and the ribozyme recognition site II is AGAAGUAAC.

In embodiments, the ribozyme recognition site I is UGUAACUU, and the ribozyme recognition site II is AAAAUUACA.

In embodiments, the ribozyme recognition site I is GAGGUCUU, and the ribozyme recognition site II is AAACCUC.

In embodiments, the ribozyme recognition site I is AGAGACUU, and the ribozyme recognition site II is AAUCUCU.

In embodiments, the ribozyme recognition site I is UCCAGCUU, and the ribozyme recognition site II is AACUGGA.

In embodiments, the ribozyme recognition site I is UCUGGCAU, and the ribozyme recognition site II is AACCAGC.

For Clean-PIE-TIE system, the present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a translation initiation element fragment II, at least one protein-coding sequence, and a translation initiation element fragment I;
wherein the translation initiation element fragment I is derived from a 5′ terminal portion of a translation initiation element, and the translation initiation element fragment II is derived from a 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation.

The present invention provides a circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a translation initiation element fragment II, at least one protein-coding sequence, and a translation initiation element fragment I;
wherein the translation initiation element fragment I is the 5′ terminal portion of a translation initiation element, and the translation initiation element fragment II is the 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation.

In embodiments, the at least one protein-coding sequence is a sequence encoding a diagnostic or therapeutic protein. In embodiments, the protein encoded by the protein-coding sequence is a mammalian protein, for example a human protein. In embodiments, the at least one protein-coding sequence comprises at least two protein-coding sequences. In embodiments, the at least one protein-coding sequence is a sequence encoding two or more proteins. In embodiments, the at least one protein-coding sequence a sequence encoding two or more diagnostic or therapeutic protein. In some embodiments, the at least one protein-coding sequence comprises at least two protein-coding sequences, and another translation initiation element may be linked between any two adjacent protein-coding sequences, and such another translation initiation element comprises: IRES sequence, 5′ UTR sequence, Kozak sequence, a sequence containing m6A, a sequence complementary to ribosome 18S rRNA, or any combination thereof.

In some embodiments, the at least one protein-coding sequence of the invention comprises a nucleotide sequence of any one of SEQ ID NO: 117-118, or a nucleotide sequence having at least 50%, at least 60%, at least 70%, at least 80%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.

It will be understood that, in the Clean-PIE-TIE system of the invention, ribozyme recognition site I and ribozyme recognition site II are respectively a nucleotide sequence that belongs to a part of an IRES sequence, and configured such that the circular RNA comprising them has reduced immunogenicity. In some embodiments, the ribozyme recognition site I and the ribozyme recognition site II are configured such that the circular RNA precursor comprising them has comparable or increased circularization efficiency.

In some embodiments, the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site I is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the ribozyme recognition site I comprises the sequence structure of the following formula: 5′-third non-pairing sequence-third pairing sequence-third loop sequence-3′; and the ribozyme recognition site II comprises the sequence structure of the following formula: 5′-fourth loop sequence-fourth pairing sequence-fourth non-pairing sequence-3′.

In some embodiments, the third non-pairing sequence or the fourth non-pairing sequence may be independently present or absent.

In some embodiments, the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.

In some embodiments, the ribozyme recognition site I comprises or consists of from 5′ to 3′ direction a nucleotide motif consists of any of SEQ ID NO: 95-116, in particular said nucleotide motif consists of any one of CUU, CUC or CAU.

In some embodiments, the ribozyme recognition site II can be selected from various sequence.

In some embodiments, the ribozyme recognition site II comprises or consists of a 3′ exon region as defined above, and the ribozyme recognition site I comprises or consists of a 5′ exon region as defined above.

In some embodiments, the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of GAGGUCUU, AGAGACUU, UCCAGCUU or UCUGGCAU.

In some embodiments, the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AAACCUC, AAUCUCU, AACUGGA or AACCAGC.

In some embodiments, the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.

In embodiments, the ribozyme recognition site I is selected from a nucleotide sequence of UGUAACUU, UGUAACUU, GAGGUCUU, AGAGACUU, UCCAGCUU, or UCUGGCAU.

In embodiments, the ribozyme recognition site II is selected from a nucleotide sequence of AGAAGUAAC, AAAAUUACA, AAACCUC, AAUCUCU, AACUGGA, or AACCAGC.

In embodiments, the ribozyme recognition site I is UGUAACUU, and the ribozyme recognition site II is AGAAGUAAC.

In embodiments, the ribozyme recognition site I is UGUAACUU, and the ribozyme recognition site II is AAAAUUACA.

In embodiments, the ribozyme recognition site I is GAGGUCUU, and the ribozyme recognition site II is AAACCUC.

In embodiments, the ribozyme recognition site I is AGAGACUU, and the ribozyme recognition site II is AAUCUCU.

In embodiments, the ribozyme recognition site I is UCCAGCUU, and the ribozyme recognition site II is AACUGGA.

In embodiments, the ribozyme recognition site I is UCUGGCAU, and the ribozyme recognition site II is AACCAGC.

In one aspect, it is an object of the present invention to provide a circular RNA precursor comprising from 5′ to 3′ direction:
a) a 3′ self-splicing intron fragment;
b) a nucleotide sequence of interest;
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-TIE functional element fragment II, a translation initiation element, and at least one protein-coding sequence, and a non-TIE functional element fragment I;
wherein the non-TIE functional element fragment I is derived from a 5′ terminal portion of a non-TIE functional element, and the non-TIE translation initiation element fragment II is derived from a 3′ terminal portion of the non-TIE functional element, and
the non-TIE functional element fragment I comprises a ribozyme recognition site I located at its 3′ end, the non-TIE functional fragment II comprises a ribozyme recognition site II located at its 5′ end, wherein in the generated circular RNA the ribozyme recognition site I is connected with the ribozyme recognition site II, and thus the non-TIE functional element fragment I is connected with the non-TIE functional element fragment II to form the functional element.

As used herein, the term “non-TIE functional element” refers to a functional element that is not involved in translation initiation, such as a nucleotide sequence for regulating RNA translation, RNA circularization or RNA transcription.

In embodiments, the non-TIE functional element is a 3′ UTR or a replicon. In embodiments, the at least one protein-coding sequence comprises at least two protein-coding sequences. Such at least two protein-coding sequences may connect with each other by spacers or further translation initiation elements. In embodiments, the translation initiation element is selected from an IRES sequence, 5′ UTR sequence, Kozak sequence, a sequence containing m6A modification, a sequence complementary to ribosome 18S rRNA, or any combination thereof. In embodiments, the translation initiation element is an IRES sequence.

In some embodiments, the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site I is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.

In some embodiments, the ribozyme recognition site II can be selected from various sequences.

In some embodiments, the ribozyme recognition site I comprises or consists of a nucleotide motif as defined above. In embodiments, the ribozyme recognition site I comprises or consists of from 5′ to 3′ direction a nucleotide motif, wherein the nucleotide motif consists of any of SEQ ID NO: 95-116, in particular said nucleotide motif consists of any one of CUU, CUC or CAU.

In some embodiments, the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of GAGGUCUU, AGAGACUU, UCCAGCUU or UCUGGCAU.

In some embodiments, the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AAACCUC, AAUCUCU, AACUGGA or AACCAGC.

In some embodiments, the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.

In embodiments, the ribozyme recognition site I is selected from a nucleotide sequence of UGUAACUU, UGUAACUU, GAGGUCUU, AGAGACUU, UCCAGCUU, or UCUGGCAU.

In embodiments, the ribozyme recognition site II is selected from a nucleotide sequence of AGAAGUAAC, AAAAUUACA, AAACCUC, AAUCUCU, AACUGGA, or AACCAGC.

In embodiments, the ribozyme recognition site I is UGUAACUU, and the ribozyme recognition site II is AGAAGUAAC.

In embodiments, the ribozyme recognition site I is UGUAACUU, and the ribozyme recognition site II is AAAAUUACA.

In embodiments, the ribozyme recognition site I is GAGGUCUU, and the ribozyme recognition site II is AAACCUC.

In embodiments, the ribozyme recognition site I is AGAGACUU, and the ribozyme recognition site II is AAUCUCU.

In embodiments, the ribozyme recognition site I is UCCAGCUU, and the ribozyme recognition site II is AACUGGA.

In embodiments, the ribozyme recognition site I is UCUGGCAU, and the ribozyme recognition site II is AACCAGC.

In one aspect, it is an object of the present invention to provide a circular RNA precursor comprising from 5′ to 3′ direction:
a) a 3′ self-splicing intron fragment;
b) a nucleotide sequence of interest;
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-coding sequence fragment II, and a non-coding sequence fragment I;
wherein the non-coding sequence fragment I is derived from a 5′ terminal portion of a non-coding sequence, and the non-coding sequence fragment II is derived from a 3′ terminal portion of the non-coding sequence, and the non-coding sequence fragment I comprises a ribozyme recognition site I located at its 3′ end, the non-coding sequence fragment II comprises a ribozyme recognition site II located at its 5′ end, wherein in the generated circular RNA the ribozyme recognition site I is connected with the ribozyme recognition site II, and thus the non-coding sequence fragment I is connected with the non-coding sequence fragment II to form the complete non-coding sequence.

In embodiments, the non-coding sequence is an endogenous circRNA.

In some embodiments, the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site I is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.

In some embodiments, the ribozyme recognition site II can be selected from various sequences.

In some embodiments, the ribozyme recognition site I comprises or consists of a nucleotide motif as defined above. In embodiments, the ribozyme recognition site I comprises or consists of from 5′ to 3′ direction a nucleotide motif, wherein the nucleotide motif consists of any of SEQ ID NO: 95-116, in particular said nucleotide motif consists of any one of CUU, CUC or CAU.

In some embodiments, the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of GAGGUCUU, AGAGACUU, UCCAGCUU or UCUGGCAU.

In some embodiments, the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AAACCUC, AAUCUCU, AACUGGA or AACCAGC.

In some embodiments, the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.

In embodiments, the ribozyme recognition site I is selected from a nucleotide sequence of UGUAACUU, UGUAACUU, GAGGUCUU, AGAGACUU, UCCAGCUU, or UCUGGCAU.

In embodiments, the ribozyme recognition site II is selected from a nucleotide sequence of AGAAGUAAC, AAAAUUACA, AAACCUC, AAUCUCU, AACUGGA, or AACCAGC.

In embodiments, the ribozyme recognition site I comprises or consists of from 5′ to 3′ direction a nucleotide motif consists of any of SEQ ID NO: 95-116, in particular said nucleotide motif consists of any one of CUU, CUC or CAU.

In embodiments, the ribozyme recognition site I is UGUAACUU, and the ribozyme recognition site II is AGAAGUAAC.

In embodiments, the ribozyme recognition site I is UGUAACUU, and the ribozyme recognition site II is AAAAUUACA.

In embodiments, the ribozyme recognition site I is GAGGUCUU, and the ribozyme recognition site II is AAACCUC.

In embodiments, the ribozyme recognition site I is AGAGACUU, and the ribozyme recognition site II is AAUCUCU.

In embodiments, the ribozyme recognition site I is UCCAGCUU, and the ribozyme recognition site II is AACUGGA.

In embodiments, the ribozyme recognition site I is UCUGGCAU, and the ribozyme recognition site II is AACCAGC.

In some embodiments, the nucleotide sequence of interest is at least 10, 20, 40, 60, 80, 100, 200, 300, 400, 500, 600, 700, 800, 900, 1000, 1500, 2000, 2500, 3000, 3500, 4000, 4500, 10000, 20000 nucleotides in length.

In some embodiments, the circular RNA is at least 10, 20, 40, 60, 80, 100, 200, 300, 400, 500, 600, 700, 800, 900, 1000, 1500, 2000, 2500, 3000, 3500, 4000, 4500, 5000, 10000, 20000 nucleotides in length. In some embodiments, the circular RNA is at least about 10 nucleotides in length. In some embodiments, the circular RNA is about 50 nt or less. In some embodiments, the circular RNA is about 80 nt or less. In some embodiments, the circular RNA is about 100 nt or less. In some embodiments, the circular RNA is about 150 nt or less. In some embodiments, the circular RNA is about 200 nt or less. In some embodiments, the circular RNA is about 250 nt or less. In some embodiments, the circular RNA is about 300 nt or less. In some embodiments, the circular RNA is about 350 nt or less. In some embodiments, the circular RNA is about 400 nt or less. In some embodiments, the circular RNA is about 450 nt or less. In some embodiments, the circular RNA is about 500 nt or less. In some embodiments, the circular RNA is about 600 nt or less. In some embodiments, the circular RNA is about 700 nt or less. In some embodiments, the circular RNA is about 800 nt or less. In some embodiments, the circular RNA is about 900 nt or less. In some embodiments, the circular RNA is at least about 1000 nt.

In further aspect, the present invention provides a pharmaceutical composition comprising the circular RNA precursor of the invention and/or the nucleic acid vector of the invention and/or the circular RNA of the invention, and a pharmaceutically acceptable carrier.

In some embodiments, a pharmaceutically acceptable carrier may include, but are not limited to, buffers, excipients, stabilizers or preservatives. Examples of pharmaceutically acceptable carriers are physiologically compatible solvents, dispersion media, coatings, antibacterial and antifungal agents, isotonic and absorption delaying agents, and the like, such as salts, buffers, carbohydrates, antioxidants, aqueous or non-aqueous carriers, preservatives, wetting agents, surfactants or emulsifying agents or combinations thereof. The amount of a pharmaceutically acceptable carrier in a pharmaceutical composition can be determined experimentally based on the activity of the carrier and the desired properties of the formulation, such as stability and/or minimal oxidation.

In another aspect, the present invention provides a method for preparing a circular RNA, the method comprises:
1) providing a circular RNA precursor of the present invention or obtaining a circular RNA precursor by transcribing from the nucleic acid vector of the present invention;
2) incubating the circular RNA precursor in the presence of a divalent metal cation at a temperature at which RNA circularization occurs; and
3) harvesting the circular RNA obtained in step 2).

In some embodiments, the divalent metal cation is Mg²⁺ and/or Mn²⁺. In some embodiments, the concentration of the divalent metal cation is at least about 5 mM, e.g., about 5 mM to about 550 mM, e.g., at least about 5 mM, about 10 mM, about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In another aspect, the present invention provides a method for preparing a circular RNA, the method comprises
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.

In some embodiments, the nucleic acid vector is the nucleic acid vector of the present invention.

In some embodiments, the divalent metal cation in the in vitro transcription system is Mg²⁺. In some embodiments, the in vitro transcription system further comprises a monovalent metal cation and/or a monovalent anion.

In some embodiments, the monovalent metal cation is Na⁺ or K⁺.

In some embodiments, the monovalent metal anion is Cl⁻ or CH₃COO⁻ (OAc).

In some embodiments, the concentration of the divalent metal cation in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM. In some embodiments, the concentration of the divalent metal cation in the system during the first time period is 30 mM.

In some embodiments, the concentration of the monovalent metal cation in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM. In some embodiments, the monovalent metal cation is Na⁺, the concentration in the system during the first time period is 15 mM. In some embodiments, the monovalent metal cation is K⁺, the concentration in the system during the first time period is 90 mM.

In some embodiments, the concentration of the monovalent anion in the system during the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM, about 100 mM, about 150 mM. In some embodiments, the monovalent anion is Cl⁻, the concentration in the system during the first time period is 90 mM. In some embodiments, the monovalent anion is CH₃COO⁻ (OAc), the concentration in the system during the first time period is 125 mM.

In some embodiments, the in vitro transcription and self-circularization occur in the same reaction system.

In some embodiments, the method does not include a step of isolating and/or purifying the linear RNA produced by the in vitro transcription.

Those skilled in the art would know that the in vitro transcription system also comprises various components required for transcription, such as buffers, rATP, rCTP, rUTP, rGTP and the like.

In some embodiments, the buffer of the in vitro transcription system is Tris-HCl buffer, or HEPES buffer, or MES buffer, or citrate buffer, or phosphate buffer. In some embodiments, the buffer of the in vitro transcription system is HEPES buffer.

In some embodiments, the in vitro transcription system has a pH of about 5-about 8, such as a pH of about 5, about 5.5, about 6, about 6.5, about 7, about 7.5, or about 8. In some embodiments, the in vitro transcription system has a pH of about 7.5.

The RNA polymerase depends on the promoter used in the nucleic acid vector to drive transcription. The RNA polymerase may include, but is not limited to, a T7 RNA polymerase, a T6 viral RNA polymerase, a SP6 viral RNA polymerase, a T3 viral RNA polymerase, or a T4 viral RNA polymerase. In some embodiments, the RNA polymerase is T7 RNA polymerase.

In some embodiments, the first time period is at least 0.5 hours, such as about 0.5 hours to about 24 hours, such as about 0.5 hours, about 1 hour, about 1.5 hours, about 2 hours, about 2.5 hours, about 3 hours, about 3.5 hours, about 4 hours, about 5 hours, about 10 hours, or about 24 hours. In some embodiments, the first time period is 3 hours.

In some embodiments, the incubation for the first time period is carried out at about 16° C. to about 60° C., such as at about 16° C., about 17° C., about 18° C., about 19° C., about 20° C. ° C., about 21° C., about 22° C., about 23° C., about 24° C., about 25° C., about 26° C., about 27° C., about 28° C., about 29° C., about 30° C. ° C., about 31° C., about 32° C., about 33° C., about 34° C., about 35° C., about 36° C., about 37° C., about 38° C., about 39° C., about 40° C. ° C., about 41° C., about 42° C., about 43° C., about 44° C., about 45° C., about 46° C., about 47° C., about 48° C., about 49° C., about 50° C. ° C., about 51° C., about 52° C., about 53° C., about 54° C., about 55° C., about 56° C., about 57° C., about 58° C., about 59° C., or about 60° C. In some embodiments, the incubation for the first time period is carried out at about 37° C.

In some embodiments, after incubation for the first time period in step b), the method further comprises step c):
adding an additional amount of metal cation to the in vitro transcription system and incubating for a second time period; or
changing the buffer of the system, adding a metal cation and incubating for a second time period.

In some embodiments, the metal cation added for the incubation of the second time period is a divalent metal cation, such as Mg²⁺ or Mn²⁺.

In some embodiments, during the incubation of the second time period, the metal cation is added to a final concentration of at least about 5 mM, such as about 5 mM to about 550 mM, such as at least about 5 mM, at least about 10 mM, at least about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In some embodiments, the buffer of the system is changed to Tris-HCl buffer, or HEPES buffer, or MES buffer, or citrate buffer, or phosphate buffer during the second time period.

In some embodiments, the pH of the system in the second time period is 5-8, such as pH 5, pH 5.5, pH 6, pH 6.5, pH 7, pH 7.5, or pH 8.

In some embodiments, the second time period is at least 5 minutes, such as about 5 minutes to about 2 hour, such as about 5 minutes, about 10 minutes, about 15 minutes, about 20 minutes, about 25 minutes, about 30 minutes, about 45 minutes, about 50 minutes, about 55 minutes, about 60 minutes, about 120 minutes or more.

In some embodiments, wherein the reaction for the second time period is carried out at about 25° C. to about 75° C., such as at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 50° C., about 55° C., about 60° C., about 65° C., about 70° C., or about 75° C.

In some embodiments, the method further comprises a step of recovering or purifying the circular RNA as produced.

In one aspect, the present invention provides a circular RNA produced by the method of the invention.

In one aspect, the present invention provides an in vitro transcription method comprising:
a) providing a nucleic acid vector as a template for in vitro transcription;
b) incubating the nucleic acid vector in an in vitro transcription system comprising a divalent metal cation and an RNA polymerase for a first period of time; and
c) i) adding an additional amount of metal cation to the system, or ii) changing the buffer of the system and adding a metal cation to the system, and incubating the system for a second period of time.

In some embodiments, the in vitro transcription system during the first time period further comprises a monovalent metal cation and/or a monovalent anion.

In some embodiments, the divalent metal cation in the in vitro transcription system during the first time period is Mg²⁺.

In some embodiments, the monovalent metal cation during the first time period is Na⁺ or K⁺.

In some embodiments, the monovalent metal anion during the first time period is Cl⁻ or CH₃COO⁻ (OAc).

The RNA polymerase depends on the promoter used in the nucleic acid vector to drive transcription. The RNA polymerase may include, but is not limited to, a T7 RNA polymerase, a T6 viral RNA polymerase, a SP6 viral RNA polymerase, a T3 viral RNA polymerase, or a T4 viral RNA polymerase. In some embodiments, the RNA polymerase is T7 RNA polymerase.

In some embodiments, the concentration of the divalent metal cation in the system for the first time period is from about 5 mM to about 50 mM, e.g., about 5 mM, about 10 mM, about 15 mM, about 20 mM, about 25 mM, about 30 mM, about 35 mM, about 40 mM, about 45 mM, about 50 mM.

In some embodiments, the concentration of the monovalent metal cation in the system for the first time period is from about 5 mM to about 100 mM, e.g., about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM.

In some embodiments, the concentration of the monovalent anion in the system for the first time period is from about 5 mM to about 150 mM, e.g., about 5 mM, about 10 mM, about 15 mM, about 20 mM, about 25 mM, about 30 mM, about 35 mM, about 40 mM, about 45 mM, about 50 mM, about 100 mM, about 150 mM.

In some embodiments, the buffer of the in vitro transcription system for the first time period is Tris-HCl buffer, or HEPES buffer, or MES buffer, or citrate buffer, or phosphate buffer.

In some embodiments, the pH of the in vitro transcription system for the first time period is 5-8, such as pH 5, pH 5.5, pH 6, pH 6.5, pH 7, pH 7.5, or pH 8.

In some embodiments, the first time period is at least 0.5 hours, such as about 0.5 hours to about 24 hours, such as about 0.5 hours, about 1 hour, about 1.5 hours, about 2 hours, about 2.5 hours, about 3 hours, about 3.5 hours, about 4 hours, about 5 hours, about 10 hours, or about 24 hours.

In some embodiments, the incubation for the first time period is carried out at about 16° C. to about 60° C., such as at about 16° C., about 17° C., about 18° C., about 19° C., about 20° C. ° C., about 21° C., about 22° C., about 23° C., about 24° C., about 25° C., about 26° C., about 27° C., about 28° C., about 29° C., about 30° C. ° C., about 31° C., about 32° C., about 33° C., about 34° C., about 35° C., about 36° C., about 37° C., about 38° C., about 39° C., about 40° C. ° C., about 41° C., about 42° C., about 43° C., about 44° C., about 45° C., about 46° C., about 47° C., about 48° C., about 49° C., about 50° C. ° C., about 51° C., about 52° C., about 53° C., about 54° C., about 55° C., about 56° C., about 57° C., about 58° C., about 59° C., or about 60° C.

In some embodiments, the added metal cation in the system for the second time period is a divalent metal cation, such as Mg²⁺ or Mn²⁺.

In some embodiments, during the second time period, the metal cation is added to a final concentration of at least about 5 mM, such as about 5 mM to about 550 mM, such as at least about 5 mM, at least about 10 mM, at least about 15 mM, at least about 20 mM, at least about 30 mM, at least about 40 mM, at least about 50 mM, at least about 60 mM, at least about 70 mM, at least about 80 mM, at least about 90 mM, at least about 100 mM, at least about 125 mM, at least about 150 mM, at least about 175 mM, at least about 200 mM, at least about 250 mM, at least about 300 mM, at least about 350 mM, at least about 400 mM, at least about 450 mM, at least about 500 mM, at least about 550 mM or higher.

In some embodiments, the second time period is at least 5 minutes, such as about 5 minutes to about 2 hour, such as about 5 minutes, about 10 minutes, about 15 minutes, about 20 minutes, about 25 minutes, about 30 minutes, about 45 minutes, about 50 minutes, about 55 minutes, about 60 minutes, about 120 minutes or more.

In some embodiments, the incubation for the second time period is carried out at about 25° C. to about 75° C., such as at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 50° C., about 55° C., about 60° C., about 65° C., about 70° C., or about 75° C.

In some embodiments, the method further comprises a step of recovering and/or purifying the RNA obtained in step c).

In one aspect, the present invention provides an RNA produced by the method of the invention.

In another aspect, the present invention provides a method for purifying a circular RNA, the method comprises:
a) contacting a mixture comprising circular RNA and the circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.

In some embodiments, the circular RNA is prepared by circularizing a linear circular RNA precursor. In some embodiments, the circular RNA is prepared by ligating both ends of a linear circular RNA precursor with an RNA ligase, such as a T4 RNA ligase. In some embodiments, the circular RNA is prepared by the self-splicing ribozyme activity of Group I intron-based circularizing elements contained in the linear circular RNA precursor, e.g., the circular RNA is the circular RNA described in Section I herein or any clause below and/or prepared by the method described in Section II herein or any clause below.

In some embodiments, the circular RNA-specific probe is a single-stranded DNA probe.

In some embodiments, the circular RNA-specific probe is a single-stranded RNA probe.

In some embodiments, the circular RNA-specific probe specifically hybridizes to a region flanking the circularization junction of the circular RNA.

In some embodiments, the circular RNA-specific probe is at least 10 nucleotides, at least 12 nucleotides, at least 14 nucleotides, at least 16 nucleotides, at least 18 nucleotides in length acid, at least 20 nucleotides, at least 25 nucleotides, at least 30 nucleotides, at least 35 nucleotides in length or longer, for example, the circular RNA-specific probe is 16, 17, 18, 19, 20, 21, 22, 23, 24, 25, 25, 27, 28, 29 or 30 nucleotides in length.

In some embodiments, the circular RNA-specific probe is immobilized on a support such as a solid support, e.g., the circular RNA-specific probe is immobilized on the support after binding to the circular RNA or the circular RNA-specific probe is pre-immobilized on the support.

In some embodiments, the condition in step a) include denaturing the RNA at between about 60° C. and about 95° C. (e.g., about 60° C., about 62° C., about 64° C., about 66° C., about 68° C., about 70° C., about 75° C., about 80° C., about 85° C., about 90° C., about 95° C.) for about 2 minutes to about 10 minutes (e.g., about 2, 3, 4, 5, 6, 7, 8, 9, or 10 minutes), then gradually reducing the temperature to below about 40° C. (e.g., below about 35° C., below about 30° C., below about 25° C., below about 20° C. or less) to allow the circular RNA annealing to the circular RNA-specific probe.

In some embodiments, the condition in step a) include a high salt concentration range of 0.25M-2M, e.g., 0.25M, 0.5M, 0.75M, 1M, 1.25M, 1.5M, 1.75M or 2M. In some embodiments, the salt is NaCl or a guanidine salt (e.g., Guanidine hydrochloride).

In some embodiments, in step b), the one or more components are removed by washing the complex with a washing buffer.

In some embodiments, step c) is performed by increasing the temperature to about 60° C. to about 95° C. (e.g., about 60° C., about 62° C., about 64° C., about 66° C., about 68° C.) C, about 70° C., about 75° C., about 80° C., about 85° C., about 90° C., about 95° C.) to release the circular RNA.

In some embodiments, in step c) the circular RNAis released by elution with an elution buffer.

In some embodiments, the elution buffer is a low salt buffer, e.g., a buffer with salt concentration lower than 0.5M. In some embodiment, the elution buffer is Tris-EDTAbuffer (TE buffer) or water.

In some embodiments, the method further includes the following steps:
i) contacting the mixture comprising circular RNA and the circular RNA precursor to a linear circular RNA precursor-specific probe in a condition that allows the linear circular RNA precursor-specific probe to specifically bind to and form a complex with the linear circular RNA precursor;
ii) removing the complex formed by the linear circular RNA precursor-specific probe with the linear circular RNA precursor from the mixture, and
iii) collecting the circular RNA-containing mixture obtained in step ii).

In some embodiments, steps i)-iii) are performed before step a), for example, steps i)-iii) may be performed multiple times before step a), e.g., 2, 3, 4 or more times. In some embodiments, steps i)-iii) are performed concurrently with steps a)-c).

In one aspect, the present invention provides a method for purifying circular RNA, the method comprises:
i) contacting a mixture comprising circular RNA and the circular RNA precursor to a linear circular RNA precursor-specific probe under a condition that allows the linear circular RNA precursor-specific probe to specifically bind to and form a complex with the linear circular RNA precursor; and
ii) removing the complex formed by the linear circular RNA precursor-specific probe with the linear circular RNA precursor from the mixture,
iii) collecting the circular RNA-containing mixture obtained in step ii), and optionally, steps i)-iii) are performed multiple times, e.g., 2 times, 3 times, 4 times or more.

In some embodiments, the linear circular RNA precursor-specific probe specifically binds to the linear circular RNA precursor and does not substantially bind to the circular RNA.

In some embodiments, the linear circular RNA precursor-specific probe is immobilized on a support, such as a solid support, for example, the linear circular RNA precursor-specific probe is then immobilized on the support after binding to the linear circular RNA precursor, or the linear circular RNA precursor-specific probe is pre-immobilized on the support.

In some embodiments, the circular RNA-specific probe specifically hybridizes to at least a portion of the first residual circularizing element and a portion of the second residual circularizing element.

In some embodiments, the linear precursor RNA-specific probe hybridizes to a portion of the linear circular RNA precursor outside the first residual circularizing element, the nucleotide sequence of interest, and the second residual circularizing element.

In some embodiments, the linear precursor RNA-specific probe hybridizes to the 3′ self-splicing intron fragment or a portion thereof or a 5′ flanking sequence thereof, or the 5′ self-splicing intron fragment or a portion thereof or a 3′ flanking sequence thereof.

In some specific embodiments, the linear circular RNA precursor contains a sequence selected from SEQ ID NOs:600-610 or a complement sequence thereof, preferably, SEQ ID NO:606 or a complement sequence thereof outside the first residual circularizing element, the nucleotide sequence of interest, and the second residual circularizing element, to which the linear precursor RNA-specific probe specifically hybridizes.

In some embodiments of the method for purifying circular RNA of the invention, the molar ratio of the probe to the RNA molecules in the mixture is from about 1:1 to about 100,000:1.

In some embodiments, the linear circular RNA precursor-specific probe hybridizes to a 3′ homology arm sequence or portion thereof on the linear circular RNA precursor, or a 5′ homology arm sequence or portion thereof on the linear circular RNA precursor. In some embodiments, the homology arm sequence comprises polyA, polyU, polyC or polyG. In some embodiments, the homology arm sequence is about 10-about 200 nt in length. Accordingly, in some embodiments, the linear precursor RNA-specific probe comprises polyU, poly A, polyG or polyC. In some embodiments, the linear precursor RNA-specific probe is about 10-about 200 nt in length.

In one aspect, the present invention provides a method for purifying circular RNA, the method comprises:
i) adding a linear RNA-specific tag to a mixture comprising circular RNA and linear RNA, wherein the linear RNA-specific tag can be linked to the linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii), optionally, steps ii)-iv) are performed multiple times, e.g., 2 times, 3 times, 4 times or more.

In some embodiments, the tag comprises a polyA, polyG, polyU, or polyC sequence. Accordingly, the probe includes a polyT/polyU, polyC, polyA, or polyG sequence. Alternatively, the tag can be a random sequence, and the probe comprises a sequence complementarily paired with said random sequence.

In some embodiments, the tag may be about 10-200 nt in length. In some embodiments, the probe may be about 10-200 nt in length.

In some embodiments, the tag is added to the linear RNA by adding a PolyA/T/C/G polymerase or a ligase to the mixture. In some embodiments, adding rATP, rGTP, rUTP, rCTP or rNTP, or a random tag sequence of about 10-200 nt to the mixture is also included.

In some embodiments, the linear RNA probe specifically binds to the added tag without substantially binding to the circular RNA. In some embodiments, the linear RNA probe is a single-stranded DNA probe, or a single-stranded RNA probe.

In some embodiments, the linear RNA probe is immobilized on a support such as a solid support, for example, the linear RNA probe is immobilized on the support after binding to the linear RNA, or, the linear RNA probe is pre-immobilized on the support.

In some embodiments, the present invention provides a method of screening a target open reading frame (ORF) sequence encoding a target polypeptide for a nucleotide motif, the method comprises steps of:
1) extracting amino acid units in the direction of N terminal to C terminal in the target polypeptide containing m amino acids, and each group of amino acid units includes an amino acid with the number of n; with a step of 1 amino acid, slide along the N terminal to the C terminal, n is an integer and n≥2, m is an integer and ≥1;
2) listing all nucleotide sequences corresponding to each group of amino acid units according to their degenerate codon; and
3) identifying nucleotide sequences obtained from step 2) that matches the nucleotide motif;
wherein the nucleotide motif consists of any one of SEQ ID NO: 95-116, preferably CUU, CUC or CAU, and
4) optionally, selecting the target ORF as the circularizing sequence of a circular RNA precursor.

In some embodiments, the present invention provides a system for screening a target open reading frame (ORF) for a nucleotide motif, wherein the target open reading frame (ORF) is a nucleotide sequence encoding a target polypeptide, and the system comprises:
a) an amino acid extraction module, which is used for extracting amino acid units in the direction of N terminal to C terminal in the target polypeptide containing m amino acids, wherein each group of amino acid units comprise an amino acid with a number of n, with a step of 1 amino acid, sliding along the N terminal to the C terminal, and n is an integer and n≥2, m is an integer and ≥1;
b) a conversion module, which is used for determining the set of codon sequences of each amino acid unit, each set of codon sequences includes all degenerate codon sequence corresponding to each set of amino acid units; and
c) a target codon sequence filtering module, which is used for determining whether the target codon sequence contain a nucleotide sequence that exactly matches the nucleotide motif,
wherein the nucleotide motif consists of any one of SEQ ID NO: 95-116, preferably CUU, CUC or CAU.

In some embodiments, the present invention provides a method of screening a target non-coding sequence for a nucleotide motif, comprising a step of:
1) editing 0, 1, or 2 bases on the non-coding sequence so that the non-coding sequence comprises an edited region that matches the nucleotide motif whilst maintaining the non-coding sequence's function;
wherein the nucleotide motif consists of a nucleotide sequence of any one of SEQ ID NO: 95- 116, preferably CUU, CUC or CAU.; and
2) optionally, selecting the target non-coding sequence as the circularizing sequence of a circular RNA precursor, wherein the target non-coding sequence is an endogenous circRNA.

In some embodiments, the present invention provides a system for screening a target non-coding sequence for a nucleotide motif, wherein the target non-coding sequence is an endogenous circRNA, and the system comprises:
a) a base editing module, which is used for editing the bases at any one or more positions in the target non-coding sequence to obtain an edited region matching the nucleotide motif,
wherein the nucleotide motif consists of any one of SEQ ID NO: 95-116, preferably CUU, CUC or CAU.

In some embodiments, the present invention provides a method for screening a target internal ribosome entry site (IRES) for a nucleotide motif, comprising steps of:
1) identifying regions of non-conserved sequences of the target IRES by comparing the target IRES with the other IRES sequences from the same viral subgenus, through multiple sequence alignment tools, for example: Clustal Omega, or MUSCLE;
2) based on the results of multiple sequence alignment, determining a low-structure region of the target IRES through secondary structure prediction software, such as RNAfold, Mfold, RNAstructure or RNAalifold; and
3) editing 0, 1, or 2 bases in the low-structure region in the target IRES to match the nucleotide motif without affecting the translation initiation activity of said IRES;
wherein the nucleotide motif consists of a nucleotide sequence of any one of SEQ ID NO: 95-116, preferably CUU, CUC or CAU.

As used herein, the term “low-structure region” refers to a region that does not have a stem-loop structure, a region having high free energy, or a single strand region.

In some embodiments, the present invention provides a system for screening a target internal ribosome entry site (IRES) for a nucleotide motif, wherein the system comprises:
a) a multi-sequence alignment module, which is used for comparing the target IRES with the other IRES sequences from the same viral subgenus to identify regions of non-conservative sequences;
b) a low-structure prediction module, which is used for determining a low-structure region within the target IRES according to the result of the multiple sequence alignments of the target IRES; and
c) a base editing module, which is used for editing the bases at any one or more positions in the region to obtain an edited region matching the nucleotide motif,
wherein the nucleotide motif consists of a nucleotide sequence of any one of SEQ ID NO: 95-116, preferably CUU, CUC or CAU.

In some embodiments, the present invention provides a screening method for screening a target non-TIE functional element for the nucleotide motif in ribozyme recognition site I, comprising a step of:
a) editing 0, 1, or 2 bases on the non-TIE functional element so that the non-TIE functional element sequence (such as 3′ UTR or a replicon) comprises an edited region that matches the nucleotide motif whilst maintaining the non-TIE function;
wherein the nucleotide motif consists of a nucleotide sequence of any one of SEQ ID NO: 95-116, preferably CUU, CUC or CAU.

In some embodiments, the present invention provides a system for screening a target region sequence in a non-TIE functional element sequence (such as 3′UTR or a replicon) for a nucleotide motif, wherein the screening system comprises:
a) a base editing module, which is used for editing the bases at any one or more positions in the region to obtain an edited region matching the nucleotide motif,
wherein the nucleotide motif consists of a nucleotide sequence of any one of SEQ ID NO: 95-116, preferably CUU, CUC or CAU.

In another aspect, there is provided a method for preventing or treating a disease or condition in a subject, comprising administering to a subject the circular RNA of the invention, or the pharmaceutical composition of the invention.

In some embodiments, the disease or condition comprises but not limited to xerostomia, xerophthalmia, Sjogren's syndrome or the like.

Sjogren's syndrome is a systemic autoimmune disease in which immune cells attack and destroy the exocrine glands that produce saliva and tears. Sjögren's syndrome can also affect multiple organs, including kidneys and lungs. Sjögren's syndrome can occur in all age groups of both women and men. Sjögren's syndrome can occur independently, referred to as primary Sjögren's syndrome, or may develop years after the onset of an associated rheumatic disorder, referred to as secondary Sjögren's syndrome.

Xerostomia (dry mouth) and xerophthalmia (conjunctivitis sicca, dry eyes) are hallmarks of Sjögren's syndrome. Immunologically-activated or apoptotic glandular epithelial cells that expose autoantigens in predisposed individuals might drive autoimmune-mediated tissue injury. Immune activation is typically presented as focal, mononuclear (T, B and macrophage) cell infiltrates proximal to the ductal epithelial cells (epithelitis) and forms sialadenitis. Though the pathogenetic mechanism for this autoimmune exocrinopathy has not been fully elucidated, it has been shown that CD4+ T-lymphocytes constitute 60-70 percent of the mononuclear cells infiltrating the glands. Abnormal activation of proinflammatory Th1 and Th17 cells have been reported to be central to induction of SS in either human or animal models.

The present invention provides a novel therapy to prevent or treat Sjögren's syndrome-related xerostomia in a subject. The inventors have discovered that administration of a circular RNA comprising a nucleotide sequence that encodes an aquaporin-1 (AQP-1) protein or variants thereof to a subject can prevent or treat Sjögren's syndrome-related xerostomia in a subject.

In embodiments, a protein useful in the present invention is an AQP-1 protein or variants thereof, wherein such AQP1 protein or variants thereof refers to a naturally occurring AQP-1 protein, or a protein having the same activity as the naturally occurring AQP-1 protein and derived from the naturally occurring AQP-1 protein through substitution, deletion or addition of 1, 2, 3, 4, 5, 6, 7, 8, 9, 10 or more amino acids in the amino acid sequence of the naturally occurring AQP-1 protein.

In some embodiments, an AQP-1 protein or variants thereof comprises a sequence having at least 50%, at least 55%, at least 60%, at least 65%, at least 70%, at least 75%, at least 80% at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the entire amino acid sequence of a naturally occurring AQP-1 protein. In embodiments, an AQP-1 protein is a portion of the amino acid sequence of an AQP-1 protein, wherein such portion of an AQP-1 protein retains the ability to form a channel in a cell membrane that allows the passage of water. It is also known in the art that several isoforms of AQP-1 protein exist. Thus, in embodiments, an AQP-1 protein is an isoform of an AQP-1 protein, wherein such isoform retains the ability to form a channel that allows the passage of water. In embodiments, an AQP-1 protein is a portion of an isoform or other naturally-occurring variant of an AQP-1 protein, wherein such portion retains the ability to form a channel in a membrane that allows the passage of water. Methods to produce functional portions and variants of AQP-1 proteins, such as conservative variants, of AQP-1 protein are known to those skilled in the art.

In some embodiments, the present invention provides a circular RNA precursor comprising from 5′ to 3′ direction:
a) a 3′ self-splicing intron fragment;
b) a nucleotide sequence of interest, comprising from 5′ to 3′ direction: protein-coding sequence fragment II, a translation initiation element such as an internal ribosome entry site (IRES) and a protein-coding sequence fragment I; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the protein-coding sequence fragment II, the translation initiation element, and the protein-coding sequence fragment I through the self-splicing of the circular RNA precursor;
wherein the protein-coding sequence fragment I is derived from a 5′ terminal portion of at least one protein-coding sequence, and the protein-coding sequence fragment II is derived from a 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence;
wherein the at least one protein-coding sequence encodes an aquaporin, such as AQP-1 protein or variants thereof.

In some embodiments, the present invention provides a circular RNA precursor comprising from 5′ to 3′ direction:
a) a 3′ self-splicing intron fragment;
b) a nucleotide sequence of interest, comprising from 5′ to 3′ direction: protein-coding sequence fragment II, a translation initiation element such as an internal ribosome entry site (IRES) and a protein-coding sequence fragment I; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the protein-coding sequence fragment II, the translation initiation element, and the protein-coding sequence fragment I through the self-splicing of the circular RNA precursor;
wherein the protein-coding sequence fragment I is the 5′ terminal portion of at least one protein-coding sequence, and the protein-coding sequence fragment II is the 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence;
wherein the at least one protein-coding sequence encodes an aquaporin, such as AQP-1 protein or variants thereof.

In embodiments, the present invention provides a circular RNA precursor comprising from 5′ to 3′ direction:
a) a 3′ self-splicing intron fragment;
b) a translation initiation element (TIE) fragment II;
c) a nucleotide sequence that encodes an aquaporin-1 (AQP-1) protein or variants thereof;
d) a translation initiation element fragment I and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the TIE fragment I is derived from a 5′ terminal portion of a TIE sequence, and the TIE fragment II is derived from a 3′ terminal portion of a TIE sequence, and
the TIE fragment I comprises a ribozyme recognition site I located at its 3′ end, the TIE fragment II comprises a ribozyme recognition site II located at its 5′ end, wherein in the generated circular RNA the ribozyme recognition site I is connected with the ribozyme recognition site II, and thus the TIE I is connected with the TIE II to form the TIE sequence which is able to recruit ribosome to initiate translation.

In embodiments, the present invention provides a circular RNA precursor comprising from 5′ to 3′ direction:
a) a 3′ self-splicing intron fragment;
b) a translation initiation element (TIE) fragment II;
c) a nucleotide sequence that encodes an aquaporin-1 (AQP-1) protein or variants thereof;
d) a translation initiation element fragment I and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the TIE fragment I is the 5′ terminal portion of a TIE sequence, and the TIE fragment II is the 3′ terminal portion of a TIE sequence, and
the TIE fragment I comprises a ribozyme recognition site I located at its 3′ end, the TIE fragment II comprises a ribozyme recognition site II located at its 5′ end, wherein in the generated circular RNA the ribozyme recognition site I is connected with the ribozyme recognition site II, and thus the TIE I is connected with the TIE II to form the TIE sequence which is able to recruit ribosome to initiate translation.

In some embodiments, the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At).

In some embodiments, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.

In some embodiments, the self-splicing intron is selected from a nucleotide sequence of any one of SEQ ID NO: 1 and 3, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1 and 3.

In some embodiments, the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.

In some embodiments, the self-splicing intron is the Staphylococcus phage Twort Group I Intron (Twort), the 3′ self-splicing intron fragment comprises or consists of a nucleotide sequence of SEQ ID NO: 27 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 27, and the 5′ self-splicing intron fragment comprises or consists of a nucleotide sequence of SEQ ID NO: 28 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 28.

In some embodiments, the total length of the ribozyme recognition site I and the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site I is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the total length of the ribozyme recognition site II is about 0 to about 100 nucleotides, about 0 to about 90 nucleotides, about 0 to about 80 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

In some embodiments, the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 58, 60 or 64.

In some embodiments, the ribozyme recognition site I comprises a nucleotide sequence of UCCAGCUU, UCUGGCAU or UGUAACUU.

In some embodiments, the ribozyme recognition site II comprises a nucleotide sequence of AACUGGA, AACCAGC or AAAAUUACA.

In embodiments, the ribozyme recognition site I is UCCAGCUU, and the ribozyme recognition site II is AACUGGA.

In embodiments, the ribozyme recognition site I is UCUGGCAU, and the ribozyme recognition site II is AACCAGC.

In embodiments, the ribozyme recognition site I is UGUAACUU, and the ribozyme recognition site II is AAAAUUACA.

In some embodiments, the circular RNA precursor further comprises a homology arm sequence I and a homology arm sequence II capable of complementary pairing to each other to form a homology arm double-stranded region.

In some embodiments, the homology arm sequence I is upstream of the 3′ self-splicing intron fragment and the homology arm sequence II is downstream of the 5′ self-splicing intron fragment.

In some embodiments, the homology arm sequence I or the homology arm sequence II is about 5-200 nucleotides in length, about 5-150 nucleotides in length, about 5-100 nucleotides in length, about 5-80 nucleotides in length, about 5-50 nucleotides in length, about 5-40 nucleotides in length, about 5-30 nucleotides in length, about 5-20 nucleotides in length, about 5-10 nucleotides in length, preferably, about 5, 10, 15, 20, 25, 30, 35 or 40 nucleotides in length.

In some embodiments, one of the homology arm sequence I and the homology arm sequence II comprises a nucleotide sequence of any one of SEQ ID NO: 83-84 or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 83-84, while the other homology arm sequence comprises the corresponding complementary sequence.

In some embodiments, the translation initiation element is an internal ribosome entry site (IRES) which is selected from CVB3, HRV-B3, BRAV-1_L, PV1_L, CAV2_L, BRAV-1, PV1, or CAV2, preferably CVB3.

In embodiments, the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to one of SEQ ID NOs: 120-526.

In some embodiments, the IRES comprises a nucleotide sequence of SEQ ID NO: 121, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 121.

In some embodiments, the at least one protein-coding sequence comprises an AQP-1 protein coding sequence, or a nucleotide sequence encoding a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.

In some embodiments, an AQP-1 protein encoding sequence comprises a nucleotide sequence of SEQ ID NO: 118, or a nucleotide sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 118. In particular, the AQP-1 protein coding sequence is SEQ ID NO: 118, or a nucleotide sequence having 90%, 91%, 92%, 93%, 94%, 95%, 96%, 97%, 98% or 99% sequence identity to SEQ ID NO: 118.

In some embodiments, the circular RNA precursor of the invention comprises a nucleotide sequence selected from any one of SEQ ID NO: 533-536, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 533-536.

In embodiments, the present invention provides a circular RNA precursor comprising from 5′ to 3′ direction:
a) a 3′ self-splicing intron fragment;
b) a non-translation initiation element (non-TIE) fragment II;
c) a nucleotide sequence that encodes an aquaporin-1 (AQP-1) protein, or a variant thereof,
d) a non-translation initiation element (non-TIE) fragment I and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the non-TIE fragment I is derived from a 5′ terminal portion of a non-TIE sequence, and the non-TIE II is derived from a 3′ terminal portion of a non-TIE sequence, and
the non-TIE fragment I comprises a ribozyme recognition site I located at its 3′ end, the non-TIE fragment II comprises a ribozyme recognition site II located at its 5′ end, wherein in the generated circular RNA the ribozyme recognition site I is connected with the ribozyme recognition site II, and thus the non-TIE fragment I is connected with the non-TIE fragment II to form the non-TIE sequence. In embodiments, the non-TIE sequence is a 3′ UTR sequence.

Additionally, the present invention further provides the following items:
1. A circular RNA precursor comprising from 5′ to 3′ direction:
a) a 3′ self-splicing intron fragment; and
b) a 5′ self-splicing intron fragment;
wherein the 3′ self-splicing intron fragment and the 5′ self-splicing intron fragment in combination retain the self-splicing activity of a self-splicing intron and allow generation of a circular RNA through the self-splicing of the circular RNA precursor,
wherein the self-splicing intron is selected from Group I introns and Group II introns, for example, the self-splicing intron is selected from Group I introns.
2. The circular RNA precursor of item 1, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At), for example, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
3. The circular RNA precursor of item 1 or 2, wherein the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.
4. The circular RNA precursor of any one of items 1-3, wherein when the self-splicing intron is cyanobacterium Anabaena Group I Intron such as AnaX, the intron internal split site II and the intron internal split site I are a nucleotide A at position 115 and a nucleotide A at position 116 from 5′ end of the AnaX intron (SEQ ID NO:1) respectively, and when connected form a nucleotide sequence of AA.
5. The circular RNA precursor of any one of items 1-4, wherein when the self-splicing intron is Azoarcus Group I Intron (Azo) (SEQ ID NO:2),
1) the intron internal split site II and the intron internal split site I are a nucleotide A at position 24 and a nucleotide A at position 25 from 5′ end of the Azo intron respectively, and when connected form a nucleotide sequence of AA (P2);
2) the intron internal split site II and the intron internal split site I are a nucleotide A at position 85 and a nucleotide A at position 86 from 5′ end of the Azo intron respectively, and when connected form a nucleotide sequence of AA (P4-5);
3) the intron internal split site II and the intron internal split site I are a nucleotide C at position 71 and a nucleotide C at position 72 from 5′ end of the Azo intron respectively, and when connected form a nucleotide sequence of CC (P5);
4) the intron internal split site II and the intron internal split site I are a nucleotide C at position 108 and a nucleotide U at position 109 from 5′ end of the Azo intron respectively, and when connected form a nucleotide sequence of CU (P6);
5) the intron internal split site II and the intron internal split site I are a nucleotide A at position 154 and a nucleotide A at position 155 from 5′ end of the Azo intron respectively, and when connected form a nucleotide sequence of AA (P8); or
6) the intron internal split site II and the intron internal split site I are a nucleotide C at position 198 and a nucleotide A at position 199 from 5′ end of the Azo intron respectively, and when connected form a nucleotide sequence of CA (P9).
6. The circular RNA precursor of any one of items 1-4, wherein when the self-splicing intron is Scytalidium dimidiatum Group I Intron (Sd) (SEQ ID NO.4),
1) the intron internal split site II and the intron internal split site I are a nucleotide A at position 88 and a nucleotide G at position 89 from 5′ end of the Sd intron respectively, and when connected form a nucleotide sequence of AG (P2);
2) the intron internal split site II and the intron internal split site I are a nucleotide U at position 173 and a nucleotide G at position 174 from 5′ end of the Sd intron respectively, and when connected form a nucleotide sequence of UG (P5);
3) the intron internal split site II and the intron internal split site I are a nucleotide A at position 204 and a nucleotide G at position 205 from 5′ end of the Sd intron respectively, and when connected form a nucleotide sequence of AG (P6);
4) the intron internal split site II and the intron internal split site I are a nucleotide C at position 252 and a nucleotide G at position 253 from 5′ end of the Sd intron respectively, and when connected form a nucleotide sequence of CG (P8); or
5) the intron internal split site II and the intron internal split site I are a nucleotide C at position 370 and a nucleotide G at position 371 from 5′ end of the Sd intron respectively, and when connected form a nucleotide sequence of CG (P9).
7. The circular RNA precursor of any one of items 1-4, wherein when the self-splicing intron is Staphylococcus phage Twort Group I Intron (Twort) (SEQ ID NO:3),
1) the intron internal split site II and the intron internal split site I are a nucleotide U at position 70 and a nucleotide A at position 71 from 5′ end of the Twort intron respectively, and when connected form a nucleotide sequence of UA (P5);
2) the intron internal split site II and the intron internal split site I are a nucleotide U at position 103 and a nucleotide A at position 104 from 5′ end of the Twort intron respectively, and when connected form a nucleotide sequence of UA (P6);
3) the intron internal split site II and the intron internal split site I are a nucleotide A at position 132 and a nucleotide U at position 133 from 5′ end of the Twort intron respectively, and when connected form a nucleotide sequence of AU (P7);
4) the intron internal split site II and the intron internal split site I are a nucleotide U at position 173 and a nucleotide C at position 174 from 5′ end of the Twort intron respectively, and when connected form a nucleotide sequence of UC (P8);
5) the intron internal split site II and the intron internal split site I are a nucleotide A at position 206 and a nucleotide A at position 207 from 5′ end of the Twort intron respectively, and when connected form a nucleotide sequence of AA (P9);
6) the intron internal split site II and the intron internal split site I are a nucleotide G at position 226 and a nucleotide U at position 227 from 5′ end of the Twort intron respectively, and when connected form a nucleotide sequence of GU (P9.1);
7) the intron internal split site II and the intron internal split site I are a nucleotide U at position 23 and a nucleotide A at position 24 from 5′ end of the Twort intron respectively, and when connected form a nucleotide sequence of UA (P2); or
8) the intron internal split site II and the intron internal split site I are a nucleotide A at position 82 and a nucleotide A at position 83 from 5′ end of the Twort intron respectively, and when connected form a nucleotide sequence of AA (P4-5).
8. The circular RNA precursor of any one of items 1-4, wherein when the self-splicing intron is Scytonema-hofmani tRNA fMet group I intron (Sh) (SEQ ID NO:5),
1) the intron internal split site II and the intron internal split site I are a nucleotide A at position 129 and a nucleotide A at position 130 from 5′ end of the Sh intron respectively, and when connected form a nucleotide sequence of AA (P6);
2) the intron internal split site II and the intron internal split site I are a nucleotide A at position 60 and a nucleotide A at position 61 from 5′ end of the Sh intron respectively, and when connected form a nucleotide sequence of AA (P2);
3) the intron internal split site II and the intron internal split site I are a nucleotide A at position 109 and a nucleotide A at position 110 from 5′ end of the Sh intron respectively, and when connected form a nucleotide sequence of AA (P4-5); or
4) the intron internal split site II and the intron internal split site I are a nucleotide A at position 213 and a nucleotide A at position 214 from 5′ end of the Sh intron respectively, and when connected form a nucleotide sequence of UA (P9).
9. The circular RNA precursor of any one of items 1-4, wherein when the self-splicing intron is Agrobacterium-tumefaciens group I intron (At) (SEQ ID NO:6), the intron internal split site II and the intron internal split site I are a nucleotide A at position 107 and a nucleotide A at position 108 from 5′ end of the At intron respectively, and when connected form a nucleotide sequence of AA (P6).
10. The circular RNA precursor of any one of items 1-9, wherein the 3′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 3′ terminal portion of the self-splicing intron, and the 5′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 5′ terminal portion of the self-splicing intron.
11. The circular RNA precursor of any one of items 1-10, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
12. The circular RNA precursor of any one of items 1-10, wherein the self-splicing intron is the Azoarcus Group I Intron (Azo), the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.
13. The circular RNA precursor of any one of items 1-10, wherein the self-splicing intron is the Scytalidium dimidiatum Group I Intron (Sd), the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.
14. The circular RNA precursor of any one of items 1-10, wherein the self-splicing intron is the Staphylococcus phage Twort Group I Intron (Twort), the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.
15. The circular RNA precursor of any one of items 1-10, wherein the self-splicing intron is the Scytonema-hofmani tRNA fMet group I intron (Sh), the 3′ self-splicing intron fragment comprises or consists of a nucleotide sequence of SEQ ID NO: 47, 49, 51 or 53 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51 or 53 and the 5′ self-splicing intron fragment comprises or consists of a nucleotide sequence of SEQ ID NO: 48, 50, 52 or 54 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52 or 54.
16. The circular RNA precursor of any one of items 1-10, wherein the self-splicing intron is the Agrobacterium-tumefaciens group I intron (At), the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.
17. The circular RNA precursor of any one of items 1-16, which further comprises from 5′ to 3′ direction: optionally a first residual circularizing element; a nucleotide sequence of interest; and optionally a second residual circularizing element, located between a) and b), wherein the total length of the first residual circularizing element and the second residual circularizing element is about 0 to about 500 nucleotides; or
the circular RNA precursor further comprises a homology arm sequence I upstream of the 3′ self-splicing intron fragment and a homology arm sequence II downstream of the 5′ self-splicing intron fragment.
18. The circular RNA precursor of any one of items 1-17, wherein the first residual circularizing element comprises or consists of from 5′ to 3′ direction a 3′ exon region and optionally a first spacer, the second residual circularizing element comprises or consists of from 3′ to 5′ direction a 5′ exon region and optionally a second spacer.
19. The circular RNA precursor of item 18, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
20. The circular RNA precursor of item 19, wherein 3′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the native 5′ exon or a contiguous fragment of about 1-about 50 nucleotides starting from the 3′ terminal nucleotide of the native 5′ exon.
21. A circular RNA precursor comprising from 5′ to 3′ direction:
a) a 3′ self-splicing intron fragment;
b) a first residual circularizing element;
c) a nucleotide sequence of interest;
d) a second residual circularizing element; and
e) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the first residual circularizing element, the nucleotide sequence of interest, and the second residual circularizing element through the self-splicing of the circular RNA precursor,
wherein the total length of the first residual circularizing element and the second residual circularizing element is about 0 to about 500 nucleotides.
22. The circular RNA precursor of item 21, wherein the self-splicing intron is selected from Group I introns and Group II introns, for example, the self-splicing intron is selected from Group I introns.
23. The circular RNA precursor of item 21 or 22, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium- tumefaciens group I intron (At), for example, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
24. The circular RNA precursor of any one of items 21-23, wherein the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.
25. The circular RNA precursor of any one of items 21-23, wherein when the self-splicing intron is cyanobacterium Anabaena Group I Intron such as AnaX, the intron internal split site II and the intron internal split site I are a nucleotide A at position 115 and a nucleotide A at position 116 from 5′ end of the AnaX intron (SEQ ID NO:1) respectively, and when connected form a nucleotide sequence of AA.
26. The circular RNA precursor of any one of items 21-24, wherein when the self-splicing intron is Azoarcus Group I Intron (Azo) (SEQ ID NO:2),
1) the intron internal split site II and the intron internal split site I are a nucleotide A at position 24 and a nucleotide A at position 25 from 5′ end of the Azo intron respectively, and when connected form a nucleotide sequence of AA (P2);
2) the intron internal split site II and the intron internal split site I are a nucleotide A at position 85 and a nucleotide A at position 86 from 5′ end of the Azo intron respectively, and when connected form a nucleotide sequence of AA (P4-5);
3) the intron internal split site II and the intron internal split site I are a nucleotide C at position 71 and a nucleotide C at position 72 from 5′ end of the Azo intron respectively, and when connected form a nucleotide sequence of CC (P5);
4) the intron internal split site II and the intron internal split site I are a nucleotide C at position 108 and a nucleotide U at position 109 from 5′ end of the Azo intron respectively, and when connected form a nucleotide sequence of CU (P6);
5) the intron internal split site II and the intron internal split site I are a nucleotide A at position 154 and a nucleotide A at position 155 from 5′ end of the Azo intron respectively, and when connected form a nucleotide sequence of AA (P8); or
6) the intron internal split site II and the intron internal split site I are a nucleotide C at position 198 and a nucleotide A at position 199 from 5′ end of the Azo intron respectively, and when connected form a nucleotide sequence of CA (P9).
27. The circular RNA precursor of any one of items 21-24, wherein when the self-splicing intron is Scytalidium dimidiatum Group I Intron (Sd) (SEQ ID NO:4),
1) the intron internal split site II and the intron internal split site I are a nucleotide A at position 88 and a nucleotide G at position 89 from 5′ end of the Sd intron respectively, and when connected form a nucleotide sequence of AG (P2);
2) the intron internal split site II and the intron internal split site I are a nucleotide U at position 173 and a nucleotide G at position 174 from 5′ end of the Sd intron respectively, and when connected form a nucleotide sequence of UG (P5);
3) the intron internal split site II and the intron internal split site I are a nucleotide A at position 204 and a nucleotide G at position 205 from 5′ end of the Sd intron respectively, and when connected form a nucleotide sequence of AG (P6);
4) the intron internal split site II and the intron internal split site I are a nucleotide C at position 252 and a nucleotide G at position 253 from 5′ end of the Sd intron respectively, and when connected form a nucleotide sequence of CG (P8); or
5) the intron internal split site II and the intron internal split site I are a nucleotide C at position 370 and a nucleotide G at position 371 from 5′ end of the Sd intron respectively, and when connected form a nucleotide sequence of CG (P9).
28. The circular RNA precursor of any one of items 21-24, wherein when the self-splicing intron is Staphylococcus phage Twort Group I Intron (Twort) (SEQ ID NO:3),
1) the intron internal split site II and the intron internal split site I are a nucleotide U at position 70 and a nucleotide A at position 71 from 5′ end of the Twort intron respectively, and when connected form a nucleotide sequence of UA (P5);
2) the intron internal split site II and the intron internal split site I are a nucleotide U at position 103 and a nucleotide A at position 104 from 5′ end of the Twort intron respectively, and when connected form a nucleotide sequence of UA (P6);
3) the intron internal split site II and the intron internal split site I are a nucleotide A at position 132 and a nucleotide U at position 133 from 5′ end of the Twort intron respectively, and when connected form a nucleotide sequence of AU (P7);
4) the intron internal split site II and the intron internal split site I are a nucleotide U at position 173 and a nucleotide C at position 174 from 5′ end of the Twort intron respectively, and when connected form a nucleotide sequence of UC (P8);
5) the intron internal split site II and the intron internal split site I are a nucleotide A at position 206 and a nucleotide A at position 207 from 5′ end of the Twort intron respectively, and when connected form a nucleotide sequence of AA (P9);
6) the intron internal split site II and the intron internal split site I are a nucleotide G at position 226 and a nucleotide U at position 227 from 5′ end of the Twort intron respectively, and when connected form a nucleotide sequence of GU (P9.1);
7) the intron internal split site II and the intron internal split site I are a nucleotide U at position 23 and a nucleotide A at position 24 from 5′ end of the Twort intron respectively, and when connected form a nucleotide sequence of UA (P2); or
8) the intron internal split site II and the intron internal split site I are a nucleotide A at position 82 and a nucleotide A at position 83 from 5′ end of the Twort intron respectively, and when connected form a nucleotide sequence of AA (P4-5).
29. The circular RNA precursor of any one of items 21-24, wherein when the self-splicing intron is Scytonema-hofmani tRNA fMet group I intron (Sh) (SEQ ID NO:5),
1) the intron internal split site II and the intron internal split site I are a nucleotide A at position 129 and a nucleotide A at position 130 from 5′ end of the Sh intron respectively, and when connected form a nucleotide sequence of AA (P6);
2) the intron internal split site II and the intron internal split site I are a nucleotide A at position 60 and a nucleotide A at position 61 from 5′ end of the Sh intron respectively, and when connected form a nucleotide sequence of AA (P2);
3) the intron internal split site II and the intron internal split site I are a nucleotide A at position 109 and a nucleotide A at position 110 from 5′ end of the Sh intron respectively, and when connected form a nucleotide sequence of AA (P4-5); or
4) the intron internal split site II and the intron internal split site I are a nucleotide A at position 213 and a nucleotide A at position 214 from 5′ end of the Sh intron respectively, and when connected form a nucleotide sequence of UA (P9).
30. The circular RNA precursor of any one of items 21-24, wherein when the self-splicing intron is Agrobacterium-tumefaciens group I intron (At) (SEQ ID NO:6), the intron internal split site II and the intron internal split site I are a nucleotide A at position 107 and a nucleotide A at position 108 from 5′ end of the At intron respectively, and when connected form a nucleotide sequence of AA (P6).
31. The circular RNA precursor of any one of items 21-30, wherein the 3′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 3′ terminal portion of the self-splicing intron, and the 5′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 5′ terminal portion of the self-splicing intron.
32. The circular RNA precursor of any one of items 21-31, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
33. The circular RNA precursor of any one of items 21-31, wherein the self-splicing intron is the Azoarcus Group I Intron (Azo), the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.
34. The circular RNA precursor of any one of items 21-31, wherein the self-splicing intron is the Scytalidium dimidiatum Group I Intron (Sd), the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.
35. The circular RNA precursor of any one of items 21-31, wherein the self-splicing intron is the Staphylococcus phage Twort Group I Intron (Twort), the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.
36. The circular RNA precursor of any one of items 21-31, wherein the self-splicing intron is the Scytonema-hofmani tRNA fMet group I intron (Sh), the 3′ self-splicing intron fragment comprises or consists of a nucleotide sequence of SEQ ID NO: 47, 49, 51 or 53 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51 or 53, and the 5′ self-splicing intron fragment comprises or consists of a nucleotide sequence of SEQ ID NO: 48, 50, 52 or 54 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52 or 54.
37. The circular RNA precursor of any one of items 21-31, wherein the self-splicing intron is the Agrobacterium-tumefaciens group I intron (At), the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.
38. The circular RNA precursor of any one of items 21-37, wherein the first residual circularizing element and the second residual circularizing element are configured such that the circular RNA comprising them has reduced immunogenicity.
39. The circular RNA precursor of any one of items 21-38, wherein the first residual circularizing element and the second residual circularizing element are configured such that the circular RNA precursor comprising them has comparable or increased circularization efficiency.
40. The circular RNA precursor of any one of items 21-39, wherein the total length of the first residual circularizing element and the second residual circularizing element is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides.
41. The circular RNA precursor of any one of items 21-40, wherein the first residual circularizing element and the second residual circularizing element are configured to be capable of forming a stem-loop structure, e.g., upon self-splicing for circularization.
42. The circular RNA precursor of item 41, wherein the loop of the stem-loop structure comprises the splicing junction.
43. The circular RNA precursor of any one of items 41-42, wherein the first residual circularizing element comprises the sequence structure of the following formula: 5′-first loop sequence-first pairing sequence-first non-pairing sequence-3′; and the second residual circularizing element comprises the sequence structure of the following formula: 5′-second non-pairing sequence-second pairing sequence-second loop sequence-3′,
wherein the first non-pairing sequence or the second non-pairing sequence may be independently present or absent,
the first pairing sequence and the second pairing sequence can complementarily pair to each other to form the stem of the stem-loop structure, wherein the first loop sequence and the second loop sequence can form the loop of the stem-loop structure, e.g., through self-splicing for circularization.
44. The circular RNA precursor of any one of items 41-43, wherein the first residual circularizing element can be selected from various sequence, provided that the first loop sequence of the first residual circularizing element doesn't pair with any sequence of the second residual circularizing element.
45. The circular RNA precursor of item 43 or 44, wherein the first loop sequence comprises or consists of one or more nucleotides which can pair with the P1 region of the corresponding self-splicing intron to form a P10 duplex region during the circularization.
46. The circular RNA precursor of any one of items 43-45, wherein the first loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.
47. The circular RNA precursor of any one of items 43-46, wherein the first loop sequence comprises or consists of the sequence of AAAA, AGAA, AA, UUUU, UAAA, CAAA, or GAAA, preferably AAAA.
48. The circular RNA precursor of any one of items 43-47, wherein the second loop sequence comprises or consists of one or more nucleotides which can pair with the internal guide sequence (IGS) of the corresponding self-splicing intron to form a P1 duplex region during the circularization.
49. The circular RNA precursor of any one of items 43-48, wherein the second loop sequence comprises or consists of a nucleotide sequence of any one of SEQ ID NO: 95-116, such as CUU, CAU or CUC, preferably CUU.
50. The circular RNA precursor of any one of items 43-49, wherein the loop of the stem-loop structure has a sequence of CUUAAAA, CUUUTUUU, CUUAA, CUUGAAA, CUUUAAA, CUUAGAA, CUUCAAA or CUCAAAA, preferably CUUAAAA.
51. The circular RNA precursor of any one of items 43-50, wherein the stem portion of the stem-loop structure comprises 2-15 or more consecutive matched base pairs, preferably, the stem portion of the stem-loop structure comprises 5, 6 or 7 consecutive matched base pairs.
52. The circular RNA precursor of any one of items 43-51, wherein the stem portion in the stem-loop structure comprises up to 2 base pair mismatches, or the stem portion comprises only 1 base pair mismatch, preferably, the stem portion comprises no base pair mismatches.
53. The circular RNA precursor of any one of items 43-52, wherein the first pairing sequence comprises only G and the second pairing sequence comprises only C, or the first pairing sequence comprises only C and the second pairing sequence comprises only G.
54. The circular RNA precursor of any one of items 43-53, wherein the first pairing sequence comprises only A and the second pairing sequence comprises only U.
55. The circular RNA precursor of any one of items 43-54, wherein the first pairing sequence comprises only U and the second pairing sequence comprises only A.
56. The circular RNA precursor of any one of items 21-55, wherein the first residual circularizing element comprises or consists of from 5′ to 3′ direction a 3′ exon region and optionally a first spacer, the second residual circularizing element comprises or consists of from 3′ to 5′ direction a 5′ exon region and optionally a second spacer.
57. The circular RNA precursor of item 56, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
58. The circular RNA precursor of item 56 or 57, wherein the 3′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 3′ exon or a contiguous fragment of about 1-about 50 nucleotides starting from the 5′ terminal nucleotide of the native 3′ exon,
the 5′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 5′ exon or a contiguous fragment of about 1-about 50 nucleotides starting from the 3′ terminal nucleotide of the native 5′ exon.
59. The circular RNA precursor of any one of items 21-58, wherein the first residual circularizing element comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69, preferably the first residual circularizing element is selected from a nucleotide sequence of AAAAUCCGUUGA or AAAACCCCCCCA.
60. The circular RNA precursor of any one of items 21-59, wherein the second residual circularizing element comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70, preferably the second residual circularizing element is selected from a nucleotide sequence of AGACGCUACGGACUU orAGACGGGGGGGGCUU.
61. The circular RNA precursor of any one of items 21-60, wherein the first residual circularizing element comprises or consists of a nucleotide sequence of any one of SEQ ID NO: 57, 59, 61, 63, 65, 67 or 69 and the second residual circularizing element comprises or consists of a nucleotide sequence of any one of SEQ ID NO: 58, 60, 62, 64, 66, 68 or 70.
62. The circular RNA precursor of any one of items 21-61, wherein,
1) the first residual circularizing element comprises or consists of a nucleotide sequence of SEQ ID NO: 57 and the second residual circularizing element comprises or consists of a nucleotide sequence of SEQ ID NO: 58;
2) the first residual circularizing element comprises or consists of a nucleotide sequence of SEQ ID NO: 59 and the second residual circularizing element comprises or consists of a nucleotide sequence of SEQ ID NO: 60;
3) the first residual circularizing element comprises or consists of a nucleotide sequence of SEQ ID NO: 61 and the second residual circularizing element comprises or consists of a nucleotide sequence of SEQ ID NO: 62;
4) the first residual circularizing element comprises or consists of a nucleotide sequence of SEQ ID NO: 63 and the second residual circularizing element comprises or consists of a nucleotide sequence of SEQ ID NO: 64;
5) the first residual circularizing element comprises or consists of a nucleotide sequence of SEQ ID NO: 65 and the second residual circularizing element comprises or consists of a nucleotide sequence of SEQ ID NO: 66;
6) the first residual circularizing element comprises or consists of a nucleotide sequence of SEQ ID NO: 67 and the second residual circularizing element comprises or consists of a nucleotide sequence of SEQ ID NO: 68; or
7) the first residual circularizing element comprises or consists of a nucleotide sequence of SEQ ID NO: 69 and the second residual circularizing element comprises or consists of a nucleotide sequence of SEQ ID NO: 70;
preferably, the first residual circularizing element is selected from a nucleotide sequence of AAAAUCCGUUGA and the second residual circularizing element is selected from a nucleotide sequence of AGACGCUACGGACUU, or the first residual circularizing element is selected from a nucleotide sequence of AAAACCCCCCCA and the second residual circularizing element is selected from a nucleotide sequence of AGACGGGGGGGGCUU.
63. The circular RNA precursor of any one of items 21-62, wherein the circular RNA precursor further comprises a homology arm sequence I and a homology arm sequence II capable of complementary pairing to each other to form a homology arm double-stranded region.
64. The circular RNA precursor of item 63, wherein the homology arm sequence I is upstream of the 3′ self-splicing intron fragment and the homology arm sequence II is downstream of the 5′ self-splicing intron fragment.
65. The circular RNA precursor of any one of items 61-62, wherein the homology arm sequence I or the homology arm sequence II is about 5-500, about 5-450, about 5-400, about 5-350, about 5-300, about 5-250, about 5-200, about 5-150, about 5-100, about 5-80, about 5-60, about 5-50, about 5-40, about 5-30, about 5-25, about 5-20, about 5-15 or about 5-10 nucleotides in length, preferably, about 20, 25, 40, 50, 100, or 300 nucleotides in length.
66. The circular RNA precursor of any one of items 61-65, wherein the two homology arm sequences may be polyA and polyU, respectively, or polyG and polyC, respectively.
67. The circular RNA precursor of any one of items 61-66, wherein one of the homology arm sequence I and the homology arm sequence II comprises a nucleotide sequence of any one of SEQ ID NO: 71-94 or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94, while the other homology arm sequence comprises the corresponding complementary sequence.
68. The circular RNA precursor of any one of items 21-67, wherein the nucleotide sequence of interest comprises at least one protein-coding sequence and a translation initiation element such as an internal ribosome entry site (IRES) operably linked thereto.
69. The circular RNA precursor of item 68, wherein the translation initiation element, such as an IRES, is located upstream to the 5′ end of the at least one protein-coding sequence.
70. The circular RNA precursor of item 68, wherein the translation initiation element, such as an IRES, is located downstream to the 3′ end of the at least one protein-coding sequence.
71. The circular RNA precursor of any one of items 68-70, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is selected from CVB3, HRV-B3, BRAV-1_L, PV1_L, CAV2_L, BRAV-1, PV1, or CAV2.
72. The circular RNA precursor of any one of items 68-71, wherein the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 120-526.
73. The circular RNA precursor of any one of items 68-72, wherein the at least one protein-coding sequence encodes a protein of eukaryotic, prokaryotic or viral origin, for example, a protein for therapeutic or diagnostic use;
preferably, the at least one protein-coding sequence encodes IL-2, POLR2A, or an aquaporin, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
74. The circular RNA precursor of any one of items 68-73, wherein the at least one protein-coding sequence comprises a nucleotide sequence of any one of SEQ ID NO: 117-118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.
75. The circular RNA precursor of any one of items 68-74, wherein the nucleotide sequence of interest is a non-coding sequence, for example, the non-coding sequence can be antisense RNA, aptamer, guide RNA, or non-protein-coding RNA existing in any organism.
76. The circular RNA precursor of any one of items 21-75, which comprises a nucleotide sequence selected from any one of SEQ ID NO: 527, 532 and 537-542, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 527, 532 and 537-542.
77. A circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a circularizing sequence fragment II comprising a ribozyme recognition site II at its 5′ end and a circularizing sequence fragment I comprising a ribozyme recognition site I at its 3′ end,
wherein the circularizing sequence fragment I is derived from a 5′ terminal portion of a circularizing sequence, and the circularizing sequence fragment II is derived from a 3′ terminal portion of a circularizing sequence, and
wherein in the generated circular RNA the ribozyme recognition site I is connected with the ribozyme recognition site II, and thus the circularizing sequence fragment I is connected with the circularizing sequence fragment II to form the circularizing sequence.
78. The circular RNA precursor of item 77, wherein the nucleotide sequence of interest comprises one or more sequences selected from the following group between the circularizing sequence fragment II and the circularizing sequence fragment I: at least one protein coding sequences, a non-coding sequence, translation initiation element (TIE), a non-TIE functional element or any combination thereof.
79. The circular RNA precursor of item 77 or 78, wherein the circularizing sequence is selected from at least one protein-coding sequence, a non-coding sequence, a translation initiation element (TIE), or a non-TIE functional element.
80. A circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is derived from a 5′ terminal portion of at least one protein-coding sequence, and the protein-coding sequence fragment II is derived from a 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence;
optionally, the at least one protein-coding sequence comprises at least two protein-coding sequences, and a further translation initiation element, 2A sequence or spacer may be linked between any two adjacent protein-coding sequences, and the translation initiation element comprises: IRES sequence, 5′ UTR sequence, Kozak sequence, a sequence containing m6A, a sequence complementary to ribosome 18S rRNA, or any combination thereof.
81. The circular RNA precursor of any one of items 77-80, wherein the self-splicing intron is selected from the Group I introns and Group II introns, for example, the self-splicing intron is selected from Group I introns.
82. The circular RNA precursor of any one of items 77-81, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA Met group I intron (Sh), or Agrobacterium-tumefaciens group I intron (At), for example, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
83. The circular RNA precursor of any one of items 77-82, wherein the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.
84. The circular RNA precursor of any one of items 77-83, wherein when the self-splicing intron is cyanobacterium Anabaena Group I Intron such as AnaX, the intron internal split site II and the intron internal split site I are a nucleotide A at position 115 and a nucleotide A at position 116 from 5′ end of the AnaX intron (SEQ ID NO:1) respectively, and when connected form a nucleotide sequence of AA.
85. The circular RNA precursor of any one of items 77-83, wherein when the self-splicing intron is Azoarcus Group I Intron (Azo) (SEQ ID NO:2),
1) the intron internal split site II and the intron internal split site I are a nucleotide A at position 24 and a nucleotide A at position 25 from 5′ end of the Azo intron respectively, and when connected form a nucleotide sequence of AA (P2);
2) the intron internal split site II and the intron internal split site I are a nucleotide A at position 85 and a nucleotide A at position 86 from 5′ end of the Azo intron respectively, and when connected form a nucleotide sequence of AA (P4-5);
3) the intron internal split site II and the intron internal split site I are a nucleotide C at position 71 and a nucleotide C at position 72 from 5′ end of the Azo intron respectively, and when connected form a nucleotide sequence of CC (P5);
4) the intron internal split site II and the intron internal split site I are a nucleotide C at position 108 and a nucleotide U at position 109 from 5′ end of the Azo intron respectively, and when connected form a nucleotide sequence of CU (P6);
5) the intron internal split site II and the intron internal split site I are a nucleotide A at position 154 and a nucleotide A at position 155 from 5′ end of the Azo intron respectively, and when connected form a nucleotide sequence of AA (P8); or
6) the intron internal split site II and the intron internal split site I are a nucleotide C at position 198 and a nucleotide A at position 199 from 5′ end of the Azo intron respectively, and when connected form a nucleotide sequence of CA (P9).
86. The circular RNA precursor of any one of items 77-83, wherein when the self-splicing intron is Scytalidium dimidiatum Group I Intron (Sd) (SEQ ID NO:4),
1) the intron internal split site II and the intron internal split site I are a nucleotide A at position 88 and a nucleotide G at position 89 from 5′ end of the Sd intron respectively, and when connected form a nucleotide sequence of AG (P2);
2) the intron internal split site II and the intron internal split site I are a nucleotide U at position 173 and a nucleotide G at position 174 from 5′ end of the Sd intron respectively, and when connected form a nucleotide sequence of UG (P5);
3) the intron internal split site II and the intron internal split site I are a nucleotide A at position 204 and a nucleotide G at position 205 from 5′ end of the Sd intron respectively, and when connected form a nucleotide sequence of AG (P6);
4) the intron internal split site II and the intron internal split site I are a nucleotide C at position 252 and a nucleotide G at position 253 from 5′ end of the Sd intron respectively, and when connected form a nucleotide sequence of CG (P8); or
5) the intron internal split site II and the intron internal split site I are a nucleotide C at position 370 and a nucleotide G at position 371 from 5′ end of the Sd intron respectively, and when connected form a nucleotide sequence of CG (P9).
87. The circular RNA precursor of any one of items 77-83, wherein when the self-splicing intron is Staphylococcus phage Twort Group I Intron (Twort) (SEQ ID NO:3),
1) the intron internal split site II and the intron internal split site I are a nucleotide U at position 70 and a nucleotide A at position 71 from 5′ end of the Twort intron respectively, and when connected form a nucleotide sequence of UA (P5);
2) the intron internal split site II and the intron internal split site I are a nucleotide U at position 103 and a nucleotide A at position 104 from 5′ end of the Twort intron respectively, and when connected form a nucleotide sequence of UA (P6);
3) the intron internal split site II and the intron internal split site I are a nucleotide A at position 132 and a nucleotide U at position 133 from 5′ end of the Twort intron respectively, and when connected form a nucleotide sequence of AU (P7);
4) the intron internal split site II and the intron internal split site I are a nucleotide U at position 173 and a nucleotide C at position 174 from 5′ end of the Twort intron respectively, and when connected form a nucleotide sequence of UC (P8);
5) the intron internal split site II and the intron internal split site I are a nucleotide A at position 206 and a nucleotide A at position 207 from 5′ end of the Twort intron respectively, and when connected form a nucleotide sequence of AA (P9);
6) the intron internal split site II and the intron internal split site I are a nucleotide G at position 226 and a nucleotide U at position 227 from 5′ end of the Twort intron respectively, and when connected form a nucleotide sequence of GU (P9.1);
7) the intron internal split site II and the intron internal split site I are a nucleotide U at position 23 and a nucleotide A at position 24 from 5′ end of the Twort intron respectively, and when connected form a nucleotide sequence of UA (P2); or
8) the intron internal split site II and the intron internal split site I are a nucleotide A at position 82 and a nucleotide A at position 83 from 5′ end of the Twort intron respectively, and when connected form a nucleotide sequence of AA (P4-5).
88. The circular RNA precursor of any one of items 77-83, wherein when the self-splicing intron is Scytonema-hofmani tRNA fMet group I intron (Sh) (SEQ ID NO:5),
1) the intron internal split site II and the intron internal split site I are a nucleotide A at position 129 and a nucleotide A at position 130 from 5′ end of the Sh intron respectively, and when connected form a nucleotide sequence of AA (P6);
2) the intron internal split site II and the intron internal split site I are a nucleotide A at position 60 and a nucleotide A at position 61 from 5′ end of the Sh intron respectively, and when connected form a nucleotide sequence of AA (P2);
3) the intron internal split site II and the intron internal split site I are a nucleotide A at position 109 and a nucleotide A at position 110 from 5′ end of the Sh intron respectively, and when connected form a nucleotide sequence of AA (P4-5); or
4) the intron internal split site II and the intron internal split site I are a nucleotide A at position 213 and a nucleotide A at position 214 from 5′ end of the Sh intron respectively, and when connected form a nucleotide sequence of UA (P9).
89. The circular RNA precursor of any one of items 77-83, wherein when the self-splicing intron is Agrobacterium-tumefaciens group I intron (At) (SEQ ID NO:6), the intron internal split site II and the intron internal split site I are a nucleotide A at position 107 and a nucleotide A at position 108 from 5′ end of the At intron respectively, and when connected form a nucleotide sequence of AA (P6).
90. The circular RNA precursor of any one of items 77-89, wherein the 3′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 3′ terminal portion of the self-splicing intron, and the 5′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 5′ terminal portion of the self-splicing intron.
91. The circular RNA precursor of any one of items 77-90, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
92. The circular RNA precursor of any one of items 77-90, wherein the self-splicing intron is the Azoarcus Group I Intron (Azo), the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.
93. The circular RNA precursor of any one of items 77-90, wherein the self-splicing intron is the Scytalidium dimidiatum Group I Intron (Sd), the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.
94. The circular RNA precursor of any one of items 77-90, wherein the self-splicing intron is the Staphylococcus phage Twort Group I Intron (Twort), the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.
95. The circular RNA precursor of any one of items 77-90, wherein the self-splicing intron is the Scytonema-hofmani tRNA fMet group I intron (Sh), the 3′ self-splicing intron fragment comprises or consists of a nucleotide sequence of SEQ ID NO: 47, 49, 51 or 53 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51 or 53, and the 5′ self-splicing intron fragment comprises or consists of a nucleotide sequence of SEQ ID NO: 48, 50, 52 or 54 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52 or 54.
96. The circular RNA precursor of any one of items 77-90, wherein the self-splicing intron is the Agrobacterium-tumefaciens group I intron (At), the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.
97. The circular RNA precursor of any one of items 77-96, wherein the ribozyme recognition site I and the ribozyme recognition site II are configured such that the circular RNA comprising them has reduced immunogenicity.
98. The circular RNA precursor of any one of items 77-97, wherein the ribozyme recognition site I and the ribozyme recognition site II are configured such that the circular RNA precursor comprising them has comparable or increased circularization efficiency.
99. The circular RNA precursor of any one of items 77-98, wherein the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.
100. The circular RNA precursor of any one of items 77-99, wherein the total length of the ribozyme recognition site I is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.
101. The circular RNA precursor of any one of items 77-100, wherein the ribozyme recognition site I and the ribozyme recognition site II are configured to be capable of forming a stem-loop structure, e.g., upon self-splicing for circularization.
102. The circular RNA precursor of item 101, wherein the loop of the stem-loop structure comprises the splicing junction.
103. The circular RNA precursor of any one of items 77-102, wherein the ribozyme recognition site I comprises the sequence structure of the following formula: 5′-third non-pairing sequence-third pairing sequence-third loop sequence-3′; and the ribozyme recognition site II comprises the sequence structure of the following formula: 5′-fourth loop sequence-fourth pairing sequence-fourth non-pairing sequence-3′,
wherein the third non-pairing sequence or the fourth non-pairing sequence may be independently present or absent,
the third pairing sequence and the fourth pairing sequence can complementarily pair to each other to form the stem of the stem-loop structure, wherein the third loop sequence and the fourth loop sequence can form the loop of the stem-loop structure, e.g., through self-splicing for circularization.
104. The circular RNA precursor of any one of items 77-103, wherein the ribozyme recognition site II can be selected from various sequence.
105. The circular RNA precursor of item 103 or 104, wherein the fourth loop sequence comprises or consists of one or more nucleotides which can pair with the P1 region of the corresponding self-splicing intron to form a P10 duplex region during the circularization.
106. The circular RNA precursor of any one of items 103-105, wherein the fourth loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or20.
107. The circular RNA precursor of any one of items 103-106, wherein the fourth loop sequence comprises or consists of the sequence of AAAA, AGAA, AA, UUUU, UAAA, CAAA, or GAAA, preferably AAAA.
108. The circular RNA precursor of any one of items 103-107, wherein the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.
109. The circular RNA precursor of any one of items 103-108, wherein the third loop sequence comprises or consists of one or more nucleotides which can pair with the internal guide sequence (IGS) of the corresponding self-splicing intron to form a P1 duplex region during the circularization.
110. The circular RNA precursor of any one of items 103-109, wherein the third loop sequence comprises or consists of a nucleotide sequence of any one of SEQ ID NO: 95-116, such as CUU, CAU or CUC, preferably CUU.
111. The circular RNA precursor of any one of items 103-110, wherein the loop of the stem-loop structure has a sequence of CUUAAAA, CUUUUUU, CUUAA, CUUGAAA, CUUUAAA, CUUAGAA, CUUCAAA or CUCAAAA, preferably CUUAAAA.
112. The circular RNA precursor of any one of items 103-111, wherein the stem portion of the stem-loop structure comprises 2-15 or more consecutive matched base pairs, preferably, the stem portion of the stem-loop structure comprises 5, 6 or 7 consecutive matched base pairs.
113. The circular RNA precursor of any one of items 103-112, wherein the stem portion in the stem-loop structure comprises up to 2 base pair mismatches, or the stem portion comprises only 1 base pair mismatch, preferably, the stem portion comprises no base pair mismatches.
114. The circular RNA precursor of any one of items 103-113, wherein the third pairing sequence comprises only G and the fourth pairing sequence comprises only C, or the third pairing sequence comprises only C and the fourth pairing sequence comprises only G.
115. The circular RNA precursor of any one of items 103-114, wherein the third pairing sequence comprises only A and the fourth pairing sequence comprises only U, or the third pairing sequence comprises only U and the fourth pairing sequence comprises only A.
116. The circular RNA precursor of any one of items 77-115, wherein the ribozyme recognition site II comprises or consists of a 3′ exon region, and the ribozyme recognition site I comprises or consists of a 5′ exon region.
117. The circular RNA precursor of item 116, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
118. The circular RNA precursor of item 116 or 117, wherein the 3′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 3′ exon or a contiguous fragment of about 1-about 50 nucleotides starting from the 5′ terminal nucleotide of the native 3′ exon, and
the 5′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 5′ exon or a contiguous fragment of about 1-about 50 nucleotides starting from the 3′ terminal nucleotide of the native 5′ exon.
119. The circular RNA precursor of any one of items 77-118, wherein the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of GAGGUCUU, AGAGACUU, UCCAGCUU or UCUGGCAU.
120. The circular RNA precursor of any one of items 77-119, wherein the ribozyme recognition site I comprises or consists of from 5′ to 3′ direction a nucleotide motif consisted of any of SEQ ID NO: 95-116, in particular the nucleotide motif consists of CUU, CUC or CAU.
121. The circular RNA precursor of any one of items 77-120, wherein the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AAACCUC, AAUCUCU, AACUGGA or AACCAGC.
122. The circular RNA precursor of any one of items 77-121, wherein the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.
123. The circular RNA precursor of any one of items 77-122, wherein,
1) the ribozyme recognition site I comprises or consists of a nucleotide sequence of SEQ ID NO: 58 and the ribozyme recognition site II comprises or consists of a nucleotide sequence of SEQ ID NO: 57;
2) the ribozyme recognition site I comprises or consists of a nucleotide sequence of SEQ ID NO: 60 and the ribozyme recognition site II comprises or consists of a nucleotide sequence of SEQ ID NO: 59;
3) the ribozyme recognition site I comprises or consists of a nucleotide sequence of SEQ ID NO: 62 and the ribozyme recognition site II comprises or consists of a nucleotide sequence of SEQ ID NO: 61;
4) the ribozyme recognition site I comprises or consists of a nucleotide sequence of SEQ ID NO: 64 and the ribozyme recognition site II comprises or consists of a nucleotide sequence of SEQ ID NO: 63;
5) the ribozyme recognition site I comprises or consists of a nucleotide sequence of SEQ ID NO: 66 and the ribozyme recognition site II comprises or consists of a nucleotide sequence of SEQ ID NO: 65;
6) the ribozyme recognition site I comprises or consists of a nucleotide sequence of SEQ ID NO: 68 and the ribozyme recognition site II comprises or consists of a nucleotide sequence of SEQ ID NO: 67; or
7) the ribozyme recognition site I comprises or consists of a nucleotide sequence of SEQ ID NO: 70 and the ribozyme recognition site II comprises or consists of a nucleotide sequence of SEQ ID NO: 69;
preferably, the ribozyme recognition site I is selected from a nucleotide sequence of UGUAACUU, and the ribozyme recognition site II is selected from a nucleotide sequence of AGAAGUAAC;
the ribozyme recognition site I is selected from a nucleotide sequence of UGUAACUU, and the ribozyme recognition site II is selected from a nucleotide sequence of AAAAUUACA;
the ribozyme recognition site I is selected from a nucleotide sequence of GAGGUCUU, and the ribozyme recognition site II is selected from a nucleotide sequence of AAACCUC;
the ribozyme recognition site I is selected from a nucleotide sequence of AGAGACUU, and the ribozyme recognition site II is selected from a nucleotide sequence of AAUCUCU;
the ribozyme recognition site I is selected from a nucleotide sequence of UCCAGCUU, and the ribozyme recognition site II is selected from a nucleotide sequence of AACUGGA; or
the ribozyme recognition site I is selected from a nucleotide sequence of UCUGGCAU, and the ribozyme recognition site II is selected from a nucleotide sequence of AACCAGC.
124. The circular RNA precursor of any one of items 77-123, wherein the circular RNA precursor further comprises a homology arm sequence I and a homology arm sequence II capable of complementary pairing to each other to form a homology arm double-stranded region.
125. The circular RNA precursor of item 124, wherein the homology arm sequence I is upstream of the 3′ self-splicing intron fragment and the homology arm sequence II is downstream of the 5′ self-splicing intron fragment.
126. The circular RNA precursor of any one of items 124-125, wherein the homology arm sequence I or the homology arm sequence II is about 5-500, about 5-450, about 5-400, about 5-350, about 5-300, about 5-250, about 5-200, about 5-150, about 5-100, about 5-80, about 5-60, about 5-50, about 5-40, about 5-30, about 5-25, about 5-20, about 5-15 or about 5-10 nucleotides in length, preferably, about 20, 25, 40, 50, 100, or 300 nucleotides in length.
127. The circular RNA precursor of any one of items 124-126, wherein the two homology arm sequences may be polyA and polyU, respectively, or polyG and polyC, respectively.
128. The circular RNA precursor of any one of items 124-127, wherein one of the homology arm sequence I and the homology arm sequence II comprises a nucleotide sequence of any one of SEQ ID NO: 71-94 or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94, while the other homology arm sequence comprises the corresponding complementary sequence.
129. The circular RNA precursor of any one of items 77-128, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is selected from CVB3, HRV-B3, BRAV-1_L, PV1_L, CAV2_L, BRAV-1, PV1, or CAV2.
130. The circular RNA precursor of any one of items 77-129, wherein the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 120-526.
131. The circular RNA precursor of any one of items 77-130, wherein the at least one protein-coding sequence encodes a protein of eukaryotic, prokaryotic or viral origin, for example, a protein for therapeutic or diagnostic use.
132. The circular RNA precursor of any one of items 77-131, wherein the at least one protein-coding sequence encodes IL-2, POLR2A, or an aquaporin.
133. The circular RNA precursor of item 132, wherein the aquaporin is AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
134. The circular RNA precursor of any one of items 77-133, wherein the at least one protein-coding sequence comprises a nucleotide sequence of any one of SEQ ID NO: 117-118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.
135. The circular RNA precursor of any one of items 77-134, which comprises a nucleotide sequence selected from any one of SEQ ID NO: 530, 531 and 533-534, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 530, 531 and 533-534.
136. A circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a translation initiation element fragment II, at least one protein-coding sequence, and a translation initiation element fragment I;
wherein the translation initiation element fragment I is derived from a 5′ terminal portion of a translation initiation element, and the translation initiation element fragment II is derived from a 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation;
optionally, the at least one protein-coding sequence comprises at least two protein-coding sequences, and a further translation initiation element, 2A sequence or spacer may be linked between any two adjacent protein-coding sequences, and the translation initiation element comprises: IRES sequence, 5′ UTR sequence, Kozak sequence, a sequence containing m6A, a sequence complementary to ribosome 18S rRNA, or any combination thereof.
137. The circular RNA precursor of item 136, wherein the self-splicing intron is selected from Group I introns and Group II introns, for example, the self-splicing intron is selected from Group I introns.
138. The circular RNA precursor of item 136 or 137, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA Met group I intron (Sh), or Agrobacterium-tumefaciens group I intron (At), for example, the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
139. The circular RNA precursor of any one of items 136-138, wherein the 3′ self-splicing intron fragment is derived from a 3′ terminal portion of a self-splicing intron starting from an intron internal split site I to the 3′ end of the self-splicing intron, and the 5′ self-splicing intron fragment is derived from a 5′ terminal portion of the self-splicing intron starting from the 5′ end of the self-splicing intron to an intron internal split site II.
140. The circular RNA precursor of any one of items 136-139, wherein when the self-splicing intron is cyanobacterium Anabaena Group I Intron such as AnaX, the intron internal split site II and the intron internal split site I are a nucleotide A at position 115 and a nucleotide A at position 116 from 5′ end of the AnaX Intron (SEQ ID NO:1) respectively, and when connected form a nucleotide sequence of AA.
141. The circular RNA precursor of any one of items 136-139, wherein when the self-splicing intron is Azoarcus Group I Intron (Azo) (SEQ ID NO:2),
1) the intron internal split site II and the intron internal split site I are a nucleotide A at position 24 and a nucleotide A at position 25 from 5′ end of the Azo intron respectively, and when connected form a nucleotide sequence of AA (P2);
2) the intron internal split site II and the intron internal split site I are a nucleotide A at position 85 and a nucleotide A at position 86 from 5′ end of the Azo intron respectively, and when connected form a nucleotide sequence of AA (P4-5);
3) the intron internal split site II and the intron internal split site I are a nucleotide C at position 71 and a nucleotide C at position 72 from 5′ end of the Azo intron respectively, and when connected form a nucleotide sequence of CC (P5);
4) the intron internal split site II and the intron internal split site I are a nucleotide C at position 108 and a nucleotide U at position 109 from 5′ end of the Azo intron respectively, and when connected form a nucleotide sequence of CU (P6);
5) the intron internal split site II and the intron internal split site I are a nucleotide A at position 154 and a nucleotide A at position 155 from 5′ end of the Azo intron respectively, and when connected form a nucleotide sequence of AA (P8); or
6) the intron internal split site II and the intron internal split site I are a nucleotide C at position 198 and a nucleotide A at position 199 from 5′ end of the Azo intron respectively, and when connected form a nucleotide sequence of CA (P9).
142. The circular RNA precursor of any one of items 136-139, wherein when the self-splicing intron is Scytalidium dimidiatum Group I Intron (Sd) (SEQ ID NO:4),
1) the intron internal split site II and the intron internal split site I are a nucleotide A at position 88 and a nucleotide G at position 89 from 5′ end of the Sd intron respectively, and when connected form a nucleotide sequence of AG (P2);
2) the intron internal split site II and the intron internal split site I are a nucleotide U at position 173 and a nucleotide G at position 174 from 5′ end of the Sd intron respectively, and when connected form a nucleotide sequence of UG (P5);
3) the intron internal split site II and the intron internal split site I are a nucleotide A at position 204 and a nucleotide G at position 205 from 5′ end of the Sd intron respectively, and when connected form a nucleotide sequence of AG (P6);
4) the intron internal split site II and the intron internal split site I are a nucleotide C at position 252 and a nucleotide G at position 253 from 5′ end of the Sd intron respectively, and when connected form a nucleotide sequence of CG (P8); or
5) the intron internal split site II and the intron internal split site I are a nucleotide C at position 370 and a nucleotide G at position 371 from 5′ end of the Sd intron respectively, and when connected form a nucleotide sequence of CG (P9).
143. The circular RNA precursor of any one of items 136-139, wherein when the self-splicing intron is Staphylococcus phage Twort Group I Intron (Twort) (SEQ ID NO:3),
1) the intron internal split site II and the intron internal split site I are a nucleotide U at position 70 and a nucleotide A at position 71 from 5′ end of the Twort intron respectively, and when connected form a nucleotide sequence of UA (P5);
2) the intron internal split site II and the intron internal split site I are a nucleotide U at position 103 and a nucleotide A at position 104 from 5′ end of the Twort intron respectively, and when connected form a nucleotide sequence of UA (P6);
3) the intron internal split site II and the intron internal split site I are a nucleotide A at position 132 and a nucleotide U at position 133 from 5′ end of the Twort intron respectively, and when connected form a nucleotide sequence of AU (P7);
4) the intron internal split site II and the intron internal split site I are a nucleotide U at position 173 and a nucleotide C at position 174 from 5′ end of the Twort intron respectively, and when connected form a nucleotide sequence of UC (P8);
5) the intron internal split site II and the intron internal split site I are a nucleotide A at position 206 and a nucleotide A at position 207 from 5′ end of the Twort intron respectively, and when connected form a nucleotide sequence of AA (P9);
6) the intron internal split site II and the intron internal split site I are a nucleotide G at position 226 and a nucleotide U at position 227 from 5′ end of the Twort intron respectively, and when connected form a nucleotide sequence of GU (P9.1);
7) the intron internal split site II and the intron internal split site I are a nucleotide U at position 23 and a nucleotide A at position 24 from 5′ end of the Twort intron respectively, and when connected form a nucleotide sequence of UA (P2); or
8) the intron internal split site II and the intron internal split site I are a nucleotide A at position 82 and a nucleotide A at position 83 from 5′ end of the Twort intron respectively, and when connected form a nucleotide sequence of AA (P4-5).
144. The circular RNA precursor of any one of items 136-139, wherein when the self-splicing intron is Scytonema-hofmani tRNA fMet group I intron (Sh) (SEQ ID NO:5),
1) the intron internal split site II and the intron internal split site I are a nucleotide A at position 129 and a nucleotide A at position 130 from 5′ end of the Sh intron respectively, and when connected form a nucleotide sequence of AA (P6);
2) the intron internal split site II and the intron internal split site I are a nucleotide A at position 60 and a nucleotide A at position 61 from 5′ end of the Sh intron respectively, and when connected form a nucleotide sequence of AA (P2);
3) the intron internal split site II and the intron internal split site I are a nucleotide A at position 109 and a nucleotide A at position 110 from 5′ end of the Sh intron respectively, and when connected form a nucleotide sequence of AA (P4-5); or
4) the intron internal split site II and the intron internal split site I are a nucleotide A at position 213 and a nucleotide A at position 214 from 5′ end of the Sh intron respectively, and when connected form a nucleotide sequence of UA (P9).
145. The circular RNA precursor of any one of items 136-139, wherein when the self-splicing intron is Agrobacterium-tumefaciens group I intron (At) (SEQ ID NO:6), the intron internal split site II and the intron internal split site I are a nucleotide A at position 107 and a nucleotide A at position 108 from 5′ end of the At intron respectively, and when connected form a nucleotide sequence of AA (P6).
146. The circular RNA precursor of any one of items 136-145, wherein the 3′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 3′ terminal portion of the self-splicing intron, and the 5′ self-splicing intron fragment is a sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the 5′ terminal portion of the self-splicing intron.
147. The circular RNA precursor of any one of items 136-146, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
148. The circular RNA precursor of any one of items 136-146, wherein the self-splicing intron is the Azoarcus Group I Intron (Azo), the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.
149. The circular RNA precursor of any one of items 136-146, wherein the self-splicing intron is the Scytalidium dimidiatum Group I Intron (Sd), the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.
150. The circular RNA precursor of any one of items 136-146, wherein the self-splicing intron is the Staphylococcus phage Twort Group I Intron (Twort), the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.
151. The circular RNA precursor of any one of items 136-146, wherein the self-splicing intron is the Scytonema-hofmani tRNA fMet group I intron (Sh), the 3′ self-splicing intron fragment comprises or consists of a nucleotide sequence of SEQ ID NO: 47, 49, 51 or 53 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51 or 53, and the 5′ self-splicing intron fragment comprises or consists of a nucleotide sequence of SEQ ID NO: 48, 50, 52 or 54 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52 or 54.
152. The circular RNA precursor of any one of items 136-146, wherein the self-splicing intron is the Agrobacterium-tumefaciens group I intron (At), the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.
153. The circular RNA precursor of any one of items 136-152, wherein the ribozyme recognition site I and the ribozyme recognition site II are configured such that the circular RNA comprising them has reduced immunogenicity.
154. The circular RNA precursor of any one of items 136-153, wherein the ribozyme recognition site I and the ribozyme recognition site II are configured such that the circular RNA precursor comprising them has comparable or increased circularization efficiency.
155. The circular RNA precursor of any one of items 136-154, wherein the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.
156. The circular RNA precursor of any one of items 136-155, wherein the total length of the ribozyme recognition site I is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.
157. The circular RNA precursor of any one of items 136-156, wherein the ribozyme recognition site I and the ribozyme recognition site II are configured to be capable of forming a stem-loop structure, e.g., upon self-splicing for circularization.
158. The circular RNA precursor of item 157, wherein the loop of the stem-loop structure comprises the splicing junction.
159. The circular RNA precursor of any one of items 157-158, wherein the ribozyme recognition site I comprises the sequence structure of the following formula: 5′-third non-pairing sequence-third pairing sequence-third loop sequence-3′; and the ribozyme recognition site II comprises the sequence structure of the following formula: 5′-fourth loop sequence-fourth pairing sequence-fourth non-pairing sequence-3′,
wherein the third non-pairing sequence or the fourth non-pairing sequence may be independently present or absent,
the third pairing sequence and the fourth pairing sequence can complementarily pair to each other to form the stem of the stem-loop structure, wherein the third loop sequence and the fourth loop sequence can form the loop of the stem-loop structure, e.g., through self-splicing for circularization.
160. The circular RNA precursor of any one of items 136-159, wherein the ribozyme recognition site II can be selected from a various sequence.
161. The circular RNA precursor of item 159 or 160, wherein the fourth loop sequence comprises or consists of one or more nucleotides which can pair with the P1 region of the corresponding self-splicing intron to form a P10 duplex region during the circularization.
162. The circular RNA precursor of any one of items 159-161, wherein the fourth loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.
163. The circular RNA precursor of any one of items 159-162, wherein the fourth loop sequence comprises or consists of the sequence of AAAA, AGAA, AA, UUUU, UAAA, CAAA, or GAAA, preferably AAAA.
164. The circular RNA precursor of any one of items 136-163, wherein the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.
165. The circular RNA precursor of any one of items 136-164, wherein the third loop sequence comprises or consists of one or more nucleotides which can pair with the internal guide sequence (IGS) of the corresponding self-splicing intron to form a P1 duplex region during the circularization.
166. The circular RNA precursor of any one of items 159-165, wherein the third loop sequence comprises or consists of a nucleotide sequence of any one of SEQ ID NO: 95-116, such as CUU, CAU or CUC, preferably CUU.
167. The circular RNA precursor of any one of items 159-166, wherein the loop of the stem-loop structure has a sequence of CUUAAAA, CUUUUUU, CUUAA, CUUGAAA, CUUUAAA, CUUAGAA, CUUCAAA or CUCAAAA, preferably CUUAAAA.
168. The circular RNA precursor of any one of items 159-167, wherein the stem portion of the stem-loop structure comprises 2-15 or more consecutive matched base pairs, preferably, the stem portion of the stem-loop structure comprises 5, 6 or 7 consecutive matched base pairs.
169. The circular RNA precursor of any one of items 159-168, wherein the stem portion in the stem-loop structure comprises up to 2 base pair mismatches, or the stem portion comprises only 1 base pair mismatch, preferably, the stem portion comprises no base pair mismatches.
170. The circular RNA precursor of any one of items 159-169, wherein the third pairing sequence comprises only G and the fourth pairing sequence comprises only C, or the third pairing sequence comprises only C and the fourth pairing sequence comprises only G.
171. The circular RNA precursor of any one of items 159-169, wherein the third pairing sequence comprises only A and the fourth pairing sequence comprises only U, or the third pairing sequence comprises only U and the fourth pairing sequence comprises only A.
172. The circular RNA precursor of any one of items 159-171, wherein the ribozyme recognition site II comprises or consists of a 3′ exon region, and the ribozyme recognition site I comprises or consists of a 5′ exon region.
173. The circular RNA precursor of item 172, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.
174. The circular RNA precursor of item 172 or 173, wherein the 3′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 3′ exon or a contiguous fragment of about 1-about 50 nucleotides starting from the 5′ terminal nucleotide of the native 3′ exon, and
the 5′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the native 5′ exon or a contiguous fragment of about 1-about 50 nucleotides starting from the 3′ terminal nucleotide of the native 5′ exon.
175. The circular RNA precursor of any one of items 136-174, wherein the ribozyme recognition site I comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 58, 60, 62, 64, 66, 68 or 70; preferably, the ribozyme recognition site I comprises a nucleotide sequence of UGUAACUU.
176. The circular RNA precursor of any one of items 136-175, wherein the ribozyme recognition site I comprises or consists of from 5′ to 3′ direction a nucleotide motif consisted of any of SEQ ID NO: 95-116, in particular the nucleotide motif consists of CUU, CUC or CAU.
177. The circular RNA precursor of any one of items 136-176, wherein the ribozyme recognition site II comprises or consists of a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence selected from SEQ ID NO: 57, 59, 61, 63, 65, 67 or 69; preferably, the ribozyme recognition site II comprises a nucleotide sequence of AGAAGUAAC or AAAAUUACA.
178. The circular RNA precursor of any one of items 136-177, wherein the ribozyme recognition site I comprises or consists of a nucleotide sequence of any one of SEQ ID NO: 58, 60, 62, 64, 66, 68 or 70 and the ribozyme recognition site II comprises or consists of a nucleotide sequence of any one of SEQ ID NO: 57, 59, 61, 63, 65, 67 or 69.
179. The circular RNA precursor of any one of items 136-178, wherein,
1) the ribozyme recognition site I comprises or consists of a nucleotide sequence of SEQ ID NO: 58 and the ribozyme recognition site II comprises or consists of a nucleotide sequence of SEQ ID NO: 57;
2) the ribozyme recognition site I comprises or consists of a nucleotide sequence of SEQ ID NO: 60 and the ribozyme recognition site II comprises or consists of a nucleotide sequence of SEQ ID NO: 59;
3) the ribozyme recognition site I comprises or consists of a nucleotide sequence of SEQ ID NO: 62 and the ribozyme recognition site II comprises or consists of a nucleotide sequence of SEQ ID NO: 61;
4) the ribozyme recognition site I comprises or consists of a nucleotide sequence of SEQ ID NO: 64 and the ribozyme recognition site II comprises or consists of a nucleotide sequence of SEQ ID NO: 63;
5) the ribozyme recognition site I comprises or consists of a nucleotide sequence of SEQ ID NO: 66 and the ribozyme recognition site II comprises or consists of a nucleotide sequence of SEQ ID NO: 65;
6) the ribozyme recognition site I comprises or consists of a nucleotide sequence of SEQ ID NO: 68 and the ribozyme recognition site II comprises or consists of a nucleotide sequence of SEQ ID NO: 67; or
7) the ribozyme recognition site I comprises or consists of a nucleotide sequence of SEQ ID NO: 70 and the ribozyme recognition site II comprises or consists of a nucleotide sequence of SEQ ID NO: 69;
preferably, the ribozyme recognition site I is selected from a nucleotide sequence of UGUAACUU, and the ribozyme recognition site II is selected from a nucleotide sequence of AGAAGUAAC;
the ribozyme recognition site I is selected from a nucleotide sequence of UGUAACUU, and the ribozyme recognition site II is selected from a nucleotide sequence of AAAAUUACA;
the ribozyme recognition site I is selected from a nucleotide sequence of GAGGUCUU, and the ribozyme recognition site II is selected from a nucleotide sequence of AAACCUC;
the ribozyme recognition site I is selected from a nucleotide sequence of AGAGACUU, and the ribozyme recognition site II is selected from a nucleotide sequence of AAUCUCU;
the ribozyme recognition site I is selected from a nucleotide sequence of UCCAGCUU, and the ribozyme recognition site II is selected from a nucleotide sequence of AACUGGA; or
the ribozyme recognition site I is selected from a nucleotide sequence of UCUGGCAU, and the ribozyme recognition site II is selected from a nucleotide sequence of AACCAGC.
180. The circular RNA precursor of any one of items 136-179, wherein the circular RNA precursor further comprises a homology arm sequence I and a homology arm sequence II capable of complementary pairing to each other to form a homology arm double-stranded region.
181. The circular RNA precursor of item 180, wherein the homology arm sequence I is upstream of the 3′ self-splicing intron fragment and the homology arm sequence II is downstream of the 5′ self-splicing intron fragment.
182. The circular RNA precursor of any one of items 180-181, wherein the homology arm sequence I or the homology arm sequence II is about 5-500, about 5-450, about 5-400, about 5-350, about 5-300, about 5-250, about 5-200, about 5-150, about 5-100, about 5-80, about 5-60, about 5-50, about 5-40, about 5-30, about 5-25, about 5-20, about 5-15 or about 5-10 nucleotides in length, preferably, about 20, 25, 40, 50, 100, or 300 nucleotides in length.
183. The circular RNA precursor of any one of items 180-182, wherein the two homology arm sequences may be polyA and polyU, respectively, or polyG and polyC, respectively.
184. The circular RNA precursor of any one of items 180-183, wherein one of the homology arm sequence I and the homology arm sequence II has the nucleotide sequence of any one of SEQ ID NO: 71-94 or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94, while the other homology arm sequence has the corresponding complementary sequence.
185. The circular RNA precursor of any one of items 136-184, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is selected from CVB3, HRV-B3, BRAV-1_L, PV1_L, CAV2_L, BRAV-1, PV1, or CAV2.
186. The circular RNA precursor of any one of items 136-185, wherein the IRES comprises a nucleotide sequence set forth in one of SEQ ID NOs: 120-526, or comprise a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 120-526.
187. The circular RNA precursor of any one of items 136-186, wherein the at least one protein-coding sequence encodes a protein of eukaryotic, prokaryotic or viral origin, for example, a protein for therapeutic or diagnostic use.
188. The circular RNA precursor of any one of items 136-187, wherein the at least one protein-coding sequence encodes IL-2, POLR2A, or an aquaporin, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
189. The circular RNA precursor of any one of items 136-188, wherein the at least one protein-coding sequence comprises a nucleotide sequence of any one of SEQ ID NO: 117-118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.
190. The circular RNA precursor of any one of items 136-189, which comprises a nucleotide sequence selected from any one of SEQ ID NO: 528, 529 and 535-536, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 528, 529 and 535-536.
191. A circular RNA precursor comprising from 5′ to 3′ direction:
a) a 3′ self-splicing intron fragment;
b) a nucleotide sequence of interest;
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-coding sequence fragment II, and a non-coding sequence fragment I;
wherein the non-coding sequence fragment I is derived from a 5′ terminal portion of a non-coding sequence, and the non-coding sequence fragment II is derived from a 3′ terminal portion of the non-coding sequence, and the non-coding sequence fragment I comprises a ribozyme recognition site I located at its 3′ end, the non-coding sequence fragment II comprises a ribozyme recognition site II located at its 5′ end, wherein in the generated circular RNA the ribozyme recognition site I is connected with the ribozyme recognition site II, and thus the non-coding sequence fragment I is connected with the non-coding sequence fragment II to form the complete non-coding sequence.
192. The circular RNA precursor of item 191, wherein the non-coding sequence is an endogenous circRNA.
193. A circular RNA precursor comprising from 5′ to 3′ direction:
a) a 3′ self-splicing intron fragment;
b) a nucleotide sequence of interest; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the nucleotide sequence of interest through the self-splicing of the circular RNA precursor;
wherein the nucleotide sequence of interest comprises from 5′ to 3′ direction: a non-TIE functional element fragment II, a translation initiation element, and at least one protein-coding sequence, and a non-TIE functional element fragment I;
wherein the non-TIE functional element fragment I is derived from a 5′ terminal portion of a non-TIE functional element, and the non-TIE translation initiation element fragment II is derived from a 3′ terminal portion of the non-TIE functional element, and
the non-TIE functional element fragment I comprises a ribozyme recognition site I located at its 3′ end, the non-TIE functional fragment II comprises a ribozyme recognition site II located at its 5′ end, wherein in the generated circular RNA the ribozyme recognition site I is connected with the ribozyme recognition site II, and thus the non-TIE functional element fragment I is connected with the non-TIE functional element fragment II to form the non-TIE functional element.
194. The circular RNA precursor of item 193, wherein the non-TIE functional element is selected from 3′ UTR or a replicon.
195. The circular RNA precursor of any one of items 191-194, wherein the self-splicing intron, the 3′ self-splicing intron fragment, or the 5′ self-splicing intron fragment is defined as in any one of items 136-190.
196. The circular RNA precursor of any one of items 191-195, wherein the ribozyme recognition site I is as defined in any one of items 136-190 and/or the ribozyme recognition site II is as defined in any one of items 136-190.
197. The circular RNA precursor of any one of items 191-196, wherein the circular RNA precursor further comprises a homology arm sequence I and a homology arm sequence II, which are as defined in any one of items 180-184.
198. The circular RNA precursor of any one of items 191-197, wherein the translation initiation element is as defined in any one of items 185-186.
199. The circular RNA precursor of any one of items 193-198, wherein the at least one protein-coding sequence is as defined in any one of items 136-190.
200. A circular RNA precursor comprising from 5′ to 3′ direction:
a) a 3′ self-splicing intron fragment;
b) a nucleotide sequence of interest, comprising from 5′ to 3′ direction: protein-coding sequence fragment II, a translation initiation element such as an internal ribosome entry site (IRES) and a protein-coding sequence fragment I; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising the protein-coding sequence fragment II, the translation initiation element, and the protein-coding sequence fragment I through the self-splicing of the circular RNA precursor;
wherein the protein-coding sequence fragment I is derived from a 5′ terminal portion of at least one protein-coding sequence, and the protein-coding sequence fragment II is derived from a 3′ terminal portion of at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence;
wherein the at least one protein-coding sequence encodes an aquaporin, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
201. The circular RNA precursor of item 200, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At).
202. The circular RNA precursor of item 200 or 201, wherein the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
203. The circular RNA precursor of any one of items 200-202, wherein the self-splicing intron is selected from a nucleotide sequence of any one of SEQ ID NO: 1 and 3, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1 and 3.
204. The circular RNA precursor of any one of items 200-203, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
205. The circular RNA precursor of any one of items 200-203, wherein the self-splicing intron is the Staphylococcus phage Twort Group I Intron (Twort), the 3′ self-splicing intron fragment comprises or consists of a nucleotide sequence of SEQ ID NO: 27 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 27, and the 5′ self-splicing intron fragment comprises or consists of a nucleotide sequence of SEQ ID NO: 28 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 28.
206. The circular RNA precursor of any one of items 200-205, wherein the total length of the ribozyme recognition site I and the ribozyme recognition site II is about 0 to about 35 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length.
207. The circular RNA precursor of any one of items 200-206, wherein the total length of the ribozyme recognition site I is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length.
208. The circular RNA precursor of any one of items 200-207, wherein the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 58, 60 or 64.
209. The circular RNA precursor of any one of items 200-208, wherein the ribozyme recognition site I comprises or consists of from 5′ to 3′ direction a nucleotide motif consisted of any of SEQ ID NO: 95-116, in particular the nucleotide motif consists of CUU, CUC or CAU.
210. The circular RNA precursor of any one of items 200-209, wherein the ribozyme recognition site I comprises a nucleotide sequence of UCCAGCUU or UCUGGCAU.
211. The circular RNA precursor of any one of items 200-210, wherein the ribozyme recognition site II comprises a nucleotide sequence of AACUGGA or AACCAGC.
212. The circular RNA precursor of any one of items 200-211, wherein the circular RNA precursor further comprises a homology arm sequence I and a homology arm sequence II capable of complementary pairing to each other to form a homology arm double-stranded region.
213. The circular RNA precursor of item 212, wherein the homology arm sequence I is upstream of the 3′ self-splicing intron fragment and the homology arm sequence II is downstream of the 5′ self-splicing intron fragment.
214. The circular RNA precursor of any one of items 212-213, wherein the homology arm sequence I or the homology arm sequence II is about 5-50 nucleotides in length, preferably, about 10, 20, 30 or 40 nucleotides in length.
215. The circular RNA precursor of any one of items 212-214, wherein one of the homology arm sequence I and the homology arm sequence II comprises a nucleotide sequence of any one of SEQ ID NO: 71-94 such as 83-84, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94 such as 83-84, while the other homology arm sequence comprises the corresponding complementary sequence.
216. The circular RNA precursor of any one of items 200-215, wherein the translation initiation element is an internal ribosome entry site (IRES) which is selected from CVB3, HRV-B3, BRAV-1_L, PV1_L, CAV2_L, BRAV-1, PV1, or CAV2, preferably CVB3.
217. The circular RNA precursor of any one of items 200-216, wherein the IRES comprises a nucleotide sequence of SEQ ID NO: 121, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 121.
218. The circular RNA precursor of any one of items 200-217, wherein the at least one protein-coding sequence comprises an AQP1-coding sequence, such as a nucleotide sequence of SEQ ID NO: 118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 118.
219. The circular RNA precursor of any one of items 200-218, which comprises a nucleotide sequence selected from any one of SEQ ID NO: 533-534, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 533-534.
220. A circular RNA precursor comprising from 5′ to 3′ direction:
a) a 3′ self-splicing intron fragment;
b) a nucleotide sequence of interest, comprising from 5′ to 3′ direction: a translation initiation element fragment II, at least one protein-coding sequence encoding an aquaporin and a translation initiation element fragment I; and
c) a 5′ self-splicing intron fragment;
wherein the 3′ self-splicing intron fragment and the 5′ self-splicing intron fragment in combination retain the self-splicing activity of a self-splicing intron and allow generation of a circular RNA comprising a translation initiation element fragment II, at least one protein-coding sequence, and a translation initiation element fragment I through the self-splicing of the circular RNA precursor;
wherein the translation initiation element fragment I is derived from a 5′ terminal portion of a translation initiation element, and the translation initiation element fragment II is derived from a 3′ terminal portion of the translation initiation element, and
the translation initiation element fragment I comprises a ribozyme recognition site I located at its 3′ end, the translation initiation element fragment II comprises a ribozyme recognition site II located at its 5′ end, wherein in the generated circular RNA the ribozyme recognition site I is connected with the ribozyme recognition site II, and thus the translation initiation element fragment I is connected with the translation initiation element fragment II to form the translation initiation element which can recruit ribosome to initiate translation;
wherein the aquaporin is preferably AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
221. The circular RNA precursor of item 220, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At).
222. The circular RNA precursor of item 220 or 221, wherein the self-splicing intron is selected from a nucleotide sequence of any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.
223. The circular RNA precursor of any one of items 220-222, wherein the self-splicing intron is selected from a nucleotide sequence of any one of SEQ ID NO: 1, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1.
224. The circular RNA precursor of any one of items 220-223, wherein the self-splicing intron is the cyanobacterium Anabaena Group I Intron such as AnaX, the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.
225. The circular RNA precursor of any one of items 220-224, wherein the total length of the ribozyme recognition site I and the ribozyme recognition site II is about 0 to about 35 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length.
226. The circular RNA precursor of any one of items 220-225, wherein the total length of the ribozyme recognition site I is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length.
227. The circular RNA precursor of any one of items 220-226, wherein the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 58 or 60.
228. The circular RNA precursor of any one of items 220-227, wherein the ribozyme recognition site I comprises or consists of from 5′ to 3′ direction a nucleotide motif consisted of any of SEQ ID NO: 95-116, in particular the nucleotide motif consists of CUU, CUC or CAU.
229. The circular RNA precursor of any one of items 220-228, wherein the ribozyme recognition site I comprises a nucleotide sequence of UGUAACUU.
230. The circular RNA precursor of any one of items 220-229, wherein the ribozyme recognition site II comprises a nucleotide sequence of AGAAGUAAC or AAAAUUACA.
231. The circular RNA precursor of any one of items 220-230, wherein the circular RNA precursor further comprises a homology arm sequence I and a homology arm sequence II capable of complementary pairing to each other to form a homology arm double-stranded region.
232. The circular RNA precursor of item 231, wherein the homology arm sequence I is upstream of the 3′ self-splicing intron fragment and the homology arm sequence II is downstream of the 5′ self-splicing intron fragment.
233. The circular RNA precursor of any one of items 231-232, wherein the homology arm sequence I or the homology arm sequence II is about 5-500 nucleotides in length, preferably, about 10, 20, 30, 40 or 50 nucleotides in length.
234. The circular RNA precursor of any one of items 231-233, wherein one of the homology arm sequence I and the homology arm sequence II comprises a nucleotide sequence of any one of SEQ ID NO: 71-94 such as 83-84, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 71-94 such as 83-84, while the other homology arm sequence comprises the corresponding complementary sequence.
235. The circular RNA precursor of any one of items 220-234, wherein the translation initiation element is an internal ribosome entry site (IRES) which is selected from CVB3, HRV-B3, BRAV-1_L, PV1_L, CAV2_L, BRAV-1, PV1, or CAV2, preferably CVB3.
236. The circular RNA precursor of any one of items 220-235, wherein the TRES comprises a nucleotide sequence of SEQ ID NO: 121, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 121.
237. The circular RNA precursor of any one of items 220-236, wherein the at least one protein-coding sequence comprises a AQP-1-coding sequence, such as a nucleotide sequence of SEQ ID NO: 118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 118.
238. The circular RNA precursor of any one of items 220-237, which comprises a nucleotide sequence selected from any one of SEQ ID NO: 535-536, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 535-536.
239. A nucleic acid vector for generating a circular RNA molecule, said vector comprises a coding sequence of the circular RNA precursor of any one of items 1-238.
240. The nucleic acid vector of item 239, which further comprises an RNA polymerase promoter sequence operably linked to the coding sequence of the circular RNA precursor.
241. The nucleic acid vector of item 240, wherein the promoter is a T7 RNA polymerase promoter, a T6 viral RNA polymerase promoter, a SP6 viral RNA polymerase promoter, a T3 viral RNA polymerase promoter or a T4 viral RNA polymerase promoter, preferably a T7 RNA polymerase promoter.
242. A method for preparing a circular RNA, comprising:
1) providing the circular RNA precursor of any one of items 1-238 or obtaining a circular RNA precursor by transcribing from the nucleic acid vector of any one of items 239-241;
2) incubating the circular RNA precursor in the presence of a divalent metal cation at a temperature at which RNA circularization occurs; and
3) harvesting the circular RNA obtained in step 2).
243. The method of item 242, wherein the divalent metal cation is Mg²⁺ and/or Mn²⁺.
244. The method of item 242 or 243, wherein the concentration of the divalent metal cation is about 5 mM to about 550 mM.
245. A method for preparing a circular RNA, comprising
a) providing a nucleic acid vector comprising a self-splicing intron-based RNA circularizing elements as a transcription template; and
b) incubating the nucleic acid vector in an in vitro transcription system comprising a divalent metal cation and an RNA polymerase for a first time period during which the linear RNA produced by in vitro transcription is self-circularized under the action of the RNA circularizing elements to produce a circular RNA.
246. The method of item 245, wherein the nucleic acid vector is the nucleic acid vector of any one of items 239-241.
247. The method of item 245 or 246, wherein the divalent metal cation in the in vitro transcription system is Mg²⁺.
248. The method of any one of items 245-247, wherein the in vitro transcription system further comprises a monovalent metal cation and/or a monovalent anion.
249. The method of item 248, wherein the monovalent metal cation is Na⁺ or K⁺.
250. The method of item 248, wherein the monovalent anion is Cl⁻ or CH₃COO⁻ (OAc).
251. The method of any one of items 245-250, wherein the concentration of the divalent metal cation in the system during the first time period is from about 5 mM to about 50 mM.
252. The method of any one of items 248-251, wherein the concentration of the monovalent metal cation in the system during the first time period is from about 5 mM to about 100 mM.
253. The method of any one of items 248-252, wherein the concentration of the monovalent anion in the system during the first time period is from about 5 mM to about 150 mM.
254. The method of any one of items 245-253, wherein the method does not comprise a step of isolating and/or purifying the linear RNA produced by the in vitro transcription.
255. The method of any one of items 245-254, wherein the buffer of the in vitro transcription system is selected from Tris-HCl buffer, HEPES buffer, MES buffer, citrate buffer, or phosphate buffer, preferably, the buffer of the in vitro transcription system is HEPES buffer.
256. The method of any one of items 245-255, wherein the in vitro transcription system has a pH of about 5-about 8, preferably, the in vitro transcription system has a pH of about 7.5.
257. The method of any one of items 245-256, wherein the RNA polymerase is selected from a T7 RNA polymerase, a T6 viral RNA polymerase, a SP6 viral RNA polymerase, a T3 viral RNA polymerase, or a T4 viral RNA polymerase, preferably, the RNA polymerase is T7 RNA polymerase.
258. The method of any one of items 245-257, wherein the first time period is about 0.5 hours to about 24 hours, preferably, the first time period is 3 hours.
259. The method of any one of items 245-258, wherein the incubation for the first time period is carried out at about 16° C. to about 60° C., preferably, the incubation for the first time period is carried out at about 37° C.
260. The method of any one of items 245-259, wherein after incubation for the first time period in step b), the method further comprises step c):
adding an additional amount of metal cation to the in vitro transcription system and incubating for a second time period; or
changing the buffer of the system, adding a metal cation and incubating for a second time period.
261. The method of item 260, wherein the metal cation added for the incubation for the second time period is a divalent metal cation, such as Mg²⁺ or Mn²⁺.
262. The method of item 260 or 261, wherein during the incubation for the second time period, the metal cation is added to a final concentration of about 5 mM to about 550 mM.
263. The method of any one of items 260-262, wherein the buffer of the system is changed to Tris-HCl buffer, HEPES buffer, MES buffer, citrate buffer, or phosphate buffer during the second time period.
264. The method of any one of items 260-263, wherein the pH of the system during the second time period is 5-8.
265. The method of any one of items 260-264, wherein the second time period is about 5 minutes to about 8 hours, about 5 minutes to about 6 hours, about 5 minutes to about 4 hours, or about 5 minutes to about 2 hours.
266. The method of any one of items 260-265, wherein the incubation for the second time period is carried out at about 25° C. to about 75° C., such as about 37° C.
267. The method of any one of items 260-266, wherein the method further comprises a step of recovering or purifying the circular RNA as produced.
268. A method for purifying a circular RNA, comprising:
a) contacting a mixture comprising the circular RNA and the circular RNA precursor with a circular RNA-specific probe under a condition that allows the circular RNA-specific probe to specifically bind to and form a complex with the circular RNA;
b) separating the complex from one or more components in the mixture that are not bound to the circular RNA-specific probe; and
c) releasing the circular RNA from the complex.
269. The method of item 268, wherein the circular RNA is prepared by circularizing a circular RNA precursor.
270. The method of item 268 or 269, wherein the circular RNA is prepared by ligating both ends of a linear circular RNA precursor with an RNA ligase, or, the circular RNA is prepared by the self-splicing ribozyme activity of self-splicing intron-based circularizing elements contained in the circular RNA precursor.
271. The method of any one of items 268-270, wherein the circular RNA-specific probe is a single-stranded DNA or RNA probe.
272. The method of any one of items 268-271, wherein the circular RNA-specific probe specifically hybridizes to a region flanking the circularization junction of the circular RNA.
273. The method of any one of items 268-272, wherein the circular RNA-specific probe is about 10 nucleotides-about 35 nucleotides, about 10 nucleotides-about 50 nucleotides, about 10 nucleotides-about 80 nucleotides, or about 10 nucleotides-about 100 nucleotides in length or longer.
274. The method of any one of items 268-273, wherein the circular RNA-specific probe is immobilized on a support such as a solid support, e.g., the circular RNA-specific probe is immobilized on the support after binding to the circular RNA or the circular RNA-specific probe is pre-immobilized on the support.
275. The method of any one of items 268-274, wherein the condition in step a) include denaturing the RNA at between about 60° C. and about 95° C. for about 2 minutes to about 10 minutes, then gradually reducing the temperature to below about 40° C. to allow the circular RNA annealing to the circular RNA-specific probe.
276. The method of item 275, wherein step c) is performed by increasing the temperature to about 60° C. to about 95° C. (e.g., about 60° C., about 62° C., about 64° C., about 66° C., about 68° C., about 70° C., about 75° C., about 80° C., about 85° C., about 90° C., about 95° C.) to release the circular RNA.
277. The method of any one of items 268-276, wherein the condition in step a) comprises a high salt concentration range of 0.25M-2M.
278. The method of item 277, wherein the salt is NaCl or a guanidine salt.
279. The method of item 277 or 278, wherein in step c) the circular RNAis released by elution with an elution buffer, such as a low salt buffer.
280. The method of item 279, wherein the elution buffer is Tris-EDTA buffer (TE buffer) or water.
281. The method of any one of items 268-280, wherein in step b), the one or more components are removed by washing the complex with a washing buffer.
282. The method of any one of items 268-281, which further comprises the following steps:
i) contacting the mixture comprising the circular RNA and the circular RNA precursor to a linear circular RNA precursor-specific probe under a condition that allows the linear circular RNA precursor-specific probe to specifically bind to and form a complex with the linear circular RNA precursor;
ii) removing the complex formed by the linear circular RNA precursor-specific probe with the linear circular RNA precursor from the mixture, and
iii) collecting the circular RNA-containing mixture obtained in step ii).
283. The method of item 282, wherein steps i)-iii) are performed before step a), for example, steps i)-iii) may be performed multiple times before step a), e.g., 2, 3, 4 or more times.
284. The method of item 283, wherein steps i)-iii) are performed concurrently with steps a)-c).
285. A method for purifying a circular RNA, comprising:
i) contacting a mixture comprising the circular RNA and the circular RNA precursor to a circular RNA precursor-specific probe under a condition that allows the circular RNA precursor-specific probe to specifically bind to and form a complex with the circular RNA precursor; and
ii) removing the complex formed by the circular RNA precursor-specific probe with the circular RNA precursor from the mixture,
iii) collecting the circular RNA-containing mixture obtained in step ii), and
optionally, steps i)-iii) are performed multiple times, e.g., 2 times, 3 times, 4 times or more.
286. The method of item 285, wherein the circular RNA precursor-specific probe specifically binds to the circular RNA precursor and does not substantially bind to the circular RNA.
287. The method of item 285 or 286, wherein the circular RNA precursor-specific probe is immobilized on a support, such as a solid support, for example, the circular RNA precursor-specific probe is immobilized on the support after binding to the circular RNA precursor, or the circular RNA precursor-specific probe is pre-immobilized on the support.
288. The method of any one of items 285-287, wherein the circular RNA precursor is selected from the circular RNA precursor of any one of items 21-76,
wherein the circular RNA precursor is capable of removing the 3′ self-splicing intron fragment and the 5′ self-splicing intron fragment by self-splicing, generating a circular RNA comprising the first residual circularizing element, the nucleotide sequence of interest and the second residual circularizing element.
289. The method of item 288, wherein the circular RNA precursor-specific probe specifically hybridizes to at least a portion of the first residual circularizing element or the second residual circularizing element.
290. The method of item 288 or 289, wherein the circular RNA precursor-specific probe hybridizes to a portion of the circular RNA precursor outside the first residual circularizing element, the nucleotide sequence of interest, and the second residual circularizing element.
291. The method of any one of items 288-290, wherein the circular RNA precursor-specific probe hybridizes to the 3′ self-splicing intron fragment or a portion thereof or a 5′ flanking sequence thereof, or the 5′ self-splicing intron fragment or a portion thereof or a 3′ flanking sequence thereof.
292. The method of any one of items 288-291, wherein the circular RNA precursor contains a sequence selected from SEQ ID NOs: 600-610 or a complementary sequence thereof, preferably, SEQ ID NO:606 or a complementary sequence thereof outside the first residual circularizing element, the nucleotide sequence of interest, and the second residual circularizing element, to which the circular RNA precursor-specific probe specifically hybridizes.
293. The method of any one of items 285-287, wherein the circular RNA precursor is selected from the circular RNA precursor of any one of items 77-238,
wherein the circular RNA precursor is capable of removing the 3′ self-splicing intron fragment and the 5′ self-splicing intron fragment by self-splicing, generating a circular RNA comprising the nucleotide sequence of interest.
294. The method of item 293, wherein the circular RNA precursor-specific probe hybridizes to a portion of the circular RNA precursor outside the nucleotide sequence of interest.
295. The method of any one of items 293-294, wherein the circular RNA precursor-specific probe hybridizes to the 3′ self-splicing intron fragment or a portion thereof or a 5′ flanking sequence thereof, or the 5′ self-splicing intron fragment or a portion thereof or a 3′ flanking sequence thereof.
296. The method of any one of items 293-295, wherein the circular RNA precursor contains a sequence selected from SEQ ID NOs: 600-610 or a complementary sequence thereof, preferably, SEQ ID NO:606 or a complementary sequence thereof outside the nucleotide sequence of interest, to which the linear circular RNA precursor-specific probe specifically hybridizes.
297. The method of any one of items 285-296, wherein the molar ratio of the probe to the RNA molecules in the mixture is from about 1:1 to about 200,000:1, from about 1:1 to about 150,000:1, from about 1:1 to about 100,000:1, from about 1:1 to about 90,000:1, from about 1:1 to about 80,000:1, from about 1:1 to about 70,000:1, from about 1:1 to about 60,000:1, from about 1:1 to about 50,000:1, from about 1:1 to about 40,000:1, from about 1:1 to about 30,000:1, from about 1:1 to about 20,000:1, from about 1:1 to about 10,000:1.
298. A method for purifying a circular RNA, comprising:
i) adding a linear RNA-specific tag to a mixture comprising circular RNA and linear RNA, wherein the linear RNA-specific tag can be linked to the linear RNA;
ii) contacting the mixture comprising circular RNA and linear RNA with a linear RNA probe that specifically binds to the tag under a condition that allows the probe to specifically bind to and form a complex with the linear RNA; and
iii) removing the complex formed by the linear RNA probe with the linear RNA from the mixture,
iv) collecting the circular RNA-containing mixture obtained in step iii),
optionally, steps ii)-iv) are performed multiple times, e.g., 2 times, 3 times, 4 times or more.
299. The method of item 298, wherein the tag comprises a polyA, polyG, polyU, or polyC sequence.
300. The method of item 298 or 299, wherein the tag is about 10-200 nt in length or the probe is about 10-200 nt in length.
301. The method of any one of items 298-300, wherein the tag is added to the linear RNA by adding a PolyA/T/C/G polymerase or a ligase to the mixture.
302. The method of any one of items 298-301, wherein the linear RNA probe specifically binds to the added tag without substantially binding to the circular RNA.
303. The method of any one of items 298-302, wherein the linear RNA probe is a single-stranded DNA probe, or a single-stranded RNA probe.
304. The method of any one of items 298-303, wherein the linear RNA probe is immobilized on a support such as a solid support, for example, the linear RNA probe is immobilized on the support after binding to the linear RNA, or, the linear RNA probe is pre-immobilized on the support.
305. The method of any one of items 242-304, wherein the circular RNA comprises a nucleotide sequence of any one of SEQ ID NO: 543-558, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 543-558.
306. A circular RNA, which is prepared from the circular RNA precursor of any one of items 1-238, prepared by the method of any one of items 242-267, purified by the method of any one of items 268-305 or generated by the nucleic acid vector of any one of items 239-241.
307. The circular RNA of item 306, which comprises a nucleotide sequence of any one of SEQ ID NO: 543-558, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 543-558.
308. Use of the circular RNA precursor of any one of items 1-238 and/or the circular RNA of any one of items 306-307 as an expression vector.
309. A pharmaceutical composition comprising the circular RNA precursor of any one of items 1-238 and/or the nucleic acid vector of any one of items 239-241 and/or the circular RNA of any one of items 306-307, and a pharmaceutically acceptable carrier.
310. A product expressed by the circular RNA of any one of items 306-307.
311. The product of item 310, which comprises IL-2, POLR2A, or an aquaporin, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.
312. A method for preventing or treating a disease or condition in a subject, comprising administering to a subject the circular RNA of any one of items 306-307, or the pharmaceutical composition of item 309.
313. The method of item 312, wherein the disease or condition comprises but not limited to xerostomia, xerophthalmia, Sjogren's syndrome or the like.
314. Use of the circular RNA of any one of items 306-307 or the pharmaceutical composition of item 309 in the manufacture of a medicament for preventing or treating a disease or condition in a subject.
315. The use of item 314, wherein the disease or condition comprises but not limited to xerostomia, xerophthalmia, Sjogren's syndrome or the like.
316. A method of screening a target open reading frame (ORF) sequence encoding a target polypeptide for a nucleotide motif, comprising steps of:
1) extracting amino acid units in the direction of N terminal to C terminal in the target polypeptide containing m amino acids, and each group of amino acid units includes an amino acid with the number of n; with a step of 1 amino acid, slide along the N terminal to the C terminal, n is an integer and n≥2, m is an integer and ≥1;
2) listing all nucleotide sequences corresponding to each group of amino acid units according to their degenerate codon; and
3) identifying nucleotide sequences obtained from step 2) that matches the nucleotide motif;
wherein the nucleotide motif consists of a nucleotide sequence of any one of SEQ ID NO: 95-116, preferably CUU, CUC or CAU, and
4) optionally, selecting the target ORF as the circularizing sequence of a circular RNA precursor.
317. A system for screening a target open reading frame (ORF) for a nucleotide motif, wherein the target open reading frame (ORF) is a nucleotide sequence encoding a target polypeptide, and the system comprises:
a) an amino acid extraction module, which is used for extracting amino acid units in the direction of N terminal to C terminal in the target polypeptide containing m amino acids, wherein each group of amino acid units comprise an amino acid with a number of n, with a step of 1 amino acid, sliding along the N terminal to the C terminal, and n is an integer and n≥2, m is an integer and ≥1;
b) a conversion module, which is used for determining the set of codon sequences of each amino acid unit, each set of codon sequences includes all degenerate codon sequence corresponding to each set of amino acid units; and
c) a target codon sequence filtering module, which is used for determining whether the target codon sequence contain a nucleotide sequence that exactly matches the motif,
wherein the nucleotide motif consists of a nucleotide sequence of any one of SEQ ID NO: 95-116, preferably CUU, CUC or CAU.
318. A method for screening a target internal ribosome entry site (IRES) for a nucleotide motif, comprising steps of:
1) identifying regions of non-conservative sequences of the target IRES by comparing the target IRES with the other IRES sequences from the same viral subgenus, through multiple sequence alignment tools, for example: Clustal Omega, or MUSCLE;
2) based on the results of multiple sequence alignment, determining a low-structure region of the target IRES through secondary structure prediction software, such as RNAfold, Mfold, RNAstructure or RNAalifold; and
3) editing 0, 1, or 2 bases in the low-structure region in the target IRES to match the nucleotide motif without affecting the translation initiation activity of said IRES;
wherein the nucleotide motif consists of a nucleotide sequence of any one of SEQ ID NO: 95-116, preferably CUU, CUC or CAU.
319. A system for screening a target internal ribosome entry site (IRES) for a nucleotide motif, wherein the system comprises:
a) a multi-sequence alignment module, which is used for comparing the target IRES with the other IRES sequences from the same viral subgenus to identify regions of non-conserved sequence;
b) a low-structure prediction module, which is used for determining a low-structure region within the target IRES according to the result of the multiple sequence alignments of the target IRES; and
c) a base editing module, which is used for editing the bases at any one or more positions in the region to obtain an edited region matching the nucleotide motif,
wherein the nucleotide motif consists of a nucleotide sequence of any one of SEQ ID NO: 95-116, preferably CUU, CUC or CAU.
320. A method for screening a target non-TIE functional element for a nucleotide motif, comprising a step of:
a) editing 0, 1, or 2 bases on the non-TIE functional element so that the non-TIE functional element sequence (such as a 3′UTR or replicon) comprises an edited region that matches the nucleotide motif whilst maintaining the non-TIE function;
wherein the nucleotide motif consists of a nucleotide sequence of any one of SEQ ID NO: 95-116, preferably CUU, CUC or CAU.
321. A system for screening a target region sequence in a non-TIE functional element sequence (such as a 3′UTR or replicon) for a nucleotide motif, wherein the system comprises:
a) a base editing module, which is used for editing the bases at any one or more positions in the non-TIE functional element sequence to obtain an edited region matching the nucleotide motif,
wherein the nucleotide motif consists of a nucleotide sequence of any one of SEQ ID NO: 95-116, preferably CUU, CUC or CAU.
322. A method for screening a target non-coding sequence for a nucleotide motif, comprising a step of:
1) editing 0, 1, or 2 bases on the non-coding sequence so that the non-coding sequence comprises an edited region that matches the nucleotide motif whilst maintaining the non-coding sequence's function;
wherein the nucleotide motif consists of a nucleotide sequence of any one of SEQ ID NO: 95-116, preferably CUU, CUC or CAU; and
2) optionally, selecting the target non-coding sequence as the circularizing sequence of a circular RNA precursor, wherein the target non-coding sequence is an endogenous circRNA.
323. A system for screening a target non-coding sequence for a nucleotide motif, wherein the target non-coding sequence is an endogenous circRNA, and the system comprises:
a) a base editing module, which is used for editing the bases at any one or more positions in the target non-coding sequence to obtain an edited region matching the nucleotide motif,
wherein the nucleotide motif consists of any one of SEQ ID NO: 95-116, preferably CUU, CUC or CAU.

### Examples

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner the present invention.

### Example 1. The Permutation Intron Exon (PIE) Design of Group I Intron

Permuted forms of group I introns in circular RNA precursors, which are interrupted by exon sequence, would fold into an active structure capable of splicing via the normal pathway. If the permuted intron-exon (PIE) sequence is active, the prediction is that a circular form of the ligated exon would be generated. Nonetheless, only a few permuted Group I Introns are used in the art that enables the self-splicing activities of Introns in the PIE system. Whether or not the Group I Introns will maintain the self-splicing activities after permuted at any site within the intron is unknown. Group I introns also possess at their core a conserved architecture formed by 10 paired segments (P1-P10) organized in three domains: a structural domain (P4-P6) that folds independently in the intron, a substrate domain (P1, P10), and a catalytic domain (P3, P7, and P8). These secondary structures create even more uncertainty for the selection of permutation sites within the self-splicing introns used for the PIE system. Which particular site is suitable as a site for PIE design is unknown.

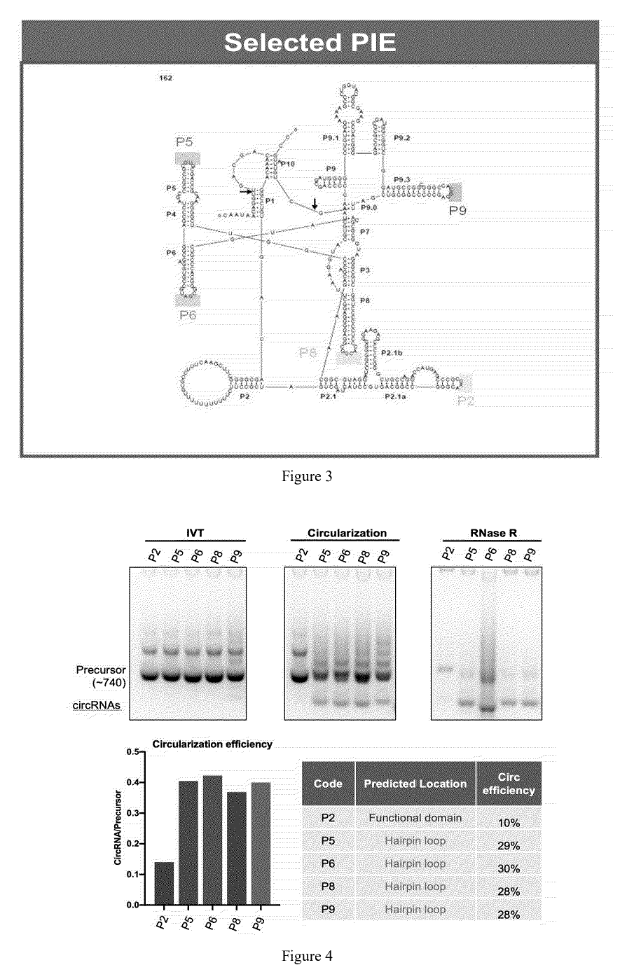

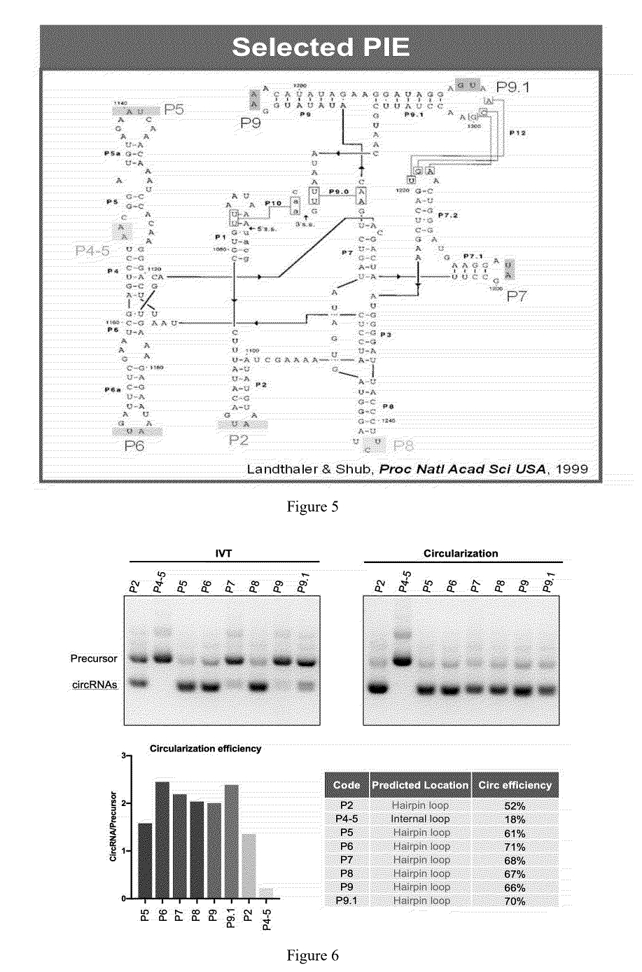

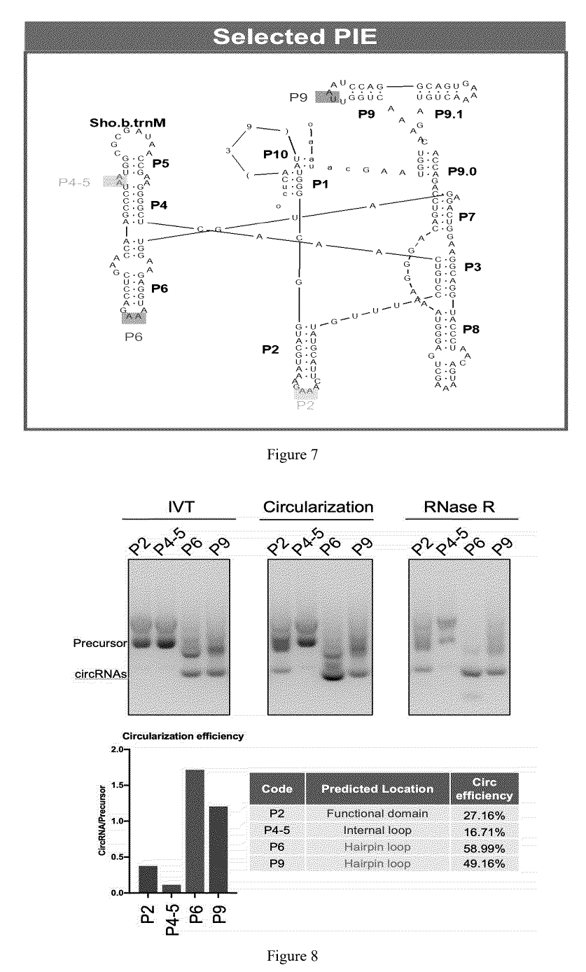

We designed a variety of permutation sites at loop area of P2, P4-5, P5, P6, P8, and P9 of Azoarcus Group I Intron (FIG. 1), at loop area of P2, P5, P6, P8, and P9 of Scytalidium dimidiatum Group I Intron (FIG. 3), and at loop area of P2, P4-5, P5, P6, P7, P8, P9, P9.1 of Staphylococcus phage Twort Group I Intron (FIG. 5), and P2, P4-5, P6 and P9 of Scytonema hofmanii Group I intron (FIG. 7).

### Example 2. Circularization Efficiency of Azoarcus Group I Intron with Different Permutation Sites

The basic principle is to connect exons E1-E2 end to end by molecular cloning to generate a continuous circular plasmid. The introns are cleaved by restrictive endonucleases to obtain linearized plasmids. Then, in vitro transcription is performed through the promoter upstream of the inverted 3′ intron to obtain a linear RNA containing the structure of 3′ intron-E2-E1-5′ intron. The conserved sequence of specific cleavage site of exon E1 is cleaved by the nucleophilic attack of the free 3′ hydroxyl of guanosine, exon E1 produces an exposed 3′ hydroxyl, and guanosine binds to the cleaved 5′ intron. Subsequently, the exposed 3′ hydroxyl of exon E1 attacks the conserved sequence between the 3′ intron and exon E2, the 3′ intron is excised, and exon E2 and E1 get connected. The reaction results in circular E1-E2 RNA (FIG. 14).

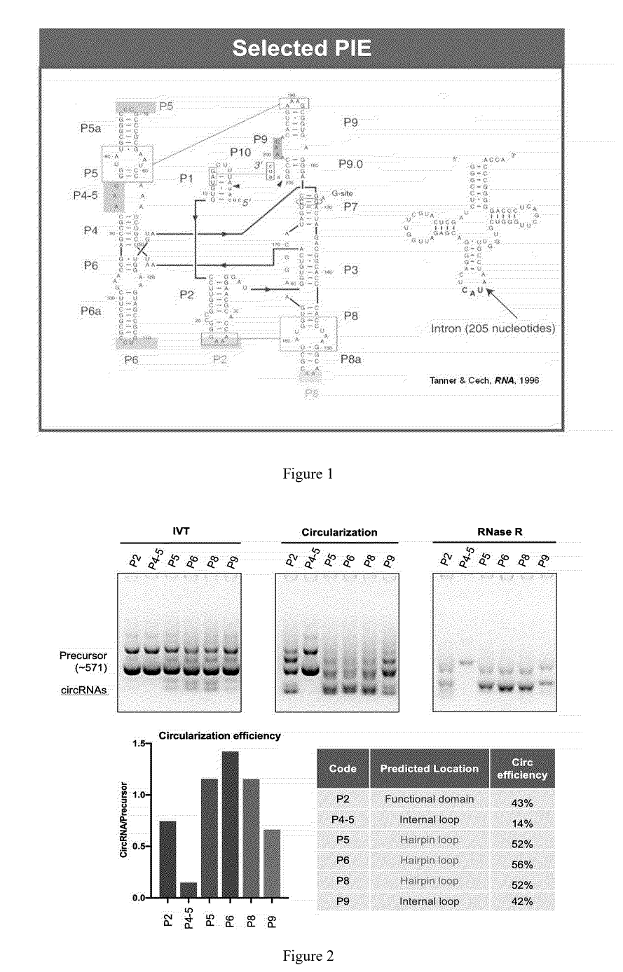

Taking circPOLR2A as the example, the homology arms, 3′ permuted intron-exon (PIE) construct of Azoarcus sp Group I Intron (3′intron and Exon2), circPOLR2A, 5′ permuted intron-exon (PIE) construct of Azoarcus sp Group I Intron (Exon1 and 5′intron) and homology arms were cloned into the plasmid containing a T7 RNA polymerase promoter by Gibson assembly. The circPOLR2A RNAs were produced by in vitro transcription (IVT) from the linearized plasmid template using the T7 RNA polymerase according to manufacturer's instruction, and then an additional step of incubation with 30 mM Mg²⁺ at 55° C. for 15 min. Circularization efficiency of constructs was measured by native agarose gel. Circularization efficiency of the 6 main permutation sites (P2, P4-5, P5, P6, P8, P9) of Azoarcus Group I Intron (Azop) are shown in FIG. 2 and Table 1. The permutation sites are varied from hairpin loop to internal loop. The permutation site of P4-5 and P9 domain which are located in internal loop showed the lower circularization efficiency. On the contrary, when the permutation site in the hairpin loop, such as P5, P6 and P8 domain, the circularization efficiency is relatively high. Although permutation site in P2 domain is a hairpin loop, it involved in forming the catalytic core of the Group I Intron, so disrupting the integrity of the P2 domain affect the splicing efficiency. These results indicate that the permutation site of Azoarcus Group I Intron (Azop) is better selected in hairpin loop than in functional domain or internal loop. Circularization efficiency is defined as the grayscale value for each of circRNA/precursor RNA or circRNA/(circRNA+precursor RNA). RNase R was used as a tool enzyme for verifying the circular structure of circular RNAs, and it can effectively degrade linear RNAs with free ends, but not circular RNAs.

TABLE 1

| | | Circularization efficiency |
|---|---|---|
| Code | Predicted Location | circRNA/(circRNA + precursor RNA) |
| P2 | Functional domain | 43% |
| P4-5 | Internal loop | 14% |
| P5 | Hairpin loop | 52% |
| P6 | Hairpin loop | 56% |
| P8 | Hairpin loop | 52% |
| P9 | Internal loop | 42% |

### Example 3. Circularization Efficiency of S. dimidiatum Group I Intron with Different Permutation Sites

Taking circPOLR2A as the example, the homology arms, 3′ permuted intron-exon (PIE) construct of Scytalidium dimidiatum Group I Intron (3′intron and Exon2), circPOLR2A, 5′ permuted intron-exon (PIE) construct of Scytalidium dimidiatum Group I Intron (Exon1 and 5′intron) and homology arms were cloned into the plasmid containing a T7 RNA polymerase promoter by Gibson assembly. The circPOLR2A RNAs were produced by in vitro transcription (IVT) from the linearized plasmid template using the T7 RNA polymerase according to manufacturer's instruction, and then an additional step of incubation with 30 mM Mg at 55° C. for 15 min. Circularization efficiency of constructs was measured by native agarose gel. Circularization efficiency of the 5 main permutation sites (P2, P5, P6, P8, P9) are shown in FIG. 4 and Table 2. The results show that the RNA circularization is inhibited when the permutation site is in function domain, such as P2 domain. On the contrary, when the permutation site is in the hairpin loop, such as P5, P6, P8 and P9 domain, the circularization efficiency is relatively high. It indicates that the permutation site of Scytalidium dimidiatum Group I Intron is better selected in hairpin loop than in functional domain. Circularization efficiency is defined as the grayscale value for each of circRNA/precursor RNA or circRNA/(circRNA+precursor RNA). RNase R was used as a tool enzyme for verifying the circular structure of circular RNAs, and it can effectively degrade linear RNAs with free ends, but not circular RNAs.

TABLE 2

| | | Circularization efficiency |
|---|---|---|
| Code | Predicted Location | circRNA/(circRNA + precursor RNA) |
| P2 | Functional domain | 10% |
| P5 | Hairpin loop | 29% |
| P6 | Hairpin loop | 30% |
| P8 | Hairpin loop | 28% |
| P9 | Hairpin loop | 28% |

### Example 4. Circularization Efficiency of Staphylococcus Phage Twort Group I Intron with Different Permutation Sites

Taking circPOLR2A as the example, the homology arms, 3′ permuted intron-exon (PIE) construct of Staphylococcus phage Twort Group I Intron (3′intron and Exon2), circPOLR2A, 5′ permuted intron-exon (PIE) construct of Staphylococcus phage Twort Group I Intron (Exon1 and 5′intron) and homology arms were cloned into the plasmid containing a T7 RNA polymerase promoter by Gibson assembly. The circPOLR2A RNAs were produced by in vitro transcription (IVT) from the linearized plasmid template using the T7 RNA polymerase according to manufacturer's instruction, and then an additional step of incubation with 30 mM Mg²⁺ at 55° C. for 15 min. Circularization efficiency of constructs was measured by native agarose gel. Circularization efficiency of the 8 main permutation sites (P2, P4-5, P5, P6, P7, P8, P9, P9.1) are shown in FIG. 6 and Table 3. The permutation sites are varied from hairpin loop to internal loop. The results show that the RNA circularization is inhibited when the permutation site is in internal loop, such as P4-5 domain. On the contrary, when the permutation site is in the hairpin loop, such as P2, P5, P6, P7, P8, P9 and P9.1 domain, the circularization efficiency is relatively high. It is indicated that the permutation site of Staphylococcus phage Twort Group I Intron is better selected in hairpin loop than in internal loop. And there's no significant difference whether the PIE site is symmetrical or asymmetrical from “stem”. Circularization efficiency is defined as the grayscale value for each of circRNA/precursor RNA or circRNA/(circRNA+precursor RNA). RNase R was used as a tool enzyme for verifying the circular structure of circular RNAs, and it can effectively degrade linear RNAs with free ends, but not circular RNAs.

TABLE 3

| | | Circularization efficiency |
|---|---|---|
| Code | Predicted Location | circRNA/(circRNA + precursor RNA) |
| P2 | Hairpin loop | 52% |
| P4-5 | Internal loop | 18% |
| P5 | Hairpin loop | 61% |
| P6 | Hairpin loop | 71% |
| P7 | Hairpin loop | 68% |
| P8 | Hairpin loop | 67% |
| P9 | Hairpin loop | 66% |
| P9.1 | Hairpin loop | 70% |

### Example 5. Circularization Efficiency of Scytonema-Hofmani tRNA fMet Group I Intron with Different Permutation Sites

Taking circPOLR2A as the example, the homology arms, 3′ permuted intron-exon (PIE) construct of Scytonema hofmanii tRNA fMet Group I Intron (3′intron and Exon2), circPOLR2A, 5′ permuted intron-exon (PIE) construct of Scytonema hofmanii tRNA fMet Group I Intron (Exon1 and 5′intron) and homology arms were cloned into the plasmid containing a T7 RNA polymerase promoter by Gibson assembly. The circPOLR2A RNAs were produced by in vitro transcription (IVT) from the linearized plasmid template using the T7 RNA polymerase according to manufacturer's instruction, and then an additional step of incubation with 30 mM Mg²⁺ at 55° C. for 15 min. Circularization efficiency of constructs was measured by native agarose gel. Circularization efficiency of the 4 main permutation sites (P2, P4-5, P6 and P9) of Scytonema hofmanii tRNA fMet Group I Intron (Sh) are shown in FIG. 8 and Table 4. The permutation sites are varied from hairpin loop to internal loop. The results show that the RNA circularization is inhibited when the permutation site is in functional domain or internal loop, such as P2 and P4-5 domain. On the contrary, when the permutation site is in the hairpin loop, such as P6 and P9 domain, the circularization efficiency is relatively high. It is indicated that the permutation site of Scytonema hofmanii tRNA fMet Group I Intron (Sh) is better selected in hairpin loop than in functional domain or internal loop. Circularization efficiency is defined as the grayscale value for each of circRNA/precursor RNA or circRNA/(circRNA+precursor RNA). RNase R was used as a tool enzyme for verifying the circular structure of circular RNAs, and it can effectively degrade linear RNAs with free ends, but not circular RNAs.

TABLE 4

| | | Circularization efficiency |
|---|---|---|
| Code | Predicted Location | circRNA/(circRNA + precursor RNA) |
| P2 | Functional | 27.16% |
| P4-5 | Internal loop | 16.71% |
| P6 | Hairpin loop | 58.99% |
| P9 | Hairpin loop | 49.16% |

### Example 6. Comparison of the Circularization Efficiency Between Staphylococcus Phage Twort Group I Intron and Anabaena Group I Intron

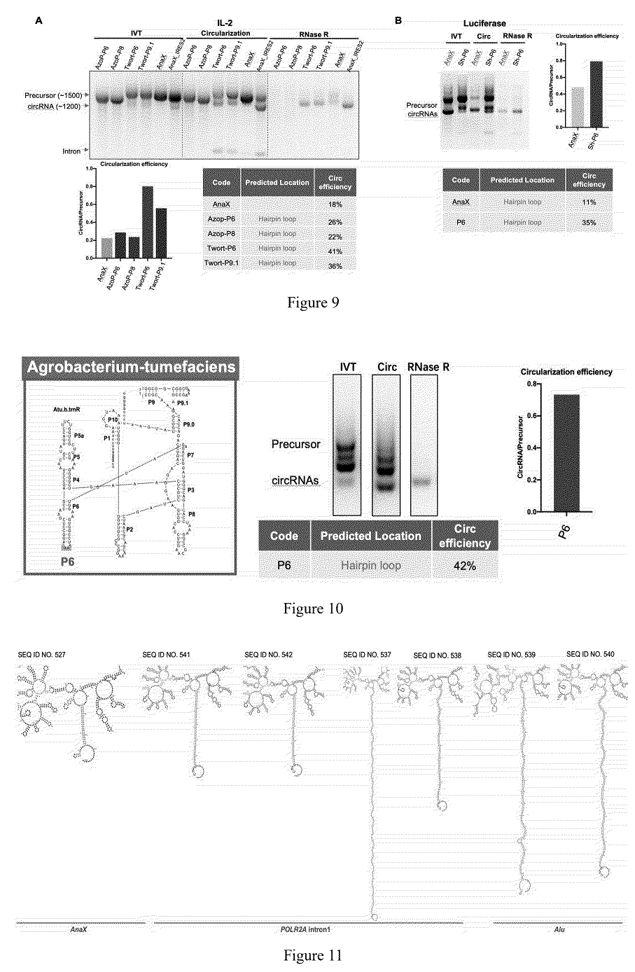

In order to compare the efficiency of Azoarcus Group I Intron, Twort Group I Intron, Sh Group I Intron and Anabaena Group I Intron for formulation of circRNA, we constructed Azoarcus, Twort, Sh and Anabaena plasmids containing T7 promoter, 5′ homology arm, 3′ permuted intron-exon (PIE) construct of Group I Intron (3′intron and Exon2), a Salivirus FHB IRES with IL2 sequence or a CVB2 IRES with Luciferase sequence, 5′permuted intron-exon (PIE) construct of Group I Intron (Exon1 and 5′intron) and 3′ homology arm to generate the circIL2 or circLuciferase using the group I intron circularization method that introduced above and the circularization efficiency was visualized on agarose gel (FIG. 9, Table 5 and Table6). The selection of PIE sites of Azoarcus Group I Intron, Twort Group I Intron and Sh Group I Intron (P6 and P8 from Azoarcus intron, P6 and P9-1 from Twort intron, P6 from Sh intron) were based on the cleanest and the highest circularization efficiency PIE sites for circPOLR2A cargo. Circularization efficiency is defined as the grayscale value for each of cricRNA/precursor RNA.

The result showed that compared with Anabaena Group I Intron (AnaX as set forth in SEQ ID NO:1), the circularization efficiency of Twort Group I Intron (SEQ ID NO:3) and Sh Group I Intron (SEQ ID NO:5) is significantly improved in the TL-2 (FIG. 9A) or Luciferase cargo (FIG. 9B), and Azoarcus Group I Intron (SEQ ID NO:2) has slight increase. RNase R was used as a tool enzyme for verifying the circular structure of circular RNAs, and it can effectively degrade linear RNAs with free ends, but not circular RNAs.

TABLE 5

| | | Circularization efficiency |
|---|---|---|
| | | circRNA/(circRNA + precursor |
| Code | Predicted Location | RNA) |
| AnaX | | 18% |
| Azop-P6 | Hairpin loop | 26% |
| Azop-P8 | Hairpin loop | 22% |
| Twort-P6 | Hairpin loop | 41% |
| Twort-P9.1 | Hairpin loop | 36% |

TABLE 6

| | | Circularization efficiency |
|---|---|---|
| Code | Predicted Location | circRNA/(circRNA + precursor RNA) |
| AnaX | | 11% |
| Sh-P6 | Hairpin loop | 35% |

### Example 7. Circularization Efficiency of Agrobacterium-Tumefaciens Group I Intron

We designed permutation sites at loop area of P6 domain of Agrobacterium-tumefaciens Group I Intron to test the circularization efficiency of the novel group I introns. circPOLR2A was generated in vitro by Agrobacterium-tumefaciens Group I Intron in a self-splicing reaction buffer (50 mM Tris-HCl, 10 mM MgCl₂, 1 mM DTT, pH 7.5). The circularization efficiency was visualized on agarose gel (FIG. 10, Table 7). The results show that Agrobacterium-tumefaciens Group I Intron can achieve circRNA formulation. The circularization efficiency is defined as the grayscale value for each of circRNA/precursor RNA or circRNA/(circRNA+precursor RNA). RNase R was used as a tool enzyme for verifying the circular structure of circular RNAs, and it can effectively degrade linear RNAs with free ends, but not circular RNAs.

TABLE 7

| | | Circularization efficiency |
|---|---|---|
| Code | Predicted Location | circRNA/(circRNA + precursor RNA) |
| P6 | Hairpin loop | 42% |

### Example 8. The Effect of Sequence Characteristic of Homology Arm Outside the Self-Splicing Intron Fragment on RNA Circularization

In order to improve the RNA circularization efficiency of group I intron, we tested the homology arm sequence outside the intron fragment of the AnaX system, including 1) using the endogenous non-complementary sequence (300 bp) from POLR2A intron 1 as 5′homology arm and fully complementary sequence (300 bp) of POLR2A intron 1 as 3′homology arm; 2) truncating the endogenous non-complementary sequence (300 bp) from POLR2A intron 1 to 100 bp; 3) using the natural AluSz sequence (285 nt) as 5′homology arm and AluYa5 sequence (174 nt) as 3′homology arm, and incomplete complementary pairing of partial AluSz and AluYa5 sequences; 4) modifying the natural AluYa5 (147 nt) to generate an artificial fully complementary Alu pair as homology arm; 5) truncating the endogenous non-complementary sequence (300 bp) from POLR2A intron 1 to 50 bp which is strong complementary; 6) truncating the endogenous non-complementary sequence (300 bp) from POLR2A intron 1 to 50 bp which is weak complementary (FIG. 11).

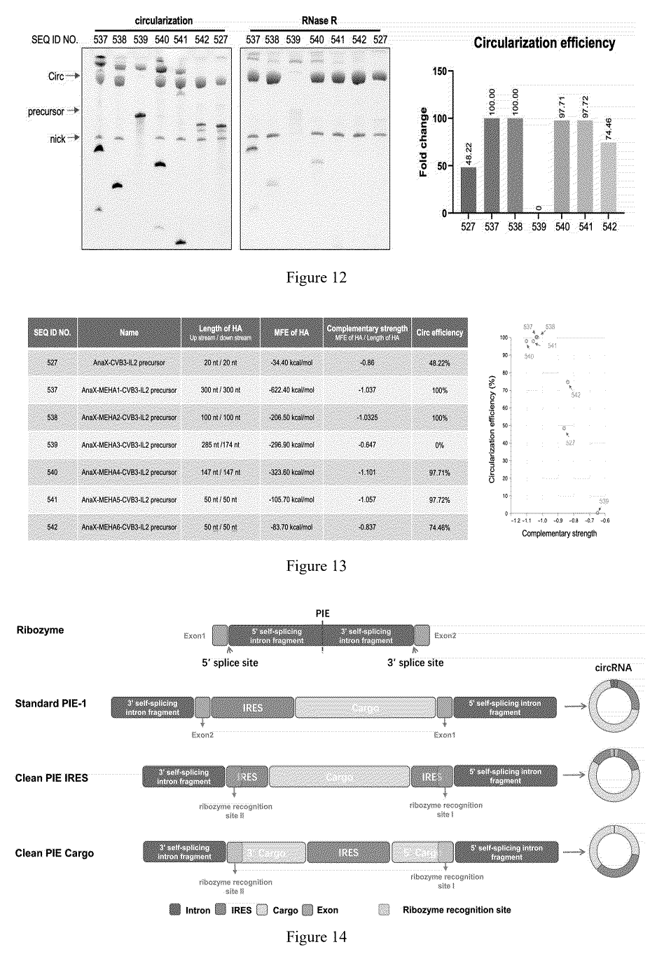

Next, we examined the effect of the retrofitted homology arm sequence outside the intron fragment on the circularization efficiency of the AnaX system. Taking the circularization of CVB3-IL2 RNAs as an example, the circularization efficiency was detected by denature PAGE gel. The results show that increasing the length of the complementary base-pairing sequence of the homology arm can improve the RNA circularization efficiency until the circularization efficiency reaches a level close to 100%. In addition, when the homology arm sequence was changed to highly complementary state, RNA circularization efficiency had obvious better effect. On the contrary, the non-complementary sequence reduced the RNA circularization efficiency (FIG. 12). The circularization efficiency is defined as the grayscale value for each of circRNA/(precursor RNA+circRNA) (FIG. 12). RNase R was used as a tool enzyme for verifying the circular structure of circular RNAs, and it can effectively degrade linear RNAs with free ends, but not circular RNAs. Then, we analyzed the average complementary base-pairing strength of each base on the homology arm, and the results show that the lower the minimum free energy of the base, the stronger the pairing strength, and the higher efficiency of circRNA formation (FIG. 13).

### Example 9. The Clean PIE Design for AnaX Group I Intron

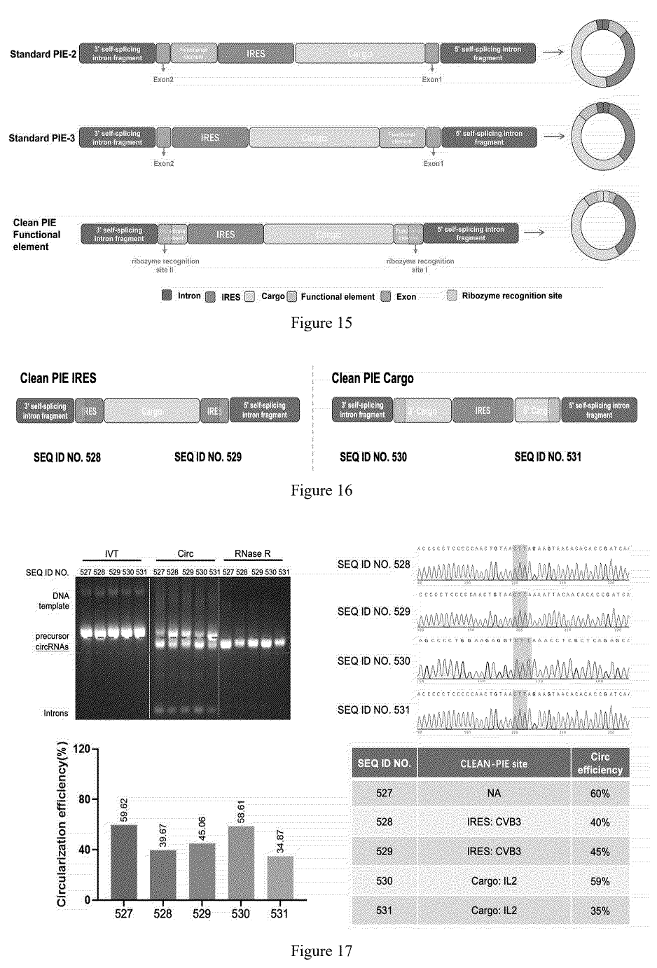

For AnaX group I intron, the 3 nucleotides motif (CUU, CUC or CAU) is the ribozyme recognition site I that pairs with the internal guide sequence (IGS) of the 5′ intron to form the P1 double-stranded region of the ribozyme, which can be used for the determination of the 5′ splicing site. We designed three types of precursors in which are the ribozyme recognition site I is in IRES, cargo and functional element respectively (FIGS. 14 and 15).

### 1. Design Clean PIE in IRES

Taking CVB3 as an example, we searched and identified the motif of CUU or CUC or CAU in the corresponding low structure region of CVB3 through RNAalifold, and performed minimal mutations on the flanking nucleotides of the motif to obtain the minimum base pairing numbers required for circularization. The precursor sequence was designed as follows from 5′ to 3′ direction (SEQ ID NO:528, 529, 535 and 536):
3′ self-splicing intron fragment, the sequence from downstream of motif CUU/CUC/CAU (motif not inclusive) to the 3′ end of CVB3 (SEQ ID NO:554,556), cargo sequence, the sequence from the 5′end of CVB3 to motif CUU/CUC/CAU (SEQ ID NO:555), and 5′ self-splicing intron fragment.

### 2. Design Clean PIE in Cargo

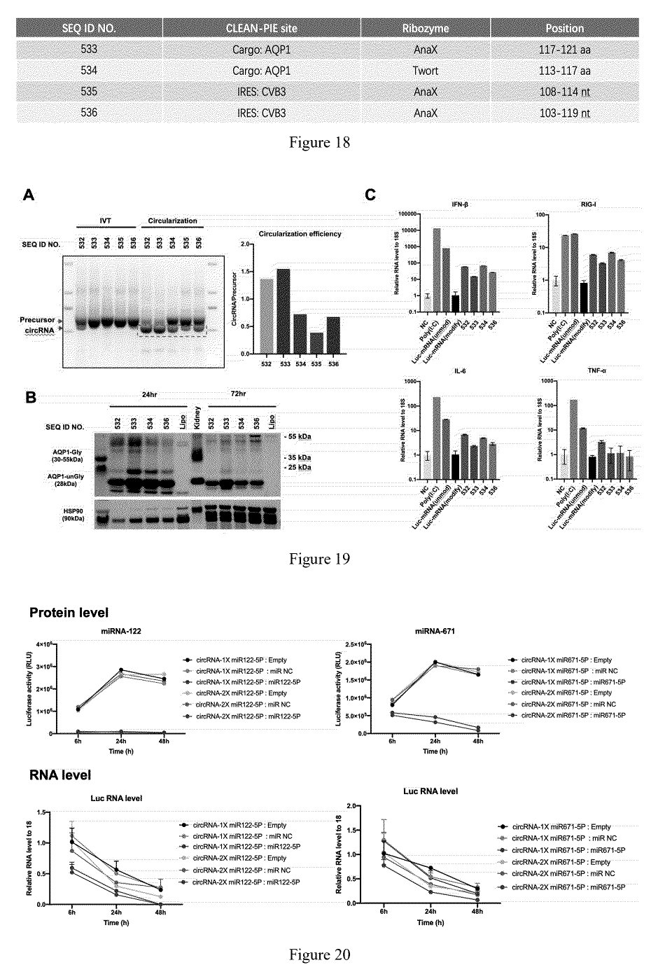

We searched and identified motif CUU or CUC or CAU through degenerate codon mutation method in the cargo (ORF). The precursor sequence was designed as follows from 5′ to 3′ direction:
3′ self-splicing intron fragment, the sequence from downstream of motif CUU/CUC/CAU (motif not inclusive) to the 3′ end of the ORF (SEQ ID NO:557, 559, 561), IRES sequence, the sequence from the 5′ end of the ORF to motif CUU/CUC/CAU (SEQ ID NO:558, 560, 562), and 5′ self-splicing intron fragment. A specific precursor was designed and the ORF of IL2 or AQP1 is used, wherein the corresponding sequences are SEQ ID NO. 530, 531 and 533 as shown in FIGS. 16 and 18.

### 3. Design Clean PIE in Functional Element

Taking 3′UTR as an example, we searched and identified CUU or CUC or CAU on the 3′UTR sequence. The precursor sequence was designed as follows from 5′ to 3′ direction:
3′ self-splicing intron fragment, the sequence from downstream of motif CUU/CUC/CAU (motif not inclusive) to the 3′ end of the UTR, IRES sequence, cargo sequence, the sequence from the 5′end of the UTR to motif CUU/CUC/CAU, and 5′ self-splicing intron fragment.

### Example 10. The RNA Circularization Efficiency of CLEAN-PIE

Precursors (SEQ ID NO. 528-531) for circular CVB3-IL2 RNA were designed according to the CLEAN-PIE strategy using the AnaX Group I Intron and were prepared by in vitro transcription from the linearized plasmid template using the T7 RNA polymerase according to manufacturer's instruction. The circularization efficiency was visualized on agarose gel and circular CVB3-IL2 prepared from traditional PIE method as the positive control (FIG. 17, Table 8). The results show that CLEAN-PIE designed in IRES or Cargo can both achieve circular CVB3-IL2 RNA, and the circularization efficiency was determined for different CLEAN-PIE designs. The circular RNAs were identified by Sanger Sequencing. Circularization efficiency is defined as the grayscale value for each of circRNA/(precursor RNA+circRNA). RNase R was used as a tool enzyme for verifying the circular structure of circular RNAs, and it can effectively degrade linear RNAs with free ends, but not circular RNAs.

TABLE 8

| SEQ ID NO. | CLEAN-PIE site | Circularization efficiency |
|---|---|---|
| 527 | NA | 60% |
| 528 | IRES: CVB3 | 40% |
| 529 | IRES: CVB3 | 45% |
| 530 | Cargo: IL2 | 59% |
| 531 | Cargo: IL2 | 35% |

### Example 11. Translation Efficiency and Immunogenicity of circRNA Obtained in CLEAN-PIE

To investigate the translation efficiency and immunogenicity of circRNA obtained from the CLEAN PIE, CLEAN-PIE were designed in CVB3 IRES or AQP1 cargo.

For CLEAN-PIE-IRES, we searched and identified CUU or CUC on CVB3. The precursor sequence was designed as follows from 5′ to 3′ direction (FIG. 18, SEQ ID NO.535 and 536):
3′ self-splicing intron fragment, the sequence from downstream of motif CUU (motif not inclusive) to the 3′ end of CVB3 (SEQ ID NO:554,556), cargo sequence, the sequence from the 5′end of CVB3 to motif CUU (SEQ ID NO:555), and 5′ self-splicing intron fragment.

For CLEAN-PIE-Cargo, we first searched and identified CUU or CUC through degenerate codon mutation method on AQP1. The precursor sequence was designed as follows from 5′ to 3′ direction (FIG. 18, SEQ ID NO.533):
3′ self-splicing intron fragment, the sequence from downstream of motif CUU (motif not inclusive) to the 3′ end of the ORF (SEQ ID NO:561), IRES sequence, the sequence from the 5′end of the ORF to motif CUU (SEQ ID NO:562), and 5′ self-splicing intron fragment.

Next, the CLEAN-PIE-circAQP1 was designed by following the above described strategies based on AnaX Group I intron (SEQ ID NO:1) and the precursors thereof were generated by in vitro transcription using the T7 RNA polymerase according to manufacturer's instruction. circAQP1 prepared from non-CLEAN PIE method was used as the positive control. Results show that the CLEAN-PIE designed in IRES or Cargo can achieve AQP1 RNA circularization with different efficiency (FIG. 19A). Then, we purified circAQP1 in vitro by RNase R treatment to achieve high purity, and transfected circAQP1 (200 ng/well) into human A253 cells in 12-well plate using Lipofectamine MessengerMAX™ (Thermo Fisher) RNA transfection reagent. After 24 hours and 72 hours of transfection, the cells were harvested, and the expression level of the AQP1 was determined using Western blot. HSP90 was used as the internal reference. The results show that circAQP1 produced from CLEARN-PIE had higher translation efficiency (FIG. 19B).

Further, we transfected the CLEAN-PIE-circAQP1 (200 ng/well) into human A549 cells in 12-well plate using Lipofectamine MessengerMAX™ (Thermo Fisher) RNA transfection reagent to detect the immunogenicity. After 6 hours of transfection, the cells were harvested, and the expression levels of the cytokines, including IFNβ, TNFα, IL6 and RIG-I, were determined using quantitative RT-PCR and normalized to 18S rRNA. The results show that compared to circAQP1 prepared from traditional PIE method, the purified circAQP1 prepared via CLEAN-PIE circularization methods showed no significant differences in the expression level of IFNβ, TNFα, IL6 and RIG-I (FIG. 19C).

These data demonstrated that the circAQP1 prepared in vitro using CLEAN-PIE circularization methods do not cause significant immune responses. IFNβ, TNFα, IL6 and RIG-I are commonly used as marker genes for the measurement of cellular immunogenicity, and the positive control Poly (I:C) is a commonly used long double-stranded RNAto mimic virus infection at circular level. mRNA is used as a reference sequence (SEQ ID NO:624).

### Example 12. In Vivo Expression of circRNA-hAQP1 Injection

To evaluate the in vivo expression of AQP1, female wild Sprague Dawley rats of an age of 6 weeks were administrated with 100 μL of PBS (Sangon Biotech) or LNP encapsulated CLEAN-PIE-circAQP1 prepared in Example 11 at 0.5 μg, 1.5 μg, or 5 μg into the left submandibular salivary gland via retrograde ductal infusion. On D3 and D16 post-treatment,in each group, both administrated and non-administrated submandibular glands were collected from three animals for immunohistochemistry (IHC). A specific antibody against hAQP1 (Abcam) was used for IHC staining.

As shown in FIGS. 22 and 23, higher signals were detected in the groups of higher doses (1.5 and 5 μg) as compared to the 0.5 g dose group. On day 16, hAQP1 expression was still detectable in the animals injected with 5 g in contrast to the animals treated with lower dose levels.

Retrograde ductal infusion with LNP encapsulated CLEAN-PIE-circAQP1 increases in situ expression level of hAQP1 in the administrated submandibular glands rather than the non-administrated one. The semi-quantitative analysis revealed a trend for dose responses with higher expression at higher doses. The duration of in situ expression in administrated submandibular glands lasted from day 3 up to day 16.

### Example 13. The CLEAN-PIE Design for Twort Group I Intron

We designed CLEAN-PIE precursor using Twort Group I introns. Taking circCVB3-AQP1 as an example, we searched the CAU through degenerate codon mutation method. The precursor sequence was designed as follows from 5′ to 3′ direction (FIG. 18, SEQ ID NO. 534):
3′ self-splicing intron fragment, the sequence from downstream of motif CAU (motif not inclusive) to the 3′ end of AQP1 (SEQ ID NO:563), IRES sequence, the sequence from the 5′end of the ORF of AQP1 to motif CAU (SEQ ID NO:564), and 5′ self-splicing intron fragment.

Next, circCVB3-AQP1 was generate by in vitro transcription using the T7 RNA polymerase according to manufacturer's instruction and then incubation with 30 mM Mg²⁺ at 55° C. for 15 min for self-splicing circularization. Results show that the CLEAN-PIE designed in cargo can achieve RNA circularization (FIG. 19A). Then, we purified the circCVB3-AQP1 in vitro by RNase R treatment to achieve high purity, and transfected circCVB3-AQP1 (200 ng/well) into human A253 cells in 12-well plate using Lipofectamine MessengerMAX™ (Thermo Fisher) RNA transfection reagent. After 24 hours and 72 hours of transfection, the cells were harvested, and the expression levels of the AQP1 were determined using western blot and HSP90 was used as the internal reference. The results showed that circCVB3-AQP1 produced from CLEARN-PIE has higher translation efficiency (FIG. 19B). It indicates that Twort group I intron can be used in CLEAN PIE designs to achieve RNA circularization and circRNA translation.

Further, we transfected the CLEAN-PIE circCVB3-AQP1 (200 ng/well) into human A549 cells in 12-well plate using Lipofectamine MessengerMAX™ (Thermo Fisher) RNA transfection reagent to detect the immunogenicity. After 6 hours of transfection, the cells were harvested, and the expression levels of the cytokines, including IFNβ, TNFα, IL6 and RIG-I, were determined using quantitative RT-PCR and normalized to 18S rRNA. The results show that compared to circCVB3-AQP1 prepared from traditional PIE method, the purified circCVB3-AQP1 prepared via CLEAN-PIE circularization methods show no significant differences in the expression level of IFNβ, TNFα, IL6 and RIG-I (FIG. 19C). These data demonstrate that the circCVB3-AQP1 generated by CLEAN-PIE design of Twort group I intron does not cause significant immune responses. IFNβ, TNFα, IL6 and RIG-I are commonly used as marker genes for the measurement of cellular immunogenicity, and the positive control Poly (I:C) is a commonly used long double-stranded RNA to mimic virus infection at circular level.

### Example 14. The Regulation of circRNA Expression in Cells by miRNA

MicroRNA (miRNA) are small non-coding RNA molecules containing 20 to 23 nucleotides. The main function of miRNAs is to induce mRNA degradation and translational repression by forming complementary base-pairing with the 3′ untranslated region (3′UTR) of target mRNAs. In this example, we demonstrate the regulation of circRNA expression in cells by introducing miRNA binding sites into circRNAs.

We inserted 1 (1× circRNA) or 2 (2× circRNA) tandem miRNA binding sites targeting miRNA-122 or miR-671 respectively into the circRNA constructs that contain the IRES element and luciferase coding sequence. Then circRNAs containing miRNA binding sites were transfected into HEK293 cells overexpressing miRNA-122 or miR-671. The protein expression level and RNA level of each circRNA were detected after transfection 6 h, 24 h and 48h, respectively.

The results show that the luciferase expression levels are significantly inhibited when miRNA-122 or miR-671 binding sites were inserted into circRNAs (FIG. 20). In addition, the amount of circRNAs containing miRNA-122 or miR-671 binding sites was also significantly decreased compared to the control group (FIG. 20). The above results demonstrate that miRNA can regulate circRNA expression by degrading circular RNA, and thus realize the cell type- or tissue-specific expression of circular RNA.

### Example 15. Regulation of circRNA Translation by UTRs

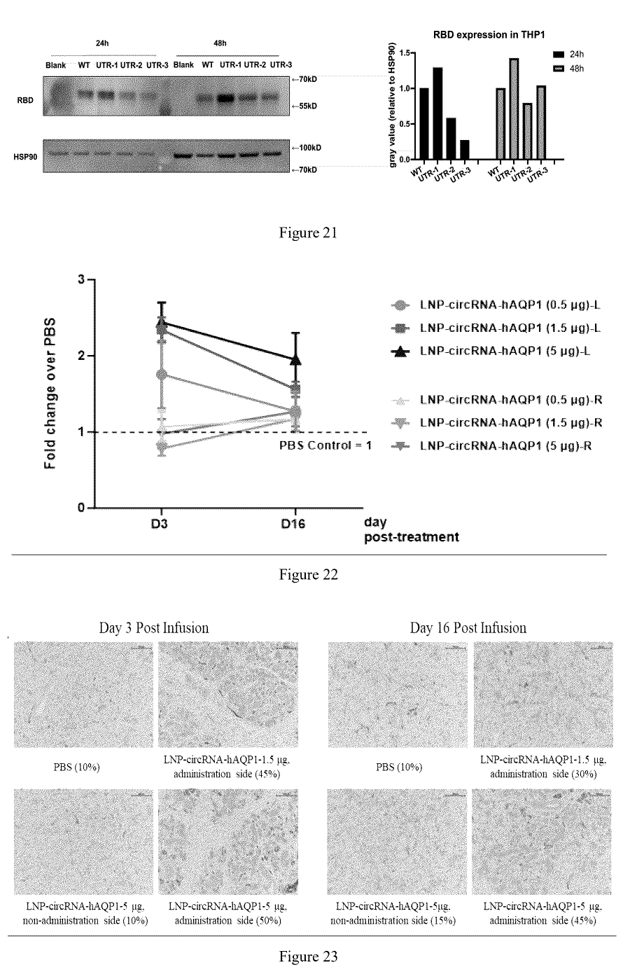

Untranslated regions (UTR) flanking the ORF can bind with regulatory proteins and thus affect the protein translational efficiency. In this example, we describe the regulation of circRNA translation by introducing UTRs into the circular RNA. We inserted the UTR element into the 3′ end of RBD ORF sequence in the circular RNA and transfected the circular RNA into the THP1 cells, then detected the expression level after transfection 24 h and 48 h by western blot, respectively. The results demonstrate that UTRs of different sequences may have different effects. UTR-1 enhanced the translation of circRNA, while UTR-2 and UTR-3 showed inhibitory effects (FIG. 21).

While the invention is described in conjunction with the enumerated embodiments, it will be understood that they are not intended to limit the invention to those embodiments. The invention is intended to cover all alternatives, modifications, and equivalents that may be included within the scope of the present invention. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

All references including patents, patent applications and publications cited in the present application are incorporated herein by reference in their entirety, as if each of them is individually incorporated. Further, it would be appreciated that one skilled in the art could make various changes or modifications to the invention without departing from the scope of the invention defined by the appended claims below. Accordingly, the present invention is not intended to be limited to the disclosed embodiments. Rather the present invention is intended to cover the disclosed embodiments as well as others falling within the scope and spirit of the invention to the fullest extent permitted in view of this disclosure and the inventions defined by the claims appended herein below.

Sequences mentioned herein are listed as below:

| SEQ | | |
|---|---|---|
| ID | | |
| NO. | Sequence | Description |
| 1 | AGACGCUACGGACUUAAAUAAUUGAGCCUUAAAGAAGAAAUUCUUUAAGUGGAUGCUCUCAAACUCA | GROUP I |
| | GGGAAACCUAAAUCUAGUUAUAGACAAGGCAAUCCUGAGCCAAGCCGAAGUAGUAAUUAGUAAAAAC | INTRON, |
| | AAUAGAUGACUUACAACUAAUCGGAAGGUGCAGAGACUCGACGGGAGCUACCCUAACGUCAAGACGA | Anabaena sp. |
| | GGGUAAAGAGAGAGUCCAAUUCUCAAAGCCAAUAGGCAGUAGCGAAAGCUGCAAGAGAAUGAAAAUC | YBS01 |
| | CGUUGA | |
| 2 | GAGCAGCGGACUCAUAUUUCGAUGUGCCUUGCGCCGGGAAACCACGCAAGGGAUGGUGUCAAAUUCG | GROUP I |
| | GCGAAACCUAAGCGCCCGCCCGGGCGUAUGGCAACGCCGAGCCAAGCUUCGGCGCCUGCGCCGAUGA | INTRON |
| | AGGUGUAGAGACUAGACGGCACCCACCUAAGGCAAACGCUAUGGUGAAGGCAUAGUCCAGGGAGUGG | Azoarcus |
| | CGAAAGUCACACAAACCGGAAUCCGUUGGUGCUG | olearius |
| | | BH72 |
| 3 | ACUACUGAAAGCAUAAAUAAUUGUGCCUUUAUACAGUAAUGUAUAUCGAAAAAUCCUCUAAUUCAGG | GROUP I |
| | GAACACCUAAACAAACUAAGAUGUAGGCAAUCCUGAGCUAAGCUCUUAGUAAUAAGAGAAAGUGCAA | INTRON, |
| | CGACUAUUCCGAUAGGAAGUAGGGUCAAGUGACUCGAAAUGGGGAUUACCCUUCUAGGGUAGUGAUA | Staphylococcus |
| | UAGUCUGAACAUAUAUGGAAACAUAUAGAAGGAUAGGAGUAACGAACCUAUUCGUAACAUAAUUGAA | phage |
| | CUUUUAGUUAU | Twort |
| 4 | AACAGGUUGACGACAACAGGCCUGUGACAGCGGGGCUUCGAACUUUCCGUGUUUUUUUUUCUUCCGC | GROUP I |
| | UAGUCGAUCAUCCGUGACGGCCGGGCAAGCGCCCGAGUACCAGGACCGUCGGGCCCCCGAGAAGGGG | INTRON, |
| | GGGCCUAGGAUGCGGCAAGACGACCCGGUUCGGGGAACGCCAGCGUGCGCUGGCCGAUCCCGAGGCG | Scytalidium |
| | AGGUGCCGUAGCGGGCACCCGUCGUAACGCGCGGUAGGGCGUCGGUCCCCCCUCCCACGGCGGAGGG | dimidiatum |
| | GAGGCUUAAGGUACGUGCUAAACCCCCAGCGAUGGGGCCUGUAGGAAAAGCCCUGGUACGGCGAAGC | |
| | CUACGGGGACCGCCCGAUGGCGGUCGGAUGCCGGCGGGCCACGGAGCCCCCGGCGUCGAUUGCUGUU | |
| | A | |
| 5 | GGGCUCAACGAGUAAGAUUGACUAAACGCUUAAAAGUUAUUCUUACUAUGGGCGGUACGUAAAGAAA | GROUP I |
| | CUUACGUAUGUUUACCUGUCAAACUCGGGGAAGCCAAUAGCGCGGUAAUCCCGAACCAAGCUCCAGA | INTRON, |
| | AAUGGAGAAGGUGUAGAGACUGGAAGGCAGGUACCCUAACAGUAAAGCUGAGGGUAAAGGGACAGUC | Scytonema- |
| | CAGACCACAAACUGGUUAAUCCAGGCAGUGAAAACUGUAGAUGGUAAGCAUAACCC | hofmani tRNA |
| | | fMet |
| 6 | CAGGAUUCCUAAGGUAAAUUGGGGGUUGCGCCCGGAAACGACGCAAUCGAUCUGCUCAAAGUCGGGG | GROUP I |
| | AAAGCUUCGCUGGUCUUCCAGUAUGCCAAUCCCGAGCCAAGCUCCGGAGAAAUCCGGUGAAGGUGUA | INTRON, |
| | GAGACUGGAUGGGCAGCACCUAAAGGCCGCAAGGUCCACGGUGAAGGGACAGUCCAGACCACGAACG | Agrobacterium- |
| | CCCUUAUCCGAUUGAGAUGGGCGGCGGCGAAAGCCGAAGCGGUAUGAAUCCUGGGG | tumefaciens |
| 7 | AACAAUAGAUGACUUACAACUAAUCGGAAGGUGCAGAGACUCGACGGGAGCUACCCUAACGUCAAGA | 3′ self- |
| | CGAGGGUAAAGAGAGAGUCCAAUUCUCAAAGCCAAUAGGCAGUAGCGAAAGCUGCAAGAGAAUG | splicing |
| | | intron |
| | | fragment |
| | | derived from |
| | | Anabaena |
| | | pre-tRNA-Leu |
| | | gene GROUP I |
| | | INTRON |
| 8 | AAAUAAUUGAGCCUUAAAGAAGAAAUUCUUUAAGUGGAUGCUCUCAAACUCAGGGAAACCUAAAUCU | 5′ self- |
| | AGUUAUAGACAAGGCAAUCCUGAGCCAAGCCGAAGUAGUAAUUAGUAA | splicing |
| | | intron |
| | | fragment |
| | | derived from |
| | | Anabaena |
| | | pre-tRNA-Leu |
| | | gene GROUP I |
| | | INTRON |
| 9 | AACCACGCAAGGGAUGGUGUCAAAUUCGGCGAAACCUAAGCGCCCGCCCGGGCGUAUGGCAACGCCG | 3′ self- |
| | AGCCAAGCUUCGGCGCCUGCGCCGAUGAAGGUGUAGAGACUAGACGGCACCCACCUAAGGCAAACGC | splicing |
| | UAUGGUGAAGGCAUAGUCCAGGGAGUGGCGAAAGUCACACAAACCGG | intron |
| | | fragment |
| | | derived from |
| | | Azoarcus |
| | | olearius |
| | | BH72 GROUP I |
| | | INTRON-PIE-2 |
| 10 | AUUUCGAUGUGCCUUGCGCCGGGA | 5′ self- |
| | | splicing |
| | | intron |
| | | fragment |
| | | derived from |
| | | Azoarcus |
| | | olearius |
| | | BH72 GROUP I |
| | | INTRON-PIE-2 |
| 11 | ACGCCGAGCCAAGCUUCGGCGCCUGCGCCGAUGAAGGUGUAGAGACUAGACGGCACCCACCUAAGGC | 3′ self- |
| | AAACGCUAUGGUGAAGGCAUAGUCCAGGGAGUGGCGAAAGUCACACAAACCGG | splicing |
| | | intron |
| | | fragment |
| | | derived from |
| | | Azoarcus |
| | | olearius |
| | | BH72 GROUP I |
| | | INTRON-PIE- |
| | | 4-5 |
| 12 | AUUUCGAUGUGCCUUGCGCCGGGAAACCACGCAAGGGAUGGUGUCAAAUUCGGCGAAACCUAAGCGC | 5′ self- |
| | CCGCCCGGGCGUAUGGCA | splicing |
| | | intron |
| | | fragment |
| | | derived from |
| | | Azoarcus |
| | | olearius |
| | | BH72 GROUP I |
| | | INTRON-PIE- |
| | | 4-5 |
| 13 | CCGGGCGUAUGGCAACGCCGAGCCAAGCUUCGGCGCCUGCGCCGAUGAAGGUGUAGAGACUAGACGG | 3′ self- |
| | CACCCACCUAAGGCAAACGCUAUGGUGAAGGCAUAGUCCAGGGAGUGGCGAAAGUCACACAAACCGG | splicing |
| | | intron |
| | | fragment |
| | | derived from |
| | | Azoarcus |
| | | olearius |
| | | BH72 GROUP I |
| | | INTRON-PIE-5 |
| 14 | AUUUCGAUGUGCCUUGCGCCGGGAAACCACGCAAGGGAUGGUGUCAAAUUCGGCGAAACCUAAGCGC | 5′ self- |
| | CCGC | splicing |
| | | intron |
| | | fragment |
| | | derived from |
| | | Azoarcus |
| | | olearius |
| | | BH72 GROUP I |
| | | INTRON-PIE-5 |
| 15 | UGCGCCGAUGAAGGUGUAGAGACUAGACGGCACCCACCUAAGGCAAACGCUAUGGUGAAGGCAUAGU | 3′ self- |
| | CCAGGGAGUGGCGAAAGUCACACAAACCGG | splicing |
| | | intron |
| | | fragment |
| | | derived from |
| | | Azoarcus |
| | | olearius |
| | | BH72 GROUP I |
| | | INTRON-PIE-6 |
| 16 | AUUUCGAUGUGCCUUGCGCCGGGAAACCACGCAAGGGAUGGUGUCAAAUUCGGCGAAACCUAAGCGC | 5′ self- |
| | CCGCCCGGGCGUAUGGCAACGCCGAGCCAAGCUUCGGCGCC | splicing |
| | | intron |
| | | fragment |
| | | derived from |
| | | Azoarcus |
| | | olearius |
| | | BH72 GROUP I |
| | | INTRON-PIE-6 |
| 17 | ACGCUAUGGUGAAGGCAUAGUCCAGGGAGUGGCGAAAGUCACACAAACCGG | 3′ self- |
| | | splicing |
| | | intron |
| | | fragment |
| | | derived from |
| | | Azoarcus |
| | | olearius |
| | | BH72 GROUP I |
| | | INTRON-PIE-8 |
| 18 | AUUUCGAUGUGCCUUGCGCCGGGAAACCACGCAAGGGAUGGUGUCAAAUUCGGCGAAACCUAAGCGC | 5′ self- |
| | CCGCCCGGGCGUAUGGCAACGCCGAGCCAAGCUUCGGCGCCUGCGCCGAUGAAGGUGUAGAGACUAG | splicing |
| | ACGGCACCCACCUAAGGCAA | intron |
| | | fragment |
| | | derived from |
| | | Azoarcus |
| | | olearius |
| | | BH72 GROUP I |
| | | INTRON-PIE-8 |
| 19 | AAACCGG | 3′ self- |
| | | splicing |
| | | intron |
| | | fragment |
| | | derived from |
| | | Azoarcus |
| | | olearius |
| | | BH72 GROUP I |
| | | INTRON-PIE-9 |
| 20 | AUUUCGAUGUGCCUUGCGCCGGGAAACCACGCAAGGGAUGGUGUCAAAUUCGGCGAAACCUAAGCGC | 5′ self- |
| | CCGCCCGGGCGUAUGGCAACGCCGAGCCAAGCUUCGGCGCCUGCGCCGAUGAAGGUGUAGAGACUAG | splicing |
| | ACGGCACCCACCUAAGGCAAACGCUAUGGUGAAGGCAUAGUCCAGGGAGUGGCGAAAGUCACAC | intron |
| | | fragment |
| | | derived from |
| | | Azoarcus |
| | | olearius |
| | | BH72 GROUP I |
| | | INTRON-PIE-9 |
| 21 | AAUGUAUAUCGAAAAAUCCUCUAAUUCAGGGAACACCUAAACAAACUAAGAUGUAGGCAAUCCUGAG | 3′ self- |
| | CUAAGCUCUUAGUAAUAAGAGAAAGUGCAACGACUAUUCCGAUAGGAAGUAGGGUCAAGUGACUCGA | splicing |
| | AAUGGGGAUUACCCUUCUAGGGUAGUGAUAUAGUCUGAACAUAUAUGGAAACAUAUAGAAGGAUAGG | intron |
| | AGUAACGAACCUAUUCGUAACAUAAUUG | fragment |
| | | derived from |
| | | Staphylococcusus |
| | | phage |
| | | Twort GROUP |
| | | I INTRON- |
| | | PIE-2 |
| 22 | AAAUAAUUGUGCCUUUAUACAGU | 5′ self- |
| | | splicing |
| | | intron |
| | | fragment |
| | | derived from |
| | | Staphylococcus |
| | | phage |
| | | Twort GROUP |
| | | I INTRON- |
| | | PIE-2 |
| 23 | AUCCUGAGCUAAGCUCUUAGUAAUAAGAGAAAGUGCAACGACUAUUCCGAUAGGAAGUAGGGUCAAG | 3′ self- |
| | UGACUCGAAAUGGGGAUUACCCUUCUAGGGUAGUGAUAUAGUCUGAACAUAUAUGGAAACAUAUAGA | splicing |
| | AGGAUAGGAGUAACGAACCUAUUCGUAACAUAAUUG | intron |
| | | fragment |
| | | derived from |
| | | Staphylococcus |
| | | phage |
| | | Twort GROUP |
| | | I INTRON- |
| | | PIE-4-5 |
| 24 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCA | splicing |
| | | intron |
| | | fragment |
| | | derived from |
| | | Staphylococcus |
| | | phage |
| | | Twort GROUP |
| | | I INTRON- |
| | | PIE-4-5 |
| 25 | AAGAUGUAGGCAAUCCUGAGCUAAGCUCUUAGUAAUAAGAGAAAGUGCAACGACUAUUCCGAUAGGA | 3′ self- |
| | AGUAGGGUCAAGUGACUCGAAAUGGGGAUUACCCUUCUAGGGUAGUGAUAUAGUCUGAACAUAUAUG | splicing |
| | GAAACAUAUAGAAGGAUAGGAGUAACGAACCUAUUCGUAACAUAAUUG | intron |
| | | fragment |
| | | derived from |
| | | Staphylococcus |
| | | phage |
| | | Twort GROUP |
| | | I INTRON- |
| | | PIE-5 |
| 26 | AAAUAAUUGUGCCUUUAUACAGUAAUGUAUAUCGAAAAAUCCUCUAAUUCAGGGAACACCUAAACAA | 5′ self- |
| | ACU | splicing |
| | | intron |
| | | fragment |
| | | derived from |
| | | Staphylococcus |
| | | phage |
| | | Twort GROUP |
| | | I INTRON- |
| | | PIE-5 |
| 27 | AAUAAGAGAAAGUGCAACGACUAUUCCGAUAGGAAGUAGGGUCAAGUGACUCGAAAUGGGGAUUACC | 3′ self- |
| | CUUCUAGGGUAGUGAUAUAGUCUGAACAUAUAUGGAAACAUAUAGAAGGAUAGGAGUAACGAACCUA | splicing |
| | UUCGUAACAUAAUUG | intron |
| | | fragment |
| | | derived from |
| | | Staphylococcus |
| | | phage |
| | | Twort GROUP |
| | | I INTRON- |
| | | PIE-6 |
| 28 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCAAUCCUGAGCUAAGCUCUUAGU | splicing |
| | | intron |
| | | fragment |
| | | derived from |
| | | Staphylococcus |
| | | phage |
| | | Twort GROUP |
| | | I INTRON- |
| | | PIE-6 |
| 29 | UAGGAAGUAGGGUCAAGUGACUCGAAAUGGGGAUUACCCUUCUAGGGUAGUGAUAUAGUCUGAACAU | 3′ self- |
| | AUAUGGAAACAUAUAGAAGGAUAGGAGUAACGAACCUAUUCGUAACAUAAUUG | splicing |
| | | intron |
| | | fragment |
| | | derived from |
| | | Staphylococcus |
| | | phage |
| | | Twort GROUP |
| | | I INTRON- |
| | | PIE-7 |
| 30 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCAAUCCUGAGCUAAGCUCUUAGUAAUAAGAGAAAGUGCAACGACUAUUCCGA | splicing |
| | | intron |
| | | fragment |
| | | derived from |
| | | Staphylococcus |
| | | phage |
| | | Twort GROUP |
| | | I INTRON- |
| | | PIE-7 |
| 31 | CUAGGGUAGUGAUAUAGUCUGAACAUAUAUGGAAACAUAUAGAAGGAUAGGAGUAACGAACCUAUUC | 3′ self- |
| | GUAACAUAAUUG | splicing |
| | | intron |
| | | fragment |
| | | derived from |
| | | Staphylococcus |
| | | phage |
| | | Twort GROUP |
| | | I INTRON- |
| | | PIE-8 |
| 32 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCAAUCCUGAGCUAAGCUCUUAGUAAUAAGAGAAAGUGCAACGACUAUUCCGAUA | splicing |
| | GGAAGUAGGGUCAAGUGACUCGAAAUGGGGAUUACCCUU | intron |
| | | fragment |
| | | derived from |
| | | Staphylococcus |
| | | phage |
| | | Twort GROUP |
| | | I INTRON- |
| | | PIE-8 |
| 33 | AACAUAUAGAAGGAUAGGAGUAACGAACCUAUUCGUAACAUAAUUG | 3′ self- |
| | | splicing |
| | | intron |
| | | fragment |
| | | derived from |
| | | Staphylococcus |
| | | phage |
| | | Twort GROUP |
| | | I INTRON- |
| | | PIE-9 |
| 34 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCAAUCCUGAGCUAAGCUCUUAGUAAUAAGAGAAAGUGCAACGACUAUUCCGAUA | splicing |
| | GGAAGUAGGGUCAAGUGACUCGAAAUGGGGAUUACCCUUCUAGGGUAGUGAUAUAGUCUGAACAUAU | intron |
| | AUGGA | fragment |
| | | derived from |
| | | Staphylococcus |
| | | phage |
| | | Twort GROUP |
| | | I INTRON- |
| | | PIE-9 |
| 35 | UAACGAACCUAUUCGUAACAUAAUUG | 3′ self- |
| | | splicing |
| | | intron |
| | | fragment |
| | | derived from |
| | | Staphylococcus |
| | | phage |
| | | Twort GROUP |
| | | I INTRON- |
| | | PIE-9.1 |
| 36 | AAAUAAUUGUGCCUUUAUACAGUAAUGUAUAUCGAAAAAUCCUCUAAUUCAGGGAACACCUAAACAA | 5′ self- |
| | ACUAAGAUGUAGGCAAUCCUGAGCUAAGCUCUUAGUAAUAAGAGAAAGUGCAACGACUAUUCCGAUA | splicing |
| | GGAAGUAGGGUCAAGUGACUCGAAAUGGGGAUUACCCUUCUAGGGUAGUGAUAUAGUCUGAACAUAU | intron |
| | AUGGAAACAUAUAGAAGGAUAGGAG | fragment |
| | | derived from |
| | | Staphylococcus |
| | | phage |
| | | Twort GROUP |
| | | I INTRON- |
| | | PIE-9.1 |
| 37 | GCGCCCGAGUACCAGGACCGUCGGGCCCCCGAGAAGGGGGGGCCUAGGAUGCGGCAAGACGACCCGG | 3′ self- |
| | UUCGGGGAACGCCAGCGUGCGCUGGCCGAUCCCGAGGCGAGGUGCCGUAGCGGGCACCCGUCGUAAC | splicing |
| | GCGCGGUAGGGCGUCGGUCCCCCCUCCCACGGCGGAGGGGAGGCUUAAGGUACGUGCUAAACCCCCA | intron |
| | GCGAUGGGGCCUGUAGGAAAAGCCCUGGUACGGCGAAGCCUACGGGGACCGCCCGAUGGCGGUCGGA | fragment |
| | UGCCGGCGGGCCACGGAGCCCCCGGCGUCGAUUG | derived from |
| | | Scytalidium |
| | | dimidiatum |
| | | GROUP I |
| | | INTRON-PIE-2 |
| 38 | UGACGACAACAGGCCUGUGACAGCGGGGCUUCGAACUUUCCGUGUUUUUUUUUCUUCCGCUAGUCGA | 5′ self- |
| | UCAUCCGUGACGGCCGGGCAA | splicing |
| | | intron |
| | | fragment |
| | | derived from |
| | | Scytalidium |
| | | dimidiatum |
| | | GROUP I |
| | | INTRON-PIE-2 |
| 39 | GCGCUGGCCGAUCCCGAGGCGAGGUGCCGUAGCGGGCACCCGUCGUAACGCGCGGUAGGGCGUCGGU | 3′ self- |
| | CCCCCCUCCCACGGCGGAGGGGAGGCUUAAGGUACGUGCUAAACCCCCAGCGAUGGGGCCUGUAGGA | splicing |
| | AAAGCCCUGGUACGGCGAAGCCUACGGGGACCGCCCGAUGGCGGUCGGAUGCCGGCGGGCCACGGAG | intron |
| | CCCCCGGCGUCGAUUG | fragment |
| | | derived from |
| | | Scytalidium |
| | | dimidiatum |
| | | GROUP I |
| | | INTRON-PIE-5 |
| 40 | UGACGACAACAGGCCUGUGACAGCGGGGCUUCGAACUUUCCGUGUUUUUUUUUCUUCCGCUAGUCGA | 5′ self- |
| | UCAUCCGUGACGGCCGGGCAAGCGCCCGAGUACCAGGACCGUCGGGCCCCCGAGAAGGGGGGGCCUA | splicing |
| | GGAUGCGGCAAGACGACCCGGUUCGGGGAACGCCAGCGU | intron |
| | | fragment |
| | | derived from |
| | | Scytalidium |
| | | dimidiatum |
| | | GROUP I |
| | | INTRON-PIE-5 |
| 41 | GCGGGCACCCGUCGUAACGCGCGGUAGGGCGUCGGUCCCCCCUCCCACGGCGGAGGGGAGGCUUAAG | 3′ self- |
| | GUACGUGCUAAACCCCCAGCGAUGGGGCCUGUAGGAAAAGCCCUGGUACGGCGAAGCCUACGGGGAC | splicing |
| | CGCCCGAUGGCGGUCGGAUGCCGGCGGGCCACGGAGCCCCCGGCGUCGAUUG | intron |
| | | fragment |
| | | derived from |
| | | Scytalidium |
| | | dimidiatum |
| | | GROUP I |
| | | INTRON-PIE-6 |
| 42 | UGACGACAACAGGCCUGUGACAGCGGGGCUUCGAACUUUCCGUGUUUUUUUUUCUUCCGCUAGUCGA | 5′ self- |
| | UCAUCCGUGACGGCCGGGCAAGCGCCCGAGUACCAGGACCGUCGGGCCCCCGAGAAGGGGGGGCCUA | splicing |
| | GGAUGCGGCAAGACGACCCGGUUCGGGGAACGCCAGCGUGCGCUGGCCGAUCCCGAGGCGAGGUGCC | intron |
| | GUA | fragment |
| | | derived from |
| | | Scytalidium |
| | | dimidiatum |
| | | GROUP I |
| | | INTRON-PIE-6 |
| 43 | GGCGGAGGGGAGGCUUAAGGUACGUGCUAAACCCCCAGCGAUGGGGCCUGUAGGAAAAGCCCUGGUA | 3′ self- |
| | CGGCGAAGCCUACGGGGACCGCCCGAUGGCGGUCGGAUGCCGGCGGGCCACGGAGCCCCCGGCGUCG | splicing |
| | AUUG | intron |
| | | fragment |
| | | derived from |
| | | Scytalidium |
| | | dimidiatum |
| | | GROUP I |
| | | INTRON-PIE-8 |
| 44 | UGACGACAACAGGCCUGUGACAGCGGGGCUUCGAACUUUCCGUGUUUUUUUUUCUUCCGCUAGUCGA | 5′ self- |
| | UCAUCCGUGACGGCCGGGCAAGCGCCCGAGUACCAGGACCGUCGGGCCCCCGAGAAGGGGGGGCCUA | splicing |
| | GGAUGCGGCAAGACGACCCGGUUCGGGGAACGCCAGCGUGCGCUGGCCGAUCCCGAGGCGAGGUGCC | intron |
| | GUAGCGGGCACCCGUCGUAACGCGCGGUAGGGCGUCGGUCCCCCCUCCCAC | fragment |
| | | derived from |
| | | Scytalidium |
| | | dimidiatum |
| | | GROUP I |
| | | INTRON-PIE-8 |
| 45 | GGAGCCCCCGGCGUCGAUUG | 3′ self- |
| | | splicing |
| | | intron |
| | | fragment |
| | | derived from |
| | | Scytalidium |
| | | dimidiatum |
| | | GROUP I |
| | | INTRON-PIE-9 |
| 46 | UGACGACAACAGGCCUGUGACAGCGGGGCUUCGAACUUUCCGUGUUUUUUUUUCUUCCGCUAGUCGA | 5′ self- |
| | UCAUCCGUGACGGCCGGGCAAGCGCCCGAGUACCAGGACCGUCGGGCCCCCGAGAAGGGGGGGCCUA | splicing |
| | GGAUGCGGCAAGACGACCCGGUUCGGGGAACGCCAGCGUGCGCUGGCCGAUCCCGAGGCGAGGUGCC | intron |
| | GUAGCGGGCACCCGUCGUAACGCGCGGUAGGGCGUCGGUCCCCCCUCCCACGGCGGAGGGGAGGCUU | fragment |
| | AAGGUACGUGCUAAACCCCCAGCGAUGGGGCCUGUAGGAAAAGCCCUGGUACGGCGAAGCCUACGGG | derived from |
| | GACCGCCCGAUGGCGGUCGGAUGCCGGCGGGCCAC | Scytalidium |
| | | dimidiatum |
| | | GROUP I |
| | | INTRON-PIE-9 |
| 47 | AAUGGAGAAGGUGUAGAGACUGGAAGGCAGGUACCCUAACAGUAAAGCUGAGGGUAAAGGGACAGUC | 3′ self- |
| | CAGACCACAAACUGGUUAAUCCAGGCAGUGAAAACUGUAGAUGGUAAG | splicing |
| | | intron |
| | | fragment |
| | | derived from |
| | | Scytonema- |
| | | hofmani tRNA |
| | | fMet gene |
| | | GROUP I |
| | | INTRON-PIE-6 |
| 48 | CAACGAGUAAGAUUGACUAAACGCUUAAAAGUUAUUCUUACUAUGGGCGGUACGUAAAGAAACUUAC | 5′ self- |
| | GUAUGUUUACCUGUCAAACUCGGGGAAGCCAAUAGCGCGGUAAUCCCGAACCAAGCUCCAGA | splicing |
| | | intron |
| | | fragment |
| | | derived from |
| | | Scytonema- |
| | | hofmani tRNA |
| | | fMet gene |
| | | GROUP I |
| | | INTRON-PIE-6 |
| 49 | AACUUACGUAUGUUUACCUGUCAAACUCGGGGAAGCCAAUAGCGCGGUAAUCCCGAACCAAGCUCCA | 3′ self- |
| | GAAAUGGAGAAGGUGUAGAGACUGGAAGGCAGGUACCCUAACAGUAAAGCUGAGGGUAAAGGGACAG | splicing |
| | UCCAGACCACAAACUGGUUAAUCCAGGCAGUGAAAACUGUAGAUGGUAAG | intron |
| | | fragment |
| | | derived from |
| | | Scytonema- |
| | | hofmani tRNA |
| | | fMet gene |
| | | GROUP I |
| | | INTRON-PIE-2 |
| 50 | CAACGAGUAAGAUUGACUAAACGCUUAAAAGUUAUUCUUACUAUGGGCGGUACGUAAAGA | 5′ self- |
| | | splicing |
| | | intron |
| | | fragment |
| | | derived from |
| | | Scytonema- |
| | | hofmani tRNA |
| | | fMet gene |
| | | GROUP I |
| | | INTRON-PIE-2 |
| 51 | AUCCCGAACCAAGCUCCAGAAAUGGAGAAGGUGUAGAGACUGGAAGGCAGGUACCCUAACAGUAAAG | 3′ self- |
| | CUGAGGGUAAAGGGACAGUCCAGACCACAAACUGGUUAAUCCAGGCAGUGAAAACUGUAGAUGGUAA | splicing |
| | G | intron |
| | | fragment |
| | | derived from |
| | | Scytonema- |
| | | hofmani tRNA |
| | | fMet gene |
| | | GROUP I |
| | | INTRON-PIE- |
| | | 4-5 |
| 52 | CAACGAGUAAGAUUGACUAAACGCUUAAAAGUUAUUCUUACUAUGGGCGGUACGUAAAGAAACUUAC | 5′ self- |
| | GUAUGUUUACCUGUCAAACUCGGGGAAGCCAAUAGCGCGGUA | splicing |
| | | intron |
| | | fragment |
| | | derived from |
| | | Scytonema- |
| | | hofmani tRNA |
| | | fMet gene |
| | | GROUP I |
| | | INTRON-PIE- |
| | | 4-5 |
| 53 | AAUCCAGGCAGUGAAAACUGUAGAUGGUAAG | 3′ self- |
| | | splicing |
| | | intron |
| | | fragment |
| | | derived from |
| | | Scytonema- |
| | | hofmani tRNA |
| | | fMet gene |
| | | GROUP I |
| | | INTRON-PIE-9 |
| 54 | CAACGAGUAAGAUUGACUAAACGCUUAAAAGUUAUUCUUACUAUGGGCGGUACGUAAAGAAACUUAC | 5′ self- |
| | GUAUGUUUACCUGUCAAACUCGGGGAAGCCAAUAGCGCGGUAAUCCCGAACCAAGCUCCAGAAAUGG | splicing |
| | AGAAGGUGUAGAGACUGGAAGGCAGGUACCCUAACAGUAAAGCUGAGGGUAAAGGGACAGUCCAGAC | intron |
| | CACAAACUGGUU | fragment |
| | | derived from |
| | | Scytonema- |
| | | hofmani tRNA |
| | | fMet gene |
| | | GROUP I |
| | | INTRON-PIE-9 |
| 55 | AAUCCGGUGAAGGUGUAGAGACUGGAUGGGCAGCACCUAAAGGCCGCAAGGUCCACGGUGAAGGGAC | 3′ self- |
| | AGUCCAGACCACGAACGCCCUUAUCCGAUUGAGAUGGGCGGCGGCGAAAGCCGAAGCGGUAUG | splicing |
| | | intron |
| | | fragment |
| | | derived from |
| | | Agrobacterium |
| | | tumefaciens |
| | | GROUP I |
| | | INTRON-PIE-6 |
| 56 | AAGGUAAAUUGGGGGUUGCGCCCGGAAACGACGCAAUCGAUCUGCUCAAAGUCGGGGAAAGCUUCGC | 5′ self- |
| | UGGUCUUCCAGUAUGCCAAUCCCGAGCCAAGCUCCGGAGA | splicing |
| | | intron |
| | | fragment |
| | | derived from |
| | | Agrobacterium |
| | | tumefaciens |
| | | GROUP I |
| | | INTRON-PIE-6 |
| 57 | AAAAUCCGUUGA | 3′ exon of |
| | | GROUP I |
| | | INTRON from |
| | | Anabaena |
| | | pre-tRNA-Leu |
| | | gene |
| 58 | AGACGCUACGGACUU | 5′ exon of |
| | | GROUP I |
| | | INTRON from |
| | | Anabaena |
| | | pre-tRNA-Leu |
| | | gene |
| 59 | AAAACCCCCCCA | 3′ exon of |
| | | GROUP I |
| | | INTRON from |
| | | Anabaena |
| | | pre-tRNA-Leu |
| | | gene |
| 60 | AGACGGGGGGGGCUU | 5′ exon of |
| | | GROUP I |
| | | INTRON from |
| | | Anabaena |
| | | pre-tRNA-Leu |
| | | gene |
| 61 | AAUCCGUUGGUGCUG | 3′ exon of |
| | | GROUP I |
| | | INTRON from |
| | | Azoarcus |
| | | olearius |
| | | BH72 |
| 62 | GAGCAGCGGACUCAU | 5′ exon of |
| | | GROUP I |
| | | INTRON from |
| | | Azoarcus |
| | | olearius |
| | | BH72 |
| 63 | AACUUUUAGUUAU | 3′ exon of |
| | | GROUP I |
| | | INTRON from |
| | | Staphylococcus |
| | | phage |
| | | Twort |
| 64 | ACUACUGAAAGCAU | 5′ exon of |
| | | GROUP I |
| | | INTRON from |
| | | Staphylococcus |
| | | phage |
| | | Twort |
| 65 | CUGUUA | 3′ exon of |
| | | GROUP I |
| | | INTRON from |
| | | Scytalidium |
| | | dimidiatum |
| 66 | UAACAGGU | 5′ exon of |
| | | GROUP I |
| | | INTRON from |
| | | Scytalidium |
| | | dimidiatum |
| 67 | CAUAACCC | 3′ exon of |
| | | GROUP I |
| | | INTRON from |
| | | Scytonema- |
| | | hofmani tRNA |
| | | fMet gene |
| 68 | GGGCU | 5′ exon of |
| | | GROUP I |
| | | INTRON from |
| | | Scytonema- |
| | | hofmani tRNA |
| | | fMet gene |
| 69 | AAUCCUGGGG | 3′ exon of |
| | | GROUP I |
| | | INTRON from |
| | | Agrobacterium |
| | | tumefaciens |
| 70 | CAGGAUUCCU | 5′ exon of |
| | | GROUP I |
| | | INTRON from |
| | | Agrobacterium |
| | | tumefaciens |
| 71 | CCGUCGAUUGUCCACUGGUC | 5′ homology |
| | | arm-original |
| | | (20) |
| 72 | GACCAGUGGACAAUCGACGG | 3′ homology |
| | | arm-original |
| | | (20) |
| 73 | CCGUCGAUUGUCCACUGGUCCCGUCGAUUGUCCACUGGUC | 5′ homology |
| | | arm- |
| | | 2 × original |
| | | (40) |
| 74 | GACCAGUGGACAAUCGACGGGACCAGUGGACAAUCGACGG | 3′ homology |
| | | arm- |
| | | 2 × original |
| | | (40) |
| 75 | GGACACCCCCUGACCCGGUCUCAGAAAGCCUGAAAGCAGAAUUAGUCAUUAGAAGGGUGGUUGGCUU | 5′ homology |
| | GGUCGGCAUAGACUUUGAGCAGAAAGAGGUUGAAAAUGUUGAGCCUGAUUUCUCUUAGGCCCCUCUG | arm-HA1 |
| | CAGUGUCUGUUGUGGAGGCCAGAUACGUAACUGCUUCCGCUUUUUUUGGUCUCAUUCAAGGUGAGCA | (300) |
| | AAUCCCCUUCAUGUUUCUCACCAGACAAUGCAGCUGAUGAGGUUCCAGCUUUGCAAAUGUAGUCAUC | |
| | CAUGAGGACUGUCUUCCUGAGAUUUCAUCAGG | |
| 76 | CCUGAUGAAAUCUCAGGAAGACAGUCCUCAUGGAUGACUACAUUUGCAAAGCUGGAACCUCAUCAGC | 3′ homology |
| | UGCAUUGUCUGGUGAGAAACAUGAAGGGGAUUUGCUCACCUUGAAUGAGACCAAAAAAAGCGGAAGC | arm-HA1 |
| | AGUUACGUAUCUGGCCUCCACAACAGACACUGCAGAGGGGCCUAAGAGAAAUCAGGCUCAACAUUUU | (300) |
| | CAACCUCUUUCUGCUCAAAGUCUAUGCCGACCAAGCCAACCACCCUUCUAAUGACUAAUUCUGCUUU | |
| | CAGGCUUUCUGAGACCGGGUCAGGGGGUGUCC | |
| 77 | GGACACCCCCUGACCCGGUCUCAGAAAGCCUGAAAGCAGAAUUAGUCAUUAGAAGGGUGGUUGGCUU | 5′ homology |
| | GGUCGGCAUAGACUUUGAGCAGAAAGAGGUUGA | arm-HA2 |
| | | (100) |
| 78 | UCAACCUCUUUCUGCUCAAAGUCUAUGCCGACCAAGCCAACCACCCUUCUAAUGACUAAUUCUGCUU | 3′ homology |
| | UCAGGCUUUCUGAGACCGGGUCAGGGGGUGUCC | arm-HA2 |
| | | (100) |
| 79 | AAAGAAGACAGUCUCGCUCUAUCGCCCAGGCUGGAGUGUAGAAUGCAGUGCUGUGAUCUUGGCUCAC | 5′ homology |
| | UGCAACCUCUACCUCCCAGGCUCAAGCGAUUCUUCUGCCUCAGCCUCCCGAGUAGCUAGGAUUACAG | arm-HA3 |
| | GCGCGCGCCACCACACCUAGCUUAUUUUUGUAUUUUCAGUAGAGAUGGGGUUUCACCAUGUUGACCA | (285) |
| | GGCUGGUCUCAAACUCCUGAGCUUAAGUGACGUGCCCGCCUCAGCCUCCCAAAGUGCUUGGAUUAUA | |
| | GGUGUGAGCCACUGCAC | |
| 80 | CAUCUCUACUGAAAAAUACAAAAAAUUAGCCGGGCGUAGUGGCAUGAGCCUGUAGUCCCAGCUACUU | 3′ homology |
| | GGGAGGCUGAGGCAGGAGAAUGGCGUGAACUCGGGAGGCAGAGCUUGCAGUGAGCCGAGAUCACACC | arm-HA3 |
| | ACUGCACUCCAGCCUGGGCGACAGAGCGAGACUCCGUCUC | (174) |
| 81 | AGAGAGUCUCGCUCUUCGCCCAGGCUGGAGUGAGUGGUGUGAUCUGGCUCACUGCAACUCUCCUCCC | 5′ homology |
| | GUCAGCAUUCUCUGCCUCAGCCUCCCAGUAGCUGGAUACAGGCCGCCACACCCGCUAUUUUUGUAUU | arm-HA4 |
| | UUCAGUAGAGAUG | (147) |
| 82 | CAUCUCUACUGAAAAUACAAAAAUAGCGGGUGUGGCGGCCUGUAUCCAGCUACUGGGAGGCUGAGGC | 3′ homology |
| | AGAGAAUGCUGACGGGAGGAGAGUUGCAGUGAGCCAGAUCACACCACUCACUCCAGCCUGGGCGAAG | arm-HA4 |
| | AGCGAGACUCUCU | (147) |
| 83 | GGCCCCUCUGCAGUGUCUGUUGUGGAGGCCAGAUACGUAACUGCUUCCGC | 5′ homology |
| | | arm-HA5 (50) |
| 84 | GCGGAAGCAGUUACGUAUCUGGCCUCCACAACAGACACUGCAGAGGGGCC | 3′ homology |
| | | arm-HA5 (50) |
| 85 | UAGACUUUGAGCAGAAAGAGGUUGAAAAUGUUGAGCCUGAUUUCUCUUAG | 5′ homology |
| | | arm-HA6 (50) |
| 86 | CUAAGAGAAAUCAGGCUCAACAUUUUCAACCUCUUUCUGCUCAAAGUCUA | 3′ homology |
| | | arm-HA6 (50) |
| 87 | AUGAGGUUCCAGCUUUGCAAAUGUAGUCAUCCAUGAGGACUGUCUUCCUG | 5′ homology |
| | | arm-HA7 (50) |
| 88 | CAGGAAGACAGUCCUCAUGGAUGACUACAUUUGCAAAGCUGGAACCUCAU | 3′ homology |
| | | arm-HA7 (50) |
| 89 | GGACACCCCCUGACCCGGUCUCAGA | 5′ homology |
| | | arm-HA8 (25) |
| 90 | UCUGAGACCGGGUCAGGGGGUGUCC | 3′ homology |
| | | arm-HA8 |
| | | (25) |
| 91 | GAAAGCAGAAUUAGUCAUUAGAAGG | 5′ homology |
| | | arm-HA9 (25) |
| 92 | CCUUCUAAUGACUAAUUCUGCUUUC | 3′ homology |
| | | arm-HA9 (25) |
| 93 | CGGCAUAGACUUUGAGCAGAAAGAG | 5′ homology |
| | | arm-HA10 |
| | | (25) |
| 94 | CUCUUUCUGCUCAAAGUCUAUGCCG | 3′ homology |
| | | arm-HA10 |
| | | (25) |
| 95 | CUU | Nucleotide |
| | | motif of |
| | | ribozyme |
| | | recognition |
| | | site I of |
| | | Aaz.b.trnL, |
| | | Asp- |
| | | 2.b.trnL, |
| | | Cpa.y.trnL- |
| | | 1, |
| | | Dai.c.trnL- |
| | | 1, |
| | | Dai.c.trnL- |
| | | 2, |
| | | Dai.c.trnL- |
| | | 3, |
| | | Ddu.c.trnL- |
| | | 1, |
| | | Ddu.c.trnL- |
| | | 2, |
| | | Dla.c.trnL, |
| | | Dto.c.trnL- |
| | | 1, |
| | | Dto.c.trnL- |
| | | 2, Dto.c.trnL- |
| | | 3, |
| | | Fan.c.trnL, |
| | | Zma.c.trnL, |
| | | Vfa.c.trnL, |
| | | Sel.b.trnL- |
| | | 2, |
| | | Sbi.c.trnL, |
| | | Pth.c.trnL, |
| | | Osa.c.trnL, |
| | | Omi.c.trnL, |
| | | Nta.c.trnL, |
| | | Mpo.c.trnL, |
| | | Mae.b.trnL- |
| | | 2, |
| | | Dto.c.trnL- |
| | | 4, |
| 96 | CCU | Nucleotide |
| | | motif of |
| | | ribozyme |
| | | recognition |
| | | site I of |
| | | Aha.b.trnR, |
| | | Atu.b.trnR, |
| 97 | CAU | Nucleotide |
| | | motif of |
| | | ribozyme |
| | | recognition |
| | | site I of |
| | | Asp.b.trnI, |
| | | Twort.v.ORF1 |
| | | 42,2 |
| 98 | UU | Nucleotide |
| | | motif of |
| | | ribozyme |
| | | recognition |
| | | site I of |
| | | Athu.c.trnL- |
| | | 1 |
| 99 | CU | Nucleotide |
| | | motif of |
| | | ribozyme |
| | | recognition |
| | | site I of |
| | | Dsp.b.trnM, |
| | | Sho.b.trnM |
| 100 | GGCU | Nucleotide |
| | | motif of |
| | | ribozyme |
| | | recognition |
| | | site I of |
| | | Gvi.b.trnM, |
| | | Ssp.b.trnM |
| 101 | GCU | Nucleotide |
| | | motif of |
| | | ribozyme |
| | | recognition |
| | | site I of |
| | | Pan.m.COX1, |
| | | 16 |
| 102 | CGUCAU | Nucleotide |
| | | motif of |
| | | ribozyme |
| | | recognition |
| | | site I of |
| | | Ppo.L1949- |
| | | 1.pdf |
| 103 | CGUCAU | Nucleotide |
| | | motif of |
| | | ribozyme |
| | | recognition |
| | | site I of |
| | | Ppo.L1949- |
| | | 2.pdf |
| 104 | GGGAU | Nucleotide |
| | | motif of |
| | | ribozyme |
| | | recognition |
| | | site I of |
| | | Ppo.L2449- |
| | | 1.pdf, |
| | | Ppo.L2449- |
| | | 2 .pdf |
| 105 | GGAU | Nucleotide |
| | | motif of |
| | | ribozyme |
| | | recognition |
| | | site I of |
| | | Ban.b.nrdE |
| 106 | GGAAAU | Nucleotide |
| | | motif of |
| | | ribozyme |
| | | recognition |
| | | site I of |
| | | Ban.b.recA |
| 107 | UUGGAGCC | Nucleotide |
| | | motif of |
| | | ribozyme |
| | | recognition |
| | | site I of |
| | | Beta- |
| | | 22.v.td, 1 |
| 108 | UGGAGGU | Nucleotide |
| | | motif of |
| | | ribozyme |
| | | recognition |
| | | site I of |
| | | Ceu.c.psbC, 1 |
| 109 | AAGGU | Nucleotide |
| | | motif of |
| | | ribozyme |
| | | recognition |
| | | site I of |
| | | Ceu.mL1931.p |
| | | df |
| 110 | GGAGGU | Nucleotide |
| | | motif of |
| | | ribozyme |
| | | recognition |
| | | site I of |
| | | Cre.c.psbA, 3 |
| 111 | AGUGUGGUU | Nucleotide |
| | | motif of |
| | | ribozyme |
| | | recognition |
| | | site I of |
| | | LL- |
| | | H.v.terL, 1 |
| 112 | GCCU | Nucleotide |
| | | motif of |
| | | ribozyme |
| | | recognition |
| | | site I of |
| | | Phi- |
| | | E.v.DP, 1, |
| | | SP82.v.DP, 1, |
| | | SPO1.v.DP, 1 |
| 113 | GCGU | Nucleotide |
| | | motif of |
| | | ribozyme |
| | | recognition |
| | | site I of |
| | | RB3.v.nrdB, 1, |
| | | T4 .v .nrdB, 1 |
| 114 | UAUGAGU | Nucleotide |
| | | motif of |
| | | ribozyme |
| | | recognition |
| | | site I of |
| | | T4.v.nrdD, 1 |
| 115 | GGGGU | Nucleotide |
| | | motif of |
| | | ribozyme |
| | | recognition |
| | | site I of |
| | | T4.v.td, 1 |
| 116 | GGU | Nucleotide |
| | | motif of |
| | | ribozyme |
| | | recognition |
| | | site I of |
| | | Sdi.S1199-1 |
| 117 | AUGUAUAGAAUGCAGCUGCUGUCUUGCAUCGCCCUGAGCCUGGCCCUGGUCACAAACAGCGCUCCUA | IL2-N88D |
| | CAAGCUCUAGCACCAAGAAAACCCAGCUGCAGCUCGAGCACCUGCUGCUGGACCUGCAGAUGAUCCU | |
| | GAACGGCAUUAACAACUACAAGAACCCAAAGCUGACCAGAAUGCUGACAUUUAAGUUCUACAUGCCU | |
| | AAGAAGGCCACAGAGCUGAAGCACCUGCAAUGUCUGGAAGAGGAACUGAAGCCCCUGGAAGAGGUGC | |
| | UUAAUCUGGCUCAGAGCAAGAACUUCCACCUGAGACCUCGGGACCUGAUCAGCGACAUCAACGUGAU | |
| | CGUGCUGGAACUGAAAGGCAGCGAGACAACCUUCAUGUGCGAGUACGCCGAUGAGACCGCCACCAUC | |
| | GUGGAGUUCCUGAAUCGGUGGAUCACCUUCUGCCAGUCCAUCAUCUCCACCCUGACC | |
| 118 | AUGGCCAGCGAGUUCAAGAAGAAGCUCUUCUGGAGGGCAGUGGUGGCCGAGUUCCUGGCCACGACCC | AQP1 |
| | UCUUUGUCUUCAUCAGCAUCGGUUCUGCCCUGGGCUUCAAAUACCCGGUGGGGAACAACCAGACGGC | |
| | GGUCCAGGACAACGUGAAGGUGUCGCUGGCCUUCGGGCUGAGCAUCGCCACGCUGGCGCAGAGUGUG | |
| | GGCCACAUCAGCGGCGCCCACCUCAACCCGGCUGUCACACUGGGGCUGCUGCUCAGCUGCCAGAUCA | |
| | GCAUCUUCCGUGCCCUCAUGUACAUCAUCGCCCAGUGCGUGGGGGCCAUCGUCGCCACCGCCAUCCU | |
| | CUCAGGCAUCACCUCCUCCCUGACUGGGAACUCGCUUGGCCGCAAUGACCUGGCUGAUGGUGUGAAC | |
| | UCGGGCCAGGGCCUGGGCAUCGAGAUCAUCGGGACCCUCCAGCUGGUGCUAUGCGUGCUGGCUACUA | |
| | CCGACCGGAGGCGCCGUGACCUUGGUGGCUCAGCCCCCCUUGCCAUCGGCCUCUCUGUAGCCCUUGG | |
| | ACACCUCCUGGCUAUUGACUACACUGGCUGUGGGAUUAACCCUGCUCGGUCCUUUGGCUCCGCGGUG | |
| | AUCACACACAACUUCAGCAACCACUGGAUUUUCUGGGUGGGGCCAUUCAUCGGGGGAGCCCUGGCUG | |
| | UACUCAUCUACGACUUCAUCCUGGCCCCACGCAGCAGUGACCUCACAGACCGCGUGAAGGUGUGGAC | |
| | CAGCGGCCAGGUGGAGGAGUAUGACCUGGAUGCCGACGACAUCAACUCCAGGGUGGAGAUGAAGCCC | |
| | AAA | |
| 119 | GGUGGAACGGCACAUGUGUGAUGGGGACAUUGUUAUCUUCAACCGGCAGCCAACUCUGCACAAAAUG | POLR2A |
| | UCCAUGAUGGGGCAUCGGGUCCGCAUUCUCCCAUGGUCUACCUUUCGCUUGAAUCUUAGUGUGACAA | |
| | CUCCGUACAAUGCAGACUUUGACGGGGAUGAGAUGAACUUGCACCUGCCACAGUCUCUGGAGACGCG | |
| | AGCAGAGAUCCAGGAGCUGGCCAUGGUUCCUCGCAUGAUUGUCACCCCCCAGAGCAAUCGGCCUGUC | |
| | AUGGGUAUUGUGCAGGACACACUCACAGCAGUGCGCAAAUUCACCAAGAGAGACGUCUUCCUGGAGC | |
| | GG | |
| 120 | GUAUAAGAGACAGGUGUUUGCCUUGUCUUCGGACUGGCAUCUUGGGACCAACCCCCCUUUUCCCCAG | CrohivirusB |
| | CCAUGGGUUAAAUGGCAAUAAAGGACGUAACAACUUUGUAACCAUUAAGCUUUGUAAUUUUGUAACC | |
| | ACUAAGCUUUGUGCACAUAAUGUAACCAUCAAGCUUGUUAGUCCCAGCAGGAGGUUUGCAUGCUUGU | |
| | AGCCGAAAUGGGGCUCGACCCCCCAUAGUAGGAUACUUGAUUUUGCAUUCCAUUGUGGACCUGCAAA | |
| | CUCUACACAUAGAGGCUUUGUCUUGCAUCUAAACACCUGAGUACAGUGUGUACCUAGACCCUAUAGU | |
| | ACGGGAGGACCGUUUGUUUCCUCAAUAACCCUACAUAAUAGGCUAGGUGGGCAUGCCCAAUUUGCAA | |
| | GAUCCCAGACUGGGGGUCGGUCUGGGCAGGGUUAGAUCCCUGUUAGCUACUGCCUGAUAGGGUGGUG | |
| | CUCAACCAUGUGUAGUUUAAAUUGAGCUGUUCAUAUACC | |
| 121 | UUAAAACAGCCUGUGGGUUGAUCCCACCCACAGGCCCAUUGGGCGCUAGCACUCUGGUAUCACGGUA | CVB3 |
| | CCUUUGUGCGCCUGUUUUAUACCCCCUCCCCCAACUGUAACUUAGAAGUAACACACACCGAUCAACA | |
| | GUCAGCGUGGCACACCAGCCACGUUUUGAUCAAGCACUUCUGUUACCCCGGACUGAGUAUCAAUAGA | |
| | CUGCUCACGCGGUUGAAGGAGAAAGCGUUCGUUAUCCGGCCAACUACUUCGAAAAACCUAGUAACAC | |
| | CGUGGAAGUUGCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCAUGGGACGCUC | |
| | UAAUACAGACAUGGUGCGAAGAGUCUAUUGAGCUAGUUGGUAGUCCUCCGGCCCCUGAAUGCGGCUA | |
| | AUCCUAACUGCGGAGCACACACCCUCAAGCCAGAGGGCAGUGUGUCGUAACGGGCAACUCUGCAGCG | |
| | GAACCGACUACUUUGGGUGUCCGUGUUUCAUUUUAUUCCUAUACUGGCUGCUUAUGGUGACAAUUGA | |
| | GAGAUCGUUACCAUAUAGCUAUUGGAUUGGCCAUCCGGUGACUAAUAGAGCUAUUAUAUAUCCCUUU | |
| | GUUGGGUUUAUACCACUUAGCUUGAAAGAGGUUAAAACAUUACAAUUCAUUGUUAAGUUGAAUACAG | |
| | CAAA | |
| 122 | ACAUGGGGGGUCUGCGGACGGCUUCGGCCCACCCGCGACAAGAAUGCCGUCAUCUGUCCUCAUUACC | SalivirusFHB |
| | CGUAUUCCUUCCCUUCCCCCGCAACCACCACGCUUACUCGCGCACGUGUUGAGUGGCACGUGCGUUG | |
| | UCCAAACAGCUACACCCACACCCUUCGGGGGGGGUUUGUCCCGCCCUCGGGUUCCUCGCGGAACCCC | |
| | CCCCUCCCUCUCUCUCUUUCUAUCCGCCCUCACUUCCCAUAACUACAGUGCUUUGGUAGGUGAGCAC | |
| | CCUGACCCCCCGCGGAAGCUGCUAACGUGGCAACUGUGGGGAUCCAGGCAGGUUAUCAAAGGCACCC | |
| | GGUCUUUCCGCCUUCAGGAGUAUCUCUGCCGGUGAAUUCCGGUAGGGCUCUGCUUGGUGCCAACCUC | |
| | CCCCAAAUGCGCGCUGCGGGAGUGCUCUUCCCCAACUCAUCUUAGUAACCUCUCAUGUGUGUGCUUG | |
| | GUCAGCAUAUCUGAGGCGACGUUCCGCUGUCCCAGACCAGUCCAGCAAUGGACGGGCCAGUGUGCGU | |
| | AGUCGCUUUCCGGUUUUCCGGCGCAUGUUUGGCGAAACGCUGAGGUAAGGUUGGUGUGCCCAACGCC | |
| | CGUAAUUUGGUGAUACCUCAAGACCACCCAGGAAUGCCAGGGAGGUACCCCACUUCGGUGGGAUCUG | |
| | ACCCUGGGCUAAUUGUCUACGGUGGUUCUUCUUGCUUCCACUUCUCUUUUUUCUGGCAUG | |
| 123 | CCCACAGCAAGAAUGCCAUCAUCUGUCCUCACCCCCAAUUUUCCCUUUUCUUCCCCUGCAACCAUUA | 18-675 |
| | CGCUUACUCGCAUGUGCAUUGAGUGGUGCAUGUGUUGAACAAACAGCUACACUCACAUGGGGGCGGG | |
| | UUUUCCCGCCCUACGGCCUCUCGCGAGGCCCACCCCUUCCCUCCCCUUAUAACUACAGUGCUUUGGU | |
| | AGGUAAGCAUCCUGAUCCCCCGCGGAAGCUGCUCACGUGGCAACUGUGGGGACCCAGACAGGUUAUC | |
| | AAAGGCACCCGGUCUUUCCGCCUUCAGGAGUAUCCCUACUAGUGAAUUCUAGCGGGGCUCUGCUUGG | |
| | UGCCAACCUCCCCCAAAUGCGCGCUGCGGGAGUGCUCUUCCCCAACUCACCCUAGUAUCCUCUCAUG | |
| | UGUGUGCUUGGUCAGCAUAUCUGAGACGAUGUUCCGCUGUCCCAGACCAGUCCAGUAAUGGACGGGC | |
| | CAGUGCGUGUAGUCGUCUUCCGGCUUGUCCGGGGCAUGUUUGGUGAACCGGUGGGGUAAGGUUGGUG | |
| | UGCCCAACGCCCGUACUUUGGUGACACCUCAAGACCACCCAGGAAUGCCAGGGAGGUACCCCACCUC | |
| | ACGGUGGGAUCUGACCCUGGGCUAAUUGUCUACGGUGGUUCUUCUUGCUUCCACUUCUUUCUUCUGU | |
| | UCACG | |
| 124 | UUUGAAAAGGGGGUGGGGGGGCCUCGGCCCCCUCACCCUCUUUUCCGGUGGUCUGGUCCCGGACCAC | Aichivirus |
| | CGUUACUCCAUUCAGCUUCUUCGGAACCUGUUCGGAGGAAUUAAACGGGCACCCAUACUCCCCCCAC | |
| | CCCCCUUUUGUAACUAAGUAUGUGUGCUCGUGAUCUUGACUCCCACGGAACGGACCGAUCCGUUGGU | |
| | GAACAAACAGCUAGGUCCACAUCCUCCCUUCCCCUGGGAGGGCCCCCGCCCUCCCACAUCCUCCCCC | |
| | CAGCCUGACGUAUCACAGGCUGUGUGAAGCCCCCGCGAAAGCUGCUCACGUGGCAAUUGUGGGUCCC | |
| | CCCUUCAUCAAGACACCAGGUCUUUCCUCCUUAAGGCUAGCCCCGGCGUGUGAAUUCACGUUGGGCA | |
| | ACUAGUGGUGUCACUGUGCGCUCCCAAUCUCGGCCGCGGAGUGCUGUUCCCCAAGCCAAACCCCUGG | |
| | CCCUUCACUAUGUGCCUGGCAAGCAUAUCUGAGAAGGUGUUCCGCUGUGGCUGCCAACCUGGUGACA | |
| | GGUGCCCCAGUGUGCGUAACCUUCUUCCGUCUCCGGACGGUAGUGAUUGGUUAAGAUUUGGUGUAAG | |
| | GUUCAUGUGCCAACGCCCUGUGCGGGAUGAAACCUCUACUGCCCUAGGAAUGCCAGGCAGGUACCCC | |
| | ACCUCCGGGUGGGAUCUGAGCCUGGGCUAAUUGUCUACGGGUAGUUUCAUUUCCAAUCCUUUUAUGU | |
| | CGGAGUC | |
| 125 | UUAAAACAGCCUGUGGGUUGCACCCACCCACAGGGCCCACUGGGCGCUAGCACUCUGGUAUCACGAU | CAV2 |
| | ACCUUUGUGCGCCUGUUUUAUAUCCCCACCCCGAGUAAACGUUAGAAGUUACGCAACCCCGAUCAAU | |
| | AGUAGGUGUAGCACUCCAGCUGCAUCGAGAUCAAGCACUUCUGUCUCCCCGGACCGAGUAUCAAUAG | |
| | ACUGCUAACGCGGUUGAAGGAGAAAACGUUCGUUACCCGGCCAAUUACUUCGAGAAGCCCAGUAGUG | |
| | CCGUGAAAGUUGCGGAGUGUUUCGCUCAGCACUUCCCCCGUGUAGAUCAGGCUGAUGAGUCACCGCG | |
| | AUCCCCACAGGUGACUGUGGCGGUGGCUGCGUUGGCGGCCUGCCUAUGGGGCAACCCAUAGGACGCU | |
| | CUAAUACAGACAUGGUGCGAAGAGCCUAUUGAGCUAAUUGGUAGUCCUCCGGCCCCUGAAUGCGGCU | |
| | AAUCCUAACUGCGGAGCACAUGCCCUCAAACCAGGGGGUGGUGUGUCGUAACGGGUAACUCUGCAGC | |
| | GGAACCGACUACUUUGGGUGUCCGUGUUUCUUUUUAUUCUUAUAAUGGCUGCUUAUGGUGACAAUUA | |
| | AAGAAUUGUUACCAUAUAGCUAUUGGAUUGGCCAUCCGGUGACUAACAAAUCGCUCAUAUACCAGUU | |
| | UGUUGGUUUUGUUCCCUUAUCACAUACAGCUCAUAACACCCUCUUAUAUUUACUACAAUUGAAUAGC | |
| | AAGAA | |
| 126 | UAUGGCAGGCGGGCUUGUGGACGGCUUCGGCCCACCCACAGCAAGAAUGCCAUCAUCUGUCCUCACC | SalivirusNG- |
| | CCCAAUUUUCCCUUUUCUUCCCCUGCAACCAUUACGCUUACUCGCAUGUGCAUUGAGUGGUGCAUGU | J1 |
| | GUUGAACAAACAGCUACACUCACAUGGGGGCGGGUUUUCCCGCCCUACGGCCUCUCGCGAGGCCCAC | |
| | CCCUUCCCUCCCCUUAUAACUACAGUGCUUUGGUAGGUAAGCAUCCUGAUCCCCCGCGGAAGCUGCU | |
| | CACGUGGCAACUGUGGGGACCCAGACAGGUUAUCAAAGGCACCCGGUCUUUCCGCCUUCAGGAGUAU | |
| | CCCUACUAGUGAAUUCUAGCGGGGCUCUGCUUGGUGCCAACCUCCCCCAAAUGCGCGCUGCGGGAGU | |
| | GCUCUUCCCCAACUCACCCUAGUAUCCUCUCAUGUGUGUGCUUGGUCAGCAUAUCUGAGACGAUGUU | |
| | CCGCUGUCCCAGACCAGUCCAGUAAUGGACGGGCCAGUGCGUGUAGUCGUCUUCCGGCUUGUCCGGG | |
| | GCAUGUUUGGUGAACCGGUGGGGUAAGGUUGGUGUGCCCAACGCCCGUACUUUGGUGACACCUCAAG | |
| | ACCACCCAGGAAUGCCAGGGAGGUACCCCACCUCACGGUGGGAUCUGACCCUGGGCUAAUUGUCUAC | |
| | GGUGGUUCUUCUUGCUUCCACUUCUUUCUUCUGUUCACG | |
| 127 | CUUCCUUUUAAUUCGUAACUGAUAAGUGAUAGUCCUUGGAAGCUAGGUAUUUGUUACGCUAGUUUUG | PTVB |
| | GAUUAUCUUGUGCCCAACAUUUGUUUUCGAACAUAUGUUGUGUUUAAACACAGAAAUCUAGUUUCUU | |
| | UGGUUAUGAGUUUAAUGGAAUAUCCUUUUGAAAGACUUGCCUUGGCGCGGGCUAGAGCGCAAUUGUC | |
| | ACCAGGUAUUGCACCAAUGGUGGCGACAGGGUACAGAAGAGCAAGUACUCCUGACUGGGUAAUGGGA | |
| | CUGCAUUGCAUAUCCCUAGGCAUCUAUUGAGAUUUCUCUGGAGCCCACCAGCAUGGAGUUCCUGUAU | |
| | GGGAAUGCAGGACUGGACUUGUGCUGCCUGACAGGGUCGCGGCUGGCCGUCUGUACUUUGUAUAGUC | |
| | AGUUGAAACUCAUU | |
| 128 | UUUUGCGGCUCUGCCGCCGUUCGGGUUUUACCUGUUUUCACAGAGCAAAACAGGACCUCUAGUUUCG | BRAV-1 |
| | UGCUUAAACGAGAUCAUGCUCGAACUAGAACUAUAACGCUGGUCACUGGACCCGUGCCGCGCCUUGC | |
| | GGAUCUUUGCGGGAAUGGUGGCUAGUGGGCUGUGGAAGUGACUCUAACCACACGCCCCUCAAGUGUG | |
| | GGAAAACACGAACUGGUGUAGCGACGACGAUAGGCCUUGGGACACCCUCUCCAGUGAUGGAGACCCA | |
| | AGGGGCCAAAAGCCACGCCUUGUGCCCUGUCGUUCACAACCCCAGUGCAGUUCGUGCCAGUACCUGC | |
| | UUUUGGGAAGUGUGCUUUGGACAGCUGAAAACAGUCCUAGUGGGAGACUAAGGAUGCCCAGGAGGUA | |
| | CCCGGAGGUAACAAGUGACACUCUGGAUCUGACUUGGGGAGAGCGGGUCUGCUUUACAGACGCCACU | |
| | CUUUAAAAAACUUCUAUGUCUCGUCAGGCACCGGAGGCCGGGCCUUUUCCUUUAAAACAAUACACUU | |
| | U | |
| 129 | UUAAAACAGCUCUGGGGUUGUACCCACCCCAGAGGCCCACGUGGCGGCUAGUACUCCGGUAUUGCGG | PV1 |
| | UACCCUUGUACGCCUGUUUUAUACUCCCUUCCCGUAACUUAGACGCACAAAACCAAGUUCAAUAGAA | |
| | GGGGGUACAAACCAGUACCACCACGAACAAGCACUUCUGUUUCCCCGGUGAUGUCGUAUAGACUGCU | |
| | UGCGUGGUUGAAAGCGACGGAUCCGUUAUCCGCUUAUGUACUUCGAGAAGCCCAGUACCACCUCGGA | |
| | AUCUUCGAUGCGUUGCGCUCAGCACUCAACCCCAGAGUGUAGCUUAGGCUGAUGAGUCUGGACAUCC | |
| | CUCACCGGUGACGGUGGUCCAGGCUGCGUUGGCGGCCUACCUAUGGCUAACGCCAUGGGACGCUAGU | |
| | UGUGAACAAGGUGUGAAGAGCCUAUUGAGCUACAUAAGAAUCCUCCGGCCCCUGAAUGCGGCUAAUC | |
| | CCAACCUCGGAGCAGGUGGUCACAAACCAGUGAUUGGCCUGUCGUAACGCGCAAGUCCGUGGCGGAA | |
| | CCGACUACUUUGGGUGUCCGUGUUUCCUUUUAUUUUAUUGUGGCUGCUUAUGGUGACAAUCACAGAU | |
| | UGUUAUCAUAAAGCGAAUUGGAUUGGCCAUCCGGUGAAAGUGAGACUCAUUAUCUAUCUGUUUGCUG | |
| | GAUCCGCUCCAUUGAGUGUGUUUACUCUAAGUACAAUUUCAACAGUUAUUUCAAUCAGACAAUUGUA | |
| | UCAUA | |
| 130 | UUAAAACAGCGGAUGGGUAUCCCACCAUCCGACCCACAGGGUGUAGUGCUCUGGUAUUUUGUACCUU | HRVB27 |
| | UGCACGCCUGUUUCCCCAUUGUACCCCUCCUUAAAUUUCCUCCCCAAGUAACGUUAGAAGUUUAAGG | |
| | AAACAAAUGUACAAUAGGAAGCAUCACAUCCAGUGGUGUUAUGUACAAGCACUUCUGUUUCCCCGGA | |
| | GCGAGGUAUAAGUGGUACCCACCGCCGAAAGCCUUUAACCGUUAUCCGCCAAUCAACUACGUAAUGG | |
| | CUAGUAUUACCAUGUUUGUGACUUGGUGUUCGAUCAGGUGGUUCCCCCCACUAGUUUGGUCGAUGAG | |
| | GCUAGGAACUCCCCACGGGUGACCGUGUCCUAGCCUGCGUGGCGGCCAACCCAGCUUUUGCUGGGAC | |
| | GCCUUUUUACAGACAUGGUGUGAAGACCUGCAUGUGCUUGAUUGUGAGUCCUCCGGCCCCUGAAUGC | |
| | GGCUAACCUUAACCCCGGAGCCUUGCAACAUAAUCCAAUGUUGUUGAGGUCGUAAUGAGUAAUUCUG | |
| | GGAUGGGACCGACUACUUUGGGUGUCCGUGUUUCCUUUUAUUCUUUAUAUUGUCUUAUGGUCACAGC | |
| | AUAUAUAGCAUAUAUACUGUGAUC | |
| 131 | UUAAAACAGCGGAUGGGUAUCCCACCAUCCGGCCCACUGGGUGUAGUACUCUGGUACAUUGUACCUU | HRVB92 |
| | UGUACGCCUGUUUUCCCCCUCUUGUACCCGCCCUUCAAGCUCCUUGCCCAAGUAACGUUAGAAGUUU | |
| | GAACAUUGGUACAAUAGGAAGCAUCACAUCCAGUGGUGUACUGUACAAACACUUCUGUUGCCCCGGA | |
| | GCGAGGUAUAGAUGGUCCCCACCGUCAAAAGCCUUUAACCGUUAUCCGCCAAUCAACUACGUAAUGG | |
| | CUAGUAGCACCUUGGAUUUAAGUUGGCGUUCGAUCAGGUGGUAACCCCCACUAGUUUGGUCGAUGAG | |
| | GCUAGGAAUUCCCCACGGGUGACCGUGUCCUAGCCUGCGUGGCGGCCAACCCAGCAUCCGCUGGGAC | |
| | GCCAAUUUAAUGACAUGGUGUGAAGACCUGCAUGUGCUUGAUUGUGAGUCCUCCGGCCCCUGAAUGC | |
| | GGCUAACCCUAACCCCGGAGCCUUGCAGCACAAUCCAGUGUUGUUAAGGUCGUAAUGAGCAAUUCUG | |
| | GGAUGGGACCGACUACUUUGGGUGUCCGUGUUUCUUAUUUUUCUUGAAUUUUUCUUAUGGUCACAGC | |
| | AUAUAUACAUUAUAUACUGUGAUC | |
| 132 | UUAAAACAGCCUGUGGGUUGUUCCCACCCGCAGGGCCCACUGGGCGCUAGCACACUGGUAUCCCGGU | EVB107 |
| | ACCCUUGUGCGCCUGUUUUAUAUACCCUCCCCCUUAUGUAACUUAGAAGUAUGAUUCAAACGGUCGA | |
| | CAGGCGGCUCAGUGCACCAACUGAGUCAUGACCAAGCACUUCUGUUACCCCGGACUGAGUAUCAAUA | |
| | AGCUGUUCACACGGCUGAAGGAGAAAACGUUCGUUACCCGGCCAAUUACUUCGAGAAACCUAGUACC | |
| | ACCAUGAAGGUUGCGCAGUGUUUCGCUCCACACAACCCCAGUGUAGAUCAGGUCGAUGAGUCACCGC | |
| | AUUCCCCACGGGCGACCGUGGCGGUGGCUGCGUUGGCGGCCUGCCCAUGGGGCAACCCAUGGGACGC | |
| | UUCAAUACUGACAUGGUGUGAAGAGUCUAUUGAGCUAAUUGGUAGUCCUCCGGCCCCUGAAUGCGGC | |
| | UAAUCCCAACUGUGGAGCAGAUACUCACAAACCAGUGAGCGGUCUGUCGUAACGGGCAACUCCGCAG | |
| | CGGAACCGACUACUUUGGGUGUCCGUGUUUCUUUUUAUUCUUACAUUGGCUGCUUAUGGUGACAAUU | |
| | GACAAAUUGUUACCAUAUAGCUAUUGGAUUGGCCAUCCGGUGACAAACAGAGCUAUUGUUUACUUGU | |
| | UUGUUGGUUUCAUACCAUUAAAUUACAAGGUCUUAGAAACUCUCAACUUUAUUUUGACACUCAAUAC | |
| | AGCAAA | |
| 133 | UUGAAAGCCGGGGGUGGGAGAUCCGGAUUGCCAGUCUGCUCGAUAUCGCAGGCUGGGUCCGUGACUA | EMCV-A |
| | CCCACUCCCCCUUUCAACGUGAAGGCUACGAUAGUGCCAGGGCGGGUACUGCCGUAAGUGCCACCCC | |
| | AAAACAACAACAACCCCCCCUCUCCCUCCCCCCCCCCUAACGUUACUGGCCGAAGCCGCUUGGAAUA | |
| | AGGCCGGUGUGCGUUUGUCUAUAUGUUAUUUUCCACCAUAUUGCCGUCUUUUGGCAAUGUGAGGGCC | |
| | CGGAAACCUGGCCCUGUCUUCUUGACGAGCAUUCCUAGGGGUCUUUCCCCUCUCGCCAAAGGAAUGC | |
| | AAGGUCUGUUGAAUGUCGUGAAGGAAGCAGUUCCUCUGGAAGCUUCUUGAAGACAAACAACGUCUGU | |
| | AGCGACCCUUUGCAGGCAGCGGAACCCCCCACCUGGCGACAGGUGCCUCUGCGGCCAAAAGCCACGU | |
| | GUAUAAGAUACACCUGCAAAGGCGGCACAACCCCAGUGCCACGUUGUGAGUUGGAUAGUUGUGGAAA | |
| | GAGUCAAAUGGCUCUCCUCAAGCGUAUUCAACAAGGGGCUGAAGGAUGCCCAGAAGGUACCCCAUUG | |
| | UAUGGGAUCUGAUCUGGGGCCUCGGUGCACAUGCUUUACAUGUGUUUAGUCGAGGUUAAAAAACGUC | |
| | UAGGCCCCCCGAACCACGGGGACGUGGUUUUCCUUUGAAAAACACGAUGAUAAU | |
| 134 | UUGCCAGUCUGCUCGAUAUCGCAGGCUGGGUCCGUGACUACCCACUCCCCCUUUCAACGUGAAGGCU | EMCV-B |
| | ACGAUAGUGCCAGGGCGGGUACUGCCGUAAGUGCCACCCCAAACAACAACAACAAAACAAACUCCCC | |
| | CUCCCCCCCCUUACUAUACUGGCCGAAGCCACUUGGAAUAAGGCCGGUGUGCGUUUGUCUACAUGCU | |
| | AUUUUCUACCGCAUUACCGUCUUAUGGUAAUGUGAGGGUCCAGAACCUGACCCUGUCUUCUUGACGA | |
| | ACACUCCUAGGGGUCUUUCCCCUCUCGACAAAGGAGUGUAAGGUCUGUUGAAUGUCGUGAAGGAAGC | |
| | AGUUCCUCUGGAAGCUUCUUAAAGACAAACAACGUCUGUAGCGACCCUUUGCAGGCAGCGGAACCCC | |
| | CCACCUGGUGACAGGUGCCUCUGCGGCCAAAAGCCACGUGUAUAAGAUACACCUGCAAAGGCGGCAC | |
| | AACCCCAGUGCCACGUUGUGAGUUGGAUAGUUGUGGAAAGAGUCAAAUGGCUCUCCUCAAGCGUAUU | |
| | CAACAAGGGGCUGAAGGAUGCCCAGAAGGUACCCCAUUGUAUGGGAUCUGAUCUGGGGCCUCGGUGC | |
| | ACGUGCUUUACACGUGUUGAGUCGAGGUGAAAAAACGUCUAGGCCCCCCGAACCACGGGGACGUGGU | |
| | UUUCCUUUGAAAACCACGAUUACAAU | |
| 135 | UUGAAAGCCGGGGGUGGGAGAUCCGGAUUGCCAGUCUGCUCGAUAUCGCAGGCUGGGUCCGUGACUA | EMCV-PEC9 |
| | CCCACUCCCCCUUUCAACGUGAAGGCUACGAUAGUGCCAGGGCGGGUACUGCCGUAAGUGCCACCCC | |
| | AAAAUAACAACAGACCCCCCCCCUAACGUUACUGGCCGAAGCCGCUUGGAAUAAGGCCGGUGUGCGU | |
| | UUGUCUAUAUGUUAUUUUCCACCAUAUUGCCGUCUUUUGGCAAUGUGAGGGCCCGGAAACCUGGCCC | |
| | UGUCUUCUUGACGAGCAUUCCUAGGGGUCUUUCCCCUCUCGCCAAAGGAAUGCAAGGUCUGUUGAAU | |
| | GUCGUGAAGGAAGCAGUUCCUCUGGAAGCUUCUUGAAGACAAACAACGUCUGUAGCGACCCUUUGCA | |
| | GGCAGCGGAACCCCCCACCUGGCGACAGGUGCCUCUGCGGCCAAAAGCCACGUGUAUAAGAUACACC | |
| | UGCAAAGGCGGCACAACCCCAGUGCCACGUUGUGAGUUGGAUAGUUGUGGAAAGAGUCAAAUGGCUC | |
| | UCCUCAAGCGUAUUCAACAAGGGGCUGAAGGAUGCCCAGAAGGUACCCCAUUGUAUGGGAUCUGAUC | |
| | UGGGGCCUCGGUGCACAUGCUUUACAUGUGUUUAGUCGAGGUUAAAAAACGUCUAGGCCCCCCGAAC | |
| | CACGGGGACGUGGUUUUCCUUUGAAAAACACGAUGAUAAU | |
| 136 | UUAAAACUGGGUACAGGUUGUUCCCACCUGUAUCACCCACGUGGUGUGGUGCUCUUGUAUUCCGGUA | HRVC-02 |
| | CACUUGCACGCCAGUUUGCCACCCCUCACCCGUCGUAACUUAGAAGCUAACAACUCGACCAACAGGC | |
| | GGUGGUAAACCAUACCACUUACGGUCAAGCACUCCUGUUUCCCCGGUAUGCGAGGAAUAGACUCCUA | |
| | CAGGGUUGAAGCCUCAAGUAUCGUUAUCCGCAUUGGUACUACGCAAAGCUUAGUAGUGCCUUGAAAG | |
| | UCCCUUGGUUGGUCGCUCCGCUAGUUUCCCCUAGUAGACCUGGCAGAUGAGGCAGGACACUCCCCAC | |
| | UGGCGACAGUGGUCCUGCCUGCGUGGCUGCCUGCGCACCCUUAGGGGUGCGAAGCCAAGUGACAGAC | |
| | AAGGUGUGAAGAGCCCCGUGUGCUACCAAUGAGUCCUCCGGCCCCUGAAUGCGGCUAAUCCAACCCC | |
| | ACAGCUAUUGCACACAAGCCAGUGUGUAUGUAGUCGUAAUGAGCAAUUGUGGGACGGAACCGACUAC | |
| | UUUGGGUGUCCGUGUUUCCUUUUAUUCUUAUCAUUCUGCUUAUGGUGACAAUACUGUGAAAUAGUGU | |
| | UGUUACC | |
| 137 | UAAAACUGGAUCCAGGUUGUUCCCACCUGGAUCUCCUAUUGGGAGUUGUACUCUAUUAUUCCGGUAA | HRV-A21 |
| | UUUUGUACGCCAGUUUUAUCUUCCCCCUCCCCAAUUGUAACUUAGAAGGUUAUCAAUACGACCAAUA | |
| | GGUGGUAGUUAGCCAAACUACCAAAGGUCAAGCACUUCUGUUUCCCCGGUCAAAGUUGAUAUGCUCC | |
| | AACAGGGCAAAAACAACUGAGAUCGUUAUCCGCAAAGUGCCUACGCAAAGCCUAGUAACACCUUUGA | |
| | AGAUUUAUGGUUGGUCGUUCCGCUAUUUCCCAUAGUAGACCUGGCAGAUGAGGCUAGAAAUCCCCCA | |
| | CUGGCGACAGUGCUCUAGCCUGCGUGGCUGCCUGCGCACCCCUUGGGUGCGAAGCCAUACAUUGGAC | |
| | AAGGUGUGAAGAGCCCCGUGUGCUCACUUUGAGUCCUCCGGCCCCUGAAUGUGGCUAACCUUAACCC | |
| | UGCAGCUAGUGCAUGUAAUCCAACAUGUUGCUAGUCGUAAUGAGUAAUUGCGGGACGGGACCAACUA | |
| | CUUUGGGUGUCCGUGUUUCACUUUUUCCUUUUAAUAUUGCUUAUGGUGACAAUAUAUAUAGCUAUAU | |
| | AUAUUGACACC | |
| 138 | UUCCCCUGCAACCAUUACGCUUACUCGCAUGUGCAUUGAGUGGUGCAUGUGUUGAACAAACAGCUAC | Salivirus-A- |
| | ACUCACAUGGGGGCGGGUUUUCCCGCCCUACGGCUUCUCGCGAGGCCCACCCCUCCCCUUUCUCCCA | SH1 |
| | UAACUACAGUGCUUUGGUAGGUAAGCAUCCUGAUCCCCCGCGGAAGCUGCUCACGUGGCAACUGUGG | |
| | GGACCCAGACAGGUUAUCAAAGGCACCCGGUCUUUCCGCCUUCAGGAGUAUCCCUGCUAGCGAAUUC | |
| | UAGUAGGGCUCUGCUUGGUGCCAACCUCCCCCAAAUGCGCGCUGCGGGAGUGCUCUUCCCCAACUCA | |
| | CCCUAGUAUCCUCUCAUGUGUGUGCUUGGUCAGCAUAUCUGAGACGAUGUUCCGCUGUCCCAGACCA | |
| | GUCCAGUAAUGGACGGGCCAGUGUGCGUAGUCGUCUUCCGGCUUGUCCGGCGCAUGUUUGGUGAACC | |
| | GGUGGGGUAAGGUUGGUGUGCCCAACGCCCGUACUCAGGGGAUACCUCAAGGCACCCAGGAAUGCCA | |
| | GGGAGGUACCCCGCUUCACAGCGGGAUCUGACCCUGGGGUAAAUGUCUGCGGGGGGUCUUCUUGGCC | |
| | CACUUCUCAGUACUUUUCAGG | |
| 139 | UUAAAACAGCCUGUGGGUUGUUCCCAUCCACAGGGCCCACUGGGCGCCAGCACUCUGGUAUUGCGGU | Echovirus- |
| | ACCUUAGUGCGCCUGUUUUAUAUACCCGUCCCCCAAACGUAACUUAGACGCAUGUCAACGAAGACCA | E14 |
| | AUAGUAAGCGCAGCACACCAGCUGUGUUCCGGUCAAGCACUUCUGUUACCCCGGACCGAGUAUCAAU | |
| | AAGCUACUCACGUGGCUGAAGGAGAAAACGUUCGUUACCCGACCAAUUACUUCAAGAAACCUAGUAA | |
| | CACCAUGAAGGUUGCGCAGUGUUUCGCUCCGCACAACCCCAGUGUAGAUCAGGUCGAUGAGUCACCG | |
| | CAUUCCCCACGGGUGACCGUGGCGGUGGCUGCGCUGGCGGCCUGCCCAUGGGGAAACCCAUGGGACG | |
| | CUUCAAUACUGACAUGGUGCGAAGAGUCUAUUGAGCUAAUUGGUAGUCCUCCGGCCCCUGAAUGCGG | |
| | CUAAUCCUAACUGCGGAGCAGAUACCCACACACCAGUGGGCAGUCUGUCGUAACGGGCAACUCUGCA | |
| | GCGGAACCGACUACUUUGGGUGUCCGUGUUUCUCUUUAUCCUUAUACUGGCUGCUUAUGGUGACAAU | |
| | UGAGAGAUUGUUACCAUAUAGCUAUUGGAUUGGCCAUCCGGUGACAAAUAGAGCAAUUGUGUAUUUG | |
| | UUUGUUGGUUUCGUGCCAUUAAAUUACAAGGUUCUAAACACCCUUAAUCUUAUUAUAGCAUUCAACA | |
| | CAACAAA | |
| 140 | GCUCUUCCGAUCUGGGUUGUUCCCACCCACAGGGCCCACUGGGCGCCAGCACUCUGAUUCCACGGAA | CVA10 |
| | UCUUUGUGCGCCUGUUUUACAACCCUUCCCAAUUUGUAACGUAGAAGCAAUACACACUACUGAUCAA | |
| | UAGUAGGCAUGGCGCGCCAGUCAUGUCAUGAUCAAGCACUUCUGUUCCCCCGGACUGAGUAUCAAUA | |
| | GACUGCUCACGCGGUUGAAGGAGAAAACGUUCGUUACCCGGCUAACUACUUCGAGAAACCUAGUAGC | |
| | ACCAUGGAAGCUGCGGAGUGUUUCGCUCAGCACUUUCCCCGUGUAGAUCAGGUCGAUGAGUCACUGC | |
| | AAUCCCCACGGGCGACCGUGGCAGUGGCUGCGUUGGCGGCCUGCCUAUGGGGCAACCCAUAGGACGC | |
| | UCUAAUGUGGACAUGGUGCGAAGAGUCUAUUGAGCUAGUUAGUAGUCCUCCGGCCCCUGAAUGCGGC | |
| | UAAUCCUAACUGCGGAGCACAUGCCUUCAACCCAGGAGGUGGUGUGUCGUAACGGGUAACUCUGCAG | |
| | CGGAACCGACUACUUUGGGUGUCCGUGUUUCCUUUUAUCCUUAUAUUGGCUGCUUAUGGUGACAAUC | |
| | ACGGAAUUGUUGCCAUAUAGCUAUUGGAUUGGCCAUCCGGUGUCUAACAGAGCUAUUGUAUACCUAU | |
| | UUGUUGGAUUUACUCCCCUAUCAUACAAAUCUCUGAACACUUUGUGCUUUAUACUGAACUUAAACAC | |
| | ACGAAA | |
| 141 | UUAAAACAGCUCUGGGGUUGUUCCCACCCCAGAGGCCCACGUGGCGGCCAGUACACCGGUACCACGG | Enterovirus- |
| | UACCCUUGUACGCCUGUUUUAUACUCCCCUCCCCGUAAACUAGAAGCACGAAACACAAGUUCAAUAG | C |
| | AAGGGGGUACAGACCAGUACCACCACGAACAAGCACUUCUGUUCCCCCGGUGAGGUCACAUAGACUG | |
| | UCCCCACGGUCAAAAGUGACUGAUCCGUUAUCCGCUCACGUACUUCGGAAAGCCUAGUACCACCUUG | |
| | GAAUCUACGAUGCGUUGCGCUCAGCACUCGACCCCGGAGUGUAGCUUAGGCUGAUGAGUCUGGACGU | |
| | UCCCCACUGGUGACAGUGGUCCAGGCUGCGUUGGCGGCCUACCUGUGGUCCAAAACCACAGGACGCU | |
| | AGUAGUGAACAAGGUGUGAAGAGCCCACUGAGCUACCUGAGAAUCCUCCGGCCCCUGAAUGCGGCUA | |
| | AUCCCAACCACGGAGCAGGUAAUCGCAAACCAGCGGUCAGCCUGUCGUAACGCGUAAGUCUGUGGCG | |
| | GAACCGACUACUUUGGGUGUCCGUGUUUCCUUUUAUUUUUAUGGUGGCUGCUUAUGGUGACAAUCAU | |
| | AGAUUGUUAUCAUAAAGCAAAUUGGAUUGGCCAUCCGGAGUGAGCUAAACUAUCUAUUUCUCUGAGU | |
| | GUUGGAUUCGUUUCACCCACAUUCUGAACAAUCAGCCUCAUUAGUGUUACCCUGUUAAUAAGACGAU | |
| | AUCAUCACG | |
| 142 | UUAAAAUAGCCUCAGGGUUGUUCCCACCCUGAGGGCCCACGUGGUGUAGUACUCUGGUAUUACGGUA | Enterovirus- |
| | CCUUUGUACGCCUAUUUUAUACCCCCUUCCCCAAGUAAUUUAGAAGCAAGCACAAACCAGUUCAGUA | J |
| | GUAAGCAGUACAAUCCAGUACUGUAAUGAACAAGUACUUCUGUUACCCCGGAAGGGUCUAUCGGUAA | |
| | GCUGUACCCACGGCUGAAGAAUGACCUACCGUUAACCGGCUACCUACUUCGAGAAGCCUAGUAAUGC | |
| | CGUUGAAGUUUUAUUGACGUUACGCUCAGCACACUACCCCGUGUGUAGUUUUGGCUGAUGAGUCACG | |
| | GCACUCCCCACGGGCGACCGUGGCCGUGGCUGCGUUGGCGGCCAACCAAGGAGUGCAAGCUCCUUGG | |
| | ACGUCAUAUUACAGACAUGGUGUGAAGAGCCUAUUGAGCUAGGUGGUAGUCCUCCGGCCCCUGAAUG | |
| | CGGCUAAUCCUAACUCCGGAGCAUAUCGGUGCGAACCAGCACUUGGUGUGUUGUAAUACGUAAGUCU | |
| | GGAGCGGAACCGACUACUUUGGGUGUCCGUGUUUCCUGUUUUAACUUUUAUGGCUGCUUAUGGUGAC | |
| | AAUUUAACAUUGUUACCAUAUAGCUGUUGGGUUGGCCAUCCGGAUUUUGUUAUAAAACCAUUUCCUC | |
| | GUGCCUUGACCUUUAACACAUUUGUGAACUUCUUUAAAUCCCUUUUAUUAGUCCUUAAAUACUAAGA | |
| 143 | CCCCUCCACCCUUAAGGUGGUUGUAUCCCACAUACCCCACCCUCCCUUCCAAAGUGGACGGACAACU | Sapelovirus |
| | GGAUUUUGACUAACGGCAAGUCUGAAUGGUAUGAUUUGGAUACGUUUAAACGGCAGUAGCGUGGCGA | |
| | GCUAUGGAAAAAUCGCAAUUGUCGAUAGCCAUGUUAGUGACGCGCUUCGGCGUGCUCCUUUGGUGAU | |
| | UCGGCGACUGGUUACAGGAGAGUAGGCAGUGAGCUAUGGGCAAACCUCUACAGUAUUACUUAGAGGG | |
| | AAUGUGCAAUUGAGACUUGACGAGCGUCUCUUUGAGAUGUGGCGCAUGCUCUUGGCAUUACCAUAGU | |
| | GAGCUUCCAGGUUGGGAAACCUGGACUGGGCCUAUACUACCUGAUAGGGUCGCGGCUGGCCGCCUGU | |
| | AACUAGUAUAGUCAGUUGAAACCCCCCC | |
| 144 | UUUCCUCCUUUCGACCGCCUUACGGCAGGCGGGUCCGCGGACGGCUUCGGCCUACCCGCGACAAGAA | Salivirus-A- |
| | UGCCGUCAUCUGUCCUUAUCACCCAUAUUCUUUCCCUUCCCCCGCAACCAUCACGCUUACUCGCGCA | BN5 |
| | CGUGUUGAGUGGCACGUGCGUUGUCCAAACAGUUACACUCACACCCUUGGGGGGGGUUUGUCCCGCC | |
| | CUCGGGUUCCUCGCGGAACCCUCCCUCUUCUCUCUCCCUUUCUAUCCGCCUUCACUUUCCAUAACUA | |
| | CAGUGCUUUGGUAGGUAAGCAUCCUGACCCCCCGCGGAAGCUGCCAACGUGGCAACUGUGGGGAUCC | |
| | AGGCAGGUUAUCAAAGGCACCCGGUCUUUCCGCCUUCAGGAGUAUCCCUGCCGGUGAAUUCCGACAG | |
| | GGCUCUGCUUGGUGCCAACCUCCCCCAAAUGCGCGCUGCGGGAGUGCUCUUCCCCAACUCAUCUUAG | |
| | UAACCUCUCAUGUGUGUGCUUGGUCAGCAUAUCUGAGGCGACGUUCCGCUGUCCCAGACCAGUCCAG | |
| | CAAUGGACGGGCCAGUGUGCGUAGUCGCUUUCCGGUUUCCCGGCGCAUGUUUGGCGAAACGCUGAGG | |
| | UAAGGUUGGUGUGCCCAAUGCCCGUAAUUUGGUGACACCUCAAGACCACCCAGGAAUGCCAGGGAGG | |
| | UACCCCACUUCGGUGGGAUCUGACCCUGGGCUAAUUGUCUACGGUGGUUCUUCUUGCUUCCACUUCU | |
| | CUUUUUUCUGGCAUG | |
| 145 | UAUGGCAGGCGGGCUUGUGGACGGCUUCGGCCCACCCACAGCAAGAAUGCCAUCAUCUGUCCUCACC | Salivirus-A- |
| | CCCAUGUUUCCCCUUUCUUUCCCUGCAACCGUUACGCUUACUCGCAGGUGCAUUUGAGUGGUGCACG | BN2 |
| | UGUUGAAUAAACAGCUACACUCACAUGGGGGCGGGUUUUCCCGCCCUGCGGCCUCUCGCGAGGCCCA | |
| | CCCCUCCCCUUCCUCCCAUAACUACAGUGCUUUGGUAGGUAAGCAUCCUGAUCCCCCGCGGAAGCUG | |
| | CUCACGUGGCAACUGUGGGGACCCAGACAGGUUAUCAAAGGCACCCGGUCUUUCCGCCUUCAGGAGU | |
| | AUCCCUGCUAGUGAAUUCUAGUAGGGCUCUGCUUGGUGCCAACCUCCCCCAAAUGCGCGCUGCGGGA | |
| | GUGCUCUUCCCCAACUCACCCUAGUAUCCUCUCAUGUGUGUGCUUGGUCAGCAUAUCUGAGACGAUG | |
| | UUCCGCUGUCCCAGACCAGUCCAGUAAUGGACGGGCCAGUGUGCGUAGUCGUCUUCCGGCUUUUCCG | |
| | GCGCAUGUUUGGUGAACCGGUGGGGUAAGGUUGGUGUGCCCAACGCCCGUACUUUGGUGAUACCUCA | |
| | AGACCACCCAGGAAUGCCAGGGAGGUACCCCGCUUCACAGCGGGAUCUGACCCUGGGCUAAUUGUCU | |
| | ACGGUGGUUCUUCUUGCUUCCACUUCUUUCUACUGUUC | |
| 146 | UAUGGCAGGCGGGCUUGUGGACGGUUUCGGCCCACCCACAGCAAGAAUGCCAUCAUCUGUCCUCACC | Salivirus-A- |
| | CCCAAUUUUCCCUUUCUUCCCCUGCAAUCAUCACGCUUACUCGCAUGUGCAUUGAGUGGUGCAUGUG | GUT |
| | UUGAACAAACAGCUACACUCACAUGGGGGCGGGUUUUCCCGCCCUACGGCCUCUCGCGAGGCCCACC | |
| | CUUCCCCUCCCCUUAUAACUACAGUGCUUUGGCAGGUAAGCAUCCUGAUCCCCCGCGGAAGCUGCUC | |
| | ACGUGGCAACUGUGGGGACCCAGACAGGUUAUCAAAGGCACCCGGUCUUUCCGCCUUCAGGAGCAUC | |
| | CCCACUAGUGAAUUCUAGUGGGGCUCUGCUUGGUGCCAACCUCCCCCAAAUGCGCGCUGCGGGAGUG | |
| | CUCUUCCCCAACCCAUCCUAGUAUCCUCUCAUGUGUGUGCUUGGUCAGCAUAUCUGAGACGACGUUC | |
| | CGCUGUCCCAGACCAGUCCAGUAAUGGACGGGCCAGUGUGCGUAGUCGUCUUCCGGCUUGUCCGGCG | |
| | CAUGUUUGGUGAACCGGUGGGGUAAGGUUGGUGUGCCCAACGCCCGUACUUUGGUGACACCUCAAGA | |
| | CCACCCAGGAAUGCCAGGGAGGUACCCCGCCUCACGGCGGGAUCUGACCCUGGGCUAAUUGUCUACG | |
| | GUGGUUCUUCUUGCUUCCACUUCUUUCUU | |
| 147 | UUCUCCUGCAACCAUUACGCUUAAUCGCAUGUGCAUUGAGUGGUGCAUGUGUUGAACAAACAGCUAC | Salivirus-A- |
| | AAUCACAUGGGGGGGGGUUUUCCCGCCCCACGGCUUCUCGCGAGGCCCAUCCCUCCCUUUUCUCCCA | CH |
| | UAACUACAGUGCUUUGGUAGGUAAGCAUCCCGAUCUCCCGCGGAAGCUGCUCACGUGGCAACUGUGG | |
| | GGACCCAGACAGGUUAUCAAAGGCACCCGGUCUUUCCGCCUUCAGGAGUAUCCCUGCUAGCGAAUUC | |
| | UAGUAGGGCUCUGCUUGGUGCCAACCUCUCCCAAAUGCGCGCUGCGGGAGUGCUCUUCCCCAAAUCA | |
| | CCCCAGUAUCCUCUCAUGUGUGUGCCUGGUCAGCAUAUCUGAGACGAUGUUCCGCUGUCCCAGACCA | |
| | GUCCAGUAAUGGACGGGCCAGUGUGCGUAGUCGUCCUCCGGCUUGUCCGGCGCAUGUUUGGUGAACC | |
| | GGUGGGGUAAGGUUGGUGUGCCCAACGCCCGUAAUCAGGGGAUACCUCAAGGCACCCAGGAAUGCCA | |
| | GGGAGGUAUCCCGCCUCACAGCGGGAUCUGACCCUGGGGUAAAUGUCUGCGGGGGGUCCUCUUGGCC | |
| | CAAUUCUCAGUAAUUUUCAGG | |
| 148 | UCUGUCCUCACCCCAUCUUCCCUUCUUUCCUGCACCGUUACGCUUACUCGCAUGUGCAUUGAGUGGU | Salivirus-A- |
| | GCACGUGCUUGAACAAACAGCUACACUCACAUGGGGGCGGGUUUUCCCGCCCUGCGGCCUCUCGCGA | SZ1 |
| | GGCCCACCCCUCCCCUUCCUCCCAUAACUACAGUGCUUUGGUAGGUAAGCAUCCUGAUCCCCCGCGG | |
| | AAGCUGCUCACGUGGCAACUGUGGGGACCCAGACAGGUUAUCAAAGGCACCCGGUCUUUCCGCCUUC | |
| | AGGAGUAUCCCUGCUAGUGAAUUCUAGUAGGGCUCUGCUUGGUGCCAACCUCCCCCAAAUGCGCGCU | |
| | GCGGGAGUGCUCUUCCCCAACUCACCCUAGUAUCCUCUCAUGUGUGUGCUUGGUCAGCAUAUCUGAG | |
| | ACGAUGUUCCGCUGUCCCAGACCAGUCCAGUAAUGGACGGGCCAGUGUGCGUAGUCGUCUUCCGGCU | |
| | UGUCCGGCGCAUGUUUGGUGAACCGGUGGGGUAAGGUUGGUGUGCCCAACGCCCGUACUUUGGUGAU | |
| | ACCUCAAGACCACCCAGGAAUGCCAGGGAGGUACCCCGCUUCACAGCGGGAUCUGACCCUGGGCUAA | |
| | UUGUCUACGGUGGUUCUUCUUGCUUCCACUUCUUUCUACUGUUCAUG | |
| 149 | UUAAAACAGCCUGUGGGUUGUUCCCACCCACAGGGCCCAUUGGGCGUCAGCACCCUGGUAUCACGGU | Echovirus-7 |
| | ACCUUUGUGCGCCUGUUUUAUAUCCCUUCCCCCAAUUGUAACUUAGAAGAAACACACACCGAUCAAC | |
| | AGCAAGCGUGGCACACCAGCCAUGUUUUGGUCAAGCACUUCUGUUACCCCGGACUGAGUAUCAAUAG | |
| | ACUGCUCACGCGGUUGAAGGAGAAAGCGUCCGUUAUCCGGCCAGCUACUUCGAGAAACCUAGUAACA | |
| | CCAUGGAAGUUGCGGAGUGUUUCGCUCAGCACUACCCCAGUGUAGAUCAGGUCGAUGAGUCACCGCU | |
| | UUCCCCACGGGCGACCGUGGCGGUGGCUGCGUUGGCGGCCUGCCUAUGGGGGAACCCAUAGGACGCU | |
| | CUAAUACAGACAUGGUGCGAAGAGUCUAUUGAGCUAGCUGGUAUUCCUCCGGCCCCUGAAUGCGGCU | |
| | AAUCCUAACUGUGGAGCACAUGCCCCUAAUCCAAGGGGUAGUGUGUCGUAAUGAGCAAUUCCGCAGC | |
| | GGAACCGACUACUUUGGGUGUCCGUGUUUCCUCUUAUUCUUGUACUGGCUGCUUAUGGUGACAAUUG | |
| | AGAGAUUGUUACCAUAUAGCUAUUGGAUUGGCCAUCCGGUGACUAAUAGAGCUAUUGUGUAUCUCUU | |
| | UGUUGGAUUUGUACCACUUAAUUUGAAAGAAAUCAGGACACUACGCUACAUUUUACUAUUGAACACC | |
| | GCAAA | |
| 150 | UUAAAACAGCCUGUGGGUUGCACCCACUCACAGGGCCCACUGGGCGCAAGCACUCUGGCACUUCGGU | EVA71 |
| | ACCUUUGUGCGCCUGUUUUAUAUCCCCUCCCCCAAUGAAAUUUAGAAGCAGCAAACCCCGAUCAAUA | |
| | GCAGGCAUAACGCUCCAGUUAUGUCUUGAUCAAGCACUUCUGUUUCCCCGGACUGAGUAUCAAUAGA | |
| | CUGCUCACGCGGUUGAAGGAGAAAACGUUCGUUAUCCGGCUAACUACUUCGGAAAGCCUAGUAACAC | |
| | CAUGGAAGUUGCGGAGAGUUUCGUUCAGCACUUCCCCAGUGUAGAUCAGGUCGAUGAGUCACCGCAU | |
| | UCCCCACGGGCGACCGUGGCGGUGGCUGCGUUGGCGGCCUGCCCAUGGGGUAACCCAUGGGACGCUC | |
| | UAAUACGGACAUGGUGUGAAGAGUCUACUGAGCUAGUUAGUAGUCCUCCGGCCCCUGAAUGCGGCUA | |
| | AUCCCAACUGCGGAGCACACGCCCACAAGCCAGUGGGUAGUGUGUCGUAACGGGCAACUCUGCAGCG | |
| | GAACCGACUACUUUGGGUGUCCGUGUUUCCUUUUAUUCUUAUGUUGGCUGCUUAUGGUGACAAUUAA | |
| | AGAGUUGUUACCAUAUAGCUAUUGGAUUGGCCAUCCGGUGUGCAACAGAGCGAUCGUUUACCUAUUU | |
| | AUUGGUUUUGUACCAUUGACACUGAAGUCUGUGAUCACCCUUAAUUUUAUCUUAACCCUCAACACAG | |
| | CCAAAC | |
| 151 | CCUCGGUCCCUCUUUCCGUCGCCGCCCACGACGUUAAAUGCGGUGUUGUGGUGCUUAGGUGCCACAC | Oscivirus |
| | CACUGCUAUUUGGGUCCCCCUUUCCCCUAUAUAUGUUUGUUUGUUUAUUUCAAUUUCUUGAGGAUUG | |
| | GCACCUCCUUAUGCCAAAUCUAAAUCGUGGAGGAUCCCAGGCUUUCUGGUCUUUAACAGAACUCCAC | |
| | GUCCAGGUCAUAGAAACUGGUUGGUAGGCUGCCUGAGUAGUCCAUUUGCUAGUAGUCCCUUGUGAAC | |
| | AGGGUGGCUCCCGUUUACUGCUGGUAUUCCCGGUGUAGGUCGCCAUGGUGGUAACACCAUCCUGCAU | |
| | UGUGUGUGAACCAGUACCGCAAGGAUAGCAAGGUAUGAACACUUGUGGACGAAAUGGUAAGUGAUCA | |
| | AUUCACUUUCAUGGCCGGAAGGUCACGUGGCAAUCAUGCCACCCAGGUACCCUCCUCUGGGAGGAUC | |
| | UGAGGGUGGGCUAAGCAGACCCUGCCAUGUGGCUGAACUUUUCCCUUAUUGUUUUACUUUGUAACAU | |
| | UUAUAGUUGUGUUAGUGAUUUGUGUGUUGUGCCCUUGUGAGCUAUAUCCAGUAUAAGUUCGCAGCUA | |
| | GAAGUUAAUCCUUCGACAUCGGCUGUAUUGGAA | |
| 152 | GUUAUCCGCUUAUGUACUUCGAGAAGCCCAGUACCACCUCGGAAUCUUCGAUGCGUUGCGCUCAGCA | PV-Mahoney |
| | CUCAACCCCAGAGUGUAGCUUAGGCUGAUGAGUCUGGACAUCCCUCACCGGUGACGGUGGUCCAGGC | |
| | UGCGUUGGCGGCCUACCUAUGGCUAACGCCAUGGGACGCUAGUUGUGAACAAGGUGUGAAGAGCCUA | |
| | UUGAGCUACAUAAGAAUCCUCCGGCCCCUGAAUGCGGCUAAUCCCAACCUCGGAGCAGGUGGUCACA | |
| | AACCAGUGAUUGGCCUGUCGUAACGCGCAAGUCCGUGGCGGAACCGACUACUUUGGGUGUCCGUGUU | |
| | UCCUUUUAUUUUAUUGUGGCUGCUUAUGGUGACAAUCACAGAUUGUUAUCAUAAAGCGAAUUGGAUU | |
| | GGCCAUCCGGUGAAAGUGAGAUUCAUUAUCUAUCUGUUUGCUGGAUUCGCUCCAUUGAGUGUGUUUA | |
| | CUCUAAGUACAAUUUCAACAGUUAUUUCAAUCAGACAAUU | |
| 153 | CACCCAUACACCCCCACCCCCUUUUCUGUAACUCAAGUAUGUGUGCUCGUAAUCUUGACUCCCACGG | Aichivirus- |
| | AAUGGAUCGAUCCGCUGGAGAACAAACUGCUAGAUCCACAUCCUCCCUCCCCUUGGGAGGACCUCGG | A-FSS693 |
| | UCCUCCCACAUCCUCCCUCCAGCCUGACGUAUCACAGGCUGUGUGAAGCCCCCGCGAAAGCUGCUCA | |
| | CGUGGCAAUUGUGGGUCCCCCCUUCAUCAAGACACCAGGUCUUUCCUCCUUAAGGCUAGCCCCGAUG | |
| | UGUGAAUUCACAUUGGGCAACUAGUGGUGUCACUGUGCGCUCCCAAUCUCGGCCGCGGAGUGCUGUU | |
| | CCCCAAGCCAAACCCCUGGCCCUUCACUAUGUGCCUGGCAAGCAUAUCUGAGAAGGUGUUCCGCUGU | |
| | GGCUGCCAGCCUGGUAACAGGUGCCCCAGUGUGCGUAACCUUCUUCCGUCUCCGGACGGUAGUGAUU | |
| | GGUUAAGAUUUGGUGUAAGGUUCAUGUGCCAACGCCCUGUGCGGGAUGAAACCUCUACUGCCCUAGG | |
| | AAUGCCAGGCAGGUACCCCACCUUCGGGUGGGAUCUGAGCCUGGGCUAAUUGUCUACGGGUAGUUUC | |
| | AUUUCCAAUUCUUUUAUGCUGGAGUC | |
| 154 | GUGUUACUCCAUUCAGCUUCUUCGGAACCUGUUCGGAGGAAUUAAACGGGCACCCAUACUCCCCCCC | Aichivirus- |
| | ACCCCCCUUUUGUAACUAAGUAUGUGUGCUCGUGACCUUGACUCCCACGGAACGGACCGAUCCGUUG | KVGH |
| | GUGAACAAACAGCUAGGUCCACAUCCUCCUUUCCCCUGGGAGGGUCCCCGCCCUCCCACAUCCCCCC | |
| | CCCAGCCUGACGUGUCACAGGCUGUGUGAAGCCCCCGCGAAAGCUGCUCACGUGGCAAUUGUGGGUC | |
| | CCCCCUUCAUCAAGACACCAGGUCUUUCCUCCUUAAGGCUAGCCCCGGCGUGUGAACUCACGUUGGG | |
| | CAACUAGUGGUGUCACUGUGCGCUCCCAAUCUCGGCCGCGGAGUGCUGUUCCCCAAGCCAAACCCCU | |
| | GGCCCUUCACUAUGUGCCUGGCAAGCACACCUGAGAAGGUGUUCCGCUGUGGCUGCCAGCCUGGUAA | |
| | CAGGUGCCCCAGUGUGCGUAACCUUCUUCCGUCUUCGGACGGUGGUGAUUGGUUAAGAUUUGGUGUA | |
| | AGGUUCAUGUGCCAACGCCCUGUGCGGGAUGAAACCUCUACUGCCCUAGGAAUGCCAGGCAGGUACC | |
| | CCACCUUCGGGUGGGAUCUGAGCCUGGGCUAAUUGUCUACGGGUGGUUUCAUUUCCAAUUCUUUCAU | |
| | GUCGGAGUC | |
| 155 | GUUACUCCAUUCAGCUUCUUCGGAACCUGUUCGGAGGAAUUAAACGGGCACCCAUACACCCCCACCC | Aichivirus- |
| | CCUUUUCUGCAACUUAAGUAUGUGUGCUCGUAAUCUUGACUCCCACGGAACGGAUCGAUCCGCUGGA | DV |
| | GAACAAACUGCUAGAUCCACAUCCUCCCUUCCCCUGGGAGGACCCCGGUCCUCCCACAUCCUCCCCC | |
| | CAGCCUGACGUAACACAGGCUGUGUGAAGUCCCCGCGAAAGCUGCUCACGUGGCAAUUGUGGGUCCC | |
| | CCCUUCACCAAGACACCAGGUCUUUCCUCCUUAAGGCUAGCCCCGAUGUGUGAAUUCACAUUGGGCA | |
| | ACUAGUGGUGUCACUGUGCGCUCCCAAUCUCGGCCGCGGAGUGCUGUUCCCCAAGCCAAACCCCUGG | |
| | CCCUUCACUAUGUGCCUGGCAAGCAUAUCUGAGAAGGUGUUCCGCUGUGGCUGCCAGCCUGGUAACA | |
| | GGUGCCCCAGUGUGCGUAACCUUCUUCCGUCUCCGGACGGUAGUGAUUGGUUAAGAUUUGGUGUAAG | |
| | GUUCAUGUGCCAACGCCCUGUGCGGGAUGAAACCUCUACUGCCCUAGGAAUGCCAGGCAGGUACCCC | |
| | ACCUUCGGGUGGGAUCUGAGCCUGGGCUAAUUGUCUACGGGUAGUUUCAUUUCCAAUUCUUUUAUGU | |
| | CGGAGUC | |
| 156 | GUAACUUCAAGUGUGUGUGCUCGUAAUCUUGACUCCUGCCGGAAUGCCGCCCGGUUCAGUGAACAAA | Murine- |
| | CAGCUAGGCAAGUCCCUCCCUUCCCCUGUGGUCGGUUCUCACCGGCCACCAUCCCUCCCCCAGCCUG | Kobuvirus-1- |
| | ACGUGUUACAGGCUGUGCAAAGCCCCCGCGAAAGCUGCUCACGUGGCAAUUGUGGGUCCCCCCUUUG | aichivirusA3 |
| | UCAAGACACCGAGUCUUUCUCCCUUAAGGCUAGCCCGGUCCCACGAACGUGGAACUGGCAACUAGUG | |
| | GUGUCACUACACGCCUCCGACCUCGGACGCGGAGUGCUGUUCCCCAAGCUGUAACCCUGACCCAAGA | |
| | CUGUGCUGCCUGGCAAGCACCGUCUGGGAAGAUGUUCCGCUGUGGCUGCCAAACCUGGUAACAGGUG | |
| | CCCCAGUGUGUGUAGUCUUCCUCCAGUCUCCGGACUGGCAGUCUUGUGUAAAGAUGCAGUGUAAGGU | |
| | UCAAGUGCCAAAUCCCUGGAAGGAGUGACCCUCUACUGCCCUAGGAAUGCUGUGCAGGUACCCCCAA | |
| | CUUCGGUUGGGGAUCUGAGCACAGGCUAAUUGUCUACGGGUAGUUUCAUUUCCCAUCCUCUCUUUUU | |
| | UGGCAUC | |
| 157 | UUUGAAAAGGGGGUGGGGGGGCCUCGGCCCCCUCACCCUCUUUUCCGGUGGCCACCCGCCCGGGCCA | Porcine- |
| | CCGUUACUCCACUCCACUCCUUCGGGACUGGUUUGGAGGAACAUAACAGGGCUUCCCAUCCCUGUUU | Kobuvirus-K- |
| | ACCCUUACUCCACUCACCCCUCCCCUUGACCAACCCUAUCCACACCCCACUGACUGACUCCUUUGGA | 30 |
| | UCUUGACCUCGGAAUGCCUACUUGACCUCCCACUUGCCUCUCCCUUUUCGGAUUGCCGGUGGUGCCU | |
| | GGCGGAAAAAGCACAAGUGUGUUGUUGGCUACCAAACUCCUACCCGACAAAGGUGCGUGUCCGCGUG | |
| | CUGAGUAAUGGGAUAGGAGAUGCCAAUAACAGGCUCGCCCAUGAGUAGAGCAUGGACUGCGGUGCAU | |
| | GUGACUUCGGUCACCAGGGGCAUAGCAUUGCUCACCCCUGAAUCAAGUCAUCGAGAUUUCUCUGACC | |
| | UCUGAAGUGCACUGUGGUUGCGUGGCUGGGAAUCCACGCUUGACCAUGUACUGCUUGAUAGAGUCGC | |
| | GGCUGGCCGACUCAUGGGUUAAAGUCAGUUGACAAGACAC | |
| 158 | CCACCGUUACUUCACUCCACUCCCUCGGGACUGGUUUGGAGGAGCAUAACAGGGCUUCCCAUCCCUG | Porcine- |
| | UUCACCCUCAAUACCACCCACCCUUUCCCUCAACCAUCCCUAUCCACACCCCACUGACUGAUUCCCU | Kobuvirus-XX |
| | UGGAUUUUGACCUCAGAACGCCUACUUGACCUCCCACUUGCCUUUCCCUUCUCGGAUUGCCGGUGGU | |
| | GCCUGGCGGAAAAAGCACAAGUGUGUUGCAGGCUACCAAACUCCUACCCGACAAAGGUACGUGUCCG | |
| | CGUGCUGAGUAAUGGGAUAGGAGAUGCCUACAACAGGCUCGCCCAUGAGUAGAGCAUGGACUGCGGU | |
| | GCAUGUGACUUCGGUCACCACGGGCAUAGCAUUGCUCACCCGUGAAUCAAGUCAUUGAGAUUCCUCU | |
| | GACCUCUGAAGUGCACUGUGGUUGCGUGGCUGGGAAUCCACGCUUGACCAUGUACUGCUUGAUAGAG | |
| | UCGCGGCUGGCCGACUCAUGGGUUAAAGUCAGUUGAUAAGACAC | |
| 159 | UUUGAAAAGGGGGUGGGGGGGGCCUCGGCCCCCUCACCCUCUUUUCCGGUGGCCACGCCCGGGCCAC | Caprina- |
| | CGAUACUUCCCUUCACUCCUUCGGGACUGUUGGGGAGGAACACAACAGGGCUCCCCUGUUUUCCCAU | Kobuvirus- |
| | UCCUUCCCCCUUUUCCCAACCCCAACCGCCGUAUCUGGUGGCGGCAAGACACACGGGUCUUUCCCUC | 12Q108- |
| | UAAAGCACAAUUGUGUGUGUGUCCCAGGUCCUCCUGCGUACGGUGCGGGAGUGCUCCCACCCAACUG | aichivirusC2 |
| | UUGUAAGCCUGUCCAACGCGUCGUCCUGGCAAGACUAUGACGUCGCAUGUUCCGCUGCGGAUGCCGA | |
| | CCGGGUAACCGGUUCCCCAGUGUGUGUAGUGCGAUCUUCCAGGUCCUCCUGGUUGGCGUUGUCCAGA | |
| | AACUGCUUCAGGUAAGUGGGGUGUGCCCAAUCCCUACAAAGGUUGAUUCUUUCACCACCUUAGGAAU | |
| | GCUCCGGAGGUACCCCAGCAACAGCUGGGAUCUGACCGGAGGCUAAUUGUCUACGGGUGGUGUUUCC | |
| | UUUUUCUUUUCACACAACUCUACUGCUGACAACUCACUGACUAUCCACUUGCUCUCUUGUGCCUUUC | |
| | UGCUCUGGUUCAAGUUCCUUGAUUGUUUUUGACUGCUUUUCACUGCUUUUCUUCUCACAAUCCUUGC | |
| | UCAGUUCAAAGUC | |
| 160 | UUCCCCUUCCCCCUUUUCACGGCCCGGCCGCCCGCCGGGUCGUCACCCGUUCAGACACCUACGGGUU | Rabbit- |
| | AUCUGUUCGGGCUAUAAAUAUGGGCAUUCCUCUUCCCCCUUCCCCUUUUGAAGAUGAGUGCGCAUAU | Kobuvirus |
| | UCUUGACUCCGCCUGGAUUGGCCGCCCAAGGCGUGAACAAGCAGCUAGGCCACCAUGACACUGCGGU | |
| | GGUGUCCGAACCCGCGGGUGCCUUCACGGGCACCUGUGGUAUGUAGGACUCCCACCGUGGUCUUCCC | |
| | UUUCCCCCUCAAUCUUUCCCCCUGGUUCGACUAACGGGACCAGUGCUGGAACCUGUCCGGUGAACGG | |
| | UAUAGCAGGCCCCCCCGGCAGAAACACCCGGUGCUUACCCCUUAAGGCUAGCCCCCUUCCAUGAAUU | |
| | UGGUUGGGGCAACUAGUGGGUGUACAGUUGGCGUGAACCCUCCGGUCUAGGAGUGCUCUUGCCCAAU | |
| | CCUCUGUGUGUGCCUUGCAGUAGGGACUGGCAAUCCUUCGCGUAGGUGAUCCGCUGUGCCAUGCCAU | |
| | CCUGGCGACAGGAGGCCCAGUGUGCGCAACCUACGUCCCUUCUGGGUGCUGCAUUGCAUUACCUUUG | |
| | GAGUAAGCUUGGUGUGCCGAAACCCCAGGGUUUACGUACCACUCGUGGUGUGAGGAAUGUGCCGCAG | |
| | GUACCCCAUCCUUGAGGUGGGAUCUGAGCGGUAGCUAAUUGUCUAGCACCACUUUCUUCCUUUUUUC | |
| | UUUGCUGGUCACG | |
| 161 | UACGCAUGUAUUCCACACUCAUUUCCCCCCUCCACCCUUAAGGUGGUUGUAUCCCCAUACCUUACCC | Porcine- |
| | UCCCUUCCACAAUGGACGGACAAAUGGAUUUGACCUCACGGCAAACACAUAUGGUAUGAUUUCGGAU | Sapelovirus- |
| | ACACCUUAACGGCAGUAGCGUGGCGAGCUAUGGAAAAAUCGCAAUUGUCGAUAGCCAUGUUAGUGAC | JD2011 |
| | GCGCUUCGGCGUGCUCCUUUGGUGAUUCGGCGACUGGUUACAGGAGAGUAGGCAGUGAGCUAUGGGC | |
| | AAACCUCUACAGUAUUACUUAGAGGGAAUGUGCAAUUGAGACUUGACGAGCGUCUCCUCGGAGAUGU | |
| | GGCGCAUGCUCUUGGCAUUACCAUAGUGAGCUUCCAGGUUGGGAAACCUGGACUGGGCCUAUACUAC | |
| | CUGAUAGGGUCGCGGCUGGCCGCCUGUAACUAGUAUAGUCAGUUGAAACCCCCCC | |
| 162 | UUGAAAUGGGUGUGGGGUACAUGCGUAUUACGGUACGCAUAUAUUCCACACUCAUUUCCCCCCCUCC | Porcine- |
| | ACCCUUAAGGUGGUUGUAUCCCCAUACCUUACCCUCCCUUCUAAAACAGAUGGACAAAUGGAUUUGA | Sapelovirus- |
| | ACUUAUGGCAAGUGAAUAUGGUAUGACUUUGGAUACACUUUAACGGCAGUAGCGUGGCGAGCUAUGG | A2 |
| | AAAAAUCGCAAUUGUCGAUAGCCAUGUUAGUGACGCGCUUCGGCGUGCUCCUUUGGUGAUUCGGCGA | |
| | CUGGUUACAGGAGAGUAGGCAGUGAGCUAUGGGCAAACCUCUACAGUAUUACUUAGAGGGAAUGUGC | |
| | AAUUGAGACUUGACGAGCGUCUCUUAGAGAUGUGGCGCAUGCUCUUGGCAUUACCAUAGUGAGCUUC | |
| | CAGGUUGGGAAACCUGGACUGGGCCUAUACUACCUGAUAGGGUCGCGGCUGGCCGCCUGUAACUAGU | |
| | AUAGUCAGUUGAAACCCCCC | |
| 163 | CUCACACUCAUUUCCCCCCUCGCCUGUCACAGGCAACCCCCACCCCUUCUCUUACGGGAGUUAGGGC | Stmtan- |
| | CAAGGAUCUGUUGCAUAGGCGUUGUAUCCCCUAACCUUUUACCUACCCAUCCCAAUAGGACUGGUAU | Sapelovirus- |
| | UUCGGUUUUGAUUGAGUAAUGGAUACUGAUUCUAUACCUGUUACCCAUUCAGGGGAAAAAUGGAGUU | 1 |
| | UCUUUCAUGGAUCUGACUUGAUAUGACCAAGAGUCAACACUUUGCGUGUUGGCCGUAUGGAAUGCUU | |
| | UAAGGUUUAUUCUUUGGAUUAUGACUUCAGGGUUGGCCGCCCAGGAUAAAAGGCAAUUGUGGUAAGU | |
| | GAUGUUAGUCAUUGGUGGUUGAAACCUGCCUAAGACGUCCUAGGUCUACGCUGUGCGGGCCGAAGUA | |
| | AGCUUAGGAAUAACAGGGAGUAUGCCAUUUUCUGCUUUCACCCAACACGACCGUACACGAAAGAGCU | |
| | AGAGGCACUUUGGGGCAAAGGGAAAAGCUUUGCUUAGCCCGAAUGUUCAUUUGAGUCCUUGACGAAU | |
| | GCGUCCCGUCUGUCCCGACGGUGAGGCGUAUGGCGCAUGCUCAUGGCAUUACCCAAUGGUGUAUCUG | |
| | UGAGGGGGGGGCUCCUCACACUUAGUCUAGUGCUACCUGACAGGGCCGCGGCUGGUCGUUUGUGUAU | |
| | GGUAUAACCAGUAGUAAUCCCCCAUGGAUUGCUUUAACUUCCCCUCCUCCCUUACCAAGACAUUCUC | |
| | UAAG | |
| 164 | UUUUAACUUGUUAUGACAUUCAAGGgAAAAAUGUCUUUUUCAUUAUGGGACUGACCUGUUUAUGAAC | Stmtan- |
| | AUGAGCAGCGGCACUGCUCCACGGGCUAUCCGUGUAAGAAAUAUUGAUUAUUCUUAUGGAUCAUGAU | Sapelovirus- |
| | UUCAGGGUUGGCCGCCCAGUCUAAAAGGCAAUUGUGGUAAGCUAUGUAAGUAGUUGGCUGUUGAAAG | 2 |
| | GAGCCAAGUACAUCCUAGGUCUACGCUGUGCGGGCCGAAGUAAGACUUGGAACAACUCUGAGUAGGC | |
| | AGUUUUUCUCUUUAGCCCAACACGACCGCAUACUGAAGAGCUAGAGGCACUUUGGGGCAAAGGUAAA | |
| | AGCAUUGCUUAGACCGAAUGUUCAAUGAGACCUUGACGAGUGCUGUCACAGUGUCCCCUGAUGGCAG | |
| | UAUGGCGCAUGCUCUUGGCAUUACCCAUAUGUGUAUCUAUAGGGGGGGGGCCCCCUAUACUUAGUCU | |
| | AGUGCUACCUGACAGGGCCGCGGCUGGUCGUCGGUGUGUGGUAUAACCAGUAGUAAUCCCCCAUGGA | |
| | UUGCUUUAACUCCCCCUCCUCCCUCAACAAAACUUUCUCUAAG | |
| 165 | CCGGGUAUAACCCGGAGUUUUGGGGCAGGUCCAAGCCCCACAUAGGAACAUACGAUCCACGGAUCGU | Rabovirus-C |
| | GUGUUCUUUUAUGCUUUCUAACCUUACCCUUUGUAACCAUUACGCUUUACGCCGCAUGGUGUUUGGC | |
| | GGCACCAUGACGUGGACAAGAGGUUACGCCAUUACGAUAUGUACCCUCCCCUUUUGGGGAGAGACCG | |
| | ACCAAUUAUGGUACAGUAUCCAACUGUAUUGUGGUCAAGUACUUCUGUUUCCCCGGUGAUGCGGGAU | |
| | AGGCUGUACCCACGGCCAAAACCUGCUGAUCCGUUACCCGACUCACAUCUACGAGGAGGCUAGUAAA | |
| | AGGCAUGAAGUUCAAGAGUAUGAUCCAACCAGAUCCCCACUGGUAAACUAGUGAUGAGGGUUCCCGU | |
| | UCCGAACAUGGCAACAUGUGGGUUCCCUGCGUUGGCACUAGGCCCCUUCCGAGGGGUGCUCUGAAGA | |
| | UGGAUUGUUGAUGAAGACCAAUUUGUGCAUGUGUUUAUCCUCCGGCCCUCUGA | |
| 166 | UUGAAAGACCUCGGCAUAUAUCGUUGUCACAACGGUAUAUGUCGAGAUCUUUCUCCCCACCCCCUCC | Boosepivirus- |
| | AAUUCCCUUUUCCCCCUCUUGCAACUUAGAAGUUUGUACACACAGGGCAAUAGGAUACGUGAUCCAG | A |
| | CCAGGACACGUGAGCUCAAGCACUUCUGUUUCCCCGUCCCCUUCACGUACUACGGGAAUGUUAGUAA | |
| | UUUGUGUGCACUUUAGUAAGGUUGAUCCGGGAUUAACCCCAAAUCCCAGAAACUGGUGAUGAGCGUU | |
| | ACCACCCCCGCCGGGCGACCGGAAGGUUUCGCUGCGUUGGCACCAGGGCUUCGGCACCAGAAAAAGG | |
| | UAAAGCAAAUGAAGGCGCUACUGUGCUACGAGAAGUUUCCUCCAGGCCCCUGAAUGCGGCUAAUCCU | |
| | AACCAGUGAUCCACCGGUGCAAAACCAUGUACUAGGUGGUCGUAACGCGCAAGUCGCUGGCGGAACC | |
| | GACUACUUUGGGUGUCCUGUGUUUCCAUAUUUUAUUUUAUUCAAUUUUAUGGUGACAAGAGUAAAGA | |
| | GAUACAGAUUUGCAGCC | |
| 167 | UUUUCUCCCCUCCCCCUCCAACUACCUUUUCCCCCUCUUGUAACGCUAGAAGUUUGUGCAAACCGCC | Boosepivirus- |
| | UGUAGGGUACUGCAAUCCAGCAGUGCAUAGGCUAAGCUUUUCUUGUUACCCCACCCCACAUUAUACU | B |
| | GAGGAGGAUUGUGAAAUUGUGUUAGUAUGGGUUAGUAGCGGUGACCCGGGUAACCCCAACCCAGAAA | |
| | CUCACGGAUGAGAUGAACAGGACCCCACAUGGUAACGUGUGUGUUCGUCUGCCCCGCAAGGUGAGGC | |
| | CGUGAGAGCUUUGCACGCGAAAACCUUGAAAACCCAAAAGUACCUUGAGCUCUUCGCUAUUUUGUGU | |
| | UUCCUCCAGGACCCUGAAUGCGGCUAAACCUAACCCGCGAUCCGCACGUAGCAACCCAGCUAGAGUG | |
| | UGGUCGUAAUGCGCAAGUUGCGGGCGGUACCGACUACUUUGGUGUUCCUGUGUUUCCUUUAUUUUAU | |
| | UUUGAAUUUUUAUGGUGACAACAGCUAGAAAAUAAGAGUGAAC | |
| 168 | UUAAAACUGGGUUUGGGUUGUUCCCACCCAAACCACCCACGCGGUGUUGUACACUGUUAUUCCGGUA | HRVA73 |
| | ACCUUGUACGCCAGUUUUAUAUCCCUUCCCCCCCUUGUAACUUAGAAGACAUGCGAAUCGACCAAUA | |
| | GCAGGCAAUCAACCAGAUUGUCACCGGUCAAGCACUUCUGUUUCCCCGGCUCUCGUUGAUAUGCUCC | |
| | AACAGGGCAAAAACAAUUGGAGUCGUUACCCGCAAGAUGCCUACGCAAAACCUAGUAGCAUCUUCGA | |
| | AGAUUUUUGGUUGGUCGCUCAGUUGCUACCCCAGCAAUAGACCUGGCAGAUGAGGCUAGAAAUACCC | |
| | CACUGGUGACAGUGUUCUAGCCUGCGUGGCUGCCUGCACACCCACACGGGUGUGAAGCCAAAGAUUG | |
| | GACAAGGUGUGAAGAGUCACGUGUGCUCAUCUUGAGUCCUCCGGCCCCUGAAUGCGGCUAACCUUAA | |
| | CCCCGUAGCCAUUGCUCGCAAUCCAGCGAGUAUAUGGUCGUAAUGAGUAAUUACGGGAUGGGACCGA | |
| | CUACUUUGGGUGUCCGUGUUUCACUUUUUACUUAUCAAUUUGCUUAUGGUGACAAUAUAUAUAGAUA | |
| | UAUAUUGACACC | |
| 169 | ACAUGGGCCAGCCCACCACACCCACUGGGUGUAGUAGUCUGGUUCUAUGGAACCUUUCUACGCCUCU | EV-L-1 |
| | UUUGCUUCCCUCCCCCAUUUCUCCUUCGAUUGCUCCACCUGUGAUCUUUGCAACUUAGAAGAAAUAA | |
| | UGAACCCGCACAAUAGCGGGCGCUGAGCCACAGCGUCAAUGUGCAAGCACUUCUGUUUCCCCGGAAU | |
| | GGGCCCAUAGGCUGUACCCACGGCUGAAAGGGACCGGCCCGUUACCCGCCUUGGUACUGCGAGAAUG | |
| | UUAGUAACUCCCUCGAUAGCUUUAGGCGUUACGCUCAGCCCUUUGAGCCCGAAGGGUAGUUCGGGUC | |
| | GAUGAGGCUCGUCAUUCCCCACUGGCGACAGUGUGACUUGCCUGCGUUGGCGGCCCGGGGUGGGGGG | |
| | CAACCCCCAUCCACGCCUACUGAAGGACAGGGUGUGAAGGCGCUAUUGCGCUACUAAGGAGUCCUCC | |
| | GGCCCCUGAAUGCGGCUAACCCGAACCCCGAGCCCACGGUGGUAAACCCGCCACAAGUGGGUCGUAA | |
| | UGAGUAAUUUGGGGCAGGGACCGACUACUUUGGGUGUCCGUGUUUCCUGUUUUUCCAUACGAUGGCU | |
| | GCUUAUGGUGACAACCAUAAGCAAUUGGAUUGGCCAUCCGGUGUUCAUAUUGCGAAU | |
| 170 | UUUAAAAGAGCCUGGGGGUUGUUCCCACCCCCAGGAUGCCCACUCGUGGGCAUGUGGUACUGUGGUA | EV-K-2 |
| | UCGCGGUACCACAGUACGCCUCUUUGACCGCCCUGACCCAAGAAUCUUUUCCUUUAACAGAUCUCUU | |
| | CGUAACAAGGGCCUCAGCCUGACGCAAGUGCCUCCAUUGGAGUCUCUCCAAGCCCUCCGGGGCUUGG | |
| | AGGGCGCCGACCCCCUGCCUAGGGGGAGCCCACGACACGGCUGGAGUCCAUUGGCACACCGCAGCCA | |
| | CGAUUCAAGCCAGAAUUGAAAGCGGGAAGCACUUCUGUCUCCCCGGUGUGGAUCAUACGCUGUACCC | |
| | ACGGCGAAAAGUGAAGCAUCGUUACCCGACUCGGUACUUCGAGAAGCCCAGUACAGUUGUGGAUCUC | |
| | UGCAGGGUAUACGCUCAGCGUGACCCCUACGUAGUUCCUUGAGAUGGCUGAGAGAACACCCCACGGG | |
| | CGACCGUGUCUCUCGGCGCGUGGCUCAAGGCCGGGCCUUCAGUGGCUCGGUGCCUUGCAGAGUGAAG | |
| | CCUCCGAACAGCCUAUUGAGCUACCGUUUAGCCUCCGCCCUCUUGAAUGCGGCUAAUCCUAACCAUG | |
| | GAGCGCCCGCCCACAGUCCAGUGGGUAGAGCGUCGUAACGCGCAAGUCCGUGGCGGAACCGACUACU | |
| | UUAGAGUGGCGUGUUUCCAAUUUAUCCUUUAUAAAGUUGCUUAUGGUGACACCACAAGAGAUCCACG | |
| | AUUUCUUGUUUCUUAUCACUGAGACACAAGUCAUAUUCAUCAAUCUUUAUUGCGGAAUUAACUUGGU | |
| | GCGUCCAAACACAUCAGC | |
| 171 | UUAAAAUAGCCUCAGGGUUGUUCCCACCCUGAGGGCCCACGUGGCGUAGUACUCUGGUAUCAAGGUA | EV-J-1631 |
| | CCUUUGUACGCCUAUUUUAUUUCCCUUCCCCCACAGUAACUUAGAAGCUUAUCUCAUAGUUCAACAG | |
| | UAGGGUCACUAACCAAGUGGCUCAGCGAACAAGCACUUCUGUUUCCCCGGUCCUAGUACCUGUGAAG | |
| | CUGUACCCACGGCGGAAGGGGAAAAAGAUCGUUAUCCGGCCCCCUACUUCGGAAAGCCUAGUAACAC | |
| | CAUUGAAGCAAUCGAGUGUUGCGCUCAGCACAGUAACCCCUGUGUAGCUUUGGUUGAUGAGUCUGGG | |
| | CACUCCCCACUGGCGACAGCGGCCCAGGCUGCGUUGGCGGCCAACCGACUCGGGCAACCGGGUCGGA | |
| | CGCUCGUUUGUGGACAUGGUGUGAAGAGCCUACUGAGCUAGAGGGUAGUCCUCCGGCCCCUGAAUGC | |
| | GGAUAAUCCUAACCCCGGAGCACCCACACUCAAUCCAGAGUGCAGGAUGUCGUAACGCGUAAGUCUG | |
| | GGACGGAACCGACUACUUUGGGUGUCCGUGUUUCCUGUUUUACUUACUUUGGCUGCUUAUGGUGACA | |
| | AUCUAGUGUUGUUACCAUAUAGCUAUUGGAUUGGCCAUCCGGUGUUUUGAAUUGUGUGUUUAUACUA | |
| | AUUCUUUUACAUAUCACAGACAACCAAAU | |
| 172 | UUAAAAUAGCCUCAGGGUUGUUCCCACCCUGAGGGCCCACGUGGCGUAGUACUCUGGUAUUACGGUA | EV-J-N125- |
| | CCUUUGUACGCCUAUUUUACCCCCUUCCCCUUGUAACUUAGAAGCAAAGCAAACCAGUUCAAUAGUA | J108 |
| | AGCAACACAACCCAGUGUUGUGACGAACAAGUACUUCUGUUUCCCCGGGAGGGUCUGACGGUAAGCU | |
| | GUACCCACGGCUGAAGUAUGACCUACCGUUAACCGGCUACCUACUUCGAGAAGUCUAGUAAUACCAU | |
| | UGAAGUUUUGUUGGCGUUACGCUCAACACACUACCCCGUGUGUAGUUUUGGCUGAUGAGUCACGGCA | |
| | UUCCCCACGGGCGACCGUGGCCGUGGCUGCGUUGCGGCCAACCAAGGGGCGCAAGCUCCUUGGACGU | |
| | CACUUAACAGACAUGGUGUGAAGAACCUAUUGAGCUAGGUAGUAGUCCUCCGGCCCCUGAAUGCGGC | |
| | UAAUCCUAACUCCGGAGCACAUCAGUGCAACCCAGCAUUUGGUGUGUUGUAAUACGCAAGUCUGGAG | |
| | CGGAACCGACUACUUUGGGUGUCCGUGUUUCCUGUUUUACCUUAUUUGGCUGCUUAUGGUGACAAUU | |
| | UGAUAUUGUUACCAUAUAGCUGUUGGAUUGGCCAUCCGGAUUUUUGAAAGAGACCCAAAACUUUCUU | |
| | CUCUACUUCAGAUUCAAGUGCGAAGUUUUCCUUUUCAUAUAUUACUUACUAAUUUGAAGUACCAAAG | |
| 173 | UUAAAACAGCCUGGGGGUUGUUCCCACCCCCAGGGCCCACGUGGCGCAAGUACUCUGCUUCGUUAGA | EV-1 |
| | AGCCUUGUACGCCUGUUUUCCCCUCCUUAAACAAAUUAAGAUUGCCACUUCCGAGGGGAGUAGUCCG | |
| | ACUCCGCUCCGAUACGUCGCUCCAGUGCGCUAGUACGCUUAGUACUUUCUCACGGGGAUAGUGGUAU | |
| | CCCUCCCUAGUAAUUUAGAAGACUUGAAAAACCGACCAAUAGGCACCUCGCAUCCAGCGGGGUAAAG | |
| | GUCAAGCACUUCUGUUUCCCCGGGUCGAGUAGCGAUAGACUGUGCCCACGGUCGAAGGUGAAACAAC | |
| | CCGUUAUCCGACUUUGUACUUCGGGAAGCCUAGUACCACCAAAGAUUAUGCUUGGGGUUUCGCUCAG | |
| | CACGACCCUGGUGUAGAUCAGGCCGAUGGAUCACCGCAUUCCUCACAGGCGACUGUGGCGGUGGUCG | |
| | CGUGGCAGCCUGCCGAUGGGGCAACCCAUCGGACGCCAAGCAUAUGACAGGGUGUGAAGAGCCUACU | |
| | GAGCUACAAAGUAUUCCUCCGGCCCCUGAAUGCGGCUAAUCCCAACCACGGAGCAUUUGCUACCAAA | |
| | CCAGGUAGUGGAAUGUCGUAACGGGUAACUCUGUGGCGGAACCGACUACUUUGGGUGUCCGUGUUUC | |
| | CUUUUAAUUUAUCAUUCUGUAUAUGGUGACAACUAUAGUGCUAUCUCGAUUUGCAUUACUAUUGUUG | |
| | AGAUUAAAACUUUAUUACAUUGUUGCAUUUUACCCUUUGAGUGAGUUUUCACCUGAACAGAUUAAUU | |
| | UACUCAUCCUGUUUAUAUAUUACAAGCAGAAAUACUUGCAAAG | |
| 174 | UUUAAACAGCCUGGGGGUUGUUCCCACCCCUGGGGCCCACGUGGCGCUAGUACUCUGGUACGCUAGU | EV-F1-BEV- |
| | ACCUUUGUACGCCUGUUUUUCCCCUCCCUUAAAUAAAUCAAGGUUGCCACUACUGAGGGGAGUAGUC | 261 |
| | CGACUCCGCUCCAGCAAUGCUGCACCAGUGCACUGGUACGCUAGUACCUUUUCACGGAGUAGAUGGU | |
| | AUCCCUUACCCCGGAACCUAGAAGAUUGCACACAAACCGACCAAUAGGCGCACCGCAUCCAGCCGUG | |
| | CAGCGGUCAAGCACUUCUGUCUCCCCGGUCUGUAAAGAUCGUUAUCCGCCCGACCCACUACGAAAAG | |
| | CCUAGUAACUGGCCAAGUGAACGCGAAGUUGCGCUCCGCCACAACCCCAGUGGUAGCUCUGGAAGAU | |
| | GGGGCUCGCACCACCCCCGUGGUAACACGGUUGCCUGCCCGCGUGUGCUUCCGGGUUCGGUCUCGUG | |
| | CCGUUCACUUCAACUUCACGCAACCAGCCAAGAGCCUAUUGUGCUGGGACGGUUUUCCUCCGGGGCC | |
| | GUGAAUGCUGCUAAUCCCAACCUCCGAGCGUGUGCGCACAAUCCAGUGUUGCUACGUCGUAACGCGU | |
| | AAGUUGGAGGCGGAACAGACUACUUUCGGUACCCCGUGUUUCCUCUCAUUUUAUUUAAUAUUUUAUG | |
| | GUGACAAUUGUUGAGAUUUGCGCUCUUGCAACGUUGCCAUUGAAUAUUGGCUUAUACUAUUUGGUUG | |
| | CCUUUUACAAAACCUCUGAUAUACCCAGUUCUUACAUUGAUCUGCUUGUUUUUCUCAAUUUGAAGUA | |
| | UAGACUACAAAUAGCAAA | |
| 175 | UUAAAACAGCCUGUGGGUUGUUCCCACCCCAGAGGCCCACGUGGCGGCCAGUACUCUGGUAUCACGG | EV-D94 |
| | UACCUUUGUACGCCUGUUUUAUAUCCCCUUCCCCCGCAACUUAGAAGAAAACAAAUCAAGUUCACUA | |
| | GGAGGGGGUACAAACCAGUACCACCACGAACAAGCACUUCUGUUUCCCCGGUGAUGUCGUAUAGACU | |
| | GUAACCACGGUUGAAAACGAUUGAUCCGUUAUCCGCUCUUGUACUUCGAAAAGCCCAGUAUCACCUU | |
| | GGAAUCUUCGAUGCGUUGCGCUCAGCACUCAACCCCAGAGUGUAGCUUAGGUCGAUGAGUCUGGACA | |
| | CUCCUCACCGGCGACGGUGGUCCAGGCUGCGUUGGCGGCCUACCUGUGGUCCAAAGCCACAGGACGC | |
| | UAGUUGUGAACAAGGUGUGAAGAGCCUAUUGAGCUACAAGAGAAUCCUCCGGCCCCUGAAUGCGGCU | |
| | AAUCCUAACCACGGAGCAAGGGUACACAAACCAGUGUAUAUCUUGUCGUAACGCGCAAGUCUGUGGC | |
| | GGAACCGACUACUUUGGGUGUCCGUGUUUCCUUUUGUUUUUAUCAUGGCUGCUUAUGGUGACAAUCU | |
| | AAGAUUGUUAUCAUAUAGCUGUUGGAUUGGCCAUCCGGUAAUUUAUUGAGAUUUGAGCAUUUGCUUG | |
| 176 | UUUCUUCAACAAUUUCACCUAUUCAUUGCAUUUCAGCAGUCAAA | |
| | UUAAAACAGCUCUGGGGUUGUUCCCACCCCAGAGGCCCACGUGGCGGCUAGUACACUGGUAUCACGG | PV3 |
| | UACCUUUGUACGCCUGUUUUAUACUCCCUCCCCCGCAACUUAGAAGCAUACAAUUCAAGCUCAAUAG | |
| | GAGGGGGUGCAAGCCAGCGCCUCCGUGGGCAAGCACUACUGUUUCCCCGGUGAGGCCGCAUAGACUG | |
| | UUCCCACGGUUGAAAGUGGCCGAUCCGUUAUCCGCUCAUGUACUUCGAGAAGCCUAGUAUCGCUCUG | |
| | GAAUCUUCGACGCGUUGCGCUCAGCACUCAACCCCGGAGUGUAGCUUGGGCCGAUGAGUCUGGACAG | |
| | UCCCCACUGGCGACAGUGGUCCAGGCUGCGCUGGCGGCCCACCUGUGGCCCAAAGCCACGGGACGCU | |
| | AGUUGUGAACAGGGUGUGAAGAGCCUAUUGAGCUACAUGAGAGUCCUCCGGCCCCUGAAUGCGGCUA | |
| | AUCCUAACCAUGGAGCAGGCAGCUGCAACCCAGCAGCCAGCCUGUCGUAACGCGCAAGUCCGUGGCG | |
| | GAACCGACUACUUUGGGUGUCCGUGUUUCCUUUUAUUCUUGAAUGGCUGCUUAUGGUGACAAUCAUA | |
| | GAUUGUUAUCAUAAAGCGAGUUGGAUUGGCCAUCCAGUGUGAAUCAGAUUAAUUACUCCCUUGUUUG | |
| | UUGGAUCCACUCCCGAAACGUUUUACUCCUUAACUUAUUGAAAUUGUUUGAAGACAGGAUUUCAGUG | |
| | UCACA | |
| 177 | UUAAAACAGCUCUGGGGUUGUUCCCACCCCAGAGGCCCACGUGGCGGCUAGUACUCUGGUAUCACGG | EV-C102 |
| | UACCUUUGUACGCCUGUUUUACAUCCCCUCCCCCACGUAACUUUAGAAGCAAUUCAACAAGUUCAAU | |
| | AGAGGGGGUACAAACCAGUAUCACCACGAACAAGCACUUCUGUUUCCCCGGUGAUUUUACAUAAGCU | |
| | GUGCCCACGGCUGAAAGUGAAUGAUCCGUUACCCGCUCGAGUACUUCGAAAAGCCUAGUAUCGCUUU | |
| | GGGAUCUUCGACGCGUUGCGCUCAGCACUCUACCCCGAGUGUAGCUUAGGCUGAUGAGUCUGGGCAU | |
| | UCCCCAUCGGCGACGAUGGCCCAGGCUGCGUUGGCGGCCUACCCAUGGCUAACGCCAUGGGACGCUA | |
| | GUUGUGAACAAGGUGUGAAGAGCCUAUUGAGCUACUCGAGAGUCCUCCGGCCCCUGAAUGCGGCUAA | |
| | UCCCAACCACGGAUCAGGUGCCUCCAACCCAGGAGGUGGCCUGUCGUAACGCGCAAGUCUGUGGCGG | |
| | AACCGACUACUUUGGGUGUCCGUGUUUCCUUUUAUCUUUUAAAUGGCUGCUUAUGGUGACAAUCAUA | |
| | GAUUGUUAUCAUAAAGCGAAUUGGAUUGGCCAUCCGGUGAAAUACAAACACAUUAUUUACUUGUUUG | |
| | UUGGAUUUACUCCGCUCACACAGCUUACUCCUAAGAUAAUAUUUAUUGUAUUGCUGGUAAGGAGACA | |
| | CUAUUAUA | |
| 178 | UUAAAACAGCCUGUGGGUUGAUCCCACCCACAGGGCCCACUGGGCGCCAGCACACUGGUAUCGUGGU | EV-30 |
| | ACCUUUGUGCGCCUGUUUUAUACCCCCUACCCCCACAUGUACUCUAGAAGCAAGGCAAACCUGACCA | |
| | AUAGUAGGUGUGGCACACCAGCCGCAUUUUGGUCAAGCACUUCUGUUUCCCCGGACCGAGUAUCAAU | |
| | AAGCUGCUCACGCGGCUGAAGGAGAAACCGUUCGUUACCCGACCAGCUACUUCGAGAAACCUAGUAA | |
| | CACUAUGAACGUUGCGGAGUGUUUCGUUCAGCACUUCCCCCGUGUAGAUCAGGUCGAUGAGUCACCG | |
| | CAUUCCUCACGGGUGACCGUGGCGGUGGCUGCGUUGGCGGCCUGCCUACGGGUUCGCCCGUAGGACG | |
| | CUCUAAUACCGACAUGGUGUGAAGAGUCCAUUGAGCUAGCUGGUAGUCCUCCGGCCCCUGAAUGCGG | |
| | CUAAUCCUAACUGCGGAGCAGGUGCUCACAGACCAGUGAGUAGCCUGUCGUAACGGGCAACUCUGCA | |
| | GCGGAACCGACUACUUUGGGUGUUUUUCCUUUUUUCUUCUCUUAUAUUGGCUGCUUAUGGUGACAAU | |
| | UAAAGAAUUGUUACCAUAUAGCUAUUGGAUUGGCCAUCCGGUGACGAGCAGAGCCAUUGUUUACCUC | |
| | UUUGUUGGAUUUGUACCUUUGAACCACAAAGUCUUGAAUACCAUUCAUCUCAUUUUAAAGUUCAACU | |
| | CAGCUAAAAGAAA | |
| 179 | UUAAAAUAGCCUGUGGGUUGCUCCCACCCACAGGGCCCAAGUGGCGUAGUACUUGGUAUUCCGGUAC | SA5- |
| | CUUUGUACACCUAUUUACAAACCCUACCCCUUGUAACCUUAGAAGCAAUUAUUUAACCGCUCACUAG | enterovirusB114 |
| | GGGGUGUGCUAUCCAAGCACAUCAAGAGCAAGCACUUCUGUCUCCCCGGGAGGGGCUAAUGGUACGC | |
| | UGUGCCCACGGCGGAAAUGAGCCCUACCGUUAACCGGCAGUCUACUUCGGGAAGCCCAGUAACUACA | |
| | UUGAAACUUUGAGGCGUUACACUCAGCACAUAACCCCAAUGUGUAGUUCUGGUCGAUGAGCCUUGGC | |
| | AUCCCCCACAGGCGACUGUGGCCAAGGCUGCGUUGGCGGCCAGCCUGCGGACCAAAAGUCCGUAGGA | |
| | CGCCUAAUUGUGGACAUGGUGUGAAGAGCCUACUGAGCUAGACUGUAGUCCUCCGGCCCCUGAAUGC | |
| | GGCUAAUCCUAACCCUGGAGCAUCCGCGUGCAACCCAGUACGUAGGGUGUCGUAAUGCGUAAGUCUG | |
| | GGAUGGAACCGACUACUUUGGGUGUCCGUGUUUCUUGUUUUUCAUACUGGGUCGCUUAUGGUUACAA | |
| | CUAAUUGUUGUAAUCAUUGGCAGUGCGCGCUGACCACGCGAUUAUUGAUAUUUCCAUUUGUUGGAUA | |
| | CUCCAAUAGUGUCAACUCAUAUACACAACUUUUACCACUGAUCAAGAUAAAA | |
| 180 | UUAAAACAGCCUGUGGGUUGUUCCCACCCACAGGGCCCACUGGGCGCUAGCACUCUGGUUCUCCGGA | EV-A114 |
| | ACCUUUGUGCGCCUGUUUUGAAACCCCCUCCCCCAACUCGAAACGUAGAAGUAAUGUACACUACUGA | |
| | UCAGUAGCAGGCGUGGCGCACCAGCCAUGUCUCGAUCAAGCACUUCUGUUUCCCCGGACUGAGUAUC | |
| | AAUAGACUGCUCACGCGGUUGAAGGUGAAAACGUCCGUUACCCGGCUAACUACUUCGAGAAACCUAG | |
| | UAGCACCAUAGAAACUGCAGAGUGUUUCGCUCAGCACUUCCCCCGUGUAGAUCAGGUCGAUGAGUCA | |
| | CUGCAAUCCCCACGGGUGACCGUGGCAGUGGCUGCGUUGGCGGCCUGCCUAUGGGGCAACCCAUAGG | |
| | ACGCUCUAAGGUGGACAUGGUGUGAAGAGUCUAUUGAGCUAGUUAGUAGUCCUCCGGCCCCUGAAUG | |
| | CGGCUAAUCCUAACUGUGGAGCGCAUACUCCCAAACCAGGGAGCAGUGCGUCGUAACGGGCAACUCC | |
| | GCAGCGGAACCGACUACUUUGGGUGUCCGUGUUUCCUUUUAUUCCUAUACUGGCUGCUUAUGGUGAC | |
| | AAUUGAGAGAUUGUUACCAUAUAGCUAUUGGAUUGGCCAUCCAGUGUGUAAUAGAGCAAUCAUUUAC | |
| | CAAUUUGUUGGAUUUACUCCAUUAACCCACACGUCUCUCAACACACUACAUUUCAUCUUACUACUGA | |
| | ACACUAGAAA | |
| 181 | CUCCCCCCCCUUCCCCUUCCCGAGUAGGAGAUUGGCAUGUAUGCUCUACAUGCCCGAUUCUCUCUUG | Burpengary- |
| | CUCACUCUCUUAAAUCCUGGUGGCGGUCUCGGAUUAAACAUUUAUGUCGUAUCUGGGAUCGUCUUAC | virus |
| | UUGGUGGUAAUUCCUCUGUUGCCUAGGGACCUCCGGACUGCCGGAUUAAAGGUCUCAACAGAGGGCA | |
| | AUGUACAAGGAAGUCAUUAUACGCUAAUUAAGUAUUUGAUGAAUGACUAGUGUGACAGGGCUGAGGA | |
| | ACUCCCCCCGGGUAACCGGUGCCUCAGCGUCCGAAAGACACGUGGAUAGGAUCCACCCUGUUAUACC | |
| | CAGCACGAUGUAAUAGUCAAAUACCUCUGAUUUGUGUAGGAUGUAUAAAUUGUGCAUUGUAAAUUUU | |
| | GGGCGUAGAGAUGCUCCGAAGGUACCCCGUUUUACGGGAUCUGAUCGGAGGCUAAUUACCCAAUGCG | |
| | CCCUAAAUAACUUCAUAUAAUUUCUUUUUCUUUAUUCAAA | |
| 182 | AUGGGGAAGGGUAUGACGUGCCCCUUCCUUCUUCGGAGAACUCGCUCUAGUGGUCUUUCCACUUCUG | GFTV |
| | GAAAAGAGUGAGUGCACGUGAUCAGGACCGUCGAAGACGACAAAUACCUGGUGCUCUAUCUCAUAGA | |
| | CGUUUCACAGCUGUAGCGACCCCUCAGUAGCAGCGGAAGCCCCCUCCUGGUGACAGGAGCCUCUGCG | |
| | GCCAAAAGCCACGUGGAUAAGAUCCACUGCUGAGGGCGGUGCGACCCUAGCACCCUGUGAUGCAUAC | |
| | UAGUUGUAGCGUGCCGGACUAUUGGUCUGUCAUAAGACACCUGAUAGAGAGACCAAGAAUGUCCUGG | |
| | AGGUACCCCGCGUGCGGGAUCUGACCAGGAGACCAUUGCCCAAUGCUUUACAACGGGUCUAUGGUUU | |
| | AAAAACUGUCGCAGUCUCUCCAAACCAAGUGGUCUUGGUUUUCAAUUACUUUGAAUAUUUCACU | |
| 183 | AAGACCAUAUAUCUUAUCCCUUGAAUGCUGGCUAAUCAGAGGAAAGUCAGCAUUUUCCGGCCCAGGC | SAFV-V13C |
| | UAAGAGAAACAUAUAAGUUAGAUUUGAUUUAAAUCCCUUGACGAAUUCGAAACGAGAUGUUCUCCCU | |
| | CCCUUGCCGCUUGUUCACACCCACUUAUUUAAUUCGGCCUUUUGUGACAAGCCCCUCGGUGAAAGAA | |
| | CCUCUCUCUUUUCGACGUGGUUGGAAUUGCCAUCAUUUCCGACGAAAGUGCUAUCAUGCCUCCCCGA | |
| | UUAUGUGAUGUUUUCUGCCCUGCUGGGCGGAGCAUUCUCGGGUUGAGAAACCUUGAAUCUUUUUCUU | |
| | UGGAACCUUGGUUCCCCCGGUCUAAGCCGCUUGGAAUAUGACAGGGUUAUUUUCUUGAUCUUAUUUC | |
| | UACUUUUGCGGGUUCUAUCCGUAAAAAGGGUACGUGCUGCCCCUUCCUUCUCUGGAGAAUUCACACG | |
| | GCGGUCUUUCCGUCUCUCAACAAGUGUGAAUGCAGCAUGCCGGAAACGGUGAAGAAAACAGUUUUCU | |
| | GUGGAAAUUUAGAGUGCACAUCGAAACAGCUGUAGCGACCUCACAGUAGCAGCGGACUCCCCUCUUG | |
| | GCGACAAGAGCCUCUGCGGCCAAAAGCCCCGUGGAUAAGAUCCACUGCUGUGAGCGGUGCAACCCCA | |
| | GCACCCUGGUUCGAUGAUCAUUCUCUAUGGAACCAGAAAAUGGUUUUCUCAAGCCCUCCGGUAGAGA | |
| | AGCCAAGAAUGUCCUGAAGGUACCCCGCGUGCGGGAUCUGAUCAGGAGACCAAUUGGCGGUGCUUUA | |
| | CACUGUCACUUUGGUUUAAAAAUUGUCACAGCUUCUCCAAACCAAGUGGUCUUGGUUUUCCAAUUUU | |
| | GUUGAAUGGCAAU | |
| 184 | GGGGAUCUCCCCCGCGGCAGCUGGUUACAGCUGUCGCGGACGGAGAUCAUCCCCCGGCCACCCCCUU | SAV-P-113 |
| | UCGACGCGGGUACUGCGAUAGUGCCACCCCAGUCUUUCCUACUCCCGACUCCCGACUCUAACCCAGG | |
| | UUCCUUGGAACAGGAACACCAAUAUACUCAUCCCCUGGAUGCUGACUAAUCAGAGGAACGUCAGCAU | |
| | UUUCCGGCCCAGGCUAAGAGAAGUAGAUAAGUUAGAUUCCAAAUUGAUUUAUCAUCCCCUUGACGAA | |
| | UUCGCGUUGGAAAUGCACCUCUCACUUGCCGCUCUUCACACCCAUUAACUUGAUUCGGCCUCUGUGU | |
| | UGAGCCCCUUGUUGAAGUGCUUCCCUCCAUCGUGACGUGGUUGGAGAUCUAAGUCAACCGACUCCGA | |
| | CGAAACUACCAUCAUGCCUCCCCGAUUAUGUGAUGCUUUCUGCCCUGCUGGGUGGAGCAUCCUCGGG | |
| | UUGAGAAAACCUUCUUCCUUUUUCCUUGGACCCCGGUCCCCCGGUCUAAGCCGCUUGGAAUAAGACA | |
| | GGGUUAUCUUCACCUCUUCCUUCUUCUACUUCAUAGUGUUCUAUACUAUGAAAGGGUAUGUGUCGCC | |
| | CCUUCCUUCUUUGGAGAACACGCGCGGCGGUCUUUCCGUCUCUCGAAAAGCGCGUGUGCGACAUGCA | |
| | GAGAACCGUGAAGAAAGCAGUUUGCGGACUAGCUUUAGUGCCCACAAGAAAACAGCUGUAGCGACCA | |
| | CACAAAGGCAGCGGACCCCCCCUCCUGGCAACAGGAGCCUCUGCGGCCAAAAGCCACGUGGAUAAGA | |
| | UCCACCUUUGUGUGCGGCACAACCCCAGUGCCCUGGUUUCUUGGUGACACUUCAGUGAAAACGCAAA | |
| | UGGCGAUCUGAAGCGCCUCUGUAGGAAAGCCAAGAAUGUCCAGGAGGUACCCCUUCCCUCGGGAAGG | |
| | GAUCUGACCUGGAGACACAUCACAUGUGCUUUACACCUGUGCUUGUGUUUAAAAAUUGUCACAGCUU | |
| | UCCCAAACCAAGUGGUCUUGGUUUUCACUCUUUAAACUGAUUUCACU | |
| 185 | GACCCUAACCCAGGUUCCUCGGAUCAGGAACACAAAUUUACUCAUCCCCUGGAUGCUGACUAAUCAG | VHEV- |
| | AGGAAAGUCAGCAUUUUCCGGCCCAGGCUAAGAGAAGUAGAUAAGUUAGAUUCCAAAUUGACCUACC | cardiovirusB2 |
| | AUCCCCAUGACGAAUUCACAUUGGAAAUACACCUCUCACUUGCUGCUCUUCACACCCAUCAAUUAAU | |
| | UCGGCCCACUGUGUUGAGCCCCUCGUUGAAGUGUUUCCCUCCUUCGUGACGUGGUUGGAGAUCCAAG | |
| | UCAUCCGACUCCGACGAAACUACCAUCAUGUCUCCCCGAUUAUGUGAUGCUUUCUGCCCUGCUGGGU | |
| | GGAGCAUCCUCGGGUUGAGAAUUCCUUCUUCCUUUCUCCUUGGACCUCGGUCCCCCGGUCUAAGCCG | |
| | CUCGGAAUAUGACAGGGUUAUUUUCACCUCUUCUCUCUUCUACUUCAUAGUGUUCUAUACUAUGAAA | |
| | GGGUAUGUGUCGCCCCUUCCUUCUUGGAGAACGUGCGUGGCGGUCUUUCCGUCUCUCGAAAAACGUG | |
| | CGUGCGACAUGCAGAGUAACGCAAAGAAAGCAGUUCUUGGUCUAGCUCUGGUGCCCACAAGAAAACA | |
| | GCUGUAGCGACCACACAAAGGCAGCGGAAACCCCCUCCUGGUAACAGGAGCCUCUGCGGCCAAAAGC | |
| | CACGUGGAUAAGAUCCACCUUUGUGUGCGGUGCAACCCCAGCACCCUGGUUUCUUGGUGACACCUUA | |
| | GUGAACCCUCGAAUGGCAAUCUCAAGCGCCUCUGUAGGAAAGCCAAGAAUGUCCAGGAGGUACCCCU | |
| | UCCUCAUGGAGGGAUCUGACCUGGAGACACAUCACACGUGCUAUACACUUGUGCUUGUGUUUAAAAA | |
| | UUGUCACAGCUUUCCCAAACCAAGUGGUCUUGGUUUUCCCUUAACUUCGAAAAGUCACU | |
| 186 | CACCCCCUUUGGACGCGGGGCCUGCGAUAGUGCCACCCCCCGUCCUCCCUAUUCCAACUACCCGACC | TRV-NGS910- |
| | CUAACCCAGGGUCCAGGAAACUGGAUCAAUACAAGUCAUCCCCUGAAUGCUGGCUAAUCAGAGGAAA | cardiovirusB4 |
| | GUCAGCAUUUUCCGGCCCAGGCUAAGAGAAACACAAUAAGUUAGAAUCUAAAUUAAUCCCCUUGACG | |
| | AAUUCGCAACGAUAAGUCCUCCCUCCCUUGCCGCUCGUUCACACCCAGAACUAACAAUUCGGCCCCU | |
| | UGUGACGAGCCCCUUGGUGAAAGGACCUCUUUCAAUGCGACGUGGUUGGAGAUUAAACCGACUCCGA | |
| | CGAAAGUGCUAUCAUGCCUCCCCGAUUAUGUGAUGUUUUCUGCCCUGCUGGGCGGAGCAUUCUCGGG | |
| | UUGAUACACCUUGAAUCCUUCAUCCUUGGACCUCAGGUCCCCCGGUCUAAGCCGCUUGGAAUACGAC | |
| | AGGGUUAUUUUCCAAUCUUCUCCUUUCUACUUUCAUGAGUCCUAUUCAUGAAAAGGGUCUGUGCUGC | |
| | CCCUUCCUUCUUGGAGAAUCUGCGCGGCGGUCUUUCCGUCUCUCGAAAAGCGCAGAUGCAGCAUGCU | |
| | GGAACCGGUGAAGAAAACAGUUCUUUGUGGAAACUUAGAGCAGACAUCGAAACAGCUGUAGUGACCU | |
| | CACAGUAGCAGCGGAACCCCCUCCUGGUAACAGGAGCCUCUGCGGCCAAAAGCCCCGUGGAUAAGAU | |
| | CCACUGCUGUGAGCGGUGCAACCCCAGCACCCUGGUUCGAUGGUUGUUCUCUGUGGAACCAGAGAAU | |
| | GGUCUUUCUCAAGCCCUCCAGUAGAGAAGCCAAGAAUGUCCUGAAGGUACCCCGCAUGCGGGAUCUG | |
| | AUCAGGAGACCAAUCGUCAGUGCUUUACACUGGCGCUUUGGUUUAAAAACUGUCACAGCUUCUCCAA | |
| | ACCAAGUGGUCUUGGUUUUCACUUUUAUCAAACUGUUUC | |
| 187 | AAAUACUGGUCGAAACCGCUUGGGAUAAGACCGGGGUUUGUUAAUGUCUCAAUGUUAUUCUCCACCC | EMCV2- |
| | AAUUGACGUCUUUUGUCAAUUGGAGGGCAGUGAAACCUUGCCCUUGCUUCUUGCAGAGGAUUCCCAG | RD1338- |
| | UGGUCUUUCCGCUCUCGACAAGGGAAUUCAUGAUCCACCAAAAGUUGUGAAGAGAGCAGGUCCCAUG | cardiovirusA2 |
| | GAAGCUUUCUGACGACUGAUGAUGACUGUAGCGACCCUUUGCAGGCAGCGGACCCCCCCACCUGGUG | |
| | ACAGGUGCCUCUGCGGCCAAAAGCCACGUGUUUAACAGACACCUGCAAAGGCGGCACAACCCCAGUG | |
| | CCUCAUCAAAAGUCUGAUGACUGUGGAAAUAGUCAACCGGCUUUUCUUAAGCAAAUUUGGUGUCGGG | |
| | GCUGAAGGAUGCCCGGAAGGUACCACACUGGUUGUGAUCUGAUCCGGGGCCACAGUACAUGUGCUUU | |
| | ACACAUGUAGCUGCGGUUAAAAAACGUCUAGGCCCCCCGAACCACGGGGACGUGGUUUUCCUUUGAA | |
| | AACCACGAUUACAAU | |
| 188 | UUGAAAGCCGGGGGUGGGAGAUCCGGAUUGCCAGUCUGCUCGAUAUCGCAGGCUGGGUCCGUGACUA | EMCV1-JZ1203 |
| | CCCACUCCCCCUUUCAACGUGAAGGCUACGAUAGUGCCAGGGCGGGUACUGCCGUAAGUGCCACCCC | |
| | AAAACAACAACAAACCCCCCCUAACAUUACUGGCCGACGCCGCUUGGAAUAAGGCCGGUGUGCGUUU | |
| | GUCUAUAUGUUAUUUCCCACCACAUUGCCGUCUUUUGGCAAUGUGUGGGCCCGGAAACCUGGCCCUG | |
| | UCUUCUUGACGAGCAUUCCUAGGGGUCUUUCCCCUCUCGCCAAAGGAAUGCAAGGUCUGUUGAAUGU | |
| | CGUGAAGGAAGCAGUUCCUCUGGAAGCUUCUUGAAGACAAACAACGUCUGUAGCGGCCCUUUGCAGG | |
| | CAGCGGAACCCCCCACCUGGCGACAGGUGCCUCUGCGGCCAAAAGCCACGUGUAUAAGAUACACCUG | |
| | CAAAGGCGGCACAACCCCAGUGCCACGUUGUGAGUUGGAUAGUUGUGGAAAGAGUCAAAUGGCUCUC | |
| | CUCAAGCGUAUUCAACAAGGGGCUGAAGGAUGCCCAGAAGGUACCCCAUUGUAUGGGAUCUGAUCUG | |
| | GGGCCUCGGUGCACAUGCUUUACAUGUGUUUAGUCGAGGUUAAAAAACGUCUAGGCCCCCCGAACCA | |
| | CGGGGACGUGGUUUUCCUUUGAAAAACACGAUGAUAAU | |
| 189 | AUGUGGUCGAAGCCACUUGGAAUAAGACCGGCGUGCGCUUGUCUAUAUGUUACUUCCACCACAUUGC | EMCVI-AnrB- |
| | CGUCUUUUGGCAAUGUGAGGGCCCGGAACCUGGCCCUGUCUUCUUGACGAACAUUCCUAGGGGACUU | 3741 |
| | UCCCCUCUCGCCAAAGGAAUGUAAGGUCUGUUGAAUGUCGUGAAGGAAGCAGUUCCUCUGGAAGCUU | |
| | CUUGAAGACAAACAGCGUCUGUAGCGACCCUUUGCAGGCAGCGGAACCCCCCACCUGGUAACAGGUG | |
| | CCUCUGCGGCCGAAAGCCACGUGUAUAAGAUACACCUGCAAAGGCGGCACAACCCCAGUGCCACGUU | |
| | GUGCGUUGGAUAGUUGUGGAAAGAGUCAAAUGGCUUUCCCCAAGCGUAUUCAACAAGGGGCUGAAGG | |
| | AUGCCCAGAAGGUACCCCACUGGUUGGGAUCUGAUCUGGGGCCUCGGUGCAGGUGCUUUACACCUGU | |
| | UGAGUCGAGGUUAAAAAACGUCUAGGCCCCCCGAACCACGGGGACGUGGUUUUCCUAGAAAACCACU | |
| | AUGACAAU | |
| 190 | CUCUCUCUUUGACCCUGGCUACUGGGGUUCAGUAGCAUCUUUCUUUUUCUUACUAUACUUUACUUUC | Malagasiviru |
| | GACUUCUUUCUUUACUUAUUCUUUACUUUUCUAUCUUCUUUUAUAUCUUAACUUUACUUAUUCAAAC | S-B- |
| | UUUUCUAUUUCUUUCUUUAUUUUCUUAUGUAACUCUUCUUUUUAAGUUUUAUUUUGCCUACUUGUGA | Lesavirus-2 |
| | GCUUAUGCGGGACCACUGUCUUAGACAACCCCACAUUUGUCAUGAGUAAGUACACGCAACCAUUACG | |
| | AUUACUUUUUAACCGUCUGACCUUUUGAUAACAACUGAAGUUAGGCGUGAAACAUGCAUUUAUACCA | |
| | AAGUAGCCCCGCAUUUCCCCACUACGGUGGGGGGGCUACCCUACUGGCUUUGGAACUGUAGCCAUUA | |
| | UGUGUUGCCUGGCUUUCAGGAUCUCACAACACAACAGUUCUCUCACAAUGGAAUAUGGGUGAGAUUG | |
| | CAGUGACAUGAACAAGUAUCUAGUAGUACAUAGACUCAAGCCUAGUUGCCUGCGGAACAACAUGUGG | |
| | UAACACAUGCCCCAGGGUCCAAAAGACAAGGGUUAACAGCCCCUUCUAGGUGUCUGUGUGUGAAGAA | |
| | UACUUUAGUAGUGUUGUUAUGAUCUCACCUGUUAGUACAGAAUGAGUAUGGCUUGGUGAAGGAUGUC | |
| | CUACAGGUACCCAUUAUAUGGAUCUGAGUAGGAGACCACUAGUGGUGGCUUUACCGCCAGGUGAGUG | |
| | GUUUAAAAAGCGUCUAGCCAAGCCAACAGCACUAGGGAUAGUGCUUUCUAUAUUUUAUAUUUUCAGU | |
| | GUAUAUGGUGACAA | |
| 191 | CCCCUUUACGUAACUGCAACUUAAAGAGUACCCUACUGCAUUGGAUGUGUGGUAAACUUUUACGCAC | Tottorivirus |
| | ACAUUUGUAGUAGUGUUAGUUAUGUUCUACCUAAUGAGUAUGCAUGCACCCGUCGAAACACGCUUGU | |
| | GAUAAGAUAGGUGAGUCCAUGUGACUAAUCUCAUUAAGAUAAAUAAGCACCCUACAACGCACGGCAC | |
| | GCUCGUGUCUUCCGUGCGGGGCCGGGACAACAGCGGCCUAAAUCUUCUAGGUGACCACCAUGCUUUU | |
| | GGGACUAUGGCACCACUGUGGACGUGAGUACCUGGCAGUAAGUCUGUGAAAAGAUGGAAGGUGUCCC | |
| | AAGCUAUGGGGCGUAUGCAUAUAGCCUGCGGAACAAACAACGGCGACGUUGUCCCCAGGGCCCAAAA | |
| | GGCACGUGGAUAAGAUCCACCUAUAUGUUUACCCCAUAGUGUAAGUCACUGGAAGUCCUAGUAAUGG | |
| | AUGUCUGGAGUAAGGCUCACGGGGUAGGGCGAAGGAUGCCCAGAAGGUACCCGUAGGUAACCUUAAG | |
| | AGACUAUGGAUCUGAUCUGGGGACCGGAUGGCGCCAUCACCAUGACGUGGAGGCCGGUUUAAAAAAC | |
| | GUCUAAGCCCGACCAACAACCUAGGGGACUAGGUUUUCCUUUUUUAUUCAUGUAUGACGUU | |
| 192 | UUUCGACCGCCUUAUGGCAGGCGGGCUUGUGGACGGCUUCGGCCCACCCACAGCAAGAAUGCCAUCA | Salivirus-A- |
| | UCUGUCCUCACCCCCAUUUCUCCCCUCCUUCCCCUGCAACCAUUACGCUUACUCGCAUGUGCAUUGA | 02394-A105- |
| | GUGGUGCACGUGUUGAACAAACAGCUACACUCACGUGGGGGCGGGUUUUCCCGCCCUUCGGCCUCUC | 675 |
| | GCGAGGCCCACCCUUCCCCUUCCUCCCAUAACUACAGUGCUUUGGUAGGUAAGCAUCCUGAUCCCCC | |
| | GCGGAAGCUGCUCGCGUGGCAACUGUGGGGACCCAGACAGGUUAUCAAAGGCACCCGGUCUUUCCGC | |
| | CUCCAGGAGUAUCCCUGCUAGUGAAUUCUAGUGGGGCUCUGCUUGGUGCCAACCUCCCCCAAAUGCG | |
| | CGCUGCGGGAGUGCUCUUCCCCAACUCACCCUAGUAUCCUCUCAUGUGUGUGCUUGGUCAGCAUAUC | |
| | UGAGACGAUGUUCCGCUGUCCCAGACCAGUCCAGCAAUGGACGGGCCAGUGUGCGUAGUCGUCUUCC | |
| | GGCUUGUCCGGCGCAUGUUUGGUGAACCGGUGGGGUAAGGUUGGUGUGCCCAACGCCCGUACUUUGG | |
| | UGACAACUCAAGACCACCCAGGAAUGCCAGGGAGGUACCCCGCCUCACGGCGGGAUCUGACCCUGGG | |
| | CUAAUUGUCUACGGUGGUUCUUCUUGCUUCCAUUUCUUUCUUCUGUUC | |
| 193 | UUAAAACAGCCUGUGGGUUGUACCCACCCACAGGGCCCACUGGGCGCUAGCACACUGGUAUUACGGU | CVA3-A115- |
| | ACCUUUGUGCGCCUGUUUUAUACCCCCCCCAACCUCGAAACUUAGAAGUAAAGCAAACCCGAUCAAU | 675 |
| | AGCAGGUGCGGCGCACCAGUCGCAUCUUGAUCAAGCACUUCUGUAACCCCGGACCGAGUAUCAAUAG | |
| | ACUGCUCACGCGGUUGAAGGAGAAAACGUUCGUUACCCGGCUAACUACUUCGAGAAACCCAGUAGCA | |
| | UCAUGAAAGUUGCAGAGUGUUUCGCUCAGCACUACCCCCGUGUAGAUCAGGCCGAUGAGUCACCGCA | |
| | CUUCCCCACGGGCGACCGUGGCGGUGGCUGCGUUGGCGGCCUGCCUAUGGGGCAACCCAUAGGACGC | |
| | UCUAAUACGGACAUGGUGCGAAGAGUCUAUUGAGCUAGUUAGUAGUCCUCCGGCCCCUGAAUGCGGC | |
| | UAAUCCUAACUGCGGAGCACAUACCCUUAAUCCAAAGGGCAGUGUGUCGUAACGGGUAACUCUGCAG | |
| | CGGAACCGACUACUUUGGGUGUCCGUGUUUCCUUUUAAUUUUUACUGGCUGCUUAUGGUGACAAUUG | |
| | AGGAAUUGUUGCCAUAUAGCUAUUGGAUUGGCCAUCCGGUGACUAACAGAGCUAUUGUGUUCCAAUU | |
| | UGUUGGAUUUACCCCGCUCACACUCACAGUCGUAAGAACCCUUCAUUACGUGUUAUUUCUCAACUCA | |
| | AGAAA | |
| 194 | UCACCCUCUUUUCCGGUGGUCUGGUCCCGGACCACCGUUACUCCAUUCAGCUUCUUCGGAACCUGUU | Kobuvirus- |
| | CGGAGGAAUUAAACGGGCACCCAUACACCCCCAUCCCCUUUCUGCAACUUAAGUAUGUGUGCUCGUG | 16317 |
| | AUCUUGACUCCCACGGAAUGGAUCGAUCCGCUGGAGAACAAACUGCUAGAUCCACAUCCUCCCUUCC | |
| | CCUGGGAGGACCUUGGUCCUCCCACAUCCUCCCCCCAGCCUGACGUACCACAGGCUGUGUGAAGCCC | |
| | CCGCGAAAGCUGCUCACGUGGCAAUUGUGGGUCCCCCCUUCAUCAAGACACCAGGUCUUUCCUCCUU | |
| | AAGGCUAGCCCCGAUGUGUGAAUUCACAUUGGGCAACUAGUGGUGUCACUGUGCGCUCCCAAUCUCG | |
| | GCCGCGGAGUGCUGUUCCCCAAGCCAAACCCCUGGCCCUUCACUAUGUGCCUGGCAAGCAUACCUGA | |
| | GAAGGUGUUCCGCUGUGGCUGCCAGCCUGGUAACAGGUGCCCCAGUGUGCGUAACCUUCUUCCGUCU | |
| | UCGGACGGUAGUGAUUGGUUAAGAUUUGGUGUAAGGUCCAUGUGCCAACGCCCUGUGCGGGAUGAAA | |
| | CCUCUACUGCCCUAGGAAUGCCAGGCAGGUACCCCACCCCCGGGUGGGAUCUGAGCCUGGGCUAAUU | |
| | GUCUACGGGUAGUUUCAUUUCCAAUUCUUUUAUGUCGGAGUC | |
| 195 | UCACCCUCUUUCCCGGUGGUCUGGUCCCGGACCACCGUUACUCCAUUCAGCUUCUUCGGAACCUGUU | Aichivirus- |
| | CGGAGGAAUUAAACGGGCACCCAUACAUCCCCAUCCCCUUUCUGUAACUUAAGUAUGUGUGCUUGUA | Chshc7 |
| | AUCUUGACUCCCACGGAAUGGAUCGAUCCGCUGGAGAACAAACUGCUAGAUCCACAUCCUCCCUUCC | |
| | CCUGGGAGGACCUUGGUCCUCCCACAUCCUCCCCCCAGCCUGACGUACCACAGGCUGUGUGAAGCCC | |
| | CCGCGAAAGCUGCUCACGUGGCAAUUGUGGGUCCCCCCUUCAUCAAGACACCAGGUCUUUCCUCCUU | |
| | AAGGCUAGCCCCGAUGUGUGAAUUCACAUUGGGCAACUAGUGGUGUCACUGUGCGCUCCCAAUCUCG | |
| | GCCGCGGAGUGCUGUUCCCCAAGCCAAACCCCUGGCCCUUCACUAUGUGCCUGGCAAGCAUAUCUGA | |
| | GAAGGUGUUCCGCUGUGGCUGCCAGCCUGGUAACAGGUGCCCCAGUGUGCGUAACCUUCUUCCGUCU | |
| | CCGGACGGUAGUGAUUGGUUAAGAUUUGGUGUAAGGUUCAUGUGCCAACGCCCUGUGCGGGAUGAAA | |
| | UCUCUACUGCCCUAGGAAUGCCAGGCAGGUACCCCACCCUCGGGUGGGAUCUGAGCCUGGGCUAAUU | |
| | GUCUACGGGUAGUUUCAUUUCCAAUCCUUUUAUGUCGGAGUC | |
| 196 | GGCUUCGGCCCCCUCACCCUCUUUUCCGGUGGUCUGGUCCCGGACCACCGUUACUCCAUUCAGCUUC | Aichivirus- |
| | UUCGGAACCUGUUCGGAGGAAUUAAACGGGCACCCAUACACCCCCAUCCCCUUUUUUGCAACUUAAG | Goiania |
| | UAUGUGUGCUCGUAAUCUUGACUCCCACGGAAUGGAUCGAUCCGCUGGAGAACAAACUGCUAGAUCC | |
| | ACAUCCUCCCUCCCCCCUGGGAGGACCUCGGUCCUCCCACAUCCUCCCCCCAGCCUGACGUAUCACA | |
| | GGCUGUGUGAAGCCCCCGCGAAAGCUGCUCACGUGGCAAUUGUGGGUCCCCCCUUCAUCAAGACACC | |
| | AGGUCUUUCCUCCUUAAGGCUAGUCCCGAUGUGUGAAUUCACAUCGGGCAACUAGUGGUGUCACUGU | |
| | GCGCUCCCAAUCUCGGCCGCGGAGUGCUGUUCCCCAAGCCAAACCCCUGGCCCUUCACUAUGUGCCU | |
| | GGCAAGCAUAUCUGAGAAGGCGUUCCGCUGUGGCUGCCAGCCUGGUAACAGGUGCCCCAGUGUGCGU | |
| | AACCUUCUUCCGUCCCCGGACGGUAGUGAUUGGUUAAGACUUGGCGUAAGGUUCAUGUGCCAACGCC | |
| | CUGUGCGGGAUGAAACCUCUACUGCCCUAGGAAUGCCAGGCAGGUACCCCACCUUCGGGUGGGAUCU | |
| | GAGCCUGGGCUAAUUGUCUACGGGUAGUUUCAUUUCUAAUUCUUUCAUGUCGGAGUC | |
| 197 | ACCCUCUUUUCCGGUGGUCUGGUCCCGGACCACCGUUACUCCAUUCAGCUUCUUCGGAACCUGUUCG | Aichivirus- |
| | GAGGAAUUAAACGGGCACCCAUACACCCCCACCCCCUUUUUGCAACUUAAGUAUGUGUGCUCGUGAU | ETHP4 |
| | CUUGACUCCCACGGAAUGGAUCGAUCCGCUGGAGAACAAACUGCUAGAUCCACAUCCUCCCUUCCCU | |
| | UGGGAGGACCUCGGUCCUCCCACAUCCUCCCCCCAGCCUGACGUACCACAGGCUGUGUGAAGCCCCC | |
| | GCGAAAGCCGCUCACGUGGCAAUUGUGGGUCCCCCCUUCAUUAAGACACCAGGUCUUUCCUCCUUAA | |
| | GGCUAGUCCCGAUGUGUGAAUUCACAUUGGGCAACUAGUGGUGUCACUGUGCGCUCCCAAUCUCGGC | |
| | CGCGGAGUGCUGUUCCCCAAGCCAAACCCCUGGCCCUUCACUAUGUGCCUGGCAAGCAUAUCUGAGA | |
| | AGGUGUUCCGCUGUGGCUGCCAGCCUGGUAACAGGUGCCCCAGUGUGCGUAACCUUCUUCCGUCUUC | |
| | GGACGGUAGUGAUUGGUUAAGAUUUGGCGUAAGGUUCAUGUGCCAACGCCCUGUGCGGGAUGAAACC | |
| | UCUACUACCCUAGGAAUGCCAGGCAGGUACCCCACCCUCGGGUGGGAUCUGAGCCUGGGCUAAUUGU | |
| | CUACGGGUAGUUUCAUUUCCAAUUCUUCUAUGUCGGAGUC | |
| 198 | GUUACUCCAUUCAGCUUCUUCGGAACCUGUUCGGAGGAAUUAAACGGGCACCCAUACACCCCCACCC | Aichivirus- |
| | CCUUUUCUGCAACUUAAGUAUGUGUGCUCGUAAUCUUGACUCCCACGGAAUGGAUCGAUCCGCUGGA | DVI2169 |
| | GAACAAACUGCUAGAUCCACAUCCUCCCUUCCCCUGGGAGGACCCCGGUCCUCCCACAUCCUCCCCC | |
| | CAGCCUGACGUAUCACAGGCUGUGUGAAGUCCCCGCGAAAGCUGCUCACGUGGCAAUUGUGGGUCCC | |
| | CCCUUCAUCAAGACACCAGGUCUUUCCUCCUUAAGGCUAGCCCCGAUGUGUGAAUUCACAUUGGGCA | |
| | ACUAGUGGUGUCACUGUGCGCUCCCAAUCUCGGCCGCGGAGUGCUGUUCCCCAAGCCAAACCCCUGG | |
| | CCCUUCACUAUGUGCCUGGCAAGCAUAUCUGAGAAGGUGUUCCGCUGUGGCUGCCAGCCUGGUAACA | |
| | GGUGCCCCAGUGUGCGUAACCUUCUUCCGUCUCCGGACGGUAGUGAUUGGUUAAGAUUUGGUGUAAG | |
| | GUUCAUGUGCCAACGCCCUGUGCGGGAUGAAACCUCUACUGCCCUAGGAAUGCCAGGCAGGUACCCC | |
| | ACCUUCGGGUGGGAUCUGAGCCUGGGCUAAUUGUCUACGGGUAGUUUCAUUUCCAAUUCUUUUAUGU | |
| | CGGAGUC | |
| 199 | GGGUGGGGGGGUGUCGGCCCCCUCACCCUCUUUUCCGGUGGUCUGGACCCGGACCACCGUUACUCCA | Aichivirus- |
| | UUCAGCUUCUUCGGAACCUGUUCGGAGGAAUUAAACGGGCACCCACUUUCCUGUCCUCUCCCCUUUU | rat08 |
| | CUGUAACUCCAAGUGUGUGCUCGUAAUCUUGACUCCCGCGGAUUGACCGCUCCGCUGGUGAACAAAC | |
| | UGCUAGGUCAUCUCCUCCCCACCCUUGGGCGUCCUUCCGGGCGUCCACACCCUCCCCCCAGCCUGAC | |
| | GUGUCACAGGCUGUACAAAGACCCCGCGAAAGCUGCUAACGUGGCAAUUGUGGGUCCCCCCUUUGUA | |
| | AAGGAACCGAGUCUUUCUCCCUUAAGGCUAGACCCCUGUGUGAAUUCACAGGUGGCAACUAGUGGUU | |
| | CCACUGCAUGCUCCCGACCUCGGCCGCGGAGUGCUGUUCCCCAAGUCGUAACACUGACCUUCACUUA | |
| | UGUGCCUGGCAAGCAUAUCUGAGAAGAUGUUCCGCUGUGGCUGCCAAACCUGGUAACAGGUGCCCCA | |
| | GUGUGCGUAGUCUUCUUCCGUCUUCGGACGGUAGGUGUUAGGUAAAGAUGCGGCGUAAGGUUCAAGU | |
| | GCCAACGCCCUGGAAGGGAUGACCCUUCUACUGCCCUAGGAAUGCCGCGCAGGUACCCCAGGUUCGC | |
| | CUGGGAUCUGAGCGCGGGCUAAUUGUCUACGGGUAGUUUCAUUUCCCUCUUCUUCCACUGGCAUC | |
| 200 | GGGUGGGGGGGCCUCGGCCCCCUCACCCUCUUUUCCGGUGGUCUGGACCCGGACCACCGUUACUCCA | Aichivirus- |
| | UUCAGCUUCUUCGGAACCUGUUCGGAGGAAUUAAACGGGCACCCACUUUCCUGUCCUCUCCCCCUUU | Rt386 |
| | CUGCAACUCAAGUGUGUGCUCGUAAUCCUGACUCCCACGGGUUGACCGCCCCGUUGGUGAACAAACA | |
| | GCUAGGUCAUUCCCUCCCUACCCCUGGGCGCCAUUUCAGUGGCGUUCAUAUCCUCCCCCCAGCCUGA | |
| | CGUGUCACAGGCUGUGCAAAGUCCCCGCGAAAGCUGCUCACGUGGCAAUUGUGGGUCCCCCCUUUGU | |
| | GAAGGAACCGAGUCUUUCUCCCUUAAGGCUAGACCCCUGUGUGAACUCACAGGUGGCAACUAGUGGU | |
| | UCCACUGCAUGCUCCCGACCUCGGCCGCGGAGUGCUGUUCCCCAAGUCGUGACACUGACCUCCACUU | |
| | AUGUGCCUGGCAAGCAUAUCUGAGAAGAUGUUCCGCUGUGGCUGCCAAACCUGGUAACAGGUGCCCC | |
| | AGUGCGUGUAGUCUUCUUCCGUCUCCGGACGGUAAGUGUGUGGUAAAGAUGCGGCGUAAGGUUCAAG | |
| | UGCCAACGCCCUGGAAGGGAUGACCCUUCUACUGCCCUAGGAAUGCCGCGCAGGUACCCCAGGUUCG | |
| | CCUGGGAUCUGAGCGCGGGCUAAUUGUCUACGGGUAGUUUCAUUUCCCUCUCUUUUCACUGGCAUC | |
| 201 | UUUGAAAAGGGGGUGGGGGGGCCUCGGCCCCCUCACCCUCUUUUCCGGUGGCCAUUCGCCCGGGCCA | Porcine- |
| | CCGUUACUCCACUCCACUCCUUCGGGACUGGUUUGGAGGAACACAACAGGGCUUCCCAUCCCUGUUU | Kobuvirus- |
| | ACCCUUUAUUCCAUCAUCCUUUCCCCAAGUUUACCCUAUCCACACCCCACUGACUGACUCCUUUGGA | GS2 |
| | UUUUGACCUCAGAAUGCCUAUUUGACCUCCCACUCGCCUCUCCCUUUUCGGAUUGCCGGUGGUGCCU | |
| | GGCGGAAAAAGCACAAGUGUGUUGCAGGCUACCAAACUCCUACCCGACAAAGGUGCGUGUCCGCGUG | |
| | CUGAGUAAUGGGAUAGGAGAUGCCUACAACAGGCUCGCCCAUGAGUAGAGCAUGGACUGCGGUGCAU | |
| | GUGACUUCGGUCACCACGGGCAUAGCAUUGCUCACCCGUGAAUCAAGUCAUCGAGAUUUCUCUGACC | |
| | UCUGAAGUGCACUGUGGUUGCGUGGCUGGGAAUCCACGCUUGACCAUGUACUGCUUGAUAGAGUCGC | |
| | GGCUGGCCGACUCAUGGGUUAAAGUCAGUUGACAAGACAC | |
| 202 | UUUCACACCCUCUUUUCCGGUGGUCCGGACCCAGACCACCGUUACUCCAUUCAGCUACUUCGGUACC | Kobuvirus- |
| | UGUUCGGAGGAAUUAAACGGGCACCCUACCCAAGGGUUACAUGGGACCAUAUUCCUCCUCCCCUGUA | SZAL6- |
| | ACUUUAAGUUUUGUGCCCGUAUUCUUGACUCCAGGCGGAUGUUGUGUCGCCCGUCCUGUGAACAAAC | aichivirusA5 |
| | AGCUAGACACUUUCCUCCCCUCCCUCUGGGCUGCUCCGGCAGUCCACUCCCUCCCCCCAGCGUAACA | |
| | UGCCCCGCUGGAGUGAUGCACCUGGAAGUCGUGGACGUGGGUUAGUAACUUCGGUGAAAACCCACUA | |
| | UAAUGACAACUGGUUGACCCCCACACUCAAAGGACUCGAGUCUUUCUCCCUUAAGGCUAGCCCGGCC | |
| | ACAUGAAUUUGCAGCUGGCAACUAGUGAGUCCACCAUGUCCCGCAACCUCGGCUGCGGAGUGCUGUU | |
| | CCCCAAGCGUAUGCCUUCCUUCUGUAAGAGUGCGCCUGGCAAGCACAUCUGAGAAGUCGUUCCGCUG | |
| | CGUCGUGCCAACCUGGCGACAGGUGACCCAGUGUGCGUAGACUUCUUCCGGAUUCGUCCGGCUCUUC | |
| | UCUAGGAAACAUGCGUGUAAGGUUCAUGUGCCAAAGCCCUGCGCGCGGUGUUCUUCUACUGCCCUAG | |
| | GAAUGUGCCGCAGGUACCCCUACUUCGGUAGGGAUCUGAGCGGUAGCUAAUUGUCUACGGGUAGUUU | |
| | CAUUUCCAUCUUCUCUUCAGGUCGACAUC | |
| 203 | UUUGAAAAGGGGGUGGGGGGGCUUCGGCCCCCUCACCCUCUUUUCCGGCGGCGCAAGUUCGCGUCGU | Kobuvirus- |
| | CGUAAGCCUGGACACCCAUGUUCGCAUCGGUCUCCGGGCAAAUACAAGAACCCCAGACCUUCUGGUA | sheep-TB3 |
| | CUUCUUCGCCUGGGUCACAAAAGCGAAGAACCUGCCUCUCUAACGCCAGACGAGCGGCAUUAAACUU | |
| | GAACUUCUGGCACUCUCCACUCUCCCUUUUCCCUGUCCCUUUCCCCACUGCGCUCUCAAGGUCGCGC | |
| | AAUCCUGGGACUAGCCCAGUUUUAAAAGUUCCUGGCACCCUUUGCCCCUCUAGGCCCUUAAGGUAGG | |
| | AACUGACCUUGUGCUGUGAUCUCGGUGCGGGAGUGCUACCACGUAGUCAUCGUAAGCCUCGUUUCUG | |
| | GUUCUGCCCUGGCAAGGCUACAGAGUACCGUGUUCCGCUGUGGAUGCCAUCCGGGUAACCGGACCCC | |
| | CAGUGUGUGUAGCGGUAUGUUCACGGUCCGCCGUGUUCACCAGAUUCCUGACCUGGCUUUGCUAGAA | |
| | AUGGUGUGUGCCCAAUCCCUGUGACCAGUAUCAAUUACAUCACCUAGGAAUGCUAGGAAGGUACCCC | |
| | AGUCCUGAGCUGGGAUCUGAUCCUAGGCUAAUUGUCUACGGUGAUGCUCCUUUUAUUUUCUUACAAC | |
| | UGCUAUUGACUGUCUGAUUGCUGAUUCUGCUCUUGUGCUCUUCUGCUCUGGCUCAUUCUCAAGGGUU | |
| | CUCUUUGUCCAAGAUCCUUUGGUUCUCUCCUUGUUCCACUUGCCACUGCCAACGCUUGUC | |
| 204 | GACAGGUGUUUUGGAGGGCGGAUGACGAUAUCUGGCUGGCCACCAGGGAAUAACGGCAAAUGUCUGA | Bat- |
| | UCAUACGGUUCACAAGUCUACCGGCGAUAGUGGUUCAACACCAUGUGUAGCAGGGAUUCUUGCGUAU | sapelovirus |
| | GUGAAGGCGACAGUGC | |
| 205 | GAAAGGGGGUGGUAGGGGCCGUACGGUCAUGCCGUGCGGUUCCGCCACCCCUAGGGGGCCACACGGU | Bovine- |
| | CCUGCCGUGUGGUUCCCGCUGGUUGUACAGUGACGCAUUGGGGGCCGUACGGUCCUGCCGUGCGGUU | cardiovirus- |
| | UCCUUUGCUUGCUGUGCAAUCGGGGAUGACACCCCCUUUCAACGUGGGUACUACGAAAGUGCCCCUC | 1 |
| | GCUCCGAGGUUAAAGGAGAACCCCCCCUUCUUACCCCCACUCAGCUCGCCCUUCAGUGCGGGCGCUA | |
| | GCCUUUCCACUUGCAGCUUCUGCUUGUAGAUGCUUGCACCGUGAUUGGUGCGCUUCUUGCUUUAGUC | |
| | GCUUGUGCUUCUAUCGUUCUGACGAUUCAGUUUCCUAACGCCAGUGUUUCGACGGCCCAAGGGGGUA | |
| | GUUGCGGUUAGUAUUCCUACCGCAAUUAUCCCUUUCCCCGUUCGUAGCUGGUUUGGAUCUUGGAUCU | |
| | CUCUCCUUCCUUCCCCCGUCUUCAAUUUAGCUUCGUGAUUGAAGCAUCUCACUGUCUCUAGUAUUUA | |
| | UGUCGGACUGACGAUUGAGUACGUUCAGAUUGUGUUUGGGAGGCCCAAGGGAUCGAUGGACAACACU | |
| | UCGAAAGAGUCACUUGUCCACCGCUCCUUUCCCCUACCCUAGCAACUCUGGAUUUGCUCACGUGGAG | |
| | UUCGAGGUCUGUACUUUAACUCUGACUUGCUUUUCUUACCUUGCUAUCUUGCUGACGUGGAUUGGUU | |
| | GUAGACUGAUUCACGUUCUCGUUAGAUGCUGACGUGGAGUACGAUCGCUGUACAUUCCACUACUGCC | |
| | AAUUAGCUCCCCCUUCCCGUUGCUCCCCUCUAUAAGGAGAGCCUUCUCUUGCAAAGGUGAAGCCUUC | |
| | ACCCCCGGUCGAAGCCGCUUGGAAUAAGACAGGGUUAUUUUCUCCUCUCCUCGGCGCUUGCCUCUUC | |
| | UAAGCUGAAUAGGUUCUAUCUAUUCAGGCGGAUGGUCUGGUCCGUUCCUUCUUGGACAGAGUGUGUA | |
| | UCUGGGUUUUCCGGAUCUCGACCACACACUCACCAGAGCUCAGGAGUGAUUAAGUCAAGGCCCGAUC | |
| | UGCGGCGAAAAGGAAAUGAAGUAUUUUGCAGCUGUAGCGACCUCUCAAGGCCAGCGGAUUUCCCCAC | |
| | CUGGUGACAGGUGCCUCUGGGGCCAAAAGCCACGUGUUAAUAGCACCCUUGAGAGCGGUGGUACCCC | |
| | ACCACCCUGCAAAUUAUGGAUUUGACUUAGUAACUAAAAGAUUGACUUGGCAUACCUCAACCUGAGC | |
| | GGCGGCUAAGGAUGCCCUGAAGGUACCCGUGUUGAAAUCGCUUCGGCGACCAUGGAUCUGAUCAGGG | |
| | GCCCUGCCUGGAGUGGUUCUAUCCCACACAGCGUAGGGUUAAAAAACGUCUAACCGCCCCACAAAGA | |
| | CCCCGGCAGGGAUGCCGGUUUCCUUUUUACCAAUUCUUGACACU | |
| 206 | UUGGAUCUGAGCAGGGGCCCCCUUUGGGUUGCUUUACAACUCAACUGGGGGUUAAAAAACGUCUAAC | Bovine- |
| | CCGACACGCCAGAGGGAUCUGGUUUCCUUUUAUUUCUUUUACUCACCACUGGAUGCAGAUUGACGAU | picornavirus- |
| | AAACGUUGUUGUUUGUGACUAUUGACUUGAUCUGCUUCUACGGGUUUACUUUCACUGUUAUACUUCU | isolate- |
| | UGCUUUGUUUGGUGUUCACUGUACUUUGUCUCCUUCUACAUUUCACA | TCH6 |
| 207 | UUUAAGUGUUGUGCCCAAUCUCUUGACUCCUGCUGGAACCACCGACCAGUAGUGUCCAAAAUGCCAG | Canine- |
| | GUGGAAAAUCCUCCCUUCCCCUCUGGGCUUCAUGCCCGGCAUCCUCCCCCCAGCCUGACGUGCCACA | kobuvirus |
| | GGCUGUGCAAAGACCCCGCGAAAGCUGCCAAAAGUGGCAAUUGUGGGUCCCCCCUUUGUCAAGGCGU | |
| | CGAGUCUUUCUCCCUUAAGGCUAGUCCUGUCAGUGAACUCUGUCGGGCAACUAGUGACGCCACUGCA | |
| | UGCCUCCGACCUCGGCCGCGGAGUGCUGCCCCCCAAGUCAUGCCCCUGACCACAAGUUGUGCUGUCU | |
| | GGCAAACAUUGUCUGUGAGAAUGUUCCGCUGUGGCUGCCAAGCCUGGUAACAGGCUGCCCCAGUGUG | |
| | CGUAAUUCUCAUCCAGACUUCGGUCUGGCAACUUGCUGUUAAGACAUGGCGUAAGGGGCGUGUGCCA | |
| | ACGCCCUGGAACGAGUGUCCACUCUAAUACCCCGAGGAAUGCUACGCAGGUACCCCUGGCUCGCCAG | |
| | GGAUCUGAGCGUAGGCUAAUUGUCUAAGGGUAUUUUCAUUUCCCACCCUCUUCUUCUUGUUCAUA | |
| 208 | UUAAAAUAGCCUGAGGGUUGUUCCCACCCACAGGGCCCACGUGGCGCAGUACUCUGGUAUCACGGUA | Enterovirus- |
| | CCUUUGCACGCCUAUUUUAUACCCCUUCCCCAUCGUAACUUAGAAGCAACAAACAAACUGCCCAAUA | sp-isolate- |
| | GCAGCACAACACCCAGUUGUGUUAGGGGCAAGCACUUCUGUUUCCCCGGAAGGGUCUGACGGUAUGC | CPML |
| | UGUACCCACGGCAGAAGUAUGACCUACCGUUAACCGGCCAUGUACUUCGAGAAGCCUAGUACCAUUA | |
| | UGAAGGUUGAUUGAUGUUACGCUCCCCAGCAACCCCAGCUGGUAGUUCUGGUCGAUGAGUCUCGGCA | |
| | UUCCCCACGGGCGACCGUGGCCGAGGCUGCGUUGGCGGCCAGCCUACACCAUACGGUGUAGGACGUC | |
| | AAGAUACUGACAUGGUGUGAAGAGCCUAUUGAGCUACGUGGUAGUCCUCCGGCCCCUGAAUGCGGCU | |
| | AAUCCUAACUCCGGAGCAUCCGCCAGUAAGCCCACUGGAAGGGUGUCGUAAUGCGAAAGUCUGGAGC | |
| | GGAACCGACUACUUUGGGUGUCCGUGUUUCCUGUUUUACUUAUUGUUUGGCUGCUUAUGGUGACAAC | |
| | UUAUAGUUAUCAUCAUAAGCUACUUGGUCUUGCCAACCGGAGAAUUAUUUGGUUAUUUGUUGGUUUC | |
| | AUAAACCUACAGUCGUAUUUUCCUGUCUUAUUAAUUGUUCUCAAAAUUAACAACA | |
| 209 | UUAAAACAGCCUGGGGGUUGUUCCCACCCCCGGGGCCCACGUGGCGCCAGUACUCUGGCACGUUAGU | Enterovirus- |
| | GCCCUUGUACGCCUGUUUUCCCCUCCUUAAACAAAUUAAGAUUGCCACUACUGAGGGGAGUAGUCCG | AN12-F7 |
| | ACUCCGCUCCAGUACCGCUGCACCAGUGAGCUGGUACGCUAGUACCUUCGCACGGAGUAGAUGGCAU | |
| | CCCCCACCCCGUAACUUAGAAGCAAAGUACACAUCUGGCCAAUAGUGGCGCUGCAUCCAGCCGCGCA | |
| | ACGGUCAAGCACUUCUGUUUCCCCGGUCCGCAAGGGUCGUUAUCCGCCCAGUCCACUACGGAAAGCC | |
| | UACUAACCAUUGAAGCUAUCGAGAGGUUGCGCUCGGCCACGACCCCGGUGGUAGCUCUGAGUGAUGG | |
| | GGCUCGCAAACACCCCCGUGGUAACACGGAUGCUUGCCCGCGCGUGCACUCGGGUUCAGCCUAUUGG | |
| | UUGUUCACCUCAACAUAGUGUAAAUGGCCAAGAGCCUACUGUGCUGGAUUGGUUUUCCUCCGGAGCC | |
| | GUGAAUGCUGCUAAUCCCAACCUCCGAGCGUGUGCGCACAAUCCAGUGUUGCUACGUCGUAACGCGU | |
| | AAGUUGGAGGCGGAACAGACUACUUUCGGUACUCCGUGUUUCUUUUGUUUUAUUUUGAAUUUUAUGG | |
| | UGACAAUUGCUGAGAUUUGCGAAUUAGCGACUCUACCGCUGAACAUUGCCCUGUACUACCUAAUCGC | |
| | AUUUCACAAAACCUCAGAGAUACCAAGCUCUUACAUUGAUCUGCUUGUUUUCCUGAAUCUCAAAUAU | |
| | AAAUUGGAACAAGCAAA | |
| 210 | UUAAAACUGGGUGUGGGUUGUUCCCACCCACACCACCCAAUGGGUGUUGUACUCUGUUAUUCCGGUA | Human- |
| | ACUUUGUACGCCAGUUUUUCCCUCCCCUCCCCAUCCUUUUACGUAACUUAGAAGUUUUAAAUACAAG | rhinovirus-1 |
| | ACCAAUAGUAGGCAACUCUCCAGGUUGUCUAAGGUCAAGCACUUCUGUUUCCCCGGUUGAUGUUGAU | |
| | AUGCUCCAACAGGGCAAAAACAACAGAUACCGUUAUCCGCAAAGUGCCUACACAGAGCUUAGUAGGA | |
| | UUCUGAAAGAUCUUUGGUUGGUCGUUCAGCUGCAUACCCAGCAGUAGACCUUGCAGAUGAGGCUGGA | |
| | CAUUCCCCACUGGUAACAGUGGUCCAGCCUGCGUGGCUGCCUGCGCACCUCUCAUGAGGUGUGAAGC | |
| | CAAAGAUCGGACAGGGUGUGAAGAGCCGCGUGUGCUCACUUUGAGUCCUCCGGCCCCUGAAUGCGGC | |
| | UAACCUUAAACCUGCAGCCAUGGCUCAUAAGCCAAUGAGUUUAUGGUCGUAACGAGUAAUUGCGGGA | |
| | UGGGACCGACUACUUUGGGUGUCCGUGUUUCACUUUUUCCUUUAUUAAUUGCUUAUGGUGACAAUAU | |
| | AUAUAUUGAUAUAUAUUGGCAUC | |
| 211 | ACCUGCGACAGUGCCAACCCACAUCUCUUCCCACUAUUAUUAUUCCGGUUCCCGCCUUAGGUCCAUU | Human-TMEV- |
| | GAAAAGACCAAACAUGUCAUCCCUUGGAUGCUGGCUAAUCAGAGGAAAGUCAGCAUUUUCCGGCCCA | like- |
| | GGCUAAGAGAAACAUAUAAGUUAGAAAUGAUUCUAAUCCCUUGACGAAUUCGGAACGAGAAGUUCUC | cardiovirus |
| | CCUCCCUUGCCGCUUGUUCACACCCAUCAUUUAAUUCGGCCCUUUGUGACAAGCCCCUCGGUGAAAG | |
| | GACCUCUCUCUUUCCGACGUGGUUGGAAUUAACAUCUUUUCCGACGAAAGUGCUAUUAUGCCUCCCC | |
| | GAUUGUGUGAUGCUUUCUGCCCUGCUGGGCGGAGCGUCCUCGGGUUGAGAAACCUUGAAUCUUUUCC | |
| | UUUGGAGCCUUGGCUCCCCCGGUCUAAGCCGCUUGGAAUAUGACAGGGUUAUUUUCCAAACUCUUUA | |
| | UUUCUACUUUCAUGGGUUCUAUCCAUGAAAAGGGUAUGUGUUGCCCCUUCCUUCUUUGGAGAAUCUG | |
| | CGCGGCGGUCUUUCCGUCUCUCAACAGGCGUGGAUGCAACAUGCCGGAAACGGUGAAGAAAACAGUU | |
| | UUCUGUGGAAAUUUAGAGUGGACAUCGAAACAGCUGUAGCGACCUCACAGUAGCAGCGGAUUCCCCU | |
| | CUUGGCGACAAGAGCCUCUGCGGCCAAAAGCCCCGUGGAUAAGAUCCACUGCUGUGAGCGGUGCAAC | |
| | CCCAGCACCCUGGUUCGAUGGCCAUUCUCUAUGGAACCAGAAAAUGGUUUUCUCAAGCCCUCCGGUA | |
| | GAGAAGCCAAGAAUGUCCUGAAGGUACCCCGCGCGCGGGAUCUGAUCAGGAGACCAAUUGGCAGUGC | |
| | UUUACGCUGCCACUUUGGUUUAAAAACUGUCACAGCUUCUCCAAACCAAGUGGUCUUGGUUUUCCAA | |
| | UUUUGUUGACUGACAAU | |
| 212 | UUGAAAAGGGUGUGGGGGGGGAUACCCUCCCCCUCCACACUCUUUUCGACGGCGCGCCUCGGCAAGG | Kagoshima-2- |
| | CCGCCGAUACCGCACUGUGCUACUUCGGUACCACUCUGCGGGAAUGACAAUAUGCCCGGUGUAUAGU | 24-KOV |
| | UUGCCUGUUUCUCGCACCGUUACCGCUCGUUCGGGAAUGUGAAACUGGCACCCCUCCUCUCCCCUAC | |
| | CACCCUUUCUCCUUCGCCCCCAUUCAUAAUUUACAACGCCGCACACAGCGGCGGCCGCCAAGGGCUA | |
| | GCCUGGCGGUUAUAAAAGGAACCUGGGUCUUUCCCUCUUCAAGCCAAAAGGUAGGUUCCCUGUGUCC | |
| | CUGAAUGCUCGGUGAGGAAUGCUGCACCGUAACGCUUUGUGAAGUGUUUGCAAGUUCUGGCCCGGCA | |
| | AGCCUACAGAGUGCUGUGAUCCGCUGCGGACGCCAUCCUGGUAACAGGACCCCCAGUGUGCGCAACA | |
| | GUAUGUUCAGACUUCGGUUUGUUCACUUGCUUUCAUGGACCAUUGCGCGAAAGUGCGUGCGCCAUAU | |
| | CCCUGUACUUCAGGUGUGCUUCUCUGGACCCUAGGAAUGCUGCGAAGGUACCCCGUUUCGGCGGGAU | |
| | CUGAUCGCAGGCUAAUUGUCUAUGGGUUCAGUUUCCUUUUUCUUUACUCCACAAUUGACUGCUUAAC | |
| | UGACUCUGGAUCUUGUGCUUCCACUGCUCUUUCUGCUCUCAAAACGGCUUCACUUACCAACUCUCAC | |
| | CUUUCGACCAACACCAUUUACACACUAACUUUUUUCGACUCUUCUGACUCCUGGCUUGGUGAAGAC | |
| 213 | CUCUCUUUGACCCUGGCCACUGGGGUUCAGUGGCACUACAUCUCUCUCUCUUCUUUAUCUAUAUCGU | Lesavirus-1 |
| | UUCUUUACUUUACUUUACUUUACUAUAUUUCUUACUUUACUAUAUUUCUUACUUUACUUAUCUAUAU | |
| | AUAUCUAAUCUUUACUAUCUUUUCUAACUUUAUUUUCUUAUUUCUUAUCUUUAUUCUUUUGUAACUA | |
| | AUAAGCAAGUUUUACUGCCUGCAAACUGCUUCAAUGGGACCACCGCUUCGGCGACCCCUUUGUUGAG | |
| | UUUGUAUGUUUUUAAGUAAUCUUUGCAACCAUACGAUUAUUUUAGCCGCCUCUCUAUAAUGAUCUUG | |
| | UUAUAGUGGGACGUGAAACAUUGGUUUUUCUCACACACGUCCGGUCACCCGGGCGUGUGUUCUUCCG | |
| | UAAGUCCUAUCCACAUACCAUCGUGGGUAGGCCAGCAUGUUUGCACAGGCUGUGACAGUGUGGGUGG | |
| | GCUUUCCACCUCUCAACAACACACUGAAUUGCAAUGCACUCACGGAGGAAAUGACAAUUUGGUUAUA | |
| | GUUUUGAACUGUGCUAGUAAUUUCUUUCACAUUAAGCCAUGUUGCCUGCGGAAUCACAUGUGGUAAC | |
| | ACAUGCCUCAGGGCCCAAAAGGCACGGGUUAGCAGCCCCUUCAUGGUGUGUUAGAAGUGAAAACACA | |
| | UAGUAUGAGCUAUAAUAUCUUUGUUGUCUUCUCUGUAGUGUACCCCGCCAAAUGUAAGGAUGCCCAG | |
| | CAGGUACCCAUUUUAUGGAUCUGAGCUGGGGAUUGAUAGUGUAUCUAUAAAUGCACUGAUCAAUUUA | |
| | AAAAGCGUCUAAGUUUGGCACAAACACUGGGGACAGUGUUUUUCCUUUAUUCUUUUAUUUGAUUA | |
| 214 | ACACUCAUUUCCCCCCUCCACCCUUAAGGUGGUUGUAUCCCCUACACCCUACCCUCCCUUCCACAUA | Porcine- |
| | GGACGAAUAAACGGACUUGAGAUUAAGGCAAGUACAUAAGGUAUGGUUUUUGGAUACACUUAAAUGG | sapelovirus |
| | CAGUAGCGUGGCGAGCUAUGGAAAAAUCGCAAUUGUCGAUAGCCAUGUUAGCGACGCGCUUCGGCGU | |
| | GCUCCUUUGGUGAUUCGGCGACUGGUUACAGGAGAGUAGACAGUGAGCUAUGGGCAAACCCCUACAG | |
| | UAUUACUUAGGGGAAUGUGCAAUUGAGACUUGACGAGCGUCUCUUUGAGAUGUGGCGCAUGCUCUUG | |
| | GCAUUACCAUAGUGAGCUUCCAGGUUGGGAAACCUGGACUGGGUCUAUACUGCCUGAUAGGGUCGCG | |
| | GCUGGCCGCCUGUAACUAGUAUAGUCAGUUGAAAACCCCCC | |
| 215 | UUAAAACAGCCUGUGGGUUGUUCCCACCCACAGGGCCCACUGGGCGCUAGUACACUGGUAUGCCUUA | Porcine- |
| | CCUCGGUAUUACGGUACCGAUGUAUAUAGUACCUUUGUACGCCUGUUUUAUAUCCCUCCCCCACAUG | enterovirus- |
| | UAACCUUAGAAGUUUACACAAACAAAGACCAAUAGGAGUCCAACACCCAGUUGGAUUGCGGUCAAGC | 9 |
| | ACUUCUGUUUCCCCGGACCUAGUAGUGAUAGGCUGUACCCACGGCCGAAGAUGAACCCGUCCGUUAU | |
| | CCGGCUACCUACUUCGGGAAGCCUAGUAACAUUCUGAAGUCUCUGAGGCGUUUCGCUCAGCACGACC | |
| | CCGGUGUAGAUCGGGCUGAUGGGUCUCCGCAUACCCCACGGGCGACCGUGGCGGAGGCCGCGUUGGC | |
| | GGCCCGCCUAUGGCGAAAGCCAUAGGACGCCUCUUAGAUGACAGGGUGUGAAGAGCCUACUGAGCUG | |
| | GGUAGUAGUCCUCCGGCCCCUGAAUGCGGCUAAUCCUAACCACGGAGCGUCCACCAGCAAUCCAGCU | |
| | GGCAGGGCGUCGUAACGGGCAACUCUGUGGCGGAACCGACUACUUUGGGUGUCCGUGUUUCCUUUUG | |
| | AUCCUAUUUUGGCUGCUUAUGGUGACAACGAUAAGUUGUUAUCAUAAAGCUCUUGGGUUGGCCACCU | |
| | GGAAAAAGUUAUCAGUGUUUGAUAUUGUUCGGCUCUCACGCCUACCAAUAAGACAAGCCCUAUAUUU | |
| | ACUUGUUGCAUUUUACUCGUCAGAAGAAAUCACAGAGUAUCAUUUGGAUUUGUUACUCACAUUAAGG | |
| | ACAAG | |
| 216 | UUGAAAGAAACUCCGGGUAUUAACCAUACCCGGAGUUUUGGCAGGGUGUUCCAACCUGUCCUGGCCA | Rabovirus-A |
| | UGCAUUUAUCUACGGAUAUUUGCAUGACCCGCUUUCUAUCGCCCCAACAAUCCCCUUUGUAACCACA | |
| | AGCUUUACUCAGGCUAGCAGCCCGACUAGCUGUUUGGAAGAAAAGGCUAGGGCACACACCAACAACA | |
| | CCGACCCCACUGGUCGAAGGCCGCUUGGCAAUAAGACUGGUGGAACAGGGUCGCCUGUAGUUGUUUG | |
| | GAACAUUCUUUCUAAUGACUUUGUCAGCGGUGCUACUCACACCGUAACUCUUCUACCCUAUCCCCAC | |
| | GCUUGUGGAACUAGGAGGGGAUGAGUGAUUCAAGUAAGUACUGUCAGAAUGGUGAAAAUGAUCUGAU | |
| | UCUGAAACGCUAUGGAUCCAUCGAAAGAUGGGGCUACACGCCUGCGGAACAACACAUGGUAACAUGU | |
| | GCCCCAGGGGCCGAAAGCCACGGUGAUAGGAUCACCCGUGUAGUUUGAGAUCAUAUCAAUGUUCAUA | |
| | GUCUAGUAAGAUGAUUUGAAAUCUAACUGAGCUGAUGGCUAACUGCUUGUCUUAUUGCGGCCUAAGG | |
| | AUGUCCUGCAGGUACCUUUAGAUAACCUUAAGAGACUAUUGAUCUGAGCAGGAGCCAAAGUGGUCUU | |
| | UCCCAGCUUUGGUUAAAAAACGUCUAAGCCGCGGCAGGGGGGGGAGGCCCCCUUUCCUCCCAAAAC | |
| | UUAAUAUUGAUUGU | |
| 217 | UUUAAACAGUCUGGGGGUUGUUCCCACCCCUGGGGCCCACGUGGCGCUAGCACUCUGGUACGCUAGU | Yak- |
| | ACCUUUGUACGCCUGUUUUCCCCACCCUUAAAUAAAUUAAGAUUACCACUACCGAGGGGAGUAGUCC | enterovirus |
| | GACUCCGCUCCGGCACAGCCGCACCAGUGCACUGGUACGCUAGUACCUUUUCACGGGGUAGUCGGUA | |
| | UCCCCCCCCGUAACUUAGAAGCAUGUAACAAACCGACCAAUAGGUGCGCGGCAGCCAGCUGCGUUGC | |
| | GGUCAAGCACUUCUGUCUCCCCGGUCCGCAAGGAUCGUUACCCGCCCACUCCACUACGAGGAGCCUA | |
| | GUAACUGGCCAAGUGAUUGCGGAGUUGCGUUCAGCCACAACCCCAGUGGUAGCUCUGGAAGAUGGGG | |
| | CUCGCACAUCCCCCGUGGUAACACGGUUGCUUGCCCGCGUGUGCUUCCGGGUUCAGUCUCCGACUGU | |
| | UCACUUCAACAUCACGCAACCAGCCAAGAGCCGAUUGUGCUGGAGUGGUCUUCCUCCGGGGCCGUGA | |
| | AUGCUGCUAAUCCUAACCUCCGAGCGUGUGCGCACAAUCCAGUGUUGCUACGUCGUAACGCGUAAGU | |
| | UGGAGGCGGAACAGACUACUUUCGGCACCCCGUGUUUCCUUUAUUUUAUUCUUAUUUUAUGGUGACA | |
| | AUUGCAGAGAUUUGUGAUAUUGCGACUUUACCGUUAAACAUAGCACUGCAUUACCUGGUUGCAUUCC | |
| | ACAAAACUUCAGAGAUUCCUAGUUCCUACAUUGACCUACUUGUUUAUUUGAAUCUUAAAUACAAACU | |
| | UGAGCAAGUGAA | |
| 218 | CCCCUAGGAUCCACUGGAUGUCAGUACACUGGUAUCGUGGUACCUUUGUACGCCUGUUUUAUACCCC | Human- |
| | CUUCCCCGCAACUUUAGAAGCAUCAAAAGCACCGCUCAAUAGUCACCACACCCCCAGUGUGGUUUCG | enterovirus- |
| | AGCAAGCACUUCUGUUUUCCCGGUUGCGUCCCAUAUGCUGUGCAAACGGCAAAAAGGGACAAUAUCG | C105 |
| | UUACCCGCUUGUAUACUACGGGAAACCUAGUACCACCAUUGAUUGUGUUGAGAGUUGCGCUCAUCAC | |
| | CUUUCCCCGGUGUAGCUCAGGCCGAUGAGGCUCAGAAUCCCCCACAGGUGACUGUGUCUGAGCCUGC | |
| | GUUGGCGGCCUGCCCUCGCCUUAUGGCGUGGGACGCUUGAUACAUGACAUGGUGCGAAGAGUCUACU | |
| | GUGCUAUGCAAGAGUCCUCCGGCCCCUGAAUGUGGCUAAUCCUAACCACUGAUCCCACGCACGCAAA | |
| | CCAGUGUGUAGUGGGUCGUAACGCGCAAGUCGGUGGCGGAACCAACUACUUUGGGUGACCGUGUUUC | |
| | CUUUAUUACUUAUUGAAUGUUUAUGGUGACAAUUGUUUGAUUCAGUUGUUGCCAUUCUCUACAUUCA | |
| | UUUACCCAGCAUCAAACCAAUUGAACUGUUACA | |
| 219 | AGUCUGGACAUCCCUCACCGGCGACGGUGGUCCAGGCUGCGCUGGCGGCCUACCUGUGGUCCAAAGC | Human- |
| | CACGGGACGCUACAUGUGAACAAGGUGUGAAGAGCCUAUUGAGCUACAAAAGAGUCCUCCGGCCCCU | poliovirus- |
| | GAAUGCGGCUAAUCCCAACCACGGAUCAAGGGUGCACAAACCAGUGUACACCUUGUCGUAACGCGCA | 1-strain- |
| | AGUCUGUGGCGGAACCGACUACUUUGGGUGUCCGUGUUUCCUUUUUAAUUUUGAUGGCUGCUUAUGG | NIE1116623 |
| | UGACAAUCAUAGAUUGUUAUCAUAAAGCUAAUUGGAUUGGCCAUCCGGUGAGAGUGAAAUAUAUUGU | |
| | UUACCUCCCUGUUGGGUUUACUCUAACUAACUUCUCCAUUUAUAAACUUGUCAUCACAGUUUUAAUA | |
| | AUUAGAAGUGCAGUUUACA | |
| 220 | UUUAAAACAGCCUGGGGGUUGUUCCCACCCCCAGGGCCCACUGGGCGUUAGUACUCUGGUAUCGCGG | Human- |
| | UACCUUAGUAUGCCUGUUUUAUGUCUCCUUUCCCCCGCAACUUUAGAAGUAAUCAAGUUAUGGCUCA | enterovirus- |
| | ACAGUCGCCACACCCCCAGUGUGGUUCCGAGCAAGCACUUCUGUUCCCCGGUUGCGUCUUAUAUGCU | 109 |
| | GUGUGAACGGCAGAAAGGGACAAUAUCGUUAUCCGCUCAACUACUACGGGAAGCCUAGUACCACCAU | |
| | GGAUUGACCUGAAAGUUGCGUUCAGCGCACCCCCAGCGCAGCUCAGGCCGAUGAGGCUCCGAAUACC | |
| | CCACGGGCGACCGUGUCGGAGCCUGCGUUGGCGGCCUGCCCACGUUGCAAAACGUGGGACGCUCAUU | |
| | UCAUGACAUGGUGCGAAGAGCCUACUGUGCUAGUUGAGAGUCCUCCGGCCCCUGAAUGUGGAUAAUC | |
| | CUAACCACUGAACCUACGGGCGCAAACCAGCGUCUGGUAGGCCGUAACGCGCAAGUCGGUGGCGGAA | |
| | CCAACUACUUUGGGUGUCCGUGUUUCCUUUUAUCUUUUUGAAUGUUUAUGGUGACAAUUGUUGUGUA | |
| | CAGUUGUUACCAUAGUUUGCAUUCAGAAAUAAACCUAACACUUUCCAAUUAUUUGUUACA | |
| 221 | UUAAAACAGCUCUGGGGUUGUACCCACCCCAGAGGCCCACGUGGCGGCUAGCACUCCGGUAUCACGG | Human- |
| | UACCCUUGUGCGCCUGUUUUAUAUUCCCCCCCCGCAACUUAGAAGCACGAAACCAAGUUCAAUAGAA | poliovirus- |
| | GGGGGUACAAACCAGUACCACCACGAACAAGCACUUCUGUUUCCCCGGUGACAUUGCAUAGACUGCU | 2-strain- |
| | CACGCGGUUGAAAGUGAUCGAUCCGUUACCCGCUUGUGUACUUCGAAAAGCCUAGUAUUGCCUUGGA | NIE0811460 |
| | AUCUUCGACGCGUUGCGCUCAGCACCCGACCCCGGGGUGUAGCUUAGGCUGAUGAGUCUGGACAUUC | |
| | CUCACCGGUGACGGUGGUCCAGGCUGCGUUGGCGGCCUACCUAUGGCUAAUGCCAUAGGACGCUAGA | |
| | UGUGAACAAGGUGUGAAGAGCCUAUUGAGCUACAUAAGAGUCCUCCGGCCCCUGAAUGCGGCUAAUC | |
| | CUAACCACGGAGCAGGCGGUCGCAAACCAGUGACUAGCUUGUCGUAACGCGCAAGUCUGUGGCGGAA | |
| | CCGACUACUUUGGGUGUCCGUGUUUCCUGUUAUUUUUAUUAUGGCUGCUUAUGGUGACAAUCAGAGA | |
| | UUGUUAUCAUAAAGCGAAUUGGAUUGGCCAUCCGGUGAGUGUUGUGUCAGGUAUACAACUGUUUGUU | |
| | GGAACCACUGUGUUAGUUUAACCUCUCUUUCAACCAAUUAGUCAAAAACAAUACGAAGAUAGAACAA | |
| | CAAUACUACA | |
| 222 | GGGGUUGUUCCCACCCCAGAGGCCCACGUGGCGGCUAGUACUCCGGUAUUACGGUACCCUUGUACGC | Human- |
| | CUGUUUUAUACUCCCCUCCCCGUAACUUAGAAGAAACAAAAUAAGUUCAAUAGGAGGGGGUACAAAC | poliovirus- |
| | CAGUACCACCACGAACAAGCACUUCUGUCUCCCCGGUGACAUUGCAUAGACUGUCCCCACGGUUGAA | 1-strain- |
| | AGCAAUUGAUCCGUUACCCGCUCUUGUACUUCGAGAAGCCUAGUACCAUCUUGGAAUCAUCGAUGCG | EQG1419328 |
| | UUGCGCUCCACACUCAGUCCCAGAGUGUAGCUUAGGCUGAUGAGUCUGGACAUUCCUCACCGGCGAC | |
| | GGUGGUCCAGGCUGCGUUGGCGGCCUACCUGUGGCCCAAAGCCACAGGACGCUAGAUGUGAACAAGG | |
| | UGUGAAGAGCCUAUUGAGCUAUAAGAGAGUCCUCCGGCCCCUGAAUGCGGCUAAUCCCAACCACGGA | |
| | UCAAGGGUGCACGAACCAGUGUAUACCUUGUCGUAACGCGCAAGUCCGUGGCGGAACCGACUACUUU | |
| | GGGUGACCGUGUUUCCUUUUAUUAUUUCAAUGGCUGCUUAUGGUGACAAUCAUUGAUUGUUAUCAUA | |
| | AAGCGAAUUGGACUGGCCAUCCGGUGAAAGUGAAACAUAUUGUUUGCCUCCUCGUUGGGUCUACUUC | |
| | AACCAAUCUUUACUUACAAUCUUACCACUACAGUUUUGCUGGUUAGAAGUGUGUUUCACG | |
| 223 | CUGGGGUUGUACCCACCCCAGAGGCCCACGUGGCGGCUAGCACUCCGGUAUUACGGUACCCUUGUGC | Human- |
| | GCCUGUUUUAUACUCCCCUCCCGCAACUUAGAAGCACGAAACCAAGUUCAAUAGAAGGGGGUACAAA | poliovirus- |
| | CCAGUACCACUACGAACAAGCACUUCUGUUUCCCCGGUGACAUUGCAUAGACUGCUCACGCGGUUGA | 2-isolate- |
| | AAGUGAUCGAUCCGUUACCCGCUUGUGUACUUCGAAAAGCCUAGUAUCGCCUUGGAAUCUUCGACGC | IS061 |
| | GUUGCGCUCAGCACCCGACCCCGGGGUGUAGCUUAGGCCGAUGAGUCUGGACAUUCCUCACCGGUGA | |
| | CGGUGGUCCAGGCUGCGUUGGCGGCCUACCUAUGGCUAACGCCAUAGGACGUUAGAUGUGAACAAGG | |
| | UGUGAAGAGCCUAUUGAGCUACAUAAGAGUCCUCCGGCCCCUGAAUGCGGCUAAUCCUAACCACGGA | |
| | GCAGGCGGUCGCGAACCAGUGACUGGCUUGUCGUAACGCGCAAGUCUGUGGCGGAACCGACUACUUU | |
| | GGGUGUCCGUGUUUCCUGUUAUUUUUAUCAUGGCUGCUUAUGGUGACAAUCAGAGAUUGUUAUCAUA | |
| | AAGCGAAUUGGAUUGGCCAUCCGGUGAGUGUUGUGUCAGGUAUACAACUGUUUGUUGGAACCACUGU | |
| | GUUAGCUUUGCUUCUCAUUUAACCAAUUAAUCAAAAACAAUACGAGGAUAAAACAACAAUACUACA | |
| 224 | UUAAAACAGCCUGUGGGUUGUACCCACCCACAGGGCCCACUGGGCGCUAGCACUCUGGUAUCACGGU | Coxsackievir- |
| | ACCUUUGUGCGCCUGUUUUAUGCCCCCUUCCCCCAAUUGAAACUUAGAAGUUACACACACCGAUCAA | B5 |
| | CAGCGGGCGUGGCAUACCAGCCGCGUCUUGAUCAAGCACUCCUGUUUCCCCGGACCGAGUAUCAAUA | |
| | GACUGCUCACGCGGUUGAAGGAGAAAACGUUCGUUACCCGGCUAACUACUUCGAGAAACCUAGUAGC | |
| | AUCAUGAAAGUUGCGAAGCGUUUCGCUCAGCACAUCCCCAGUGUAGAUCAGGUCGAUGAGUCACCGC | |
| | AUUCCCCACGGGCGACCGUGGCGGUGGCUGCGUUGGCGGCCUGCCUACGGGGCAACCCGUAGGACGC | |
| | UUCAAUACAGACAUGGUGCGAAGAGUCGAUUGAGCUAGUUAGUAGUCCUCCGGCCCCUGAAUCCGGC | |
| | UAAUCCUAACUGCGGAGCACAUACCCUCAACCCAGGGGGCAUUGUGUCGUAACGGGUAACUCUGCAG | |
| | CGGAACCGACUACUUUGGGUGUCCGUGUUUCCUUUUAUUCUUAUAAUGGCUGCUUAUGGUGACAAUU | |
| | GAAAGAUUGUUACCAUAUAGCUAUUGGAUUGGCCAUCCGGUGUCUAACAGAGCUAUUAUAUACCUCU | |
| | UUGUUGGAUUUGUACCACUUGAUCUAAAGGAAGUCAAGACACUACAAUUCAUCAUACAAUUGAACAC | |
| | AGCAAA | |
| 225 | CUGUGGGUUGUUCCCACCCACAGGGCCCACUGGGCGCCAGCACUCUGAUUCCACGGAAUCUUUGUGC | Coxsackievir- |
| | GCCUGUUUUACAACCCUUCCCCAACUUGUAACGUAGAAGUAAUACACACUACUGAUCAAUAGCAGGC | A10 |
| | AUGGCGCGCCAGUCAUGUCUCGAUCAAGCACUUCUGUUCCCCCGGACUGAGUAUCAAUAGACUGCUC | |
| | ACGCGGUUGAAGGAGAAAACGUUCGUUACCCGGCUAACUACUUCGAGAAACCUAGUAGCACCAUAGA | |
| | AGCUGCAGAGUGUUUCGCUCAGCACUUCCCCCGUGUAGAUCAGGCUGAUGAGUCACUGCAAUCCCCA | |
| | CGGGUGACCGUGGCAGUGGCUGCGUUGGCGGCCUGCCUAUGGGGCAACCCAUAGGACGCUCUAAUGU | |
| | GGACAUGGUGCGAAGAGUCUAUUGAGCUAGUUAGUAGUCCUCCGGCCCCUGAAUGCGGCUAAUCCUA | |
| | ACUGCGGAGCACAUGCCUUCAACCCAGAAGGUAGUGUGUCGUAACGGGCAACUCUGCAGCGGAACCG | |
| | ACUACUUUGGGUGUCCGUGUUUCUUUUUAUUCCUAUAUUGGCUGCUUAUGGUGACAAUCACGGAAUU | |
| | GUUGCCAUAUAGCUAUUGGAUUGGCCAUCCGGUGUCUAAUAGAGCUAUUGUGUACCUAUUUGUUGGA | |
| | UUUACUCCGCUAUCACAUAAAUCUCUGAACACUUUGUGCUUUAUAUUGAACUUAAACACCCGAAA | |
| 226 | AUUAAAGGUUUAUACCUUCCCAGGUAACAAACCAACCAACUUUCGAUCUCUUGUAGAUCUGUUCUCU | Human-XIAP |
| | AAACGAACUUUAAAAUCUGUGUGGCUGUCACUCGGCUGCAUGCUUAGUGCACUCACGCAGUAUAAUU | |
| | AAUAACUAAUUACUGUCGUUGACAGGACACGAGUAACUCGUCUAUCUUCUGCAGGCUGCUUACGGUU | |
| | UCGUCCGUGUUGCAGCCGAUCAUCAGCACAUCUAGGUUUCGUCCGGGUGUGACCGAAAGGUAAG | |
| 227 | UUAAAACAGCCUGGGGGUUGUACCCACCCCUGGGGCCCACGCGGCGCCAGUACUCUGGUACGCUAGU | iBEV1 |
| | ACCUUUGUACGCCUGUUUUCCCCACCCUUAAAUAAAUUAAGAUUACCACUGCUGUGGGGAGUAGUCC | |
| | GACUCCGCACCGAUACGUCGCACCAGUGAACUGGUUCGCUUAGAACCUUUGCACGGAGCAGCUGGUA | |
| | UCCCCCCCCGUAACUUAGAAGCCUGGACAAACCGACCAAUAGAGGCGUUGUAGCCAGCAGCGCAACG | |
| | GUCAAGCACUUCUGUUUCCCCGGCGCCAUGCGUCGUUACCCGCCCGGCCUACUGCGGGAAGCCUAGU | |
| | AGGACCCCAAUCGGACGCGCGGUUGCGUUCAGCCACAACCCCAGUGGUAGCUCUGAGGAAUGGGACU | |
| | CGCCUACCCCCCACAGCAAUGUGGUGGCUUGUCCGCGUGUGUCCUCGGGUUCGCUCGUUGAGCGAUC | |
| | ACCGCAACUCUGAGUAAGGUCUCAAGAGCCUACUGCGCUAGGUCGGUUCUCCUCCGGAGCCGUGAAU | |
| | GCUGCUAAUCCCAACCUCCGAGCGUGUGCGCACAACCCAGUGUUGCUACGUCGUAAUGCGUAAGUUG | |
| | GAGGCGGAACAGACUACUUUCGGUACUCCGUGUUUCCUUAAUUUUUGUUCAUUAUUACAUGGUGACA | |
| | UUGACUGACAUUUGUGAAUUUGCCCGCCUGCCCUUGAACAUAGCUCUCUAUUACUUGAUUGCAUUUC | |
| | ACAAAACAACCCAACUCACUGUUGAACUUGUUGACUUCGCAGUCUACUUGAACUUACAGUACAAUCA | |
| | CAUUCACA | |
| 228 | CUUGUAAAAACAACUUUGAUGCCUUGAAUAUAUAAUGAUUCAUUAUAACAAUUAUGCAUAGAUUUUA | iCovid19 |
| | AUAAUCUGCAUAUUUUAUGCUUUCAUGUUUUUCCUAAUUAAUGAUUUGACAUGGUUAAUAAUUAUAA | |
| | UAUAUUCUGCAUCACAGUUUACAUAUUUAUGUAAAAUAAGCAUUUAAAAAUUAUUAGUUUUAUUCUG | |
| | CCUGCUUAAAUAUUACUUUCCUCAAAAAGAGAAAACAAAAAUGCUAGAUUUUACUUUAUGACUUGAA | |
| | UGAUGUGGUAAUGUCGAACUCUAGUAUUUAGAAUUAGAAUGUUUCUUAGCGGUCGUGUAGUUAUUUU | |
| | UAUGUCAUAAGUGGAUAAUUUGUUAGCUCCUAUAACAAAAGUCUGUUGCUUGUGUUUCACAUUUUGG | |
| | AUUUCCUAAUAUAAUGUUCUCUUUUUAGAAAAGGUGGACAAGUCCUAUUUUCAAGAGA | |
| 229 | UUUAAUAAGUGUUGUGCAGAUUAAUCUGCACAGCACUAGCCACAAAGUUCAGUGGUCCCAAUUAUGG | iCrPV |
| | GAUAUAAAACUCAAUGGUCGACGGAGUGACCUGGAAAUGCUCCCCGUGAGAAACCUUGUUUAAACGC | |
| | ACUUGGACCUUCUCCGGCCUCAGCGCCAGGCAUAGCUGGAUCGUCAAUCCCAAGUUUGGACUUAGAC | |
| | UUGCUCUAUGUUACAAUUGGGAGGAGCGUGACUUACCAUUGGUCGUUGAUGACGUUAAUUCCAAACA | |
| | AAAACAACAACAACAACUUAGUUUAAAUAAUUCCGGUCUUUAAGCAUCAGGGUAGACCGAUGAGGAA | |
| | UAAAGAACCCUAUAAGACUGAUUCGAUACCAAGAGCUGGUGUCUGAGGGAUUACGACUCGCGUGGUC | |
| | GGACAGCACAUGUUAGUCAUGUAUUAUGGUGUUUCCCAACACGUAUCUUUGCUCUAGUAUUGAACUU | |
| | UAGUGAUGAGUGUACUUGGCAUGUACACCUGAUUAGUUAGCCCUUGUGGAUACGCCACUGCCGCUCA | |
| | GGUACAGUAGGACAUACCCCAAAUAUUGGGUUCCACUCGUAGAGAAGAAAAUUAGUGGCGAAACGUA | |
| | CGGUUUAUGUCAAAGGAGAAGGUGAUUUUAAUUGCACCUGAAGCAACCCUAGGUCUUCCCUUUGAUG | |
| | UGAGCUAAUAAGGAGUAACCGAUUCUUACAAUGUGAUC | |
| 230 | AGCGCCCAAUACGCAAACCGCCUCUCCCCGCGCGUUGGCCGAUUCAUUAAUGCAGCUGGCACGACAG | iEMCV |
| | GUUUCCCGACUGGAAAGCGGGCAGUGAGCGCAACGCAAUUAAUGUGAGUUAGCUCACUCAUUAGGCA | |
| | CCCCAGGCUUUACACUUUAUGCUUCCGGCUCGUAUGUUGUGUGGAAUUGUGAGCGGAUAACAAUUUC | |
| | ACACAGGAAACAGCUAUGACCAUGAUUACGCCAAGCUUCCAGGCACUCCUUUCAAGACCUAGAAGGU | |
| | CCAUUAGCUGCAAAGAUUCCUCUCUGUUUAAAACUUUAUCCAUCUUUGCAAAGCUUUUUGCAAAAGC | |
| | CUAGGCCUCCAAAAAAGCCUCCUCACUACUUCUGGAAUAGCUCAGAGGCCGAGGCGGCCUCGGCCUC | |
| | UGCAUAAAUAAAAAAAAUUAGUCAGCCAUGGGGCGGAGAAUGGGCGGAACUGGGCGGAGUUAGGGGC | |
| | GGGAUGGGCGGAGUUAGGGGGGGGACUAUGGUUGCUGACUAAUUGAGAUGCAUGCUUUGCAUACUUC | |
| | UGCCUGCUGGGGAGCCUGGCGUGAGGCUCCGGUGCCCGUCAGUGGGCAGAGCGCACAUCGCCCACAG | |
| | UCCCCGAGAAGUUGGGGGGAGGGGUCGGCAAUUGAACCGGUGCCUAGAGAAGGUGGCGCGGGGUAAA | |
| | CUGGGAAAGUGAUGUCGUGUACUGGCUCCGCCUUUUUCCCGAGGGUGGGGGAGAACCGUAUAUAAGU | |
| | GCAGUAGUCGCCGUGAACGUUCUUUUUCGCAACGGGUUUGCCGCCAGAACACAGGUAAGUGCCGUGU | |
| | GUGGUUCCCGCGGGCCUGGCCUCUUUACGGGUUAUGGCCCUUGCGUGCCUUGAAUUACUUCCACGCC | |
| | CCUGGCUGCAGUACGUGAUUCUUGAUCCCGAGCUUCGGGUUGGAAGUGGGUGGGAGAGUUCGAGGCC | |
| | UUGCGCUUAAGGAGCCCCUUCGCCUCGUGCUUGAGUUGAGGCCUGGCCUGGGCGCUGGGGCCGCCGC | |
| | GUGCGAAUCUGGUGGCACCUUCGCGCCUGUCUCGCUGCUUUCGAUAAGUCUCUAGCCAUUUAAAAUU | |
| | UUUGAUGACCUGCUGCGACGCUUUUUUUCUGGCAAGAUAGUCUUGUAAAUGCGGGCCAAGAUCUGCA | |
| | CACUGGUAUUUCGGUUUUUGGGGCCGCGGGCGGCGACGGGGCCCGUGCGUCCCAGCGCACAUGUUCG | |
| | GCGAGGCGGGGCCUGCGAGCGCGGCCACCGAGAAUCGGACGGGGGUAGUCUCAAGCUGGCCGGCCUG | |
| | CUCUGGUGCCUGGCCUCGCGCCGCCGUGUAUCGCCCCGCCCUGGGCGGCAAGGCUGGCCCGGUCGGC | |
| | ACCAGUUGCGUGAGCGGAAAGAUGGCCGCUUCCCGGCCCUGCUGCAGGGAGCUCAAAAUGGAGGACG | |
| | CGGCGCUCGGGAGAGCGGGCGGGUGAGUCACCCACACAAAGGAAAAGGGCCUUUCCGUCCUCAGCCG | |
| | UCGCUUCAUGUGACUCCACGGAGUACCGGGCGCCGUCCAGGCACCUCGAUUAGUUCUCGAGCUUUUG | |
| | GAGUACGUCGUCUUUAGGUUGGGGGGAGGGGUUUUAUGCGAUGGAGUUUCCCCACACUGAGUGGGUG | |
| | GAGACUGAAGUUAGGCCAGCUUGGCACUUGAUGUAAUUCUCCUUGGAAUUUGCCCUUUUUGAGUUUG | |
| | GAUCUUGGUUCAUUCUCAAGCCUCAGACAGUGGUUCAAAGUUUUUUUCUUCCAUUUCAGGUGUCGUG | |
| | AGGAAUUCUCUAGGUCGACCCCGGGGAUCCUCUAGACUAGUGCUAGCAUGCUGCAGUCGAAGGUACC | |
| | GCGGGCCCGGGAUCCGCCCCUCUCCCUCCCCCCCCCCUAACGUUACUGGCCGAAGCCGCUUGGAAUA | |
| | AGGCCGGUGUGCGUUUGUCUAUAUGUUAUUUUCCACCAUAUUGCCGUCUUUUGGCAAUGUGAGGGCC | |
| | CGGAAACCUGGCCCUGUCUUCUUGACGAGCAUUCCUAGGGGUCUUUCCCCUCUCGCCAAAGGAAUGC | |
| | AAGGUCUGUUGAAUGUCGUGAAGGAAGCAGUUCCUCUGGAAGCUUCUUGAAGACAAACAACGUCUGU | |
| | AGCGACCCUUUGCAGGCAGCGGAACCCCCCACCUGGCGACAGGUGCCUCUGCGGCCAAAAGCCACGU | |
| | GUAUAAGAUACACCUGCAAAGGCGGCACAACCCCAGUGCCACGUUGUGAGUUGGAUAGUUGUGGAAA | |
| | GAGUCAAAUGGCUCUCCUCAAGCGUAUUCAACAAGGGGCUGAAGGAUGCCCAGAAGGUACCCCAUUG | |
| | UAUGGGAUCUGAUCUGGGGCCUCGGUACACAUGCUUUACAUGUGUUUAGUCGAGGUUAAAAAAACGU | |
| | CUAGGCCCCCCGAACCACGGGGACGUGGUUUUCCUUUGAAAAACACGAUGAUAAUAUGGCCACAACC | |
| 231 | UUAAAACAGCCUGUGGGUUGCACCCACUCACAGGGCCCACUGGGCGCAAGCACUCUGGUACCUCGGU | 1EV71 |
| | ACCUUUGUGCGCCUGUUUUACACCCCCCCCCCAGUGAAACUUAGAAGCAGCAAACCACGAUCAAUAG | |
| | CGGGCAUAACGCUCCAGUUAUGUCUUGAUCAAGCACUUCUGUUUCCCCGGACUGAGUAUCAAUAGAC | |
| | UGCUCGCGCGGUUGAAGGAGAAAACGUUCGUUAUCCGGCUAGCUACUUCGGGAAACCUAGUAACACC | |
| | AUGAAAGUUGCGGAGAGCUUCGUUCAGCACUCCCCCAGUGUAGAUCAGGUCGAUGAGUCACCGCAUU | |
| | CCCCACGGGCGACCGUGGCGGUGGCUGCGUUGGCGGCCUGCCCAUGGGGUAACCCAUGGGGCGCUCU | |
| | AAUACGGACAUGGUGUGAAGAGUCUACUGAGCUAGUUAGUAGUCCUCCGGCCCCUGAAUGCGGCUAA | |
| | UCCCAACUGCGGAGCACACGCCCACAAGCCAGCGGGUAGCGUGUCGUAACGGGUAACUCUGCAGCGG | |
| | AACCGACUACUUUGGGUGUCCGUGUUUCUUUUUAUCUUUAUACUGGCUGCUUAUGGUGACAAUUAAA | |
| | GAAUUGUUACCAUAUAGCUAUUGGAUUGGCCAUCCGGUGUGCAACAGAGCAAUUGUUUACCUAUUCA | |
| | UUGGUUUCGUACCAUUAACCUUGAAGUCUGUGACCACCCUUAACUACAUCUUGACCCUUAACACAGC | |
| | UAAAC | |
| 232 | GACGGAUCGGGAGAUCUUCAAUAUUGGCCAUUAGCCAUAUUAUUCAUUGGUUAUAUAGCAUAAAUCA | IHCV |
| | AUAUUGGCUAUUGGCCAUUGCAUACGUUGUAUCUAUAUCAUAAUAUGUACAUUUAUAUUGGCUCAUG | |
| | UCCAAUAUGACCGCCAUGUUGGCAUUGAUUAUUGACUAGUUAUUAAUAGUAAUCAAUUACGGGGUCA | |
| | UUAGUUCAUAGCCCAUAUAUGGAGUUCCGCGUUACAUAACUUACGGUAAAUGGCCCGCCUGGCUGAC | |
| | CGCCCAACGACCCCCGCCCAUUGACGUCAAUAAUGACGUAUGUUCCCAUAGUAACGCCAAUAGGGAC | |
| | UUUCCAUUGACGUCAAUGGGUGGAGUAUUUACGGUAAACUGCCCACUUGGCAGUACAUCAAGUGUAU | |
| | CAUAUGCCAAGUCCGCCCCCUAUUGACGUCAAUGACGGUAAAUGGCCCGCCUGGCAUUAUGCCCAGU | |
| | ACAUGACCUUACGGGACUUUCCUACUUGGCAGUACAUCUACGUAUUAGUCAUCGCUAUUACCAUGGU | |
| | GAUGCGGUUUUGGCAGUACACCAAUGGGCGUGGAUAGCGGUUUGACUCACGGGGAUUUCCAAGUCUC | |
| | CACCCCAUUGACGUCAAUGGGAGUUUGUUUUGGCACCAAAAUCAACGGGACUUUCCAAAAUGUCGUA | |
| | AUAACCCCGCCCCGUUGACGCAAAUGGGCGGUAGGCGUGUACGGUGGGAGGUCUAUAUAAGCAGAGC | |
| | UCGUUUAGUGAACCGUCAGAUCACUAGAAGCUUUAUUGCGGUAGUUUAUCACAGUUAAAUUGCUAAC | |
| | GCAGUCAGUGCUUCUGACACAACAGUCUCGAACUUAAGCUGCAGAAGUUGGUCGUGAGGCACUGGGC | |
| | AGGUAAGUAUCAAGGUUACAAGACAGGUUUAAGGAGACCAAUAGAAACUGGGCUUGUCGAGACAGAG | |
| | AAGACUCUUGCGUUUCUGAUAGGCACCUAUUGGUCUUACUGACAUCCACUUUGCCUUUCUCUCCACA | |
| | GGUGUCCACUCCCAGUUCAAUUACAGCUCUUAAGGCUAGAGUACUUAAUACGACUCACUAUAGGCUA | |
| | GCCACC | |
| 233 | UACUCUGGUAUUAUGUACCUUUGUACGCCUGUUUCUUCCCUACAACCCCUUCCUAAAACUCCCACCC | iHRV-B14 |
| | AUGAAACGUUAGAAGCUUGACAUUUAAGUACAAUAGGUGGCACCACAUCCAGUGGUGUCUACGUACA | |
| | AGCACUUCUGUUUCCCCGGAGCGAGGUAUAGGCUGUACCCACUGCCAAAAGCCUUUAACCGUUAUCC | |
| | GCCAACCAACUACGUAACAGCUAGUAUCAUCUUGUUCUUCACUGGACGUUCGAUCAGGUGGAUUCCC | |
| | CCUCCACUAGUUUGGUCGAUGAGGCUAGGAGCUCCCCACGGGUGACCGUGUCCUAGCCUGCGUGGCG | |
| | GCCAACCCAGCUCAUGCUGGGACGCCCUUUUAAGGACAUGGUGUGAAGACUCGCAUGUGCUUGGUUG | |
| | UGAGUCCUCCGGCCCCUGAAUGCGGCUAACCUUAACCCUGGAGCCUUAUGCCACGAACCAGUGGUUG | |
| | UAAGGUCGUAACGAGCAAUUCCGGGAUGGGACCGACUACUUUGGGUGUCCGUGUUUCCUAUUUUUCU | |
| | UUAUAUUGUCUUAUGGUCACAGCAUAUAUAUAAGUAUAUACUGUGAUC | |
| 234 | UUAAAACAGCUCUGGGGUUGUUCCCACCCCAGAGGCCCACGUGGCGGCCAGUACACUGGUAUCGCGG | iPV2 |
| | UACCUUUGUACGCCUGUUUUAUACUCCCUUCCCCCGUAACUUAGAAGCACAACGUCCAAGUUCAAUA | |
| | GAAGGGGGUACAAACCAGUACCACCACGAACAAGCACUUCUGUUCCCCCGGUGAGGCUGUAUAGGCU | |
| | GUUUCCACGGCUAAAAGCGGCUGAUCCGUUAUCCGCUCAUGUACUUCGAGAAGCCUAGUAUCACCUU | |
| | GGAAUCUUCGAUGCGUUGCGCUCAACACUCAACCCCAGAGUGUAGCUUAGGUCGAUGAGUCUGGACG | |
| | UUCCUCACCGGCGACGGUGGUCCAGGCUGCGUUGGCGGCCUACCUGUGGCCCAAAGCCACAGGACGC | |
| | UAGUUGUGAACAAGGUGUGAAGAGCCUAUUGAGCUACCUGAGAGUCCUCCGGCCCCUGAAUGCGGCU | |
| | AAUCCUAACCACGGAGCAGGCAGUGGCAAUCCAGCGACCAGCCUGUCGUAACGCGCAAGUUCGUGGC | |
| | GGAACCGACUACUUUGGGUGUCCGUGUUUCCUUUUAUUUUUACAAUGGCUGCUUAUGGUGACAAUUA | |
| | UUGAUAGUUAUCAUAAAGCAAAUUGGAUUGGCCAUCCGGUGAGAAUUUGAUUAUUAAAUUACUCUCU | |
| | UGUUGGGAUUGCUCCUUUGAAAUCCUGUGCACUCACACCUAUUGGAAUUACCUCAUUGUUGAGAUAU | |
| | UAUUACCACU | |
| 235 | UUAAAACAGCUCUGGGGUUGCUCCCACCCCAGAGGCCCACGUGGCGGCCAGUACUCUGGUAUUGCGG | iPV3 |
| | UACCUUUGUACGCCUGUUUUAUACUCCCUCCCCCCGUGCAACUUUAGAAGAAAUCCACAAAGUUCAA | |
| | UAGAGGGGGUGCAAACCAGCACCACCACGAACAAGCACUUCUGUUUCCCCGGUGAUGUCGUAUAAGC | |
| | UGUACCCACGGCUGAAGGCGACGGAUCCGUUAUCCGCUUGAGUACUUCGAGAAGCCUAGUAUUACCU | |
| | UGGAAUCUUCGACGCGUUGCGCUCAGCACUCUACCCCGAGUGUAGCUUAGGUCGAUGAGUCUGGGCA | |
| | CGCCCCACCGGCGACGGUGGCCCAGGCUGCGUUGGCGGCCUACCCAUGGCUAUCACCAUGGGACGCU | |
| | AGUUGUGAACAAGGUGUGAAGAGCCUAUUGAGCUACCCAAGAGUCCUCCGGCCCCUGAAUGCGGCUA | |
| | AUCCUAACCACGGAGCAAGUGUCCUCAACCCAGGGGAUGGCUUGUCGUAACGCGAAAGUCUGUGGCG | |
| | GAACCGACUACUUUGGGUGUCCGUGUUUCCUUUUAUUUUUAUGUAUGGCUGCUUAUGGUGACAAUCA | |
| | AAGGUUGUUACCAUAAAGCAAUUUGGAUUGGCCAUCCGGUGAGAAUCAAACAUAUUAUCUACCUGUU | |
| | UGUUGGGUUUUCUUCUUUUACUUGAACAAUACCUCUAAUAAUAACUGCUAUAUUGUCAAUAAGACAU | |
| | UAUCAUCACA | |
| 236 | GAGUGUUCCCACCCAACAGGCCCACUGGGUGUUGUACUCUGGUAUUACGGUACCUUUGUACGCCUAU | iSimianEV-A |
| | UUUAUUUCCCCCCCCUUUUUGAAACUUAGAAGUUAAUAAUAAACACGCUCACUAGGUGCACUACAUC | |
| | CAGUAGUGUAAUGAGCAAGCACUUCUGUCUgCCCCGGGAGGGAUAUAUGGUACGCUGUGCAAACGGC | |
| | GGAAAUUAAUCCUACCGUUAACCGCCCACCUACUCCGAGAAGCCUAGUACCUAAUUGGAUUUAUCAA | |
| | UGGAGUUGCGCUCAGCAGGUGACCCUGACCUGCCAGCUCCGGCUGAUGGACCUGGGCUUUCCCCACA | |
| | GGCGACUGUGGCCCAGGUCGCGUGGCGGCCGGCCCACCCCCCUGGGUGGGACGCCUUGAUAAUGACA | |
| | AGGUGGGAAGAGCCUAUUGGGCUAGCUGGUUUCCUCCGGCCUCCUGAAUGCGGCUAACCUUAACCCC | |
| | AGAGCAUAUGGUAGCAACCCAGCUACUAGUAUGUCAUAAUGCGUAAGUCUGGGAUGGGACCGACUAC | |
| | UUUGGAGAGUCCGUGUUUCUAUUGUUUCUUUAAUCAAUCUUAUGGUGACAAUUUAUAGUGCCCUGAG | |
| | UAUUGAUUGGUUGUUGCUUUUGACAAUUAUUGAGACAUCACAUAGACAUA | |
| 237 | UUAAAAUAGCUGCUGGGUUGUUCCCAUCCAGCAGGCCCACUGGGCGUGAUACUCUGGUAUUGCGGUA | iSimianV4 |
| | CCUUUGUAUGCCUAUUUUACCUCCCUUCCCCCUGAAACUUAGAAGAAAGAAACAAACACGCUCACUA | |
| | GGUACACUGCAUCCAGCAGUGUAAUGAGCAAGCACUUCUGUUUCCCCGGAAGGGAUAUAUGGUACGC | |
| | UGUGCAAACGGCGGAAAUUAAUCCUACCGUUAACCGCCCAUCUACUCCGAGAAGCCUAGUACCUAAU | |
| | UGAACUUAUCAAUGGAGUUGCGCUCAGCAGGUGACCCUGACCUGCCAGCUCCGGCUGAUGGACCUGG | |
| | GCAUCCCCCACAGGCGACUGUGGCCCAGGUCGCGUGGCGGCCGGCCCACUCUUUUGAGUGGGACGCC | |
| | UUGAUAAUGACAAGGUGGGAAGAGCCUAUUGGGCUAGCUGGUUUCCUCCGGCCUCCUGAAUGCGGCU | |
| | AACCCUAACCCCGGAGCAUAUGGUGGCAACCCAGCCACUGGUAUGUCGUAACGCGUAAGUCUGGGAU | |
| | GGGACCGACUACUUUGGAGAGUCCGUGUUUCCAUUAUUUUCUUUAUUGAAUCUUAUGGUGACAAAUU | |
| | GGGAGUUACUCGGGUGACGAUUGAUUAUUACUUCUGAAAACGUAGAGAUAUAGAAUCAACACA | |
| 238 | UUAAAACAGCCUGUGGGUUGUUCCCACCCACAGGGCCCACUGGGCGCUAGCACACUGGUAUCACGGU | iSwineVesicular |
| | ACCUUUGUGCGCCUGUUUGACUUACCCUCCCCAAACGCAACUUAGAAGCACAACUUAAAUGGUCAAU | |
| | AGGCGGCUCAGUAUGCCAACUGAGUCUCGAUCAAGCACUUCUGUUACCCCGGACUGAGUACCAAUAG | |
| | GCUGCUCACGCGGCUGAAGGGGAAACCGUUCGUUACCCGACUAACUACUUCGAGAAACCUAGUACCA | |
| | CCAUGAAAGUUGCGCAGCGUUUCGCUCCGCACAACCCCAGUGUAGAUCAGGUCGAUGAGUCACCGCA | |
| | AACCCCACGGGCGACCGUGGCGGUGGCUGCGCUGGCGGCCUGCCCAUGGGGCAACUCAUGGGACGCU | |
| | UCAAUACUGACAUGGUGCGAAGAGUCUAUUGAGCUAGUUGGUAGUCCUCCGGCCCCUGAAUGCGGCU | |
| | AAUCCUAACUGCGGAGCAGAUACCCACGCACCAGUGGGCAGUCUGUCGUAAUGGGCAACUCUGCAGC | |
| | GGAACCGACUACUUUGGGUGUCCGUGUUUCCUUUUGUUCUUAUACUGGCUACUUAUGGUGACAAUUG | |
| | AGAGAUUGUAACCAUAUUGCUAUUGGAUUGGCCACCCGGCGACGAAUAGAACAGUUGCUUACCUGUU | |
| | UGUUAGUCUCGUAUCACUGAACUACAAAUCCUUAAACACCCUUUAAUUUCGUCAUAACGCUCAAUAC | |
| | GUUAAA | |
| 239 | CCCCUCUCCCUCCCCCCCCCCUAACGUUACUGGCCGAAGCCGCUUGGAAUAAGGCCGGUGUGCGUUU | cardiovirusA1; |
| | GUCUAUAUGUUAUUUUCCACCAUAUUGCCGUCUUUUGGCAAUGUGAGGGCCCGGAAACCUGGCCCUG | encephalo- |
| | UCUUCUUGACGAGCAUUCCUAGGGGUCUUUCCCCUCUCGCCAAAGGAAUGCAAGGUCUGUUGAAUGU | myocarditisvirus1 |
| | CGUGAAGGAAGCAGUUCCUCUGGAAGCUUCUUGAAGACAAACAACGUCUGUAGCGACCCUUUGCAGG | |
| | CAGCGGAACCCCCCACCUGGCGACAGGUGCCUCUGCGGCCAAAAGCCACGUGUAUAAGAUACACCUG | |
| | CAAAGGCGGCACAACCCCAGUGCCACGUUGUGAGUUGGAUAGUUGUGGAAAGAGUCAAAUGGCUCUC | |
| | CUCAAGCGUAUUCAACAAGGGGCUGAAGGAUGCCCAGAAGGUACCCCAUUGUAUGGGAUCUGAUCUG | |
| | GGGCCUCGGUGCACAUGCUUUACAUGUGUUUAGUCGAGGUUAAAAAACGUCUAGGCCCCCCGAACCA | |
| | CGGGGACGUGGUUUUCCUUUGAAAAACACGAUGAUAA | |
| 240 | UUGAAAGGGGGCCCGGGGGAUCUCCCCCGCGGUAACUGGUCACAGUUGCCGCGGACGGAGAUCAUCC | cardiovirusB1; |
| | CCCGGUUACCCCCUUUCGACGCGGGUACUGCGAUAGUGCCACCCCAGUCCUUCCUACUCCCGACUCC | Theiler's |
| | CGACCCCAACCCAGGUUCCUUGGAACAGGAACACCAAUUUAUUCAUCCCUUGGAUGCUGACUAAUCA | murineencephalo- |
| | GAGGAACGUCAGCAUUUUCCGGCCCAGGCUAAGAGAAGUAGAUAAGUUAGAAUCUAAAUUAUUUAUC | myeltitsvirus- |
| | AUCCCCUUGACGAAUUCGCGUUGGAAAAGCACCUCUCACUUGCCGCUCUUCACACCCAUCAUUCUAA | GD |
| | UUCGGCCCCUGUGUUGAGCCCCUUGUUGAAGUGUUUCCCUCCAUCGCGACGUGGUUGGAGAUCUAAG | |
| | UUAACCGACUCCGACGAAACUACCAUCAUGCCUCCCCGAUUAUGUGAUGCUUUCUGCCCUGCUGGGU | |
| | GGAGCAUCCUCGGGUUGAGAAAUCUUUCUUCCUUUUACCUUGGACUCCGGUCCCCCGGUCUAAGCCG | |
| | CUUGGAAUAAGACAGGGUUAUCUUCACUCCUCUUCUUUUCUACUUCACAGUGUUCUAUGCUGUGAAA | |
| | GGGUAUGUGUCGCCCCUUCCUUCUUCGGAGAACACGCGUGGCGGUUUUUUCCGUCUCUCGACAAGCG | |
| | CGUGUGCGACAUGCAGAGUCUCGCGAAGAAAGCAGUUCUCGGUCUAGCUUUAGUGCCCACAAGAAAA | |
| | CAGCUGUAGCGACCACACAAAGGCAGCGGAACCCCCCUCCUGGUAACAGGAGCCUCUGCGGCCAAAA | |
| | GCCACGUGGAUAAGAUCCACCUUUGUGUGCGGUGCAACCCCAGCACCCUGUUUUCUUGGUGACACUC | |
| | UAGUGAACCCCUGAAUGGCGAUCUUAAGCGCCUCUGUAGGGAAGCCAGGAAUGUCCAGGAGGUACCC | |
| | CUUCCGCUCGGAAGGGAUCUGACCUGGAGACACAUCACAUGUGCUUUACACCUGUGCUUGUGUUUAA | |
| | AAAUUGUCGCAGCUUCCCCAAACCAAGUGGUCUUGGUUUUCUCUUUUUAUUAUAUUGUCAAU | |
| 241 | UUGAAAGGGGGCCCGGGGGAUCUCCCCCGCGGCAACUGGUUACAGUUGUCGCGGACGGAGAUCAUCC | cardiovirusB1; |
| | CCCGGUCACCCCCUUUCGACGCGGGUACUGCGAUAGUGCCACCCAGUCUCUCCUACUCCCGACUCCC | Theiler's |
| | GACCCUAACCCAGGUUCCUCGGUAUAGGAACACCACUUUACUCAUCCCCUGGAUGCUGACUAAUCAG | murineencephalo- |
| | AGGAACGUCAGCAUUUUCCGGCCCAGGCUAAGAGAAGUAGAUAAGUUACAAUCCAAAUUGAUUUAUC | omyeltitsvirus- |
| | AUCCCCUUGACGAAUUCGCGUUGGAAAAGCACCUCUCACUUGCCGCUCUUCACACCCAUCAUUUUAA | DA |
| | UUCGGCCUCUGUGUUGAGCCCCUUGUUGAAGUGUUUCUCUCCAUCGCGACGUGGUUGGAGAUCUAAG | |
| | UCAACCGACUCCGACGAAACUACCAUCAUGUCUCCCCGAUUAUGUGAUGCUUUCUGCCCUGCUGGGU | |
| | GGAGCAUCCUCGGGUUGAGAAAUCCUUCUUCCUUUCACCUUGGACCCCGGUCCCCCGGUCUAAGCCG | |
| | CUUGGAAUAAGACAGGGUUAUUUUCACCUCUUUUUCUUCUACUCCACAGUGCUCUAUACUGUGGAAG | |
| | GGUAUGUGUUGCCCCUUCCUUCUUGGAGAACGUGCGCGGCGGUCUUUCCGUCUCUCGACAAGCGCGC | |
| | GUGCAACAUGCAGAGUAACGCGAAGAAAGCAGUUCUUGGACUAGCUUUAGUAUCUGCAAGAAGACAG | |
| | CUGUAGCGACCACACAAAGGCAGCGGAACCCCCCUCCUGGUAACAGGAGCCUCUGCGGCCAAAAGCC | |
| | ACGUGGAUAAGAUCCACCUUUGUGUGCGGUGCAACCCCAGCACCCUGGUUUCUUGAAGAUACUCUAG | |
| | UGAACCCUUGAAUGGCAAUCUCAAGCGCCUCUGUAGGAAAGCCAAGAAUGUCCAGGAGGUACCCCUA | |
| | CCUCGCGGAAGGGAUCUGACCUGGAGACACGUCGCAAGUGCUUUACACCUGUGCUCGUGUUUAAAAA | |
| | UUGUCACAGCUUUCCCAAACCAAGUGGUCUUGGUUUUCCUUUUUAUUACUAUUGACACU | |
| 242 | UUGAAAGGGGGCCCGGGGGAUCUCCCCCGCGGUAACUGGUGACAGUUGUCGCGGACGGAGAUCAUCC | cardiovirusB1; |
| | CCCGGUUACCCCCUUUCGACGCGGGUACUGCGAUAGUGCCACCCCAGUCUUUCCUACUCCCGACUCC | Theiler's |
| | CGACCCUAACCCAGGUUCCUCGGAACAGGAACACCAAUUUACUCAUCCCCUGGAUGCUGACUAAUCA | murineencephalo- |
| | GAGGAACGUCAGCAUUUUCCGGCCCAGGCUAAGAGAAGUAGAUAAGUUAGAAUCCAAAUUGAUUUAU | myeltitsvirus |
| | CAUCCCCUUGACGAAUUCGCGUUGGAAAAACACCUCUCACUUGCCGCUCUUCACACCCAUUAAUUUA | |
| | AUUCGGCCUCUGUGUUGAGCCCCUUGUUGAAGUGUUUCCCUCCAUCGCGACGUGGUUGGAGAUCUAA | |
| | GUCAACCGACUCCGACGAAACUACCAUCAUGCCUCCCCGAUUAUGUGAUGCUUUCUGCCCUGCUGGG | |
| | UGGAGCACCCUCGGGUUGAGAAAACCUUCUUCCUUUUUCCUUGGACUCCGGUCCCCCGGUCUAAGCC | |
| | GCUCGGAAUAUGACAGGGUUAUUUUCACCUCUUCUUUUCUACUCCACAGUGUUCUAUACUGUGGAAG | |
| | GGUAUGUGUUGCCCCUUCCUUCUUGGAGAACGUGCGCGGCGGUCUUUCCGUCUCUCGACAAGCGCGC | |
| | GUGCAACAUACAGAGUAACGCGAAGAAAGCAGUUCUCGGUCUAGCUCUAGUGCCCACAAGAAAACAG | |
| | CUGUAGCGACCACACAAAGGCAGCGGAACCCCCCUCCUGGUAACAGGAGCCUCUGCGGCCAAAAGCC | |
| | ACGUGGAUAAGAUCCACCUUUGUGUGCGGUGCAACCCCAGCACCCUGGUUUCUUGGUGACACUCUAG | |
| | UGAACCCCUGAAUGGCAAUCUCAAGCGCCUCUGUAGGGAAGCCAAGAAUGUCCAGGAGGUACCCCUU | |
| | CCUCUCGGAAGGGAUCUGACCUGGAGACACAUCACACGUGCUUUACACCUGUGCUUGUGUUUAAAAA | |
| 243 | UUGUUACAGCUUCCCCGAACCAAGUGGUCUUGGUUUUCACUUUUUAUCACACUGUCAAU | |
| | CGCAGUACCCGCGUUGAAAGAGGGAGGGCCGGGGAUGAUCUCCACCGCGACAACUGUAGCCAGUUGC | cardiovirusB3 |
| | CGCGGGAGAGAUCCCCGGGGCCCCCUUUCAACGCGGGUACUGCGAUAGUGCCACCCCAGCCUUAUCC | |
| | UUCUCCCGACUUCCCGACCCUAACCCAGGUUCCUCGGAAAGGAACACCAAAACUCAAUCCCCUGGAU | |
| | GCUGACUAAUCAGAGGAAAGUUGGCAUUUUCCGGCCCAGGCUUAGAGAAGUAGAUAAGUUAGACUCA | |
| | AAUUGAUUCAAUUCCCCUUGGCGAAUCCGCGUCGAGGCGCCACCUCUCACUUGCCGUCCUCCACACC | |
| | CAUCCUUAAUUCGGCCUCUGUGUUGGACCCCUUGUUGAAGUGGCUUCCCUCUGCGCGGCGCGGUUGG | |
| | AGAAUUUUGUCAUCCGACUCCGACGAAACUACCAUCAUGCCUCCCCGAUUGUGUGAUGCCCUCCGCC | |
| | CUGCUGGGUGGAGCAUCCUCGGGUUGAGAAACCUCUUCCUUCAACCUUGGACCCCGGUCCCCCGGUC | |
| | UAAGCCGCUUGGAAUAAGACAGGGUUAUUUUCACUCUCUUCUUCUUCUACUCCACGGAUUUCUAUUC | |
| | CCUGGAAGGGUAUGUGUUGCCCCUCGCUUCUUGCGGAACGCGCACGGUGGUCUUUCCACCUCUCGAA | |
| | AAGUGUGCGUGCAACAUGCAGAGAUACGUGAAGAAAGCAGUUUGUGAACUAGUCCUAGAGCGAACAC | |
| | GAAUCCGGCUGUAGCGACCACUUAAGAGCAGCGGAACCCCCCUCCCGGCGACGGGAGCCUCUGCGGC | |
| | CAAAAGCCACGUGGAUAAGAUCCACUCUUGAGUGCGGUGCGGCCCCGGCACCCCGGUUUCGUGGUUG | |
| | UUCUUUGACGAACCCACAAAUGGCGAUCUUAAGCGCCUCUGUAGAGAAGCCAAGAAUGUCCAGAAGG | |
| | UACCCCUAUCAAGGGAUCUGAUCUGGAGACGUGUCACUUAUGCUUUACACGAGUGCUCGCGUUUAAA | |
| | AACUGUCACAGCUUCUCCAAACCAAGUGGUCUUGGUUUUCUCCUUCUAAUUCUCUGUCAAU | |
| 244 | GGAGAUCAUCCCCCGGUUACCCCCUUUCGACGCGGGUACUGCGAUAGUGCCACCCCAGUCUUUCCUA | cardiovirusB5 |
| | CUCCCGACUCCCGACCCUAACCCAGGUUCCUCGGAACAGGAACACAAAUUUACUCAUCCCCUGGAUG | |
| | CUGACUAAUCAGAGGAACGUCAGCAUUUUCCGGCCCAGGCUUAGAGAAGUAGAUAAGUUAGAAUCUA | |
| | AAUUGAUAUGACUUCCCCUUGACGAAUUCACGUUGGAAAUGCACCCCUCACUUGCCGCUCUUCACAC | |
| | CCACUAAUUGAUUCGGCCUACUGUGUUGAGCCCCUUGUUGAAGUGCUUCCCUCCCUCGUGACGUGGU | |
| | UGGAGAAAUCUUGUCACCCGACUCCGACGAAACUACCAUCAUGCCUCCCCGAUUAUGUGAUGCUUUC | |
| | UGCCCUGCUGGGUGGAGUAUCCUCGGGUUGAGAAAUCCUUCUUCCUUUUACCUUGGACCUUGGUCCC | |
| | CCGGUCUAAGCCGCUUGGAAUAAGACAGGGUUAUUUUCACCUCUUCUUCUUCUACUUCAUGGUGCUC | |
| | UAUACCAUGAAAGGGUAUGUGUCGCCCCUUCCUUCUUGGAGAACUCACGCGGCGGUCUUCCGUCUCU | |
| | CAAAAAGCGCGAGUGCGACAUGCAGAGUAACGCGAAGAAAGCAGUUCCUGGCCUAGCUCUAGUGCCC | |
| | ACAAGAAAACGGCUGUAGCGACCACACAAAGGCAGCGGAACUCCCCUCCUGGUAACAGGAGCCUCUG | |
| | CGGCCAAAAGCCACGUGGAUUAGAUCCACCUUUGUGUGCGGUGCAACCCCAGCACCCCGGUUUCUUG | |
| | UUGACACUCUAGUGAAUCCUUGAAUGGCAAUCUCAAGCGCCUCUGUAGGAAAGCCAAGAAUGUCCAG | |
| | GAGGUACCCCUUCCUCGCGGAAGGGAUCUGACCUGGAGACACAUCACACGUGCUUUACACUUGUGCU | |
| | UGUGUUUAAAAAUUGUCACAGCUUUCCCAAACCAAGUGGUCUUGGUUUUCCUUUUUUAUCCUACUGU | |
| | CAAU | |
| 245 | GGCAUACCUCAACCUGAGCGGCGGCUAAGGAUGCCCUGAAGGUACCCAUGAUGAAAUCGCUCUGGCG | cardiovirusC2; |
| | ACCAUGGAUCUGAUUAGGGGCCCUGCCUGGAGUGGAUCUAUCCCACACAGCGUAGGGUUAAAAAACG | Boonecardiovirus2 |
| | UCGAACCGCCCCACAAUGACCCCGGCAGGGAUGCCGGUUUUCUCUUUACCAAAUCUGACACU | |
| 246 | GGCCUAGUCGCGUGCGGUUCCCUCUUGUUUUUGCUAUGCAAUCGGGGAUGACACCCCCUUUCACGUG | cardiovirusC3 |
| | GGUACUACGAAAGUGCCCCUCGCUCCGAGGUUAAAGGAGAACCCCCCCUUCUUACCCCCACUCAGCU | |
| | CGCCCUUCAGUGUGGGCGCUAGCCUUUCCACUUGCAGCUUCUGCUUGUAGAUGCUUGCACCGUGAUC | |
| | GGUGCGCUUCUUGCUUUAGUCGCUUGUGCUUCUAUCGUUCUGACGAUUCAGUUUCCUAACGCCAGUG | |
| | UUUCGACGGCCCAAGGGGGUAGUUGCGGUCAGUAUUCCUACCGCAAUUAUCCCUUUCCCCGCUCGUA | |
| | GCUGGUUUGGAUCUUGGAUCUCUCUCCUUCCUUCCCCCGUCUUCAAUUUAGCUUCGUGAUUGAAGCA | |
| | UCUCACUGUCUCUAGUAUUUAUGUCGAACUGACGAUUGAGUACGUUCAGAUUGUGUUUGGGAGGCCC | |
| | AAGGGACCGAUGGACAACACUUCGAAAGAGUCACUUGUCCACCGCUCCUUUCCCCUACCCUAGCAAC | |
| | UCUGGAUUUGCUCACGUGGAGUUCGAGGUCUAUACUUUAACUCUGACUUGCUUUUCUUACCUUGCUA | |
| | UCUUGCUGACGUGGAUUGGUUGUAGACUGGUUCACGUUCCCGUUAGAUGCUGACGUGGAGUACGAUC | |
| | GCUGUACAUUCCACUACUGCCAAUUAGCUCCCCCUUCCCGUUGCUCCCCUCUAUAAGGAGAGCCUUC | |
| | UCUUGCAAAGGUGAAGCCUUCACCCCCGGUCGAAGCCGCUUGGAAUAAGACAGGGUUAUUUUCUCCU | |
| | CUUCUCGGCGCUUGCCUUUUCUAAGCUGAAUAGGUCCUAUCUAUUCAGGCGGAUGGUCUGGUCCGUU | |
| | CCUCCUUGGACAGAGUGUGUAUCUGGGUUUUCCGGAUCUCGACCGCACACUCACCAGAGCUCAGGAG | |
| | UGACUAAGUCAAGGCCCGAUCUGCGGCGAAAAGGAAAUGAAGUAUUUUGCAGCUGUAGCGACCUCUC | |
| | AAGGCCAGCGGAUCUCCCCACCUGGUAACAGGUGCCUCUGGGGCCAAAAGCCACGUGUUAAUAGCAC | |
| | CCUUGAGAGCGGUGGUACCCCACCACCCUGCAAACUAUGGAUUUGACUUAGUAACCAAAAGAUUGAC | |
| | UUGGCAUACCUCAACCUGAGCGGCGGCUAAGGAUGCCCUGAAGGUACCCGUGUUGAAAUCGCUUCGG | |
| | CGACCAUGGAUCUGAUCAGGGGCCCUGCCUGGAGUGGUUCUGUCCCACACAGCGUAGGGUUAAAAAA | |
| | CGUCUAACCGCCCCACAAUGACCCCGGCAGGGAUGCCGGUUUCCUUUUUACCAAAUCUUGACACU | |
| 247 | UUUCAAAGGGGGCCCUGGGGUCUUUGUCGCGGAGAUGCGCUAGCAUCUUCGCGGUAAAGAACACCCC | cardiovirusD1; |
| | AGGGGUAACCCCUUUGAACGCGGGACUGCGAUAGUGCCACCCCACGUCUUUCCCACUCCUAACCCGA | Saffoldvir1 |
| | CUCCCACCUCGGGUCCAAUGAAAGGACCAAAUAAUUCAUCCCUUGAAUGUUGGCUAAUCAGAGGAAA | |
| | GUCAGCAUUUUCCGGCCCAGGCUAAGAGAAACAUAUAAGUUAGAUUUGAUUCAAAUCCCUUGACGAA | |
| | UUCGAAACGAGAUGUUCUCCCUCCCUUGCCGCUUGUUCACACCCAUACAUUUAAUUCGGCCUCCUGU | |
| | GACAAGCCCCUCGGUGAAAGAACCUCUCUCUUUUCGACGUGGUUGGAAUUAACAUCAUUUCCGACGA | |
| | AAGUGCUAUCAUGCCUCCCCGAUUAUGUGAUGUUUUCUGCCCUGCUGGGCGGAGCAUUCUCGGGUUG | |
| | AGAAACCUUGAAUCUUUUCCUUUGGAACCUUGGUUCCCCCGGUCUAAGCCGCUUGGAAUAUGACAGG | |
| | GUUAUUUUCUUUAUCUUAUUUCUACUUUCACGGGUUCUAUCCGUGAAAAGGGUACGUGUUGCCCCUU | |
| | CCUUCUUCGGAGAAUUCACACGGCGGUCUUUCCGUCUCUCGACAAGUGUGAAUGCAACAUGCCGGAA | |
| | ACGGUGAAGAAAACAGUUUUCUGCGGAAGUCUAGAGUGCGCAUCGAAACAGCUGUAGCGACCUCACA | |
| | GUAGCAGCGGACUCCCCUCUUGGCGACAAGAGCCUCUGCGGCCAAAAGCCCCGUGGAUAAGAUCCAC | |
| | UGCUGUGAGCGGUGCAACCCCAGCACCCUGAUUCGAUGAACGUUCUUUACGGAACCAGAGGAUGGUU | |
| | UUCCUAAGCCCUCCGGUAGAGAAGCCAAGAAUGUCCUGAAGGUACCCCGCGUGCGGGAUCUGAUCAG | |
| | GAGACCAAUUGACUGUGCUUUACACCGUCACUUUGGUUUAAAAACUGUCACAGCUUCUCCAAACCAA | |
| | GUGGUCUUGGUUUUCCAAUUUUAUUAACUGGCAAU | |
| 248 | UUUGAACGCGGGACCUGCGAAAGUGCCAACCCACGUCUUUCCCACUCCUAACCCGACUCCCACCUCG | Saffoldvirus3 |
| | GGUCCAAUGAAAGGACCAAAUAUAUCAUCCCUUGAAUGCUGGCUAAUCAGAGGAAAGUCAGCAUUUU | |
| | CCGGCCCAGGCUAAGAGAAACACAUAAGUUAGAUUUGACUCAAAUCCCUUGACGAAUUCGAAACGAG | |
| | AUGUUCUCCCUCCCUUGCCGCUUGCUCACACCCACACAUUUAAUUCGGCCUCUUGUGACAAGCCCCU | |
| | UGGUGAAAGAACCUCUCUCUUUUUGACGUGGUUGGAAUUAACAUCAUUUCCGACGAAAGUGCUAUCA | |
| | UGCCUCCCCGAUUGUGUGAUGUUUUCUGCCCUGCUGGGCGGAGCAUUCUCGGGUUGAGAAACCUUGA | |
| | AUCUUUUCCUUUGGAACCUUGGUUCCCCCGGUCUAAGCCGCUUGGAAUAUGACAGGGUUAUUUUCCU | |
| | UAUCUUACUUCUACUUUCACGGGUUCUAUCCGUGAAAAGGGUACGUGUUGCCCCUUCCUUCUUCGGA | |
| | GAAUUCACACGGCGGUCUUUCCGUCUCUCGACAGGUGUGAAUGCAACAUGCCGGAAACGGUGAAGAA | |
| | AACAGUUUUCUGCGGAAGUCUUGAGUGCGCAUCGAAACAGCUGUAGCGACCUCACAGUAGCAGCGGA | |
| | CUCCCCUCUUGGUGACAAGAGCCUCUGCGGCCAAAAGCCCCGUGGAUAAGAUCCACUGCUGUGAGCG | |
| | GUGCAACCCCAGCACCCUGAUUCGAUGAACAUUCUUUACGGAACCAGAAGAUGGUUUUCUUAAGCCC | |
| | UCCGGUAGAGAAGCCAAGAAUGUCCUGAAGGUACCCCGCGUGCGGGAUCUGAUCAGGAGACCAAUUG | |
| | ACUGUGCUUUACACUGUCACUUUGGUUUAAAAACUGUCACAGCUUCUCCAAACCAAGUGGUCUUGGU | |
| | UUUCCAAUUUUACUAACUGGCAAU | |
| 249 | UUUCAAAGGGGGCCCUGGGGUCUUCGUCGCGGAGGUGCGCUAGCACCUUCGCGGCGAAGAACACCCC | Saffoldvirus4 |
| | AGGGGUACCCCCUUUGAACGCGGGACCUGCGAGAGUGCCAACCCACGUCUUUUCCCACUUUUACCCC | |
| | GGUUCCCACCUCGGGUCCAAUGAAAGGACCAAGCAAUUCAUCCCCUGAAUGUUGGCUAAUCAGAGGA | |
| | AAGUCAACAUUUUCCGGCCCAGGCUAAGAGAAACAUAUAAGUUAGAUUUGAUUCAAAUCCCUUGACG | |
| | AAUUCGGAACGAGAUGUUCUCCCUCCCUUGCCGCUUGUUCACACCCAUGUUUUAGAUUCGGCCUCUU | |
| | GUGACAAGCCCCUUGGUGAAAGAACCUCUCUCUUUUCGACGUGGUUGGAAUUAACAUCAUUUCCGAC | |
| | GAAAGUGCUAUCAUGCCUCCCCGAUUGUGUGAUGUUUUCUGCCCUGCUGGGCGGAACAUUCUCGGGU | |
| | UGAGAAACCUUGAAUCUUUUCCUUUGGAACCUUGGUUCCCCCGGUCUAAGCCGCUUGGAAUAUGACA | |
| | GGGUUAUUUUCUAAAUCUUAUUUCCACUUUUGUGGGUUCUAUCCACAAAAAGGGUACGUGCUGCCCC | |
| | UUCCUUCUUUGGAGAACUCGCACGGCGGUCUUUCCGUCUCUCAACAAGUGUGAGUGCAGCAUGCCGG | |
| | AAACGGUGAAGAAAACAGUUUCUUGUGGAAGUCUAGAGUGCACAUCGAAACAGCUGUAGCGACCUCA | |
| | CAGUAGCAGCGGACUCCCCUCUUGGCGACAAGAGCCUCUGCGGCCAAAAGCCCCGUGGAUAAGAUCC | |
| | ACUGCUGUGAGCGGUGCAACCCCAGCACCCUGAUUCGAUGGUCAUUCUCUACGGAACCAGGAAAUGG | |
| | UUUUCUUAAGCCCUCCGGCAGAGAAGCCAAGAAUGUCCUGAAGGUACCCCGCGUGCGGGAUCUGAUC | |
| | AGGAGACCAAUCGACAGUGCUUUACACUGUCGCUUUGGUUUAAAAACUGUCACAGCUUCUCCAAACC | |
| | AAGUGGUCUUGGUUUUCUAAUUUUUGUUGAAUGGCAAU | |
| 250 | UCCCUUGCCGCUUGUUCACACCCAUAUAUUUAAUUCGGCCUCUUGUGACAAGCCCCUUGGUGAAAGA | Saffoldvirus5 |
| | ACCUCUCUCUUUUCGACGUGGUUGGAAUUAACAUCAUUUCCGACGAAAGUGCUAUCAUGCCUCCCCG | |
| | AUUAUGUGAUGUUUUCUGCCCUGCUGGGCGGAGCAUUCUCGGGUUGAGAAACCUUGAAUCUUUUCCU | |
| | UUGGAACCUUGGUUCCCCCGGUCUAAGCCGCUUGGAAUAUGACAGGGUUAUUUUCUUUAUCUUAUUU | |
| | CUACUUUCACGGGUUCUAUCCGUGAAAAGGGUAUGUGUUGCCCCUUCCUUCUUCGGAGAAUUCACAC | |
| | GGCGGUCUUUCCGUCUCUCGACAAGUGUGAAUGCAACAUGCCGGAAACGGUGAAGAAAACAGUUUUC | |
| | UGCGGAAGUCUAGAUUGUGCAUCGAAACAGCUGUAGCGACCUCACAGUAGCAGCGGACUCCCCUCUU | |
| | GGCGACAAGAGCCUCUGCGGCCAAAAGCCCCGUGGAUAAGAUCCACUGCUGUGAGCGGUGCAACCCC | |
| | AGCACCCUGAUUCGAUGAACAUUCUCUACGGAACCAGAGGAUGGUUUUCUCAAGCCCUCCGGUAGAG | |
| | AAGCCAAGAAUGUCCUGAAGGUACCCCGCGUGCGGGAUCUGAUCAGGAGACCAAUUGA | |
| 251 | GAGAAACAUAUAAGUUAGAUCUGAUCCAAAUCCCUUGACGAAUUCGAAAUGAGAUGUUUUCCCUCCC | Saffoldvirus6 |
| | UUGCCGCUUGUUCACACCCAUAUUUGUGAUUCGGCCUCUUGUGACAAGCCCCUUGGUGAAAGAACCU | |
| | CUCUCUUUUCGACGUGGUUGGAAUUAACAUCUUUUCCGACGAAAGUGCUAUCAUGCCUCCCCGAUUA | |
| | UGUGAUGCUUUCUGCCCUGCUGGGCGGAGCAUUCUCGGGUUGAGAAACCUUGAAUCUUUUCCUUUGG | |
| | AACCUUGGUUCCCCCGGUCUAAGCCGCUUGGAAUAUGACAGGGUUAUUUUCUUUAUCUUAUUUCUAC | |
| | UUUCACGGGUUCUAUCCGUGAAAAGGGUACGUGUUGCCCCUUCCUUCUUCGGAGAAUUCACACGGCG | |
| | GUCUUUCCGUCUCUCGACAAGUGUGAAUGCAACAUGCCGGAAACGGUGAAGAAAACAGUUUUCUGCG | |
| | GAAGUCUAGAGUGCACAUCGAAACGGCUGUAGCGACCUCACAGUAGCAGUGGACUCCCCUCUUGGCG | |
| | ACAAGAGCCUCUGCGGCCAAAAGCCCCGUGGAUAAGAUCCACUGCUGUGAGCGGUGCAUCCCCAGCA | |
| | CCCUGAUUCGAUGAGCAUUCUCUACGGAACCAGAGAAUGGUUUUCUCAAGCCCUCCGGUAGAGAAGC | |
| | CAAGAAUGUCCUGAAGGUACCCCGCGUGCGGGAUCUGAUCAGGAGACCAAUUGA | |
| 252 | UUUCAAAGGGGGCCCUGGGGUCUUUGUCGCGGAGAUGCGCUAGCAUCUUCGCGGUGAAGAACACCCC | Saffoldvirus7 |
| | AGGGGUACCCCCUUUGGACGCGGGACCUGCGAGAGUGCCAACCCACGUCUUUUCCCAUCCCUACCCC | |
| | GACACUCACCUCGGGUCCAAUGAAAGGACCAAAUAUUUCAUCCCCUGAAUGCUGGCUAAUCAGAGGA | |
| | AAGUCAGCAUUUUCCGGCCCAGGCUAAGAGAAACAUAUAAGUUAGAUUUGACUUUAAUCCCUUGACG | |
| | AAUUCGGAACGAGAUGUUCUCCCUCCCUUGCCGCUUGUUCACACCCAUACUUUCAAUUCGACCCCUU | |
| | GUGACAAGCCCCUUGGUGAAAGAACCUCUCUCUUUUCGACGUGGUUGGAAUUAACACCAUUUCCGAC | |
| | GAAAGUGUUAUCAUGCCUCCCCGAUUAUGUGAUGCUUUCUGCCCUGCUGGGCGGAGCACUCUCGGGU | |
| | UGAGAAACCUUGAGCCUUUUCUUUUGGAACCUUGGUUCCCCCGGUCUAAGCCGCUUGGAAUAUGACA | |
| | GGGUUAUCUUCUUUAUCUUAUUUCUACUUUCACGGGGUCUAUCCGUGAAAAGGGUACGUGUUGCCCC | |
| | UUCCUUCUUCGGAGAAUUCACACGGCGGUCUUUCCGUCUCUCGACAAGUGUGAAUGCAACAUGCCGG | |
| | AACCGGUGAAGAAAACAGUUCUUUGUGGAAGUCUAGAGUGUACAUCGAAACAGCUGUAGCGACCUCA | |
| | CAGUAGCAGCGGACUCCCCUCUUGGCGACAAGAGCCUCUGCGGCCAAAAGCCCCGUGGAUAAGAUCC | |
| | ACUGCUGUGAGCGGUGCAACCCCAGCACCCUGAUUCGAUGAACAUUCUUUACGGAACCAGAGGAUGG | |
| | UUUUCUCAAGCCCUCCGGUAGAGAAGCCAAGAAUGUCCUGAAGGUACCCCGCGUGCGGGAUCUGAUC | |
| | AGGAGACCAAUUGACUGUGCUUUACACCGUCACUUUGGUUUAAAAAUUGUCACAGCUUCUCCAAACC | |
| | AAGUGGUCUUGGUUUUCCCAUUUUGUUGACUGGCAAU | |
| 253 | UUUCAAAGGGGGCCCUGGGGUCUUCGUCGCGGAGAUGCGCUAGCAUCUUCGCGGCGAAGAACACCCC | Saffoldvirus8 |
| | AGGGGUACCCCCUUUGAACGCGGGACCUGCGACAGUGCCAACCCACGUCUUUUCCUACUCCCAUCCC | |
| | GGCUCCCACCUCGAGUCCAUAGAAAGGACCAAGUACAUCAUCCCUUGGAUGCUGGCUAAUCAGAGGA | |
| | AAGUCAGCAUUUUCCGGCCCAGGCUAAGAGAAACAUAUAAGUUAGAAAUGAUCACAAUCCCUUGACG | |
| | AAAUCAGAACGAGAUGUUCUCCCUCCCUUGCCGCUUGUUCACACCCACUUCAUUAAUUCGGCCUUUU | |
| | GUGACAAGCCCCUUGGUGAAAGGACCUCUCUCUUUCUGACGUGGUUGGAAUUGACAUCUUUUCCGAC | |
| | GAAAGUGCUAUCGUGCCUCCCCGAUUAUGUGAUGUUUUCUGCCCUGCUGGGCAGAGCAUUCUCGGGU | |
| | UGAGAAACCUUGAACCUCUUCCUUUGGAACCUUGGUUCCCCCGGUCUAAGCCGCUUGGAAUAUGACA | |
| | GGGUUAUUUUCCUUAUCUUAUUUCUACUUUCAUGGGUUCUAUCCAUGAAAAGGGUAUGUGUUGCCCC | |
| | UUCCUUCUUCGGAGAAUUCACACGGCGGUCUUUCCGUCUCUCGACAAGUGUGAAUGCAACAUGCCGG | |
| | AAACGGUGAAGAAAACAGUUCUCUGUGGAAGUCUAGAGCGCGCAUCGAAACAGCUGUAGCGACCUCA | |
| | CAGUAGCAGCGGACUCCCCUCUUGGCGACAAGAGCCCCUGCGGCCAAAAGCCCCGUGGAUAAGAUCC | |
| | ACUGCUGUGAGCGGUGCAACCCCAGCACCCUGAUUCGAUGAACGUUCUUUACGGAACCAGAGGAUGG | |
| | UUUUCUCAAGCCCUCCGGUAGAGAAGCCAAGAAUGUCCUGAAGGUACCCCGCGUGCGGGAUCUGAUC | |
| | AGGAGACCAAUUGACGGUGCUUUACACCGUCACUUUGGUUUAAAAACUGUCACAGCUUCUCCAAACC | |
| | AAGUGGUCUUGGUUUUCUACUUUUAUUGACUGGCAAU | |
| 254 | UUCAAAGGGGGCCCUGGGGUCUUUGUCGCGGAGAUGCGCUAGCAUCUUCGCGGCAAAGAACACCCCA | Saffoldvirus9 |
| | GGGGUACCCCCUUUGAACGCGGGUCCUGCGAAAGUGCCAACCCAUGUCUUUCCCACUCUUUCCCCGA | |
| | CUCCCACCUCGGGUCUAAUGAAAAGACCAAAUAUUUCAUCCCUUGAAUGCUGGCUAAUCAGAGGAAA | |
| | GUCAGCAUUUUCCGGCCCAGGCUAAGAGAAACAUAUAAGUUAGAUUUGAUUUAAUCCCUUGACGAAU | |
| | UCGAAACGAGACGUUUUCCCUCCCUUGCCGCUUGUUCACACCCACUUAUUUAAUUCGGCCUUUUGUG | |
| | ACAAGCCCCUCGGUGAAAGAACCUCUCUCUUUUCGACGUGGUUGGAAUUACCAUCAUCUCCGACGAA | |
| | AGUGCUAUCAUGCCUCCCCGAUUAUGUGAUGUUUUCUGCCCUGCUGGGCGGAGCAUUCUCGGGUUGA | |
| | GAAACCUUGAAUCUUUUCCUUUGGAACCUUGGUUCCCCCGGUCUAAGCCGCUUGGAAUAUGACAGGG | |
| | UUAUUUUCUUAAUCUUAUUUCUACUUUUACGGGCUCUAUCCGUAAAAAGGGUACGUGCUGCCCCUUC | |
| | CUUCUCUGGAGAAUCUACACGGCGGUCUUUCCGUCUCUUAACAAGUGUGGAUGCAGCAUGCCGGAAA | |
| | CGGUGAAGAAAACAGUUUUCUGUGGAAGUUUAGAGUGCACAUCGAAACAGCUGUAGCGACCUCACAG | |
| | UAGCAGCGGACUCCCCUCUUGGCGACAAGAGCCUCUGCGGCCAAAAGCCCCGUGGAUAAGAUCCACU | |
| | GCUGUGAGCGGUGCAACCCCAGCACCCUGAUUCGAUGGUCAUUCUCUACGGAACCAGAAAAUGGUUU | |
| | UCUCAAGCCCUCCGGUAGAGAAGCCAAGAAUGUCCUGAAGGUACCCCGCGUGCGGGAUCUGAUCAGG | |
| | AGACCAAUUGACGGUGCUUUACACUGUCACUUUGGUUUAAAAAUUGUCACAGCUUCUCCAAACCAAG | |
| | UGGUCUUGGUUUUCCAAUUUUGUUGAGUGGCAAU | |
| 255 | UUUCAAAGGGGGGCCCUGGGGUCUUUGUCGCGGAGAUGCGCUAGCAUCUUCGCGGUAAAGAACACCC | Saffoldvirus10 |
| | CAGGGGCACCCCCUUUGAACGCGGGACCUGCGAAAGUGCCAACCCACGUCUUUCCCACUCUUAUCCC | |
| | GAUUCCCACCUCGGGUCCAAAGAAAGGACCAAUUAUUUCAUCCCUCGAAUGCUGGCUAAUCAGAGGA | |
| | AAGUCAGCAUUUUCCGGCCCAGGCUAAGAGAAACAUAUAAGUUAGAUUUGGUUCAAAUCCCUUGACG | |
| | AAUUUGGAACGAGAUGUUCUCCCUCCCUUGCCGCUUGUUCACACCCACACAUUUAAUUCGGCCUAUU | |
| | GUGACAAGCCCCUUGGUGAAAGAACCUCUCUCUUUCCAACGUGGUUGGAAUUAACAUCAUUUCCGAC | |
| | GAAAGUGUUAUCAUGCCUCCCCGAUUAUGUGAUGCUUUCUGCCCUGCUGGGCGGAGCAUUCUCGGGU | |
| | CGAGAAACCUUGAAUCUUUUCUCAUGGAACCUUGGUUCCCCCGGUCUAAGCCGCUUGGAAUAAGACA | |
| | GGGUUAUUUUCUUUAUCUUGUUUCUACUUUUAUGGGUUCUAUCCAUAAAAAGGGUAUGUGCUGCCCC | |
| | UUCCUUCUUCGGAGAAUUCACACGGCGGUCUUUCCGUCUCUCGACAGGUGUGAAUGCAGCAUGCCGG | |
| | AAACGGUGAAGAAAACAGUUUUCUGUGGAAGUCUAGAGUGUACAUCGAAACAGCUGUAGCGACCUCA | |
| | CAGUAGCAGCGGACUCCCCUCUUGGUAACAAGAGCCUCUGCGGCCAAAAGCCCCGUGGAUCAGAUCC | |
| | ACUGCUGUGAGCGGUGCAACCCCGGCACCCUGAUUCGAUGGACAUUCUCUACGGAACCAGAGAAUGG | |
| | UUUUCUCAAGCCCUCCGGUAGAGAAGCCAGGAAUGUCCUGAAGGUACCCCGCGUGCGGGAUCUGAUC | |
| | AGGAGACCAAAUAGCAGUGCUUUACGCUGUUACUUUGGUUUAAAAAUUGUCACAGCUUCUCCAAACC | |
| | AAGUGGUCUUGGUUUUCUAAUUUUAUUGAAUGGCAAU | |
| 256 | UCAAAGGGGGCCCUGGGGUCUUCGUCGCGGAGAUGCGUUAGCAUCUUCGCGGUGAAGAACACCCCAG | Saffoldvirus11 |
| | GGGUACCCCCUUUGAACGCGGGACCUGCGAGAGUGCCAACCCACGUCUAUCCCACUCCUAACCCGAC | |
| | CCCCACCUCGGGUCCUACGAAAGGACCAAAAAUUUCAUCCCUUGAAUGCUGGCUAAUCAGAGGAAAG | |
| | UCAGCAUUUUCCGGCCCAGGCUAAGAGAAACAUAUAAGUUAGAUUUGAUUCAAAUCCCUUGACGAAU | |
| | UCGAAACGAGAUGUUCUCCCUCCCUUGCCGCUUGUUCACACCCAUACACUUAAUUCGGCCUCUUGUG | |
| | ACAAGCCCCUCGGUGAAAGAACCUCUCUCUUUUCGACGUGGUUGGAAUUAACAUCAUUUCCGACGAA | |
| | AGUGCUAUCAUGCCUCCCCGAUUGUGUGAUGUUUUCUGCCCUGCUGGGCGGAGCAUUCUCGGGUUGA | |
| | GAAACCUUGAAUCUUUUCCUUUGGAACCUUGGUUCCCCCGGUCUAAGCCGCUUGGAAUAUGACAGGG | |
| | UUAUUUUCUUUAUCUUAUUUCUACUUUCACGGGUUCUAUCCGUGAAAAGGGUAUGUGUUGCCCCUUC | |
| | CUUCUUCGGAGAAUUCACACGGCGGUCUUUCCGUCUCUCGACAAGUGUGAAUGCAACAUGCCGGAAA | |
| | CGGUGAAGAAAACAGUUUUCUGCGGAAGUCUAGAGUGCGCAUCGAAACGGCUGUAGCGACCUCACAG | |
| | UAGCAGCGGACUCCCCUCUUGGUGACAAGAGCCUCUGCGGCCAAAAGCCCCGUGGAUAAGAUCCACU | |
| | GCUGUGAGCGGUGCAACCCCAGCACCCUGAUUCGAUGAACACUCUUUACGGAACCAGAGGAUGGUUU | |
| | UCUUAAGCCCUCCGGUAGAGAAGCCAAGAAUGUCCUGAAGGUACCCCGCGUGCGGGAUCUGAUCAGG | |
| | AGACCAAUUGACUGUGCUUUACACCGUCACUUUGGUUUAAAAACUGUCACAGCUUCUCCAAACCAAG | |
| | UGGUCUUGGUUUUCCAUUUUUGUUGACUGGCAAU | |
| 257 | UUUCAAAGGCGGGCCUGGGGGUGUGGGUCGCGGCACACAGUCAUGUGUGUCGCGGUCCACUCCCUCA | cardiovirusE1; |
| | GCAACCCGUCUUUGGACGUGGGUACUACGAUAGUGCCAUCCAAUUCCCCUAACCUUGUUACCUUCCG | red- |
| | AGCGACCUCCAACACCCGAGGUGCUCACCACCCUGUAAGGAAAAAUGAAAUUUCGUCUGGUGCUGGC | backedvoleca |
| | UAACAGAUAAGAUCUAGCAUUUUCCGGCCCAGGCUAAGAAACCUCUAUAAAGUAGAUCGAUAAACUU | rdiovirus |
| | AAAUUGGCGAACUGUACAAAGAACGGUCCUCUACCUUGCCGCCACACACACCCAACCCUAAAAUUUG | |
| | GCCUUUAGUGUGUGGCCCCUUGUGUAUGACCUCAACUCUUGCGUACACGCGGAAUUUAACAUCUUUC | |
| | CGCUGAAAGGAGUUUUCGUGCCUCCCCGAUUAUGUGAUGCUCGUCCUUUGCUGGGCGGAGCAACGCA | |
| | CGAAACAAGCCUUUUCUUUACUAAUGGAAUCCAAUUCCCCCGGUCUAAGCCGCUUGGAAUAAGACAG | |
| | GGUUAUUUUCACCCCCUCUUCUUCUACUCUCACAGAGCUCUAUUCUGUGAGAAGGGAAUGUUGCGCC | |
| | CCUCCUUCUUGGGAGCUCGCUUGGCGGUCUUUCCGUCUCUGUGCAAGAGCGAGUGCGCAAUGUGUUA | |
| | GAAGGUGAAGAAAGAUAUUUUGGGACUAACGUUUGACACGGAACGAAAACGUCUGUAGCGACCUCAC | |
| | AAAGGCAGCGGAACCCCCCUCCUGGUGACAGGAGCCUCUGCGGCCAAAAGCCACGUGGUUAGCAGCC | |
| | ACCUUUGUGAGCGGCGUGAACCCGACGCCACGGUUUCGUUGUGUGACACUCACUACCCCCAUUCGGU | |
| | CUUUCUUAAGCCCUAACACAGGUCCACCAAGAAUGUCCUGAAGGUACCUCUUCGGAGAUCUGAUCAG | |
| | GAGCUUCAUGCCGGUGCUUUACACCGGUUCGGAAGUUAAAAACUGUCACAGUGGACCCAAAUCUAGA | |
| | UGGUUCUAGAUUUCUAUUGGUUUUAAAAUUUCAUU | |
| 258 | CUAGACUUUCUACCCUCUUGAAUGGCGAAUUCCAGAGUAAAGUCACCUCUGCCUUGCCGCUAUACAC | cardiovirusF1; |
| | ACCCAACCAAUCGAUUCGGCCCCACGUGUAUAGCCCCUUGGUCAAGUGACCUCUUACGUCUGGACGC | grey- |
| | GGAUUUAAGACCUGAAUAUCCGCUGAAAGUUAACACCAUGUUUGCCCCGAUUAUGUGAUGUUUCUGC | backedvole- |
| | UCUUGCUGGGCGGAACAUCCGUCGGGAUGCCAAAGCAAUUUGUGUCCCCUUCUUUCUUUGGACUUCG | cardiovirus |
| | GUCCCCCGGUCGAAGCCGCUUGGAAUAAGACAGGGUUAUUUUCCCUCCUUCCUCUCUUCUACUUUGU | |
| | UAAGCUCUAUUUAACAAAAGGGUAUGUCUGGCCCCUCCCUUCUUGGGGAAUGUGCGCGGCGGUCUUU | |
| | CCGUCUCUCGAAAAGCGUGCAUGCCAGAUGCAAGAGAAGGUGAAGAAAGUAAGUUGUGUCUAACGUU | |
| | AAGACAGGAACGAAUGUGUCUGUAGCGACCUCUCAAAGGCUGCGGUCUCCCCACCUGGUAACAGGUG | |
| | CCGCAGCGGCCAAAAGCCACGUGGUUAACAGCCACCUUUGAGAGCGGUGCAACCCCAGCACCACACU | |
| | UUCGUUGUUGUUUUCUCACUAACCACAACUCGCUUCUCUGAAGCCCUCUUGUAGAGUUGCCAAGAAU | |
| | GUCCAGAAGGUACCCCGGUCCACGGGAUCUGAUCUGGAGCUCCAUGCGUGUGCUCUACACACGUUCG | |
| | GGAGUUAAAAACUGUCACAGCAACUCCAAACUAGGUGGUCCUAGUUUUCACUUUUAUUUGUCAAGUC | |
| | AUAUGGUGAUGGUUAAAGGAGAACGACA | |
| 259 | UUUAAAACAGCCUGUGGGUUGCUCCCACCCACAGGGCCCACUGGGCGCUAGCACACUGAUUUUACGG | coxsackievirA4 |
| | AAUCUUUGUGCGCCUGUUUUAUAUCCCCUCCCCCAAACAGUAAUUUAGAAGUUUAAUGCAUUUAUGG | |
| | CCAGUAGCGGGCAUGACGCACCAGUCAUGUCUUGGUCAAGCACUUCUGUUUUCCCGGACUGAGUAUC | |
| | AAUAAACUGCUCACGCGGUUGAAGGAGAAAACGUUCGUUACCCGGCUAACUACUUCGAGAAACCCAG | |
| | UAACACCAUGAAAGUUGCGGAGUGUUUCGCUCAGCACUUCCCCCGUGUAGAUCAGGUCGAUGAGUCA | |
| | CUGUAAACCCCACGGGCGACCGUGACAGUGGCUGCGUUGGCGGCCUGCCCAUGGGACAACCCAUGGG | |
| | ACGCUCUAAUACAGACAUGGUGUGAAGAGUCUAUUGAGCUAGUUAGUAGUCCUCCGGCCCCUGAAUG | |
| | CGGCUAAUCCUAACUGCGGAGCACACACCCUCAAUCCAGGGGGCAGUGUGUCGUAACGGGCAACUCU | |
| | GCAGCGGAACCGACUACUUUGGGUGUCCGUGUUUCCUUUUAUUCUUAUUGUGGCUGCUUAUGGUGAC | |
| | AAUUAAAGAAUUGUUACCAUAUAGCUAUUGGAUUGGCCAUCCGGUGUCUAACAGAGCAAUUGUGUAU | |
| | UUAUUUGCUGGAUUUGUUCCAUUAUCAUACACAUCUUUUAAUACUUUAUACUUUAUUAUAAAAUUGA | |
| | ACACAAGAAA | |
| 260 | UUAAAACAGCCUGUGGGUUGCACCCACCCACAGGGCCCACUGGGCGCCAGCACUCUGGUAUCGCGGU | coxsackievirA5 |
| | ACCUUUGUGCGCCUGUUUUAUGACCCCUCCCCAACCGAAACUUAGAAGUUACACAUACCGAUCAAUA | |
| | GUGGGCAUAGCACGCCAGCUGUGUCUUGAUCAAGCACUUCUGUUUCCCCGGACCGAGUAUCAAUAGA | |
| | CUGCUCACGCGGUUGAAGGAGAAAGCGUUCGUUAUCCGGCUAACUACUUCGAGAAACCUAGUAACAC | |
| | CAUGAAAGUUGCGGAGUGUUUCGCUCAGCACUUCCCCAGUGUAGAUCAGGUCGAUGAGUCACCGCGU | |
| | UCCCCACGGGCGACCGUGGCGGUGGCUGCGCUGGCGGCCUGCCUAUGGGAUGACCCAUAGGACGCUU | |
| | CAACACAGACAUGGUGUGAAGAGUCUAUUGAGCUAGUUAGUAGUCCUCCGGCCCCUGAAUGCGGCUA | |
| | AUCCUAACUGCGGAGCACGUGCCCUCAAGCCAGAGGGUGGCGUGUCGUAACGGGCAACUCUGCAGCG | |
| | GAACCGACUACUUUGGGAGUCCGUGUUUCCUUUUAUUCUUACACUGGCUGCUUAUGGUGACAAUUGA | |
| | GAGAUUGUUACCAUAUAGCUAUUGGAUUGGCCAUCCGGUGAGCAACAGAGCGAUUGUAUACUCUUUU | |
| | GUGGGUUUUGUGCCAUUAAAUCUUACUGUGAUUACCACACUGAAACUCAUCUUGCAGUUUAAUACAG | |
| | CAAA | |
| 261 | UUAAAACAGCCUGUGGGUUGCACCCACUCACAGGGCCCACUGGGCGCUAGCACACUGAUUUCCCGGA | coxsackievirA6 |
| | AUCCUUGUGCGCCUGUUUUAUAUCCCCUCCCCCAUGCGCAACUUAGAAGCAAUCUACACCUUCGAUC | |
| | AAUAGCAGGCGUGGCGCGCCAGCCAUGUCUAGAUCAAGCACUUCUGUUUCCCCGGACUGAGUAUCAA | |
| | UAAACUGCUCACGCGGUUGAAGGAGAAAAUGUUCGUUACCCGGCUAACUACUUCGAGAAACCUAGUA | |
| | GCACCAUGAAAAUUGCAGAGCGUUUCGCUCAGCGCUUCCCCCGCGUAGAUCAGGCUGAUGAGUCACU | |
| | GCAUUCCUCACGGGCGACCGUGGCAGUGGCUGCGUUGGCGGCCUGCCCAUGGGGUAACCCAUGGGAC | |
| | GCUCUAAUAUGGACAUGGUGUGAAGAGUCUAUUGAGCUAGUUAGUAGUCCUCCGGCCCCUGAAUGCG | |
| | GCUAAUCCUAACUGCGGAGCACAUACCCCCAAACCAGGGGGCGGUGUGUCGUAACGGGCAACUCUGC | |
| | AGCGGAACCGACUACUUUGGGUGUCCGUGUUUCCUUUUAUUCUUAUAUUGGCUGCUUAUGGUGACAA | |
| | UUGAAAGAUUGUUACCAUAUAGCUAUUGGAUUGGCCAUCCGGUGAAUAACAGAGCCUUGAUAUACCU | |
| | UUUUGUAGGGUUUAUACCACUUACUCUUCGCGUUGUUGAGACUCUAAAGUACAUUCUAAUCUUGAAC | |
| | ACUAGAAA | |
| 262 | UUUAAAACAGCCUGUGGGUUGUCCCCACCCACAGGGCCCACUGGGCGUUAGCACUCUGGUAUUACGG | coxsackievirA7 |
| | UACCUUUGUGCGCCUGUUUUAUAACCCCUCCCCAACUGUAAAUUAGAAGCAACACACGUCGAUCAAU | |
| | AGUAGGCGUGACGCACCAGUCACGUCUUGAUCAAGCACUUCUGUUUCCCCGGACCGAGUAUCAAUAG | |
| | ACUGCUCACGCGGUUGAAGGAGAAAACGUUCGUUACCCGACCAACUACUUCGAAAAACCUAGUAGCA | |
| | CCAUGAAGGUUGCGGAGUGUUUCGCUCGGCACUUCCCCAGUGUAGAUCAGGUCGAUGAGUCACCGCA | |
| | UUCCCCACGGGCGACCGUGGCGGUGGCUGCGCUGGCGGCCUGCCUAUGGGGUAACCCAUAGGACGCU | |
| | CUAAUACGGACAUGGUGCGAAGAGUCUAUUGAGCUAGUUGGUAGUCCUCCGGCCCCUGAAUGCGGCU | |
| | AAUCCUAACUGCGGAGCGCGCGCCCCCAUUCCAGGAGGUAGCGCGUCGUAACGGGCAACUCUGCAGC | |
| | GGAACCGACUACUUUGGGUGUCCGUGUUUCCUUUUAUCUUUAUUCUGGCUGCUUAUGGUGACAAUUG | |
| | ACAGAUUGUUACCAUAUAGCUAUUGGAUUGGCCAUCCGGUCACAAGCAGAGCGAUUAUCUACCAAUU | |
| | UAUUGGAUUUAUCCCACUGCACACUCGUGAAUAUAACACAUUACUGCUGAUACUGAGUUUGGACAAA | |
| | GCAAA | |
| 263 | UUAAAACAGCCUGUGGGUUGCACCCACCCACAGGGCCCAAUGGGCGUCAGCACUCUGGUACCGAGGU | coxsackievirA8 |
| | ACCUUUGUGCGCCUGUUUUAUUUCCCCUUCCCCUGAUGCAACUUAGAAGCUCCGAACUAAUGAUCAA | |
| | UAGUAGGUGUGGCACGCCAGCCACAUCUUGAUCAAGCACUUCUGUUUACCCGGACCGAGUAUCAAUA | |
| | AGCUGCGCAAGCGGCUGAAGGAGAAAGCGUUCGUUAUCCGGCCAACUACUUCGAGAAGCUUAGUACC | |
| | ACCAUGAACGUUGCAGAGUGUUUCGUUCAGCACAACCCCGGUGUAGAUCAGGCCGAUGAGUCACCGC | |
| | GUUCCCCAUGGGCGACCAUGGCGGUGGCUGCGUUGGCGGCCUGCCCAUGGAGCAAUCCAUGGGACGC | |
| | UCUAAUACUGACAUGGUGCGAAGAGCCUAUUGAGCUAGUUGGUGAUCCUCCGGCCCCUGAAUGCGGC | |
| | UAAUCCUAACUGCGGAGCAUGCGCCCACAAGCCAGUGGGUGGUGUGUCGUAACGGGUAACUCUGCAG | |
| | CGGAACCGACUACUUUGGGUGUCCGUGUUUCCUUUUAUCUUUACAUUGGCUGCUUAUGGUGACAAUU | |
| | GAAGAAUUGUUACCAUAUAGCUAUUGGAUUGGCCAUCCGGUGUGCAAUAGAGCGAUUAUAUACCUAU | |
| | UUGUUGGAUUUGUUCCAUUGACAUAUAGAUCUCUUAACACUCUACAACACAUCUUGAUCUUGAACAC | |
| | GAGAAA | |
| 264 | UUAAAACAGCCUGUGGGUUGCACCCACUCACAGGGCCCACUGGGCGCUAGCACUCUGGUACCGUGGU | coxsackievirA10 |
| | ACCUUUGUGCGCCUGUUUUAUACCCCCUUCCCCGUUUGAACAUUAGAAGUAACGCACCUCGAUCAGU | |
| | AGCAGGCGCGGCGCACCAGCCGUGUCUUGAUCAAGCACUUCUGUUUCCCCGGACCGAGUAUCAAUAG | |
| | ACUGCUCACGCGGUUGAAGGAGAAAACGUUCGUUAUCCGGCUAACUACUUCGAGAAACUUAGUAGCA | |
| | CCAUGGAAGUUGCAGAGUGUUUCGCUCAGCACUCCCCCCCCAGUGUAGAUCAGGCUGAUGGAUCACC | |
| | GCGUUCCUCACGGGUGACCGUGGCGGUGGUCGCGUUGGCGGCCUGCCCAUGGGGCAACCCAUGGGAC | |
| | GCUCUAAUAUGGACAUGGUGUGAAGAGUCUAUUGAGCUAGUUAGUAGUCCUCCGGCCCCUGAAUGCG | |
| | GCUAAUCCUAACUGCGGAGCACGUGUCCCCAACCCAGGGGAUAGCGUGUCGUAACGGGUAACUCUGC | |
| | AGCGGAACCGACUACUUUGGGUGUCCGUGUUUCCUUUAUUCUUAUCCUGGCUGCUUAUGGUGACAAU | |
| | UGAGAGAUUGUUACCAUAUAGCUAUUGGAUUGGCCAUCCGGUGUCUAACAGAGCUAUAAUAUAUCUU | |
| | UUUGUUGGGUUCGUACCCCUCAAUUUUGAGGUUGUUCUCACAUUAAAGUACAUCUUGGUGUUAAAUA | |
| | UCAGAAG | |
| 265 | UUAAAACAGCCUGUGGGUUGUACCCACCCACAGGGCCCACUGGGCGCUAGCACUCUGGUACUACGGU | coxsackievirA12 |
| | ACCUUUGUGUGCCUGUUUUAAGCCCCUACCCCCCACUCGUAACUUAGAAGGCUUCUCACACUCGAUC | |
| | AAUAGUAGGUGUGGCACGCCAGUCACACCGUGAUCAAGCACUUCUGUUACCCCGGUCUGAGUACCAA | |
| | UAAGCUGCUAACGCGGCUGAAGGGGAAAACGAUCGUUAUCCGGCUAACUACUUCGAGAAACCCAGUA | |
| | CCACCAUGAACGUUGCAGGGUGUUUCGCUCGGCACAACCCCAGUGUAGAUCAGGUCGAUGAGUCACC | |
| | GUAUUCCCCACGGGCGACCGUGGCGGUGGCUGCGUUGGCGGCCUGCCCAUGGGGUGACCCAUGGGAC | |
| | GCUCUAAUACUGACAUGGUGCGAAGAGUCUAUUGAGCUAGUUAGUAGUCCUCCGGCCCCUGAAUGCG | |
| | GCUAAUCCUAACUGCGGAGCACAUACCCUUAAUCCAAAGGGCAGUGUGUCGUAACGGGCAACUCUGC | |
| | AGCGGAACCGACUACUUUGGGUGUCCGUGUUUCCUUUUAUUCUUACAUUGGCUGCUUAUGGUGACAA | |
| | UUGAAAAGUUGUUACCAUAUAGCUAUUGGAUUGGCCAUCCGGUGACAAAUAGAGCUAUUGUAUAUCU | |
| | UUUUGUUGGUUACGUACCCCUUAAUUACAAAGUGGUUUCAACUUUGAAAUACAUCCUAACACUAAAU | |
| | UGUAGAAA | |
| 266 | UUAAAACAGCCUGUGGGUUGUACCCACCCACAGGGCCCACUGGGCGCUAGCACACUGAUUCUGCGGG | coxsackievirA14 |
| | AUCUUUGUGCGCCUGUUUUAUAUCCCCUCCCCCCAAACUAGGUAACUUAGAAGAUUAGCACUUGUAU | |
| | GACCAAUAGUAGGCGUGACGCGCCAGUCAUGUCUUGGUCAAGCACUUCUGUUUCCCCGGACUGAGUA | |
| | UCAAUAAGCUGCUUGCGCGGCUGAAGGAGAAAACGUUCGUUACCCGGCUAACUACUUCGAGAAACCU | |
| | AGUAACACCAUGAAAGUUGCGGAGUGUUUCGCUCAGCACUUCCCCCGUGUAGAUCAGGUCGAUGAGU | |
| | CACUGCAAACCCCACGGGUGACCGUGGCAGUGGCUGCGUUGGCGGCCUGCCUAUGGGUCAACCCAUA | |
| | GGACGCUCUAAUACAGACAUGGUGUGAAGAGUCUAUUGAGCUAGUUAGUAGUCCUCCGGCCCCUGAA | |
| | UGCGGCUAAUCCUAACUGCGGAGCACACGCUUGCAAACCAGUGAGUAGUGUGUCGUAAUGGGCAACU | |
| | CUGCAGCGGAACCGACUACUUUGGGUGUCCGUGUUUCCUUUUAUUCUCAUACUGGCUGCUUAUGGUG | |
| | ACAAUUGAAGAAUUGUUACUAUAUAGCUAUUGGAUUGGCCAUCCGGUGUGUAACAGAGCGAUAAUCU | |
| | ACUCCUUUGUUGCAUUUGUUCCACUAACCCUUACUACAUUAGACAUUCUACAAUACAUACUUUAUCU | |
| | CAACUCCAAGAAACGAAC | |
| 267 | UUAAAACAGCCUGUGGGUUGUACCCACCCACAGGGCCCACUGGGCGCUAGCACUCUGAUUCUACGGA | coxsackievirA16 |
| | AUCCUUGUGCGCCUGUUUUAUGUCCCUUCCCCCAAUCAGUAACUUAGAAGCAUUGCACCUCUUUCGA | |
| | CCGUUAGCAGGCGUGGCGCACCAGCCAUGUCUUGGUCAAGCACUUCUGUUUCCCCGGACCGAGUAUC | |
| | AAUAGACUGCUCACGCGGUUGAGGGAGAAAACGUCCGUUACCCGGCUAACUACUUCGAGAAGCCUAG | |
| | UAGCACCAUGAAAGUUGCAGAGUGUUUCGCUCAGCACUUCCCCCGUGUAGAUCAGGUCGAUGAGUCA | |
| | CUGCGAUCCCCACGGGCGACCGUGGCAGUGGCUGCGUUGGCGGCCUGCCUGUGGGGUAACCCACAGG | |
| | ACGCUCUAAUAUGGACAUGGUGCAAAGAGUCUAUUGAGCUAGUUAGUAGUCCUCCGGCCCCUGAAUG | |
| | CGGCUAAUCCUAACUGCGGAGCACAUACCCUCGACCCAGGGGGCAGUGUGUCGUAACGGGCAACUCU | |
| | GCAGCGGAACCGACUACUUUGGGUGUCCGUGUUUCCUUUUAUUCUUAUACUGGCUGCUUAUGGUGAC | |
| | AAUUGAAAGAUUGUUACCAUAUAGCUAUUGGAUUGGCCAUCCGGUGUGCAACAGAGCUAUUAUUUAC | |
| | CUAUUUGUUGGGUAUAUACCACUCACAUCCAGAAAAACCCUCGACACACUAGUAUACAUUCUUUACU | |
| | UGAAUUCUAGAAA | |
| 268 | UUAAAACAGCUGUGGGUUGUCACCCACCCACAGGGUCCACUGGGCGCUAGUACACUGGUAUCUCGGU | enterovirusA71 |
| | ACCUUUGUACGCCUGUUUUAUACCCCCUCCCUGAUUUGCAACUUAGAAGCAACGCAAACCAGAUCAA | |
| | UAGUAGGUGUGACAUACCAGUCGCAUCUUGAUCAAGCACUUCUGUAUCCCCGGACCGAGUAUCAAUA | |
| | GACUGUGCACACGGUUGAAGGAGAAAACGUCCGUUACCCGGCUAACUACUUCGAGAAGCCUAGUAAC | |
| | GCCAUUGAAGUUGCAGAGUGUUUCGCUCAGCACUCCCCCCGUGUAGAUCAGGUCGAUGAGUCACCGC | |
| | AUUCCCCACGGGCGACCGUGGCGGUGGCUGCGUUGGCGGCCUGCCUAUGGGGUAACCCAUAGGACGC | |
| | UCUAAUACGGACAUGGCGUGAAGAGUCUAUUGAGCUAGUUAGUAGUCCUCCGGCCCCUGAAUGCGGC | |
| | UAAUCCUAACUGCGGAGCACAUACCCUUAAUCCAAAGGGCAGUGUGUCGUAACGGGCAACUCUGCAG | |
| | CGGAACCGACUACUUUGGGUGUCCGUGUUUCUUUUUAUUCUUGUAUUGGCUGCUUAUGGUGACAAUU | |
| | AAAGAAUUGUUACCAUAUAGCUAUUGGAUUGGCCAUCCAGUGUCAAACAGAGCUAUUGUAUAUCUCU | |
| | UUGUUGGAUUCACACCUCUCACUCUUGAAACGUUACACACCCUCAAUUACAUUAUACUGCUGAACAC | |
| | GAAGCG | |
| 269 | UUUAAAACAGCCUGUGGGUUGUACCCACCCACAGGGCCCACUGGGCGCUAGCACACCGGUAUUACGG | enterovirusA76 |
| | UACCCUUGUGCGCCUGUUUUAUAUACCCUCCCCCUUGUAACUUAGAAGAAACAAAUAAACGAUCAAU | |
| | AGUAGGCUUAGCGCACCAGCUAAGUCUUGAUCAAGCACCUCUGUUACCCCGGACUGAGUAUCAAUAA | |
| | GCUGUACCCACGGCUGAAAGAGAAAACGUUCGUUAUCCGGCUAUCUACUUCGGGAAACCUAGUACCA | |
| | UCAUGAAGAUUGCAAAGCGUUUCGCUCAGCACAACCCCAGUGUAGAUCAGGUCGAUGAGUCACUGCA | |
| | UUCCCCACAGGCGACUGUGGCAGUGGCUGCGUUGGCGGCCUGCCUACGGGGAAACCCGUAGGACGCU | |
| | UUAAUGUUAACAUGGUGCGAAGAGUCUACUGAGCUAGGUAGUAGUCCUCCGGCCCCUGAAUGCGGCU | |
| | AAUCCUAACCACGGAGCAGGUGCUCACACUCCAGUGAGUGGCCUGUCGUAACGCGCAAGUCUGUGGC | |
| | GGAACCGACUACUUUGGGUGUCCGUGUUUCCUUUUAUUUUUAUCAUGGCUGCUUAUGGUGACAAUCU | |
| | AAGAUUGUUAUCAUAUAGCUAUUGGAUUGGCCAUCCGGUGAACUCUAGAGAUAUUAUUUACUUGUUU | |
| | GUUGGUUUUAUCAAGUUAAAUAUCACCAGUUACACCACUCUCCUUUACAUUGUACUACUGAACAAUA | |
| | GAAAGAGAAA | |
| 270 | UUAAAACAGCCUCUGGGUUGCACCCACCCACAGGGCCCACUGGGCGCUAGCACACUGGUAUCACGGU | enterovirusA89 |
| | ACCUUUGUGCGCCUGUUUUAUACACCCUCCCCCCUUGUAACUUAGAAGAAACAAACAAACGAUCAAU | |
| | AGUAGGCUUAGCACUCCAGCUAAGUCUCGAUCAAGCACUUCUGUUACCCCGGACCGAGUAUCAAUAG | |
| | GCUGUACCCACGGCUGAAAGAGAAAACGUUCGUUACCCGGCUAUCUACUUCGGGAAACCCAGUACCA | |
| | UCAUGAAGAUUGCAAAGCGUUUCGCUCAGCACAACCCCAGUGUAGAUCAGGUCGAUGAGUCACUGCA | |
| | UUCCCCACAGGCGACUGUGGCAGUGGCUGCGUUGGCGGCCUGCCUAUGGGGAAACCCAUAGGACGCU | |
| | UUAAUAUUGACAUGGUGCGAAGAGUCUACUGAGCUAGGUAGUAGUCCUCCGGCCCCUGAAUGCGGCU | |
| | AAUCCCAACCACGGAGCAGGCGCUCACACUCCAGUGAGUAGCUUGUCGUAAUGCGAAAGUCUGUGGC | |
| | GGAACCGACUACUUUGGGUGUCCGUGUUUCCUUUUAUUUUUAUCAUGGCUGCUUAUGGUGACAAUCU | |
| | AAGAUUGUUAUCAUAUAGCUAUUGGAUUGGCCAUCCGGUGAAUUCUAGAGAUAUUAUUUACUUGUUU | |
| | GUUGGUUUUACCACAUUAAACAUUACCAAUUACAGAACUCUCCUCUACAUCCUGUUAUUGAAUAAUA | |
| | GAAAGAGAAA | |
| 271 | UUAAAACAGCUCUUAGGGUUGUUCCCACCCUAGAGGCCCACGUGGCGGCUAGCACUCUGGUAUCACG | enterovirusA90 |
| | GUACCUUUGUGCGCCUGUUUUAUACCCCCCUCCCCCAUGUAACUUAGAAGCUAUUAAAUAAAGUUCA | |
| | AUAGAAGGGGGUACAAACCAGUACCACCACGAACAAGCACUUCUGUUUCCCCGGUGAAGCUACAUAG | |
| | ACUGUUCCCACGGUUGAAAGUGGCAGAUCCGUUAUCCGCUUUGGUACUUCGAGAAACCUAGUAUCAU | |
| | CUUGGAAUCUUCGAUGCGUUGCGUUCAGCACUCAACCCCAGAGUGUAGCUUAGGUCGAUGAGUCUGG | |
| | ACGAUCCUCACUGGCGACAGUGGUCCAGGCUGCGUUGGCGGCCUACCUGUGGCGAAAGCUACAGGAC | |
| | GCUAGUUGUGAACAAGGUGUGAAGAGUCUAUUGAGCUAUCAAAGAGUCCUCCGGCCCCUGAAUGCGG | |
| | CUAUUCCCAACCACGGAGCAAGUGCUCACAAACCAGUGAGUGGCUUGUCGUAACGCGCAAGUCUGUG | |
| | GCGGAACCGACUACUUUGGGUGCCCGUGUUUCCUUUUAUUUUUAUCAUGGCUGCUUAUGGUGACAAU | |
| | CUAAGAUUGUUAUCAUAUAGCUAUUGGAUUGGCCAUCCGGUGACUAACAGAGAUAUCAUAUAUUCAU | |
| | UUGUUGGUUUUGUUAAACUAGACUCAACUACAUUCAAGUCUCUUCUCUUCAUCAUACAAUUGAAUAA | |
| | UAGAAAGAGAAA | |
| 272 | UUAAACAGCUCUAGGGUUGUUCCCACCCUAGAGGCCCACGUGGCGGCUAGCACUCUGGUAUCACGGU | enterovirusA91 |
| | ACCUUUGUGCGCCUGUUUUAUAUCCCUUUCCCCAUGUAACUUAGAAGUUAUUAAACAAAGUUCAAUA | |
| | GAAGGGGGUACAAACCAGUGCCACCACGAACAAGCACUUCUGUUUCCCCGGUGAAGCUGUAUAGACU | |
| | GUCUCCACGGUUGAAAAUGGCAGAUCCGUUACCCGCUUUGGUACUUCGAGAAACCUAGUAUCACCUU | |
| | GGAAUCUUCGAUGCGUUGCGCUCAGCACUCAACCCCAGAGUGUAGCUUAGGUCGAUGAGUCUGGACG | |
| | AUCCUCACUGGUGACAGUGGUCCAGGCUGCGUUGGCGGCCUACCUGUGGUGAAAACCACAGGACGCU | |
| | AGUUGUGAACAAGGUGUGAAGAGUCUAUUGAGCUACCAAAGAGUCCUCCGGCCCCUGAAUGCGGCUA | |
| | AUCCUAACCACGGAGCAAGUGCCUUCAAACCAGUAGGUAGCUUGUCGUAACGCGCAAGUCUGUGGCG | |
| | GAACCGACUACUUUGGGUGUCCGUGUUUCCUUUUAUUUUAUCAUGGCUGCUUAUGGUGACAAUCUAA | |
| | GAUUGUUAUCAUAUAGCUAUUGGAUUGGCCAUCCGGUGACUAACAGAGAUCUCAUAUACCUUUUUGU | |
| | UGGUUUUACUAAAUUAAAUAUUACCACGUUCAAGACACUUCUCCUUAUCAUACAACUAAAUAGUGAG | |
| | AAGAGAAA | |
| 273 | UUUAAAACAGCCUGUGGGUUGUACCCACCCACAGGGCCCACGCGGCGUUAGUACUCUGGUAUCACGG | enterovirusA92 |
| | UACCUUUGUAUGCCUGUUUUAUCUCCCUUCCCCCGUAACGUUAGAAGAAUGAACAAAAGACAAUUAG | |
| | CAAAUCCACCAACCCAGGUGGGUUUUCGUCAAGCACUUCUGUUUCCCCGGACCUAGUACCAAUAAGC | |
| | UGUGCCCACGGCUGAAGGGGAAAACGUUCGUUAUCCGGCUACCUACUUCGAGAAGCCUAGUACCACC | |
| | AAUGAUUGUCUCAAGUGUUACGCUCAGCACAACCCCAGUGUAGUUCAGGUCGAUGAGUCACCGAAUU | |
| | CCCCACGGGCGACCGUGUCGGUGGCUGCGUUGGCGGCCUGCCUGCGAGUUUCACUCGCAGGACGCUU | |
| | GAUUCAUGACAUGGUGCGAAGAGCCUAUUGAGCUAGGCAGUAGUCCUCCGGCCCCUGAAUGCGGCUA | |
| | AUCCUAACCACGGAGCCAUGCGUCACAAACCAGUGGCGUCAUGGUCGUAACGAGCAAUUCUGUGGCG | |
| | GAACCGACUACUUUGGGUGUCCGUGUUUCCUGUUUUAUUUACCUUGGCUGCUUAUGGUGACAAUCUA | |
| | GAGUUGUCAUCAUAUAGCGAUUGGAUUGGCCAUCCGGAUAUUACUUUGUGUGAUUUAUCUCUUGCUC | |
| | GUAAGCAAUUUUAAAUAGUUUAUAGCAUCAUU | |
| | UUAAAACAGCCUGUGGGUUGUACCCACCCACAGGGCCCACUGGGCGCUAGCACACUGAUUUCACGGA | enterovirusA120 |
| 274 | AUCCUUGUGCGCCUGUUUUACACCCCCUCCCCCCAACUCAGUAACUUAGAAGUUGUAAACACACGUG | |
| | GCCAACAGCGGGCGUAGCGCACCAGCUAUGUCUUGGUCAAGCACUUCUGUUUCCCCGGACAGAGUAU | |
| | CAAUAAGCUGCUUGCGCGGCUGAAGGAGAAAAUGUUCGUUACCCGGUCAACUACUUCGAGAAACCUA | |
| | GUAGCAUCAUGAAUGUUGCGAGGCGUUUCGCUCAGCACUUCCCCCGUGUAGAUCAGGUCGAUGAGUC | |
| | ACUGCACUCCCCACGGGCGACCGUGGCAGUGGCUGCGUUGGCGGCCUGCCCAUGGGGUGACCCAUGG | |
| | GACGCUUCAAUACGGACAUGGUGUGAAGAGUCUAUUGAGCUAGUUGAUAAUCCUCCGGCCCCUGAAU | |
| | GCGGCUAAUCCUAACUGCGGAGCACAUACCCUCAACCCAGGGGGCGGUGUGUCGUAACGGGCAACUC | |
| | UGCAGCGGAACCGACUACUUUGGGUGUCCGUGUUUCCUUUUAUACUUAUACUGGCUGCUUAUGGUGA | |
| | CAAUUGAAGAAUUGUUACCAUAUAGCUAUUGGAUUGGCCAUCCGGUGACUAACAGAGCUAUUAUAUA | |
| | UUUAUUUGUUGGAUUUGUUCCAUUAACAUACACAUCUUUCAGCACUUUACACCUCAUUUUACAAUAC | |
| | AAUACAAGAAA | |
| 275 | UUUAAAACAGCUCUGGGGUUGUUCCCACCCUAGAGGCCCACGUGGCGGCUAGCACUCUGGUAUUACG | enterovirusA121 |
| | GUACCUUUGUGCGCCUGUUUUAUAUUCCCUUCCCCACGCAACUUAGAAGUUAUUAAACAAAGUUCAA | |
| | UAGAAGGGGGUACAAACCAGUACCACCACGAACAAGCACUUCUGUUUCCCCGGUGAAGCUACAUAGA | |
| | CUGUUCCCACGGUUGAAAGUGGCAGAUCCGUUAUCCGCUUUGGUACUUCGAGAAACCUAGUAUCACC | |
| | UUGGAAUCUUCGAUGCGUUGCGCUCAGCACUCAAUCCCAGAGUGUAGCUUAGGCUGAUGAGUCUGGA | |
| | CGAUCCUCACUGGCGACAGUGGUCCAGGCUGCGUUGGCGGCCUACCUGUGGUGAAAGCCACAGGACG | |
| | CUAGUUGUGAACAAGGUGUGAAGAGUCUAUUGAGCUACCAAAGAGUCCUCCGGCCCCUGAAUGCGGC | |
| | UAAUCCCAACCACGGAGCAAGCGCUCACAAACCAGUGAGUAGCUUGUCGUAACGCGCAAGUCUGUGG | |
| | CGGAACCGACUACUUUGGGUGUCCGUGUUUCCUUUUAUUUUUAUCAUGGCUGCUUAUGGUGACAAUC | |
| | UAAGAUUGUUAUCAUAUAGCUUUUGGAUUGGCCAUUCGGUGACUAAUAGAGACCUUGUGUAUCUAUU | |
| | UGUUGGUUUUACCAAACUAAAUACUAUUACACUCAAAACACUCCUUCUUAUUAUACAACUUAAUAGU | |
| | AGAAAGAGAAA | |
| 276 | UUAAAACAGCCUGUGGGUUGUACCCACCCGCAGGGCCCACUGGGCGCUAGUACACUGAUAUCACGGU | enterovirusA122; |
| | ACCUUUGUACGCCUGUUUUAUCUCCCUACCCCCAUCGUAACUUAGAAGCUUAACAAUACAGAUCAAU | simianvirus19 |
| | AGCCUCUGCACCAAACCAGGUGUGGAAUGAUCAAGCAUUUCUGUUACCCCGGACCUAGUACCAAUAG | |
| | GCUGUGUCAAACGGCUGAAGGGGAAAAAGUUCGUUACCCGGCCACCUACUUCGAGAAGCCUAGUAGC | |
| | AUCACUGAAGCUGUGAAGCGUUACGAUCAGCACAACCCCAGUGUAGUUCAGGUCGAUGAGUCACUGC | |
| | AUUCCCCACAGGCGACUGUAGCAGUGGCUGCGUUGGCGGCCUGCCCUUGGUCUCUGACCAAGGGACG | |
| | CUUUGGAGUGGAUAUGGUGCGAAGAGCCUAUUGAGCUAGGUGGUAGUCCUCCGGCCCCUGAAUGCGG | |
| | CUAAUCCUAACCCCGGAGCUGGUGCACACAAACCAGUGUGUGGCCAGUCGUAAUGAGCAAUUCUGGG | |
| | AUGGAACCGACUACUUUGGGUGUCCGUGUUUCCUGUUUCACUUACCUUGGCUGCUUAUGGUGACAAU | |
| | UGUAUAGUUGUCAUCAUAUAGCUAUUGGAUUGGCCAUCCGGAUAUUAUUCUUGUGUUUAUUUGUGUA | |
| | AUUUGAUAUAAUUUAGAACCCUGUAUAAGA | |
| 277 | UUAAAACAGCCUGUGGGUUGUUCCCACCCACAGGGCCCACUGGGCGCUAGUACUCUGGUAUUACGGU | enterovirusA123; |
| | ACCUUUGUACGCCUGUUUUACAACCCUUCCCCCAUGUAACUUAGAAGCAACAAAUAAACGACCAAUA | simianvirus43 |
| | GCACACUCACCAAUCCAGAUGAGUGAUGGUCAAGCACUUCUGUUACCCCGGGCUUAGUACCAAUAGA | |
| | CUGCCAUCGCGGUUGAAGGGGAAAACGCUCGUUAUCCGGCUCUCUACUGCGAGAAGCCUAGUAACGC | |
| | CUUAGAAAUGCUCAAGCGUUGCGCUCAGCACAACCCCAGUGUAGCUCAGGUCGAUGAGUCACUGCAU | |
| | UCCCCACGGGCGACCGUGGCAGUGGCUGCGUUUGCGGCCUGCCUACAGCUCUACGCUGUAGGACGCU | |
| | UGUGUAUGGACAUGGUGUGAAGAGCCCAUUGCGCUAGGGAGUAGUCCUCCGGCCCCUGAAUGCGGCU | |
| | AAUCCUAACCCCGGAGCUGGUGCUUGUAAACCAACAAGUGGCCAGUCGUAAUGGGCAACUCUGGGAU | |
| | GGAACCGACUACUUUGGGUGUCCGUGUUUCCUGUUUUAUUUACCUUGGCUGCUUAUGGUGACAAUCU | |
| | AGUGUUGUUGCCAUAUAGCGAUUGGAUUGGCCAUCCGGAUAUUGUUUUAGACACUUGUGUUUGUGAG | |
| | UAGAUCUCUUCUGACAUAGGAUAUAACA | |
| 278 | UUAAAACAGCCUGUGGGUUGUACCCACCCACAGGGCCCACUGGGCGCUAGCACUCUGGUAUUACGGU | enterovirusA |
| | ACCUUUGUGCGCCUGUUUUAUUUCCCUUCCCCAUUGUAACUUUAGAAGAAACAAAACUGGAUCAAUA | 124; |
| | GCCCAUCACCCAAACCAGGGUGGUGACGAUCAAGCACUUCUGUUUCCCCGGAUCUAGUAUCAAUAGA | simianvirus46 |
| | CUGUUAGCACGGUUGAAGGAGAACGAUUCGUUAUCCGGCUCACUACUUCGAGAAGCCUAGUACCACU | |
| | AUGAAAGUGAAAUAGCGUUUCGCUCAGCACAACCCCAGUGUAGAUCAGGCCGAUGAGUCACUGCAUU | |
| | CCCCACGGGCGACCGUGGCAGUGGCUGCGUUGGCGGCCUGCCUGUGGGUCCCCCCACAGGACGCUAA | |
| | UUCAUGGACAUGGUGCGAAGAGCCUAUUGAGCUAGUGAGUAGUCCUCCGGCCCCUGAAUGCGGCUAA | |
| | UCCUAACCACGGAGCAUCCGCUGGCAGUCCAGCCAGUAGGGUGUCGUAACGGGUAACUCUGUGGCGG | |
| | AACCGACUACUUUGGGUGUCCGUGUUUCCUGUUUUACUUACUUUGGCUGCUUAUGGUGACAAUUUAC | |
| | GGAUUGUUGCCAUAUAGCUAUUGGAUUGGCCAUCCGGAUUAUAAACACACAAUACUGUUAUUUUUUC | |
| | CUUUUUGGGAUUUCUUCCCUUGAGUUUUACUAAUUAUUUUAUCUUGUUAAUAGUUUUAAAGUUGAAU | |
| | ACAUU | |
| 279 | UUAAAACAGCCUGUGGGUUGUUCCCACUCACAGGGCCCAAGUGGCGUAGAACUCUGGUAUCACGGUA | enterovirusA125; |
| | CCUUUGUUCGCCUAUUUUAAAACCCCCCCCCCCGUAACUUAGAAGAAUGAACAAAACAAACUCAGUA | baboon- |
| | GUAGGUAAUGCAUCCAGCAUUAUCACGAGUUAAGCACUUCUGUUUCCCCGGUGAUGGAGAUAGGCUG | enterovirusA13 |
| | UACCCACGGCUGAAACUCUAUGAUCCGUUAUCCGGUAUGGUACUGCGAGAAGUCUAGUACCGUCAUG | |
| | AAAGUUGCUAAGCGUUACGCUCCCCAGUAACCCCAACUGGUAGUUCUGGCUGAUGAGUCUGGGCACU | |
| | CCCCACAGGCGACUGUGGCCCAGGCUGCGUUGGCGGCCAGCUCAUCCUCAUUGAGGGUGAGACGCUU | |
| | AAAUACAGACAUGGUGAGAAGAACCUAUUGCGCUACCAUAUAGUCCUCCGGCCCCUGAAUGCGGCUA | |
| | AUCCCAACCUCGGAGCAUUCAACUGCAAACCAGCAGUGAGAGUGUCGUAACGCGAAAGUCCGAGGCG | |
| | GAACCGACUACUUUGGGUGUCCGUGUUUCCUGUUUUACCUUUUGAUGGCUGUUUAUGGUGACAAUUU | |
| | AGAGUAUUCAGAUUGUCAUCAUAAACUACUAGGUCUUGCCAACCGGUCUAUAAUAUAUUAUUUUGUU | |
| | GGAUUUGUUCCACUCACGCCAAUUAUUUACAAAAUUUUGCUUAGAUUCCUACUCGCAAACUUAAGAU | |
| | AUCAAAG | |
| 280 | UUAAAACAGCCUGUGGGUUGAUCCCACCCACAGGGCCCAUUGGGCGCUAGCACUCUGGUAUCACGGU | coxsackievirB3 |
| | ACCUUUGUGCGCCUGUUUUAUACCCCCUCCCCCAACUGUAACUUAGAAGUAACACACACCGAUCAAC | |
| | AGUCAGCGUGGCACACCAGCCACGUUUUGAUCAAGCACUUCUGUUACCCCGGACUGAGUAUCAAUAG | |
| | ACUGCUCACGCGGUUGAAGGAGAAAGCGUUCGUUAUCCGGCCAACUACUUCGAAAAACCUAGUAACA | |
| | CCGUGGAAGUUGCAGAGUGUUUCGCUCAGCACUACCCCAGUGUAGAUCAGGUCGAUGAGUCACCGCA | |
| | UUCCCCACGGGCGACCGUGGCGGUGGCUGCGUUGGCGGCCUGCCCAUGGGGAAACCCAUGGGACGCU | |
| | CUAAUACAGACAUGGUGCGAAGAGUCUAUUGAGCUAGUUGGUAGUCCUCCGGCCCCUGAAUGCGGCU | |
| | AAUCCUAACUGCGGAGCACACACCCUCAAGCCAGAGGGCAGUGUGUCGUAACGGGCAACUCUGCAGC | |
| | GGAACCGACUACUUUGGGUGUCCGUGUUUCAUUUUAUUCCUAUACUGGCUGCUUAUGGUGACAAUUG | |
| | AGAGAUUGUUACCAUAUAGCUAUUGGAUUGGCCAUCCGGUGACCAAUAGAGCUAUUAUAUAUCUCUU | |
| | UGUUGGGUUUAUACCACUUAGCUUGAAAGAGGUUAAAACAUUACAAUUCAUUGUUAAGUUGAAUACA | |
| | GCAAA | |
| 281 | UUAAAACAGCCUGUGGGUUGUUCCCACCCACAGGCCCAUUGGGCGCUAGCACUCUGGUAUCACGGUA | coxsackievirB1 |
| | CCUUUGUGCGCCUGUUUUACAUCCCCUCCCCAAAUUGUAAUUUAGAAGUUUCACACACCGAUCAUUA | |
| | GCAAGCGUGGCACACCAGCCAUGUUUUGAUCAAGCACUUCUGUUACCCCGGACUGAGUAUCAAUAGA | |
| | CCGCUAACGCGGUUGAAGGAGAAAACGUUCGUUACCCGGCCAACUACUUCGAAAAACCUAGUAACAC | |
| | CAUGGAAGUUGCGGAGUGUUUCGCUCAGCACUACCCCAGUGUAGAUCAGGUCGAUGAGUCACCGCGU | |
| | UCCCCACGGGCGACCGUGGCGGUGGCUGCGUUGGCGGCCUGCCUACGGGGAAACCCGUAGGACGCUC | |
| | UAAUACAGACAUGGUGCGAAGAGUCUAUUGAGCUAGUUGGUAAUCCUCCGGCCCCUGAAUGCGGCUA | |
| | AUCCUAACUGCGGAGCACAUACCCUCAAACCAGGGGGCAGUGUGUCGUAACGGGCAACUCUGCAGCG | |
| | GAACCGACUACUUUGGGUGUCCGUGUUUCAUUUUAUUCCUAUACUGGCUGCUUAUGGUGACAAUUGA | |
| | CAGGUUGUUACCAUAUAGUUAUUGGAUUGGCCAUCCGGUGACUAACAGAGCAAUUAUAUAUCUCUUU | |
| | GUUGGGUUUAUACCACUUAGCUUGAAAGAGGUUAAAACACUACAUCUCAUCAUUAAACUAAAUACAA | |
| | CAAA | |
| 282 | UUAAAACAGCCUGUGGGUUGAUCCCACCCACAGGGCCCACUGGGCGCUAGCACACUGGUAUUACGGU | coxsackievirB2 |
| | ACCUUUGUGCGCCUGUUUUAUCUACCCUUGCCCAAUGCAACAUAGAAGCUUAGUCCAAAAGGUCAAU | |
| | AGGCAGCCCAGUGCACCAACUGGGUCCAGACCAAGCACUUCUGUUACCCCGGACCAAGUAUCAGUAG | |
| | GCUGCUCACGCGGUGAAGGAGAAAACGUUCGUUACCCGGCCAACUACUUCGAGAAACCUAGUAGCAC | |
| | CAUGAAGGUUGCGCAGUGUUUCGCUCCGCACAGCCCCAGUGUAGAUCAGGUCGAUGAGCCACCGCAU | |
| | UCCCCACGGGGUGACCGUGGCGGGUGGCUGCGUUGGCGGCCUGCCCAUGGGGUAACCCAUGGGACGC | |
| | UUCAAUACUGACAUGGUGUGAAGAGUCUAUUGAGCUAGUUGGUAGUCCUCCGGCCCCUGAAUGCGGC | |
| | UAAUCCUAACUGCGGAGCGUGCGCUUGCAACCCAGUGAGUAGCACGUCGUAAUGGGUAACUCUGCAG | |
| | CGGAACCGACUACUUUGGGUGUCCGUGUUUCCUUUAUUCCUUGUCUGGCUGCUUAUGGUGACAAUUG | |
| | AGAGAUUGUUGCCAUAUAGCUAUUGGAUUGGCCAUCCGGUAUCUAACAGGGCGAUCAUUUACCUGUU | |
| | UGUUGGUUUCAUACCCUUGAAUCUUGAGGUUUAUAAUACCCUCACUCUUAUAAUAAAACUCAAUACA | |
| | UCAAG | |
| 283 | UUAAAACAGCCUGUGGGUUGUACCCACCCACAGGGCCCAAUGGGCGCUAGCACACUGGUAUUCCGGU | coxsackievirB4 |
| | ACCUUUGUGCGCCUGUUUUAUAACCCCCCCCCAGUUCGCAACUUAGAAGCAAAGAAACAAUGGUCAA | |
| | UUACUGACGCAGCAACCCAGCUGUGUUUUGGCCAAGUACUUCUGUGUCCCCGGACUGAGUAUCAAUA | |
| | AGCUGCUUGCGCGGCUGAAGGAGAAACCGUUCGUUACCCGGCCAACUACUUCGAGAAGCCUAGUAAC | |
| | GCCAUGAACGUUGAGGAGUGUUUCGCUCAGCACUUCCCCCGUGUAGUUCAGGUCGAUGAGUCACCGC | |
| | GUUCCCCACGGGUGACCGUGGCGGUGGCUGCGUUGGCGGCCUGCCUGUGGGGCAACCCGCAGGACGC | |
| | UCUGAUACAGACAUGGUGUGAAGAGCCUAUUGAGCUAGUUGGUAGUCCUCCGGCCCCUGAAUGCGGC | |
| | UAAUCCUAACUGCGGAGCACACGUUCGCAAGCCAGCGAGUGGUGUGUCGUAACGGGCAACUCUGCAG | |
| | CGGAACCGAGUACUUUGGGUGUCCGUGUUUCCUUUUAUUCUUACCUUGGCUGCUUAUGGUGACAAUU | |
| | GAAAGAUUGUUACCAUAUAGCUAUUGGAUUGGCCAUCCAGUGUCAAAUAGAGCAAUCAUAUAUCUGU | |
| | UUGUUGGUUUCGUUCCCUUGGACUACAGAAAUCUUAAAACUCUUUAUUUCAUAUUGAGACUCAAUAC | |
| | GAUAAA | |
| 284 | UUAAAACAGCCUGUGGGUUGUUCCCACCCGCAGGGCCCACUGGGCGCCAGCACUCUGGUAUCACGGU | coxsackievirB5 |
| | ACCUUUGUGCGCCUGUUUUAAAACCCUCUCCCCAAUUUGAAACUUAGAAGCAAUACACCUCGAUCAA | |
| | UAGUAGGCAUGACACGCCAGCCAUGUCUUGAUCAAGCACUUCUGUUUCCCCGGACUGAGUAUCAAUA | |
| | AACUGCUUGCGCGGUCGAAGGAGAAAACGUCCGUUACCCGACUAACUACUUCGAGAAACCCAGUAAC | |
| | ACCAUGGAAAUUGCGGAGUGUUUCACUCAGCACAUUCCCAGUGUAGAUCAGGUCGAUGAGUCACCGC | |
| | AUUCCCCACGGGUGACCGUGGCGGUGGCUGCGCUGGCGGCCUGCCCAUGGGGCAACCCAUGGGACGC | |
| | UUCAAUAUGGACAUGGUGUGAAGAGUCUAUUGAGCUAGUUAGUAGUCCUCCGGCCCCUGAAUGCGGC | |
| | UAAUCCUAACUGCGGAGCACGUGCCUCCAUUCCAGGGGGUGGCGUGUCGUAACGGGCAACUCUGCAG | |
| | CGGAACCGACUACUUUGGGUGUCCGUGUUUCUUUUAAUUUUAUACUGGCUGCUUAUGGUGACAAUUG | |
| | AAAGAUUGUUGCCAUAUAGCUAUUGGAUUGGCCAUCCGGUAUCCAACAGAGCAAUUGUGUACCUUUU | |
| | UGUUGGAUUUGUACCACUUACCAGAACAAGUUUUCAUACACUGUGUUACAUUAUUAGACUAAACACA | |
| | GAAAA | |
| 285 | UUAAAACAGCCUGUGGGUUGUACCCACCCACAGGGCCCACUGGGCGCUAGCACUCUGGUAUUGCGGU | coxsackievirB6 |
| | ACCUUUGUGCGCCUGUUUUACCCACCCUCCCCCAACGUAACUUAGAAGCCUGACAUAUACGGUCACA | |
| | AGCCAGCCCAGUACGCCAACUGGGUACCGACCAAGCACUUCUGUUACCCCGGACUGAGUAUCAAUAA | |
| | GCUGCCAAAGCGGCUGAAGGAGAAAACGUUCGUUACCCGGCCAAUUACUUCGAGAAACCCAGUAACA | |
| | CCAUGAAAGUUGCGUAGCGUUUCAUUCAGCACAAUCCCAGUGUAGAUCAGGUCGAUGAGUCACCGCA | |
| | UUCCCCACAGGCGACUGUGGCGGUGGCUGCGUUGGCGGCCUGCCCAUGGGGCAAACCCAUGGGACGC | |
| | UUCAAUACUGACAUGGUGCGAAGAGUCUAUUGAGCUAAUUGGUAGUCCUCCGGCCCCUGAAUGCGGC | |
| | UAAUCCUAACUGCGGAGCAGGCACUCGCAGACCAGCGAGCAGCUUGUCGUAAUGGGCAACUCCGCAG | |
| | CGGAACCGACUACUUUGGGUGUCCGUGUUUCCUAUUUCCUUUACACUGGCUGCUUAUGGUGACAAUU | |
| | GAGAAAUUGUUACCAUAUAGCUAUUGGAGUGGCCAUCCGGUGACUAACAGAGCAAUAAUAUAUUUGU | |
| | UUGUUGGCUUUGUGCCACUUAAUUUGAAGGUUGUUAAGACGCUCAAUCUGAUACUACUUCUAAAUAA | |
| | AGCAAA | |
| 286 | UUUAAAACAGCCUGUGGGUUGUUCCCACCCACAGGCGCCACCGGGCGUUAGCACACUGGUAUCACGG | coxsackievirA9 |
| | UACCCUUGUGCGCCUGUUUUAUAACCCCACCCCGAGUAAACCUUAGAAGCAAUGCACCUCUGGUCAA | |
| | UAGUAGGUGUGACACACCAGUCACAUCGUGACCAAGCACUUCUGUCUCCCCGGACUGAGUAUCAAUA | |
| | GGCUGCUCGCGCGGCUGAAGGAGAAAGCGUUCGUUACCCGGCCAGCUACUUCGAGAAGCCUAGUAAC | |
| | ACCAUGAAGGUUGCAGAGUGUUUCGCUCAGCACUUCCCCUGUGUAGAUCAGGCCGAUGAGUCACCGC | |
| | GUUCCUCACGGGCGACCGUGGCGGUCGCUGCGCUGGUGGCCUGCCUAUGGGGCAACCCAUAGGACGC | |
| | UCUAAUGCUGACAUGGUGCGAAGAGUCUAUUGAGCUAGCUGGUAGUCCUCCGGCCCCUGAAUGCGGC | |
| | UAAUCCCAACUGCGGAGCACGCACCCUCAAACCAGGGGGCAGCGUGUCGUAACGGGCAACUCUGCAG | |
| | CGGAACCGACUACUUUGGGUGUCCGUGUUUCUUUUUAUUCCUAUAUUGGCUGCUUAUGGUGACAAUU | |
| | GAGAGAUUGUUACCAUAUAGCUAUUGGAUUGGCCAUCCGGUGAGCAACAGAGCUAUUGUGUAUCUGU | |
| | UUACUGGUUUCAUACCCCUUAAUUACAAAGAAGUCAAAACCCUUCACUUGAUCUUGUUAUUCAAUAC | |
| | AACAAA | |
| 287 | UUAAAACAGCCUGUGGGUUGUUCCCACCCACAGGGCCCACUGGGCGUUAGCACACUGGUAUCACGGU | echovirus1 |
| | ACCUUUGUGCGCCUGUUUUAUACUCCCCCCCCUAAGGAAACUUUAGAAGCAAAGCAAUUGUGAUCAA | |
| | UAGUGGGUAUGGCACACCAGUCAUAUCUUGAUCAAGCACUUCUGUUCCCCCGGACUUAGUACCAAUA | |
| | GACUGCUCAAGCGGUUGAAGGGGAAAACGUUCGUUAUCCGGCCAACUACUUCGAGAAACCUAGUAGC | |
| | ACCAUGAAAGUUGCGGAGUGUUUCGCUCAGCACUUCCCCCGUGUAGAUCAGGCUGAUGAGUCACCGU | |
| | AUUCCCCACGGGCGACCGUGACGGUGGCUGCGUUGGCGGCCUGCCCAUGGGGUAACCCAUGGGACGC | |
| | UCUAAAACAGACACGGUGCGAAGAGUCUAUUGAGCUAGUUGGUAGUCCUCCGGCCCCUGAAUGCGGC | |
| | CAAUCCUAACUGCGGAGCACAUACUCCCAAUCCAGGGAGCAGUGUGUCGUAAUGGGUAACUCUGCAG | |
| | CGGAACCGACUACUUUGGGUGUCCGUGUUUCCUUUUAUUCUCACAUUGACUGCUUAUGGUGACAAUU | |
| | GAAAGAUUGUUACCAUAUAGCUAUUGGAUUGGUCAUCCGGUGAGCAAUAGAGCUAUUGUUUAUCAAU | |
| | UUGUUGGAUUUGUACCACUCAACUUUUCUGUUUUGAGAACACUCAACUACAUCUUACUGCUAAACAC | |
| | AUCAAA | |
| 288 | UUAAAACAGCCUGUGGGUUGAUCCCACCCACAGGGCCCAUUGGGCGCUAGCACUCUGGUAUUACGGU | echovirus2 |
| | ACCCUUGUGCGCCUGUUUUACACGCCCCUCCCCAAGCAACUUAGAAGUCUGUCACAAAUGGUCAAUA | |
| | GACAGCUCAACACGCCAGUUGAGUCUAGACCAAGCACUUCUGUCGCCCCGGACCGAGUAUCAAUAGA | |
| | CUGCUAGCGCGGUUGAAAGAGAAAACGUUCGUUACCCGGCCAACUACUUCGGGAAACCUAGUACCAC | |
| | CAUGAAAGUUGCGCAGUGUUUCGCUCCGCACAACCCCAGUGUAGAUCAGGUCGAUGAGCCACCGCAU | |
| | UCCCCACAGGCGACUGUGGCGGUGGCUGCGUUGGCGGCCUGCCCAUGGUACAACCCAUGGGACGCUU | |
| | CAAUACUGACAUGGUGCGAAGAGUCUACUGAGCUAGUUGGUAGUCCUCCGGCCCCUGAAUGCGGCUA | |
| | AUCCUAACUGCGGAGCAGGUACCCACGAGCCAGUGGGCAGCCUGUCGUAAUGGGCAACUCUGCAGCG | |
| | GAACCGACUACUUUGGGUGUCCGUGUUUCCAUUUACUCUUAUACUGGCUGCUUAUGGUGACAAUCAC | |
| | GGAAUUGUUACCAUAUAGCUAUUGGAUUGGCCAUCCAGUGACUAACAGAGCGAUUAUAUUCCUUUUU | |
| | GUUGGUUUUGUACCACUCACCGUCACGAGCUUUACAACGCUAAACUACAUUUUGUCAUUGAACACGG | |
| | CAAG | |
| 289 | UUAAAACAGCCUGUGGGUUGUACCCACCCACAGGGCCCACUGGGCGCCAGCACUCUGGUAUUACGGU | echovirus3 |
| | ACCCUUGUGCGCCUGUUUUAUAUCCCUUCCCCAAGGUAAACGUUAGAAGCAAUGCACUCCCGAUCAA | |
| | UAGAAGGUGCGACGCGCCAGUCGUAUCUCGAUCAAGCACUUCUGUUUCCCCGGACCGAGUAUCAAUA | |
| | GACUGCUAGCGCGGUUGAAGGAGAAAACGUUCGUUACCCGACUAACUACUUCGAGAAGCCCAGUAGC | |
| | GCCAUGAAAGUUGCAGAGUGUUUCGCUCAGCACUUCCCCCGUGUAGAUCAGGCCGAUGAGUCACCGC | |
| | AAUCCCCACGGGCGACCGUGGCGGUGGCUGCGUUGGCGGCCUGCCUAUGGGGAAACCCAUAGGACGC | |
| | UCAAAUACAGACAUGGUGCGAAGAGUCUAUUGAGCUAGUUAGUACUCCUCCGGCCCCUGAAUGCGGC | |
| | UAAUCCUAACUGCGGAGCAAGUACUCACAAUCCAGUGGGCAGCUUGUCGUAACGGGUAACUCUGCAG | |
| | CGGAACCGACUACUUUGGGUGUCCGUGUUUCCUUUUAUUCCUACAUUGGCUGCUUAUGGUGACAAUC | |
| | AAAGAGUUGUUACCAUAUAGCUAUUGGAUUGGCCAUCCGGUGACUAAUAAAGCAAUUAUAUUUCUUU | |
| | UUGUUGGGUUUGUACCCCUUAAUACAACCAGCUUCACCACAUUGUGUUACAUCAUUAGACUGAACAA | |
| | GGCAAA | |
| 290 | UUAAAACAGCCUGUGGGUUGCACCCACCCACAGGGCCCAUUGGGCGCUAGCACCCUGGUAUUACGGU | echovirus4 |
| | ACCUUUGUGCGCCUGUUUUAUACCCCCAUCCCCAAUCGAAACUUAGAAGCAUUACACACUGAUCAAU | |
| | AGGAGGCGCGGCACGCCAGCCAUGCCAAGAUCAAGCACUUCUGUCUCCCCGGACCGAGUAUCAAUAG | |
| | ACUGCUUGCGCGGUUGAAGGAGAAAACGUUCGUUACCCGGCCAACUACUUCGAGAAACCUAGUACCA | |
| | CCAUGAAAGUUGCGGAGUGUUUCGCUCAGCACUACCCCAGUGUAGAUCAGGCCGAUGAGUCACCGCG | |
| | UUCCCCACGGGUGACCGUGGCGGUGGCUGCGCUGGCGGCCUGCCUAUGGGGCAACCCAUAGGACGCU | |
| | CUAAUACAGACAUGGUGUGAAGAGUCUAUUGAGCUAGUUGGUGAUCCUCCGGCCCCUGAAUGCGGCU | |
| | AAUCCUAACUGCGGAGCACACGCUCACAAGCCAGUGAGUGGUGUGUCGUAAUGGGUAACUCCGCAGC | |
| | GGAACCGACUACUUUGGGUGUCCGUGUUUCCUUUUAACUUCAUUUUGGCUGCUUAUGGUGACAAUUA | |
| | AGAAAUUGUUACCAUAUAGCUAUUGGAUUGGCCAUCCGGUGACUAGUAGAGCUAUUAUAUACUUGUU | |
| | UGUUGGCUUUGUACCACUAAACUAUAAAGUCCUUAGAACUCUUGAUUUUAUACUAAUUUUGAAUAAG | |
| | GCAAA | |
| 291 | UUAAAACAGCCUGUGGGUUGCACCCACCCACAGGGCCCACUGGGCGCUGGUACACUGGUAUUAAGGU | echovirus5 |
| | ACCCUUGUACGCCUGUUUUAUAUCCCUCCCCCGAUUUGUAACUUAGAAUUAAAGCAAACCCGAUCAA | |
| | UAGUAGGUGUGGCAAACCAGCCACAUCUUGAUCAAGCACUUCUGUUUCCCCGGACCGAGUAUCAAUA | |
| | GACUGCUGACGCGGUUGAAGGAGAAAACGUUCGUUACCCGGCCAACUACUUCGGGAAACCUAGUACC | |
| | ACCAUGAAGGUUGCGCAGUGUUUCGCUCCGCACAACCCCAGUGUAGAUCAGGUCGAUGAGCCACCGC | |
| | AUUCCCCACAGGCGACUGUGGCGGUGGCUGCGUUGGCGGCCUGCCCAUGGGGUAACCCAUGGGACGC | |
| | UUCAAUACUGACAUGGUGCGAAGAGUCCACUGAGCUAGUUGGUAUUCCUCCGGCCCCUGAAUGCGGC | |
| | UAAUCCUAACUGCGGAGCAGGCACCCACGAGCCAGUGGGCAGCCUGUCGUAAUGGGUAACUCUGCAG | |
| | CGGAACCGACUACUUUGGGUGUCCGUGUUUCCAUUUUCUUUUUAUUGGCUGCUUAUGGUGACAAUCA | |
| | CGGAAUUGUUACCAUAUAGCUAUUGGAUUGGCCAUCCAGUGACUAACAGAGCAGUAAUAUUCCUUUU | |
| | UGUUGGUUUUGUACCACUCACCGUCACGAGCCUCAGAACACUCGUAUUCAUCUUAACCCUUAACAAG | |
| | AUAAA | |
| 292 | UUAAAACAGCCUGUGGGUUGCCCCCACCCGCAGGGCCCAUUGGGCGCUAGCACACUGGUAUCACGGU | echovirus6 |
| | ACCUUUGUGCGCCUGUUUUAUCCUCCCUCCCCAAGUGCAACGUAGAAGCAAGACACAAAAGGUCAAC | |
| | AGAUAGCCCAGCACACCAGUUGGGUUUCGACCAAGCACUUCUGUCUCCCCGGACCGAGUAUCAAUAA | |
| | GCUGCUCACGCGGCUGAAGGAGAAAGUGUUCGUUACCCGGCUAGUUACUUCGAGAAACCUAGUACCA | |
| | CCAUGAAGGUUGCGCAGCGUUUCGCUCCGCACAACCCCAGUGUAGAUCAGGUCGAUGAGUCACCGCG | |
| | UUCCCCACGGGCGACCGUGGCGGUGGCUGCGUUGGCGGCCUGCCCAUGGGGCAACCCAUGGGACGCU | |
| | UCAAUACUGACAUGGUGCGAAGAGUCUAUUGAGCUAACUAGUAGUCCUCCGGCCCCUGAAUGCGGAU | |
| | AAUCUUAACUGCGGAGCAGGUGCUCACAAUCCAGUGGGUGGCCUGUCGUAACGGGCAACUCUGCAGC | |
| | GGAACCGACUACUUUGGGUGUCCGUGUUUCCUUUUAUCUCUUUGUUGGCUGCUUAUGGUGACAAUUG | |
| | AACGAUUGUUGCCAUAUAGCUAUUGGAUUGGCCAUCCGGUGAGCAGCAGAUCCAUUAUUUACCAGUU | |
| | UGUUGGAUUUGUACCACUUGAUCACAUAGUCUUUAGAACACUUGUCUUUAUUCUAAAUCUUAACAAG | |
| | GCAAG | |
| 293 | UUAAAACAGCCUGUGGGUUGUACCCACCCACAGGGCCCAUUGGGCGUCAGCACCCUGGUAUCACGGU | echovirus7 |
| | ACCUUUGUGCGCCUGUUUUAUAUCCCUUCCCCCAAUUGUAACUUAGAAGAAACACACACCGAUCAAC | |
| | AGCAAGCGUGGCACACCAGCCAUGUUUUGGUCAAGCACUUCUGUUACCCCGGACUGAGUAUCAAUAG | |
| | ACUGCUCACGCGGUUGAAGGAGAAAGCGUCCGUUAUCCGGCCAGCUACUUCGAGAAACCUAGUAACA | |
| | CCAUGGAAGUUGCGGAGUGUUUCGCUCAGCACUACCCCAGUGUAGAUCAGGUCGAUGAGUCACCGCU | |
| | UUCCCCACGGGCGACCGUGGCGGUGGCUGCGUUGGCGGCCUGCCUAUGGGGGAACCCAUAGGACGCU | |
| | CUAAUACAGACAUGGUGCGAAGAGUCUAUUGAGCUAGCUGGUAUUCCUCCGGCCCCUGAAUGCGGCU | |
| | AAUCCUAACUGUGGAGCACAUGCCCCUAAUCCAAGGGGUAGUGUGUCGUAAUGAGCAAUUCCGCAGC | |
| | GGAACCGACUACUUUGGGUGUCCGUGUUUCCUCUUAUUCUUGUACUGGCUGCUUAUGGUGACAAUUG | |
| | AGAGAUUGUUACCAUAUAGCUAUUGGAUUGGCCAUCCGGUGACUAAUAGAGCUAUUGUGUAUCUCUU | |
| | UGUUGGAUUUGUACCACUUAAUUUGAAAGAAAUCAGGACACUACGCUACAUUUUACUAUUGAACACC | |
| | GCAAA | |
| 294 | UUAAAACAGCCUGUGGGUUGUACCCACCCACAGGGCCCACUGGGCGCCAGCACUCUGGUAUCGCGGU | echovirus9 |
| | ACCUUUGUGCGCCUGUUUUGUUUCCCUUUACCCCGAAUGGAACUUAGAAGUAAUGCACACUGAUCAA | |
| | UAGCAGGCGUGGCACCAGCCACGUGAUGAUCAAGCACUACUGUUUCCCCGGACCGAGUAUCAAUAGA | |
| | CUGCUUGCGCGGUUGAAGGAGAAAACGUUCGUUAUCCGGCUAACUACUUCGAGAAACCCAGUAACAC | |
| | CAUGAAGGUUGCAGGCGAUUUCGCUCAGCACAUCCCCAGUGUAGAUCAGGUCGAUGAGUCACCGCGU | |
| | UCCCACGGGCGACCGUGGCGGUGGCUGCGUUGGCGGCCUGCCUAUGGGGUAACCCAUAGGACGCUCU | |
| | AAUACGGACAUGGUGCGAAGAGUCUAUUGAGCUAGUUAGUGUUCUCCGGCCCCUGAAUGCGGCUAAU | |
| | CCUAACUGUGGAGCACAUGCCCCUAAUCCAAGGGGUAGUGUGUCGUAACGGGCAACUCCGCAGCGGA | |
| | ACCGACUACUUUGGGUGUCCGUGUUUCCUUUUAUUCUUACAUUGGCUGCUUAUGGUGACAAUUGAGA | |
| | GAUUGUUGCCAUAUAGCUAUUGGAUUGGCCAUCCGGUGUCUAAUAGAGCGAUAAUCUACCAGUUUGU | |
| | UGGAUUUGUACCACUCAACUUUUCAACACUCAAGACACUCAACUACAUACUAGCACUCAACACAGUA | |
| | AA | |
| 295 | UUAAAACAGCCUGUGGGUUGUACCCACCCACAGGGCCCACUGGGCGCUAGCACACUGGUAUCACGGU | echovirus11 |
| | ACCUUUGUGCGCCUGUUUUAUACCCCCUUCCCGCAACCGCAAAUUUAGAAGCAAAGCUAACCCGAUC | |
| | GAUAGCGGAUGCGCAUGCCAGCCGCAUUUUGAUCAAGUACUUCUGUUUCCCCGGACCGAGUAUCAAU | |
| | AGACUGCUCACGCGGUUGAAGGAGAAAACGUCCGUUACCCGACCAACUACUUCGAGAAACCUAGUAA | |
| | CAUCAUGAAUGUUGCAGGGCGUUUCGAUCAGCACGACCCUGGUGUAGAUCAGGCUGAUGAGUCACCG | |
| | CAUUCCCCACGGGUGACCGUGGCGGUGGCUGCGUUGGCGGCCUGCCUAUGGGGUGACCCAUAGGACG | |
| | CUCUAAUACGGACAUGGUGCGAAGAGUCUAUUGAGCUAGUUGGUAGUCCUCCGGCCCCUGAAUGCGG | |
| | UUAAUCCUAACUGCGGACGACAUACCCCUAAUCCAAGGGGCAGUGUGUCGUAACGGGCAACUCUGCA | |
| | GCGGAACCGACUACUUUGGGUGUCCGUGUUUCCUUUUAUUUUUAUACUGGCUGCUUAUGGUGACAAU | |
| | CUCAGAGUUGUUACCAUAUAGCUAUUGGAUUGGCCAUCCGGUGAGCAACAGAGCUGUCAUUUAUCAG | |
| | UUUGUUGGCUUUAUACCUCUAAAUCACACGGUUUUUUUUUUUUGGAACGCUUGUAUUCAUCUUAACC | |
| | CUCAAUAAGGCAAA | |
| 296 | UUAAAACAGCCUGUGGGUUGUCCCCACCCACAGGGCCCACUGGGCGCUAGCACACUGGUAUCCCGGU | echovirus12 |
| | ACCUUUGUGCGCCUGUUUUAUAUACCCUCCCCUCAGUAACCUAGAAGUUCAUCACAAAUGAUCAAUA | |
| | GUUAGCUCAACAAACCAGUUGAGCCUAGAUCAAGCACUUCUGUUACCCCGGGCUGAGUAUCAAUAAG | |
| | CUGUUGACACGGCUGAAGGAGAAAACGCCCGUUACCCGACCAGCUACUUCGGAGAACCUAGUAUCAC | |
| | CAUAGAGGUUGCGUAGCGUUUCGCUCCGCACAACCCCAGUGUAGAUCAGGUCGAUGAGUCACCGCGU | |
| | UCCCCACAGGCGACUGUGGCGGUGGCUGCGUUGGCGGCCUGCCCAUGGGGUUACCCAUGGGACGCUU | |
| | CAAUACUGACAUGGUGUGAAGAGUUGACUGAGCUAGCUGGUAGUCCUCCGGCCCCUGAAUGCGGCUA | |
| | AUCCUAACUGUGGAGCAAGUGCCCACAACCCAGUGGGUGGCUUGUCGUAAUGGGCAACUCUGCAGCG | |
| | GAACCGACUACUUUGGGUGACCGUGUUUCUCUUUAUUCUUAUAUUGGCUGCUUAUGGUGACAAUCUC | |
| | AGAGUUGUUACCAUAUAGCUAUUGGUUUGGCCAACCAGUGACUAACAGAGCAAUUAUAUAUCUUUUC | |
| | AUUGGAUUUAUACCUAUAAACAACACCAGUUAUACCACUUUGUGUUACAUUAUUGGCUUGAACUCGA | |
| | AAAG | |
| 297 | UUAAAACAGCCUGUGGGUUGUACCCACCCACAGGGCCCACUGGGCGCCAGCACACUGGUAUCACGGU | echovirus13 |
| | ACCCUUGUGCGCCUGUUUUAUCCACCCCUCCCCGAAGUAACUUAGAAGCUCUAAAUAAGCGGUCAGC | |
| | AGCCAGCCCAGCACACCAGCUGGGUUUUGACCAAGUACUUCUGUUACCCCGGACCGAGUAUCAAUAG | |
| | GCUGCACACGCGGCUGAAGGAGAAAACGUUCGUUACCCGGCCAUCUACUUCGAGAAACCUAGUACCA | |
| | CUAUGAAAGUUGCGAAGCGUUUCGCUCCACACAACCCCAGUGUAGAUCAGGCUGAUGAGUCACCGCA | |
| | UUCCCCACAGGUGACUGUGGCGGUGGCUGCGUUGGCGGCCUGCCCAUGGGGCAACCCAUGGGACGCU | |
| | UCAAUACCGACAUGGUGCGAAGAGUCUAUUGAGCUAGAUGGUAGUCCUCCGGCCCCUGAAUGCGGCU | |
| | AAUCCUAACUGCGGAGCAGGUGCACACAAACCAGUGAGUAGCCUGUCGUAAUGGGCAACUCUGCAGC | |
| | GGAACCGACUACUUUGGGUGUCCGUGUUUCUUUUUAUUCUUAUAUUGGCUGCUUAUGGUGACAAUUG | |
| | AAAGAUUGUUACCAUAUAGCUAUUGGAUUGGCCAUCCGGUGACUAGCAGAGCACUGAUAUACCUAUU | |
| | UGUGGGCUUUGUACCACUCAACUUUGUCAGCUACACCACAUUGCUAUAUAUUGUCCAACUGAACGCA | |
| | AGAAG | |
| 298 | UUAAAACAGCCUGUGGGUUGUUCCCACCCACAGGGCCCACAGGGCGCAAGCACACUGGUAUCACGGU | echovirus14 |
| | ACCUUUGUGCGCCUGUUUUAUCUACCCCCCCCAAAGUAACGUAGAAGCCUGAACAAAGUGGUCACUA | |
| | GUAAGCUCGGUAAGCCAACCGAGGUUAGACCAAGCACUUCUGUCUCCCCGGACUGAGUAUCAAUAGG | |
| | CUGCUAACGCGGCUGAAGGAGAAAACGUUCGUUACCCGACCAGCUACUUCGAGAAACCUAGUACCAC | |
| | CAUGGAUGUUGCGCGGCGUUUCGUUCAACACAACCCCAGUGUAGAUCAGGUCGAUGAGUCACCGCUC | |
| | UCCCCACGGGCGACCGUGGCGGUGGCUGCGUUGGCGGCCUGCCCAUGGGGCAACCCAUGGGACGCUU | |
| | CAAUACUGACAUGGUGCGAAGAGUCUAUUGAGCUAGCUGGUAGUCCUCCGGCCCCUGAAUGCGGCUA | |
| | AUCCUAACUGUGGAGCAGGCGCUCGCAAACCAGCGAGUAACCUGUCGUAACGGGCAACUCCGCAGCG | |
| | GAACCGACUACUUUGGGUGUCCGUGUUUCCUUUUAUUCCUACCUUGGCUGCUUAUGGUGACAAUUGA | |
| | GAGAUUGUUACCAUAUAGCUAUUGGAUUGGCCAUCCGGUAAGCAACAGAGCAGUAAUUUACUUGUUC | |
| | GUUGGUUUCAUACCACGUGACUUUAAAGUCUUACAAACACUAAACCUCAUAUUAUCAUUUAAUAGUG | |
| | CAAA | |
| 299 | UUAAAACAGCCUGUGGGUUGUACCCACCCACAGGGCCCACUGGGCGCUAGCACUCUGGUAUUACGGU | echovirus15 |
| | ACCCUUGUGCGCCUGUUUUAUAUACCCCGCCCCAAGCAAACGCUAGAUGUAACGCACUUAUGAUCAA | |
| | UAGCAGGCGUGGCACUCCAGCCACGUUAUGAUCAAGCACUUCUGUCUCCCCGGACCGAGUAUCAAUA | |
| | GACUGCUCACGCGGUCGAAGGAGAAAACGUUCGUUACCCGACCAGCUACUUCGAGAAACCUAGUAAC | |
| | UCCAUGGAGGUUGCAGAGUGUUUCGUUCAGCACUUCCCCCGUGUAGAUCAGGCUGAUGAGUCACCGC | |
| | GUUCCUCACGGGCGACCGUGGCGGUGGCUGCGUUGGCGGCCUGCCUAUGGGUAUACCCAUAGGACGC | |
| | UCUAAUACUGACAUGGUGCGAAGAGUCUAUUGAGCUAGUUGGUAGUCCUCCGGCCCCUGAAUGCGGC | |
| | UAAUCCUAACUGCGGAGCACAUACCCCCAACCCAGGGGGCAGUGUGUCGUAACGGGUAACUCUGCAG | |
| | CGGAACCGACUACUUUGGGUGUCCGUGUUUCCUUUUAUUCUUACAUUGGCUGCUUAUGGUGACAAUU | |
| | GAGAGAUUGUUACCAUAUAGCUAUUGGAUUGGCCAUCCAGUGACUAACAGAGCUAUUAUUUACCUUU | |
| | UUGUUGGCUUCGUAUCACUUGGUUUAAAAGAGGUUAGUACUUUAUAUUGCAUUAUAUUACUAAACAC | |
| | GAGAAA | |
| 300 | UUAAAACAGCCUGUGGGUUGUUCCCACCCACAGGGCCCACUGGGCGCAAGCACACUGGUAUUACGGU | echovirus16 |
| | ACCUUUGUGCGCCUGUUUUACAUCCCCUCUCCCAUUAUAACUUAGAAGCACAUACAAAACGACCAAU | |
| | AGGUGGUCCAGCACUCCAGCUGGGCUCUGGUCAAGUACUUCUGUUACCCCGGACUAAGUAUCAAUAA | |
| | GCGGCUAACGCGGCUGAAGGAGAAAGCGUCCGUUACCCGGCCAACUACUUCGGGAAAUCUAGUAACA | |
| | CCAUGAAGGUUGCGCAGUGUUUCGCUCCGCACAAACCCAGUGUAGAUCAGGUCGAUGAGUCACCGCA | |
| | UUCCCCACAGGCGACUGUGGCGGUGGCUGCGUUGGCGGCCUGCCCAUGGGGCAACCCAUGGGACGCU | |
| | UCAAUACUGACAUGGUGCGAAGAAUCUACUGAGCUAGUUGGUAGUCCUCCGGCCCCUGAAUGCGGCU | |
| | AAUCCUAACUGCGGAGCACAUACCCCUAACCCAAGGGGCAGUGUGUCGUAACGGGCAACUCUGCAGC | |
| | GGAACCGACUACUUUGGGUGUCCGUGUUUCCUUUUAUUCUUAUACUGGCUGCUUAUGGUGACAAUUG | |
| | AGAAAUUGUUACCAUAUAGCUUUUGGAUUGGCCAUCCAGUGACUAACAGAGCUAUUAUUUACUUGUU | |
| | UGUUGGCUUUGUACCACUCAACCUUAGAGUUUUAAGGACUCUCAAUUUGGUUCUGCUAUCGAAUACA | |
| | ACAAA | |
| 301 | UUAAAACAGCCUGUGGGUUGUUCCCACCCACAGGGCCCAUUGGGCGCUAGCACUCUGGUAUUGCGGU | echovirus17 |
| | ACCUUUGUGCGCCUGUUUUAUAUUCCCUCCCCCUGACCGUAACUUAGAAGUAACACACACCGAUCAA | |
| | CAGUAAGCAUGGCACACCAGCCAUGUUUUGAUCAAGCACUUCUGUUAUCCCGGACUGAGUAUCAAUA | |
| | GACUGCUCACGCGGUUGAAGGAGAAAACGUUCGUUACCCGGCCAACUACUUCGAAAAACCUAGUAAC | |
| | ACCAUGGAAGUUGCAGAGUGUUUCGCUCAGCACUACCCCAGUGUAGAUCAGGUCGAUGAGUCACCGC | |
| | AUUCCCCACGGGCGACCGUGGCGGUGGCUGCGUUGGCGGCCUGCCUAUGGGGAAACCCAUAGGACGC | |
| | UCUAAUACAGACAUGGUGCGAAGAGUCUAUUGAGCUAGUUGGUAGUCCUCCGGCCCCUGAAUGCGGC | |
| | UAAUCCUAACUGCGGAGCACACACCCUCAAGCCAGGGGGCAGUGUGUCGUAACGGGCAACUCUGCAG | |
| | CGGAACCGACUACUUUGGGUGUCCGUGUUUCCCCUUACUCUUGUAUUGGCUGCUUAUGGUGACAAUU | |
| | UUGGAAUUGUUGCCAUAUAGCUAUUGGAUUGGCCAUCCGGUGACAAACAGAGCGAUUGUAUACCUAU | |
| | UCGUUGGACUUGUACCGCUCAACGUUACGAGUCUAACGACUUUAUACUACAUCAUCUGGUUGAACAC | |
| | AGCAAA | |
| 302 | UUAAAACAGCCUGUGGGUUGUACCCACCCACAGGGCCCACUGGGCGCUAGCACUCUGGUAUUACGGU | echovirus18 |
| | ACCUUUGUGCGCCUGUUUUACAUCCCUUCCCAAUCGUAAUUUAGAAGCCAAACACUACGAUCAAUAG | |
| | UGGGCGUGGCACGCCAGCCAUGUCUCGAUCAAGCACUCCUGUUUCCCCGGGCCGAGUAUCAAUAGAC | |
| | UGCUCACGUGGUUGAAGGAGAAAACGCUCGUUAUCCGGCUAACUACUUCGAGAAACCUAGUAACACC | |
| | AUGGAAGUUGCAGAGUGUUUCGUUCAGCACUUCCCCAGUGUAGAUCAGGCCGAUGAGUCACCGCGUU | |
| | CCCCACGGGUGACCGUGGCGGUGGCUGCGUUGGCGGCCUGCCCAUGGGGUAACCCAUGGGACGCUCU | |
| | AAUAUGGACAUGGUGUGAAGAGUCUAUUGAGCUAGUUGGUAGUCCUCCGGCCCCUGAAUGCGGCUAA | |
| | UCCUAACUGCGGAGCAUGUACCUCCAAUCCAGGAGGUGGCGUGUCGUAAUGGGCAACUCUGCAGCGG | |
| | AACCGACUACUUUGGGAGUCCGUGUUUCCUUUUAUUCUUACACUGGCUGCUUAUGGUGACAAUUGAG | |
| | AGAUUGUUACCAUAUAGCUAUUGGAUUGGCCAUCCGGUGACUAACAGAGCAGUUAUCUACCAGUUCG | |
| | UUGGUUUCAUUCCAUUGUGUACUCGCGAAUACAACACUCUUCUUUUAAUACUAGAUUUAAACAAAGC | |
| | AAA | |
| 303 | UUAAAACAGCCUGUGGGUUGUACCCACCCACAGGGCCCAAUGGGCGCUAGCACUCCGGUAUUACGGU | echovirus19 |
| | ACCCUUGUGCGCCUGUUUUAUUUCCCCUUCCCCAACUGAAACUUAGAAGAAAUACACAAUGAUCGAC | |
| | AGUAAGCGUGGCGCACCAGCCAUGUUUUGAUCAAGCACUUCUGUUUCCCCGGACCAAGUAUCAAUAG | |
| | ACUGCUCACGCGGUUGAAGGAGAAAGCGUUCGUUAACCGGCUAACUACUUCGAGAAACCCAGUAACA | |
| | CCAUGAAAGUUGCAGAGUGUUUCGCUCAGCACUCCCCCAGUGUGGAUCAGGUCGAUGAGUCACUGCG | |
| | UUCCCCACGGGUGACCGUGGCAGUGGCUGCGUUGGCGGCCUGCCUGUGGGGCAACCCACAGGACGCU | |
| | CUAAUACGGACAUGGUGUGAAGAGUCUAUUGAGCUAGUUAGUAGUCCUCCGGCCCCUGAAUGCGGCU | |
| | AAUCCUAACUGUGGAGCACAUGCCCCUAACCCAAGGGGUGGUGUGUCGUAAUGGGUAACUCCGCAGC | |
| | GGAACCGACUACUUUGGGUGACCGUGUUUCCUUUUAUUUUUACAUUGGCUGCAUAUGGUGACGAUUG | |
| | AGAGAUUGUUGCCAUAUAGCUAUUGGAUUGGCCAUCCAGUGUCUAACAGAGCUAUUGUAUAUCUUUU | |
| | UGUUGGAUUUGUACCACUUGACUUGAAAGAGGUCAGGACACUACAAUACAUUAUACUGCUGAAUACU | |
| | GCAAA | |
| 304 | UUAAAACAGCCUGUGGGUUGUUCCCACCCACAGGGCCCAUUGGGCGCUAGCACUCUGGUAUUACGGU | echovirus20 |
| | ACCUUUGUGCGCCUGUUUUAUGUCCCCUCCCCCAAUCGCAACUUAGAAGCAACACACACUGAUCAAC | |
| | AGUAAGCGUGGCAUACCAGCCACGUUUUGAUCAAGCACUUCUGUUACCCCGGACUGAGUAUCAAUAG | |
| | ACUGCUCACGCGGUUGAAGGAGAAAGCGUUCGUUAUCCGGCCAACUACUUCGAGAAACCUAGUAACA | |
| | CCGUGAAAGUUGCAGAGUGUUUCGCUCAGCACUACCCCAGUGUAGAUCAGGUCGAUGAGUCACCGCA | |
| | UUCCCCACGGGCGACCGUGGCGGUGGCUGCGCUGGCGGCCUGCCUACGGGGAAACCCGUAGGACGCU | |
| | CUAAUACAGACAUGGUGCGAAGAGUCUAUUGAGCUAGUUGGUAGUCCUCCGGCCCCUGAAUGCGGCU | |
| | AAUCCUAACUGCGGAGCACACACCCCCAAGCCAGGGGGCAGUGUGUCGUAACGGGUAACUCUGCAGC | |
| | GGAACCGACUACUUUGGGUGUCCGUGUUUCAUUUUAUUCCUAUGCUGGCUGCUUAUGGUGACAAUUG | |
| | ACAGAUUGUUACCAUAUAGCUAUUGGAUUGGCCAUCCGGUGACUAAUAGAGCCAUUAUAUACCACUU | |
| | UGUUGGGUUUAUACCACUCAACUUGAAAGAGGUCAAAACACUACAGCUCAUCAUUAAAUUGAACACA | |
| | ACAAA | |
| 305 | UUAAAACAGCCUGUGGGUUGUUCCCACCCACAGGGCCCACCGGGCGCUAGCACACUGGUAUCGCGGU | echovirus21 |
| | ACCUUUGUGCGCCUGUUUUAUUUACCCUGCCCUGAUGUAACUUAGAAGCAUGACACCAACGAUCAAU | |
| | AGCAGGCUCAAUGCACCAAUUGGGUCAAGAUCAAGCACUUCUGUUACCCCGGACUGAGUAUCAAUAA | |
| | GCUGCUAGCGCGGCUGAAGGAGAAAAAGUUCGUUACCCGGCCAGCUACUUCGAGAAACCUAGUAUCA | |
| | CCAUGAAAGUUGCGUGGCGUUUCGCUCCGCACAACCCCAGUGUAGAUCAGGUCGAUGAGUCACCGCA | |
| | UUCCCCACGGGCGACCGUGGCGGUGGCUGCGUUGGCGGCCUGCCCGUGGAGCAAUCCAUGGGACGCU | |
| | AUAAUACAGACAUGGUGUGAAGAGUCUAUUGAGCUAGCUGGUAGUCCUCCGGCCCCUGAAUGCGGCU | |
| | AAUCCUAACUGCGGAGCAGGUACCCACGAACCAGUGGGCAGUCUGUCGUAAUGGGCAACUCCGCAGC | |
| | GGAACCGACUACUUUGGGUGUCCGUGUUUCCUUUUAUUUCAACUUGGCUGCUUAUGGUGACAAUUGA | |
| | GAGAUUGUUACCAUAUAGCUAUUGGAUUGGCCAUCCGGUGACUAACAGAGCUAUCAUAUACCUUUUU | |
| | AUUGGCUUUGUGCCACUUACUCUGAAAGAGGUCAAGACUCUGCAUUAUAUCAUACUAUUGAAUGCUA | |
| | UAAA | |
| 306 | UUAAAACAGCCUGUGGGUUGCACCCACCCACAGGGCCCACAGGGCGCUAGCACUCUGGUAUCACGGU | echovirus24 |
| | ACCUUUGUGCGCCUGUUUUAUUACCCCUUCCCCAAUUGAAAAUUAGAAGCAAUGCACACCGAUCAAC | |
| | AGCAGGCGUGGCGCACCAGUCACGUCUCGAUCAAGCACUUCUGUUUCCCCGGACCGAGUAUCAAUAG | |
| | ACUGCUCACGCGGUUGAAGGAGAAAGUGUUCGUUAUCCGGCUAACCACUUCGAGAAACCCAGUAACA | |
| | CCAUGAAAGUUGCAGGGUGUUUCGCUCAGCACUUCCCCAGUGUAGAUCAGGUCGAUGAGUCACCGCG | |
| | UUCCCCACGGGCGACCGUGGCGGUGGCUGCGUUGGCGGCCUGCCUAUGGGUUAACCCAUAGGACGCU | |
| | CUAAUACAGACAUGGUGCGAAGAGUUUAUUGAGCUGGUUAGUAUCCCUCCGGCCCCUGAAUGCGGCU | |
| | AAUCCUAACUGCGGAGCACGUGCCUCCAAUCCAGGGGGUUGCAUGUCGUAACGGGUAACUCUGCAGC | |
| | GGAACCGACUACUUUGGGUGUCCGUGUUUCCUUUUAUUCUUAUACUGGCUGCUUAUGGUGACAAUCG | |
| | AGGAAUUGUUACCAUAUAGCUAUUGGAUUGGCCAUCCGGUGUCUAACAGAGCGAUUAUAUACCUCUU | |
| | UGUUGGAUUUAUGCAGCUCAAUACCACCAACUUUAACACAUUGAAAUAUAUCUUAAAGUUAAACACA | |
| | GCAAA | |
| 307 | UUAAAACAGCCUGUGGGUUGUACCCACCCACAGGGCCCACUGGGCGCUAGCACUCUGGUACUACGGU | echovirus25 |
| | ACCUUUGUGUGCCUGUUUUAUACCCCUCCCCCUACUGAAACUUAGAAGCAAUUCAUACCGAUCAAUA | |
| | GUGGGCGUGGCACACCAGCCGUGUCUAGAUCAAGCACUCCUGUUUCCCCGGACCGAGUAUCAAUAGA | |
| | CUGCUCACGCGGUUGAAGGAGAAAACGUUCGUUAUCCGGCUAACUACUUCGAAAAACCUAGUAACAC | |
| | CAUGAAAGUUGCGGAGUGUUUCACUCAGCACUUCCCCAGUGUAGAUCAGGUCGAUGAGUCACCGCAU | |
| | UCCUCACGGGCGACCGUGGCGGUGGCUGCGCUGGCGGCCUGCCUAUGGGGUGACCCAUAGGACGCUC | |
| | UAAUACAGACAUGGUGCGAAGAGUCUAUUGAGCUAGUUAGUAGUCCUCCGGCCCCUGAAUGCGGAUA | |
| | AUCCUAACUGUGGAGCAGAUACCCACGAACCAGUGGGCAGUCUGUCGUAACGGGCAACUCCGCAGCG | |
| | GAACCGACUACUUUGGGUGUCCGUGUUUCCUUUUAUUCCAAAUCUGGCUGCUUAUGGUGACAAUUGA | |
| | GAGAUUGUUGCCAUAUAGCUAUUGGAUUGGCCAUCCGGUGAAUAAUAGAGCGAUAAUAUAUUUGUUU | |
| | GUUGGAUUCGUGCCACUUAGUCUGAAAGUUUUGAGAACACUCAACUACGUUUUAUUGCUGAAUAGUG | |
| | CAAG | |
| 308 | UUAAAACAGCCUGUGGGUUGUUCCCACCCACAGGGCCCACUGGGCGCUAGCACACCGGUAUCACGGU | echovirus26 |
| | ACCCUUGUGCGCCUGUUUUAUCCACCUCCCCCGUUGUAACUUAGAAGCUCAAUACAAAUGGUCGAUA | |
| | GCCAGCCCGAUACGCCAAUUGGGUUUUGACCAAGCACUUCUGUUACCCCGGACUGAGUACCAAUAGG | |
| | CUGCCAAAGCGGCUGAAGGGGAAAACGUUCGUUACCCCACCAACUACUUCGAGAAACCUAGUACCAC | |
| | CAUGAAGAUUGCGCAGCGUUUCGCUCAGCACAACCCCAGUGUAGAUCAGGUCGAUGAGUCACCGCAU | |
| | UCCCCACAGGCGACUGUGGCGGUGGCUGCGUUGGCGGCCUGCCCAUGGGGCUACCCAUGGGACACUU | |
| | CAAUAUCGACAUGGUGCGAAGAGUCUAUUGAGCUAGUUGGUAGUCCUCCGGCCCCUGAAUGCGGAUA | |
| | AUCCUAACUGCGGAGCAGGCACCCACAAGCCAGUGGGCAGCCUGUCGUAACGGGCAACUCUGCAGCG | |
| | GAACCGACUACUUUGGGUGUCCGUGUUUCUUUUUAUUCUUACACUGGCUGCUUAUGGUGACAAUUGA | |
| | AGAAUUGUUACCAUAUAGCUAUUGGAUUGGCCAUCCAGCGACUAACAGAGCUAUUAUCUUCUUGUUU | |
| | GUGGGGUACGUACCACUCAAUUUCACAGUAGUAAGGACUCUCAAUUUCAUUUUGUGCUUGAAUAAGG | |
| | CGAA | |
| 309 | UUAAAACAGCCUGUGGGUUGUACCCACCUACAGGGCCCACUGGGCGUCAGCACACUGGUAUCCCGGU | echovirus27 |
| | ACCUUUGUGCGCCUGUUUUAUAUUCCCCACCCCACGUUGUAACUUAGAAGCAAUGCAAUUUCGGUCA | |
| | GUAGUAGACGCGGCAACCCAGUCGUGUUUCGACCAAGCACUUCUGUAACCCCGGACCGAGUAUCAAU | |
| | AAGCUGCUCGCGCGGCUGAAGGAGAAACCGUUCGUUACCCGGCUAACUACUUCGAGAAGCCUACUAA | |
| | CACCAUGAACGUUGCAGAGUGUUUCACUCAGCACUUCCCCCGUGUAGAUCAGGUCGAUGAGUCACCG | |
| | CGAUCCCCACGGGCGACCGUGGCGGUGGCUGCGUUGGCGGCCUGCCUGUGGGGCAACCCACAGGACG | |
| | CUCUAAUACAGACAUGGUGCGAAGAGCCUAUUGAGCUAGUUAGUAGUCCUCCGGCCCCUGAAUGCGG | |
| | CUAAUCCUAACUGCGGAGCACAUGCCCACAAACCAGUGGGUGGUGUGUCGUAACGGGCAACUCUGCA | |
| | GCGGAACCGACUACUUUGGGUGUCCGUGUUUCUUUUUAUUCUCAUACUGGCUGCUUAUGGUGACAAU | |
| | UGAAAGAUUGUUGCCAUAUAGCUAUUGGAUUAGCCAUCCGGUGUCAAACAGAGCAAUUAUUUACCUA | |
| | UUCGUUGGUUUUGCACCCUUGAAUUUUAAGGUUCUUAAAACACUUAAUCUCAUAUUAAAACUCAAUU | |
| | CAGCAAA | |
| 310 | UUAAAACAGCCUGUGGGUUGAUCCCACCCACAGGGCCCACUGGGCGCUAGCACUCUGGUAUCACGGU | echovirus29 |
| | ACCUUUGUGCGCCUGUUUUAUACUUCCUCCCCCAACUGCAACUUAGAAGUAACACAAACCGAUCAAC | |
| | AGUCAGCGUGGCACACCAGCCACGUUUUGAUCAAACACUUCUGUUACCCCGGACUGAGUAUCAAUAG | |
| | ACUGCUCACGCGGUUGAAGGAGAAAACGUUCGUUAUCCGGCCAACUACUUCGAGAAACCUAGUAACG | |
| | CCAUGGAAGUUGUGGAGUGUUUCGCUCAGCACUACCCCAGUGUAGAUCAGGUUGAUGAGUCACCGCA | |
| | UUCCCCACGGGUGACCGUGGCGGUGGCUGCGUUGGCGGCCUGCCCAUGGGGAAACCCAUGGGACGCU | |
| | CUUAUACAGACAUGGUGCGAAGAGUCUAUUGAGCUAGUUGGUAGUCCUCCGGCCCCUGAAUGCGGCU | |
| | AAUCCCAACUGCGGAGCAUACACUCUCAAGCCAGAGGGUAGUGUGUCGUAAUGGGCAACUCUGCAGC | |
| | GGAACCGACUACUUUGGGUGUCCGUGUUUCAUUUUAUUCCUAUACUGGCUGCUUAUGGUGACAAUUG | |
| | AGAGAUUGUUACCAUAUAGCUAUUGGAUUGGCCAUCCGGUGACUAACAGAGCUAUUAUAUAUCUUUU | |
| | UGUUGGGUUUAUACCACUUAGCUUGAAAGAGGUUAAAACUCUACAUUACAUUUUAAUACUGAACACC | |
| | GCAAA | |
| 311 | UUAAAACAGCCUGUGGGUUGUUCCCACCCACAGGGCCCAUGGGCGCUAGCACACUGGUAUCGUGGUA | echovirus31 |
| | CCUUUGUGCGCCUGUUUUAUACACCCUCCCCAUUUGUAACUUAGAAGUAAAGCAAUAUUGAUCAAUA | |
| | GCAAGCAUGGCACACCAGCCGUGUUCAGAUCAAGCACUUCUGUAACCCCGGACUGAGUAUCAAUAGG | |
| | CUGCCAAAGCGGCUGAAGGAGAAAACGUUCGUUAACCGGCCAACUACUUCGAGAAGCCCAGUAUCAC | |
| | CAUGAAAGUUGCAGAGCGUUUCGCUCAGCACUCCCCCCGUGUAGAUUAGGUCGAUGAGUCACCGCAU | |
| | CCCCCACAGGCGACUGUGGCGGUGGCUGCGUUGGCGGCCUACCUACGGGGCAACCCGUAGGACGCUC | |
| | UAAUACAGACAUGGUGAGAAGAGCCUAUUGAGCUAGUUGGUAGUCCUCCGGCCCCUGAAUGCGGAUA | |
| | AUCCUAACUGCGGAGCAUGUGCUCGCAAUCCAGCGAGAGGCAUGUCGUAACGGGCAACUCUGCAGCG | |
| | GAACCGACUACUUUGGGUGUCCGUGUUUCCUUUUAUUCCUUGUUUGGCUGCUUAUGGUGACAAUUGA | |
| | AAGAUUGUUGCCAUAUAGCUAUUGGGUCGGCCACCUGGUAUCUAACAGAGCAAUUAUUUACUUGUUU | |
| | GUUGGUUUUGUACCCUUAAAUCUCAAGGUUCUUGAAACCCUUAACUACACAUUGAAACUCAAUUCAU | |
| | UGAA | |
| 312 | UGUGGGUUGUUCCCACCCACAGGGCCCACUGGGCGCUAGUACACUGGUACCAAGGUACCUUUGUACG | echovirus32 |
| | CCUGUUUUAUACCCCCUCCCCGAUCCGAAACUUAGAAGUAAAGCAAACCCGAUCAAUAGUAGGUGCG | |
| | ACACUCCAGUCGUAUCUCGAUCAAGCACUUCUGUUUCCCCGGACUGAGUAUCAAUAGACUGCCCACG | |
| | CGGUUGAAGGAGAAAACGUCCGUCAUCCGGCUAACUACUUCGAGAAACCCAGUAGCACCAUGGAAGU | |
| | UGCAGAGUGUUUCGCUCAGCACUAACCCCGUGUAGAUCAGGCUGAUGAGUCACCGCAUUCCCCACGG | |
| | GUGACCGUGGCGGUGGCUGCGUUGGCGGCCUGCCUAUGGGGCAACCCAUAGGACGCUCUAAUACGGA | |
| | CAUGGUGUGAAGAGUCUAUUGAGCUAGUUAGUAAUCCUCCGGCCCCUGAAUGCGGCUAAUCCUAACU | |
| | GCGGAGCACAUACUCCUACUCCAAGGAGCAGUGUGUCGUAACGGGCAACUCUGCAGCGGAACCGACU | |
| | ACUUUGGGUGUCCGUGUUUCCUAUUAACAUAUCACUGGCUGCUUAUGGUGACAAUUGAAAGAUUGUU | |
| | ACCAUAUAGCUAUUGGAUUGGCCAUCCGGUGUCAAACAGAGCAAUAGUGUACCUAUUCGCUGGGUUU | |
| | AUCCCAUUGACUGUUAAAGAUAUACAAACUCUCUACUUGAUAAUACAACUAAACACUGUAAA | |
| | UUAAAACAGCCUGUGGGUUGAUCCCACCCACAGGGCCCAUUGGGCGCUAGCACUCUGGUAUCACGGU | echovirus33 |
| 313 | ACCCUUGUGCGCCUGUUUUAUGUCCCUUCCCUCAACUGUAACUUAGAAGUAACGCACACCGAUCAAC | |
| | AGUCAGCGUGGCACACCAGCCAUGUUUUGAUCAAGCACUUCUGUUACCCCGGACCGAGUAUCAACAG | |
| | ACUGCUCACGCGGUUGAAGGAGAAAGUGUUCGUUAUCCGGCCAACUACUUCGAAAAACCUAGUAACA | |
| | CCAUGGAAGUUGCAGAGUGUUUCGCUCAGCACUACCCCAGUGUAGAUCAGGUCGAUGAGUCACCGCA | |
| | UCCCCCACGGGCGACCGUGGCGGUGGCUGCGUUGGCGGCCUGCCUAUGGGGGAACCCAUAGGACGCU | |
| | CUAAUACAGACAUGGUGCGAAGAGUCCAUUGAGCUAGUUGGUAGUCCUCCGGCCCCUGAAUGCGGCU | |
| | AAUCCUAACUGCGGAGCACACACCUUCAAGCCAGAGGGCAGUGUGUCGUAACGGGCAACUCUGCAGC | |
| | GGAACCGACUACUUUGGGUGUCCGUGUUUCAUUUUAUUCUUAUACUGGCUGCUUAUGGUGACAAUUG | |
| | AGAGAUUGUUACCAUAUAGCUAUUGGAUUGGCCAUCCAGUGACUAGCAGAGCUAUUAUAUACCUCUU | |
| | UGUUGGGUUUAUACCACCUAAUUUGAAAGAAGUUAAAACAUUAGAAUUCAUUAUUAAAUUGAAUACA | |
| | UUUAA | |
| 314 | UUAAAACAGCCUGUGGGUUGUACCCACCCACAGGGCCCACUGGGCGCUAGCACACUGGUAUCACGGU | enterovirusB69 |
| | ACCUUUGUGCGCCUGUUUUAUACCCCCUACCCCAACUUUAAACUUAGAAGCAAAGCAAACCCGAUCA | |
| | AUAGCAGAUGUAGCAAGCCAGUUGCAUCUUGAUCAAGUACUUCUGUCUCCCCGGACUGAGUAUCAAU | |
| | AGACUGCUCACGCGGUUGAAGGAGAAAACGUUCGUUACCCGACCAACUACUUCGAGAAACCUAGUAA | |
| | CACCAUGAAAGUAGCAGGGUGUUUCGAUCAGCACAACCCCAGUGUAGAUCAGGCUGAUGAGUCACCG | |
| | CAUCCCCCACGGGCGACCGUGGCGGUGGCUGCGCUGGCGGCCUGCCCAUGGGGCAACCCAUGGGACG | |
| | CUCUAAUAUGGACAUGGUGUGAAGAGUCUAUUGAGCUAGUUGGUAGUCCUCCGGCCCCUGAAUGCGG | |
| | CUAAUCCUAACUGCGGAGCACAUACCCCAAAGCCAAGGGGCAGUGUGUCGUAACGGGCAACUCUGCA | |
| | GCGGAACCGACUACUUUGGGUGUCCGUGUUUCCUUUUAUUCUCAAACUGGCUGCUUAUGGUGACAAU | |
| | CUCAGAGUUGUUACCAUAUAGCUAUUGGAUUGGCCAUCCGGUGAGCAACAGAGCAAUUGUCUACCAA | |
| | UUUGUUGGAUUUAUUCCACUCAAUCUAGCGGUUUUCAGAACAUUGAAUUAUAUUCUAACCCUCAACA | |
| | AGACGAA | |
| 315 | UUUAAAACAGCCUGUGGGUUGUUCCCACCCACAGGGCCCACGGGGCGCUAGCACACUGGUAUCACGG | enterovirusB73 |
| | UACCUUUGUGCGCCUGUUUUAUAUCCCCCCCCCCAAGUAAACCUUAGAAGCAAUACACACCUGGUCG | |
| | ACAGUGGGUGUGACAAUCCAGUCACAUCAUGACCAAGCACUUCUGUUUCCCCGGACCGAGUAUCAAU | |
| | AGGCUGCUCACGCGGCUGAAGGAGAAAGCGUUCGUUACCCGGCCAGCUACUUCGAGAAGCCUAGUAA | |
| | CACCAUGAAAGUUGCAGAGUGUUUCGUUCAGCACUUCCCCCGUGUAGAUCAGGCUGAUGAGUCACCG | |
| | CAUUCCCCACGGGCGACCGUGGCGGUGGCUGCGCUGGCGGCCUGCCUAUGGGACACCCCAUAGGACG | |
| | CUCUAAUACCGACAUGGUGCGAAGAGUCUAUUGAGCUAGCUGGUAGUCCUCCGGCCCCUGAAUGCGG | |
| | CUAAUCCCAACUGCGGAGCACGCACCCUCAAACCAGAGGGCAGCGUGUCGUAAUGGGCAACUCUGCA | |
| | GCGGAACCGACUACUUUGGGUGUCCGUGUUUCCUUUUAUUCUUACAUUGGCUGCUUAUGGUGACAAU | |
| | UGAGAGAUUGUUACCAUAUAGCUAUUGGAUUGGCCAUCCGGUGACAAACAAAGCUAUCAUAUAUUUA | |
| | UUCAUUGGUUACAUACCACUUACCCUGAAAGAGAUCAAGACCUUGCACUUUAUCUUAUCACUAAACA | |
| | CUGCAAA | |
| 316 | UUAAAACAGCCUGUGGGUUGUUCCCACUCACAGGGCCCACUGGGCGCUAGCACACUGGUAUCACGGU | enterovirusB74 |
| | ACCCUUGUGCGCCUGUUUUAUUCACCCCCCCCAAAUGUAACUUAGAAGCAUAACCCAAAUGGUCAAU | |
| | AGACAGCCCAGUACACCAGCUGGGUUUCGGCCAAGUACUUCUGUUACCCCGGACCGAGUAUCAAUAG | |
| | GCUGCUCACGCGGCUGAAGGAGAAAACGUUCGUUAUCCGGCCAAUUACUUCGAGAAACCUAGUAACA | |
| | CCAUGAAAGUUGCGCAGUGUUUCGCUCCGCACAACCCCAGUGUAGAUCAGGUCGAUGAGUCACCGCG | |
| | UUCCCCACGGGCGACCGUGGCGGUGGCUGCGUUGGCGGCCUGCCCAUGGGGCAACCCAUGGGACGCU | |
| | UCAAUACUGACAUGGUGCGAAGAGUCUAUUGAGCUAAUUGGUAGUCCUCCGGCCCCUGAAUGCGGCU | |
| | AAUCCUAACUGCGGAGCAGAUACCCACAUGCCAGUGGGCGGUCUGUCGUAACGGGCAACUCCGCAGC | |
| | GGAACCGACUACUUUGGGUGUCCGUGUUUCCUUUUAUUCUUAAUCUGGCUGCUUAUGGUGACAAUUG | |
| | AAAGAUUGUUGCCAUAUAGCUAUUGGAUUGGCCAUCCGGUGACAAACAGGGCGAUUAUUUAUUUGUU | |
| | UGUUGGUUUUGUUCCAUUAAACUAUAGAGUUUUGAAUACUCUAGAAUUUAUUAUAACACUCAACACA | |
| | ACAAA | |
| 317 | UUUAAAACAGUCCUGUGGGUUGUUACCCACCCACAGGGCCCACUGGGCGCCAGCACACUGGUAUCAC | enterovirusB75 |
| | GGUACCUUUGUGCGCCUGUUUUAUAUACCCUUCCCCUACUAAGUAACUUAGAAGCAAUUAAACAAAG | |
| | ACCAACAGUAAGCACAGCGCACCAGCUGUGUUUCGGUCAAGCACUUCUGUUACCCCGGACUGAGUAU | |
| | CAAUAGGCUACUCACGUGGCUGAAGGAGAAAACGUUCGUUACCCGACCAAUUACUUCGAGAAGCCUA | |
| | GUAACACCAUGGAGGUUGCGCGGCGUUUCGCUCCACACAACCCCAGUGCAGAUCAGGUCGAUGAGCC | |
| | ACCGCAUUCCCCACGGGCGACCGUGGCGGUGGCUGCGUUGGCGGCCUGCCCAUGGGGCAACCCAUGG | |
| | GACGCUUCAAUACUGACAUGGUGCGAAGAGUCUAUUGAGCUAAUUGGUAGUCCUCCGGCCCCUGAAU | |
| | GCGGCUAAUCCCAACUGCGGAGCAGAUACCCACGCGCCAGUGGGCAGUCUGUCGUAAUGGGCAACUC | |
| | UGCAGCGGAACCGACUACUUUGGGUGUCCGUGUUUCCUUUAUUCUUAUACUGGCUGCUUAUGGUGAC | |
| | AAUUGAGAGAUUGUUACCAUAUAGCUAUUGGAUUGGCCAUCCGGUGACGAGCAGAGCUAUUAUUUAC | |
| | CUAUUUGUUGGUUUUAUACCAUUGAACUAUAAAGUUUUAAAAACCUUAAACUUCAUCUUAGUACUCA | |
| | AUACAGCAAA | |
| 318 | UUAAAACAGCCUGUGGGUUGUACCCACCCACAGGGCCCAUUGGGCGCUAGCACACUGGUAUUCCGGU | enterovirusB77 |
| | ACCCUUGUGCGCCUGUUUUAUCUACCCUUCCCUACUGCAACUUAGAAGUUAAACAGUCACGGUCGAU | |
| | AGGCGGUUCAGUAAACCAACUGGAUCUUGACCAAGCACUUCUGUUACCCCGGACUGAGUAUCAAUAA | |
| | GCUGCUUACGUGGCUGAAGGAGAAAACGUUCGUUACCCGGCCAAUUACUUCGAGAAACCUAGUACCA | |
| | CCAUGAAGGUUGCGCAGCGUUUCGCUCCGCACAACCCCAGUGUAGAUCAGGUCGAUGAGUCACCGCA | |
| | CUCCUCACGGGCGACCGUGGCGGUGGCUGCGCUGGCGGCCUGCCCAUGGGGCAACCCAUGGGACGCU | |
| | UCAAUACUGACAUGGUGCGAAGAGUCUAUUGAGCUAAUUGGUAAUCCUCCGGCCCCUGAAUGCGGCU | |
| | AAUCCCAACUGCGGAGCAGGUACCCACACACCAGUGGGCAGCCUGUCGUAACGGGCAACUCUGCAGC | |
| | GGAACCGACUACUUUGGGUGUCCGUGUUUCCUUUUAUUCCUAUACUGGCUGCUUAUGGUGACAAUUG | |
| | AAAGAUUGUUACCAUAUAGCUAUUGGAUUGGCCAUCCAGUGACAAACAGAGCAAUUGUCUAUCUAUU | |
| | UGUCGGUUUUGUACCAAUAAAUUUCAAAGUCCUAAAAACCCUCAAUUUCAUAUUGUUACUUAACACA | |
| | ACAAG | |
| 319 | UUAAAACAGCCUGUGGGUUGUACCCACCCACAGGGCCCACUGGGCGCUAGCACACUGGUAUCCCGGU | enterovirusB79 |
| | ACCUUUGUGCGCCUGUUUUAUACCCCCUCCCCCAUAUCGUAACAUAGAAGCAAUGCACUCCCGGUCA | |
| | AUAGCUGACGUGACAAACCAGUUGCGUUUUGACCAAGCACUUCUGUAACCCCGGACCGAGUAUCAAU | |
| | AAGCUGCUUGCGCGGCUAAAGGAGAAACCGUCCGUUACCCGGCUAACUACUUCGAGAAACCUAGUAC | |
| | CACCAUGAACGUUGCGGAGUGUUUCGCUCAGCACUUCCCCCGUGUAGAUCAGGUCGAUGAGCCACCG | |
| | CAUUCCCCACGGGCGACCGUGGCGGUGGCUGCGUUGGCGGCCUGCCUGUGGGGCAACCCACAGGACG | |
| | CUCUAAUAUAGACAUGGUGUGAAGAGUCUAUUGAGCUAGUUAGUAGUCCUCCGGCCCCUGAAUGCGG | |
| | CUAAUCCUAACUGCGGAGCACAUACCCCUAAUCCAAGGGGCAGUGUGUCGUAACGGGCGACUCUGCA | |
| | GCGGAACCGACUACUUUGGGUGUCCGUGUUUCCUUUUAUCUUUUAUAUUGGCUGCUUAUGGUGACAA | |
| | UUGCAGAGUUGUUACCAUAUAGCUUUUGGAUUGGCCAUCCGGUGAGCAAUAGAGCUGUUAUUUAUCA | |
| | AUUUGUUGGAUUCGUCGCACUCAACCACACAGUCUUUAGAACACUCGGAUUUAUCUUGACCCUUAAU | |
| | ACGAGAAA | |
| 320 | UUAAAACAGCCUGUGGGUUGAUCCCACCCACAGGGCCCACUGGGCGUCAGCACUCUGGUAUCACGGU | enterovirusB80 |
| | ACCUUUGUGCGCCUGUUUUAUACUCCCUCCCCCCAACCGUAACUUAGAAGCAGCACUAACUGAUCAA | |
| | UAGCCAGCGCGGCAUGCCAGCCACGCUUCGAUCAAGCACUUCUGUUACCCCGGACUGAGUAUCAAUA | |
| | GACUGCUCACGCGGUUGAAGGAGAAAACGUUCGUCAUCCGGCCAACUACUUCGAGAAACCUAGUAAC | |
| | ACCAUGGAAGUUGCGGAGUGUUUCGCUCAGCACUACCCCAGUGUAGAUCAGGUCGAUGAGUCACCGC | |
| | AUUCCCCACAGGCGACUGUGGCGGUGGCUGCGUUGGCGGCCUGCCCAUGGGGAAACCCAUGGGACGC | |
| | UCUUAUAUAGACAUGGUGCGAAGAGUCUAUUGAGCUAGUUGGUAGUCCUCCGGCCCCUGAAUGCGGC | |
| | UAAUCCUAACUGCGGAGCAUACACCCUCAAACCAGAGGGCAGUGUGUCGUAAUGGGCAACUCUGCAG | |
| | CGGAACCGACUACUUUGGGUGUCCGUGUUUCACUUUAUUUCUAAAUUGGCUGCUUAUGGUGACAAUU | |
| | GAGAGAUUGUUACCAUAUAGCUAUUGGAUUGGCCAUCCGGUGACCAAUAGAGCUAUUAUAUAUCUCU | |
| | UUGUUGGGUUCAUACCACUUAGCUUGAAAGAGGUUAAAACUCUGCAUUACAUUAUACUAUUGAAUUC | |
| | CGCAAA | |
| 321 | UUAAAACAGCCUGUGGGUUGUACCCACCCACAGGGCCCAUUGGGCGCUUGCACACUGGUAUUCCGGU | enterovirusB81 |
| | ACCUUUGUGCGCCUGUUUUAUACAUCCUACCCCCGUAACUUAGAAGCAAGAUCACCCGAUCACCAGG | |
| | AAAUGUGGCAAACCAGUCACAUUCUGAUCAAGCACUUCUGUUUCCCCGGACUGAGUAUCAAUAGGCU | |
| | GUUCACACGGCUGAAGGAGAAAACGUUCGUUACCCGGCCAACUACUUCGAAAAACCUAGUAUCACCG | |
| | UGAACGUUGCAGAGUGUUUCGCUCAGCACUUUCCCCGUGUAGAUCAGGUCGAUGAGUCACCGCAUUC | |
| | CCCACGGGCGACCGUGGCGGUGGCUGCGUUGGCGGCCUGCCUGCGGGGCAACCCGUAGGACGCUCUA | |
| | AUACAGACACGGUGUGAAGAGUCUAUUGAGCUAGUUGGUAGUCCUCCGGCCCCUGAAUGCGGCUAAU | |
| | CCUAACUGCGGAGCGAGCGCUUGUAAUCCAACAGGUAGCUCGUCGUAACGGGUAACUCUGCAGCGGA | |
| | ACCGACUACUUUGGGUGACCGUGUUUCCUUUUAUUUCAUUACUGGCUGCUUAUGGUGACAAUCACAG | |
| | AGUUGUUACCAUAUAGCUAUUGGAUUGGCCAUCCGGUGUCAAAUAGAGCAAUUGUGUUUCUUUUUGU | |
| | UGGGUUUGUACCACUUGACUUGACCAGCUACAACACUCUGUGCUACAUUGUUAGAUUGAACACAGCA | |
| | AA | |
| 322 | UUAAAACAGCCUGUGGGUUGCACCCACCCGCAGGGCCCACUGGGCGCUAGUACACUGGUACCCCGGU | enterovirusB82 |
| | ACCCUUGUACGCCUGUUUUAUUUCUCCCUCCCCCAACCAGUAACCUAGAAGUAAAGCAAACCCGAUC | |
| | AAUAGCAGGUGCGACACACCAGUCGUAUCUUGAUCAAGCACUUCUGUAACCCCGGGCCGAGUAUCAA | |
| | UAGACUGCUCACGCGGUUGAGGGAGAAAACGCUCGUUAUCCGGCUGACUACUUCGAGAAACCCAGUA | |
| | ACACCAUAGAAGUUGCAGAGUGUUUCGCUCAGCACUUUCCCCGUGUAGAUCAGGUCGAUGAGUCACC | |
| | GCAUUCCCCACGGGUGACCGUGGCGGUGGCUGCGUUGGCGGCCUGCCUAUGGGGCAACCCAUGGGAC | |
| | GCUCUAAUACAGACAUGGUGUGAAGAGUCAAUUGAGCUAGUCAGUAUUCCUCCGGCCCCUGAAUGCG | |
| | GCUAAUCCUAACUGCGGAGCACGUGCCUCCAAUCCAGGGGGUGGCGUGUCGUAACGGGUAACUCUGC | |
| | AGCGGAACCGACUACUUUGGGUGUCCGUGUUUCCUUUUAUUCUUAUCUUGGCUGCUUAUGGUGACAA | |
| | UUGAAGGAUUGUUACCAUAUAGCUAUUGGAUUGGCCAUCCGGUGACUAACAGAGCAAUCAUAUACCA | |
| | AUUUGUUGGUUUUAUACCAUUGAAUUACAAAGUUCUAAUCACCCUUAAAUUCAUUUUAACACUUAAU | |
| | ACGUCAAA | |
| 323 | UUUAAAACAGCCUGUGGGUUGUUCCCACCCACAGGGCCCACUGGGCGCUAGCACUCUGGUAUCACGG | enterovirusB83 |
| | UACCCUUGUGCGCCUGUUUUAUAUCCCCACCCCAGUGGAACAUUAGAAGCAGCACACCUAUGAUCAA | |
| | UAGUGGGCGUGGCAUCCAGUCACGUUAUGAUCAAGCACUUCUGUGACCCCGGACUGAGUAUCAAUAG | |
| | ACUGCUCACGCGGUUGAAGGAGAAAACGUUCGUUACCCGGCCAGUUACUUCGAGAAACCCAGUAACA | |
| | CCAUGAAUGUUGCAGAGUGUUUCGCUCAGCACUUCCCCCGUGUAGAUCAGGUCGAUGAGCCACUGCA | |
| | UUCCCCACGGGCGACCGUGGCAGUGGCUGCGUUGGCGGCCUGCCUAUGGGGUCACCCAUAGGACGCU | |
| | CUAAUACUGACAUGGUGCGAAGAGUCUAUUGAGCUAACUGGUAGUCCUCCGGCCCCUGAAUGCGGCU | |
| | AAUCCUAACUGUGGAGCACAUUCCCCCAAUCCAGGGGGCAGUGUGUCGUAACGGGCAACUCUGCAGC | |
| | GGAACCGACUACUUUGGGUGUCCGUGUUUCUUUUUAUUCUCAUAUUGGCUGCUUAUGGUGACAAUUA | |
| | CAGAAUUGUUACCAUAUAGCUAUUGGAUUGGCCAUCCGGUGACUAACAGAGCAAUUAUUUAUCAAUU | |
| | CGUUGGAUUUGUCCCACUCGAUUUCAAAGCAGUCAAUACACUCUCAUAUAUCGUUACACUCAACACA | |
| | ACAAA | |
| 324 | UUAAAACAGCCUGUGGGUUGUUCCCACCCACAGGGCCCACCGGGCGCCAGCACACUGGUAUCACGGU | enterovirusB84 |
| | ACCCUUGUGCGCCUGUUUUAUCCACCCCUUCCCACAGUAACUUAGAAGCACACCAUGUAUACGGUCA | |
| | AUAGGCGGCUCAGUACACCAACUGGGCCACGACCAAGCACUUCUGUUACCCCGGACUGAGUAUCAAU | |
| | AAGCUGCUCACGCGGCUGAAGGAGAAAACGUUCGUUACCCGGCCAAUUACUUCGAGAAACCCAGUAC | |
| | CACCAUGAAGGUUGCGCAGCGUUUCGUUCCGCACAACCCCAGUGUAGAUCAGGUCGAUGAGUCACCG | |
| | CGUUCCUCACGGGUGACCGUGGCGGUGGCUGCGUUGGCGGCCUGCCCAUGGGGCAACCCAUGGGACG | |
| | CUUCAAUACUGACAUGGUGCGAAGAGUCUAUUGAGCUAAUUGGUAGUCCUCCGGCCCCUGAAUGCGG | |
| | CUAAUCCUAACUGCGGAGCAGAUACCCACAAACCAGUGGGCAGUCUGUCGUAACGGGCAACUCUGCA | |
| | GCGGAACCGACUACUUUGGGUGUCCGUGUUUCCUUUUAUCCUUACACUGGCUGCUUAUGGUGACAAU | |
| | UGAGAAAUUGUUACCAUAUAGCUAUUGGAUUGGCCAUCCGGUGACAAACAGAGCAGUUGUUUACCUA | |
| | UUUGUUGGUUUCGUACCGCUGAACCUUAAAGUUCUAAAGACCCUCAAUUUUAUCUUAGCACUUAACA | |
| | CAGCAAA | |
| 325 | UUAAAACAGCCUGUGGGUUGUUCCCACCCACAGGGCCCACCGGGCGCUAGCACACUGGUAUUGCGGU | enterovirusB85 |
| | ACCUUUGUGCGCCUGUUUUAUACACCCCUCCUUAAUGUAACUCAGAAGUACAACAUUACCGGUCAAC | |
| | AGAUGACUCAGUAAACCAACUGAGCCUCGACCAAGCACUUCUGUUUCCCCGGACCGAGUAUCAAUAG | |
| | GCUGCUCACGCGGCUGAAGGAGAAAGUGUUCGUUACCCGGCCAAUUACUUCGAGAAGCCCAGUACCA | |
| | CCAUGGAGGUUGCGCGGCGUUUCGCUCCGCACAUCCCCAGUGUAGAUCAGGCUGAUGAGUCACCGCG | |
| | UUCCCCACGGGUGACCGUGGCGGUGGCUGCGCUGGCGGCCUGCCCAUGGGGUAACCCAUGGGACGCU | |
| | UCAAUACUGACAUGGUGUGAAGAGCCUAUUGAGCUAAUUGGUAGUCCUCCGGCCCCUGAAUGCGGCU | |
| | AAUCCUAACUGCGGAGCAGAUACCCACACACCAGCGGGCAGUCUGUCGUAACGGGCAACUCUGCAGC | |
| | GGAACCGACUACUUUGGGUGUCCGUGUUUCCUUUUAUUUUUAUGCUGGCUGCUUAUGGUGACAAUUG | |
| | AGGAAUUGUUACCAUAUAGCUAUUGGAUUGGCCAUCCAGUGUCCAACAGAGCGAUCAUUUACCUGUU | |
| | CGUUGGGUUCGUACCACUAAACCACAAGUCACUCAGCACACUUAAUUACAUUAUAACUCUCAACACA | |
| | ACAAG | |
| 326 | UUAAAACAGCCUGUGGGUUGUACCCACCCACAGGGCCCACUGGGCGCUAGCACACUGGUAUUACGGU | enterovirusB86 |
| | ACCCUUGUGCGCCUGUUUUAUUUACCCCUCCCCCACGCAACCUAGAAGCAUGACUUAAGCGGUCAAU | |
| | AGAUGGCUCAGUAUGCCAACUGAGUUAUGACCAAGCACUUCUGUUACCCCGGACCGAGUAUCAAUAG | |
| | GCUGCUCACGCGGCUGAAGGAGAAAGUGUUCGUUACCCGGCCAGUUACUUCGAGAAACCUAGUAUCA | |
| | UCAUGAAGGUUGCGAGGCGUUUCGCUCCACACGACCCCGGUGUAGAUCAGGCUGAUGAGUCACCGCA | |
| | UUCCCCACGGGCGACCGUGGCGGUGGCUGCGCUGGCGGCCUGCCCAUGGGGCAACCCAUGGGACGCU | |
| | UCAAUAUUGACAUGGUGCGAAGAGUCUAUUGAGCUAACUGGUAGUCCUCCGGCCCCUGAAUGCGGCU | |
| | AAUCCUAACUGCGGAGCAGACACCCACAUGCCAGUGGGCAGUCUGUCGUAACGGGCAACUCUGCAGC | |
| | GGAACCGACUACUUUGGGUGUCCGUGUUUCCUUUUAUUCUUAUAUUGGCUGCUUAUGGUGACAAUUG | |
| | AAAGAUUGUUGCCAUAUAGCUAUUGGAUUGGCCAUCCAGUGACUAACAGAGCAAUUGUCUACCUGUU | |
| | CGUUGGUUACGUACCACUAAAUUAUAAAGUUUUGAACACACUUAAUUUUAUUAUAGCACUUAACACC | |
| | UUAAA | |
| 327 | UUAAAACAGCCUGUGGGUUGUUCCCACCCACAGGGCCCACUGGGCGCUAGCACACUGGUAUCACGGU | enterovirusB87 |
| | ACCCUUGUGCGCCUGUUUUAUACACCCUUCCCCUACUGUGACUUAGAAGCAAAACAAUGAUGACCAA | |
| | UAGCAAGCGCAGCACUCCAGCUGUGUUUCGGUCAAGCACUUCUGUUUCCCCGGACUGAGUAUCAAUA | |
| | GGCCACUCACGUGGCCGAAGGAGAAAACGUCCGUUACCCGGCCAAUUACUUCGAGAAACCCAGUAAC | |
| | ACCAUAGAGGUUGCGCAGCGUUUCGCUCCGCACUCCCCCAGUGUAGAUCAGGUCGAUGAGUCACCGC | |
| | GUUCCCCACGGGCGACCGUGGCGGUGGCUGCGUUGGCGGCCUGCCCAUGGGGUAACCCAUGGGACGC | |
| | UUCAAUACUGACAUGGUGCGAAGAGUCUAUUGAGCUAAUUGGUAGUCCUCCGGCCCCUGAAUGCGGC | |
| | UAAUCCUAACUGCGGAGCAGAUACCCACGCUCCAGUGGGCAGUCUGUCGUAACGGGUAACUCUGCAG | |
| | CGGAACCGACUACUUUGGGUGUCCGUGUUUCCUUUUAUUCUUAUAUUGGCUGCUUAUGGUGACAAUU | |
| | GAAAGAUUGUUACCAUAUAGCUAUUGGAUUGGCCAUCCGGUGUCUAAUAGAGCAAUUAUAUUCCUGU | |
| | UUGUUGGUUUUAUACCACUGAAUAUCACAGUUUACAAGACCCUCAAUUACAUCCUACGUUUAAACAA | |
| | GACAAA | |
| 328 | UUAAAACAGCCUGUGGGUUGAUCCCACCCACAGGGCCCACUGGGCGCUAGCACACUGGUAUUCCGGU | enterovirusB88 |
| | ACUUUUGUGCGCCUGUUUUAUACACCCUUCCCCUUCAUGUAACUUAGAAGUAAAACACAAACGGUCA | |
| | AUAGGUAGCUCAGCAAACCAGCUGAGCCUCGGCCAAGCACUUCUGUUACCCCGGACUGAGUAUCAAU | |
| | AAGCUGUUAACACGGCUGAAGGAGAAAACGUUCGUUAUCCGGCCAAUUACUUCGAGAAACCUAGUAA | |
| | CACCAUGAAGGUUGCGUAGCGUUUCGCUCCACACAACCCCAGUGUAGAUCAGGUCGAUGAGUCACCG | |
| | CUCUCCCCACGGGUGACCGUGGCGGUGGCUGCGUUGGCGGCCUGCCCAUGGAGCACUCCAUGGGACG | |
| | CUUCAAUACUGACAUGGUGUGAAGAGUCUAUUGAGCUAAUUGGUAGUCCUCCGGCCCCUGAAUGCGG | |
| | CUAAUCCUAACUGCGGAGCAGACACCCACACGCCAGUGGGCAGUCUGUCGUAAUGGGCAACUCUGCA | |
| | GCGGAACCGACUACUUUGGGUGUCCGUGUUUCCUUUUAUUCUUACAUUGGCUGCUUAUGGUGACAAU | |
| | CAAGGAAUUGUUACCAUAUAGCUAUUGGAUUGGCCAUCCGGUAUCUAACAGAGCAAUUGUUUACCUC | |
| | UUCACCGGUUUUGUACCAUUGAAUUGCAAUAGUCUCAACACCCUUUGCUAUAUCAUUAAAUUGAACU | |
| | CAGCAAA | |
| 329 | UUUAAAACAGCCUGUGGGUUGUUCCCaCUCACAGGGCCCACUGGGCGCUAGCACaCUGGUAUcACGG | enterovirusB93 |
| | UACCcUUGUGCGCCUGUUUUACCACCCCUUUCCCCAAAGCAAUUUAGAAGAAAGACUCAACCGGUCA | |
| | AUAGACAGCCUAGUACACCAACUAGGCCCUGACCAAGUACUUCUGUUUCCCCGGACUGAGUAUCAAU | |
| | AGGCUGCUUACGCGGCCGAAGGAGAAAACGUUCGUUACCCGGCCAAUUACUUCGAGAAACCCAGUAC | |
| | CACCAUGAAAGUUGCGCAGUGUUUCGCUCCGCACAACCCCAGUGUAGAUCAGGUCGAUGAGUCACCG | |
| | CAUCCCCCACAGGUGACUGUGGCGGUGGCUGCGUUGGCGGCCUGCCCAUGGGGCAACCCAUGGGACG | |
| | CUUCAAUACCGACAUGGUGCGAAGAGUCUAUUGAGCUAAUUGGUAGUCCUCCGGCCCCUGAAUGCGG | |
| | CUAAUCCUAACUGUGGAGCAGAUACCCACACACCAGUGGGCAGUCUGUCGUAAUGGGCAACUCCGCA | |
| | GCGGAACCGACUACUUUGGGUGUCCGUGUUUCCUUUUAUUCUCAUAUUGGCUGCUUAUGGUGACAAU | |
| | UGAAAGGUUGUUGCCAUAUAGCUAUUGGAUUGGCCACCCAGUGACUAACAGAGCUAUUGUUUAUUUG | |
| | UUUGUUGGAUUUGUACCAUUGAAUUACAAGGUCCUUAAUACUCUUAACUACAUCAUAAACCUCAAUA | |
| | CGACGCG | |
| 330 | UUAAAACAGCCUGUGGGUUGUUCCCACCCACAGGGCCCACUGGGCGCCAGCACACUGGUAUCACGGU | enterovirusB97 |
| | ACCUUUGUGCGCCUGUUUUAUCCACCCUUCCCCAAUGUAACUUAGAAGUAAAACUAGAAAGAUCAAU | |
| | AGCAAGCACAGUAUGCCAACUGUGUUAUGAUCAAGCACUUCUGUUACCCCGGACUGAGUAUCAAUAA | |
| | GCUGCUAAAGCGGCUGAAGGAGAAAACGUUCGUUACCCGGCCAACUACUUCGAGAAACCCAGUACCA | |
| | CCAUGAAGGUUGCGCAGUGUUUCGCUCCACACAAUCCCAGUGUAGAUCAGGUCGAUGAGUCACCGCG | |
| | UUCCCCACGGGCGACCGUGGCGGUGGCUGCGUUGGCGGCCUGCCCAUGGGAUGACCCAUGGGACGCU | |
| | UCAAUACUGACAUGGUGCGAAGAGUCUAUUGAGCUAGUUGGUAGUCCUCCGGCCCCUGAAUGCGGCU | |
| | AAUCCUAACUGCGGAGCAGAUACCCACACACCAGUGGGCAGUCUGUCGUAACGGGUAACUCUGCAGC | |
| | GGAACCGACUACUUUGGGUGUCCGUGUUUCUUUUUAUCCUCAAAUUGGCUGCUUAUGGUGACAAUUG | |
| | AUAAGUUGUUACCAUAUAGCUAUUGGAUUGGCCAUCCGGUGUCUAACAGAGCUAUUGUUUAUUUAUU | |
| | UGUUGGUUUCGUGCCACUAAAUUACAAAGUUUUUGAAACUCUCAACUUCAUUUUGACACUCAAUAAA | |
| | GCAAA | |
| 331 | UUAAAACAGCCUGUGGGUUGUUCCCACCCACAGGGCCCAAUGGGCGCCAGCACUCUGGUAUUACGGU | enterovirusB98 |
| | ACCUUUGUGCGCCUGUUUUAUAUCCCCUCCCCCAAUUGUAACUUAGAAGCAACACGCACCGACCAAC | |
| | AGUGGGCGUGGCACACCAGCCACGUCUUGGUCAAGCACAUCUGUUUCCCCGGACUGAGUAUCAAUAG | |
| | ACUGCUCACGCGGUCGAAGGAGAAAACGUUCGUUAUCCGGCCAACUACUUCGAGAAGCCCAGUAACA | |
| | CCAUGAAUAUUGCGAAGUGUUUCGCUCAGCACACUCCCAGUGUAGAUCAGGUCGAUGAGUCACCGCA | |
| | UUCCCCACGGGCGACCGUGGCGGUGGCUGCGUUGGCGGCCUGCCUAUGGGGCAACCCGUAGGACGCU | |
| | UCAAUAUGGACAUGGUGUGAAGAGUCUAUUGAGCUAGUUGGUAGUCCUCCGGCCCCUGAAUGCGGCU | |
| | AAUCCUAACCGCGGAGCAGGCACCCACAAACCAGUGGGCAGCUUGUCGUAACGGGUAACUCUGCGGC | |
| | GGAACCGACUACUUUGGGUGUCCGUGUUUCCUUUUAUUCUUAUACUGGCUGCUUAUGGUGACAAUUG | |
| | AGAGAUUGUUGCCAUAUAGCUAUUGGAUUGGCCAUCCGGUGACAAACAGAGCAAUUGUUUAUCUAUU | |
| | UGUUGGUUUUAUACCGUUAAGUUUUAAAGUCCUAAAAACCCUUAACUUGGUCAUACUACUCAACUCA | |
| | GCAAA | |
| 332 | UUAAAACAGUCUGUGGGUUGUUCCCACCCACAGGGCCCACGUGGCGCCAGCACACUGGUAUCUCGGU | enterovirusB100 |
| | ACCUUUGUGCGCCUGUUUUAUCCACCCUAAACCCCGUUGUAACUUAGAAGAUGAGCAACACUGAUCA | |
| | AUAGUGGGUGCAACAUGCCAGUUGCAUCACGAUCAAGCACUUCUGUAUCCCCGGACUGAGUAUCAAU | |
| | AGGCUGCUCACGCGGCUGAAGGAGAAAACGUUCGUUAUCCGGCCAGUUACUUCGAGAAACCUAGUAA | |
| | CAUCAUGGAGGUUGCGCAGUGUUUCGCUCCGCACACCCCCAGUGUAGAUCAGGUCGAUGAGUCACCG | |
| | CGUUCCCCACGGGCGACCGUGGCGGUGGCUGCGUUGGCGGCCUGCCCAUGGGAUAACCCAUGGGACG | |
| | CUUCAAUACUGACAUGGUGUGAAGAGUCUAUUGAGCUAACUGGUAGUCCUCCGGCCCCUGAAUGCGG | |
| | CUAAUCCUAACUGUGGAGCAGACACCCACAUGCCAGUGGGCAGUCUGUCGUAACGGGCAACUCCGCA | |
| | GCGGAACCGACUACUUUGGGUGUCCGUGUUUCCUUUUAUUCCUAUAUUGGCUGCUUAUGGUGACAAU | |
| | UGAAAGAUUGUUACCAUAUAGCUAUUGGAUUGGCCAUCCGGUGUCAAACAGAGCAAUCAUUUAUUUG | |
| | UUUGUUGGUUUUAUACCUCUGAACUUUAAAGUGCUAAAGACUCUCAAUUUUAUAUUAUAUCUCAACA | |
| | AAUCAGG | |
| 333 | UUAAAACAGCCUGUGGGUUGUUCCCACCCACAGGGCCCACUGGGCGCCAGCACUCUGGUAUUACGGU | enterovirusB101 |
| | ACCUUUGUGCGCCUGUUUUAUAUCCCCCUCCCCACUUGAAACUUAGAAGUGAAACACACUGAUCAAU | |
| | AGUUGACAUGGCAAGCCAGCCAUGUCUUGAUCAAGCACCUCUGUUUCCCCGGACCUAGUAUCAAUAG | |
| | ACUGCUCGCGCGGUUGAAAGAGAAAGCGUUCGUUACCCGGCUAACUACUUCGAGAAACUUAGUAACA | |
| | CCAUGGAAGUUGCGGAGUGUUUCGCUCAGCACAUCCCCAGUGUAGAUCAGGCCGAUGAGUCACCGCA | |
| | UUCCCCACGGGCGACCGUGGCGGUGGCUGCGUUGGCGGCCUGCCUGUGGGGCAACCCACAGGACGCU | |
| | CUAAUACAAACAUGGUGCGAAGAGUCUAUUGAGCUAGUUAGUAGUCCUCCGGCCCCUGAAUGCGGCU | |
| | AAUCCUAACUGCGGAGCACGUGCUUCCAAUCCAGGAGGUGGCGUGUCGUAACGGGUAACUCUGCAGC | |
| | GGAACCGACUACUUUGGGUGUCCGUGUUUCCUUUUAUUCUUGCAUUGGCUGCUUAUGGUGACAAUUG | |
| | AGAGAUUGUUACCAUAUAGCUAUUGGAUUGGCCAUCCGGUGACAAACAGAGCAAUUAUAUAUCUCUU | |
| | UGUUGGAUACAUUCCAUUGAACUACAAGGAGUUAAAAACCCUCAAUUAUAUUCUGUUCUUGAAUUCC | |
| | GCAAG | |
| 334 | UUAAAACAGCCUGUGGGUUGUUCCCACCCACAGGGCCCAUGGGGCGCCAGCACACUGGUAUUGCGGU | enterovirusB106 |
| | ACCUUUGUGCGCCUGUUUUAUUCACCCUUUCCCAAUGAAACUUAGAAGCUUAACUCAACCGGUCGAU | |
| | AGACAGCCCAGUACACCAACUGGGUUUUGACCAAGUACUCCUGUUUCCCCGGACUGAGUAUCAAUAA | |
| | GCUGCUCGCGCGGCUGAAGGAGAAAACGUUCGUUACCCGGCCAAUUACUUCGAGAAACCUAGUACCA | |
| | CCAUGAACGUUGCGCAGUGUUUCGCUCCGCACAACCCCAGUGUAGAUCAGGUCGAUGAGUCACCGCA | |
| | AUCCCCACGGGCGACCGUGGCGGUGGCUGCGCUGGCGGCCUGCCCAUGGGGCAUCCCAUGGGACGCU | |
| | UCAAUACUGACAUGGUGCGAAGAGUCUAUUGAGCUAAUUGGUAAUCCUCCGGCCCCUGAAUGCGGCU | |
| | AAUCCUAACUGCGGGGCAGACACCCACACGCCAGUGGGCAGUCUGUCGUAACGGGUAACUCCGCAGC | |
| | GGAACCGACUACUUUGGGUGUCCGUGUUUCCUUUUAUCUUUAUAUUGGCUGCUUAUGGUGACAAUUG | |
| | AAAGAUUGUUGCCAUAUAGCUAUUGGAUUGGCCAUCCGGUGUCUAAUAGAGCAAUUAUAUAUCUCUU | |
| | UGUUGGCUUCAUCCCGUUAAAUAAAGUGAGCUACCAGACCCUGUGCUAUAUUAUCAAAUUGAACACA | |
| | AUAAA | |
| 335 | UUUCCCCGGAAGGGUCUAACGCUAUGCUGUAUCCACGGCAGAAGUAUGACCUACCGUUAACCGGCCA | enterovirusB110 |
| | UGUACUUCGAGAAGCCUAGUACCAUUAUGAAAGUUGAUUGAUGUUACGUUCCCCAGCAACCCCAGUG | |
| | UAGUUCAGGUCGAUGAGUCUCGGCAUGCCCCACGGGCGACCGUGGCCGUGGCUGCGUUGGCGGCCUG | |
| | CCUACCACCUUAUGGUGUAGGACGUCAAGAUACUGACAUGGUGUGAAGAGCCUAUUGAGCUACGUGG | |
| | UAGUCCUCCGGCCCCUGAAUGCGGCUAAUCCUAACUCCGGAGCGUCCGCUAGCAAUCCCGCUAGAAG | |
| | GGCGUCGUAAUGCGCAAGUCUGGAGCGGAACCgACUACUUUGGGUGUCCGUGUUUCCUGUUUACUUA | |
| | UUGUUUGGCUGCUUAUGGUGACAACUUA | |
| 336 | UUAAAACAGCCUGUGGGUUGUUCCCACCCACAGGGCCCACUGGGCGCCAGCACACUGGUAUCACGGU | enterovirusB111 |
| | ACCCUUGUGCGCCUGUUUUAUAAACCCUUCCCCAAUGCAACUUAGAAGCUCAACUUUACCGGUCAAU | |
| | AGACAGUCCAGUACGCCAACUGGGCUUCGAUCAAGCACUUCUGUUACCCCGGACUAAGUAUCAAUAG | |
| | GCUGCUCACGCGGCUGAAGGAGAAAACGUUCGUUACCCGGCCAAUUACUUCGAGAAACCCAGUAACA | |
| | CUAUGGAGGUUGCGCAGUGUUUCGUUCCACACAACCCCAGUGUAGAUCAGGUCGAUGAGUCACCGCU | |
| | UUCCCCACGGGCGACCGUGGCGGUGGCUGCGUUGGCGGCCUGCCCAUGGGGCAACCCAUGGGACGCU | |
| | UCAAUACUGACAUGGUGUGAAGAGUCUAUUGAGCUAAUUGGUAUUCCUCCGGCCCCUGAAUGCGGCU | |
| | AAUCCUAACUGCGGAGCAGAUACUCACGAGCCAGUGGGCAGUCUGUCGUAACGGGUAACUCUGCAGC | |
| | GGAACCGACUACUUUGGGUGUCCGUGUUUCCUUUUAUUCAUAUAUUGGCUGCUUAUGGUGACAAUUG | |
| | AGAGAUUGUUACCAUAUAGCUAUUGGAUUGGCCAUCCGGUGACUAAUAGAGCAAUUGUUUACCUAUU | |
| | UGUUGGCUUUGUGCCGUUGAACUACAAGGUUUUAAAAACUAUUAACUACAUCCUAGUACUUAACACG | |
| | UUGAA | |
| 337 | UUUCAUCAUAAGCUACUUGGUCUUGCCAACCGGAAGAUAAUUUGGUUAUUUGUUGGUUUCACAAAUC | enterovirusB113 |
| | UGCGAACCUCUUCACUAAUUUUAUUAAUUGUCCUCAAAAUUCAAACAGG | |
| 338 | UUAAAACAGCUCUGGGGUUGCACCCGCCCCAGAGGCCCACGUGGCGGCUAGUACUCCGGUAUUGCGG | poliovirus1- |
| | UACCCUUGUACGCCUGUUUUAUACUCCCUUCCCGUAACUUAGACGCACAAAACCAAGUUCAAUAGAA | Sabin |
| | GGGGGUACAAACCAGUACCACCACGAACAAGCACUUCUGUUUCCCCGGUGAUGUUGUAUAGACUGCU | |
| | UGCGUGGUUGAAAGCGACGGAUCCGUUAUCCGCUUAUGUACUUCGAGAAGCCCAGUACCACCUCGGA | |
| | AUCUUCGAUGCGUUGCGCUCAGCACUCAACCCCAGAGUGUAGCUUAGGCUGAUGAGUCUGGACAUCC | |
| | CUCACCGGUGACGGUGGUCUAGGCUGCGUUGGCGGCCUACCUAUGGCUAACGCCAUGGGACGCUAGU | |
| | UGUGAACAAGGUGUGAAGAGCCUAUUGAGCUACAUAAGAAUCCUCCGGCCCCUGAAUGCGGCUAAUC | |
| | CCAACCUCGGGGCAGGUGGUCACAAACCAGUGAUUGGCCUGUCGUAACGCGCAAGUCCGUGGCGGAA | |
| | CCGACUACUUUGGGUGUCCGUGUUUCCUUUUAUUUUAUUGUGGCUGCUUAUGGUGACAAUCACAGAU | |
| | UGUUAUCAUAAAGCGAAUUGGAUUGGCCAUCCGGUGAAAGUGAGAUUCAUUAUCUAUCUGUUUGCUG | |
| | GAUUCGCUCCAUUGAGUGUGUUUACUCUAAGUACAAUUUCAACAGUUAUUUCAAUCAGACAAUUGUA | |
| | UCAUA | |
| 339 | UUAAAACAGCUCUGGGGUUGUUCCCACCCCAGAGGCCCACGUGGCGGCCAGUACACUGGUAUUGCGG | poliovirus2- |
| | UACCUUUGUACGCCUGUUUUAUACUCCCUUCCCCCGUAACUUAGAAGCACAAUGUCCAAGUUCAAUA | Lansing |
| | GGAGGGGGUACAAACCAGUACCACCACGAACAAGCACUUCUGUUCCCCCGGUGAGGCUGUAUAGGCU | |
| | GUUUCCACGGCUAAAAGCGGCUGAUCCGUUAUCCGCUCAUGUACUUCGAGAAGCCUAGUAUCACCUU | |
| | GGAAUCUUCGAUGCGUUGCGCUCAACACUCAACCCCAGAGUGUAGCUUAGGUCGAUGAGUCUGGACG | |
| | UUCCUCACCGGCGACGGUGGUCCAGGCUGCGUUGGCGGCCUACCUGUGGCCCAAAGCCACAGGACGC | |
| | UAGUUGUGAACAAGGUGUGAAGAGCCUAUUGAGCUACCUGAGAGUCCUCCGGCCCCUGAAUGCGGCU | |
| | AAUCCUAACCACGGAGCAGGCAGUGGCAAUCCAGCGACCAGCCUGUCGUAACGCGCAAGUUCGUGGC | |
| | GGAACCGACUACUUUGGGUGUCCGUGUUUCCUUUUAUUUUUACAAUGGCUGCUUAUGGUGACAAUCA | |
| | UUGAUUGUUAUCAUAAAGCAAAUUGGAUUGGCCAUCCGGUGAGAAUUUGAUUAUUAAAUUACUCUCU | |
| | UGUUGGGAUUGCUCCUUUGAAAUCUUGUGCACUCACACCUAUUGGAAUUACCUCAUUGUUAAGAUAU | |
| | CAUCACCACU | |
| 340 | UUAAAACAGCUCUGGGGUUGUACCCACUCCAGAGGCCCACGUGGCGGCUAGCACUCCGGUAUUACGG | poliovirus2- |
| | UACCUUUGUGCGCCUGUUUUAUACUCCCCUCCCGCAACUUAGAAGCACGAAACCAAGUUCAAUAGAA | Sabin |
| | GGGGGUACAAACCAGUACCACUACGAACAAGCACUUCUGUUUCCCCGGUGACAUUGCAUAGACUGCU | |
| | CACGCGGUUGAAAGUGAUCGAUCCGUUACCCGCUUGUGUACUUCGAAAAGCCUAGUAUCGCCUUGGA | |
| | AUCUUCGACGCGUUGCGCUCAGCACCCGACCCCGGGGUGUGGCUUAGGCUGAUGAGUCUGGACAUUC | |
| | CUCACCGGUGACGGUGGUCUAGGCUGCGUUGGCGGCCUACCUAUGGCUAACGCCAUAGGACGUUAGA | |
| | UGUGAACAAGGUGUGAAGAGCCUAUUGAGCUACAUAAGAGUCCUCCGGCCCCUGAAUGCGGCUAAUC | |
| | CUAACCACGGAACAGGCGGUCGCGAACCAGUGACUGGCUUGUCGUAACGCGCAAGUCUGUGGCGGAA | |
| | CCGACUACUUUGGGUGUCCGUGUUUCCUGUUAUUUUUAUCAUGGCUGCUUAUGGUGACAAUCAGAGA | |
| | UUGUUAUCAUAAAGCGAAUUGGAUUGGCCAUCCGGUGAGUGUUGUGUCAGGUAUACAACUGUUUGUU | |
| | GGAACCACUGUGUUAGCUUUACUUCUCAUUUAACCAAUUAAUCAAAAACAAUACGAGGAUAAAACAA | |
| | CAAUACUACA | |
| 341 | UUAAAACAGCUCUGGGGUUGUUCCCACCCCAGAGGCCCACGUGGCGGCUAGUACACUGGUAUCACGG | poliovirus3 |
| | UACCUUUGUACGCCUGUUUUAUACUCCCUCCCCCGCAACUUAGAAGCAUACAAUUCAAGCUCAAUAG | |
| | GAGGGGGUGCAAGCCAGCGCCUCCGUGGGCAAGCACUACUGUUUCCCCGGUGAGGCCGCAUAGACUG | |
| | UUCCCACGGUUGAAAGUGUCCGAUCCGUUAUCCGCUCAUGUACUUCGAGAAGCCUAGUAUCGCUCUG | |
| | GAAUCUUCGACGCGUUGCGCUCAGCACUCAACCCCGGAGUGUAGCUUGGGCCGAUGAGUCUGGACAG | |
| | UCCCCACUGGCGACAGUGGUCCAGGCUGCGCUGGCGGCCCACCUGUGGCCCAAAGCCACGGGACGCU | |
| | AGUUGUGAACAGGGUGUGAAGAGCCUAUUGAGCUACAUGAGAGUCCUCCGGCCCCUGAAUGCGGCUA | |
| | AUUCUAACCAUGGAGCAGGCAGCUGCAACCCAGCAGCCAGCCUGUCGUAACGCGCAAGUCCGUGGCG | |
| | GAACCGACUACUUUGGGUGUCCGUGUUUCCUUUUAUUCUUGAAUGGCUGCUUAUGGUGACAAUCAUA | |
| | GAUUGUUAUCAUAAAGCGAGUUGGAUUGGCCAUCCAGUGUGAAUCAGAUUAAUUACUCCCUUGUUUG | |
| | UUGGAUCCACUCCCGAAACGUUUUACUCCUUAACUUAUUGAAAUUGUUUGAAGACAGGAUUUCAGUG | |
| | UCACA | |
| 342 | UUAAAACAGCUCUGGGGUUGUUCCCACCCCAGAGGCCCACGUGGCGGCCAGUACUCUGGUAUUGCGG | coxsackievirA1 |
| | UACCUUUGUACGCCUGUUUUAUAUCCCCUCCCCAAGUAACUUUAGAAGCAAUUCACAAAGUUCAAUA | |
| | GAGGGGGUACAAACCAGUACCACCAUGAACAAGCACUUCUGUUUCCCCGGUGAAGCUGUAUAGACUG | |
| | UUCCCACGGUCGAAAAUGGCAGAUCCGUUAUCCGCUCACGUACUUCGAGAAGCCUAGUAUCAUCUUG | |
| | GAAUCUUCGACGCGUUGCGCUCAGCACUCUACCCCGAGUGUAGCUUAGGCCGAUGAGUCUGGGCAUA | |
| | CCCCACCGGCGACGGUGGCCCAGGCUGCGUUGGCGGCCUACCCAUGGCUAAUGUCAUGGGACGCUAG | |
| | UUGUGAACAAGGUGUGAAGAGCCUAUUGAGCUACUUGAGAGUCCUCCGGCCCCUGAAUGCGGCUAAU | |
| | CCUAACCAUGGAGCAAGUGCCCACAAGCCAGUGGGUGGCUUGUCGUAAUGCGCAAGUCUAUGGCGGA | |
| | ACCGACUACUUUGGGUGUCCGUGUUUCCUUUUAUUCUUAUUAUGGCUGCUUAUGGUGACAACCUAAG | |
| | AUUGUUGUCAUAUAGCUUUUGGAUUGGCCAUCCGGUGAUCUUUGAGAUUAAUAUACCUUCCUUAUUA | |
| | CUUCAUAGUACUCAUUUAUUCAUUACACUACUGUAGUUACA | |
| 343 | UUAAAACAGCUGUGGGGUUGUUCCCACCCCAGAGGCCCACGUGGCGGCUAGUACUCCGGUAUCACGG | coxsackievirA11 |
| | UACCCUUGUACGCCUGUUUUAUACUCCCUCCCCCGUAACUUAGAAGCAAUCAAAUCAAGUUCAAUAG | |
| | GAGGGGGUACAAACCAGUACCACCACGAACGAGCACUUCUGUUUCCCCGGUGAAGUUGCAUAGACUG | |
| | UUACCACGGUUGAAAGCGACCGAUCCGUUAUCCGCUCAUGUACUUCGAGAAGCCUAGUACCUCCUUG | |
| | GAAUCUUCGAUGCGUUGCGCUCAGCACUCAACCCCGGAGUGUAGCUUAGGCUGAUGAGUCUGGACAG | |
| | CCCUCACCGGCGACGGUGGUCCAGGCUGCGUUGGCGGCCUACCUGUGGCCCAAAGCCACAGGACGCU | |
| | AGUUGUGAACAAGGUGUGAAGAGCCUAUUGAGCUACAUGAGAGUCCUCCGGCCCCUGAAUGCGGCUA | |
| | AUCCUAACCACGGAGCAGGCGAUCGCAAACCAGCAAUUAGCCUGUCGUAACGCGAAAGUCUGUGGCG | |
| | GAACCGACUACUUUGGGUGACCGUGUUUCUCUUAUUUUUAUUCAUGGCUGCUUAUGGUGACAAUCAU | |
| | AGAUUGUUAUCAUAAAGCGAGUUGGAUUGGCCAUCCGGUAAAAGUCAAGUAUAUCAUUUACUUGUUU | |
| | GUAGGAUUUAUUCCUUUGAACGUCCAUUUACUCAAUCUUAUCAGAGUCACAUUACUAGUAAGAUCUC | |
| | ACUAUCACA | |
| 344 | UUAAAACAGCUCUCGGGUUGUUCCCCCCCCAGAGGCCCACGUGGCGGCCAGUACUCCGGUAUUACGG | coxsackievirA13 |
| | UACCCUUGUACGCCUGUUUUGUAUCCCCUUCCCCCGCAACUUAGAAGUUAUAAACCAAGUUCAAUAG | |
| | AAGGGGGUACAAGCCAGUACCACCACGAACAAGCACUUCUGUUUCCCCGGUGAUGUUGUAUAGACUG | |
| | CUAACGCGGUUGAAAACAGCUGAUCCGUUAUCCGCUUGUGUACUUCGAGAAGCCUAGUAUCAUCUUG | |
| | GAAUCUUCGAUGCGUUGCGUUCAGCGCUCUACCCCAGAGCGUAGCUUAGGCUGAUGAGUCUGGGCAU | |
| | UCCCCACCGGCGACGGUGGCCCAGGCUGCGUUGGCGGCCUACCUAUGGCUAACGCCAUAGGACGCUA | |
| | GUUGUGAACAAGGUGUGAAGAGCCUAUUGAGCUACACAAGAGUCCUCCGGCCCCUGAAUGCGGCUAA | |
| | UCCUAACCAUGGAGCAAGUGAUCACAAUCCAGUGAUAUUCUUGUCGUAACGCGCAAGUCCGUGGCGG | |
| | AACCGACUACUUUGGGUGUCCGUGUUUCCUUUUACUUUUAUCAUGGCUGCUUAUGGUGACAAUCAUU | |
| | GAUUGUUAUCAUAAAGCGAAUUGGAUUGGCCAUCCGGUGAAAGUGAGACUUAUCAUCUACUUGUUUG | |
| | UUGGAUUCACUCCAUUGAACACAUUCACUCCCAGUAUCAUUUCAAUAGUAAUCAGAAUUAGACAGUU | |
| | GUAUCACA | |
| 345 | UUAAAACAGCUCUGGGGUUGUUCCCACCCCAGAGGCCCACGUGGCGGCCAGUACUCCGGUAUUGCGG | coxsackievirA13; |
| | UACCCUUGUACGCCUGUUUUACACUCCCUUCCCCGUAACUUAGACGCAAUAAACCAAGUUCACUAGG | coxsackievirusA18 |
| | AGGGGUGCAAACCAGCACCACCACGAACAAGCACUUCUGUUUCCCCGGUGACAUUGUAUAGACUGUA | |
| | UCCACGGUUGAAAACGAUUGAUCCGUUAUCCGCUCUUGUACUUCGAGAAGCCUAGUAUCAUCUUGGA | |
| | AUCUUCGACGCGUUGCGCUCAGCAUUCAACCCCAGAAUGUAGCUUAGGUCGAUGAGUCUGGACAUUC | |
| | CUCACCGGUGACGGUGGUCCAGGCUGCGUUGGCGGCCUACCUGUGACCCAAAGUCACAGGACGCUAG | |
| | UUGUGAACAAGGUGUGAAGAGCCUAUUGAGCUACAAGAGAGUCCUCCGGCCCCUGAAUGCGGCUAAU | |
| | CCUAACCACGGAGCAAGUGCUCACGAACCAGUGAGUGGCUUGUCGUAACGAGCAAUUCUGUGGCGGA | |
| | ACCGACUACUUUGGGUGUCCGUGUUUCCUUUUUAACUUUAAAUGGCUGCUUAUGGUGACAAUCAUUG | |
| | AUUGUUAUCAUAAGCCGAAUUGGAUUGGCCAUCCGGUGAAAAUCAAGUUGAUCAUUUAUUUGUUUGU | |
| | UGGAUUCACUCCAUUAACUCAUUUUUCAAUUGACCUAAUACGUAUUGUAUUAUUAGUUAGAAACAUA | |
| | CAUCACA | |
| 346 | UUAAAACAGCUCUGGGGUUGUUCCCACCCCAGAGGCCCACGUGGCGGCUAGUACUCCGGUAUUGCGG | coxsackievirA17 |
| | UACCCUUGUACGCCUGUUUUAUACUCCCUUCCCCCGUAACUUAGAAGCAAUCAAACAAAGUUCAAUA | |
| | GAAGGGGGUACAAACCAGUACCACCACGAACAAGCACUUCUGUUUCCCCGGUGAAGUUGUAUAGACU | |
| | GUACCCACGGUUAAAAGCAAUUGAUCCGUUACCCGCUCUUGUACUUCGAGAAGCCUAGUACUACCUU | |
| | GGAAUCUUCGAUGCGUUGCGCUCAACACUCAACCCCAGAGUGUAGCUUGGGCUGAUGAGUCUGGACG | |
| | UUCCCCACCGGCGACGGUGGUCCAGGCUGCGUUGGCGGCCUACCUGUGGCCCAAAGCCACAGGACGC | |
| | UAGUUGUGAACAAGGUGCGAAGAGCCUAUUGAGCUACAAGAGAGUCCUCCGGCCCCUGAAUGCGGCU | |
| | AAUCCCAACCACGGAGCAGGUAGUUGCAAGCCAGCAAUCGACCUGUCGUAACGCGUAAGUCUGUGGC | |
| | GGAACCGACUACUUUGGGUGUCCGUGUUUCCUUUUAAUUUUAAACUGGCUGCUUAUGGUGACAAUCA | |
| | UUGAUUGUUAUCAUAAAGCGAAUUGGAUUGGCCAUCCGGUGAGAAUCAGAUCCAUUAUCUACUUGUU | |
| | CAUUGGAUUUACUCCAUUGACAACAUACACAUACGACCUUAUUAGAGUCAAUUUACUAUUGAGACAU | |
| | CUCCACCAUA | |
| 347 | UUAAAACAGCUCUGGGGUUGUUCCCACCCCAGAGGCCCACGUGGCGGCUAGUACUCUGGUAUUACGG | coxsackievirA19 |
| | UACCUUUGUACGCCUGUUUUACACUCCCUUCCCUACGCAACUUUAGAAGUUUAACAUAAAGUUCAAU | |
| | AGAGGGGGUACAAACCAGCACCACCACGAACAAGCACUUCUGUUUCCCCGGUGAAACUGCAUAGACU | |
| | GUAACCACGGUUGAAAGCGGUUGAUCCGUUAUCCGCUUGAGUACUUCGAGAAGCCUAGUACCACCUU | |
| | GGAAUCUCCGAUGCGUUGCGCUCCACACUCUACCCCGAGUGUAGCUUAGGCCGAUGAGUCUGGGCGU | |
| | UCCCCACCGGCGACGGUGGCCCAGGCUGCGUUGGCGGCCUACCCAUGGUUAACGCCAUGGGACGCUA | |
| | GUUGUGAACAAGGUGUGAAGAGCCUAUUGAGCUACUCAAGAGUCCUCCGGCCCCUGAAUGCGGCUAA | |
| | UCCCAACCACGGAGCGAGUGCUCACAAUCCAGUGAGUGGCUUGUCGUAACGCGCAAGUCUGUGGCGG | |
| | AACCGACUACUUUGGGUGUCCGUGUUUCCUUUUAUUUUUACUAUGGCUGCUUAUGGUGACAAUCUAG | |
| | GAUUGUUAUCAUAUAGCUUUUGGAUUGGCCAUCCGGUGACUUCUAACGUUCAAGUAGUUUACUCGCU | |
| | UAAUUUUCUAAACACAUUCUCAGUUGUUGAAUUAUAAACUUCACA | |
| 348 | UUAAAACAGCUCUGGGGUUGUACCCACCCCAGAGGCCCACGUGGCGGCUAGUACUCCGGUAUUACGG | coxsackievirA20 |
| | UACCCUUGUACGCCUGUUUUAUACUCCCUCCCUCGUAACUUAGAAGCACAAAACCAAGUUCAAUAGA | |
| | AGGGGGUGCAAACCAGUACCACCACGAACAAGCAUUUCUGUUUCCCCGGUGAUGUUGUAUAGACUGC | |
| | CCGCGCGGUUGAAAGCAACGGAUCCGUUACCCGCUCAAGUACUUCGAGAAGCCUAGUAUUACCUUGG | |
| | AAUCUUCGAUGCGUUGCGUUCAGCACUCGACCCUGGAGUGUAGCUUAGGCUGAUGAGUCUGGACGUC | |
| | CCUCACCGGUGACGGUGGUCCAGGCUGCGUUGGCGGCCUACCUAUGGCUAACGCCAUAGGACGCUAG | |
| | UUGUGAACAAGGUGUGAAGAGCCUAUUGAGCUACUUGAGAGUCCUCCGGCCCCUGAAUGCGGCUAAU | |
| | CCUAACCAUGGAGCAGGCGGUCACAGACCAGUGACUAGCUUGUCGUAAUGCGCAAGUCUAUGGCGGA | |
| | ACCGACUACUUUGGGUGUCCGUGUUUCCUUUUAUUUUUAUUAUGGCUGCUUAUGGUGACAAUCAUUG | |
| | AUUGUUAUCAUAAAGCGAAUUGGAUUGGCCAUCCGGUGAAAGCGAGACUUACUAUUUACUUACUUGU | |
| | UGGACUUACCACACUUAAUACAUUUAUUCUAGGUGUCACUUGUAUAGCAAUUAGAAUCAAACAGUUG | |
| | CAUCAUA | |
| 349 | UUAAAACAGCUCUGGGGUUGUUCCCACCCCAGAGGCCCACGUGGCGGCUAGUACUCUGGUAUUACGG | coxsackievirA21 |
| | UACCUUUGUACGCCUGUUUUGUAUCCCUUCCCCCGUAACUUUAGAAGCUUAUCAAAAGUUCAAUAGC | |
| | AGGGGUACAAACCAGUACCUCUACGAACAAGCACUUCUGUUUCCCCGGUGAUAUCACAUAGACUGUA | |
| | CCCACGGUCAAAAGUGAUUGAUCCGUUAUCCGCUUGAGUACUUCGAGAAGCCUAGUAUCACCUUGGA | |
| | AUCUUCGAUGCGUUGCGCUCAACACUCUGCCCCGAGUGUAGCUUAGGCUGAUGAGUCUGGGCACUCC | |
| | CCACCGGCGACGGUGGCCCAGGCUGCGUUGGCGGCCUACCCAUGGCUGAUGCCGUGGGACGCUAGUU | |
| | GUGAACAAGGUGUGAAGAGCCUAUUGAGCUACUCAAGAGUCCUCCGGCCCCUGAAUGCGGCUAAUCC | |
| | UAACCACGGAGCAACCGCUCACAACCCAGUGAGUAGGUUGUCGUAAUGCGUAAGUCUGUGGCGGAAC | |
| | CGACUACUUUGGGUGUCCGUGUUUCCCUUUAUAUUCAUACUGGCUGCUUAUGGUGACAAUUUACAAA | |
| | UUGUUACCAUAUAGCUAUUGGAUUGGCCACCCAGUAUUGUGCAAUAUAUUUGAGUGUUUCUUUCAUA | |
| | AGCCUUAUUAACAUCACAUUUUUAAUCACAAUAAACAGUGCAA | |
| 350 | UUAAAACAGCCUGGGGUUGUUCCCACCCCAGAGGCCCACGUGGCGGCCAGUACUCUGGUAUUACGGU | coxsackievirA22 |
| | ACCUUUGUACGCCUGUUUUAUAUCCCCUCCCCUGAGUAACUUUAGAAGCAAUUCAAAAGGUUCAAUA | |
| | GAGGGGGUACAAUCCAGUACCACCAUGAACAAGCACUUCUGUUUCCCCGGUGAGGCUACAUAAACUG | |
| | UACCCACGGUUGAAAGUGGCUAAUCCGUUAUCCGCUCAAGUACUUCGAGAAGCCUAGUAAUACCUUG | |
| | GAAUCUUCGACGCGUUGCGCUCAGCACUCUAACCCGGGUGUAGCUUAGGUCGAUGAGUCUGGGCAUA | |
| | CCCCACCGGUGACGGUGGCCCAGGCUGCGUUGGCGGCCUACCCAUGGCUAACGCCAUGGGACGCUAG | |
| | UUGUGAACAAGGUGUGAAGAGCCUAUUGAGCUACUUGAGAGUCCUCCGGCCCCUGAAUGCGGCUAAU | |
| | CCUAACCACGGAGCAAGUGCUCUCAAACCAGAGAGUGGCUUGUCGUAACGCGUAAGUCUGUGGCGGA | |
| | ACCGACUACUUUGGGUGUCCGUGUUUCCUUUUAUUUUUAUCAUGGCUGCUUAUGGUGACAAUCUAAG | |
| | AUUGUUAUCAUAUAGCUUUUGGAUUGGCCAUCCGGUUGUUGUUUAGAGCGUUUCAUCUGUGCUUUGU | |
| | UUGUUUGACCAGAUCAAUAUAAACACUACAUCCUAUUUGUUCAUA | |
| 351 | UUAAAACAGCUCUGGGGUUGUUCCCACCCCAGAGGCCCACGUGGCGGCCAGUACUCCGGUAUUGCGG | coxsackievirA24 |
| | UACCCUUGUACGCCUGUUUUACACUCCCUUCCCCGGUAACUUAGAAGUAAACAAACCAAGUUCAAUA | |
| | GAAGGGGGUACAAACCAGUACCACCACGAACAAGCACUUCUGUUUCCCCGGUGACGUUGUAUAGACU | |
| | GUACCCACGGUUGAAAACAACUGAUCCGUUAUCCGCUCUUGUACUUCGAGAAGCCCAGUAUCACCUU | |
| | GGAAUCUUCGAUGCGUUGCGCUCAGCACUCAACCCCAGAGUGUAGCUUAGGUCGAUGAGUCUGGACA | |
| | AUCCUCACCGGCGACGGUGGUCCAGGCUGCGUUGGCGGCCUACCUGUGGCCCAAAGCCACAGGACGC | |
| | UAGUUGUGAACAAGGUGUGAAGAGCCUAUUGAGCUACGAGAGAGUCCUCCGGCCCCUGAAUGCGGCU | |
| | AAUCCCAACCACGGAGCAGGUGAUCACAAGCCAGUGAUUAGCCUGUCGUAACGCGCAAGUCUGUGGC | |
| | GGAACCGACUACUUUGGGUGUCCGUGUUUCCUUUAUUUUAUUAUGGCUGCUUAUGGUGACAAUCACA | |
| | GAUUGUUAUCAUAAAGCGACUUGGAUUGGCCAUCCAGUGAAAAUAAAACACAUCAUUUACCUGAUUG | |
| | UUGGAUUUACUCCGCUGACUAUCUACUCACCAGGUCUUAUUAAGGUUAUUCUGUUGAUUAGGUCACA | |
| | AUUUCACA | |
| 352 | UUAAAACAGCUCUGGGGUUGUUCCCACCCCAGAGGCCCACGUGGCGGCCAGUACUCCGGUAUCACGG | enterovirusC96 |
| | UACCCUUGUACGCCUGUUUUAUACCCCCUUCCCCGCAACUUAGAAGCUUUAAAUUCAAGUUCAAUAG | |
| | AAGGUGGUACAAACCAGUAUCGCCACGAACAAGCACUUCUGUUGCCCCGGUGAAAUUGCAUAAGCUG | |
| | UCCCCACGGCUGAAGGCGAUUGAUCCGUUACCCGCUCUUGUACUUCGAGAAGCCUAGUACCACCUUG | |
| | GAAUUUUCGAUGCGUUGCGCUCAGCACUCAACCCCAGAGUGUAGCUUAGGCUGAUGAGUCUGGACAU | |
| | ACCCCACCGGCGACGGUGGUCCAGGCUGCGUUGGCGGCCUACCUGUGGCCCAAAGCCACAGGACGCU | |
| | AGUUAUGAACAAGGUGUGAAGAGCCUAUUGAGCUACAAGAGAGUCCUCCGGCCCCUGAAUGCGGCUA | |
| | AUCCUAACCACGGAUCAGGCGAUUGCAACCCAGCAAUUAGCCUGUCGUAAUGCGCAAGUCUGUGGCG | |
| | GAACCGACUACUUUGGGUGUCCGUGUUUCUUUUUAUUCUUACAUUGGCUGCUUAUGGUGACAAUCUU | |
| | AGAUUGUUAUCAUAAAGCAAGUUGGAUUGGCCAUCCGGUGAAAGUCAAAGUUACUAUCUAUUUAUCU | |
| | GUUGGAUUUUACCCUAAGACCACUCAUUUACCUGACUUAAUUAGAAUAAUUAUACUACUUGGAAAUU | |
| | ACUAUCACA | |
| 353 | UUAAAACAGCUCUGGGGUUGUUCCCACCCCAGAGGCCCACGUGGCGGCUAGUACUCUGGUAUCACGG | enterovirusC99 |
| | UACCUUUGUACGCCUGUUUUAUAUCCCUUCCCCCGUAACUUAGAAGCUUUGUACCAACAAGUUCAAU | |
| | AGAAGGGGUACAAACCAGUACCACCACGAACAAGCACUUCUGUUUCCCCGGUGCGAUGACAUAGACU | |
| | GUACCCACGGUCAAAAGUCACCAAUCCGUUACCCGCUUUGGUACUUCGAGACGCCUAGUAUUGCCUU | |
| | GGAAUUCUCGAUGCGUUGCGCUCAACACUCGACCCUGGAGUGUAGCUUAGGCUGAUGAGUCUGGGCA | |
| | UUCCCCACCGGUGACGGUGGCCCAGGCUGCGUUGGCGGCCUACCCAUAGCUAUUGCUAUGGGAUGCU | |
| | AGAGUUGAACAAGGUGUGAAGAGCCAAUUGAGCUACUAAAGAGUCCUCCGGCCCCUGAAUGCGGCUA | |
| | AUCCCAACCACGGAUCAAGUGCGUGCAACCCAGCACGUAGCUUGUCGUAAUGCGCAAGUCUGUGGCG | |
| | GAACCGACUACUUUGGGUGUCCGUGUUUCCUUUUAUUUUUAUCAUGGCUGCUUAUGGUGACAAUCUU | |
| | GGAUUGUUAUCAUAUAGCGAUUGGAUUGGCCAUCCGGUGAGAAUAAAAGAUAUUGUUUAUCUGUUUG | |
| | UGGCUUUUACUCCAUUAAACAAAAACACACUUUCCUUAAUUAAAGUGGUACUAUUGAUCAGAUCAGC | |
| | AUACCACA | |
| 354 | GGUAACCUUGUACGCCUGUUUUAUAAUCCCCUUCCCCCGUAACAGUAGAAGCACAAAAGCAACCGCU | enterovirusC104 |
| | CAAUAGAUGCCAUACCUCCAGUGUGGUUAUGAGCAAGCACUUCUGUCUCCCCGGUUGCGCCCUAUAU | |
| | GCUGUGCGAACGGCAAAAAGGGGCAAUAUCGUUACCCGCUUGACUACUACGGGAAACCUAGUAUUAU | |
| | CUUCGAUUGAUUUGAGAGUUGCGUUCAGCGCCCAACCCCCGGCGUAGCUCAGGCUGAUGAGGCUCCG | |
| | AACACCCCACAGGCGACUGUGUCAGAGCCUGCGUUGGCGGCCUGCCCAUACCCUGCGGUAUGGGACG | |
| | CUCACUUCAUGACAUGGUAUGAAGAGUCUACUGUGCUAGUCAAGAUUCCUCCGGCCCCUGAAUGUGG | |
| | AUAAUCCUAACCACUGAUCCUACGUUCGCAAUCCAGCGUUUGGUAGGUCGUAAUGCGCAAGUCGGUG | |
| | GCGGAACCAACUACUUUGGGUGUCCGUGUUUCCCUUACUUCCUACUGAAUGCUUAUGGUGACAAUUG | |
| | UUUUGAUCAGUUGUUGCCAUUACUUGCGUUCAGUGUGUAAUUACAAAAACAAGAUCAUCAGUUGUUA | |
| | CUAUCCCUCACGUUCAGUGCGCAAUUACAAAAACAAGAUUUUCUGUCAUA | |
| 355 | CCCCUAGGAUCCACUGGAUGUUAGUACACUGGUAUCGCGGUACCUUUGUACGCCUGUUUUAUAUCCC | enterovirusC105 |
| | CUUCCCCGCAACUUUAGAAGUAUCAAAAGUACCGCUCAAUAGCCGCCACGCUCCCAGUGUGGUUUCG | |
| | AGCAAGCACUUCUGUUUUCCCGGUUGCGUCCCAUAUGCUGUGCAAACGGCAAAAAGGGACAAUAUCG | |
| | UUACCCGCUUGUAUACUACGGGAAACCUAGUACCGCCAUUGAUUGUAUUGAGGGUUGCGCUCAUCAC | |
| | CUUUCCCGGUGUAGCUCAGGCUGAUGAGGCUCAGAAUCCCCCACAGGUAACUGUGUCUGAGCCUGCG | |
| | UUGGCGGCCUGCCCUCGCCUUAUGGCGUGGGACGCUCACUACAUGACAUGGUGCGAAGAGUCUACUG | |
| | UGCUAUACAAGAGUCCUCCGGCCCCUGAAUGUGGAUAAUCCUAACCACUGAUCCCACGUACGCAAAC | |
| | CAGCGUGUAGUGGGUCGUAACGCGCAAGUCGGUGGCGGAACCGACUACUUUGGGUGACCGUGUUUCC | |
| | UUUAUUACCUAUUGAAUGUUUAUGGUGACAAUUGUUUGAUUCAGUUGUUACCAUUUGCUACAUUCAA | |
| | UAACCUAGUGUCAAACCGAUUGAAUUGCUACA | |
| 356 | ACUCUGGUAUUGCGGUACCUUUGUACGCCUGUUUUAUAUCCCUUCCCCAUGUAACUUUAGAAGCAAU | enterovirusC113 |
| | UCACAAGGUUCAAUAGAGGGGGUACAAACCAGUACCACCACGAACAAGCACUUCUGUUUCCCCGGUG | |
| | AAGCUGUAUAGACUGUCCCCACGGUCAAAAACGGCAGAUCCGUUAUCCGCUUAUGUACUUCGAGAAG | |
| | CCUAGUAUCAUCUUGGAAUCUUCGAUGCGUUGCGUUCAGCACUCUAUCCCGAGUGUAGCUUAGGUCG | |
| | AUGAGUCUGGGCAUUCCCCACCGGCGACGGUGGCCCAGGCUGCGUUGGCGGCCUACCCAUGGCUAAC | |
| | GCCAUGGGACGCUAGUUGUGAACAAGGUGUGAAGAGCCUAUUGAGCUACAUGAGAGUCCUCCGGCCC | |
| | CUGAAUGCGGCUAAUCCCAACCACGGAGCAAGUACUCACAACCCAGUGGGCAGCUUGUCGUAACGCG | |
| | UAAGUCUGUGGCGGAACCGACUACUUUGGGUGUCCGUGUUUCCUUUUUAUUUUUGAUAUGGCUGCUU | |
| | AUGGUGACAUCUAAGAUUGUUAUCAUAUAGCUUUUGGAUUGGCCAUCCGGUGAUUUUUGAGACUAAA | |
| | ACGCUUGUUUGCCUUAUCACACAAUUUAGUAUUUUGUUAUAUUCAUACGGUCAUA | |
| 357 | UUCCCACUCCAGAGGCCCACGUGGCGGCUAGUACUCUGGUAUUGCGGUACCUUUGUACGCCUGUUUU | enterovirusC116 |
| | AUACCCCCUCCCCAAGUAACUUUAGAAGCAAUUCACAAAGUUCAAUAGAGGGGGUACAAACCAGUAC | |
| | CACCACGAACAAGCACUUCUGUUUCCCCGGUGAAGCUACAUAGACUGUCCCCACGGUCGAAGGUGGC | |
| | AGAUCCGUUAUCCGCUCAAGUACUUCGAGAAGCCUAGUACCACCUUGGAAUCUUCGAUGCGUUGCGC | |
| | UCAGCACUCUACCCCGAGUGUAGCUUAGGUCGAUGAGUCUGGGCAUGCCCCACCGGUGACGGUGGCC | |
| | CAGGCUGCGUUGGCGGCCUACCCGUGGCUAACGCCAUGGGACGCUAGUUGUGAACAAGGUGUGAAGA | |
| | GCCUAUUGAGCUACUUGAGAGUCCUCCGGCCCCUGAAUGCGGCUAAUCCUAACCACGGAGCAAGUGC | |
| | UCACAAACCAGUGAGUGGCUUGUCGUAACGCGUAAGUCUGUGGCGGAACCGACUACUUUGGGUGUCC | |
| | GUGUUUCCUUUUAUUUUCAUUAUGGCUGCUUAUGGUGACAAUCUAAGAUUGUUAUCAUAUAGCUUUU | |
| | GGAUUGGCCAUCCGGUGAUUUGAAAGAGAUAUAUUUACCUUAUUGUUCUACCAUAUCUAAUUACUCA | |
| | UCACAGCCACAUAAUCACA | |
| 358 | UUUAAAACAGCUUGAGGGUUGUUCCCACCCUCAAGGCCUACUGGGCGCUAGUACACCGGUAUCUCGG | enterovirusC117 |
| | UACCCUUGUACGCCUGUUUUAUCUUCCCUCCCCAAAAGUAACUUUAGAAGCAAGUAAACUCACAGCU | |
| | CAAUAGCCGUCGCACCGCCAGUGCGACUAUGAGCAAGCACUCCUGUUUCCCCGGUUGCGUCCUAUAU | |
| | GCUGUGGAAACGGCAGAAGGGGACAAUAUCGUUACCCGCUUAUCUACUACGGGAAGCCUAGUAACAC | |
| | CAUUGAACACUUUGAGGGUUGCGUUCAGCGCCAAACCCGGCGUAGCUCAGGCCGAUGGGGCUUCGGA | |
| | AUCCCCACGGGUGACCGUGUCGAAGCCCGCGUUGGCGGCCUGCCCACACCAAUGGGUGUGGGACGCU | |
| | CAAUUUGUGACAUGGUGCGAAGAGCCUACUGUGCUAGGUGAGAGUCCUCCGGCCCCUGAAUGUGGCU | |
| | AAUCCCAACCACCGAACCCGUGGUUGCAAUCCAGCAACUAGCGGGCCGUAAUGCGCAAGUCGGUGAC | |
| | GGAACCAACUACUUUGGGUGACCGUGUUUCCUUUAUCUCUUAUAUUGCAAUUUUAUGGUGACAUUUA | |
| | UAGUAUUUAAGUGUUACCAUAUAUUGCGCAAACAUAUUGUAAUAAUACAGAGUUACUACUGCAGAAA | |
| | UC | |
| 359 | UUAAAAAAACUUGGGGGGUUUUUCCCCCCCCGGGGUUCCCCGGGAGUCCGUACCCCGGCAUCCUGGG | enterovirusC118 |
| | GCCCUUGUAAGCCUUUUUUAUAAUUCCCCCCCCCCGCAACUUUAGAAGAAAACGAACUCCGAGCUCA | |
| | GUAGGCGUCACGUUUCCAGCGUGACUACGAGCAAGCACUUCUGUUUCCCCGGUUGCGCCCUAUAUGC | |
| | UGUGUGAACGGCAGAAAGGGGCAAAAUCGUUAUCCGCUUGAUUACUGCGGGACGCCUAGUAUCACCA | |
| | UGGAUUGACUUGAGGGUUGCGCUCAGCACACCCCCAGUGUAGCUUGGGCCGAUGAGGCUCCAAAUUC | |
| | CCCACGGGCGACCGUGUUGGAGCCUGCGUUGGCGGCCUACCCGCGCCUCACGGCGUGGGACGCUCAU | |
| | UUCAUGACAUGGUGCGAAGAGUCUACUGCGCUAGUCAAGAUUCCUCCGGCCCCUGAAUGUGGCUAAU | |
| | CCUAACCACUGAUCCUGUGAGCACACACCAGUGCCUAGCAGGUCGUAAUGCGCAAGUCGGUGGCGGA | |
| | ACCAACUACUUUGGGUGUCCGUGUUUCCUUUAUUUCCUCAUUGAAUGCUUAUGGUGACAAUUGUUGA | |
| | GUACAGUUGUUACCAUUACUGCAUUCUGCAAAUAGUGAAUAAACUACGCAACAAUAUAACACA | |
| 360 | UUAAAACAGCUCUGGGGUUGUUCCCACUCAAGGGCCCACGUGGCGGCUAGUACUCUGGUAUCUCGGU | enterovirusD68 |
| | ACCUUUGUACGCCUGUUUUAAUUCCCUCCCCAACGUAACUUAGAAGCUUUUAAACCAAAGCUCAAUA | |
| | GGUGGAGCGCAAACCAGCGCUCUUAUGAGCAAGCACUUCUGUCUCCCCGGUGUGGUUGUAUAGACUG | |
| | UCCCCACGGUUGAAAACAACUUAUCCGUUAACCGCUAUAGUACUUCGAGAAACCUAGUAUUGCCUUC | |
| | GGAGUGUUGAUGCGUUGCGCUCAGCACACUAACCCGUGUGUAGCUUGGGUCGAUGAGUCUGGACGUA | |
| | CCCCACUGGCGACAGUGGUCCAGGCUGCGUUGGCGGCCUACUCAUGGUGAAAACCAUGAGACGCUAG | |
| | ACAUGAACAAGGUGUGAAGAGUCUAUUGAGCUGCUAUAGAGUCCUCCGGCCCCUGAAUGCGGCUAAU | |
| | CCUAACCAUGGAGCAAGUGCUCACAAACCAGUGAGUUACUUGUCGUAACGCGCAAGUCCGUGGCGGA | |
| | ACCGACUACUUUGGGUGUCCGUGUUUCACUUUUUACUUUUAUGACUGCUAAUGGUGACAAUUUAAUA | |
| | UUGUUACCAUUUGGCUUGUCGAAUUGAUCACAUAAGAUCUAUAGUUUUGUUCACUGAUUUGCUUUGA | |
| | AAUAAUCUCACCUCAAAACCUCCAGUACAUAACAUUUAAAGAGUUUAAACUUAUUUAUAACA | |
| 361 | UUAAAACAGCUCUGGGGUUGUUCCCACCCCAGAGGCCCACGUGGCGGCUAGUACUCCGGUACCCCGG | enterovirusD70 |
| | UACCCUUGUACGCCUGUUUUAUACUCCCUUUCCCAAGUAACUUUAGAAGAAAUAAACUAAUGUUCAA | |
| | CAGGAGGGGGUACAAACCAGUACCACCACGAACACACACUUCUGUUUCCCCGGUGAAGUUGCAUAGA | |
| | CUGUACCCACGGUUGAAAGCGAUGAAUCCGUUACCCGCUUAGGUACUUCGAGAAGCCUAGUAUCAUC | |
| | UUGGAAUCUUCGAUGCGUUGCGAUCAGCACUCUACCCCGAGUGUAGCUUGGGUCGAUGAGUCUGGAC | |
| | ACCCCACACCGGCGACGUGGUCCAGGCUGCGUUGGCGGCCUACCCAUGGCUAGCACCAUGGGACGCU | |
| | AGUUGUGAACAAGGUGCGAAGAGCCUAUUGAGCUACCUGAGAGUCCUCCGGCCCCUGAAUGCGGCUA | |
| | AUCCCAACCACGGAGCAAAUGCUCACAAUCCAGUGAGUGGUUUGUCGUAAUGCGCAAGUCUGUGGCG | |
| | GAACCGACUACUUUGGGUGUCCGUGUUUCCUUUUAUUUUUAUUAUGGCUGCUUAUGGUGACAAUCUG | |
| | AGAUUGUUAUCAUAUAGCUAUUGGAUUAGCCAUCCGGUGAUAUCUUGAAAUUUUGCCAUAACUUUUU | |
| | CACAAAUCCUACAACAUUACACUACACUUUCUCUUGAAUAAUUGAGACAACUCAUA | |
| 362 | UUUCCCCGGUGAAGUUACAUAGAUUGUGCCCACGGUGAAAGUGACUGAUCCGUUACCCGCUUUAGUA | enterovirusD111 |
| | CUUCGAGAAGCCUAGUAACAUCUUGGAAUCUUCGAUGCGUUGCGCUCAGCACUCAACCCCAGAGUGU | |
| | AGCUUAGGUCGAUGAGUCUGGACACUCCCCACCGGCGACGGUGGUCCAGGCUGCGUUGGCGGCCUAC | |
| | CUGUGGCCCAAAGCCACAGGACGCUAGUUGUGAACAAGGUGUGAAGAGCCUAUUGAGCUACUAAAGA | |
| | GUCCUCCGGCCCCUGAAUGCGGCUAAUCCCAACCACGGAGCAAGUGCUCACAAUCCAGUGAGUGGCU | |
| | UGUCGUAACGAGCAAUUCUGUGGCGGAACCAACUACUUUGGGUGUUCGUGGUUCUUUUUUUAUCAUU | |
| | GGCUGCUUAUGGUGACAAUCUA | |
| 363 | UUAAAACAGCCUGGGGGUUGUACCCACCCCUGGGGCCCACGUGGCGCUAGUACUCUGGUUCGUUAGA | enterovirusE1; |
| | ACCUUUGUACGCCUGUUUUCCCCUCCUUAAACAAAUUAAGAUCUCUGCCAAUGUGGGGAGUAGUCCG | bovine- |
| | ACUCCGCACCGAUACGUCGCACCAGUAGACCGGUUCGCUUAGGACCCUUCUACGGAUUGGUAUGAGU | enterovirus1 |
| | UCCCCACCCCGUAACUUAGAAGUACUAGCAAAACCGACCAAUAGGAGCGUGGCACCCAGCUGCGUUA | |
| | AGGUCAAGCACUUCUGUCUCCCCGGCCAGAAAUGGUCGUCACCCGCCCUCUCUACUACGAGAAGCCU | |
| | AUUAACCAUUGAAGGCGAUGAGGAGUUGCGCUCCACCACAACCCCAGUGGUAGCUCUGAGAGAUGGG | |
| | GCUCGCAGUCACCCCCGUGGUAACACGGUUGCUUGCCCGCGUGUGCUCUCGGGUUCGGCCACUUGGC | |
| | CGUUCACUCCAACUCGUUGUAAGUGGCCAAGAGCCUAUUGUGCUAGAGAGGUUUUCCUCCGGAGCCG | |
| | UGAAUGCUGCUAAUCCCAACCUCCGAGCGUGUGCGCACAAUCCAGUGUUGCUACGUCGUAACGCGCA | |
| | AGUUGGAGGCGGAACAGACUACUUUCGGUACUCCGUGUUUCCUUAUUAUUUUAUACAACAAUUUAUG | |
| | GUGACAUUGACUGAUACUAUUGAGUUCGCCCGCUUGCCAUUGAAUAUUGCCUUGUAUUACCUUAUAG | |
| | CAUUUCAAAAAGCCACAGAUCUCACCCUCGAGCUCAUUCACUUUGCAGUUUGUUUGAAUCGCAUACA | |
| | CAAGACAUUUGAAC | |
| 364 | UUAAAACAGCCUGGGGGUUGUACCCACCCCCGGGGCCCACGCGGCGCUAGUACUCUGGUUCGUUUAG | enterovirusE2 |
| | AACCCUUGUACGCCUGUUUUUCCCCCCUUAAAUAAAUUAAGAUCUCUGCCAAUGUGGGGAGUAGUCC | |
| | GACUCCGCACCGAUACGUCGCACCAGCAGACCGGUUCGCCUAGGACCCUUAUGCGGAUUGGUAUGAG | |
| | UCCCCCCCCCGAAACUUAGAAGCACAUUACAAACUGGCCAAUAGGAGCGUGGUAUCCAACCGCGUUG | |
| | CGGUCAAGCACUUCUGUUUCCCCGGUCAGAAAUGAUCGUUACCCGCCCUCCCCACUACGAGAAGCCU | |
| | AUUAACCACUGAAAGUGAUGAUGAGUUGCGCUCAGCCACAACCCCGGUGGUAGCUCUGAGAGAUGGG | |
| | GCUCGCACCACCCCCGUGGUGACACGGUUGCUUGCCCGCGUGUGCUCUCGGGUUCAGUCGCGAGACU | |
| | GUUCACUCUGACUCAUUGUAGGUGGCCAAGAGCCUAUUGUGCUGGGGAGGUUUUCCUCCGGAGCCGU | |
| | GAAUGCUGCUAAUCCCAACCUCCGAGCGUGUGCGCACAAUCCAGUGUUGCUACGUCGUAACGCGUAA | |
| | GUUGGAGGCGGAACAGACUACUUUCGGUACUCCGUGUUUCCUUAAUUUUAUUGUUCAAAUUUAUGGU | |
| | GACAUUGUCUGACAUUUGUGAAUUCGCUCGCCUGCCACUUAAUAUCGCUUUAUAUCAUCUGAUAGCA | |
| | UUCCAUAAGACUACAGAACUCACUUUGGAACUCAUCGACUUUGCUGUUUUCCUGAAUACUGUUCACA | |
| | ACCAAACAAAACAAA | |
| 365 | UUAAAACAGCCUGGGGGUUGUACCCACCCCUGGGGCCCACGUGGCGCCAGUACUCUGGUACGCUAGU | enterovirusE3 |
| | ACCUUUGUACGCCUGUUUUCCCCACCCUUAAAUAAAUUAAGAUUACCACUACUGAGGGGAGUAGUCC | |
| | GACUCCGCUCCGACACUGCCGCACCAGUAUUCCGGUACGCUUAGUACCCUAAUACGGAGUAGAUGGU | |
| | AUCCCCACCCCGCAACUUAGAAGCAUGCAAACAACCCGACCGAUAGGAGCGCGGCAUCCAGUCGCGU | |
| | AUAGGUCAAGCACUUCUGUUUCCCCGGUCCGAAAGGAUCGUUACCCGCCCAACCCACUACGAGAAGC | |
| | CUAGUAACUGGCCAAACAACCGCAAAGUUGCGCUCAGCCACAACCCCAGUGGUAGCUCUGGAAGAUG | |
| | GGGCUCGCACCUCCCCCGUGGUAACACGGUUGCUUGCCCGCGUGUGCUUCCGGGUUCGGCCUAGUGC | |
| | CGUUCACUUUAAAGUUGUGUAACCAGCAAAGAGCCUAUUGUGCUGGGAUGGUUUUCCUCCGGGGCCG | |
| | UGAAUGCUGCUAAUCCCAACCUCCGAGCGUGUGCGCACAAACCAGUGUUGCUACGUCGUAAUGCGUA | |
| | AGUUGGAGGCGGAACAGACUACUUUCGGUACCCCGUGUUUCCUUUUAUUGUUAUUUACAUUUUAUGG | |
| | UGACACUGACUGACAUUUGUGAAUUUGCUCGCUUGCCUCUCAAUAUUGCACUAUAUUACCUGAUUGC | |
| | AUUUCACAACACUAGUAAACUAACCCUUGAACUUAUCGACUUUGCUGUUUACCUAAGUCACUUAAAU | |
| | CACAAGUACAAUA | |
| 366 | CGGAGUAGAUGGUAUUCCCUCUCCCGUAACUUAGAAGUUUGAACUAAACCGACCAAUAGCGGUGGUG | enterovirusE4 |
| | CAACCAGCAUCACAACGGUCAAGCACUUCUGUUUCCCCGGCACGUCAGUGUCGUUACCCGCCCACAC | |
| | UACUACGAGAAGCCUAGUAACUUCCCAAUCGACAGCACAGUUGCGUUCGGCCACAACCCCUGUGGUA | |
| | GCUCUGGAAGAUGGGGCU | |
| 367 | UUUAAAACAGCUCGGGGGUUGUUCCCACUCCCGGGGCCCACGUGGCGCUAGUACUCUGGUACGUUAG | enterovirusE5 |
| | UACCUUGUACGCCUGUUUUCCCCUCCUUAAACAAAUUAAGAUUACCACUACUGAGGGGAGUAGUCCG | |
| | ACUCCGCUCCAGCACUGCUGCACCAGUGAAUUGGUAUGAUUAGUACCUCUUCACGGAGUAGAUGGUA | |
| | UUCCUACCCCGCAACUUAGAAGUUUAUUAACAAACCGACCAAUAGGUGCACAGUAUCCAGCUGUGUA | |
| | ACGGUCAAGCACUUCUGUUUCCCCGGUCCACCAGGAUCGUUACCCGCCCGACCCACUACGAGAAGCC | |
| | UAGUAACUAGCCAAGUGACUGCGAAGUUGCGCUCAACCACAACCCCAGUGGUAGCUCUGGAAGAUGG | |
| | GGCUCGCACCUCCCCCGUGGUAACACGGUUGCCUGCCCGCGUGUGCUUCCGGGUUCGGCCUUGUGCC | |
| | GUUCACUUCUACGUCAUGUAACUAGCCAAGAGCCUAUUGUGCUGGGACGGUUUUCCUCCGGGACCGU | |
| | GAAUGCUGCUAAUCCUAACCUCCGAGCGUGUGCGCACAAUCCAGUGUUGCUACGUCGUAACGCGUAA | |
| | GUUGGAGGCGGAACAGACUACUUUCGGUACCCCGUGUUUCCUCUUUUAUUAUAUUAUUUAAUGGUGA | |
| | CAAUUGCAGAAAUUUGUGAUUUAGCGACUUUACCAUUGCACAUAGCAUUAUAUUGUUUGAUUGCUUU | |
| | CCACAAAACAUCUGAUAUACCCAACUCUUAUAUCGAUCUACUUAUCUUUCUUUGCUUGAAGAAUAAA | |
| | UUAAAGAAAGCACG | |
| 368 | UUCAAACAGCCUGGGGGUUGUACCCACCCCUGGGGCCCACGCGGCGCUAGUACUCUGGUACGUUAGU | enterovirusF2 |
| | ACCUUUGUACGCCUGUUUUCCCCUCCCUUAAAUAAAUUAAGAUUACCACUACUGAGGGGAGUAGUCC | |
| | GACUCCGCUCCGACAAUGUCGCACCAGUGCACUGGUACGCUAGUACCUUUUCACGGAGUAGCUGGUA | |
| | UUCCCUCCCGAAACUUAGAAGCACUUAAAUCAACCGACCAAUAGGCGCGCGGUAGCCAGCCGCGUUA | |
| | CGGUCAAGCACUCCUGUUUCCCCGGUCCACAAGGAUCGUUACCCGCCCGACCCACUGCGAGAAACCU | |
| | AGUAGUUGGCCAAGUGGUAACGAGGUUGCGCUCAGCCACAACCCCAGUGGUAGCUCCGGAAGAUGGG | |
| | GCUCGCACCACCCCCGCGGUAACGCGGUUGCUUGCCCGCGCGUGCUUCCGGGUCCGGUCUCGUACCG | |
| | UUCACCUCAAUACCACGUAACCAGCAAAGAGUCUAUUGCGCUGGGACGGUUUUCCUCCGGGGCCGUG | |
| | AAUGCUGCUAAUCCCAACCUCCGAGCGUGUGCGCACAAUCCAGUGUUGCUACGUCGUAACGCGUAAG | |
| | UUGGAGGCGGAACAGACUACUUUCGGUACCCCGUGUUUCCCUAUCAUUCCAUUUCAUUUUAUGGUGA | |
| | CAAUUGCUGAGAUCUGUGAUUUGGCGACCUUACCACUGAAUAUUGCCUUAUAUUACUUGGUUGCAUU | |
| | CCACAAAACCUCUGACAUACCUGCUUCCUAUAUCGACCUACUUGUUUUCCUCAAUUUAAAGUAUAGA | |
| | CUACAAAUAGCAGA | |
| 369 | UUUAAAACAGCCUGGGGGUUGUACCCACCCCUAGGGCCCACGUGGCGCCAGUACUCUGGUACAUUAG | enterovirusF3 |
| | UACCUUUGUACGCCUGUUUUCCCCUCCCUUAAAUAAAUCAAGAUUACCACUACUGAGGGGAGUAGUC | |
| | CGACUCCGCUCCAGCAAUGCUGCACCAGUGCACUGGUACGCUAGUACCUUUUCACGGAGUAGAUGGU | |
| | AUCCCCACCCCGAAACUUAGAAGCAUGUGAUCAAAUCGACCAAUAGGCGCGCGGUUGCCAGCCGCGU | |
| | UUCGGUCAAGCACUUCUGUUUCCCCGGUCCGCAAGGAUCGUUACCCGCCCGACCCACUGCGAGAAGC | |
| | CCAGUAACUGGCCAAGCGGUAGCGAGGUUGCACUCAGCCACAACCCCAGUGGUAGCUCUGGAAGAUG | |
| | GGGCUCGCAGUUCCCCCGUGGUAACACGGAUGCCUGCCCGCGUGUGCUUCCGGGUUCGGUCUCGUGC | |
| | CGUUCACCUCAAUACCGCGUAACCAGUGAAGAGCCUAUUGCGCUGGGACGGUUUUCCUCCGGGGCCG | |
| | UGAAUGCUGCUAAUCCUAACCUCUGAGCGUGUGCGCACAAUCCAGUGUUGCUACGUCGUAACGCGUA | |
| | AGUCAGGGGCGGAACAGACUACUUUCGGUACCCCGUGUUUCCUUUUAUUUUAUAUUUAUCAUUUUAU | |
| | GGUGACAAUUGCUGAGAUUUGCGAUUUAGCGGUUUUGCCAUUGAACAUUGCUUUGUACUACUUGGUU | |
| | GCAUUUCACAAAACCUCCGACAUACCCAGCUCCUACAUCGACUUGCUUGUUUUUCUUAAUUAAGACA | |
| | UAAACUAGAGAACACAAA | |
| 370 | UUAAAACAGCCUGGGGGUUGUACCCACCCCUAGGGCCCACGUGGCGUUAGUACCCUGGUACGCUAGU | enterovirusF4 |
| | ACCUUUGUACGCCUGUUUUUCCCUCCCUUAAAUAAAUUAAGAUUACCACUACUGAGGGGAGUAGACC | |
| | GACUCCGCUCCAGCACUGCUGCACCAGUGAACUGGUACGCUAGUACCUUUGCACGGAGUAGAUGGUA | |
| | CACCCACCCCGUAACUUAGAAGUACUGAACACAACCGACCAAUAGCGGCGUGACAGCCAGUUACGCA | |
| | ACGGUCAAGCACUUCUGUUUCCCCGGUCCGUAUGGAUCGUUACCCGCCCAAGCUACUACAAGAAGCC | |
| | UAUUAACUACUGAAACUGUUGAAAGGUUGCGCUCAACCACAUCCCCAGUGGUAGCUCCGAGUGAUGG | |
| | GGCUCGCGAUUCCCCCGUGGUAACACGGUCGCUUGCCCGCGCGCGCACUCGGGUCCGGUCUUUGACC | |
| | GUUCACCUUGAGACAACGUAAGUAGCCAAGAGCCUAUUGUGCUGGUUUGGUUAUCCUCCGGAGCCGU | |
| | GAAUGCUGCUAAUCCUAACCCCCAAGCGUGUGCGCACAAACCAGUGUUGCUACGUCGUAACGCGUAA | |
| | GUUGGAGGCGGAACAGACUACUUUCGGUACUCCGUGUUUCCUUUAUCUCAUUUUUAAAAACUUUAUG | |
| | GUGACAAUUGUUGUGAUUUGUGAACUUUACAUCUAGCUCUUGCAUAACGCCUUGUUUCAUCUAGUCG | |
| | CCUUCCAUAUAAUAUCUAAAGCUUUCAACCCUUGAGUUAACUCACUAGCAAUUUUGAGCUCAAAUAA | |
| | CCUAGAUAUAGAUA | |
| 371 | CUAAAACAGCCUGCGGGUUGUACCCGCCCGCAGGGCCCACUGGGCGCUAGUACUCUGGUACGCUAGU | enterovirusG2; |
| | ACACUAGCACGUUAGUGCUUUUGUACGCCAGUACCUUUGUACGCCUGUUUUAUUUUCCCUUCCCCAC | former- |
| | AUGUAACUUUAGAAGUUUAGAAACAAACAAUGACCAAUAGGAGUCCAGCAUCCAGUUGGAUUGCGGU | lyenterovirus10 |
| | CAAGUACUUCUGUUUCCCCGGACCUAGUAGUGAUAGGCUGUACCCACGGCCGAAGAUGAACCCGUCC | |
| | GUUAUCCGGCUACCUACUUCGGGAAGCCUAGUAACAUUCUGAAGUCUCUGAGGCGUUUCGCUCAGCA | |
| | CGACCCCGGUGUAGAUCGGGCUGAUGGGUCUCCGCAUACCCCACGGGUGACCGUGGCGGAGGCCGCG | |
| | UUGGCGGCCCGCCUAUGGCGAAAGCCAUAGGACGCCUCUUAGAUGACAGGGUGUGAAGAGCCUACUG | |
| | AGCUGGGUAGUAGUCCUCCGGCCCCUGAAUGCGGCUAAUCCUAACCACGGAGCGUCCACCGGCAAAC | |
| | CGGUCGGGAGGGCGUCGUAACGGGUAACUCUGUGGCGGAACCGACUACUUUGGGUGUCCGUGUUUCC | |
| | UUUUGAUCCUAUUUUGGCUGCUUAUGGUGACAACGAUAAGUUGUUAUCAUAAAGCUUUUGGGUUGGC | |
| | CACCUGGAAAAAGUUAUCAGUGUUUGAUAUUGUUCGGCUCUCACGCCUUCCAACAAAACAGGCCUUA | |
| | CUUCUUUUAAUUGCAUUUUAUCCAUCGGAGGUUAUUACAGAUUUCCAUCUGAAGCUUCUUCUCACAG | |
| | UAGCAGUAAA | |
| 372 | UUAAAACAGCCUGCGGGUUGUUCCCACCCGCAGGGCCCACUGGGCGCUAGUGCACUGGUAUGCUAGU | enterovirusG3 |
| | ACCUUGGUAUUACGGUACCAUUGUACGUUAGUACCUUUGCACGCCUGUUUUAAAUCCCCUCCCCCAU | |
| | GCAACAUUAGAAGUCUAUCUAUUAAGGACCAAUAGGAGUCCAACAGCCAGUUGGAUUGUGGUCAAGU | |
| | ACUUCUGUCUCCCCGGACUUAGUAGUGAUAGGCUGUACCCACGGCCGAAGAUGAACCUGUCCGUUAU | |
| | CCGGCCAGCUACUUCGAGAAGCCUAGUAGCACCAAAGAUCUGUCUUGGCGUUUCGCUCAGCGCGUCC | |
| | CCCCGCGUAGAUCGGGCUGAUGGGUCUCCGCAUCCCCCACGGGUGACCGUGGCGGAGGCCGCGUGGC | |
| | GGCCCGCCUAUGGCGAAAGCCAUAGGACGCCAUUUCAGUGACAGGGUGUGAAGAGCCUAUUGAGCUA | |
| | GUUGGUAGUCCUCCGGCCCCUGAAUGCGGCUAAUCCUAACCACGGAGCGUCCACCGGCAAACCAGCU | |
| | GGGAGGGCGUCGUAACGGGUAACUCUGUGGCGGAACCGACUACUUUGGGUGUCCGUGUUUCCUUUUG | |
| | AUCCUAUUGUGGCUGCUUAUGGUGACAACUUACAGUUGUUAUCAUAAAGCUUUUGGAUUGGCCAUCC | |
| | GGAAAGAGUUAACAGUUUGUUCGCUCAUUGCUCUCUCAAAGUUUCCAUUAAGGAAAGCAAUUUACCU | |
| | UCUUAUUUCAUUUUACAGGCUUGACAAGAUUUCAAGCGCUCAUAUAGAUCUGCUUAUUCAGCUACAU | |
| | CAAAC | |
| 373 | UUAAAACAGCCUGUGGGUUGUUACCACCCACAGGCGCCACGGGGCGCUUCGCACCUGGGAUGCUUAA | enterovirusG4 |
| | CGUCGGAUUUGCGGAUCCGAUGUUUAUUGCCCCUGUGCGCGCCUGUUAUAUUCCCUUCCCCCUUAAG | |
| | UAACUUUAGAAGUUUAUAAUAACAAAGACCAAUAGGAGUCCAACAUCCAGUUGGAUCGCGGUCAGGC | |
| | ACUUCUGUCUCCCCGGACCUAGUAGUGAUAGGCUGUACCCACGGCCGAAGAUGAACCCGUCCGUUAU | |
| | CCGGCCAGCUACUUCGAGAAGCCUAGUAACAUCAAAGAUCUGUCCUGGCGUUUCGCUCAGCGCGUUU | |
| | CCCCCCGCGUAGAUCGGGCUGAUGGGUCUCCGCAUACCCCACGGGCGACCGUGGCGGAGGCCGCGUG | |
| | GCGGCCCGCCUAUGGCGAAAGCCAUAGGACGCCAUUUCAGUGACAGGGUGUGAAGAGCCUAUUGAGC | |
| | UAGUUGGUAGUCCUCCGGCCCCUGAAUGCGGCUAAUCCUAACCACGGAGCGUCCACCAGCAAACCAG | |
| | CUGGAAGGGCGUCGUAACGGGUAACUCUGUGGCGGAACCGACUACUUUGGGUGUCCGUGUUUCCUUU | |
| | UGAUCCUAUUGUGGCUGCUUAUGGUGACAACUUACAGUUGUUAUCAUAAAGCUUUUGGAUUGGCCAU | |
| | CCGGAAAGAGUUAACAGUUUGUUCGCUUAUUGCUCUCUCAAAACUUCCAUUGAGGAAAGCAAUUUAU | |
| | CUUCUCAUUUCAUUUUACAAGCUUGAUAAAAUUUCAAGUGCUCAUUUAGAGUUACUCAUCCAACUCC | |
| | AUCAAAU | |
| 374 | UAAACAGUCAGGGGGUUGUACCCACCUCCGGGGCCCACGUGGCGUCAGUGCUCUGGUGCGUUAGUAC | enterovirusG5 |
| | CUUUGCACGCCUGUUUUCCCUUCCCUUAAACAAAUUAAGAUCGCCAGCGCCGAGGGGAGUAGUCCGA | |
| | CUCCGCUCCACUACGGUGCUCCAGUGGAUUGCUAUUACGGUAGCUCUUCACGGGGUGGCUGGUGUCC | |
| | CCAACCCCGCAACUUAGAAGCCAGCACAGAUCGACCAAUAGCGGCUCGGCAGCCAGUCGGGCAACGG | |
| | UCAAGCACUUCUGUUUCCCCGGUCCGUAAGGAUCGUUACCCGCCCAGGAUGCUACGAGAAACUUAGU | |
| | AAACACCCAAGCUUGUGAGAGGUUGCGCUCAGCCACAACCCCAGUGGUAGCUCUGAGUGAUGGGGCU | |
| | CGUGACUACCCCUCUGGUAACAGAGACACUUGCCCGCGUGUGCACUCGGGUUCAUCAGAAAUGGUGU | |
| | UCACCUCGACCAAGCGUAAGUGUUGAAGAGUCUAUUGUGCCAGCCUGGUUUCCUCCGGGGCCGUGAA | |
| | UGCUGCUAAUCCUAACCUCCGAGCGUGUGCGCACAAUCCAGUGUUGCUACGUCGUAACGCGUAAGUU | |
| | GGAGGCGGAACAGACUACUUUCGGUACCCCGUGUUUCCUCAUUCAUUAUUUCAAUUUAUGGUGACAA | |
| | UUACAGAGAUUUGUGAAUUGGCGAAACUACCACUGAAUUUGGCCUUGUAUUGUCUUAUUGCCUUCCA | |
| | CAAAACUUCUGAUAUUCCACCCAGUUAUAUCGAUCUCCUGGUUUAUCUAAAUUUACAAUAUAAAUUG | |
| | AACCAGAUACA | |
| 375 | UUAAAACAGCCUGUGGGUUGUUCCCACCCACAGGGCCCACUGGGCGCUAGUACACUGGUAUGCCUUA | enterovirusG6 |
| | CCUCGGUAUCACGGUACCGUUGUAUACAGUACCUUUGUACGCCUGUUUUAAAUCCCCUCCCCCCAAU | |
| | GUAACUUUAGAAGUUUAAGCAAACAAAGACCAAUAGGAGUCCAACAACCAGUUGGAUUGCGGUCAAG | |
| | CACUUCUGUCUCCCCGGACCUAGUAGUGAUAGGCUGUACCCACGGCCGAAGAUGAAACCGUCCGUUA | |
| | CCCGGCCAGCUACUUCGAGAAGCCUAGUAACAUCAAAGAUCUGUCUUGGCGUUUCGCUCAGCGCGUU | |
| | CCCCCCGCGUAGAUCGGGCUGAUGGGUCUCCGCAUACCCCACGGGCGACCGUGGCGGAGGCCGCGUG | |
| | GCGGCCCGCCUAUGGCGAAAGCCAUAGGACGCCAUUUCAGUGACAGGGUGUGAAGAGCCUAUUGAGC | |
| | UAGUUGGUAGUCCUCCGGCCCCUGAAUGCGGCUAAUCCUAACCACGGAGCGUCCACCAGCAAACCAG | |
| | CUGGCAGGGCGUCGUAACGGGUAACUCUGUGGCGGAACCGACUACUUUGGGUGUCCGUGUUUCCUUU | |
| | UGAUCCUAUAUUGGCUGCUUAUGGUGACAACGAUAAGUUGUUAUCAUAAAGCUUUUGGGUUGGCCAC | |
| | CUGGAAAAAGUUAUCAGUGUUUGAUAUUGUUCGGCUUUCACGCCUACCAAUAAAACAAUCCUUAUAU | |
| | UAUUUGAUUUCCUUUUAUCCACAAGAGGAAAUUUCUGAGUUUCAUCUCCAGUUGCUCUUAACCUUAU | |
| | CAGCAAG | |
| 376 | UAUUUUCCAGGUGGAAUGGGGAUGCAGGCCUGAGUUGUUUUUGCCCCCAAGGAAUAAAUCAAUAUUC | enterovirusG7 |
| | CCAGAGCCGAGGGGAGUAGUCCGACUCCGCUCCACUACGGUGCUCCAGUAGAUUGCUAUUGCGGUAG | |
| | CUCUCUACGGGGUGGCUGGUAUUCCUCACCCUGAAACUUAGAAGUUUGUACACACCGACCAAUAGCG | |
| | GUCUGGCAGCCAGCCAGGCAACGGUCAAGCACUUCUGUUUCCCCGGUCCGCAAGGAUCGUUACCCGC | |
| | CCGAAUUGCUACGAAAAGCCUAGUAAGUACCCAAGUUUGUGCGAGGUUGCGUUCAGCCACAAUCCCA | |
| | GUGGUAGCUCUGAGUGAUGGGGCUCGUGAUUACCCCUCUGGUGACAGAGACACUUGCCCGCGCGUGC | |
| | ACUUGGGUUCACCAGAAAUGGUGUUCACCUCAAUCAAGCGCAAGUACUGAAGAGCCAAUUGUGCCAA | |
| | UUUGGUCUCCUCCGGGGCCGUGAAUGCUGCUAAUCCUAACCUCCGAGCGUGUGCGCACAAUCCAGUG | |
| | UUGCUACGUCGUAACGCGUAAGUUGGAGGCGGAACAGACUACUUUCGGUACCCCGUGUUUCCUUAUU | |
| | UUAUUCAUUUAAAUUUAUGGUGACAAUUGUUGAGAUUUGUGAGCUAGCAAAAUUACCAUUGAAUUUG | |
| | GCAUUGUACUGUCUCAUUGCUUUUCACAAAACCUCAGAUAUCCCACCCAGUUACAUUGAUCUUUUAG | |
| | UUCAUUUGAAUUUACAGUUCAAACUGAGACAAGUAAA | |
| 377 | UACCUUUGUACGCCUGUUUUAUACUCCCUUCCCCUACAUGUAACCUUAGAAGUAUAAAUAAACAAAG | enterovirusG8 |
| | ACCAAUAGGAGUCCAACAUCCAGUUGGAUCGCGGUCAAGUACUUCUGUUUCCCCGGACCUAGUAGUG | |
| | AUAGGCUGUACCCACGGCCGAAGAUGAACCCGUCCGUUAUCCGGCCAGCUACUUCGAGAAGCCUAGU | |
| | AACAUCAAAGAUCUGUCUUGGCGUUUCGCUCAGCGCGUUCCCCCCGCGUAGAUCGGGCUGAUGGGUC | |
| | UCCGCAUACCCCACGGGCGACCGUGGCGGAGGCCGCGUGGCGGCCCGCCUAUGGCGAAAGCCAUAGG | |
| | ACGCCAUUUCAGUGACAGGGUGUGAAGAGCCUAUUGAGCUAGUUGGUAGUCCUCCGGCCCCUGAAUG | |
| | CGGCUAAUCCUAACCACGGAGCGUUCACCAGCAAACCAGCUGGGAGGGCGUCGUAAUGGGCAACUCU | |
| | GUGGCGGAACCGACUACUUUGGGUGUCCGUGUUUCCUUUUGAUCCUAUAUUGGCUGCUUAUGGUGAC | |
| | AACGAUAAGUUGUUAUCAUAAAGCUUUUGGGUUGGCCACCUGGAAAAAGUUAUCAGUGUUUGAUAUU | |
| | GUUCGGCUUUCACGCCUACCAAUAAAACAAGCUUUAUAUUAUUUAAUCUCCUUUUACCCAAAGGGAG | |
| | AGAUUUCUGAAUUUCAUAUCCAGUUGCUCCUAACACUAUCAGCAAA | |
| 378 | GCCUGUUUUAUAUUCCCCUCCCCCAAACGUAACAUUAGAAGUAUAAACAAACAAAGACCAAUAGGAG | enterovirusG9 |
| | UCCAACAUCCAGUUGGAUUGCGGUCAAGCACUUCUGUCUCCCCGGACCUAGUAGUGAUAGGCUGUAC | |
| | CCACGGCCGAAGAUGAACCCGUCCGUUAUCCGGCCAGCUACUUCGAGAAGCCUAGUAACAUCAAAGA | |
| | UCUGUCUUGGCGUUUCGCUCAGCGCGUUCCCCCCGCGUAGAUCGGGCUGAUGGGUCUCCGCAUACCC | |
| | CACGGGCGACCGUGGCGGAGGCCGCGUGGCGGCCUGCCUAUGGCGAAAGCCAUAGGACGCCAUUUCA | |
| | GUGACAGGGUGUGAAGAGCCUAUUGAGCUAGUUGGUAGUCCUCCGGCCCCUGAAUGCGGCUAAUCCU | |
| | AACCACGGAGCAUCCACCAGCAAACCAGCUGGAAGGGUGUCGUAACGGGUAACUCUGUGGCGGAACC | |
| | GACUACUUUGGGUGUCCGUGUUUCCUUUUGAUCCUAUAUUGGCUGCUUAUGGUGACAACGAUAAGUU | |
| | GUUAUCAUAAAGCUUUUGGGUUGGCCACCUGGAAAAAGUUAUCAGUGUUUGAUAUUGUUCGGCUUUC | |
| | ACGCCUACCAAUAAAACAAGCUUUAUACUUGCUAAUUUCAUUUUAUCCAUCUGAGGAUAUUACUGAA | |
| | UUUUACCUACAACUUUUGCUCACCCUAACAGUAAA | |
| 379 | GGGUUGUUCCCACCCGCGGGGCCCACUGGGCGCUAGUACACUGGUACGCCAGUACCUUGGUAUUACG | enterovirusG10 |
| | GUACCAUUGUACGUCAGUACCUUUGUACGCCUGUUUUAUAUCCCUUCCCCCACAUGUAACAUUAGAA | |
| | GUCAAGUAAACAAAGACCAAUAGGAGCCCAACAACCAGUUGGGUUGCGGUCAAGCACUUCUGUCUCC | |
| | CCGGACCUAGUAGUGAUAGGCUGUACCCACGGCCGAAGAUGAACCCGUCCGUUAUCCGGCCAGCUAC | |
| | UUCGAGAAGCCUAGUAACAUCAAAGAUCUGUCUUGGCGUUUCGCUCAGCGCGUUCCCCCCGCGUAGA | |
| | UCGGGCUGAUGGGUCUCCGCAUACCCCACGGGCGACCGUGGCGGAGGCCGCGUGGCGGCCCGCCUAU | |
| | GGCGAAAGCCAUAGGACGCCAUUUCAGUGACAGGGUGUGAAGAGCCUAUUGAGCUAGUUGGUAGUCC | |
| | UCCGGCCCCUGAAUGCGGCUAAUCCUAACCACGGAGCAUCCACCAGCAAACCAGCUGGGAGGGUGUC | |
| | GUAACGGGUAACUCUGUGGCGGAACCGACUACUUUGGGUGUCCGUGUUUCCUUUUGAUCCUAUUUUG | |
| | GCUGCUUAUGGUGACAACGAUAAGUUGUUAUCAUAAAGCUUUUGGGUUGGCCACCUGGAAAAAAUUG | |
| | UCAGUGUUUGAUAUCGCUCGGCUUUCACGCCUACCAGUCAAACAAGCUCUUUAUCUCCUUGUUGGAU | |
| | UCUACUCAAUAAAGGACAUAACUCAGUUUCACAUAAAUCUGUUGCUUUCAUUGUCAGCAAA | |
| 380 | UUAAAACAGCCUGUGGGUUGUUCCCACCCGCAGGGCCCACUGGGCGCUAGUACACUGGUAUGCCAGU | enterovirusG17 |
| | ACAUUAGUACGCUAGUACUCUUGUAUGCCAGUACCUUUGUACGCCUGUUUUAUACUCCCCUCCCCAU | |
| | AUGUAACAUUAGAAGUUUAAGUAAUAAAGACCAAUAGGAGUCCAACAUCCAGUUGGAUUACGGUCAA | |
| | GCACUUCUGUUUCCCCGGACCUAGUAGUGAUAGGCUGUACCCACGGCCGAAGAUGAACCCGUCCGUU | |
| | AUCCGGCCAGCUACUUCGAGAAGCCUAGUAACAUCAAAGAUCUGUCUUGGCGUUUCGCUCAGCGCGU | |
| | UCCCCCCGCGUAGAUCGGGCUGAUGGGUCUCCGCAUACCCCACGGGUGACCGUGGCGGAGGCCGCGU | |
| | GGCGGCCCGCCUAUGGCGAAAGCCAUAGGACGCCAUUUCAGUGACAGGGUGUGAAGAGCCUAUUGAG | |
| | CUAGUUGGUAGUCCUCCGGCCCCUGAAUGCGGCUAAUCCUAACCACGGAGCGUCCACCAACAAUCCA | |
| | GUUGGCAGGGCGUCGUAACGGGUAACUCUGUGGCGGAACCGACUACUUUGGGUGUCCGUGUUUCCUU | |
| | UUGAUCCUAUAUUGGCUGCUUAUGGUGACAACGAUAAGUUGUUAUCAUAAAGCUUUUGGGUUGGCCA | |
| | CCUGGAAAAAGUUGUCAGUGUUUGAUAUUGUUCGGCUUUCACGCCUACCAAUAAAGCAAGCUUUAUA | |
| | UUUGUUAGUUUCAUUUUAUCCAUUAAAGGAUAUUACCGAAUUUCACUUACAGCUUUUGCUCACAUUA | |
| | UCAGCAAA | |
| 381 | UUUAAAACCAACCCGGGGGUUGUACCCACCCCUGGGGCCCACGUGGCGGUAGUGCUUUGGUUUGCUC | enterovirusG20 |
| | AGAACCUUUGCACGCCUGUUUUCCCUUCCCUUAAAUAAAUUAAGCUUGCCGGGACUGAGGGGAGUAC | |
| | UCCGACUUCGCUCCCCUACGGUGCCCCAGUAAACUGGUAUCGCGGUACCUUUUUACGGAGUCACCGG | |
| | CACCCCCCCCCCGCAACUUAGAAGUUUGAACACAAACCGACCAAUAGAGGCCCGGCAUCCAGUCGAG | |
| | CAACGGUCAAGCACUUCUGUUUCCCCGGUCCGCAAGGAUCGUUACCCGCCCCGACUACUACGGGAAG | |
| | CCUAGUAUCUAGCCAAACUUGGAAAAGGUUGCGCUCAGCCACAACCCCAGUGGUAGCUCUGAGUGAU | |
| | GGGGCUCGCGACAACCCCCUUGGUAACAAGGACGCUUGCCCGCGCGUGCACUCGGGUUCACCUUCCG | |
| | GUGUUCACCCUGACCAAGUGUAACUAGAGAAGCGCCUACUGUGCUAGUUGGGUUUUCCUCCGGGGCC | |
| | GUGAAUGCUGCUAAUCCCAACCUCCGAGCGUGUGCGCACAAUCCAGUGUUGCUACGUCGUAACGCGC | |
| | AAGUUGGAGGCGGAACAgACUACUUUCGGUACCCCGUGUUUCCUUUAUUUGUUUAUUAAAUUUAUGG | |
| | UGACAAUUGUUGAGAUUUGUGAUUUAGCGAAAUUACCCUUGAAUUUGGCAUUAUAUUACUUGGUUGC | |
| | AUUUCACAAAACCUCAaAGAUUCCCGCAUCCUAUAUAGAUCUUCUCGUCUAUUUGAACUUACAGCAC | |
| | AAAUUAAAUCAAAUAAA | |
| 382 | UUAAAACAGCCUGGGGGUUGUUCCCACCCCCAGGGCCCACGUGGCGCAAGUACUCUGCUUCGUUAGA | enterovirusI2; |
| | AGCCUUGUACGCCUGUUUUCCCCUCCUUAAAUAAAUUAAGAUUACCACUUCCGAGGGGAGUAGUCCG | dromedary- |
| | ACUCCGCUCCGAUACGUCGCUCCAGUGCGCUAGUACGCUUAGUACUUUCUCACGGGGAUAGUGGUAC | camelenterovirus2 |
| | CCCUCCCUAGUAAUUUAGAAGACUUGAAAAACCGACCAAUAGGCACCUCGCAUCCAGCGGGGUAAAG | |
| | GUCAAGCACUUCUGUUUCCCCGGGUCGAGUAGCGAUAGACUGUGCCCACGGUCGAAGGUGAAACAAC | |
| | CCGUUAUCCGACUUUGUACUUCGGGAAGCCUAGUACCACCAAAGAUUAUGCUUGGGGUUUCGCUCAG | |
| | CACGACCCUGGUGUAGAUCAGGCUGAUGGAUCACCGCGUUCCUCACAGGCGACUGUGGCGGUGGUCG | |
| | CGUGGCAGCCUGCCGAUGGGGCAACCCAUCGGACGCCAAGCAUAUGACAGGGUGUGAAGAGCCUACU | |
| | GAGCUACAAAGUAUUCCUCCGGCCCCUGAAUGCGGCUAAUCCCAACCACGGAGCAUUUGCUACCAAA | |
| | CCAGGUAGUGGAAUGUCGUAACGGGUAACUCUGUGGCGGAACCGACUACUUUGGGUGUCCGUGUUUC | |
| | CUUUUAAUUUAUCAUUCUGUGUAUGGUGACAACUAUAGUGCUAUCUCGAUUUGCAUUACUAUUGUUG | |
| | AGAUUGACACUUUAUUACAUUGUUGCAUUUUACCCUUUGAGUGAGUUUUCACCUGAACAGAUUAAUU | |
| | UACUUAUCCUGUUUAUAUAUCACAAGCAGAAAUACUUGCAAAG | |
| 383 | AAAAUAGCCUCAGGGUUGUUCCCACCCUGAGGGCCCACGUGGCGUAGUACUCUGGUAUCACGGUACC | enterovirusJ103 |
| | UUUGUACGCCUAUUUUACAUCCCUUCCCCGUAACUUAGAAGCUUUCAACUAUAAGUUCACUAGCAGG | |
| | GCUACCAACCAGGUAGUUCAUCGAACAAGCACUUCUGUUACCCCGGUCCUAGUACCUGAGAAGCUGU | |
| | ACCCACGGCGGAAGGGGAAAAAGAUCGUUACCCGGCUAUCUACUUCGAGAAGCCUAGUAACGUCAUU | |
| | GAAACUCUUGAGCGUUGCGUUCAGCACAGUACCCCCUGUGUAGCUUUGGCUGAUGAGUCUGGGCAUU | |
| | CCCCACUGGCGACAGUGGCCCAGGCUGCGUUGGCGGCCAACCGAUCCGGGCAACCGGGUCGGACGCU | |
| | CAUUUGUGGACAUGGUGUGAAGAGCCUAUUGAGCUAGGUAGUAGUCCUCCGGCCCCUGAAUGCGGCU | |
| | AAUCCCAACUCCGGAGCAAGUGCGCACAAUCCAGUGUGUGGCUUGUCGUAAUGCGUAAGUCUGGAGC | |
| | GGAACCGACUACUUUGGGUGUCCGUGUUUCCUGUUUUUCCUUAUUUGGCUGCUUAUGGUGACAAUCU | |
| | AGUGUUGUUACCAUAUAGCGAUUGGAUUGGCCAUCCGGUGAAUAGCUUUGUGACAAUUCCCUACGUU | |
| | AUUUUUCAUAACUCAACUUCACGAGAGUCAUUGAGUUUGACUAAUU | |
| 384 | UUAAAAGAGCCUGGGGGUGACUCCAGCCCCCAGGACCCACGUGGUGCCAGCACUACUUAGUUACGAU | enterovirusk1 |
| | UAUUUAGUGCGCCUCUUUUAUAUCCCCUCCCCCACCCUUCCCUUUUCCUUGAAUUCUUUCGUAACAA | |
| | GUGCCUCAGCAUGACGUUUGCACGGUGUACUGUAACCAUCAGGCAACCAGCCUGUAAAGCAGGAAGC | |
| | ACUUCUGUUUCCCCGGUGUGGAUAUUAGGCUGUUCCCACGGCCGAAAAUGAAGCAUCGUUAUCCCCA | |
| | CUGGUACUGCGCGAAAGCUAGUAACACCAAUGAUUUAGAUGGAUCUUUCGCUCAGCAAUUGCCCCGA | |
| | UUGUAGAUCGGGCGGAUGGGGCUCCUCGUUCCUCACUGGCGACAGUGGAGGAGCCCGCGUGGCCGCC | |
| | CGCCUACCCCGCAUGGGGUAGGAGGUCCAGAUAAUGACAGGGUGUGAAGACUCAAGUGCGCUACCAG | |
| | UGCAUCCUCCAGGCCUCUGAAUGCGGCUAAUCCUAACCCCGGAGCACCCGCUCACAGUCCAGUAAGU | |
| | AGGGUGUCGUAACGCGUAAGUCUGGGAUGGAACCGACUACUUUGAGUGACCUGUGUUUCUUUUAUUU | |
| | UUAUUGAGGUUGUUUAUGGUGACAAAGAUUGGCUCUAUCGUGUAUAGAGUUUACAAGGUGAAAGAGG | |
| | AUUUUUACAGACUUUUGUUAUCAAUCUUGUCCUAUCCUACCCGCAGGGGAAUUGAUGAUAGAAGUGC | |
| | UUUUAGAGGUGAUUCAAUUGAUAGCUCAAACAACUGAGAAUCUUAUUUAUUUUUAUCAAGGUCUGAA | |
| | UUUGAAUAAAGAUCCAGUGAAUUUUACCCCCACCUAUUAGCGACCAAAUUCACAAGCA | |
| 385 | UUAAAACUGGGUGUGGGUUGUUCCCACUCACACCACCCAAUGGGUGUUGUACUCUGUUAUUCCGGUA | rhinovirusA1B |
| | ACUUUGUACGCCAUUUUCCCUCCCUCCCCAUCCUUUUACGUAACUUACAACUUUUAAACACAAGACC | |
| | AAUAGUAGGCAACUCUCCAGGUUGUCUAAGGUCAAGCACUUCUGUUUCCCCGGUUGACGUUGAUAUG | |
| | CUCUAACAGGGCAAAAACAACUGAUAUCGUUACCCGCAAAGUGCCUACACAGAGCUUAGUAGGAUUC | |
| | UGAAAGAUCUUUGGUUGGUCGCUCAGCUGCAUACCCAGCAGUAGACCUUGCAGAUGAGGCUGGACAU | |
| | UCCCCACUGGUAACAGUGGUCCAGCCUGCGUGGCUGCCUGCACACUCUUAUGAGGUGUGAAGCCAAA | |
| | GAUUGGACAGGGUGUGAAGAGCCGCGUGUGCUCACUUUGAGUCCUCCGGCCCCUGAAUGCGGCUAAC | |
| | CUUAAACCUGCAGCCAUGGCUCAUAAACCAAUGAGCUUAUGGUCGUAAUGAGCAAUUGCGGGAUGGG | |
| | ACCGACUACUUUGGGUGUCCGUGUUUCACUUUUUCCUUUAUCAAUUGCUUAUGGUGACAAUAUAUAC | |
| | AUAGAUAUAUAUUGGCAUC | |
| 386 | UUAAAACUGGAUCCAGGUUGUUCCCACCUGGAUUUCCCACAGGGAGUGGUACUCUGUUAUUACGGUA | rhinovirusA2 |
| | ACUUUGUACGCCAGUUUUAUCUCCCUUCCCCCAUGUAACUUAGAAGUUUUUCACAAAGACCAAUAGC | |
| | CGGUAAUCAGCCAGAUUACUGAAGGUCAAGCACUUCUGUUUCCCCGGUCAAUGUUGAUAUGCUCCAA | |
| | CAGGGCAAAAACAACUGCGAUCGUUAACCGCAAAGCGCCUACGCAAAGCUUAGUAGCAUCUUUGAAA | |
| | UCGUUUGGCUGGUCGAUCCGCCAUUUCCCCUGGUAGACCUGGCAGAUGAGGCUAGAAAUACCCCACU | |
| | GGCGACAGUGUUCUAGCCUGCGUGGCUGCCUGCACACCCUAUGGGUGUGAAGCCAAACAAUGGACAA | |
| | GGUGUGAAGAGCCCCGUGUGCUCGCUUUGAGUCCUCCGGCCCCUGAAUGUGGCUAACCUUAACCCUG | |
| | CAGCUAGAGCACGUAACCCAAUGUGUAUCUAGUCGUAAUGAGCAAUUGCGGGAUGGGACCAACUACU | |
| | UUGGGUGUCCGUGUUUCACUUUUUCCUUUAUAUUUGCUUAUGGUGACAAUAUAUACAAUAUAUAUAU | |
| | UGGCACC | |
| 387 | UUAAAACUGGGAGUGGGUGUUCCCACUCACUCCACCCACGUGGUGCUGUACACUGUUAUUCCGGUAA | rhinovirusA7 |
| | CUUUGUACGCCAGUUUUUCCCUCCCUUCCCUCAUUAUGUAACUUAGAAGCAUGAACAUAUGGACCAA | |
| | UAGGCAAUAAUCAUCCAGAUUAUUGAAGGUCAAGCACUUCUGUUUCCCCGGUCAAUAAAGAUAUGCU | |
| | UUACCCAAGGCAAAAACUUUUGAGAUCGUUACCCGCACAUCAACUACGCAAAGCCUAGUAUCACCUU | |
| | UGAUGUACUAAGACUGGUCGUUUCCUGCAACCCCAGCAGUAGACCUGGCAGAUGAGGCUGGACGUAC | |
| | CCCACUGGCGACAGUGGUCCAGCCUGCGUGGCUGCCUGCUCGCCCAUAUGGGCGAGAAGCCUUAUUA | |
| | UUGACAAGGUGUGAAGAGCCGCGUGUGCUUGGUGUGAGUCCUCCGGCCCCUGAAUGUGGCUAACCUU | |
| | AACCCUGCAGCCAUUGCUCAUAAUCCAAUGAGUUAGUGGUCGUAAUGCGCAAGUGCGGGAUGGGACC | |
| | AACUACUUUGGGUGUCCGUGUUUCUUAUUUUUUCUUUCAUGUGUGUUAUAUGGUGACAAUUUAUAGU | |
| | AUAUAAGUUGUCAUC | |
| 388 | UUAAAACUGGGUUCAGGUUGUUCCCAUCUGAACUUCCCACGUGGAGUAGUACACUAUUAUUACGGUA | rhinovirusA8 |
| | AUUUUGUACGCCAGUUUUACCUCCCUCCCCAAAUGUAACUUAGAAGAUGUACACAACGACCAACAGG | |
| | CGGUGGUUAUCCAAACCAUCAAAGGUCAAGCACUUCUGUUUCCCCGGUCAAUGUUGAUAUGCUCCAA | |
| | CAGGGCAAAAACAACUGCGAUCGUUAUCCGCAAGAUGCCUACGCAAAGCUUAGUAACACUUUGGAAG | |
| | AUUUAUGGCUGGUCGUUCCACUAUACCCCAUAGUAGACCUGGCAGAUGAGGCUAGAAAUACCCCACU | |
| | GGUGACAGUGUUCUAGCCUGCGUGGCUGCCUGCGCACCCUAUGGGUGCGAAGCCAUACAUUGGACAA | |
| | GGUGUGAAGAGCCCCGUGUGCUCAUCUUGAGUCCUCCGGCCCCUGAAUGUGGCUAACCUUAACCCCG | |
| | CAGCCAGUGCACACAAUCCAGUGUGUUUUUGGUCGUAAUGAGUAAUUGUGGGAUGGGACCAACUACU | |
| | UUGGGUGUCCGUGUUUCACUUUUUCCUUUAUUAUGCUUAUGGUGACAAUAUAUACAUAUAUAUUGAC | |
| | ACC | |
| 389 | UUAAAACUGGAUCUGGGUUGUUCCCACUCAGAUCUCCCACGUGGAGUAGUACACUAUUAUUACGGUA | rhinovirusA9 |
| | AUCUUGUACGCCAGUUUUAUAAUCCCCUUACCCAAGUAACUUAGAAGAUAAACACAAAGACCAAUAG | |
| | GAGAUGAUCAUCCAGAUCAUCAUAGGUCAAGCACUUCUGUUUCCCCGGUCAAGGUUGAUAUGCUCUA | |
| | ACAGGGCAAAAACAGCUGAGAUCGUUAUCCGCAAAGCGCCUACGCAAAGCCUAGUAACUAUCUGGAA | |
| | GUUGCUUGGUUGGUCGCUCCGCCAUAUCCCAUGGUAGACCUGGCAGAUGAGGCUAGAAAUUCCCCAC | |
| | UGGUGACAGUGUUCUAGCCUGCGUGGCUGCCUGCACACCCUCUGGGUGUGAAACCAAGUAAUGGACA | |
| | GGGUGUGAAGAGCCCCGUGUGCUCGCUUUGAGUCCUCCGGCCCCUGAAUGUGGCUAACCUUAACCCU | |
| | GCAGCUAGGGCACACAAUCCAGUGUGUAUCUAGUCGUAAUGAGCAAUUGCGGGAUGGGACCAACUAC | |
| | UUUGGGUGUCCGUGUUUCUUGUUUUUCUUUUAUGUUUGCUUAUGGUGACAAUAUAUAGUAAUAUAUa | |
| | UUGGCAUC | |
| 390 | UUAAAACUGGAUCCAGAUUGUUCCCAUCUGGAUUUCCCACGUGGAGUUGUACACUAUUAUUCCGGUA | rhinovirusA10 |
| | AUUUUGUACGCCAGUUUUAUGUACCCCCCCCUGUAACUUAGAAGUUUUGAACAAGGACCAAUAGGCA | |
| | GUAACCACCCAGGUUGCUAAAGGUCAAGCACUUCUGUUUCCCCGGUCAAAGUUGAUAUGCUCCAACA | |
| | GGGCAAAAACAACUGAGAUCGUUACCCGCAAGGUGCCUACGCAAAGCCUAGUAACCCCUUGAACGAU | |
| | GUGUGGUUGGUCGUUCGGCUGUACCCCACAGUAGACCUGGCAGAUGAGGCUAGAUGUUCCCCACUGG | |
| | CGACAGUGUUCUAGCCUGCGUGGCUGCCUGCGCACUCUCUGAGUGCGAAGCCAUACAUUUGACAAGG | |
| | UGUGAAGAGCCCCGUGUGCUCACCUUGAGUCCUCCGGCCCCUGAAUGUGGCUAACCUUAACCCUGCA | |
| | GCUAGUACAUGCAAUCCAGCAUGCAGCUAGUCGUAACGAGCAAUUGCGGGACGGGACCAACUACUUU | |
| | GGGUGUCCGUGUUUCACUUUUUCCUUUUAUAUUUGCUUAUGGUGACAAUAUAUAGAUUUAUAUAUUG | |
| | UCACC | |
| 391 | UUCAAACUGGAUCCAGGUUGUUCCCACCUGGAUCUCCCACGUGGAGUUGUACUCUAUUAUUCCGGUA | rhinovirusA11 |
| | AUUUUGUAUGCCAGUUUUAUCUCCCCUUCCCUUUUGUAACUUAGAAGAUGAACUCACCGACCAAUAG | |
| | GUGGCAUUUAUCCAGAAUGUCAAAGGUCAAGCACCUUCUGUUUCCCCGGUCAAAGUUGAUAUGCUCC | |
| | AACAGGGCAAAAACAUCUGCGAUCGUUAUCCGCAAAGCAACUACGCAAAGCCUAGUAUCACUUUUGA | |
| | GAGUAUGUGGUUGGUCGCUCCACUAUAAACCCAAUAGUAGACCUGGCAGAUGAGGCUAGAAGUCCCC | |
| | CACUGGUAACAGUGUUCUAGCCUGCGUGGCUGCCUGCGUGCCUCUGGUAGGCACGAAGCCAUAUAUU | |
| | UGACAAGGUGUGAAGAGCCCCGUGUGCUUGCUUUGAGUCCUCCGGCCCCUGAAUGUGGCUAACCUUA | |
| | ACCCUGCAGCUAGUGUGCAUAAUCCAAUGCAUGGCUAGUCGUAAUGAGCAAUUGCGGGAUGGGACCA | |
| | ACUACUUUGGGUGUCCGUGUUUCUCAUUUUUCCUUUUAUUUGCUUAUGGUGACAAUAUAUAUAAUAC | |
| | AUAUAUAUUGGCAUC | |
| 392 | UUCAAACUGGGUUGUGGUUGAUCCCACCACAACUGCUCACUGAGCGUAGUGCUCUCAUAUUCCGGUA | rhinovirusA12 |
| | UGCUUGCACGCCAGUUUUACUCCCCUCCCCUCAUUGUAACUUAGAAGCAAAGCACACCUGAUCAAUA | |
| | GGAGAUAACUUGCCAGGUUAUCUAAGAUCAAGCACUUCUGUUUCCCCGGCAAGUGUGAAUAUGCUUU | |
| | ACCCAAGGCAAAAGCUGAUCUUGUCGUUAUCCGCAAGGUGCCUACGAGAAGCCUAGUAAUGCUCAAG | |
| | AUGUGGUUUGACUGGUCGCUCCGCUGUUCCCCACAGUAGACCUGGCAGAUGAGGCUGGACAUUCCCC | |
| | ACCAGCGAUGGUGGUCCAGCCUGCGUGGCUGCCUGCACACCCUAUGGGUGUGAAGCCAAAUUUACUG | |
| | ACAGGGUGUGAAGAGCCUAUUGUGCUCACCUUGAUUCCUCCGGCCCCUGAAUGUGGCUAAUCCUAAC | |
| | CCCGCUGCUAUUGCACACGAACCAGUGUGUAGAUAGUCGUAAUGAGCAAUAGUGGGAUGGAACCAAC | |
| | UACUUUGGGUGUCCGUGUUUCUUGUUUUUCUUUAUUUGUCUUAUGGUCACAAUAUAUAUAUAACGUA | |
| | UAUUGUGAUC | |
| 393 | UUAAAACUGGGUUCGGGUUGUUCCCACCCAAACCACCCACGUGGUGUAGUACACUGUUAUCCCGGUA | rhinovirusA13 |
| | ACUUUGUACGCCAGUUUUAUCUCCCCACCCCCUUUUGUAACUUAGAAGUUAUGCACAAUGACCAAUA | |
| | GUGGGCAAUCAUCCAGAUUGUCAUCGGUCGAGCACUCCUGUUUCCCCGGUUCAAGUUGAUAUGCUCU | |
| | AACAGGGCAAAAACAAUUUGAAUCGUUACCCGCAAGGUGCCUACACAGAACUUAGUACCAUUCUGGA | |
| | AAAUUCUUGGCUGGUCGCUCAGUUACUACCCCAGUAAUAGACCUGGCAGAUGGGGCUAGAAGUUCCC | |
| | CACUGGCGACAGUGUUCUAGCCUGCGUGGCUGCCUGCACACCCUUCUUGGGUGUGAAGCCUAGAAUU | |
| | GGACAGGGUGUGAAGAGUCGCGUGUGCUCAUCUUGAGUCCUCCGGCCCCUGAAUGCGGCUAACCCUA | |
| | ACCCUGCAGCCAUUGCCCACAACCCAGUGGGUCUAUGGUCGUAAUGAGCAAUUGCGGGAUGGGACCG | |
| | ACUACUUUGGGUGUCCGUGUUUCACUUUUUACUUAUUAUUUGCUUAUGGUGACAAUAUAUAGAUACA | |
| | UAUAUUGACACC | |
| 394 | UUAAAACUGGGUUCAGGUUGAUCCCACCUGAACCUCCCAAGCGGAGUAGUACUCUGUUAUUUCGGUA | rhinovirusA15 |
| | ACUUUGUACGCCAGUUUUAUAUCCCCCUCCCCAUGUAACUUAGAAGAUGAGCUCACAGACCAAUAGC | |
| | CGGCAAUCAUCCAGAUUGUCAAUGGUCAAGCACUUCUGUUUCCCCGGUCAACGUUGAUAUGCUCCAA | |
| | CAGGGCAAAAACAACUGAGAUCGUUAUCCGCAAAGUGCCUACGCAAAGCCUAGUAAUGCUUUCGAAG | |
| | UCAUUUGGUUGGUCGCUCCGCUGUAACCCACAGUAGACCUGGCAGAUGAGGCUAGAAACUCCCCACU | |
| | GGCGACAGUGUUCUAGCCUGCGUGGCUGCCUGCACACCCUUUGGGUGUGAAGCCAAAUUACGGACAA | |
| | GGUGUGAAGAGCCCCGUGUGCUCACUUUGAGUCCUCCGGCCCCUGAAUGUGGCUAACCUUAACCCUG | |
| | CAGCUAGAGUGCACAAUCCAGUGUAUUUCUAGUCGUAAUGAGCAAUUGCGGGAUGGGACCAACUACU | |
| | UUGGGUGUCCGUGUUUCACUUUCUUUUUUUAUCUUUUAUUUGCUUAUGGUGACAAUAUAUUGAAUUA | |
| | UAUAUUGACACC | |
| 395 | UUAAAACUGGAUCUGGGUUGUUCCCACCCAGAUCACCUACAUGGUGUUGUACACUAUUAUUACGGUA | rhinovirusA16 |
| | AUCUUGUACGCCAGUUUUAUACUCCCUUUCCCAAAUGUAACUUAGAAGCAAAGCAAACUGAUCAAUA | |
| | GGGGACAAUCAUCCAGAUUGUCAAAGAUCAAGCACUUCUGUUUCCCCGGUCAAAGUUGAUAUGCUCC | |
| | ACAAGGGCAAAAACAACUGAGAUCGUUAUCCGCAAGAUGCCUACGCAAAGCCUAGUAAUACAUUGAA | |
| | AGAUACUUGGUUGGUCGCUCAGCUGUUAACCCAACAGUAGACCUGGUAGAUGAGGCUAGAGAUUCCC | |
| | CUCCGGCGACGGAGUUCUAGCCUGCGUGGCUGCCUGCACACCCACUGGGUGUGAAGCCAAGUAUUGG | |
| | ACAAGGUGUGAAGAGCCGCGUGUGCUCAUCUUGAGUCCUCCGGCCCCUGAAUGUGGCUAACCUUAAA | |
| | CCUGCAGCCAGUGCACACAAUCCAGUGUGUAGCUGGUCGUAAUGAGCAAUUGCGGGAUGGGACCAAC | |
| | UACUUUGGGUGUCCGUGUCCGUGUUUCACUUUUUUUCCUUUUAUUAUUGCUUAUGGUGACAAUAUAU | |
| | AUAGCAUAUAUAUAUUGUUAUC | |
| 396 | UUAAAACUGAAUCCAGGUUGUUCCCACCUGGAUUUCCCAUGUGGAGUAGUACACUGUUAUUCCGGUA | rhinovirusA18 |
| | ACUUUGUACGCCAGUUUUAUAACCCCUUCCCCUUAUGUAACUUAGAAGAUAAGCACAAAGACCAAUA | |
| | GGUGGUGGCUAUCCCAGUCACUAAAGGUCAAGCACUUCUGUUUCCCCGGUCAACGUUGAUAUGCUCC | |
| | ACCAGGGCAAAAACAACCGAGAUCGUUAUCCGCAAAGUGCCUACGCAAAGCCUAGUAGCACUUUGAA | |
| | UAGCAUGUGGUUGGUCGCUCCACUAUUCCCCAUAGUAGACCUGGCAGAUGAGGCUAGAAAUCCCCCA | |
| | CUGGCGACAGUGUUCUAGCCUGCGUGGCUGCCUGCGUGUCCUAACGGACACGAAGCCAUAUGUUUGA | |
| | CAAGGUGUGAAGAGCCCCGUGUGCUCACUUUGAGUCCUCCGGCCCCCGAAUGUGGCUAACCUUAACC | |
| | CUGCAGCUGGGGCAUACAAUCCAGUGUGAACUCAGUCGUAAUGAGCAAUUGCGGGAUGGGACCAACU | |
| | ACUUUGGGUGUCCGUGUUUCACUUUUUUUUAUAAUGCUUAUGGUGACAAUAUAUAAUAAUUUGUAUA | |
| | UUGGCACC | |
| 397 | UUAAAACUGGAUCUGGGUUGUUCCCACCCAGAUCAUCCACGUGAUGUUGUGCUCUAUUAUUCCGGUA | rhinovirusA19 |
| | AUUUUGCACGCCAGUUUUUCAUCCCUUCCCCCUUUGUAACUUAGAAGACUUAAACAAAUGACCAAUA | |
| | GGAAGCAACAAACCAGUUGCGAUAGGUCAAGCACUUCUGUUUCCCCGGUCAAUGUUGAUAUGCUCCA | |
| | ACGGGGCAAAAACAGCAGCGAUCGUUAUCCGCAAGGUGCCUACGCAAAGCCUAGUAGCGCUCUUGAA | |
| | AUUGUUUGGUUGGUCGCUCAAGUACAAACCCAGUACUAGACCUGGCAGAUGAGGCUAGAAAACCCCC | |
| | ACUGGCGACAGUGUUCUAGCCUGCGUGGCUGCCUGCACACCCAUUUGGGUGUGAAGCCAAACAACGG | |
| | ACAGGGUGUGAAGAGCCACGUGUGCUCAUCUUGAUUCCUCCGGCCCCUGAAUGCGGCUAACCUUAAA | |
| | CCUGCAGCCAUUGCUCACAAUCCAGUGAGUUGGUGGUCGUAAUGAGCAAUUGCGGGAUGGGACCGAC | |
| | UACUUUGGGUGUCCGUGUUUCACUUUUUACCUUGUUUUAUGCUUAUGGUGACAAUAUAUAGUAGUAU | |
| | AUAUUGACACC | |
| 398 | UUAAAACUGGAUCCAGGUUGCUCCCACCUGGAUCUCCUACAUGGAGUUGUACUCUAUUAUUCCGGUA | rhinovirusA20 |
| | AUUUUGUACGCCAGUUUUAUCUCCCCCUACCCCCUUACGUAACUUAGAAGUUUUACACACACCGACC | |
| | AAUAGGUGGUAUUUACCCAAAAUACCAAAGGUCAAGCACUUCUGUUACCCCGGUCAAAAUUGAUAUG | |
| | CUCCACCAGGGCAAAAACAAUUGCGAUCGUUAUCCGCAAAGCAACUACGCAAAGCCUAGUACCAUUU | |
| | UUGAUGAUGUGUGGUUGGUCGUUCCACUAUGAACCCGAUAGUAGACCUGGCAGAUGAGGCUAGAAAU | |
| | UCCCCACUGGCAACAGUGUUCUAGCCUGCGUGGCUGCCUGCGUACCCCCUGUGGGUACGAAGCCAUA | |
| | CAUUUGACAAGGUGUGAAGAGCCCCGUGUGCUUGCUUUGAGUCCUCCGGCCCCUGAAUGUGGCUAAC | |
| | CUUAACCCUGCAGCUAGUGUGUACAAUCCAGUACAUAGCUAGUCGUAAUGAGCAAUUGCGGGAUGGG | |
| | ACCAACUACUUUGGGUGUCCGUGUUUCUUGUUUUUCUUUCAUAUUUGCUUAUGGUGACAAUAUAUAU | |
| | AACACAUAUAUUGGCACC | |
| 399 | UUAAAACUGGGUCCAGGUUGUUCCCACCUGGAUCUCCUAUUGGGAGUUGUACUCUAUUAUUCCGGUA | rhinovirusA21 |
| | AUUUUGUACGCCAGUUUUAUCUCCCCUUCCCCAAUUGCAACUUAGAAGUUUAUCAAUAUGACCAAUA | |
| | GGCGGUAGUUAGCCAAACUACCAGAGGUCAAGCACUUCUGUUUCCCCGGUCAAAGUUGAUAUGCUCC | |
| | AACAGGGCAAAAACAACUGAGAUCGUUAUCCGCAAAGUGCCUACGCAAAGCCUAGUAACACCUUUGA | |
| | AGAUUUAUGGUUGGUCGUUCCGCUAUUUCCCAUAGUAGACCUGGCAGAUGAGGCUAGAAAUUCCCCA | |
| | CUGGCGACAGUGUUCUAGCCUGCGUGGCUGCCUGCGCACCCCUUGGGUGCGAAGCCAUACAUUGGAC | |
| | AAGGUGUGAAGAGCCCCGUGUGCUCACUUUGAGUCCUCCGGCCCCUGAAUGUGGCUAACCUUAACCC | |
| | UGCAGCUAGUGCAUGUAAUCCAACAUGUUGCUAGUCGUAAUGAGCAAUUGCGGGACGGGACCAACUA | |
| | CUUUGGGUGUCCGUGUUUCACUUUUUCCUUUUAAUAUUGCUUAUGGUGACAAUAUAUAUAAACAUAU | |
| | AUAUUGACACC | |
| 400 | UUAAAACUGGAUCUGGGUUGUUCCCACCCAGAUCACCCAAGUGGUGUAGUGCUCUGUUAUUUCGGUA | rhinovirusA22 |
| | ACCUUGCACGCCAGUUUUAUCACCCCUUCCCCACGUAACUUAGAAGCUUUUAAACACAGACCAAUAG | |
| | GGAGCGACAAACCAGUCGUGAAAGGUCAAGCACUUCUGUUUCCCCGGUCAAUGUUGAUAUGCUCCUC | |
| | CAGGGCAAAAACAACAGCGAUCGUUACCCGCAAGAUGCCUACGCAAAGCUUAGUAAUAUCUUCGAAG | |
| | AUACUUGGUUGGUCGCUCAUGUGCAACCCCAGCAUUAGACCUGGCAGAUGAGGCUAGAAAUUCCCCA | |
| | CUGGCGACAGUGUUCUAGCCUGCGUGGCUGCCUGCACACCCUAACGGGUGUGAAGCCCAGUAUUGGA | |
| | CAAGGUGUGAAGAGCCCCGUGUGCUCAUCUUGAGUCCUCCGGCCCCUGAAUGCGGCUAACCUUAACC | |
| | CUGCAGCCAUUGCUCACAAUCCAGUGAGUCUAUGGUCGUAAUGAGCAAUUGCgGGAUGGGACCGACU | |
| | ACUUUGGGUGUCCGUGUUUCUCUUUUUUCCUUAUAUUUGAGCUUAUGGUGACAAUAUAUAGUGAUAU | |
| | AUAUUGGCAUC | |
| 401 | CCAAAGUAGUUGGUCCCGUCCCGCAUGCAACUUAGAAGCUUUGCACAAAGACCAAUAGCCAGUAAUC | rhinovirusA23 |
| | AUCCAGAUUACUUAAGGUCAAGCACUUCUGUUUCCCCGGUCAAAGUUGAUAUGCUCCACCAGGGCAA | |
| | AAACAACUGCGAUCGUUAUCCGCAAGGCGCCUACACAAAGCUUAGUAGCAUUCUGAAAGUUGUCUGG | |
| | UUGGUCGUUCCACCAUUCCCCCUGGUAGACCUGGCAGAUGAGGCUAGAAACUCCCCACUGGCGACAG | |
| | UGUUCUAGCCUGCGUGGCUGCCUGCACACCCUCUGGGUGUGAAGCCAUACGAUGGACAGGGUGUGAA | |
| | GAGCCCCGUGUGCUCGCUUUGAGUCCUCCGGCCCCUGAAUGUGGCUAACCUUAACCCUGCAGCUAGA | |
| | GCACACAAUCCAGUGUGUUUCUAGUCGUAAUGAGCAAUUGCGGGACGGGACCAACUACUUUGGGUGU | |
| | CCGUGUUUCCUUGUUCUCUUUGAAUAUCUGCUUAUGGUGACAAUAUAUACGUAUAUAUUGACACC | |
| 402 | UUAAAACUGGAUCUAGGUUGUUCCCACCUAAAUCUCCUACAUGGAGUUGUACUCUAUUAUUCCGGUA | rhinovirusA24 |
| | AUUUUGUACGCCAGUUUUAUCCUCCCCUCCCCUUAAAGUAACUUAGAAGUUGGACAUAUCGACCAAU | |
| | AGGUAACAUUUAUCCAAAAUGUUAAAGGUCAAGCACUUCUGUUUCCCCGGUCAAGGUUGAUGUGCUC | |
| | UAACAGGGCAAAAACAGCUGCGAUCGUUAUCCGCAAAGCAACUACGCAAAGCCUAGUAGCAUUUUUU | |
| | GAGAGUAUGUGGUUGGUCGCUCCACUAUAAACCCAAUAGUAGACCUGGCAGAUGAGGCUAGAAAUUC | |
| | CCCACUGGUAACAGUGUUCUAGCCUGCGUGGCUGCCUGCGUGCUCCUUGUGAGCACGAAGCCAUAUA | |
| | UUUGACAAGGUGUGAAGAGCCCCGUGUGCUUGCUUUGAGUCCUCCGGCCCCUGAAUGUGGCUAACCU | |
| | UAACCCUGCAGCUAGCGUGUACAAUCCAGUACAAAGCUAGUCGUAAUGAGCAAUUGCGGGAUGGGAC | |
| | CAACUACUUUGGGUGUCCGUGUUUCCUGUUUUUCUUUUAUAUUUGCUUAUGGUGACAAUAUAUAUAU | |
| | AACAUAUAUAUUGGCAUC | |
| 403 | UUAAAACUGGAUCCAGGUUGUUCCCACCUGGAUCUCCUACAUGGAGUUGUACUCUAUUAUUCCGGUA | rhinovirusA25 |
| | AUCUUGUACGCCAGUUUUAUACCCCCUAACCCCUUGUAACUUAGAAGUUAAGCACAUAGACCAAUAG | |
| | GCGGUGGCCACCCAGGUCGCUAACGGUCAAGCACUUCUGUUUUCCCGGUUAAGAUUGAUACGCUCCA | |
| | ACAGGGCGAAAACAGUCUGAAUCGUUAACCGCAAAGUGACUACGCAAAGCUUAGUAAUGCCUUGAAG | |
| | AAUUUAUGACUGGUCGUUCCGCUAUAACCCCUAGUAGACCUGGCAGAUGAGGCUAGAAAUACCCCAC | |
| | UGGUAACAGUGUUCUAGCCUGCGUGGCUGCCUGCACACCCUUUCGGGUGUGAAGCCAUAUAUUUGAC | |
| | AAGGUGUGAAGAGCCCCGUGUGCUCACUUUGAGUCCUCCGGCCCCUGAAUGUGGCUAACCUUAACCC | |
| | UGCAGCUAGUGUAUGUAAUCCAACAUAUGGCUAGUCGUAAUGAGCAAUUGCGGGAUGGGACCAACUA | |
| | CUUUGGGUGUCCGUGUUUCACUUUUUCCUUUUAUUUUGCUUAUGGUGACAAUAUAUAUAUUAUAUAU | |
| | AUUGGCAUC | |
| 404 | UUAAAACUGGGUCUAGGUUGUUCCCGCCUAGAUUACCCAUGUGGUGUAGUACUCUAUUAUUACGGUA | rhinovirusA28 |
| | AUUUUGUACGCCAGUUUUAUAUCCCCCCCCCCCUGUUUGUAACUUAGAAGUUUUGAACUUGGAGACC | |
| | AAUAGGCGGUGAUUAUCCAGAUCAUCAACGGUCAAGCACUUCUGUUUCCCCCGGUUAGAUUGAUACG | |
| | CUCCAACAGGGCGAAAACAACUCUAAUCGUUAUCCGCAAGAUGCCUACGCAAAGCUUAGUACCACCU | |
| | UCGAUUCUAUUUGGUUGGUCGUUCAGCUACAAAACCCAGUAGUAGACCUGGCAGAUGAGGCUAGAAA | |
| | UACCCCACUGGCGACAGUGUUCUAGCCUGCGUGGCUGCCUGCACACCCUAUGGGUGUGAAGCCAAUU | |
| | AGUUGACAAGGUGUGAAGAGCCCCGUGUGCUCAUCUUGAUUCCUCCGGCCCCUGAAUGUGGCUAACC | |
| | UUAAACCUGCAGCCAAUGUACACAAUCCAGUGUAUUAUUGGUCGUAAUGAGUAAUUGCGGGAUGGGA | |
| | CCAACUACUUUGGGUGUCCGUGUUUCUCUUUUUUACCUUUAUUAUUGCUUAUGGUGACAGUAUAUGG | |
| | UAUAUAUAUAUUGUCACU | |
| 405 | UUAAAACUGGAUCUGGGUUGUUCCCACCUGGAUCUCCUACAUGGAGUUGUACUCUAUUAUUCCGGUA | rhinovirusA29 |
| | AUCUUGUACGCCAGUUUUAUACCCCUUUCCCUUUUGUAACUUAGAAGUUAAACACACAGACCAAUAG | |
| | GUGAUGGCUAUCCAAGUCAUUAACGGUCAAGCACUUCUGUUUCCCCGGUUUAAAUUGAUACGCUCCA | |
| | CCAGGGCGAAAACAAUUUAAAUCGUUAUCCGCAAAAUGACUACGCAAAGCCUAGUAACAUCUUGAAG | |
| | AACUUAUGGUUGGUCGUUCCACUAAACCCCAUAGUAGACCUGGCAGAUGAGGCUAGAAAUACCCCAC | |
| | UGGAGACAGUGUUCUAGCCUGCGUGGCUGCCUGCACACCCUAUGGGUGUGAAGCCAUAUUUUUGACA | |
| | AGGUGUGAAGAGCCCCGUGUGCUCAUUUUGAGUCCUCCGGCCCCUGAAUGUGGCUAACCUUAACCCU | |
| | GCAGCUAGUGCAUGCAAUCCAGCAUGUUGCUAGUCGUAAUGAGCAAUUGCGGGAUGGGACCAACUAC | |
| | UUUGGGUGUCCGUGUUUCACUUUUUCCUUUUAUUUGCUUAUGGUGACAAUAUAUAUAGUACAUAUAU | |
| | AUUGGCAUC | |
| 406 | UUAAAACUGGAUCCAGGUUGUCCCCACCUGGAUCUCCCACAGGGAGUGGUACUCUGUUAUUACGGUA | rhinovirusA30 |
| | ACUUUGUAUGCCAGUUUUAUCCUCCCCACCCCUAUGUAACUUAGAAGUUUUACAUAAAGACCAAUAG | |
| | GUAGUAAUCAUCCAGAUUACUUAAGGUCAAGCACUUCUGUUUCCCCGGUUGAUGUUGAUAUGCUCCA | |
| | ACAGGGCAAAAACAACAGAAAUCGUUACCCGCAAAGUGCCUACGCAAAGCCUAGUAGCACUUUAGAA | |
| | GUUGUCUGGCUGGUCGCUCCGCCAUUUCCCCUGGUAGACCUGGCAGAUGAGGCUAGAAAUACCCCAC | |
| | UGGCGACAGUGUUCUAGCCUGCGUGGCUGCCUGCACACCCUAUGGGUGUGAAGCCAUACAAUGGACA | |
| | AGGUGUGAAGAGCCCCGUGUGCUCACUUUGAGUCCUCCGGCCCCUGAAUGUGGCUAACCUUAACCCU | |
| | GCAGCUAGGGCACACAAUCCAGUGUGUAUCUAGUCGUAAUGAGCAAUUGCGGGACGGGACCAACUAC | |
| | UUUGGGUGUCCGUGUUUCUCUUUUAUCCUUAUGUUUGCUUAUGGUGACAAUAUAUGAAUAUAUAUUG | |
| | ACACC | |
| 407 | UUAAAACUGGGUGCGAGUUGUUCCCACUCGCACUUCCUACGUGGAGUAGUACACUAUUAUUACGGUA | rhinovirusA31 |
| | AUUUUGUACGCCAGUUUUAUAACCCUUCCCCUUUUGUAACUUAGAAGUUUGAACUUGAGACCAAUAG | |
| | GUGGUGAUCAACCAGAUCGCCAAAGGUCAAGCACUUCUGUUUCCCCGGUCAACAUUGAUAUGCUCCA | |
| | CCAGGGCAAAAACAAUUGCGAUCGUUACCCGCAAAGUGCCUACGCAAAGCUUAGUAAUAUCUUGAAC | |
| | GAUAUAUGGUUGGUCGCUCCACUAAUCCCCAUAGUAGACCUGGCAGAUGAGGCUGGAAACUCCCCAC | |
| | UGGCGACAGUGUUCCAGCCUGCGUGGCUGCCUGCGCACUCUCACGAGUGCGAAGCCAUAUAUUUGAC | |
| | AAGGUGUGAAGAGCCCCGUGUGCUCACUUUGAGUCCUCCGGCCCCUGAAUGUGGCUAACCUUAACCC | |
| | UGCAGCUAGUGCAUACAAACCAGUAUGUGGCUAGUCGUAAUGAGCAAUUGCGGGACGGGACCAACUA | |
| | CUUUGGGUGUCCGUGUUUCACUUUUUCUUUUAUGGUUUGCUUAUGGUGACAGUAUAUAGUGUAUAUA | |
| | UUGUUACC | |
| 408 | UUAAAACUGGAUCCGGGUUGUUCCCACCCGGAUCUCCCACGUGGAGUUGUACACUAUUAUUACGGUA | rhinovirusA32 |
| | AUUUUGUACGCCAGUUUUAUAUCCCUUACCCCAAGUAACUUAGAAGAUAAACACACAGACCAAUAGG | |
| | AGGUGGUCAUCCAGAUCACUAAGGGUCAAGCACUUCUGUUUCCCCGGUCAAGGUUGAUAUGCUCCAA | |
| | CAGGGCAAAAACAGCUGAGAUCAUUAUCCGCAAAGUGCCUACGCAAAGCCUAGUAACUACCUCGAAA | |
| | GUACUUGACUGGUCGCUCCGCCAUACCCCAUGGUAGACCUGGCAGAUGAGGCUAGAAAUCCCCCACU | |
| | GGUGACAGUGUUCUAGCCUGCGUGGCUGCCUGCACACCCUUUGGGUGUGAAGCCAAGUAAUGGACAG | |
| | GGUGUGAAGAGUCGAGUGUGCUCACUUUGAGUCCUCCGGCCCCUGAAUGUGGCUAACCCUAACCCUG | |
| | CAGCUAGGGCACACAAUCCAGUGUGUAUCUAGUCGUAAUGAGCAAUUGCGGGAUGGGACCAACUACU | |
| | UUGGGUGUCCGUGUUUCUUGUUUUUCAUUUAUAAUUGCUUAUGGUGACAAUAUAUAGUAUUAUAUAU | |
| | UGGCGUC | |
| 409 | UUAAAACUGGAUCUAGGUUGUUCCCACCUAGAUCUCCUACACGGAGUUGUACUCUGUUAUUCCGGUA | rhinovirusA33 |
| | ACUUUGUACGCCAGUUUUAUACCCCCUUCCCCCUUUGUAACUUAGAAGCUGAACAUACUGACCAAUA | |
| | GGCGGCAUUCAACCAGAAUGUCAAAGGUCAAGCACUUCUGUUUCCCCGGUUAAAGCUGAUAUGCUCC | |
| | AACAGGGCAAAAACAGCCUUAAUCGUUAUCCGCAAGACAACUACGCAAAGCCUAGUAUCAUCUUUGA | |
| | AAGUAUGUGGUUGGUCGCUCCGCUAUAAACUCAAUAGCAGACCUGGCAGAUGAGGCUAGAAACUCCC | |
| | CACUGGCGACAGUGUUCUAGCCUGCGCGGCUGCCUGCGUGCCCUAUACGGGUACGAAGCCAUAUAUU | |
| | UGACAAGGUGUGAAGAGCCCCGUGUGCUUGUCUUGAGUCCUCCGGCCCCUGAAUGUGGCUAACCUUA | |
| | ACCCUGCAGCUGGUAUGCACAAUCCAGUACAUGGCCAGUCGUAAUGAGCAAUUGCGGGAUGGGACCA | |
| | ACUACUUUGGGUGUCCGUGUUUCCUAUUUUACCUUUAUAUUUGCUUAUGGUGACAAUAUAUAUAUAA | |
| | CAUAUAUAUUGGCAUC | |
| 410 | UUAAAACUGGGUCCAGGUUGUUCCCACCUGGACUUCCCACGUGGAGUUGUGCACUGUUAUUCCGGUA | rhinovirusA34 |
| | ACUUUACACGCCAGUUUUAUCUCCCUUCCCAAAUGUAACUUAGAAGAUGUACACAGCGACCAAUAGG | |
| | UGGCAACUAGCCAAGUUGCUUAAGGUCAAGCACUUCUGUCCCCCCGGUCAAUGUUGAUACGCUCUCC | |
| | CAGGGCGAAAACAACUGAGAUCGUUAUCCGCAAGGCGCCUACACAAAACCUAGUAACAUCUUGAAUG | |
| | GUAUGUGGCUGGUCGUUCCGCUAAACCCCAUAGUAGACCUGGCAGAUGAGGCUAGAAAUUCCCCACU | |
| | GGCGACAGUGUUCUAGCCUGCGUGGCUGCCUGCGUGUCCUUACGGACACGAAGCCAUAUACUUGACA | |
| | AGGUGUGAAGAGUCCCGUGUGCUCGUCUUGAGUCCUCCGGCCCCUGAAUGUGGCUAACCUUAACCCU | |
| | GCAGCCAGUGCGCACAAACCAGUGUGUAGCUGGUCGUAAUGAGUAAUUGCGGGAUGGGACCAACUAC | |
| | UUUGGGUGUCCGUGUUUCACUUUUUACCUUUUAUUAUGCUUAUGGUGACAAUAUAUAGAUGCAUAUA | |
| | UUGACACC | |
| 411 | UUAAAACUGGGAGUGGGUUGUUCCCACUCACUCCACCCAUGCGGUGUUGUACACUGUUAUUACGGUA | rhinovirusA36 |
| | ACCUUGUACGCCAGUUUUGCCACCCUUCCCCAUUAUGUAACUUAGAAGUUUGUACAACAAGACCAAU | |
| | AGGUGAUAAUUAUCCAGAUUAUCAAAGGUCAAGCACUUCUGUUUCCCCGGUCGAUGAGGAUAUGCUU | |
| | UACCCAAGGCAAAAACCUUAAAGAUCGUUAUCCCCACACUGCCUACACAGAGCCUAGUACCACUUUU | |
| | GAUGUAAUUAGGUUGGUCGCUCCCUGCAAACCCAGCAGUAGACCUGGCAGAUGAGGCUGGACACUCC | |
| | CCACUGGUGACAGUGGUCCAGCCUGCGUGGCUGCCUGCUCACCCUAUUGGGUGAGAAGCCUACUUAU | |
| | UGACAAGAUGUGAAGAGCCGCGUGUGCUCAGUGUGCUUCCUCCGGCCCCUGAAUGUGGCUAACCUUA | |
| | ACCCUGCAGCCGCUGCCCAUAAUCCAAUGGGUUUACGGUCGCAACGCGCAAGUGCGGGAUGGGACCA | |
| | ACUACUUUGGGUGUCCGUGUUUCCUAUUUUUCUUUUAUUUGCAUUUUAUGGUGACAAUUUAUAGUAU | |
| | AUAGAUUGUCGUC | |
| 412 | UUAAAACUGGAUCUGGGUUGUUCCCACUCAGAUCUCCCAUGCGGAGUUGUACUCUAUUAUUCCGGUA | rhinovirusA38 |
| | AUCUUGUACGCCAGUUUUUACACCCUCCCCCCUUGUAACUUAGAAGAAAGUACAUAACGACCAAUAG | |
| | GUGGCAAUUAACCAAAUUGUCAACGGUCAAGCACUUCUGUUUCCCCGGUCAAGAUUGAUACGCUCCA | |
| | ACAGGGCGAAAACAAUUGAGAUCGUUAUCCGCAAAGUGCCCACGCAAAGCCUAGUAACACUUUGAAA | |
| | GGUCUAUGGUUGGUCGUUCAGCUAAACCCCAUAGUAGACCUGGCAGAUGAGGCUAGAAAUUCCCCAC | |
| | UGGCGACAGUGUUCUAGCCUGCGUGGCUGCCUGCACACCCUCACGGGUGUGAAGCCAUAUACUCGAC | |
| | AAGGUGUGAAGAGCCCCGUGUGCUCACUUUGAGUCCUCCGGCCCCUGAAUGUGGCUAACCUUAACCC | |
| | CGCAGCUAGUGCAUACAAACCAGUAUGUGGCUAGUCGUAAUGAGCAAUUGUGGGAUGGGACCAACUA | |
| | CUUUGGGUGUCCGUGUUUCACUUUUUUCCUUUUGUUUGCUUAUGGUGACAAUAUAUAUAGUAUAUAU | |
| | UGACACC | |
| 413 | UUAAAACUGGAUCUGGGUUGUUCCCACCUAGAUCACCUACAGGGUGUAGUACACUGUUAUUACGGUA | rhinovirusA39 |
| | ACUUUGUACGCCAGUUUUAUAUCCCCCCCCCCAACGUAACUUAGAAGCAAGCACAAACGUCCAAUAG | |
| | GCGGUGAUCAUCCAAAUCACUUACGGACAAGCACUUCUAGUCUCCCCGGCAACUGCUGAUAUGCUCC | |
| | AACAGGGCAAAAACAGCGGGAGUCGUUAUCCGCAAAAUGCCUACGCAAAGCCUAGUAUCACCCUAGA | |
| | AGGUGUGUGGUUGGUCGCUCAGCCGUGAACCCGACGGUAGACCUGGCAGAUGAGGCUAGAAAUACCC | |
| | CACUGGCGACAGUGUUCUAGCCUGCGUGGCUGCCUGCACACCCUCACGGGUGUGAAGCCAUUCAUUG | |
| | GACAGGGUGAGAAGAGCCCAGUGUGCUCAUUUUGAGUCCUCCGGCCCCUGAAUGUGGCUAACCUUAA | |
| | ACCUGCAGCCAAUGCACACAAUCCAGUGUGUAUUUGGUCGUAACGAGCAAUUGCGGGAUGGGACCAA | |
| | CUACUUUGGGUGUCCGUGUUUCUUUUUAUUCUUAUCAUUUUGCCUAUGGUGACAUAUAUACUGUUAU | |
| | AUAUUGUUAUC | |
| 414 | UUAAAACUGGAUCCAGGUUGUUCCCACCUGGAUUUCCUACAUGGAGUUGUACUCUGUUAUUCCGGUA | rhinovirusA40 |
| | ACCUUGUACGCCAGUUUUAUAUCCCCCCCCAUUGUAACUUAGAAGUUAUGCAUACAGACCAAUAGGU | |
| | GGUGAUCCUCCAGAUCACUAACGGUCAAGCACUUCUGUUUCCCCGGUUUGAAUUGAUACGCUCCAAC | |
| | AGGGCGAAAACAGUUUAAAUCGUUAUCCGCAAAGUGCCUACGCAAAGCCUAGUAGCACUUUAGAAAA | |
| | UCUAUGGUUGGUCGUUCAGCUAUACCCCCUAGUGGACCUGGCAGAUGAGGCUAGAAGUACCCCACUG | |
| | GCGACAGUGUUCUAGCCUGCGUGGCUGCCUGCACACCCUUAUGGGUGUGAAGCCAUAUAUUUGACAA | |
| | GGUGUGAAGAGCCCCGUGUGCUCACUUUGAGUCCUCCGGCCCCUGAAUGUGGCUAACCUUAACCCUG | |
| | CAGCUAGUGCAUGUAAUCCAACAUGUUGCUAGUCGUAAUGAGCAAUUGCGGGAUGGGACCAACUACU | |
| | UUGGGUGUCCGUGUUUCACUUUUUCCUUUUAAUUUGCUUAUGGUGACAAUAUAUAUAUAACAUAUAU | |
| | AUUGGCAUC | |
| 415 | UUAAAACUGGGUUUGGGUUGAUCCCACCCAAACCACCCACGUGGUGUAGUACACUGUUAUCUCGGUA | rhinovirusA41 |
| | ACUUUGUACGCCAGUUUUAUCUCCCCCUCCCCCCUUUGUAACUUAGAAGUCAUGCACAAUGACCAAU | |
| | AGCAGGCAAUCAUCCAGAUUGUCAUCGGUCAAGUACUUCUGUUUCCCCGGUUCAGAUUGAUAUGCUC | |
| | CAACAGGGCAAAAACAACCUGGAUCGUUAUCCGCAAGAUGCCUACGCAGAACCUAGUAUCACACUGG | |
| | AAGAUUCUCGGUUGGUCGCUCAGUUACAACCCCAGUAACAGACCUGGCAGAUGAGGCUAGAAAUUCC | |
| | CCACUGGCGACAGUGUUCUAGCCUGCGUGGCUGCCUGCACACCCGUUUGGGUGUGAAGCCUAGAAUU | |
| | GGACAGGGUGCGAAGAGUCGAGUGUGCUCAUCUUGAGUCCUCCGGCCCCUGAAUGCGGCUAACCCUA | |
| | ACCCUGCAGCCAUUGCCCACAAACCAGUGGGUCUAUGGUUGUAAUGAGCAAUUGCGGGAUGGGACCG | |
| | ACUACUUUGGGUGUCCGUGUUUCACUUUUUACUUAUUAUUUGCUUAUGGUGACAAUAUAUAGAUACA | |
| | UAUAUUGACACC | |
| 416 | UUAAAACUGGGUCCAGGUUGCUCCCACCUGGAUCACUCACUGAGUGUUGUAUACUGUUAUUACGGUA | rhinovirusA43 |
| | ACUUUAUACGCCAGUUUUAUCUCCCCUUACCCCAAUGUAACUUAGAAGUUUGUACAUACCGACCAAU | |
| | AGUGAGCAAUCAUCCAGAUUGUUUAAGGUCAAGCACUUCUGUUACCCCGGUCAGCGUUGAUAUGCUC | |
| | CAACAGGGCAAAAACAACAAUGAUCGUUAUCCACAAGAUGCCUACACAGAGCCUAGUAACAUUCUAG | |
| | AAAAAUUCUGGUUGGUCGCUCAAUUGCAAACCCAGCAAUAGACCUGGCAGAUGAGGCUGGACAUUCC | |
| | CCACUGGCGACAGUGGUCCAGCCUGCGUGGCUGCCUGCACACUCCUUUGGAGUGUGAAGCCAUAAUU | |
| | UGGACAGGGUGCGAAGAGCCGCGUGUGCUCAUCUUGAGUCCUCCGGCCCCUGAAUGCGGCUAACCUU | |
| | AAACCUGCAGCCAUUGUUUGCAAUCCAGCAGAUaUAUGGUCGUAAUGAGCAAUUGCGGGAUGGGACC | |
| | GACUACUUUGGGUGUCCGUGUUUCCUUUAUUUCCAUUUAUAUGCUUAUGGUGACAAUAUAUGAUAUA | |
| | UAUUGACACC | |
| 417 | UUAAAACUGGGUCGUGGUUGUUCCCACCACGACUAUCUACGGAUGUAGUGCUCUUGUAUUCCGGUAC | rhinovirusA45 |
| | GCUUGCACGCCAGUUUUGUCACCCCCCCCUACAUUGUAACUUAGAAGAUUAACACAAAGACCAAUAG | |
| | GCGGCAAUGAACCACAUUGUCAACGGUCAAGCACUUCUGUUUCCCCGGUCAAUCUAGAUAUGCUUUA | |
| | CCAAAGGCAAAAACUGGAGCGAUCGUUAUCCGCAAGGUGCCUACGGGAAACCCAGUAGCAUUCUUUA | |
| | UUUGAUCUGGUUGGUUGCUCAGCCGUUAAACCCAAACGGUAAACCUGGCAGAUGAGGCUGGAAGAUC | |
| | CCCACCAGCGAUGGUGUUCCAGCCUGCGUGGCUGCCUGCACACCCGAAAGGGUGUGAAGCCUUUUCA | |
| | AAGACAGGGUGCGAAGAGUCUACUGUGCUCACCUUGAUUCCUCCGGCCCCUGAAUGUGGCUAAUCCU | |
| | AACCCCGUAGCUGUGGUGUGCAAUCCAGCACAUUCGCAGUCGUAAUGGGUAACUGCGGGAUGGAACC | |
| | AACUACUUUGGGUGUCCGUGUUUCUCUUUUUCCUUUUAUAUAUGCUUAUGGUGACAAUUAUACGGAU | |
| | AUAGUUGUCAUC | |
| 418 | UUAAAACUGGAUCUGGGUUGUUCCCACCCAGAUUUCCCAUGUGGAGUAGUACUCUAUUAUUCCGGUA | rhinovirusA46 |
| | AUUUUGUACGCCAGUUUUAUAUUCCCUACCCCGAGUAACUUAGAAGAUAAGCACACAAGGCCAAUAG | |
| | UGGGUGAUUAGCCAAAUCAUCAAAGGUCAAGCACUUCUGUUUCCCCCGGUCAAGGUUGAUAUGCUCU | |
| | ACCAGGGCAAAAACAACUGAGAUCGUUAUCCGCAAAAUGCCUACACAAAGCCUAGUAUCAUCUUGGA | |
| | AGGCACAGAGUUGGUCGCUCCACUAUUCCCCAUAGUAGACCUGGCAGAUGAGGCUAGAAAAUCCCCA | |
| | CUGGCGACAGUGUUCUAGCCUGCGUGGCUGCCUGCGUGCCUUGCGAGGCACGAAGCCCUGUGUUUGA | |
| | CAAGGUGUGAAGAGCCCCGUGUGCUCAUUUUGAUUCCUCCGGCCCCUGAAUGUGGCUAACCUUAACC | |
| | CUGCAGCUAGUGCACACAAACCAGUGUGUAUCUAGUCGUAAUGAGUAAUUGCGGGACGGGACCAACU | |
| | ACUUUGGGUGUCCGUGUUUCACUUUUUACCUUUUAAUUGCUUAUGGUGACAAUAUAUAUAAAUAUAU | |
| | AUUGACACC | |
| 419 | UUAAAACUGGGUGUGGGUUGUUCCCACCCACACCUCCCACGUGGAGUAGUACACUGUUAUCACGGUA | rhinovirusA47 |
| | AUUUUGUACGCCAGUUUUAUAACCCCUUUCCCUUUUGUAACUUAGAAGUUUGAACUUGAGACCaAUA | |
| | GGCGGUGGUCAACCAGAUCACUAAAGGUCAAGCACUUCUGUUUCCCCGGUCAACGUUGAUAUGCUCC | |
| | AGCAGGGCAAAAACAGCUGCGAUCGUUAUCCGCAAAGUGCCUACGCAAAGCUUAGUAACAUCUUGAA | |
| | UGAUAUAUGGUUGGUCGUUCCACUAAACCCCAUAGUAGACCUGGCAGAUGAGGCUGGAAAUUCCCCA | |
| | CUGGCGACAGUGUUCCAGCCUGCGUGGCUGCCUGCGCACUCUUAUGAGUGCGAAGCCAUAUAUUUGA | |
| | CAAGGUGUGAAGAGCCCCGUGUGCUCACUUUGAGUCCUCCGGCCCCUGAAUGUGGCUAACCUUAACC | |
| | CUGCAGCUAGUGCAUACAAUCCAGUGUGUAGCUAGUCGUAAUGAGCAAUUGCGGGAUGGGACCAACU | |
| | ACUUUGGGUGUCCGUGUUUCACUUUUCCUUUUAUGAUUUGCUUAUGGUGACAGUAUAUAGUUUAUAU | |
| | AUUGUUGCC | |
| 420 | UUAAAACUGGAUCCAGGUUGUUCCCACCUGGAUCUCCCAAAGGGAGUGGAACUCUGUUAUUACGGUA | rhinovirusA49 |
| | ACUUUGUACGCCAGUUUUAUCCUCCCCCACCCCAUGUAACUUAGAAGCUUUUCACAAAGACCAAUAG | |
| | UUGGUAAUCAUCCAGAUUACUUAAGGUCAAGCACUUCUGUUUCCCCGGUCAACGUUGAUAUGCUCUA | |
| | ACAGGGCAAAAACAACUGCGAUCGUUAUCCGCAAAGCGCCUACGCAAAGCCUAGUAACACCUUUGAA | |
| | AUUGUUUGGUUGGUCGUUCCGCCAUUUUCCCUGGUAGACCUGGCAGAUGAGGCUAGAAACACCCCAC | |
| | UGGCGACAGUGUUCUAGCCUGCGUGGCUGCCUGCACACCCUGUGGGUGUGAAGCCAAAUAAUGGACA | |
| | AGGUGUGAAGAGCCCCGUGUGCUCGCUUUGAGUCCUCCGGCCCCUGAAUGCGGCUAACCUUAACCCU | |
| | GCAGCUAGAGCACACAAGCCAGUGUGUAUCUAGUCGUAAUGAGUAAUUGCGGGACGGGACCGACUAC | |
| | UUUGGGUGUCCGUGUUUCACUUUUUACUUAUAAUUGCUUAUGGUGACAAUAUAUAUAUAGUAUAUAU | |
| | AUUGACACC | |
| 421 | UUAAAACUGGGUCCAGGUUGUUCCCACCUGGAUCUCCUAAUGGGAGUUGUACUCUGUUAUUCCGGUA | rhinovirusA50 |
| | ACUUUGUAUGCCAGUUUUAUCUCCCUACCCCCAAUGUAACUUAGAAGAUGUACACAACGACCAAUAG | |
| | GUGAUGAUCAACCAGAUUAUCAAAGGUCAAGCACUUCUGUUUUCCCGGUUAGAGUUGAUAUGCUCCA | |
| | ACAGGGCAAAAACAACUUUAAUCGUUAUCCGCAAAGCGCCUACGCAAAGCCUAGUAACAGCUUCGAA | |
| | GAUUUAUGGUUGGUCGUUCCGCUGUUCCCCACAGUAGACCUGGCAGAUGAGGCUAGAGAUUCCCCAC | |
| | UGGUAACAGUGUUCUAGCCUGCGUGGCUGCCUGCGCACCCUUCGGGUGCGAAGCCAUAUUUCGGACA | |
| | AGGUGCGAAGAGCCCCGUGUGCUCGCUUUGAGUCCUCCGGCCCCUGAAUGUGGCUAACCUUAACCCU | |
| | GCAGCUAGUGCAUACAAACCAGUAUGUGGCUAGUCGUAAUGAGCAAUUGCGGGAUGGGACCAACUAC | |
| | UUUGGGUGUCCGUGUUUCUCUUUUUCCUUUUAUUAUUGCUUAUGGUGACAAUAUAUAGUUUAUAUAU | |
| | UGGCACC | |
| 422 | UUAAAACUGGGUGUGGGUUGUACCCACCCACACUGUCUACGGACGUAGUGCUCUCAUAUUACGGUAU | rhinovirusA51 |
| | GUUUGCACGCCAGUUUUUACCCCCCCCCCAAAUUUGUAACAUUAGAAGUCUGCACUCUUAACCUGGU | |
| | CAAUAGGAGGUAGCAAUCCAUGCUAUCUAAGACCAAGCACUUCUGUUACCCCGGUAUGCGGAGAAUA | |
| | GACUCCAAAAGGGUUGAAGCUCCAAAUAUCGUUAUCCGCACAAUUACUACACAAAGCCUAGUAAUAU | |
| | CUUGAUAAGCUCUUUGGUUGGUCGUUCAGCUGUUACCCCCAGUAGACUUGGCAGAUGAGGCUGGGUG | |
| | UUCCCCACUGGCGACAGUGACCCAGCCUGCGUGGCUGCCUACACACCUGGAGACAGGUGUGAAGCCA | |
| | AAAGUGGACAAGGUGUGAAGAGCCCCGUGUGCUAAUUGUGAUUCCUCCGGCCCCUGAAUGCGGCUAA | |
| | UCCAACCCCACAGCCAAUGCAUGUAAACCAACAUGUAUUUGGUCGUAACGGGUAACUGUGGGACGGA | |
| | ACCGACUACUUUGGGUGUCCGUGUUUCCUUUUAUUUUUaUUGCUUAUGGUGACAAGUAUUGGUAUAU | |
| | AUUUGUCAUC | |
| 423 | UUAAAACUGGAUCUGGGUUGCUCCCACCCAGAUUACCCAAGUGGUGUAGUACUCUGUUAUUACGGUA | rhinovirusA53 |
| | GCUUUGUACGCCAGUUUUAUCACCCCUCCCCACAAUGUAACUUAGAAGUUUUACACUUACAGACCAA | |
| | UAGGAUGUGAUUAUCCAAAUCACUAAAGGUCAAGCACUUCUGUUUCCCCGGUUAGAUUUGAUACGCU | |
| | CCAACAGGGCGAAAACAGAUCUGAUCGUUAUCCGCAAGAUGCCUACGCAAAGCUUAGUAGCGCUCUG | |
| | AAUGCUUUUUGGCUGGUUGUUCCACUACAAACCCAGUAGUAAACCUGGCAGAUGAGGCUAGAAGUUC | |
| | CCCACUGGCGACAGUGUUCUAGCCUGCGUGGCUGCCUGCACACCCUAUGGGUGUGAAGCCAAUCAGU | |
| | UGACAGGGUGUGAAGAGCCCCGUGUGCUCAUCUUGAGUCCUCCGGCCCCUGAAUGUGGCUAACCUUA | |
| | AACCCGCAGCCAAGGUGUGCAAACCAGCACAUUCUUGGUCGUAACGAGUAAUUGUGGGAUGGGACCA | |
| | ACUACUUUGGGUGUCCGUGUUUCUUUUUAUUCUUUUACAAUGCCUAUGGUGACAAUAUAUAGUAUAU | |
| | AUUGUCAUU | |
| 424 | UUAAAACUGGGUCUAGGUUGUUCCCACCUAGACCUCCUACAUGGAGUUGUACUCUAUUAUUUCGGUA | rhinovirusA54 |
| | AUUUUGUACGCCAGUUUUAUAUCCCCUUUCCCCACGUAACUUAGAAGUUUAACACACUGACCAAUAG | |
| | GUGGCAAUUAACCAAAUUGCUAAAGGUCAAGCACUUCUGUUUCCCCGGUCAAGAUUGAUACGCUCUA | |
| | CCAGGGCGAAAACAAUUGAGAUCGUUAcCCGCAAAGUGCCUACGCGAAGCCUAGUAACAUCUUGAAG | |
| | AAUUUAUGGUUGGUCGCUCCACUAUUCCCCAUAGUAGACCUGGCAGAUGAGGCUAGAAAUUCCCCAC | |
| | UGGCGACAGUGUUCUAGCCUGCGUGGCUGCCUGCACACCCUUACGGGUGUGAAGCCAUAUAUUUGAC | |
| | AAGGUGUGAAGAGCCUAGUGUGCUCACUUUGAGUCCUCCGGCCCCUGAAUGUGGCUAACCUUAAACC | |
| | CGUAGCUAGUGCAUACAAUCCAGUGUGUGGCUAGUCGUAACGAGCAAUUACGGGAUGGGACCAACUA | |
| | CUUUGGGUGUCCGUGUUUCACUUUUUCCUUUUAUAUUUGCUUAUGGUGACAAUAUAUAGAUUAUAUA | |
| | UUGACACC | |
| 425 | ACCUUCCUCCAUGUAACUUAGAAGAUGUACACUCAGACCAAUAGGUGAUAAUCCUCCAGAUUAUCUA | rhinovirusA55 |
| | AGGUCAAGCACUUCUGUUUCCCCGGUCUAAACUGAUAUGCUCCAACAGGGCAAAAACAGUGCAGAUC | |
| | GUUAUCCGCAGGAUGCCUACGCAAAGUCUAGUAACCCCUUUGAAGAUAUUUGGUUGGUCGCUCCACU | |
| | AUUCCCCAUAGUAGACCUGGCAGAUGAGGCUAGAAAUUCCCCACUGGUGACAGUGUUCUAGCCUGCG | |
| | UGGCUGCCUGCACACCCUAUUGGGUGUGAAGCCAAAUAUUGGACAAGGUGUGAAGAGCCCCGUGUGC | |
| | UCAUCCUGAGUCCUCCGGCCCCUGAAUGUGGCUAACCUUAACCCUGCAGCUAGUGCAUGCAAUCCAG | |
| | CAUGUAGCUAGUCGUAAUGAGCAAUUGCGGGAUGGGACCAACUACUUUGGGUGUCCGUGUUUCACUU | |
| | UUUACCUUAUAUUUGCUUAUGGUGACAAUAUAUAUUGCAUAUAUAUUGGCAUC | |
| 426 | UUAAAACUGgGUUCAGGUUGUUCCCACCUGGACUUCCUAUGUGGAGUUGUACACUGUUAUCCCGGUA | rhinovirusA56 |
| | ACCUUGUACGCCAGUUUUAUACACCCUUCCCUGUAACUUAGAAGUUAUAAGCACGAGGACCAAUAGG | |
| | UAGUGACUUUCCAGGUCACUAAAGGUCAAGCACUUCUGUCUCCCCGGUCAAAGUUGAUAUGCUCUCC | |
| | CCAGGGCAAAAACAAUUGAGAUCGUUAUCCGCAAUGUGCCUACGCAAAGCCUAGUAAUAUCUUAGAU | |
| | GAUAUAUGGUUGGUCGCUCCGCCAUUUCCCAUGGUAGACCUGGCAGAUGAGGCUAGAAACUCCCCAC | |
| | UGGCGACAGUGUUCUAGCCUGCGUGGCUGCCUGCGCACUCUCUGAGUGCGAAGCCAUAUAUUUGACA | |
| | AGGUGUGAAgAGCCCCGUGUGCUCACAUUGAAUCCUCCGGCCCCUGAAUGUGGCUAACCUUAAACCU | |
| | GCAGCUAGUGCACACAAACCAGUGUGUAGCUAGUCGUAACGAGCAAUUGCGGGAUGGGACCAACUAC | |
| | UUUGGGUGUCCGUGUUUCACUUUUUUCCUUUUAUAUUUGCUUAUGGUGACAAUAUAUAUAAUACAUA | |
| | UAUUGACACC | |
| 427 | UUAAAACUGGAUCCAGGUUGUUCCCACCUGGAUCUCCUACGGGAGUUGUACUCUGUUAUUUCGGUAA | rhinovirusA57 |
| | CCUUGUACGCCAGUUUUAUCUCCCCUCCCCCAAUUGUAACUUAGAAGCAAUACACAAGGACCAAUAG | |
| | GCAGUGAUUAACCAGAUCACUUAAGGUCAAGCACUUCUGUUUCCCCGGUCUGAGUUGAUAUGCUCUA | |
| | ACAGGGCAAAAACAAGUCAGAUCGUUAUCCGCAAAGUGCCUACGCAAAGCCUAGUAUCAUCUUUGAA | |
| | AAUGUAUGGUUGGUCAUUCCGCUGAACCCCACAGUAGACCUGGCAGAUGAGGCUAGAAACCCCCCAC | |
| | UGGCGACAGUGUUCUAGCCUGCGUGGCUGCCUGCACACCCUUAUGGGUGUGAAGCCAUAUAUUUGAC | |
| | AAGGUGUGAAGAGCCCCGUGUGCUCACUUUGAGUCCUCCGGCCCCUGAAUGUGGCUAACCCUAACCC | |
| | UGCAGCUAGUGCAUACAAGCCAGUAUGUUGCUAGUCGUAACGAGCAAUUGCGGGAUGGGACCAACUA | |
| | CUUUGGGUGUCCGUGUUUCACUUUUUACCUUUAUACUGCUUAUGGUGACAAUAUAUAUAUAGCAUAU | |
| | AUAUUGACACC | |
| 428 | UUAAAACUGGGAGUAGGUUGUUCCCACCUACUCUACCCACGUGGUGUUGUACACUGUUAUCACGGUA | rhinovirusA58 |
| | ACUUUGUAUGCCAGUUUUGCCACCCUUCCCCGUAGCGUAACUUAGAAGUUUGUACACUUAGACCAAU | |
| | AGGCGAUAAUCAUCCAGAUUAUCAAAGGUCAAGCACUUCUGUUUCCCCGGUCAAUGAGGAUAUGCUU | |
| | UUCCCAAGGCAAAAACCUUAGAGAUCGUUAUCCCCACAUUGCCUACACAGAGCCUAGUAUCACCUUU | |
| | GAUGUAUUUAGACUGGUCGCUCCCUGCAAACCCAGCAGUAGACCUGGCAGAUGAGGCUGGACGUUCC | |
| | CCACUGGCGACAGUGGUCCAGCCUGCGUGGCUGCCUGCCCACCCAUUUGGGUGGGAAGCCUAACUAU | |
| | UGACAUGGUGUGAAGAGCCGCGUGUGCUCAAUGUGCCUCCUCCGGCCCCUGAAUGUGGCUAACCUUA | |
| | ACCCCGCAGCCGCUGCCCAUGAUCCAGUGGGUAUACGGUCGUAACGCGCAAGUGUGGGAUGGGACCA | |
| | ACUACUUUGGGUGUCCGUGUUUCCUGUUUUUCUUUUUUUUAUGCAUUUUAUGGUGACAAUUUAUGGU | |
| | AUAUAGAUUGUCAUC | |
| 429 | UUAAUACUGGAUCCAGGUUGUUCCCACCUGGAUCUCCCAAGCGGAGUGGUACACUAUUAUCCCGGUA | rhinovirusA59 |
| | AUUUUGUACGCCAGUUUUAUAACCCCUCCCCUUUGUAACUUAGAAGAUGUAAACACAGACCAAUAGG | |
| | UGGUAACUAACCAAGUUACUAAAGGUCAAGCACUUCUGUUUCCCCGGCUGAUAUUGAUAUGCUCCAC | |
| | CAGGGCAAAAACAAUAGAAGUCGUUAUCCGCAAAGUGCCUACGCAAAGCCUAGUAACACUUUGAAUU | |
| | GUUUAUGGUUGGUCGCUCAGCUAUUCCCCCUAGUAGACCUGGCAGAUGAGGCUAGAAAUACCCCACU | |
| | GGUAACAGUGUUCUAGCCUGCGUGGCUGCCUGCGCACCCCUCCGGGUGCGAAGCCAUACACAUGACA | |
| | AGGUGCGAAGAGCCCCGUGUGCUCACUUUGAAUCCUCCGGCUCCUGAAUGUGGCUAACCUUAAACCU | |
| | GCAGCCAGUGCACAUAAGCCAAUGUGUAUCUGGUCGUAAUGAGUAAUUGCGGGAUGGGACCAACUAC | |
| | UUUGGAUGUCCGUGUUUCUUGUUUUUCCUUAAUUUUGCUUAUGGUGACAAUAUAUAGAUAAUAUAUA | |
| | UUGGCAUC | |
| 430 | UUAAAACUGGAUCCAGGUUGUUCCCACCUGGAUCUCCUAACGGGAGUAGUACACUAUUAUUACGGUA | rhinovirusA60 |
| | AUUUUGUACGCCAGUUUUAUGAUCCCCUCCCCCUUUUGUAACUUAGAAGUUAAACAAAAUGACCAAU | |
| | AGGCGGCAAUCUCCCAGAUUGUCAAAGGUCAAGCACUUCUGUUUCCCCGGUCAAAGCUGAUAUGCUC | |
| | CAACAGGGCAAAAACAGCUGAGAUCGUUACCCGCAAAGUGCCUACGCAAAGCUUAGUAACACUUUUG | |
| | AAGUUACGUGGUUGGUCGCUCAGCCAAACCCCAUGGUAGACCUGGCAGAUGAGGCUAGAAGUACCCC | |
| | ACUGGCGACAGUGUUCUAGCCUGCGUGGCUGCCUGCACACCCUCUGGGUGUGAAGCCAUAUAAUGGA | |
| | CAAGGUGUGAAGAGCCCCGUGUGCUCACUUUGAGUCCUCCGGCCCCUGAAUGUGGCUAACCUUAACC | |
| | CUGCAGCUAGUGCAUGCAAUCCAGUAUGUAUCUAGUCGUAAUGAGCAAUUGCGGGAUGGGACCAACU | |
| | ACUUUGGGUGUCCGUGUUUCCUUUUUAUCCUUAUUGAUUGCUUAUGGUGACAAUAUAUAGUAUUAUA | |
| | UAUUGACaCC | |
| 431 | UUAAAACUGGGUCUGGGUUGCUCCCACCCAGACCACCCAUGUGGUGUUGUACACUGUUAUUCCGGUA | rhinovirusA61 |
| | ACUUUGUACGCCAGUUUUGAACUCCCCUACCCCUUUUGUAACUUAGAAGCUAAACACAUCGACCAAU | |
| | AGCAGGCAAUCACCCAGAUUGCUUAUGGUCAAGUACUUCUGUUUCCCCGGUCUCCUCUGAUAUGCUC | |
| | UACCAGGGCAAAAACAGAGUAGAUCGUUAUCCGCAAGAUGCCUACGCAACGCCUAGUAACACUUUGA | |
| | AGUGUUUUUGGUUGGUCGCUCAGUUACAACCCCAGUAAUAGACCUGGCAGAUGAGGCUAGAUAUCCC | |
| | CCACUGGCGACAGUGAUCUAGCCUGCGUGGCUGCCUGCACACCCUUUAUGGGUGUGAAGCCAAACAU | |
| | AUGACAAGGUGCGAAGAGCCACGUGUGCUCAUCUUGAGUCCUCCGGCCCCUGAAUGCGGCUAACCUC | |
| | AACCCUGCAGCCAUUGCUCACAAGCCAGUGGGUAUGUGGUCGUAAUGAGAAAUUGCGGGACGGGACC | |
| | GACUACUUUGGGUGUCCGUGUUUCACUUUUUUACCUUAUUUUGCUUAUGGUGACAAUAUAUAUACAG | |
| | AUAUAUAUUGACACC | |
| 432 | UUAAAACUGGAUCCAGGUUGUUCCCACCUGGAUCUCCUACGUGGAGUUGUACUCUAUUAUUCCGGUA | rhinovirusA62 |
| | AUCUUGUACGCCAGUUUUAUAUUCCCUACCCCUUUUGUAACUUAGAAGUUAAGCACACAGACCAAUA | |
| | GGCGGUGACCACCCAAGUCACUAACGGUCAAGCACUUcUGUUUCCCCGGUUAAGAUUGAUACGCUCC | |
| | AACAGGGCGAAAACAAUUUAAAUCGUUAACCGCAAAGUGACUACGCAAAGCUUAGUAAUACCUUGAA | |
| | GAAUUUAUGGCUGGUCGUUCCGCUAUAACCCCUAGUAGACCUGGCAGAUGAGGCUAGAAAUACCCCA | |
| | CUGGUAACAGUGUUCUAGCCUGCGUGGCUGCCUGCACACCCUUUUUGGGUGUGAAGCCAUAUAUUUG | |
| | ACAAGGUGUGAAGAGCCCCGUGUGCUCACUUUGAGUCCUCCGGCCCCUGAAUGUGGCUAACCUUAAC | |
| | CCUGCAGCUAGUGUAUGUAAUCCAACAUAUGGCUAGUCGUAAUGAGCAAUUGCGGGAUGGGACCAAC | |
| | UACUUUGGGUGUCCGUGUUUCACUUUUUCCUUUUAUUUUGCUUAUGGUGACAAUAUAUAUAAUAUAU | |
| | AUAUUGGCAUC | |
| 433 | UUAAAACUGGGUCCAGGUUGUUCCCACCUGGACCUCCUAAGUGGAGUGGUACACUAUUAUUCCGGUA | rhinovirusA63 |
| | AUUUUGUACGCCAGUUUUAUCUCCCCACCCCCCUUUUGUAAUUUAGAAGUUAUGAACACAGACCAAU | |
| | AGGUGGCAACUAGCCAAGUUGUUAAAGGUCAAGCACUUCUGUCUCCCCGGCUGACGUUGAUAUGCUC | |
| | CAACAGGGCAAAAACAAUGAAAGUCGUUAUCCGCAAAGUGCCUACGCAAAGCCUAGUAACACUUUGA | |
| | AAUGUCUAUGGUUGGUCGCUCAGCUAUUCCCCAUAGUAGACCUGGCAGAUGAGGCUAGAAAUACCCC | |
| | ACUGGUAACAGUGUUCUAGCCUGCGUGGCUGCCUGCGCACCCUCCCCGGGUGCGAAGCCAUAUAUAU | |
| | GACAAGGUGCGAAGAGCCCCGUGUGCUCACUUUGAAUCCUCCGGCUCCUGAAUGUGGCUAACCUUAA | |
| | CCCCGCAGCCAGUGCACAUAAGCCAAUGUGUGGCUGGUCGUAAUGAGCAAUUGUGGGAUGGGACCAA | |
| | CUACUUUGGAUGUCCGUGUUUCCUGUUUUUCUUUUAUUCUUGCUUAUGGUGACAAUAUAUAUAGUUU | |
| | AUAUAUAUUGGCAUC | |
| 434 | UUCAAACUGGAUCUGGGUUGUUCCCACCCAGAUCACCCAAGUGGUGUAGUACUCUGUCAUUCCGGUG | rhinovirusA64 |
| | ACUUUGUACGCCAGUUUUUACACCCCCCCCUUCUUGUAACUUAGAAGAAUUAAGUUCAGACCAAUAG | |
| | GAAAACGACAAACCAGUCGUUUAAGGUCAAGCACUUCUGUUUCCCCGGUCAAUGCUGAUAUGCUUCA | |
| | CCAAGGCAAAAACAGCAGCGAUCGUUACCCGCAAGACGCCUACGCAAAGCCUAGUAAUGCCUUUGAA | |
| | GAUAUUUGUCUGGUCACUCAGGUACAAUCCCAGUACUAGACCUGGCAGAUGAGGCUGGAAAAUCCCC | |
| | ACUGGCGACAGUGUUCCAGCCUGCGUGGCUGCCUGCACACCCAUACGGGUGUGAAGCCAAAUAUUGG | |
| | ACAAGGUGUGAAGAGCCCCGUGUGCUCGUCUUGAGUCCUCCGGCCCCUGAAUGCGGCUAACCUUAAC | |
| | CCCACAGCCAUUGCUUGCAUUCCAGCAAGUCUAUGGUCGUAAUGAGCAAUUGUGGGAUGGGACCGAC | |
| | UACUUUGGGUGUCCGUGUUUCACUUUUUACCUUUUAUUAUGCUUAUGGUGACAAUAUAUAGCGUUAU | |
| | AUAUUGGCAUC | |
| 435 | UUAAAACUGGGUGUGGGUUGCACCCACCCACACUGUCUACGGACGUAGUGCUCUCAUAUUACGGUAU | rhinovirusA65 |
| | GCUUGCACGCCAGUUUGUUUCCCCUUUCCCCUUUUGUAACUAUAGAAGAUUGCACUUAAUCUCGACC | |
| | AAUAGGCAGUAGUGAUCCACACUACUUACGGUCAAGCACUUCUGUUACCCCGGUAUGCGGAGAAUAG | |
| | GCUCUGAAAAGGGCUGAAGCUUCAAUUAUCGUCAUCCGCGCAAUUACUACGCAAAGCUUAGUAACUU | |
| | CCCAAGAAAUUCUUUGGUUGGCCGUUCCGCUGGUACCCCACCAGUAGGCUUGGCAGAUGAGGCUGAG | |
| | UAAUCCCCACUGGCGACAGUGACUCAGCCUGCGUGGCUGCCUACACACCUGGUAACAGGUGUGAAGC | |
| | CAUAGAAAGGACAGGGUGUGAAGAGCCCCGUGUGCUAAUUGUGAUUCCUCCGGCCCCUGAAUGCGGC | |
| | UAAUCUAACCCCACAUCCAAUGCGCAUAAGCCAAUGCGUAUUUGGUCGUAAUGGGCAACUGUGGGAC | |
| | GGAACCGACUACUUUGGGUGUCCGUGUUUCUUUUUAUUCCUUAUAAUUAGUUCAUGGUGACAAGUAU | |
| | AAGUGUUUAUAUUUGUCAUC | |
| 436 | UUAAAACUGGAUCUAGGUUGUUCCCACCUAGAUCUCCCAUGUGGAGUAGUACUCUAUUAUUACGGUA | rhinovirusA66 |
| | AUUUUGUACGCCAGUUUUAUAUCCCCUUCCCCUUUUGUAACUUAGAAGUCGUGAACACAGACCAAUA | |
| | GGUGGCGAUCACCCAGAUCGCUUAAGGUCAAGCACUUCUGUUUCCCCGGUCAACGUUGAUAUGCUCC | |
| | ACCAGGGCAAAAACAACGGAGAUCGUUAUCCGCAAGGUGCCUACGCAAAGCCUAGUAACCUCUUGGA | |
| | GGAUAUAUGGUUGGUCGCUCAGCUAUACCCCAUAGUAGACCUGGCAGAUGAGGCUAGAAACUCCCCA | |
| | CUGGCGACAGUGUUCUAGCCUGCGUGGCUGCCUGCGCACUCUUAUGAGUGCGAAGCCAUAUAUUUGA | |
| | CAAGGUGUGAAGAGCCCCGUGUGCUCACCUUGAGUCCUCCGGCCCCUGAAUGUGGCUAACCCUAACC | |
| | CUGCAGCUAGUGCAUGAAAUCCAUCAUGUUGCUAGUCGUAAUGAGCAAUUGCGGGAUGGGACCAACU | |
| | ACUUUGGGUGUCCGUGUUUCACUUUUUCCUUUUAUAUUUGCUUAUGGUGACAAUAUAUAUAUAACAU | |
| | AUAUAUUGGCAUC | |
| 437 | UUAAAACUGGAUCUAGGUUGUUCCCACCUAGAUCUCCCAAGUGGAGUGGUACACUAUUAUCACGGUA | rhinovirusA67 |
| | AUUUUGUACGCCGGUUUUAUCUCCCCUUACCCCAAGUAACUUAGAAGUAUUACAUAAAGACCAACAG | |
| | GCAGUGAUUAUCCAGAUCACUAAAGGUCAAGCACUUCUGUCUCCCCGGUCAAAGUUGAUAUGCUCCA | |
| | ACAGGGCAAAAACAACUGAGAUCGUUAUCCGCAAAGUGCCUACGCAGAGCCUAGUAACUACCUUGAA | |
| | AUUACUUGGUUGGUCGUUCCACCAAACCCCAUGGUAGACCUGGCAGAUGAGGCUAGAAAUUCCCCAC | |
| | UGGUAACAGUGUUCUAGCCUGCGUGGCUGCCUGCACACCCUUUGGGUGUGAAGCCAAGUAAUGGACA | |
| | GGGUGUGAAGAGCCGAGUGUGCUCACUUUGAGUCCUCCGGCCCCUGAAUGUGGCUAACCUUAACCCU | |
| | GCAGCUAGAGCGCGCAAACCAGCGUGUAUCUAGUCGUAAUGAGCAAUUGCGGGACGGGACCAACUAC | |
| | UUUGGGUGUCCGUGUUUCUUUUUAUUCUUAUAUUUGCUUAUGGUGACAAUAUAUAAUUAUUUGUAUA | |
| | UUGGCACC | |
| 438 | UUAAAACUGGGUCCAGGUUGUUCCCACCUGGAUCUCCUACGUGGAGUUGUACUCUAUUAUUCCGGUA | rhinovirusA68 |
| | AUUUUGUACGCCAGUUUUAUCUCCCCUACCCCUCUAUGUAACUUAGAAGUUUAUGCACAGCGGCCAA | |
| | UAGGUGACAUUUAUCCAAAAUGUCAAAGGUCAAGCACUUCUGUUACCCCGGUUAGAAUUGAUAUGCU | |
| | CCAACAGGGCAAAAACAAUUUUGAUCGUUAUCCGCAAGGCAACUACGCAAAGCCUAGUACCAUUUUU | |
| | GAUGAUGUGUGGUUGGUCGCUCCACUGUGAACCCGACAGUAGACCUGGCAGAUGAGGCUAGAAAUUC | |
| | CCCACUGGUAACAGUGUUCUAGCCUGCGUGGCUGCCUGCGUACCCUUUUGCGGGUACGAAGCCAUAC | |
| | AUUUGACAAGGUGUGAAGAGCCCCGUGUGCUUGCCUUGAGUCCUCCGGCCCCUGAAUGUGGCUAACC | |
| | UUAACCCUGCAGCUAGUGUGCACAAUCCAGUGCAUGGCUAGUCGUAACGAGCAAUUGCGGGAUGGGA | |
| | CCAACUACUUUGGGUGUCCGUGUUUCUUGUUUUUCUUUUAUAUUUGCUUAUGGUGACAAUAUAUACA | |
| | UAAUAUAUAUAUUGGCACC | |
| 439 | UUAAAACUGAGUGUGGGUUGUUCCCACCCACACUGUCUACGGACGUAGUGCUCUCAUAUUCCGGUAU | rhinovirusA71 |
| | GUUUGCACGCCAGUUUGCCUCUCCUUUUCCCACGUAACGUUAGAAGCUUAUGCACUAAAUCGACCAC | |
| | UAGGUGAGUGAUAAACCAUAUCAUUCUUUGGUCAAGCACUUCUGUUUCCCCGGUAUGCAGGGAAUAG | |
| | GCCCUCCCCAGGGCUGAAGCCUUAAUUAUCGUUAUCCGCACAUUUACUACGCAAAGCCUAUUAGCGU | |
| | UCUGGAAGUUCACUGGCUGGUCGUUCCGCUAGACUAACCCGUCUAGUAGACCUGGCAGAUGAGGCUG | |
| | GAUAAACCCCACUGGCGACAGUGAUCCAGCCUGCGUGGCUGCCUGCACACCUGGAAACAGGUGUGAA | |
| | GCCCAUGAAUGGACAGGGUGUGAAGAGCCCCGUGUGCUAAGUGUGAGUCCUCCGGCCCCUGAAUGCG | |
| | GCUAAUCUAACCCCACAGCUAAUACACACAAACCAGUGUGGAUUUAGUCGUAAUGGGUAACUGUGGG | |
| | ACGGAACCGACUACUUUGGGUGUCCGUGUUUCCUUUUAUUCCUUUUAUAUUGCUUAUGGUGACAGGU | |
| | AUAUAGAUAUAUAUUUGUUACC | |
| 440 | UUAAAACUGGGUUUGGGUUGAUCCCACCCAAACCUCCCAAGCGGAGUGGUACUCUGUUAUUUCGGUA | rhinovirusA74 |
| | ACUUUGUACGCCAGUUUUAUAUUCCCUCCCCCCUUUAAACUGUAAAGCUAAGCACACAGACCAAUAG | |
| | CUGACAAUCAUCCAGAUUGUUAAUGGUCAAGCACUUCUGUUUCCCCGGUCAACGUUGAUAUGCUCCA | |
| | ACAGGGCAAAAACAACUGCGAUCGUUAUCCGCAAAGUGCCUACGCAAAGCCUAGUAGCACCUUUGAA | |
| | GUCAUUUGGUUGGUCGCUCCGCUGUUCCCCACAGUAGACCUGGCAGAUGAGGCUAGAAAUACCCCAC | |
| | UGGUGACAGUGUUCUAGCCUGCGUGGCUGCCUGCACACCCUUUGGGUGUGAAGCCAAAUCAUGGACA | |
| | AGGUGUGAAGAGCCCCGUGUGCUCACUUUGAGUCCUCCGGCCCCUGAAUGUGGCUAACCUUAACCCU | |
| | GCAGCUAGAGUGCACAAUCCAGUGCAUUUCUAGUCGUAAUGAGCAAUUGCGGGAUGGGACCAACUAC | |
| | UUUGGGUGUCCGUGUUUCACUUUUUACCUAUUAUUUGCUUAUGGUGACAAUAUAUAUAAUUAUAUAU | |
| | UGGCACC | |
| 441 | UUAAAACUGGAUCCAGGUUGUUCCCACCUGGAUCACCCAAUGGGUGUAGUAUACUGUUAUUACGGUA | rhinovirusA75 |
| | ACUUUAUACGACGGUUUUAUCUCCCCCCCCCCCUUAUGUAACUUAGAAGUUUUGCACACCGACCAAU | |
| | AGUGGACAAUCAGCCAGAUUGUUUUAGGUCAAGCACUACUGUUUCCCCCGGUCAACGUUGAUAUGCU | |
| | CCGACAGGGCAAAAACAACAGCGAUCGUUAUCCGCAAGGUGCCUACACAGAGCUUAGUACCGCCUUA | |
| | GAAGGAUUGUGGUUGGUCGCUCAGUUGCAACCCCAGCAAUAGACCUGGCAGAUGAGGCUGGACAUUC | |
| | CCCACUGGUGACAGUGGUCCAGCCUGCGUGGCUGCCUGCACACCUCUAUGAGGUGUGAAGCCAGAAU | |
| | UUCGACAAGGUGCGAAGAGCCGCGUGUGCUCACCUUGAAUCCUCCGGCCCCUGAAUGCGGCUAACCU | |
| | UAAACCCGCAGCCAUGGUCCACAAUCCAGUGGAUGUAUGGUCGUAAUGAGUAAUUGUGGGAUGGGAC | |
| | CGACUACUUUGGGUGUCCGUGUUUCCUGUUUUUUCUUUUAUACUGCUUAUGGUGACAAUAUAUAUGA | |
| | UAUAUAUUGGCACC | |
| 442 | UUAAAACUGGAUCCAGGUUGUUCCCACCUGGAUCUCCUACAAGGAGUUGUACUCUAUUAUUGCGGUA | rhinovirusA76 |
| | AUUUUGUACGCCAGUUUUAUCUCCCCUCCCCGAUAUGUAACUUAGAAGCUGAACACAAAGACCAAUA | |
| | GGUGACAUUUAAUCCAAAAUGUUAAAGGUCAAGCACUUCUGUUUCCCCGGCCAAAGUUGAUAUGCUC | |
| | CAACAGGGCAAAAACAGCUGCGGUCGUUAUCCGCAAAACAACUACGCAAAGCCUAGUACCACCUUCU | |
| | AGAGCAUGUGGCUGGUCGCUCCACUAUAAACCCAAUAGUAGACCUGGCAGAUGAGGCUAGGAGCUCC | |
| | UCACUGGUAACAGUGUCCUAGCCUGCGUGGCUGCCUGCGCACCCUGACAGGUGCGAAGCCAAAUGUU | |
| | UGACAAGGUGUGAAGAGCCCCGUGUGCUUGUUUUGAGUCCUCCGGCCCCUGAAUGUGGCUAACCUUA | |
| | ACCCUGCAGCUAGUGUGUACAAUCCAGUGCAUAGCUAGUCGUAAUGAGCAAUUGCGGGAUGGGACCA | |
| | ACUACUUUGGGUGUCCGUGUUUCUUAUUUUUUCCUUAUAUUUGCUUAUGGUGACAAUGUAUAUAUAA | |
| | CGUAUAUAUUGGCAUC | |
| 443 | UUAAAACUGGAUCCAGGUUGUUCCCACUUGGAUCCCCCAAGAGGGGUAGUACUCUAUUAUUACGGUA | rhinovirusA77 |
| | AUUUUGUACGCCAGUUUUAUCUCCCUUACCCCCUUUGUAACUUAGAAGAUUUAAACACAGACCAAUA | |
| | GGCGACGAUCAUCCAGAUCGUUUAAGGUCAAGCACUUCUGUUUCCCCGGUCAAUGCUGAUAUGCUCU | |
| | ACCAGGGCAAAAACAGUAGAGAUCGUUAUCCGCAAGGUGCCUACACAAAGCUUAGUAACAUUUUGAA | |
| | GAGCAUUUGGUUGGUCGCUCAGCUAUUACCCCAUAGUAGACCUGGCAGAUGAGGCUAGAAGUUCCCC | |
| | ACUGGCGACAGUGUUCUUGCCUGCGUGGCUGCCUGCGCACUCUCAUGAGUGCGAAGCCAUAUGUUUG | |
| | ACAAGGUGUGAAGAGCCCCGUGUGCUCACCUUGAGUCCUCCGGCCCCUGAAUGUGGCUAACCUUAAC | |
| | CCUGCAGCUAGUGCAUGAAAUCCAUCAUGUAACUAGUCGUAAUGAGCAAUUGCGGGAUGGGACCAAC | |
| | UACUUUGGGUGUCCGUGUUUCACUUUUUCCUUUUAUAUUUGCUUAUGGUGACAAUAUAUAUAGCAUA | |
| | UAUUGACACC | |
| 444 | UUCAAACUGAAUCAUGGUUGUUCCCACCAUGAUUACCUACGGGUGUAGUACACUUGUAUCCCGGUAC | rhinovirusA78 |
| | AUUUGUGCGCCAGUUUUAUAUCCCCUACCCCAAUUGUAACUUAGAAGUUAAGCAACACAAGAUCAAU | |
| | AGGCGGUGGUUAUCCAAACCAUCAAUGAUCAAGUACUUCUGUCUCCCCGGUAGGUGUGAAUAUGCUU | |
| | UAACCAAGGCAGAAGCUAAUCCUAUCGUUAUCCGCAAGGUGCCUACGGGAAGCCUAUUAACACUUUG | |
| | AAAGAAAUUUGGCUGGUCGCUCAGUUGCGCAAUCCCACGCAAUAGACCUGGCAGAUGGGGCUAGACA | |
| | AACCCCACCAGCGAUGGUGGUCUAGCCCGCGUGGCUGCCUGCACACCCUUUUUUGGGUGUGAAGCCA | |
| | GAAAUUGGACAGGGUGCGAAGAGCCUACUGUGCUCACCUUGAGUCCUCCGGCCCCUGAAUGUGGCUA | |
| | AUCCUAACCCUGCAGCUGUUGCACAAGAUCCAUUGUGUAUACAGUCGUAAUGAGCAAUUGCGGGAUG | |
| | GAACCAACUACUUUGGGUGUCCGUGUUUCCUUGUUUUCUUUUAUCAUUGCUUAUGGUGACAAGUAUA | |
| | UAUAGAUAUAUUUGUCAUC | |
| 445 | UUAAAACUgAAUCCAGGUUGUUCCCACCUGGAUUUCCCACGAGGAGCAGUACUCUACUAUUCCGGUA | rhinovirusA80 |
| | GUUUUGUACACCAGUUUUAUCUCCCUUCCCCGUGUAACUUAGAAGACAAGCACUUCAGACCAAUAGG | |
| | AUGGUAACUAACCAAGUUAUCAAAGGUCAAGCACUUCUGUUUCCCCGGUCAAAAUUGAUAUGCUCCA | |
| | CCAGGGCAAAAACAGUUUUGAUCGUUAUCCGCAAAGUGCCUACGCAAAGCUUAGUAACACUUUGAAG | |
| | AAUGUGUGGUUGGUCGUUCCGCUAUUCCCCAUAGUAGACCUGGCAGAUGAGGCUAGAAAAUCCCCAC | |
| | UGGUGACAGUGUUCUAGCCUGCGUGGCUGCCUGCGCACCUCUUUGAGGUGCGAAGCCAUUCCUUUGA | |
| | CAAGGUGUGAAGAGCCCCGUGUGCUCACUUUGAGUCCUCCGGCCCCUGAAUGUGGCUAACCUUAACC | |
| | CUGCAGCUAGUGCAUGCAAUCCAGCAUGUCUCUAGUCGUAAUGAGUAAUUGCGGGAUGGGACCAACU | |
| | ACUUUGGGUGUCCGUGUUUCUCUUUUUACUUUUAAAUUGCUUAUGGUGACAAUAUAUAUAGUACAUA | |
| | UAUAUUGACACC | |
| 446 | UUAAAACUGGAUCUaGGUUGUUCCCACCCAGAUCACCUACAUGGUGUUGUACACUAUUAUUACGGUA | rhinovirusA81 |
| | AUUUUGUACGCCAGUUUUAUACCCCCUUCCCAAAAUGUAACUUAGAAGCAAUACAAACUGAUCAAUA | |
| | GGAAGCAAUCAUCCAGAUUGCUAACGAUCAAGCACUUCUGUCUCCCCGGUCAAGGUUGAUAUGCUCC | |
| | ACCAGGGCAAAAACAGCUAAGAUCGUUAUCCGCAAGAUGCCUACGCAAAGCCUAGUAAUGCUUUCGA | |
| | AGAUACUUGGUUGGUCGCUCAGCUGUGCACCCGACAGUAGACCUGGCAGAUGAGGCUAGAAGUACCC | |
| | CUCUGGUGACAGAGUUCUAGCCUGCGUGGCUGCCUGCACACCCUAUUGGGUGUGAAGCCAAAUAUUG | |
| | GACAAGGUGUGAAGAGCCCCGUGUGCUCAUCUUGAGUCCUCCGGCCCCUGAAUGUGGCUAACCUUAA | |
| | ACCUGCAGCCAGUGCACACAAACCAGUGUGUAGCUGGUCGUAACGAGCAAUUGCAGGAUGGGACCAA | |
| | CUACUUUGGGUGUCCGUGUUUCACUUUUUACCCUUAUAUUGCUUAUGGUGACAAUAUAUAUAGUAUA | |
| | UAUAUAUUGUUAUC | |
| 447 | UUAAAACUGGGAGUGAGUUGUCCCCACUCACUCUACCCACGCGGUGUUGUACACUGUUAUUACGGUA | rhinovirusA82 |
| | ACUUUGUAUGCCAGUUUUUGUUCCCCUAUCCCCCUUUAAAGUAACUUAGAAGCAUGAACUUACAGAC | |
| | CAAAAGGUGAUGAUCACCCAGAUCAUUAAAGGUCAAGCACUUCUGUUUCCCCGGUCAAUGUUGAUAU | |
| | GCUUCAACAAGGCAAAAACAACAGCGAUCGUUAUCCGCAAGAUGCCUACACAAAGCUUAGUACCUCC | |
| | UUUGAUGGUAUUUGUCUGGUCGCUCAGGUGUUUCCCCAACACUAGACCUGGCAGAUGAGGCUAGAAA | |
| | CUCCCCACUGGCGACAGUGUUCUAGCCUGCGUGGCUGCCUGCACACCCUUUCGGGUGUGAAGCCAAA | |
| | UACUGGACAAGGUGCGAAGAGCCCCGUGUGCUCAUCUUGAGUCCUCCGGCCCCUGAAUGCGGCUAAC | |
| | CUUAAACCCGCAGCCAUUACUCACAAUCCAGUGAGCAGAUGGUCGUAAUGAGCAAUUGUGGGAUGGG | |
| | ACCGACUACUUUGGGUGUCCGUGUUUCAUGUUUUACUUUAUGACUUGCUUAUGGUGACAAUAUAUAG | |
| | AGAUAUAUAUUGGCAUC | |
| 448 | UUAAAACUGGAUCCAGGUUGUUCCCACCUGGAUUUCCUACAUGGAGUUGUACUCUGUUAUUCCGGUA | rhinovirusA85 |
| | ACCUUGUACGCCAGUUUUAUAUCCCUUCCCCCUUGCAACUUAGAAGUUAAGCACGCAGACCAAUAGG | |
| | UGGUGAUUAUCCAAAUCACUAACGGUCAAGCACUUCUGUUUCCCCGGUUUAAACUGAUACGCUCCAA | |
| | CAGGGCGAAAACAGUGCAAAUCGUUAUCCGCAAGGUGCCUACACAAAGCUUAGUAGCGCUUUGGAGA | |
| | AUGUGUGGUUGGUCGCUCAGCUAUACCCCCUAGUAGACCUGGCAGAUGAGGCUAGAAGCACCCCACU | |
| | GGCGACAGUGUUCUAGCCUGCGUGGCUGCCUGCACACCCUUAUGGGUGUGAAGCCAUACAUUUGACA | |
| | AGGUGUGAAGAGCCCCGUGUGCUCACUUUGAGUCCUCCGGCCCCUGAAUGUGGCUAACCUUAACCCU | |
| | GCAGCUGGUGCAUGUAAUCCAACAUGUUGCUAGUCGUAAUGAGCAAUUGCGGGAUGGGACCAACUAC | |
| | UUUGGGUGUCCGUGUUUCACUUUUUACCUUUUAAUUUGCUUAUGGUGACAAUAUAUAUACAACAUAU | |
| | AUAUUGGCAUC | |
| 449 | UUAAAACUGGGAGUGAGUUGUCCCCACUCACUCUACCCACGCGGUGUUGUACACUGUUAUUACGGUA | rhinovirusA88 |
| | ACUUUGUAUGCCAGUUUUUGUUCCCCUAUCCCCCUUUAAAGUAACUUAGAAGCAUGAACUUACAGAC | |
| | CAAAAGGUGAUGAUCACCCAGAUCAUUAAAGGUCAAGCACUUCUGUUUCCCCGGUCAAUGAGGAUAU | |
| | GCUUUACCCAAGGCAAAAACCUUAAAGAUCGUUAUCCGCACAUUAACUACGCAAAGCCUAGUAUCGC | |
| | CUUUGAUGUAUUAAGACUGGUCGUUCCCUGCAUACCCAGCAGUAGACCUGGCAGAUGAAGCUGGACG | |
| | UUCCCCACUGGCGACAGUGGUCCAGCCUGCGUGGCUGCCUGCUCACCCAUUUGGGUGAGAAGCCUUA | |
| | UUAUUGACAAGGUGUGAAGAGCCCCGUGUGCUUAAUGUGAAUCCUCCGGCCCCUGAAUGUGGCUAAC | |
| | CUUAACCCCGCAGCCGUUACUCACAAUCCAGUGAAAUGACGGUCGUAACGCGAAAGUGUGGGAUGGG | |
| | ACCAACUACUUUGGGUGUCCGUGUUUCCUAUUUUUUCCUUAAUGUGUGUUAUAUGGUGACAAUUUAU | |
| | AGCGUAUAAGUUGUCAUC | |
| 450 | UUAAAACUGGGAGUGGGUUGUUCCCACUCACUCCACCCAUGCGGUGUUGUACUCUGUUAUUACGGUA | rhinovirusA89 |
| | ACUUUGUACGCCAGUUUUUCCCACCCUUCCCCAUAAUGUAACUUAGAAGUUUGUACAAUAUGACCAA | |
| | UAGGUGACAAUCAUCCAGACUGUCAAAGGUCAAGCACUUCUGUUUCCCCGGUCAAUGAGGAUAUGCU | |
| | UUACCCAAGGCAAAAACCUUAGAGAUCGUUAUCCCCACACUGCCUACACAGAGCCCAGUACCAUUUU | |
| | UGAUAUAAUUGGGUUGGUCGCUCCCUGCAAACCCAGCAGUAGACCUGGCAGAUGAGGCUGGACAUUC | |
| | CCCACUGGCGACAGUGGUCCAGCCUGCGUGGCUGCCUGCUCACCCUUCUUGGGUGAGAAGCCUAAUU | |
| | AUUGACAAGGUGUGAAGAGCCGCGUGUGCUCAGUGUGCUUCCUCCGGCCCCUGAAUGUGGCUAACCU | |
| | UAACCCUGCAGCCGUUGCCCAUAAUCCAAUGGGUUUGCGGUCGUAAUGCGUAAGUGCGGGAUGGGAC | |
| | CAACUACUUUGGGUGUCCGUGUUUCCUGUUUUUCUUUUGAUUGCAUUUUAUGGUGACAAUUUAUAGU | |
| | GUAUAGAUUGUCAUC | |
| 451 | UUAAAACUGGGUUCAGGUUGUUCCCACCUGAAUCUCCUACAUGGAGUUGUACUCUAUUAUCCCGGUA | rhinovirusA90 |
| | AUUUUGUACGCCAGUUUUAUCCUCCCCUCCCCAUGUAACUUAGAAGCUGUACACAACGACCAACAGG | |
| | UAACAUUCAUCCAGAGUGUUAAAGGUCAAGCACUUCUGUUUCCCCGGUCAAAGUUGAUAUGCUCCAA | |
| | CAGGGCAAAAACAACUGCGAUCGUUAUCCGCAGAGCAACUACGCAAAGCUUAGUACCACUUUCAAUG | |
| | GUGUAUGGCUGGUCGCUCCACUAUAAACCCAAUAGUAGACCUGGCAGAUGAGGCUAGAAACUCCCCA | |
| | CUGGUAACAGUGUUCUAGCCUGCGUGGCUGCCUGCGUGCCCUUUUGGGCACGAAGCCUUACAUUUGA | |
| | CAAGGUGCGAAGAGCCCCGUGUGCUUGCUUUGAGUCCUCCGGCCCCUGAAUGUGGCUAACCUUAACC | |
| | CUGCAGCUAGUGUGUGCAAUCCAGCACAUGGCUAGUCGUAAUGAGCAAUUGCGGGAUGGGACCAACU | |
| | ACUUUGGGUGUCCGUGUUUCUUGUUUUUCUUUUAUAUUUGCUUAUGGUGACAAUAUAUAUAGAAUAU | |
| | AUAUAUUGGCAUC | |
| 452 | UUCAAACUGGAUCUGGGUUGUUCCCACCCAGAUCACCCAAGAGGUGUAGUGCUCUGUCAUUCCGGUG | rhinovirusA94 |
| | ACCUUGCACGCCAGUUUUAUCACCCCUUCCCUCUUGUAACUUAGAAGAAUUAAACUCAGACCAAUAG | |
| | GAAGCGACAAGCCAGUCGUGACAGGUCAAGCACUUCUGUUUCCCCGGUCAAAGUUGAUAUGCUUCAC | |
| | CAAGGCAAAAACAACAGCGAUCGUUAUCCGCAAGAUGCCUACGCAAAGCCUAGUAGUGCCUUUGAAA | |
| | AUAUUUGUCUGGUUGCUCAAGUACAACCCCAGUACUAAACCUGGCAGAUGAGGCUGGAGCUUCCCCA | |
| | CUGGUAACAGUGUUCCAGCCUGCGUGGCUGCCUGCACACCCUUACGGGUGUGAAGCCAAAUAUUGGA | |
| | CAAGGUGUGAAGAGCCCCGUGUGCUCAUCUUGAGUCCUCCGGCCCCUGAAUGCGGCUAACCUUAACC | |
| | CCACAGCCAUUGCUCACAAUCCAGUGAGUGUAUGGUCGUAACGAGCAAUUGUGGGAUGGGACCGACU | |
| | ACUUUGGGUGUCCGUGUUUCUCUUUUUUCCUUUUUAAUUCUGCUUAUGGUGACAAUAUAUAGUGUUA | |
| | UAUAUUGGCAUC | |
| 453 | UUAAAACUGGGUCUGGGUUGUCCCCACCUAGAUCACCCAAGUGGUGUAGUACACUGUUAUUUCGGUA | rhinovirusA96 |
| | ACCUUGUACGCCAGUUUUAUAACCCCCUCCCCAAAUGUAACUUAGAAGCUGUACACAACGACCAAUA | |
| | GAGGGUAAUCAUCCAGAUUACUUACGGUCAAGCACUUCUGUCUCCCCGGUUUACUUUGAUAUGCUCC | |
| | AACAGGGCAAAAACAAAGUGAAUCGUUACCCGCAAGAUGCCUACGCAACGCCUAGUAACAUCCUGAA | |
| | UUGUUUAUGGUUGGUCGCUCAAUUACAACCCCAGUAAUAGACCUGGCAGAUGAGGCUAGAUAUUCCC | |
| | CACUGGCGACAGUGAUCUAGCCUGCGUGGCUGCCUGCACACCCCUUGUGGGUGUGAAGCCAUAAUCA | |
| | UGACAGGGUGCGAAGAGCCACGUGUGCUCAUCUUGAGUCCUCCGGCCCCUGAAUGCGGCUAACCCUA | |
| | ACCCUGCAGCCAUUGCCUACAAACCAGUGGGUAUGUGGUCGUAACGAGAAAUUGCGGGAUGGGACCG | |
| | ACUACUUUGGGUGUCCGUGUUUCACUUUUUACCUUAUAUUUGCUUAUGGUGACAAUAUAUACGAGUU | |
| | UAUAUAUUGACACC | |
| 454 | UUAAAACUGAAUCCAGAUUGUUCCCAUCUGGAUUUCCUACAUGGAGUUGUACUCUAUUAUUCCGGUA | rhinovirusA100 |
| | AUUUUGUACGCCAGUUUUAUCACCCCUUCCCCCGUAACUUAGAAGUUUGAAACAAAAGACCAAUAGG | |
| | AGGUAACUAUCCAAGUUACUAUAGGUCAAGCACUUCUGUUUCCCCGGUCAAAGUUGAUAUGCUCCAC | |
| | CAGGGCAAAAACAAUUGAGAUCGUUAUCCGCAAAGUGCCUACGCAAAGCCUAGUAGUAUCUUGAAAA | |
| | GCAUGUGGUUGGUUGCUCCGCUGUACCCCACAGUAAACCUGGCAGAUGAGGCUAGAAGUUCCCCACU | |
| | GGUGACAGUGUUCUAGCCUGCGUGGCUGCCUGCGCACUCUUUGAGUGCGAAGCCAUAUGUUUGACAA | |
| | GGUGUGAAGAGCCCCGUGUGCUCACUUUGAGUCCUCCGGCCCCUGAAUGUGGCUAACCUUAACCCUG | |
| | CAGCUAGUGCAUGCAAUCCAGCAUGUGGCUAGUCGUAAUGAGCAAUUGCGGGAUGGGACCAACUACU | |
| | UUGGGUGUCCGUGUUUCACUUUUUUCCUUUUAUAAUUGCUUAUGGUGACAAUAUAUAGUGAUAUAUA | |
| | UUGACACC | |
| 455 | UUAAAACUGGAUCAUGGUUGUUCCCACCAUGAUUACCUACGGGUGUAGUAUACUUGUAUCCCGGUAC | rhinovirusA101 |
| | AUUUAUACGCCAGUUUUAUAACCCCCACCCCAAAUGUAACUUAGAAGCAUGUACACAAUGAUCAAUA | |
| | GGGUGGUGGCUAUCCAAGUCAUCUAAGAUCAAGCACUUCUGUCUCCCCGGUGAGUGUGAAUAUGCUU | |
| | UACCCAAGGCAAAAGCUAAUCUCAUCGUUAUCCGCAAGGUGCCUACGGGAAGCCUAGUAACAUUCUG | |
| | AAGAUAUUUUGGUUGGUCGAUCAGUUGCACUCACCCAUGCAAUAGACCUGGCAGAUGGGGCUAGGCA | |
| | UACCUCACUGGUAACAGUGGCCUAGCCCGCGUGGCUGCCUGCACACCUUCGGGUGUGAAGCCAUAAU | |
| | AUUGACAGGGUGCGAAGAGCCUACUGUGCUCACCUUGAGUCCUCCGGCCCCUGAAUGUGGCUAAUCC | |
| | UAACCCCGUAUCCAUUGCACACAAACCAGUGUGUCUAUGGUCGUAAUGGGUAACUACGGGAUGGAAC | |
| | CAACUACUUUGGGUGUCCGUGUUUCUUGUUUUUCUUUAAUACUGCUUAUGGUGACAUUAUAUAUUGA | |
| | UAUAGUGUCAUC | |
| 456 | UGUACCCACCCACACUGUCCACGGACGUAGUGCUCUCAUAUUACGGUAUGUUUGCACGCCAGUUUUU | rhinovirusA102 |
| | CCCCCCCUUCCCCCUUUUGUAACUUUAGAAGCUUGUACUUAAUCUGACCAAUAGGCAGUAGUAAUCC | |
| | AUACUACUUACGGUCAAGCACUUCUGUUACCCCGGUAUGCGGAGAAUAGGCUCUAAAAGGGCUGAAG | |
| | CUUCAAGUAUCGUUAUCCGCACAAUUACUACGCAAAGCUUAGUAACUUCCCAAGAAAUUCUUUGGUU | |
| | GGUCGCUCCGCUGGUACCCCACCAGUAGACUUGGCAGAUGAGGCUGAGUAAUCCCCACUGGCGACAG | |
| | UGACUCAGCCUGCGUGGCUGCCUACACACCUGGCAACAGGUGUGAAGCCAUAGAAAGGACAGGGUGU | |
| | GAAGAGCCCCGUGUGCUAAUUGUGAUUCCUCCGGCCCCUGAAUGCGGCUAAUCCAACCCCACAUCCA | |
| | AUGCGCAUAAACCAAUGCGUAUUUGGUCGUAAUGGGUAACUGUGGGACGGAACCGACUACUUUGGGU | |
| | GUCCGUGUUUCCUUUUAUUCUUUAAAUUAGUUCAUGGUGACAAGUAUAAGAAUUUAUAUUUGUCAUC | |
| 457 | UUAAAACUGAGUGUGGGUUGUCCCCACCCACACUGUCUACGGACGUAGUACUCUCAUAUCACGGUAU | rhinovirusA103 |
| | GUUUGUACGCCAGUUUGCCUCCCCUCCCAAGUGUAACUUAGAAGUUUGUACUUUAUAAAGGCCAAUA | |
| | GGCGGGUAGUCAACCACACUACUCAUGGUCAAGCACUUCUGUUUCCCCGGUAUGCGAAGAAUAAGCU | |
| | UUAACAAAGGCUGAAGCUUCAAUUAUCGUUAUCCGCAUGAUAACUACGCAAAGCUUAGUAGCAUUUU | |
| | GAAAGUUUAUUGGUUGGUCGCUCCACUGGAAAACCCCCCCAGUAGACCUGGCAGAUGAGGCUAAGUA | |
| | AUCCCCACUGGCGACAGUGACUUAGCCUGCGUGGCUGCCUGCACACCUGGAGACAGGUGUGAAGCCA | |
| | AGUAAUGGACAAGGUGUGAAGAGCCCCGUGUGCUUAUCAUGAGUCCUCCGGCCCCUGAAUGCGGCUA | |
| | AUCCAACCCCACAGCUAUUGCACAUAAUCCAAUGUGUAUUUAGUCGUAACGGGCAACUGUGGGACGG | |
| | AACCGACUACUUUGGGUGUCCGUGUUUCCUUUAUUCUCAUUUAAGUUUCCUAUGGUGACAAAUAUAA | |
| | GUGAUUAUAUCUGUCAUC | |
| 458 | AUCCGCAAAGCAACUACGCAAAGCCUAGUAUCACUUUUGAGAAUAUAUGGUUGGUCGCUCCACUAUA | rhinovirusA104 |
| | AACCCAAUAGUAGACCUGGCAGAUGAGGCUAGAAGUUCCCCACUGGUAACAGUGUUCUAGCCUGCGU | |
| | GGCUGCCUGCGUGCUCCUUGUGGGCACGAAGCCAUAUAUUUGACAAGGUGUGAAGAGCCCCGUGUGC | |
| | UUGUUUUGAUUCCUCCGGCCCCUGAAUGUGGCUAACCUUAACCCUGCAGCUAGCGUGUGCAAUCCAG | |
| | CACAAAGCUAGUCGUAAUGAGCAAUUGCGGGAUGGGACCAACUACUUUGGGUGUCCGUGUUUCUUGU | |
| | UUUUCUUUUAUACUUGCUUAUGGUGACAAUAUAUAUAAUAUAUAUAUUGGCAUC | |
| 459 | UUGUUUGAUGCCAGUUUUAUCUCCCCCCCCAAUUGCAACUUAGAAGAUGUACACAAAGACCAAUAGG | rhinovirusA105 |
| | CAGUGGUCACCCAGACCACUGAAGGUCAAGCACUUCUGUCUCCCCGGUCAAAGUUGAUAUGCUCCAA | |
| | CAGGGCAAAAACAACUUAGAUCGUUAUCCGCAAAGUGCCUACGCAAAGCUUAGUACCAUCUUUGAGA | |
| | GCCUAUGGUUGGUCGCUCCACUGAACCCCACAGUAGACCUGGCAGAUGAGGCUAGAAGACCCCCACU | |
| | GGCGACAGUGUUCUAGCCUGCGUGGCUGCCUGCACACCCUUACGGGUGUGAAGCCAUAUGUUUGACA | |
| | AGGUGCGAAGAGCCCCGUGUGCUCACUUUGAGUCCUCCGGCCCCUGAAUGUGGCUAACCUUAACCCU | |
| | GCAGCUAGUGCACACAAGCCAGUGUGUUGCUAGUCGUAAUGAGCAAUUGCGGGAUGGGACCAACUAC | |
| | UUUGGGUGUCCGUGUUUCACUUUUUACCUUUAUUUUGCUUAUGGUGACAAUAUAUAUAGUAUAUAUA | |
| | UUGGCACC | |
| 460 | UUAAAACAGCGGAUGGGUACCCCACCAUCCGACCCACUGGGUGUAGUACUCUGGUACUUCGUACCUU | rhinovirusB3 |
| | UGUACGCCUGUUCUUCCCAUUGUACCCUUCCUGAACUUCCAACCCAAGUAACGUUAGAAGCUCAACA | |
| | UUUAGUACAACAGGAAGCACCACAUCCAGUGGUGUUUAGUACAAGCACUUCUGUUUCCCCGGAGCGA | |
| | GGUAUAGGCUGUACCCACUGCCAAAAACCUUUAACCGUUAUCCGCCAACCAACUACGUAAAAGCUAG | |
| | UAGUAUUAUGUUUUUAACUAGGCGUUCGAUCAGGUGGAUUUCCCCUCCACUAGUUUGGUCGAUGAGG | |
| | CUAGGAAUUCCCCACGGGUGACCGUGUCCUAGCCUGCGUGGCGGCCAACCCAGCUUAUGCUGGGACG | |
| | CCUUUUUAUAGACAUGGUGUGAAGACUCGCAUGUGCUUGGUUGUGAUUCCUCCGGCCCCUGAAUGCG | |
| | GCUAACCUUAACCCUGGAGCCUUGUGUCACAAACCAGUGAUGAUAAGGUCGUAAUGAGCAAUUCCGG | |
| | GACGGGACCGACUACUUUGGGUGUCCGUGUUUCUUAUUUUUCUUAUUAUUGUCUUAUGGUCACAGCA | |
| | UAUAUAUAACAUAUACUGUGAUC | |
| 461 | UUAAAACAGCGGAUGGGUUUCCCACCAUCCGACCCACUGGGUGUAGUGCUCUGGUAUUUUGUACCUU | rhinovirusB4 |
| | UGCACGCCUGUUUCCCAUUUGUACCCUUCCUUAAUCUCCUUCCCCCGUAACGUUAGAAGUUUUGGAA | |
| | UUUUAAAGUACAAUAGGAAGCGCCACAUCCAGUGGUGUUGCGUACAAAUACUUCUGUUUACCCGGAG | |
| | CGAGGUAUAGGUUGUACCCACGGCCAAAAGCCUUUAACCGUUAUCCGCCAAUCAACUACGUAACGGC | |
| | UAGUAUCAUCUUGCUUUUGAUUUGGUGUUCGAUCAGGUGGUAUCCCCCACUAGUCUGGUCGAUGAGG | |
| | CUAGGAAUUCCCCACGGGCGACCGUGUCCUAGCCUGCGUGGCGGCCAGCCCAGCUUUUGCUGGGACG | |
| | CCUUUUCAAAGACAUGGUGUGAAGACCUGCAUGUGCUUGGUUGUGAGUCCUCCGGCCCCUGAAUGCG | |
| | GCUAACCUUAACCCUGGAGCCCAGCAGCAUAAUCCAAUGUUGUUUGGGUCGUAAUGAGCAAUUCCGG | |
| | GACGGGACCGACUACUUUGGGUGUCCGUGUUUCCUUUUAUUCUUACAUUGUCUUAUGGUCACAGCAU | |
| | AUAUAUUAUAUAUACUGUGAUC | |
| 462 | UUAAAACAGCGGGUGGGUAUCCCACCACCCGACCCACUGGGUGUGGUACACUGGUACUUUGUACCUU | rhinovirusB5 |
| | UGUAUGCCUGUUUGCUUCCACUCCCCUUUACCUAAUAUUCUUCCCCAAGUAUAUUUUGCGGUAACGU | |
| | UAGAAGAAGCAAAGUUAGUGCAAUAGGAUGUAUCACAACCAGUGGUAUAAAGCACAAGCAAUUCUUG | |
| | UUCCCCGGAGCUGGAUAUAGACUGCUAACGUGGUCAAAAGUCCUUAACCGUUAUCCGCCAGCUAACU | |
| | GCGAGAAGACUAGUAACACCAUGUUUGUUUUUAGACGUUCGAUCAGGUGAAUACCCCAUUCACUAGU | |
| | UUGGUCGAUGAGGCUGAGAAUACCCCACAGGUAACUGUGUCUCAGCCUGCGUGGCGGCCAACCCGAC | |
| | UCAUGUCGGGACGCCUACUAAUAGACAUGGUGUGAAGAUCCUAUUGCGCUUAGUUGUGAGUCCUCCG | |
| | GCCCCUGAAUGCGGCUAACCCUAACCCCGGAGCCUUGUGGUGUAAACCAACAUUUGCAAGGUCGUAA | |
| | UGAGUAAUUCUGGGAUGGGACCGACUACUUUGGGUGUCCGUGUUUCCUUUAUUUUUAUUGUGUGUGU | |
| | CUUAUGGUUACAAAUACAAUUUGUAAUC | |
| 463 | UUAAUACAGCGGAUGGGUAUCCCACCAUCCGACCCACUGGGUGUAGUACUCUGGUAUUAUGUACCUU | rhinovirusB6 |
| | UGUACGCCUGUUCUUCCCAUUGUACCCUUCCCAAAUUUCCAACCCAAGUAACGUUAGAAGUUUGACA | |
| | UUAAAGUACAAUAGGAGGCACCACAUCCAGUGGUGUUUUGUACAAGCACUUCUGUUUCCCCGGAGCG | |
| | AGGUAUAGGCUGUACCCACUGCCAAAAACCUUUAACCGUUACCCGCCAGCCAACUACGUAAAAGCUA | |
| | GUAACAUCUUGUUUGUAACUUGGCGUUCGAUCAGGUGGAUUCCCCCUCCACUAGUUUGGUCGAUGAG | |
| | GCUAGGAACUCCCCACGGGUGACCGUGUCCUAGCCUGCGUGGCGGCCAACCCAGCUCUUGCUGGGAC | |
| | GCCCUUUUAUAGACAUGGUGUGAAGACUCGCAUGUGCUUGGUUGUGAUUCCUCCGGCCCCUGAAUGC | |
| | GGCUAACCCUAACCCUGGAGCCUUGUGUUACAAACCAGUAAUAUUAAGGUCGUAAUGAGCAAUUCCG | |
| | GGACGGGACCGACUACUUUGGGUGUCCGUGUUUCUCAUUUUUCUUAUUAUUGUCUUAUGGUCACAGC | |
| | AUAUUUGUUAAAUAUACUGUGAUC | |
| 464 | UUAAAACAGCGGAUGGGUAUCCCACCAUUCGACCCAUUGGGUGUAGUACUCUGGUACUAUGUACCUU | rhinovirusB14 |
| | UGUACGCCUGUUUCUCCCCAACCACCCUUCCUUAAAAUUCCCACCCAUGAAACGUUAGAAGCUUGAC | |
| | AUUAAAGUACAAUAGGUGGCGCCAUAUCCAAUGGUGUCUAUGUACAAGCACUUCUGUUUCCCAGGAG | |
| | CGAGGUAUAGGCUGUACCCACUGCCAAAAGCCUUUAACCGUUAUCCGCCAACCAACUACGUAACAGU | |
| | UAGUACCAUCUUGUUCUUGACUGGACGUUCGAUCAGGUGGAUUUUCCCUCCACUAGUUUGGUCGAUG | |
| | AGGCUAGGAAUUCCCCACGGGUGACCGUGUCCUAGCCUGCGUGGCGGCCAACCCAGCUUAUGCUGGG | |
| | ACGCCCUUUUAAGGACAUGGUGUGAAGACUCGCAUGUGCUUGGUUGUGAGUCCUCCGGCCCCUGAAU | |
| | GCGGCUAACCUUAACCCUAGAGCCUUAUGCCACGAUCCAGUGGUUGUAAGGUCGUAAUGAGCAAUUC | |
| | CGGGACGGGACCGACUACUUUGGGUGUCCGUGUUUCUCAUUUUUCUUCAUAUUGUCUUAUGGUCACA | |
| | GCAUAUAUAUACAUAUACUGUGAUC | |
| 465 | UUUAAACAGCGGAUGGGUUCCCCACCAUCCGACCCACUGGGUGUAGUACUCUGGUAUUUUGUACCUU | rhinovirusB17 |
| | UGUACGCCUGUUUCUCCCCUACCUCCCAACCUAAACAAUCCUGGUAACUUAGAAGACUUAAAUCAUC | |
| | GUACAAUAGGUGCUGUCACAUCCAGUGACGGCUAGUACAAGCACUUCUGUUUCCCCGGAGCGGAGUA | |
| | UAAAUGGCCACCGCUGUCAAAAGCUCUUAACCGUUAUCCGCCAAUUAACUACGCAACGGCUAGUAAC | |
| | AUCUUGUUAUUUUUAGGGCGUUCGAUCAGGUGAGUAAACCCCUCACUAGUCUGGUCGAUGAGGCUGA | |
| | GAAUUCCCCACGGGCGACCGUGUCUCAGCCUGCGUGGCGGCCAGCCCAGCUAAUGCUGGGACGCCUU | |
| | AAUUGUGACAUGGUGUGAAGACCCACGUGUGCUUAAUUGUGAGUCCUCCGGCCCCUGAAUGCGGCUA | |
| | ACCUAAACCCUGGAGCCUUGAGACACAAUCCAGUGUUGGCAAGGUCGUAAUGAGUAAUUCCGGGACG | |
| | GGACCGACUACUUUGGGUGUCCGUGUUUCCUUUUAUUUUCAAAUUGUUCUUAUGGUCACAAUAUAAG | |
| | UAAUAUAUUGUGAUC | |
| 466 | UUAAAACAGCGGGUGGGUAUCCCACCACCCGACCCACUGGGUGUUGUACACUGGUAUUUUGUACCUU | rhinovirusB26 |
| | UGUAUGCCUGUUUGCACCUCCCCACCCCUUCCAAUUACCCUUACCCGAAUUaUAUUAUGCGGUAACA | |
| | UUAGAAGAAGUGAACACAGUGCAAUAGGACGUAUCACAUCCAGUGAUAUAAAGCACAAGCAAUUCUU | |
| | GUUCCCCGGAGCUGGAUAUAGACUGCUAACGUGGUUGAAGGUCCUUAACCGUUAUCCGCCAACCAAC | |
| | UACGAGACGGCUAGUAAUAUCAUGUUUGUCUUUGAGCGUUCGAUCAGGUGAAUUCCCCAUUCACUAG | |
| | UUUGGUCGAUGAGGCUGAGAACUCCCCACAGGUGACUGUGUCUCAGCCUGCGUGGCGGCCAACCCAG | |
| | CCACGGCUGGGACGCCCACUGAUAGACAUGGUGUGAAGACCCAAUUGUGCUUGGUUGUGACUCCUCC | |
| | GGCCCCUGAAUGCGGCUAACCUCAACCCCGGAGCCUUGUAGUGUAAGCCAACACAUACAAGGUCGUA | |
| | AUGGGCAACUCUGGGACGGGACCGACUACUUUGGGUGUCCGUGUUUCCUUUAUUUUUAUCUUUUGUG | |
| | UCUUAUGGUUACAAGUAUUGAUUGUAACC | |
| 467 | UUAAAACAGCGGAUGGGUUCCCUACCAUCCGACCCACUGGGUGUAGUACUCUGGUACUCUGUACCUU | rhinovirusB35 |
| | UGUACGCCUGUUUAUCCACUACCCUUUUCCUAAAUUUUCCACCCGUGUAACCUUAGAAGCAAACAAA | |
| | UUGUACAAUAGGGUGCAUCACAACCAGUGAUGUACAGUACAAGCACUUCUGUUUCCCCGGAGCGGGG | |
| | UAUAGACGGUCACCACUGUCAAAAGCCCUUAACCGUUACCCGCCAACCAACUACGUAACGGCUAGUA | |
| | ACACCAUGAAUUUGUCUUGACGUUCGAUCAGGUGGUAACCCCCCACUAGUUUGGUCGAUGAGGCUGA | |
| | GAACUCCCCACGGGUAACCGUGUCUCAGCCUGCGUGGCGGCCAACCCAGCUUCUGCUGGGACGCCAA | |
| | UUCAUUGACAUGGUGUGAAGACCUGCAUGUGCUUGGUUGUGAUUCCUCCGGCCCCUGAAUGCGGCUA | |
| | ACCCUAACCCCGGAGCCACGCGGCAUAAUCCAAUGUCGUAUGGGUCGUAAUGAGUAAUUCUGGGAUG | |
| | GGACCGACUACUUUGGGUGUCCGUGUUUCCUGUUUUUCCAAAUUAUUCUUAUGGUCACAGCAUAUAA | |
| | AUAGAUAUAUACUGUGAUC | |
| 468 | UUUAAACAGCGGAUGGGUAUCCCACCAUCCGACCCACUGGGUGUAGUACUCUGGUAUUUUGUACCUU | rhinovirusB37 |
| | UGUACGCCUGUUGUUCCUAAUGUACCCACCCUAAAACUUCCUACCCAAGUAACGUUAGAAGUUUCAU | |
| | CAACAAGUACAAUAGGAAGCAUCACAUCCAGUGGUGUUUUGUACAAGCACUUCUGUUUCCCCGGAGC | |
| | GAGGUAUAGGCUGUACCCACUGCCGAAAGCCUUUAACCGUUAUCCGCCAACCAACUACGUAAAAGCU | |
| | AGUAUCAUCAUGUUUUAAAAUAGGCGUUCGAUCAGGUGGAUCCCCCCUCCACUAGUUUGGUCGAUGA | |
| | GGCUAGGAACUCCCCACGGGUGACCGUGUCCUAGCCUGCGUGGCGGCCAACCCAGCUUCUGCUGGGA | |
| | CGCCUUUUUAUGGACAUGGUGUGAAGACUCGCAUGUGCUUGGUUGUGACUCCUCCGGCCCCUGAAUG | |
| | CGGCUAACCUUAACCCCGGAGCCCUGUGUUGCAAUCCAGUAACAUUAGGGUCGUAAUGAGCAAUUUC | |
| | GGGACGGGACCGACUACUUUGGGUGUCCGUGUUUCUCAUUUUUCUUAUUAUUGUCUUAUGGUCACAG | |
| | CAUAUAUAUAACGUAUAUACUGUGAUC | |
| 469 | UUAAAACAGCGGGUGGGUAUCCCACCACCCGACCCACUGGGUGUGGUACACUGGUACUUUGUACCUU | rhinovirusB42 |
| | UGUAUGCCUGUUUGCAUCCCCUUCCCUUUACCUAACAUCCCUCCCCAAGUUAUAUUUUGCGGUAACG | |
| | UUAGAAGAAGUGAACaUAGUGCAACAGGAAGUAUCACCACCAGUGAUAUAACGCACAAGCAAUUCUU | |
| | GUUCCCCGGAGCUGGAUAUAGACUGCUAACGUGGUUGAAAGUCCUUAACCGUUAUCCGCCAGUUAAC | |
| | UGCGAAAAGACUAGUAACAUCAUGCACAUUUCUAGGUGUUCGAUCAGGUGAAAACCCCAUUCACUAG | |
| | UUUGGUCGAUGAGGCUGAGAGUACCCCACAGGCGACUGUGUCUCAGCCUGCGUGGCGGCCAACCCGA | |
| | CUUAUGUCGGGACGCCUAUUAAUAGACAUGGUGUGAAGAUCCUAUUGCGCUUAGCUGUGAGUCCUCC | |
| | GGCCCCUGAAUGCGGCUAACCUUAACCCCGGAGUCUUGUGAUGUAAGCCAACAUUUGCAAGGUCGUA | |
| | AUGAGUAAUUCUGGGAUGGGACCGACUACUUUGGGUGUCCGUGUUUCCUUUAUUCUUAUCAUUUGUG | |
| | UUUCAUGGUUACAAGCAUUGUUUGUAAUC | |
| 470 | UUAAAACAGCGGAUGGGUAUCCCACCAUCCGACCCACUGGGUGUAGUACUCUGGUACUCUGUACCUU | rhinovirusB48 |
| | UGUACGCCUGUUUCUCCUCAACCCUCCCCUACCCGUUAUAUAUCUCAGUAACUUAGAAGAUAUCAUU | |
| | ACCGCACAAUAGGGACCGUUGUAACCAGCAACGGUCAGUGCAAGCACUUCUGUUUCCCCGGAGCGGA | |
| | GUAUAGACGGCCACUGCUGUCAAAAGCUCUUAACCGUUACCCGCCAGUUAACUACACAACAGCUAGU | |
| | AACAUCUUGUUGUUCUUAGGGCGUUCGAUCAGGUGAGUUUACCCCCUCACUAGUCUGGUCGAUGAGG | |
| | CUGAGAAAUCCCCACGGGUGACCGUGUCUCAGCCUGCGUGGCGGCCAGCCCAGCUUAUGCUGGGACG | |
| | CCUUAAGUAUGACAUGGUGUGAAGACUCGCGUGUGCUUAGCUGUGAGUCCUCCGGCCCCUGAAUGCG | |
| | GCUAACCUAAACCCCGGAGCCUUGAGGCACAAUCCAGUGCCCGCGAGGUCGUAACGAGAAAUUCUGG | |
| | GACGGGACCGACUACUUUGGGUGUCCGUGUUUCCUUUUAUCUUUUUGAUGUUCUUAUGGUUACAAUA | |
| | UAUAAAGUAUAUUGUAAUC | |
| 471 | UUAAAACAGCGGAUGGGUAUCCCACCAUCCGACCCACUGGGUGUAGUACUCUGGUAUUAUGUACCUU | rhinovirusB52 |
| | UGUACGCCUGUUUCUCCCCCCCAUUACCCCUCCCCACAUAUCCCAGUAACUUAGAAGAAUUAAAUUA | |
| | UCGCACAAUAGGAGCUAUUACAACCAGUAAUAGUUAGUGCAAGUACUUCUGUUUCCCCGGAGCGGAG | |
| | UAUAGAUGGCCACCGCUGUCAAAAGCUCUUAACCGUUAUCCGCCAGUUAACUACGUAACGGCUAGUA | |
| | ACAUCUUGUUGUGCUUAAGGUGUUCGAUCAGGUGAAGAGACACCCCCUUCACUAGUCUGGUCGAUGA | |
| | GGCUGAGAAAUCCCCACGGGCGACCGUGUCUCAGCCUGCGUGGCGGCCAGCCCAGCUUAUGCUGGGA | |
| | CGCCUUAAGUAUGACAUGGUGUGAAGACCCGCAUGUGCUUAACUGUGAGUCCUCCGGCCCCUGAAUG | |
| | CGGCUAACCUUAACCCUGGAGCCUUGGGGUACAAUCCAGUGCUAACAAGGUCGUAACGAGCAAUUCC | |
| | GGGACGGGACCGACUACUUUGGGUGUCCGUGUUUCCUUUUAUUUCAUUAUUGUUCUUAUGGUUACAA | |
| | UAUAUAGAGUAUAUUGUAAUC | |
| 472 | UUAAAACAGCGGAUGGGUAUCCCACCAUCCGACCCACUGGGUGUAGUACUCUGGUACUUUGUACCUU | rhinovirusB69 |
| | UGUACGCCUGUUUCUCCCUCCCUUCCCAACCUAAACAAUCCUUGUAACUUAGAAGGUUUCGAUUAUC | |
| | GCACAAUAGGGACUAUUGCAACCAGCAAUAGUCAGUGCAAGCACUUCUGUUUCCCCGGAGCGGAGUG | |
| | UAGAUGGCCACCGCUGUCAAAAGCUCUUAACCGUUAUCCGCCAGUUAACUACGCAACGGCUAGUAGU | |
| | GCCUUGUUGCUUUUAUGGCGUUCGAUCAGGCGAGAUUUACCCCCUCGCUAGUCUGGUCGAUGAGGCU | |
| | GAGAAUUCCCCACGGGCGACCGUGUCUCAGCCUGCGUGGCGGCCAGCCCAGCCUUUGCUGGGACGCC | |
| | UUGAUUGCGACAUGGUGUGAAGACCCGCGUGUGCUUAACUGUGAGUCCUCCGGCCCCUGAAUGCGGC | |
| | UAACCUAAACCCUGGAGCCUUGAAACACAACCCAGUGUUCGCAAGGUCGUAACGAGCAAUUCCGGGA | |
| | CGGGACCGACUACUUUGGGUGUCCGUGUUUCCUUUAUUCUUUCAAUUGUUCUUAUGGUCACAAUAUA | |
| | AGUGUUAUAUUGUGAUC | |
| 473 | UUAAAACAGCUGAUGGGUUCCCCACCAUCCGACCCAAUGGGUGUAGUACUCUGGUAUUCUGUACCUU | rhinovirusB70 |
| | UGUACGCCUGUUUCUCCCCACCCCCCCGUCCUAAAUAAUCCCAGUAACUUAGAAGAUUUGAAUUAUC | |
| | GUACAAUAGGUGCUGUCACAUCCAGUGAUGGCUAGUACAAGCACUUCUGUUUCCCCGGAGCGGAGUA | |
| | UAGAUGGCCACCGCUGUCAAAAGCUCUUAACCGUUAUCCGCCAAUUAACUACGCAACGGUUAGUAAC | |
| | AUCUUGUCAUUUUUGAGGCGUUCGAUCAGGUGAGUACACCCCUCACUAGUCUGGUCGAUGAGGCUGA | |
| | GAAUUCCCCACGGGCGACCGUGUCUCAGCCUGCGUGGCGGCCAGCCCAGCUUAUGCUGGGACGCCUU | |
| | AAUUGUGACAUGGUGCGAAGACCCACGUGUGCUUAAUUGUGAGUCCUCCGGCCCCCGAAUGCGGCUA | |
| | ACCUAAACCCCGGAGCCUUGAGACACAAUCCAGUGUUAGCAAGGUCGUAACGAGCAAUUCUGGGACG | |
| | GGACCGACUACUUUGGGUGUCCGUGUUUCCUGUUAUUCUCAAAUUGUUCUUAUGGUCACAAUAUAAG | |
| | UAAUAUAUAUAUUGUGAUC | |
| 474 | UUAAAACAGCGGAUGGGUUCUCCCACCAUCCGACCCACUGGGUGUAGUACUCUGGUAUGAUGUACCU | rhinovirusB72 |
| | UUGUACGCCUGUUUAUUCCCAAACCCCCACCCGUUAUUCGCCCAACCCCUGUAACGUUAGAAGUUUG | |
| | GAACUUAAUUGUACAAUAGGGAGCACCAUAUCCAAUGGUGUAACGUACAAGCACUUCUGUUUCCCCG | |
| | GAGCGAGGUAUAGGUGGUACCCACUGCCGAAAGCCUUUAACCGUCAUCCGCCAAUCAACUACGUAAC | |
| | AGCUAGUAACAUCUUGUUGUUGAUUGGACGUUCGAUCAGGUGGUUCACCCCCCACUAGUUUGGUCGA | |
| | UGAGGCUGGGAAUCCCCCACGGGUGACCGUGUCCCAGCCUGCGUGGCGGCCAACCCAGCUUAUGCUG | |
| | GGACGCCCUUUCAAUGACAUGGUGUGAAGACUCGCAUGUGCUUGAUUGUGAGUCCGCCGGCCCCUGA | |
| | AUGCGGCUAACCCUAACCCUGGAGCCUUGCACCACAAUCCAGUGGUGUCUGGGUCGUAAUGAGUAAU | |
| | UCUGGGAUGGGACCGACUACUUUGGGUGUCCGUGUUUCUCAUUUUUCUUUUAAUUGUCUUAUGGUCA | |
| | CAGCAUAUAGUAUUAUAUACUGUGAUC | |
| 475 | UUAAAACAGCGGACGGGUAUCCCACCAUCCGGCUCACUGAGCGUAGUACUCUGGUAUUGUGUACCUU | rhinovirusB79 |
| | UGUACGCCUGUUUUCCCCAAAUCCCCUACCCUUUAUAUCUCCUACCCCGUAACGUAAGAAGUUUUCA | |
| | AACAGUACAAUAGGAAGCACCACAUCCAGUGGUGUUAUGUACAAGCACUUCUGUUUCCCCGGAGCGA | |
| | GGUAUAGGUGGUCACCACCGCCAAAAGCCUUUAACCGUUACCCGCCAACCAACUACGUAAUGGCUAG | |
| | UAACAUCAUUGAUUUAGAUUGGCGUUCGAUCAGGUGGUAUCCCCCCACUAGUUUGGUCGAUGAGGCU | |
| | AGGAAUACCCCACGGGUGACCGUGUCCUAGCCUGCGUGGCGGCCAACCCAGCAUACGCUGGGACGCC | |
| | AGUUUACUGACAUGGUGCGAAGACUUGCAUGUGCUUGGUUGAGAGUCCUCCGGCCCCUGAAUGCGGC | |
| | UAACCUUAACCCCGGAGCCUCGCAGCAUAAUCCAAUGUUGUAGAGGUCGUAACGAGUAAUUCUGGGA | |
| | UGGGACCGACUACUUUGGGUGUCCGUGUUUCCUGUUUUUCUUUAAAAUGUCUUAUGGUCACAGCAUA | |
| | UAUACUGUAUAUACUGUGAUC | |
| 476 | UUAAAACAGCGGAUGGGUAUCCCACCAUCCGACCCACUGGGUGUAGUACUCUGGUAUUUUGUACCUU | rhinovirusB83 |
| | UGUACGCCUGUUUCUUCCCUUUGUACCCGCCCCUUAAACUUCCACCCCACGUAACGUUAGAAGUUUA | |
| | UACAAGCAGUACAAUAGGUUGCACCAUAACCAGUGGUGUUUCGUACAAGCACUUCUGUUUCCCCGGA | |
| | GCGGGGUAUAGACGGUACCCACUGUCAAAAGCCCUUAACCGUUAUCCGCCAAUCAACUACGUAAUGG | |
| | CUAGUAACACCAUGAAGGUAAGUUGACGUUCGAUCAGGUGGUAACCCCCACUAGUUUGGUCGAUGAG | |
| | GCUAGGAAUUCCCCACGGGUAACCGUGUCCUAGCCUGCGUGGCGGCCAACCCAGCAUUUGCUGGGAC | |
| | GCCAAUUUACUGACAUGGUGUGAAGACUUGCAUGUGCUUGAUUGUGACUCCUCCGGCCCCUGAAUGC | |
| | GGCUAACCUUAACCCCGGAGCCUUGCAGCAUAAUCCAAUGUUGUGGAGGUCGUAAUGAGCAAUUCUG | |
| | GGAUGGGACCGACUACUUUGGGUGUCCGUGUUUCCUGUUUUCUUUAAGUUGUCUUAUGGUCACAGCA | |
| | UAUAUACUAUAUAUACUGUGAUC | |
| 477 | UUAAAACAGCGGAUGGGUAUCCCACCAUCCGGCCCACUGGGCGUAGUACACUGGUAUUCUGUACCUU | rhinovirusB84 |
| | UGUACGCCUGUUUGCCGCCCCUCCCCCUUUUAUUACCACAUUUGUGGUCGCUGCAACGUUAGAAGAU | |
| | UUUAAAGUAGUGCAAUAGGAAGCAUCACAACCAGUGGUGUUAUGCACAAACACUUCUGUUUCCCCGG | |
| | AGCGAGGUAUAGACGGUCCCCACUGUCAAAAGCCUUUAACCGUUAUCCGCCAACCAACUACGAGACA | |
| | GCUAGUAAUGCCCUGUUCUCCUGCAAGGCGUUCGAUCAGACAGAUUUCCCCUCUGUUAGUCUGGUCG | |
| | AUGAGGCUAGAAGUACCCCACAGGUAACUGUGUUCUAGCCUGCGUGGCGGCCAGCCCAGCUUCUGCU | |
| | GGGACGCCUUUCAAGAGACAUGGUGUGAAGACUCAAUUGUGCUUGGUUGUGAGUCCUCCGGCCCCUG | |
| | AAUGCGGCUAACCUUAAACCCGGAUCCAUGCUAUGCAAACCAGCAUAGUUAUGGUCGUAAUGGGCAA | |
| | CUCUGGGAUGGGACCGACUACUUUGGGUGUCCGUGUUUCCAUUUUUACUUUUUGUGUCAUUUAUGGU | |
| | UACAAUUAAGUUGUAACC | |
| 478 | UUAAAACAGCGGAUGGGUACCCUACCAUCCGACCCACUGGGUGUAGUACACUGGUACAUUGUACCUU | rhinovirusB86 |
| | UGUACGCCUGUUUCUCCCUAUUGUACCUUCCCUCACAAUCCUAACCCCUGUAACGUUAGAAGAUGUG | |
| | CAACUCGUGCAAUAGGAGAUACUGCAACCAGUGGUAUCAAGCACAAGAACUUCUGUUUCCUCGGAGC | |
| | GGAGUAUAGAUGGUCCCCACCGUCAAAAGCUCUUAACCGUUAUCCGCCAAUCAACUACGUGACGACU | |
| | GGUAGCAUCAUUUUGACACUUUGACGUUCGAUCAGGUAGGCUUACCCCACCUACUAGUUUGGCCGAU | |
| | GAGGCUGGGAGUCCCUCACAGGUAACUGUGUCCCAGCCUGCGUGGCGGCCAACCCAGCAAUUGCUGG | |
| | GACGCCAAUUUGUUGACAUGGUGUGAAGAUCUUAAUGUGCUUGGUUGUGAGUCCUCCGGCCCCUGAA | |
| | UGCGGCUAACCUUAACCCCGGAGCCUUGUGUCACAAGCCAGUGACAUUAAGGUCGUAAUGAGUAAUU | |
| | UCGGGAUGGGACCGACUACUUUGGGUGUCCGUGUUUCCUGUUUUUCUUUAUGAUUGUCUUAUGGUCA | |
| | CAGCAUAUAGUAAUUAUAUACUGUGAUC | |
| 479 | UUAAAACAGCGGAUGGGUUUCCCACCAUCCGACCCACUGGGUGUAGUACUCUGGUACAUUGUACCUU | rhinovirusB91 |
| | UGUACGCCUGUUUCUCCCCUCCCUCCCAACCUAUACAAUCCCGGCAACUUAGAAGAUUUGAACUAUU | |
| | GUACAAUAGGCGCUGUCACAUCCAGUGGCGGCUAGUACAAGCACUUCUGUUUCCCCGGAGCGGAGUA | |
| | UAGAUGGCCACCGCUGUCAAAAGCUCUUAACCGUUAUCCGCCAAUUAACUACGCAACGGCUAGUAAU | |
| | AUCAUGUAACUUUUAGGGUGUUCGAUCAGGUGAGUGUACCCCUCACUAGUCUGGUCGAUGAGGCUGA | |
| | GAAUUCCCCACGGGCGACCGUGUCUCAGCCUGCGUGGCGGCCAGCCCAGCUUAUGCUGGGACGCCUU | |
| | AAUUGUGACAUGGUGUGAAGACCCACGUGUGCUUAAUUGUGAGUCCUCCGGCCCCUGAAUGCGGCUA | |
| | ACCUAAACCCUGGAGCCUUGAAACACAAUCCAGUGUUAGCAAGGUCGUAAUGAGUAAUUCCGGGACG | |
| | GGACCGACUACUUUGGGUGUCCGUGUUUCCUUUUAUUUUUAAGUUGUUCUUAUGGUCACAAUAUAAG | |
| | UAAUAUAUAUUGUGAUC | |
| 480 | UUAAAACAGUGGAUGGGUUUCCUACCAUCCGACCCACUGGGUGUAGUGCUCUGGUAUAUUGUACCUU | rhinovirusB93 |
| | UGCACGCCUGUUUUCCCAUUGUACCCUUCCUUAAAUUCCUCCCCAUGUAACGUUAGAAGUUUAAGAA | |
| | CAUAAAUGUACAAUAGGAAGCAUCACAUCCAGUGGUGUAUGGUACAAGCACUUCUGUUUAUCCGGAG | |
| | CGAGGUAUAAGCGGUACCCACUGCUGAAAGCCUUUAACCGUUAACCGCCAAUCAACUACGUAACGGC | |
| | UAGUAUUACCUUGUUUGCAAUCUGGCGUUCGAUCAGGUGGUUCCCCCCACUAGUUUGGUCGAUGAGG | |
| | CUAGGAGUUCCCCACGGGCGACCGUGUCCUAGCCUGCGUGGCGGCCAACCCAGCAUUUGCUGGGACG | |
| | CCUUUUUGUAGACAUGGUGUGAAGACCUGCAUGUGCUUGAUUGUGAGUCCUCCGGCCCCUGAAUGCG | |
| | GCUAACCUUAACCCCGGAGCCUUGUGACAUAAUCCAAUGUCAUUAAGGUCGUAAUGAGCAAUUCUGG | |
| | GACGGGACCGACUACUUUGGGUGUCCGUGUUUCUCUUUAUUCUUUACAUUGUCUUAUGGUCACAGCA | |
| | UAUAUAAUACAUAUAUACUGUGAUC | |
| 481 | UUAAAACAGCGGAUGGGUUUCCCACCAUCCGACCCACUGGGUGUAGUGCUCUGGUAUUUUGUACCUU | rhinovirusB97 |
| | UGCACGCCUGUUUCCCCUUUGUACCCAUCCUGAAUUUCCUCCCUCUGCAACGUUAGAAGUUUGUGAA | |
| | AUUAAAAGUACAAUAGGAAGCAUCACAUCCAGUGGUGUUCAGUACAAGCACUCCUGUUUCCCCGGAG | |
| | CGAGGUAUAGGUUGUACCCACGACCGAAAGCCUUUAACCGUUAUCCGCCAAUCAACUACAUAACGGC | |
| | UAGUAUCAUCAUGUUUUUGAUCUGGCGUUCGAUCAGGUGGUUUCCCCCACUAGUCUGGUCGAUGAGG | |
| | CUAGGAUUUCCCCACGGGCGACCGUGUCCUAGCCUGCGUGGCGGCCAGCCCAGCUUAUGCUGGGACG | |
| | CCUUUUUAAAGACAUGGUGUGAAGACCUGCAUGUGCUUGAUUGUGAGUCCUCCGGCCCCUGAAUGCG | |
| | GCUAACCUUAACCCUGGAGCCCGACAGCAUAAUCCAAUGUUGUUUGGGUCGUAAUGAGCAAUUCCGG | |
| | GAUGGGACCGACUACUUUGGGUGUCCGUGUUUCUUUUUAUUCUUAUAUUGUCUUAUGGUCACAGCAU | |
| | AUAUAGUAUAUAUACUGUGAUC | |
| 482 | UUAAAACAGCGGGUGGGUAUCCCACCACCCGACCCACUGGGUGUAGUACACUGGUAUUUUGUACCUU | rhinovirusB99 |
| | UGUAUGCCUGUUUGCCAUCCCUCCCUCUCCUCUUACCCUUACCCUUAUUUUGCGGUAACUUUAGAAG | |
| | UUUGUAAAUCACGCGCAAUAGGGUAUACCACAUCCAGUGGUAUGAAGCACAAGCAAUUCUUGUUCCC | |
| | CGGAGCUGGAUAUAAACUGCUAACGUGGUUGAAAGUCCUUAACCGUUAUCCGCCAGUUAACUGCGAG | |
| | AAGACUAGUAACUCCAUGUUUGUUUUUAAGCGUUCGAUCAGGUGGAUACCCCAUUCACUAGUUUGGU | |
| | CGAUGAGGCUGGGAAUCCCCCACAGGUAACUGUGUCUCAGCCUGCGUGGCGGCCAACCCAGCUUUUG | |
| | CUGGGACGCUUACUGACAGACAUGGUGUGAAGAUCCUAUUGCGCUUAACUGUGAGUCCUCCGGCCCC | |
| | UGAAUGCGGCUAACCUCAACCCCGGAGCCUUGUGGUGUAAACCAACACUCGCAAGGUCGUAACGAGU | |
| | AAUUCUGGGACGGGACCGACUACUUUGGGUGUCCGUGUUUCCUUUAUUUCUUAUUGAUUGUCUCCUA | |
| | UGGUUACAAAUUUACUGUAAUC | |
| 483 | UCCCCUCCACUAGUUUGGUCGAUGAGGCUAGGAAAUCCCCACGGGUGACCGUGUCCUAGCCUGCGUG | rhinovirusB101 |
| | GCGGCCAACCCAGCAUCAGCUGGGACGCCUUUUCAGAGACAUGGUGUGAAGACUCGCAUGUGCUUGA | |
| | UUGUGAUUCCUCCGGCCCCUGAAUGCGGCUAACCUCAACCCCGGAGCCUUGUAUUGCAAUCCAGCAA | |
| | UAUUAAGGUCGUAAUGAGCAAUUCUGGGACGGGACCGACUACUUUGGGUGUCCGUGUUUCCUGUUUU | |
| | UCAUAAAUUUUUCUUAUGGUCACAGCAUAUAGUACAUAUAUACUGUGAUC | |
| 484 | AAAACAGCGGAUGGGUAUCCCACCAUCCGACCCACUGGGUGUAGUACUCUGGUACUUUGUACCUUUG | rhinovirusB102 |
| | UACGCCUGUUCUUCCCAUUGUACCCUCCCUUAACUUCCAACCCAAGCAACAUUAGAAGUUAUCAAUU | |
| | UUCAGUACAAUAGGAAGCAUCACAUCCAGUGAUGUUGUGUACAAGCACUUCUGUCUCCCCGGAGCGA | |
| | GGUAUAAGCAGUACCCACUGCUAAAAGCCUUUAACCGUUACCCGCCAAUCAACUACGUAACAGCUAG | |
| | UAUCAUCAUGUUUUUGACUGGACGUUCGAUCAGGUGGUUCCCCCCCACUAGUUUGGUCGAUGAGGCU | |
| | AGGAAUACCCCACGGGUAACCGUGUCCUAGCCUGCGUGGCGGCCAACCCAGCAUUUGCUGGGACGCC | |
| | CUUUUAAAGACAUGGUGUGAAGACUCGCAUGUGCUUGAUUGUGAUUCCUCCGGCCCCUGAAUGCGGC | |
| | UAACCUUAACCCCGGAGCCUUGUGUUGCAAUCCAGCAAAAGCAAGGUCGUAAUGAGCAAUUCUGGGA | |
| | CGGGACCGACUACUUUGGGUGUCCGUGUUUCUCAUUUUACAUUACUUUUCUUAUGGUCACAGCAUAU | |
| | AUAAUAAAUAUACUGUGAUC | |
| 485 | UCUGGCGUUCGAUCAGGUGGAUUUCCCCUCCACUAGUUUGGUCGAUGAGGCUAGGAAUCCCUCACGG | rhinovirusB103 |
| | GCGACCGUGUCCUAGCCUGCGUGGCGGCCAACCCAGCAUAUGCUGGGACGCCCUUUUAUAGACAUGG | |
| | UGUGAAGACUCGCAUGUGCUUGGUUGUGAUUCCUCCGGCCCCUGAAUGCGGCUAACCUUAACCCUGG | |
| | AGCCUUGUAUUACAAACCAGUAAUAUUAAGGUCGUAAUGAGCAAUUCCGGGACGGGACCGACUACUU | |
| | UGGGUGUCCGUGUUUCUUAUUUUACUUAUUAUUGUCUUAUGGUCACAGCAUAUUUGUUAAAUAUACU | |
| | GUGAUC | |
| 486 | UUAAAACAGCGGAUGGGUAUCCCACCAUCCGACCCACUGGGUGUAGUACUCUGGUAUUAUGUACCUU | rhinovirusB104 |
| | UGUACGCCUGUUUCUCCCUCCCUACUACCCCGCCUCAAGCAUCCUGUAACUUAGAAGAAUUGAAAAA | |
| | CCGCACAACAGGAGCUAUUACAACCAGUAAUAGUUAGUGCAAGCACUUCUGUUUCCCCGGAGCGGAG | |
| | UAUAGACGGCCACCGCUGUCAAAAGCUCUUAACCGUUAUCCGCCAGUUAACUACGUAACGGCUAGUA | |
| | ACAUCUUGUUGUGCUUAGGGUGUUCGAUCAGGUGAAGAAAAACCCCCUUCACUAGUCUGGUCGAUGA | |
| | GGCUGAGAAGUCCCCACGGGCGACCGUGUCUCAGCCUGCGUGGCGGCCAGCCCAGCAUAUGCUGGGA | |
| | CGCCUUAAGUAUGACAUGGUGUGAAGACCCGCAUGUGCUUAACUGUGAGUCCUCCGGCCCCUGAAUG | |
| | CGGCUAACCUAAACCCUGGAGCCUUGGAGCACAAGCCAGUGCUUACAAGGUCGUAAUGAGCAAUUCC | |
| | GGGACGGGACCGACUACUUUGGGUGUCCGUGUUUCCUUUUAUCUCAUUAUUGUUCUUAUGGUUACAA | |
| | UAUAUAGAGUAUAUUGUAAUC | |
| 487 | GUGACAAUCAGCCAGAUUGUUAACGGUCAAGCACUUCUGUUUCCCCGGUACCCUUGUAUACGCUUCA | rhinovirusC1 |
| | CCCGAGGCGAAAAGUGAGGUUAUCGUUAUCCGCAAAGUGCCUACGAGAAGCCUAGUAGCACUUUUGA | |
| | AGCCUAUGGCUGGUCGCUCAACUGUUUACCCAGCAGUAGACCUGGCAGAUGAGGCUAGAUGUUCCCC | |
| | ACCAGCGAUGGUGAUCUAGCCUGCGUGGCUGCCUGCACACUCUAUUGAGUGUGAAGCCAGAAAGUGG | |
| | ACAAGGUGUGAAGAGCCUAUUGUGCUCACUUUGAGUCCUCCGGCCCCUGAAUGUGGCUAAUCCUAAC | |
| | CCCGUAGCUGUUGCAUGUAAUCCAACAUGUCUGCAGUCGUAAUGGGCAACUAUGGGAUGGAACCAAC | |
| | UACUUUGGGUGUCCGUGUUUCUUGUUUUUCUUUAUGCUUGCUUAUGGUGACAACUGUAGUUAUUACA | |
| | UUUGUUACC | |
| 488 | ACCGCACGCCAGUUUGCCACCCCCUACCCAUCGUAACUUAGAAGCUAUCAACUCGACCAAUAGGCGG | rhinovirusC2 |
| | UGGCAAACCAUACCACUUACGGUCAAGCACUCCUGUUUCCCCGGUAUGCGAGGAAUAGACUCCUACA | |
| | GGGUUGAAGCCUCAAGUAUCGUCAUCCGCAUUGGUACUACGCAAAGCUUAGUAGUGCCUUGAAAGUC | |
| | CCUUGGUUGGUCGCUCCGCUAGUUCCCCCUAGUAGACCUGGCAGAUGAGGCAGGACAUUCCCCACUG | |
| | GCGACAGUGGUCCUGCCUGCGUGGCUGCCUGCGCACCCCUAGGGGUGCGAAGCCAAGUGACGGACAA | |
| | GGUGUGAAGAGCCCCGUGUGCUACUAAUGAGUCCUCCGGCCCCUGAAUGCGGCUAAUCCAACCCCAC | |
| | AGCUAUUGCACACAAGCCAGUGUGUAUAUAGUCGUAAUGAGCAAUUGUGGGACGGAACCGACUACUU | |
| | UGGGUGUCCGUGUUUCCUUUUAUUCUUAUCAUUCUGCUUAUGGUGACAAUACUGUGAAAUAGUGUUG | |
| | UUACC | |
| 489 | UUAAAGCUGGAUCAUGGUUGUUCCCACCAUGAUUACCCACGCGGUGCAGUGGUCUUGUAUUACGGUA | rhinovirusC3 |
| | CAUUUCCAUACCAGUUUUAUACACCCCACCCCGAAACUCAUAGAAGUUUGUACACAAUGACCAAUAG | |
| | GUGGUGGCCAUCCAGGUCGCUAAUGGUCAAGCACUUCUGUUUCCCCGGCACCCUUGUAUACGCUUCA | |
| | CCCGAGGCGAAAAAUGAGGUUGUCGUUAUCCGCAAAGUGCCUACGAAAAGCCUAGUAACACUUUGAA | |
| | AACCCAUGGUUGGUCGCUCAGCUGUUUACCCAACAGUAGACCUGGCAGAUGAGGCUAGACAUUCCCC | |
| | ACCAGCGAUGGUGGUCUAGCCUGCGUGGCUGCCUGCACACCCUGCCGGGUGUGAAGCCAGAAAGUGG | |
| | ACAAGGUGUGAAGAGCCUAUUGUGCUCACUUUGAGUCCUCCGGCCCCUGAAUGUGGCUAACCCUAAC | |
| | CCCGUAGCUGUUGCAUGUAACCCAACAUGUAUGCAGUCGUAAUGGGCAACUAUGGGAUGGGACCAAC | |
| | UACUUUGGGUGUCCGUGUUUCCUGUUUUACUUUUUCAUUGCUUAUGGUGACAAUUGUAUCUGAUACA | |
| | CUUGUUACC | |
| 490 | UUAAAACUGGGCUCGGGUUGUACCCACCCGAGCUGCCCAUGUGGCGUGGUGCUCUUGUAUCCCGGUA | rhinovirusC4 |
| | CACUUGCACGCCAGUUUGCCUCUCCUUACCCCGUAACAUAUCUAGAAGAAUGUGCAUAACGGACCAA | |
| | UAGGUGGUGGCAACCCAUGCCACUGACGGUCAAGUACUUCUGUUUCCCCGGUGUGAUGUGGAAUAGA | |
| | CUCUCUCAGGGUUGAAGCCAUAAGCAUCGUUAUCCGCUACGUGCCUACGCAAAACCUAGUAGCAUUU | |
| | UGAAGCUUACUUGGUUGGCCGCUCAGCCACUAACCCGUGGUAGGCCUGGCAGAUGAGGCUGGGCGCA | |
| | CCCCACUGGCGACAGUGGCCCAGCCUGCGUGGCUGCCUGCCCCUCUUUGAGGGGAAGCCAUUUAAGU | |
| | GACAAGGUGUGAAGAGUCUCGUGUGCUCCACGUAGAAUCCUUCCGGCCCCUGAAUGUGGCUAAUCCU | |
| | AACCCCGCAGCUAUUGCACACAAUCCAGUGUGUUGAUAGUCGUAAUGGGCAACUGUGGGAUGGAACC | |
| | AACUACUUUGGGUGUCCGUGUUUCCUGUUUUCUUUAAAUACUUGCUUAUGGUGACAAAUAGUGUAUA | |
| | CUGUUUGACACC | |
| 491 | UUAAAACUGGGUACAGGUUGUUCCCACCUGUACCACCCAAGUGGUGUAGUGCUCUUGUAUUCCGGUA | rhinovirusC5 |
| | CAUUUGCACGCCAGUUUGCCACCCCUACCCUCUAUGUAACUUAGAAGCUUACACAAAGACCAAUAGG | |
| | CGGUGGUAAACCCAUACCACUAACGGUCAAACACUUCUGUUUCCCCGGCAUAUGAGGAAUAGGCUCC | |
| | AAAAGGGCUGAAGCCACUAGUGUCGUUAUCCGCAUUGGUACUACGCAAAGCCUAGUAUCGGCCUGAA | |
| | AAUUUCUUGGUUGGUCGUUCGGCCAGUUACCCCACUGGUAGACCUGGCAGAUGAGGCAGGACUUACC | |
| | CCACUGGCGACAGUGGUCCUGCCUGCGUGGCUGCCUGCACACCCUUAUGGGGUGUGAAGCCUAGGAA | |
| | CGGACAGGGUGUGAAGAGCCCCGUGUGCUACCAAUGAGUCCUCCGGCCCCUGAAUGCGGCUAAUCUU | |
| | ACCCCGCAGCUGUUGCACACAAUCCAGUGUGUAUGCAGUCGUAAUGAGCAAUUGUGGGAUGGAACCG | |
| | ACUACUUUGGGUGUCCGUGUUUCCUUGUUUUCCUUAUAUUUGCUUAUGGUGACAAUAUUGAUAUUAU | |
| | CAGUGUUGUCAUC | |
| 492 | UUGAAACUGAAUCAUGGUUGUUCCCACCAUGAUUACCCACGUGGUGCAGUGGUCUUGUAUUACGGUA | rhinovirusC6 |
| | CAUUUCCAUACCAGUUUUAUACACCCUACCCCGAAACAUAUAGAAGCUUGUACACAAUGACCAAUAG | |
| | GUGGUGACCAUCCAGGUUACCGACGGUCAAGCACUUCUGUUUCCCCGGUACCCUUGUAUACGCUUCA | |
| | CCAGAGGCGAAAAACGAGGUUAUCGUUAUCCGCAAAGUGCCUACGAGAAGCCUAGUAGCACUUUCGA | |
| | AACCUAUGGCUGGUCGCUCAGCUGCUCACCCAGCAGUAGACCUGGCAGAUGAGGCUAGACAUCCCCC | |
| | ACCAGUGAUGGUGGUCUAGCCUGCGUGGCUGCCUGCACACCCUAUCGGGUGUGAAGCCAGAAAGUGG | |
| | ACAAGGUGUGAAGAGCCUAUUGUGCUCACUUUGAGUCCUCCGGCCCCUGAAUGUGGCUAAUCCUAAC | |
| | CCCGUAGCUGUUGCAUGCAACCCAGCAUGUAUGCAGUCGUAAUGGGCAACUAUGGGAUGGAACCAAC | |
| | UACUUUGGGUGUCCGUGUUUCUUGUUUUACUUUUCAUUGCUUAUGGUGACAAUUGUAGUUGAUACAC | |
| | UUGUUACC | |
| 493 | UUAAAACUGGGUUAUAGUUGCUCCCACUAUAACCACCCACGCGGUGUUGUGGACUUGUAUUUCGGUA | rhinovirusC7 |
| | CACUUCCAUGCCAGUUUUAUCUUCCCCCCUGCAACACUUAGAAGAAAUGUACUUAUGGACCAAUAGG | |
| | CGGCGGCCACCCAGGUCGUUGACGGUCAAGCACUUCUGUCUCCCCGGUGAUACUGGAUACGCUUUAC | |
| | CCAAGGCGAAAACCGGGCUCAUCGUUAUCCGCAAAGUGCCUACGAGAAGUCUAGUAGCACUCUGAAA | |
| | ACCUAUGACUGGUCGCUCCACUGCAAUCCCAGCAGUAGACCUGGCAGAUGGGGCUAGACAUACCCCA | |
| | CCAGCGAUGGUGGUCUAGCCCGCGUGGCUGCCUGCACACCCUAUGGGUGUGAAGCCAGAAAGUGGAC | |
| | AGGGUGUGAAGAACCUAUUGUGCUCACUUUGAGUCCUCCGGCCCCUGAAUGUGGCUAAUCCUAACCC | |
| | UGCAACCGUUGCGCACAACCCAGUGCGUAUGCGGUCGUAAUGAGCAAUUGCGGGAUGGAACCAACUA | |
| | CUUUGGGUGUCCGUGUUUCUUGUUAUUCUUUUAUUUGCUUAUGGUGACACUGUAUAGCUAUACAUUG | |
| | UCAUC | |
| 494 | UUAAAACUGAGUAUGGGUUGUUCCCACCCAUACUGCCUACGGGCGUAGUACUCUUGUGCUCCGGUAC | rhinovirusC8 |
| | AUUUGUACGCCAGUUUUUCACCCCCACCCCCUAGGUAACUCUUAGAAGCACUUGCACACAGACGAAU | |
| | AGGCAGCCAACACUCCACGUUGGUCAACGUCAAGCACUUCUGUUUCCCCGGUAUUAGGGGUUAGGCU | |
| | UUUCAAGAGGCUGAAGCCUGAAGUAUCGUUACCCGCGCAAUUACUAAGCAGAGCCUAGUAUCACUUC | |
| | GACCCUUCCACUGGCUGGUCGUUCAAUGAGAAAACCCACUCAUAGACCUGGCAGAUGGGGCUGGAAA | |
| | UACCCCACUGGCGACAGUGUUCCAGCCCGCGUGGCUGCCUGUCCCCCAUUUUGGGGGAAAGCCAAUG | |
| | UAAGGACAAGGUGUGAAGAGCCGCGUUUGCUAAUUGUGAGUCCUCCGGCCCCUGAAUGCGGCUAAUC | |
| | UUACCCCACAGCUAUUGCAUGGAAGCCACCAUGUGGAUAGUCGUAACGGGCAACUGUGGGACGGAAC | |
| | CGACUACUUUGGGUGUCCGUGUUUCCUUUUAUUCCAUUUAUUAUCUCAUGGUGACAACGUAUACAGA | |
| | UUAUAUGUUGUUUACC | |
| 495 | UUAAAACAGCCGUGUGGUUGUACCCACCACCGGGCACAUUGUGCGUGGUACACUGGAAUUACGGUUC | rhinovirusC9 |
| | CUUUGUACGCCUGUUUUCCCUUCCCACUUUCUUUCUUCCUUCACUUCCUAAUUGUAACAUAAUUAGA | |
| | AGCAUGACAUACAGUCCAAUAGGAGGCGGUUAAUCCAUAACCGUCAUAGACAAGCACUUCUGUUUCC | |
| | CCGGAUGUGUGAAUAGGCUUCCACCGAGGCUGAAGCUGUAGCAUUCGUUAUCCGCACACGUACUACG | |
| | CGAAAACUAGUAACACCUUGAUUGGUGGUGUGGUGUUUCACUCGGCGCUACCCCAGCGUAGAUCAGG | |
| | GCAAUGGGGCUACGGAAACCCCACCGGCGACGGUGUCGUAGCCCGCGUGGUGCCCUGCCCAGAUCCU | |
| | UGCGAUCUGGGACACCAUAUCAUUGACAGGGUGUGAAGAUUCUAGUGUGCUACGUGUGAGUCCUCCG | |
| | GCCCCUGAAUGUGGCUAACCUUAACCCCGCAGCUAUGGUGUGCAAACCAGCACAUUCGUAGUCGUAA | |
| | UGGGCAACUGUGGGAUGGGACCAACUACUUUGGGUGUCCGUGUUUCCUUUUAUUCUUAUAAUUUUCC | |
| | UUAUGGUUACAAGCUUAUGGUUAUAGGUUUGUAACC | |
| 496 | CAUGGUUACCCACGAGGUGCAGUGGUCUUGUAUUACGGUACAUUUCCAUACCAGUUUUAUAAACCCU | rhinovirusC10 |
| | ACCCUGAAACUUAUAGAAGCUUGUACACACCGACCGACAGGUGGCAAUCAUCCAGAUUGUCGAAGGU | |
| | CAAGCACUUCUGUUUCCCCGGUAUCCUUGUAUACGCUUCACCCGAGGCGAAAACCGAGGUUAUCGUU | |
| | ACCCGCAAAGUGCCUACGAGAAGCCUAGUAUCACUUUGGAAACCUAUGGUUGGUCGCUCAGCUGUUU | |
| | UCCCAACAGUAGACCUGGCAGAUGAGGCUGGACGCUCCCCACCAGCGAUGGUGGUCCAGCCUGCGUG | |
| | GCUGCCUGCACACCCUGUGGGGUGUGAAGCCAGAAAGUGGACAAGGUGUGAAGAGCCUAUUGUGCUC | |
| | ACUUUGAGUCCUCCGGCCCCUGAAUGUGGCUAAUCCUAACCCCGUAGCUGUUGCAUGUAAUCCAACA | |
| | UGUAUGCAGUCGUAAUGGGCAACUAUGGGAUGGAACCAACUACUUUGGGUGUCCGUGUUUCUUGUUU | |
| | UUCUUUAUAUACUGCUUAUGGUGACAAUAGUAAUUCAUUACUCUUGUUACC | |
| 497 | UUAAAACUGGAUACAGGUUGUUCCCACCUGUAUCACCCAAGUGGUGUGGUGCUCUUGUAUUUCGGUA | rhinovirusC11 |
| | CGUUUGCACGCCAGUUUGCUACCCCUUCCCUUUUACGUAACUUAGAAGUUUACACAAAGACCAAUAG | |
| | GCGGUGGUAAAUCCAUACCACUAACGGUCAAAUACUUCUGUUUCCCCGGCAUGCGAGGAAUAGGCUC | |
| | CAAAAGGGCUGAAGCCACUAGUGUCGUUAUCCGCAUUGGUACUACGCAAAGCCUAGUAUUACCUUGA | |
| | AAAUUUCUUGGCUGGUCGCUCCACCAGAUACCCCACUGGUAGACCUGGCAGAUGAGGCAGGACUUAC | |
| | CCCACUGGCGACAGUGGUCCUGCCUGCGUGGCUGCCUGCACACCCCUUACGGGGUGUGAAGCCCAGA | |
| | AACAGACAAGGUGUGAAGAGCCCCGUGUGCUACUAGUGAGUCCUCCGGCCCCUGAAUGCGGCUAAUC | |
| | UUACCCCACAGCUGUUGCACGCAAACCAGCGUGUAUGCAGUCGUAAUGAGCAAUUGUGGGAUGGAAC | |
| | CGACUACUUUGGGUGUCCGUGUUUCUUUUUAUUCCUAUUAUUUGCUUAUGGUGACAAUAUUGAUAUU | |
| | AUCAGUGUUGUCAUC | |
| 498 | UUAAAACUGGGUAUAGGUUGUUCCCACCUGUACCACCCACGUGGUGUAGUGCUCCUGUAUUCCGGUA | rhinovirusC15 |
| | CACUUGCACGCCAGUUUGCCCUCCCUUCCUGUAACGUAGAAGCAAUGUAAACGGACCAAAAGGGGAC | |
| | GGCAAACCACGUCGUUCAAGGUCAAGCACUUCUGUUUCCCCGGCAUGCGAGGAAUAGACCUUCUCAA | |
| | GGGUUGAAGCCACAAGUGUCGUUACCCGCAUUGGUACUACACAGAGCCUAGUACUGCUCUAGAAACU | |
| | UAUUGGUUGGUCGUUCAGCCAUUUCCCCUGGUAGACCUGGCAGAUGAGGCAAGAAGUUCCCCACUGG | |
| | UAACAGUGUUCUUGCCUGCGUGGCUGCCUGCACACCCUAACGGGUGUGAAGCCAAUGAGUGGACAGG | |
| | GUGUGAAGAGCCGCGUGUGCUACCAAUGAGUCCUCCGGCCCCUGAAUGCGGCUAAUCCAACCCCACA | |
| | GCUAUGGCAUGCAAGCCAGCAUGUGCGUAGUCGUAAUGAGUAAUUGUGGGACGGAACCGACUACUUU | |
| | GGGUGUCCGUGUUUCCUUUAUUCUCUUAUGUUUUGCCUAUGGUGACAAUAUUAAGUAUUAAUAUUGC | |
| | UACC | |
| 499 | UGCACUUCUGUUUCCCCGGUACUAGGGUGUUAAACUUCCCCAGAGGUUGAAGCCCGAUGUAUCGUUA | rhinovirusC16 |
| | CCCGCAUGGUUACUAAGCAGAGCCUAGUAGCAUCUUGCUUUGCAUAUGAGCUGGUCGUUCAGUCAGU | |
| | AAACCCCCUGAUAGACCUGGCAGAUGAGGCUGGAAAUACCCCACUGGUAACAGUGUUCCAGCCUGCG | |
| | UGGCUGCCUGCCCCUCUUUUGAGGGGAAGCCCAUAUGUAGACAAGGUGUGAAGAGCCGCGUUUGCUA | |
| | ACUAUGAGUCCUCCGGCCCCUGAAUGCGGCUAAUCCAACCCCACAGCUACUGCGCAGAAACCACUGC | |
| | GUAGGUAGUCGUAAUGAGCAAUUGUGGGACGGAACCGACUACUUUGGGUGUCCGUGUUUCCUGUUUU | |
| | ACUUUACAUUGCUCAUGGACACAACAUAUAUAAAAUCGUAUAUGUUGUUCACC | |
| 500 | CACUUCUGUUUCCCCGGAUGUGUGAAUAGGCUUCACCCGAGGCUGAAACUGUAGCAUUCGUUAUCCG | rhinovirusC19 |
| | CAUGGCUACUACGCGACUGCUAGUAGCAUUCUGUAUGACAUGUGGGGUUUCGCUCAGCAGAGACCCA | |
| | GUCUGUAGAUCAGGGUAAUGAGGCUACACAAUCCCCACUGGCGACAGUGGUGUAGCCUGCGUGGUGC | |
| | CCUGCCCGAGGUUCGUCCUCGGGACCCUCCAUGUUAGACAGGGUGUGAAGACACUAGUGUGCUAGUC | |
| | AUGAGUCCUCCGGCCCCUGAAUGCGGCUAAUCUUAAACCUGCAGCCAUCGCAUGCAUGCCAGCAUGU | |
| | AGGUGGUCGUAAUGAGCAAUUGCGGGAUGGAACCGACUACUUUGGGUGUCCGUGUUUCCCUUUUUAC | |
| | AUUUAUGUGUCUCUUAUGGUUACAUCAUUGGUGUAAUC | |
| 501 | UUAAAACUGGGUUGUGGUUGUUCCCACCACAAUCACCUACGUGGUGUUGUAGUCUUGCAUUUCGGUG | rhinovirusC22 |
| | CAUUUCUAUGCCAGUUUUAUCUCCCCAGCCCCCGUAACUUAGAAGAAAAGAACACAGACCAAUAGGA | |
| | GGUGGUCACCCAGACCACUUACGGUCAAGCACUUCUGUUUCCCCGGUUGAUCUAGAUAUGCUUUACC | |
| | CGAGGCAAAAACUAAAUCAAUCGUUAUCCGCAAAGUGCCUACGGGAAGCCUAGUAGCACUCUGAAAG | |
| | CCUAUGGCUGGUCGCUCCAUUGCAAACCCAGCAAUAGACCUGGCAGAUGAGGCUAGACGUACCCCAC | |
| | CAGCGAUGGUGGUCUAGCCUGCGUGGCUGCCUGCACACCCUCAUCGGGUGUGAAGCCAGAAAGUGGA | |
| | CAGGGUGUGAAGAGCCUACUGUGCUCACUUUGAGUCCUCCGGCCCCUGAAUGUGGCUAAUCCUAACC | |
| | CUGCAGCUGUUGCACACAAGCCAGUGUGUAUGCAGUCGUAACGAGCAAUUGCGGGAUGGAACCAACU | |
| | ACUUUGGGUGUCCGUGUUUCUCUUUAUUCCUUAUGUUGACCUAUGGUGACAAGUGUGCAAUUCACGU | |
| | UUGUCACU | |
| 502 | UUAAAACUGGGCAGGAGUUGUUCCCACUCUUGCCACCCACAGGGUGUGGUGCUCUUGUAUUACGGUA | rhinovirusC23 |
| | CACUUGCAUGCCAGUUUUAUCUCCCCCCUCCCCACAAUGUAAUUUAGAAGCCAAUCACAUGACCAAU | |
| | AGGAAGUGGUAAGCCAUACCACUUAAGGUCAAGCACUUCUGUUUCCCCGGUAUGCGUGGAAUAGGCC | |
| | UCUUAAGGGGCUGAAGCCACAAUUAUCGUUACCCGCAUGUGAACUACGCGAAACCUAGUACUACCUU | |
| | GAAAGUACAUUGUCUGGUCGCUCAGCCAAUCCCCAUGGUAGACCUGGCAGAUGAGGCAAGAGGUACC | |
| | CCACUGGUAACAGUGUUCUUGCCUGCGUGGCUGCCUGCACACCCUUACCGGGUGUGAAGCCAAUGUA | |
| | UAGACAGGGUGAGAAGAGUCGAGUGUGCUUCACAUGAGUCCUCCGGCCCCUGAAUGCGGCUAAUCCA | |
| | ACCCCGCAGCUGUAGUAUGCAAGCCAGCAUAUGUGCAGUCGUAAUGGGUAACUGCGGGACGGAACCG | |
| | ACUACUUUGGGUGUCCGUGUUUCCUGUUUUUCCUUUAUUCAUGCUUAUGGUGACAAUAUUGAGAAUU | |
| | AAUAUUGUCACU | |
| 503 | AGCUCUGCAGAUAUCUCCCCGGACGUGUGAAUAGGCCCUACCAGGGCUGAAGCUGUAGCGUUCGUUA | rhinovirusC35 |
| | UCCGCAUGCCUACUGCGCAAAGGUUAGUAACUAACUAGAUACUUAGUGGUGUUUCGCUCAGCAGUAC | |
| | CCCACUGUAGAUCAGGGUAAUGAGGCUACGCAUCCCCCACUGGCGACAGUGGCGUAGCCUGCGUGGU | |
| | GCCCUGCCCAGGUUUAUACCUGGGACACCACUAUUUGGACAGGGUGUGAAGGCCAGCGUGCGCUAGG | |
| | CAUGAGUCCUCCGGCCCCUGAAUGCGGCUAAUCCUAACCCUGCAGCCAUUGCAUGCAAACCAGCAUG | |
| | UUGGUGGUCGUAAUGAGCAAUUGCGGGAUGGAACCGACUACUUUGGGUGUCCGUGUUUCUUGUUUUU | |
| | CCAUUAAUUGUCUUAUGGUUACAAGUUUGUGCAACAAAUUUGUGACC | |
| | UUAAAACUGAAUCAUGGUUGUUCCCACCAUGAUUGUCUACUGGACGUUGUACUCUUGUAUUCCGGUA | rhinovirusC39 |
| 504 | CACUUGUACGCCAGUUUUAUAACCCCUUCCCCUUUGCAACUUAGAAGAAAGUAAACACUGGCCAAUA | |
| | GGAAAUAGCAAACCACGCUAUUUACGGUCAAGCACUUCUGUUUCCCCGGUCUGACGGGAUAUGCUUC | |
| | ACCCGAGGCAGAAACUCAGAUGAUCGUUAUCCGCAAAGUGCCUACGGGAAGCCUAGUAGCACUUUGG | |
| | AAGUUGUUGGGCUGGUCGCUCAAUUGCUGACCCCGAGCAAUAGACUUGGCAGAUGAGGCUGGACGUG | |
| | CCCCACCAGUGAUGGUGGUCCAGCCUGCGUGGCUGCCUACUCACCCUAACAGGGUGAGAAGCCUAUC | |
| | AAUAGACAAGGUGUGAAGAGCCUACUGUGCUCACUUUGAUUCCUCCGGCCCCUGAAUGUGGCUAAUC | |
| | CUAACCCCGUAUCUAUCGCAUGCACACCAGCAUGUAGGUAGUCGUAAUGGGCAACUACGGGACGGAA | |
| | CCAACUACUUUGGGUGUCCGUGUUUCCUUGUUCUUUUAUUACUUGCGUAUGGUGACAAUUGUAUGAA | |
| | AUACGAUUGACACC | |
| 505 | GACCCGCACUAGUACUACGCAAAGCCUAGUAAUAUCUUGAAGCUCUUCUGGUUGGUCGUUCCACUAG | rhinovirusC40 |
| | UCACCCCCUAGUAGACCUGGCAGAUGAGGCAGGACACUCCCCACUGGUGACAGUGGUCCUGCCUGCG | |
| | UGGCUGCCUGCACACCCUCCGGGGUGUGAAGCCAAAAGAAGGACAAGGUGUGAAGAGCCCCGUGUGC | |
| | UACUAGUGAGUCCUCCGGCCCCUGAAUGCGGCUAAUCUUACCCCACAGCUAUUGCACACAAUCCAGU | |
| | GUGUAUGUAGUCGUAAUGAGUAAUUGUGGGACGGAACCGACUACUUUGGGUGUCCGUGUUUCCUUUU | |
| | AUUCUCAUGUUUUUGCUUAUGGUGACAAUACUGACAUAUAGUGUUGUUACC | |
| 506 | UUAAAACUGGGUAGGGGUUGUUCCCACCCCUACCACCCACGUGGUGUAGUGCUCCUGUAUUACGGUA | rhinovirusC41 |
| | CACUUGCACGCCAGUUUGCCUCCCUACCCCCUUCCCAUUCACACCCAACCCUGCGUAACUUAGAAGC | |
| | UAUGCACAAGACCAAUAGGUGGUAACAAUCCAUGUUACUAAAGGUCAAGUACUUCUGUUUCCCCGGC | |
| | AUGCGUGGAAUAAACAGUGCAAACGGUUGAGGCCACAAAUGUCGUUACCCGCGCAUGAACUACGCAG | |
| | AGCCUAGUAUCCCCCUGAAAGUCCAUUGGUUGGUCGCUCAGCUAAUCCCCAUAGUAGACCUGGCAGA | |
| | UGAGGCAAGACGCACCCCACUGGCGACAGUGGUCUUGCCUGCGUGGCUGCCUGCACACCCUUCGGGG | |
| | UGUGAAGCCAAUUGAUAGACAGGGUGUGAAGAGCCGAGUGUGCUUCAUGUGAGUCCUCCGGCCCCUG | |
| | AAUGCGGCUAAUCCAACCCCGCAGCUGUGGCACACAAUCCAGUGUGUAUGCAGUCGUAACGGGCAAC | |
| | UGCGGGACGGAACCGACUACUUUGGGUGUCCGUGUUUCCUUUUAUUCUUUAAUCAAGUACUUAUGGU | |
| | GACAAUAAUAAAUAUUGUUACC | |
| 507 | UUAAAACUGAGUGUGGGUUGUUCCCCACCCACACUGUCUACGGGACGUUGUACUCUUGUAUUCCGGU | rhinovirusC42 |
| | ACACUUGUACGCCCAGUUUGCCACCCUAAACACCCCGUAACAAUAGAAGUCUACGCACACUUAGACC | |
| | AAUAGGGGGUGUUCAUCCAGUUCACCCAAGGUCAAGCACUUCUGUUUCCCCGGCAUAGGGGUGUUAG | |
| | GCUUCGCCCGAGGCCAAAGCUCUGAUUGUCGUUAUCCGCACAACUACUGGACAAAGCCUAGUAGCAC | |
| | CCUACGAUGUAGCAUGGUUGGUUGUUCCGCCAGUAUCCCCUGGUAAACCUGGCAGAUGGGGCUAGAA | |
| | GUUCCUCACUGGCGACAGUGUUCUAGCCCGCGUGGCUGCCUGCUCCCUUAUGGGAGAAGCCAUUCUA | |
| | CUGACAAGGUGUGAAGAGCCCCGUACGCUAGUUGUGAUUCCUCCGGCCCCUGAAUGCGGCUAAUCCA | |
| | ACCCUGCAGCCAUUACAUGGAAGCCACCAUGUGGAUGGUCGUAAUGAGUAAUUGCGGGACGGAACCG | |
| | ACUACUUUGGGUGUCCGUGUUUCUUGUUUUUCUUUUACUGACGUUUAUGGUGACAAUAUAUAUAUAA | |
| | CGUAUAUAUUGUUCAUC | |
| 508 | GGUUGGUCGCUCCGUUGCAACCCCAGCAACAGACCUGGCAGAUGAGGCUGAGCAUUCCCCACCAGCG | rhinovirusC43 |
| | AUGGUGGCUCAGCCUGCGUGGCUGCCUGCGCACCCUUCGGGGUGCGAAGCCAGAAAGUGGACAAGGU | |
| | GUGAAGAGCCUAUUGUGCUCACUUUGAGUCCUCCGGCCCCUGAAUGUGGCUAAUCCUAACCCCGCAG | |
| | CUACUACACAUAAGCCAAUGUGCAGGUAGUCGUAACGGGUAACUGUGGGAUGGAACCAACUACUUUG | |
| | GGUGUCCGUGUUUCCUUGUUAUCCUUUUUUAAAUGCCUAUGGUGACAAUUGUACAGUAUACACUUGU | |
| | UACC | |
| 509 | GUGGUAAAUCCAUACCACUAAUGGUCAAACACUUCUGUUCCCCCGGCAUGUGAGGAAUAGGCUCCAA | rhinovirusC45 |
| | AAGGGCUAAAGCCACUAGUGUCGUUAUCCGCAUUGGUACUACGCAAAGCCUAGUAUCACUCUGGAAG | |
| | UCUCUCGGUUGGUCGCUCCACCAGGUACCCCACUGGUAGACCUGGCAGAUGAGGCAGGACUCACCCC | |
| | ACUGGCGACAGUGGUCCUGCCUGCGUGGCUGCCUGCACACCCCUACGGGGUGUGAAGCCUAGAGAUA | |
| | GACAGGGUGUGAAGAGCCCCGUGUGCUACCAAUGAGUCCUCCGGCCCCUGAAUGCGGCUAAUCUUAC | |
| | CCCACAGCUGUUGCACGCAAUCCAGCGUGUAUGCAGUCGUAAUGAGCAAUUGUGGGAUGGAACCGAC | |
| | UACUUUGGGUGUCCGUGUUUCCUUUUAUUCCUAUUAUUUGCUUAUGGUGACAAUAUUGAUAGCAUCA | |
| | GUGUUGUCAUC | |
| 510 | AACUGGGCUUGGGUUGUGCCCACCCAAGCUGCCCACGUGGCGUUGUGCUCUUGUAUUCCGGUACACU | rhinovirusC49 |
| | UGCACGCCAGUUUGCCUCCCCUACCCUGCAACGAAUUUAGAAGAAUGGACACAAAUAGACCAAUAGG | |
| | UGGUGACAAUCCAUGUCACUAAUGGUCAAGCACUUCUGUUUCCCCGGUGUGAUGUGGAAUAAACUCC | |
| | CCCAGGGUUGAAGCCAUAAGCAUCGUUACCCGCUACGUGCCUACGCAAAACCUAGUAGCAUUCUGGA | |
| | GUCUACUUGGUUGGCCGUUCAGCCACUAACCCGUGGUAGGCCUGGCAGAUGAGGCUGGGCGUACCCC | |
| | ACUGGUAACAGUGGCCCAGCCUGCGUGGCUGCCUGCCCCUCUUUGAGGGGAAGCCAAUUAGGUGACA | |
| | GGGUGUGAAGAGUCCCGUGUGCUCACGUAGAGUCCUCCGGCCCCUGAAUGUGGCUAAUCCUAACCCC | |
| | GCAGCUAUUGCACACAAUCCAGUGUGUUAGUAGUCGUAAUGGGCAACUGUGGGAUGGAACCAACUAC | |
| | UUUGGGUGUCCGUGUUUCUUGUUUUUCUUUUAUACUUGCUUAUGGUGACAAAUAGUGCAUACUGUUU | |
| | GACACC | |
| 511 | UUCCCCGGACGUGUGAAUAGGCUCCACCAGGGGCUGAAGCUGUAGCGUUCGUUACCCGCACUGCUAC | rhinovirusC50 |
| | UACGCAAGGGUUAGUAUCUAACUGAAAACAUUGUGGAGUUUCGCUCAGCAGAACCCCACUGUAGAUC | |
| | GGGGUAAUGGGGCUACACAACCCCCACCGGCGACGGUGGUGUAGCCCGCGUGGUGCCCCGCCUGAGG | |
| | CUUGUGCCUCAGGACGCCACAAUGUUGACAGGGUGAGAAGGCCUACGUGUGCUAGCAGUGAGUCCUC | |
| | CGGCCUCUGAAUGCGGCUAAUCCUAACCCCAUAGCCAUCACACACAAUCCAGUGUGCAGGUGGUCGU | |
| | AACGGGUAACUAUGGGAUGGAACCGACUACUUUGAGUGUCCGUGUUUCCUAUUUUACCUUUACAUGU | |
| | CUUAUGGUCACAAUAUAGUGUAUACAUAUUGUGAUC | |
| 512 | UUAAAACAGCCGUGUGGUUGUACCCACCACCGGACACACUGUGUGUUGUGCACUGGAAUCUCGGUUC | rhinovirusC51 |
| | CUUUGCAUGCCUGUUUUAAACCCCCCUUCCCCCACUCUUUCUAUCAUUCUUUUCUUCCUAAUGUAAC | |
| | UUAGACGUUAUACCACAGUCCAAUAGGACGUGGCCAAUCCAGGCCAUGACGGACAAGCACUUCUGUC | |
| | UCCCCGGGCGUGUGAAUAGGCCUCAACAAGGCUGAAGCUGUAGCGCUCGUUAUCCGCAUUUGUACUA | |
| | CGCGAUGGCUAGUAUCAUCCUUCUCUUGGUCUUGGCGUUUCGCUCAGCGUGGUAACCCCCCACGUAG | |
| | AUCAGGGCAAUGAGGCUGCAUAUGCCCCACCGGCGACGGUGAUGCAGCCUGCGUGGUGCCCCGCCUG | |
| | GGUUAACCCCCAGGACGCCACAAUCAAGACAGGGUGAGAAGACCUGAGUGUGCUACAGAUGAGUCCU | |
| | CCGGCCCCUGAAUGCGGCUAAUCCUAACCCUGCAGCUAUCGUAUGCAAACCAGCAUAUUGGUAGUCG | |
| | UAACGAGCAAUUGCGGGACGGAACCGACUACUUUGGGUGUCCGUGUUUCUCUUUUUAUUUGAAAUGU | |
| | UGCUUAUGGUUACAAGUUUUGGUAUUAAACUUGUGACC | |
| 513 | UUAAAACAGCUGUGUGGUUGUUCCCACCACCAGGCACACUGUGCGUUGUACACUGGGAUUCCGGUCA | rhinovirusC54 |
| | CUUUGUACGCCUGUUUGCUAUCCCCCCCAACUUAUGUAAUUUAGAAGAUGUACACAACGCCCAUUAG | |
| | GAUGCGGCCAAACCAGGUCCGCUUAGGGCAAGCACUUCUGUUUCCCCGGGUGUGUGAAUAGACUCUA | |
| | ACAGGGUUGAAGCUGUAGCACUCGUUAUCCGCGCAACUACUACGCGAAUGUUAGUAGCAUCCUGUGU | |
| | UGCAUUUGGGAUUUCGCUCCGCAGAAAACCCCAUCUGUAGAUUAGGGCAAUGAGGCUACACAUACCC | |
| | CACUGGCGACAGUGGUGUAGCCUGCGUGGUGCCCUACCCAGGCCUUCUUGGCCUGGGAUUCCACUUA | |
| | CAAGACAGGGUGUGAAGGCACUAGUGUGCUAGUUGUGAGUCCUCCGGCCCCUGAAUGCGGCUAAUCU | |
| | UAACCCCGUAGCCCCCGCAAGUAAACCAACUUGUAGGUGGUCGUAAUGAGUAAUUACGGGAUGGAAC | |
| | CGACUACUUUGGGUGUCCGUGUUUCCUUUUAUUCUUUAUAUUUGCAUCCUAUGGUUACAACAUAAGU | |
| | AAUC | |
| 514 | GCACUUCUGUUUCCCCGGACGUGUGAAUAGGCCUCACCAAGGCUGAAGCUGUAGCGUUCGUUAUCCG | rhinovirusC55 |
| | CUCACCUACUGCGCAAAGACUAGUAACUAACUAGAUACUUAGUGGUGUUUCGCUCAGCAGUACCCCA | |
| | CUGUAGAUCGGGGCAAUGAGGCUACGCAUCCCCCACUGGCGACAGUGGCGUAGCCUGCGUGGUGCCC | |
| | UGCCCAGGCUUCGGUCUGGGACACCACUAUGUUGACAGGGUGUGAAGACCGGCGUGCGCUAGGUGUG | |
| | ACUCCUCCGGCCCCUGAAUGCGGCUAAUCCUAACCCCGCAGCCACCGCAUGCAACCCAGCAUGUAGA | |
| | UGGUCGUAACGAGUAAUUGUGGGAUGGAACCGACUACUUUGGGUGUCCGUGUUUCCUGUUUUUCUUU | |
| | AUUGUUGUCCUAUGGUUACAAAUUUGUGUAUCAGAUUUGUGACC | |
| 515 | UUAAAACUGAACCAUGGUUGUUCCCACCAUGGUUACCCACGUGGUGCAGUGGACUUGUAUUACGGUA | rhinovirusC57 |
| | CAUUUCCAUACCAGUUUUAUAACCCCUACCCUGAAACUUUUAGAAGCAUGUACACAUACAGACCAAC | |
| | AGGUGGCAAUCAGCCAGAUUGCUAACGGUCAAGCACUUCUGUUUCCCCGGUAAUCUUGCAUACGCUU | |
| | CACCCGAGGCGAAAAGUGAGAUUAUCGUUAUCCGCAAAGUGCCUACGAGAAGCCCAGUAGCACAUUG | |
| | AAAGCCUAUGGUUGGUCGCUCAGCUGUUCACCCAACAGUAGACCUGGCAGAUGAGGCUAGAUAUUCC | |
| | CCACCAGCGAUGGUGAUCUAGCCUGCGUGGCUGCCUGCACACCCUAUCGGGUGUGAAGCCAGAAAGU | |
| | GGACAAGGUGUGAAGAGCCUAUUGUGCUCACUUUGAGUCCUCCGGCCCCUGAAUGUGGCUAAUCCUA | |
| | ACCCCGUAGCUGUUGCAUGUAACCCAACAUGUCUGCAGUCGUAAUGGGCAACUACGGGAUGGAACCA | |
| | ACUACUUUGGGUGUCCGUGUUUCUUGUUUUUCUUUAUAUUUGCUUAUGGUGACAAUUGUAGAUACUA | |
| | CACUUGUUACC | |
| 516 | ACCCUACUUAGGCAACCCCCACCCCUUCUCUUACGGGAGCUAGGGCCAAGGAUCUAUUGUAGUGGUG | simiansapelovirus3 |
| | UUGUAUCCCCUUACCUUAUACCCAUCCCCACCCAAACGGACUGGUAUUACGGUUUAGACUGAAUAAU | |
| | GGUUUAUGAAUUCAAACCUGUUACUCAUUCAGGGGAAAAAUGAAGACAUAUUCUAUGGAUUGGACCU | |
| | GUUAUGACCAAGAGUUGGCGUCUUGCGCGCUAGCCGUAUGGACAGUUUAAUGGUUACUCUUUGGAUU | |
| | AUGAUUUCAGGGUUGGCCGCCCAGGAUAAAAGGCAAUUGUGGCAAGUGAUGUUAGUCAUUGGCAGUU | |
| | GAAACCUGCCUAGAACAUCCUAGGUCUACGCUGUGCGGGCCGAAGUAAGUCUAGGAACAACAGGGAG | |
| | UAUGCUGUUUUUUGCUUUCACCCAACACGACCGUACACGAAAGAGCUAGAGGCAUCUGGAGGCAAAG | |
| | GGAAAAGAAUUUCUUAGCCCGAAUGUUCAUUAGAGUCCUUGACGAAUGCGUCCCCUCUGUCCCGAGG | |
| | GUGAGGCGUAUGGCGCAUGCUCAUGGCAUUACUCCAAGGUGUAUCUAGAGGGGGGGGCCCCCUCUAC | |
| | UUAGUCUAGUGCUACCUGACAGGGCCGCGGCUGGUCGUUUGUGUAUGGUAUAACCAGUAGUAAUCCC | |
| | CCAUGGAUUGCUUUAAAACCCCCUCCCCCCUCAACAAGACAUUCUCUAAG | |
| 517 | CCGACCAGUAGUGUCCGAAAUGCUAGAGUGGACUUCCUCCCUUCCCCUCUGGGUAUCUUGCCCAGUU | aichivirusA2; |
| | UCCUCCCCCCAACCUGACGUGCCACAGGCUGUGAAAACACCCCGCGAAAGCUGCCAAAAGUGGGAAU | caninekobuvirus |
| | UGUGGGUCCCCCCCUUUGCCAAGGAGUCGAGUCUUUCUCCCUUAAGGCUAGCCCCAUCAGUGAAUUC | |
| | UGUUGGGCAACUAGUGACGCCACUGCAUGCCUCCGACCUCGGCCGCGGAGUGCUGCCCCCCAAGUCG | |
| | UGCCCCUGACCACAAGUUGUGCUGUCUGGCAAACAUUGUCUGUGAGAAUGUUCCGCUGUGGCUGCCA | |
| | AGCCUGGUAACAGGCUGCCCCAGUGUGCGUAGUUCUCAUCCAGACUUCGGUCUGGCAACUUGCUGUU | |
| | AAGACACGGCGUAAGGGGCGUGUGCCAACGCCCUGGAACGAGUGUCCACUCUAAUACCCCGAGGAAU | |
| | GCUACGUAGGUACCCCUGGUUCGCCAGGGAUCUGAGCGUAGGCUAAUUGUCUAAGGGUAUUUUCAUU | |
| | UCCCACUCUCUCUUUCUUGUUCAUA | |
| 518 | GGAAUUAAACGGGCACCCACUCACCCCCAUCCCCCCCUGUAACUUAAAGUGUGUGCUCGUGACCUUG | aichivirusA4; |
| | ACUCCUGCGGGCUUCACCGACCCGUCCAGUGAACAAACUGCUAGGUGACUCCUUCCCCCCCCCUGGC | felinekobuvirus |
| | CUGGAUGGCCCACAUCCAGGUCUUCUUCCCCCAGCCCGACAUGUCAUGGGCUGUUUGGAAUCCCCGC | |
| | GAAAGCUGCUAACGUGGCAAUUGUGGGUCCCCCUUCCAACAAGACACCGAGUCUUUCUCCCUUAAGG | |
| | CUAGCCCCGUGUGGUGAAUUCCACCGGGCAACUAGUGGUGUCAAUGCAUGCCUCCGACCUCGGCCGC | |
| | GGAGUGCUGCCCCCCAAGUUGCGAAACUGACCAAGACUGUGCUGCCUGGCAAGCAUAGUCUGAGGAG | |
| | AUGUUCCGCUGUGGUUGCCAAACCUGGUAACAGGUGCCCCAGUGUGCGUAGUCUCCUUCCGGUUCGU | |
| | CCGGCUGUCCUUUGGUAAAGACACAAUGUAAGGGGCGUGUGCCAACGCCCUGGAAAGAGUGAACUUC | |
| | UACUGCCCUAGGAAUGCCACGCAGGUACCCCUGGUUUCCCACCAGGGAUCUGAGCGUGGGCUAAUUG | |
| | UCUAACGGGUAGUUUCAUUUCCCUCCUCUCUUUUGUUGACAUC | |
| 519 | UUCGGUACCUGUUUGGAGGAAUUAAACGGGCACCCUACCCAAGGGUUACAUGGGACUCCAACUCCCC | aichivirusA7 |
| | UUUCCCCGUAACUCUAAGUAAUGUGCUCGUAAUCUUGACCCCUCCGGGUUGUGUCGCCCCGUGAGUG | |
| | AACAAAUAGUUAGACACGCUCCUCCCUUCCCCUGGGUGGGCACACGCCCACUCACAUCCACCCCCCA | |
| | GCACAACAUGUUCGUGCUGUGGUUGAUGCACCUGGAAGCCGCGAAAGCUUCGGUUAGUAGUGGCAAC | |
| | CGGUUGACCUACACAACCAAAUGCUCAGAGUCUUUCUCCCUUAAGGCUAGACCCCCUGGUGAAUUCC | |
| | ACGGUGGCAACUAGUUGAGCAAACAUGUCCCACAAGUGCGGCCGCGGAGUGCUGUUCCCCAAGCCUU | |
| | GUGUGUCCUCCUGUGAAGUUGUGCCUGGCAAGCAUAUCUGAGAAGAUGUUCCGCUGCGUCGUGCCAA | |
| | CCUGGCAACAGGUGACCCAGUGUGCGUAGUCUUCUUCCGUCUUACGACGGACUUCCUCUAGGAUGCC | |
| | CUGGUGUAAGGUACAUGUGCCAACGCCCCAUGCGUGGUGUUCUUCUAAGGCCCUAGGAAUGCACCGC | |
| | AGGUACCCCUGUCUCGGCAGGGAUCUGAGCGGUGGCUAAUUGUCUACGGGCCUUUUCAUUUCCCAUU | |
| | CUUCUUGACUGUCAUCAUGGCCACAUCACACGGAUCUCGUUUGGUGUCCAAUGCUUCUCAGCUUCUU | |
| | UCCUCCUAUGAUAGACUCUACAGAACUGUCAGGGACUGGCGUGACACUGAUCAACUGCGCCAGACCC | |
| | GAAUGCCCUACCUUCUUCCUGUUGCCCCUGGUGAGUGCAUUUCCUGCUACCUUGAGCGGACCGAACG | |
| | GGACCCCAUGUCCAUCACGGACGAUGGGUACCCCUGGACGGUUUGCCCAGACUGCAAGACCCCUCCC | |
| | AAACGCAACCCCCCCCCCCCGAAGAGUUUCUGUAUGACUCUGACGUCCAGUCUUGGUUCGAAGAAGA | |
| 520 | CCAGACUCCUGUGACUGACAGAGAACAGAACAUUUUUGACCAAAUAGCC | |
| | GCCAGGUCGCAUCGCCUGGUUGGUGACCAAACUGCUAGAUGCUCCUCCCUUUCCCUCGGGGUUGGCG | aichivirusA8; |
| | CACCGCCAUCCCUGCAUCCUCCCCCCAGCCUGACAUGCUACAGGCUGUGCAAGAACCCCGCGAAAGC | murinekobuvirus |
| | UGCCACCGUGGCAAUUGUGGGUCCCCCUCUUGUCAAGGCACAGAGUCUUUCUCCCUUAAGGCUAGAC | |
| | CCCUCUGUGAAUUCAGAGGCGGCAACUAGUUGUGCCACUACAUGCCUCAGACCUCGGUCGCGGAGUG | |
| | CUGUUCCCCAAACCCUGACACUGCCGCGUGAGUAUGCUGCUUGGCAAGCAUUGCCUGGAUGGUCGUU | |
| | CCGCUGGGGCUGCCAGGUCUGGUAACAGACCGCUCCAGUGUGCGUAGACCAUCUCCAGCUUCGGCUG | |
| | GUGCUCACAAGGUAAGACUCGGUGUAAGGUUCAUGUGCCAACACCCUGGAAUGAGUGAAAACUCUAC | |
| | UGCCCUAGGAAUGCUACGCAGGUACCCCUGCUCCCUUGGGAGCCAGGGAUCUGAGCGUAGGCUAAUU | |
| | GUCUGUCGGGUAGUUUCAUUUCCCACCCUCUAGACUGUUCACC | |
| 521 | UUUGAAAAGGGGGUGGGGGGGCUUCGGCCCCCUCACCCUCUUUUCCGGCGGCGCAUGUUCGCGUCGU | aichivirusB1; |
| | CGUAAGUCUGGAUUCCCAAGGCCCACUCGGUUCAACUUCGGUUUCCGGACAAAUACAAAGAACCUCA | bovinekobuvirus |
| | GUCCUCUUGGUACUUUCUCGCCUGAGUCUACAAAGCGAGAAACCUGCCCCUCUAACGCCAGACGAGC | |
| | GGCAUAAAACUCGAACUUCUGGCACGUUCCACCACCCCUUCUCCUAUCCCAACCCCCAUUGCGCUCU | |
| | CAAGGUCGCGCUUUUCCGAGACUAGCUCGGAUUCAAAAAGUUCCUGGCACCCUUUACCCCUUCAGGC | |
| | CCUUAAGGUAGGAACUGACCUUGUGCUGUGAUCUCGGUGCGGAAGUGCUACUGCGUAGUGAUUGUAA | |
| | GAUCCUUUUGUGGUUCUGCCCUGGCAAGGCUACAGAGUGCUGUGAUCCGCUGCGGAUGCCAUCCUGG | |
| | UAACAGGACCCCCAGUGUGCGCAACAGUAUGUUCACGGUCUUCCGUGUUCACCACAUUCGGAACACU | |
| | GCUUUCGUGAAACAGUGUGUGUCCAAUCCCUGUGAUCAGUAUCAACCACACCACCUAGGAAUGCUAG | |
| | GAAGGUACCCCGGUUCGCCGGGAUCUGAUCCUAGGCUAAUUGUCUACGGUGGUGCUCCUUUUUAUUU | |
| | UCCACUUCAACUCACUGGUUACAACUGCUUGAUUUCUGUGUUUGCUGCUUUUCUCUGCUCUCACUGC | |
| | CAUUCUCAAGUGUUCACACUGUCCAAGCUCCUUUGGUUGUUCGCUUCCACUUGCCACUGUCAACUCU | |
| | UGUC | |
| 522 | CCAUGGUGUACAUCGGUUUCCGGGCAAAUACAAGAACCCCAGUUUUCCUGGCAAUUCCUUGACUGGG | aichivirusB2; |
| | UUAAAGAAAUAAGGAACCAGCCCCUCUAACGCCAGACGAGCGGCUUUAAACUUGAACUUCUGGCACA | ferretkobuvirus |
| | CUUUUCUCCACUACCCUUUUCCCCAAACUCGGACUGACUCACUGGAUUUUGACCACUCCCCCUUUGU | |
| | CUGACCACUCUUUUGGAUCUCCCUCCAACAUGCCGGUGGUGCCUGGCGGAAAAAGCACAAGUUGUGU | |
| | GAGCAACACCCCCACAGGUCCCACGGAACGGUCUGUGCCCGCAGAUCUAGUACGUGCGGAUCCCUGG | |
| | UUGCUUCCACUGCUCGCCUGUGAGUAGAGUAGGUGCUGGGUGUGAUGCGUCUUCGGACGCCUCGGGC | |
| | AUAGCAUUGCUCCCCCGAGAAUCAAGCCAUCGAGAUUUCUCUGGCCUCUGAAGUCACGCCUGGACGC | |
| | GUAG | |
| 523 | UUUGAAAAGGGGGUGGGGGGGCCUCGGCCCCCUCACCCUCUUUUCCGGUGGCCACCCGCCCGGGCCA | aichivirusC1; |
| | CCGUUACUCCACUCCACUCCUUCGGGACUGGUUUGGAGGAACAUAACAGGGCUUCCCAUCCCUGUUU | porcinekobuvirus |
| | ACCCUUACUCCACUCACCCUUCCCCUUGACCAACCCUAUCCACACCCCACUGACUGACUCCUUUGGA | |
| | UCUUGACCUCGGAAUGCCUACUUGACCUCCCACUUGCCUCUCCCUUUUCGGAUUGCCGGUGGUGCCU | |
| | GGCGGAAAAAGCACAAGUGUGUUGUUGGCUACCAAACUCCUACCCGACAAAGGUGCGUGUCCGCGUG | |
| | CUGAGUAAUGGGAUAGGAGAUGCCAAUAACAGGCUCGCCCAUGAGUAGAGCAUGGACUGCGGUGCAU | |
| | GUGACUUCGGUCACCAGGGGCAUAGCAUUGCUCACCCCUGAAUCAAGUCAUCGAGAUUUCUCUGACC | |
| | UCUGAAGUGCACUGUGGUUGCGUGGCUGGGAAUCCACGCUUGACCAUGUACUGCUUGAUAGAGUCGC | |
| | GGCUGGCCGACUCAUGGGUUAAAGUCAGUUGACAAGACAC | |
| 524 | CAUCCCCAAUAUGUUCGGUGAGGAAUGCUACCACGUAACUAUUAGUGAACCCAUUGACUGUUCUGCC | aichivirusD1; |
| | CUGGCAAGGCUAUAGAGUACAGUGUUCCGCUGUGGAUGCCGUCCUGGUAACAGGAUCCCCAGUGUGU | kagovirus1 |
| | GUAGCUGUAUGUUCAACUUCGGUUGUUCACAGUCCUUGGUCUAGCUUUUAGUGAAAUGGUGUGCGCC | |
| | AUAUCCCUACAUUUGGGUGCCACCUCUCAACCCCUAGGAAUGCUAGGAAGGUACCCCACCCUCCGGU | |
| | GGGAUCUGAUCCUAGGCUAAUUGUCUAUGGGGUUGUUUCCUUUUCUUUUCACCACUCAUUACUGGCU | |
| | CACAGAUUGUUUGUUGUGUUCCCUGCUCUCUCUAGCUCCCAACACGGCACACAGUCUCAGCCACACC | |
| | GCCUUUGCAAACGACUACCAGAUUUGUUUCUUUGACUCCUCUGAUUCUCACAUCUUCACUGAACCAG | |
| | AC | |
| 525 | GAAAGGGGGCCCGGCGGGGCAACACCCGUCGGUCCCUCUUUCCGUCCAAGCCGCGGACGUUAAACUG | oscivirusA2; |
| | UGGCACGGUUGUGCCUAGGUGCAACCCUGCUACUAAUGGCGGUACCCCUGCCCUUAGUAUGCAUGUU | turdivirus3 |
| | UGUAUGAAUGGAAUCUGUAGUGAUGCCCUGCUUUGGGGUCUCUAAAACACACUACAGUGAUGGCUAG | |
| | AGCUCCUCUACUCCGGCUACCUUAGUAGUGAGCAUUGGAAGUAUAUUGUGAACAGGGUUCCACGCCC | |
| | GAUAAAGGCUGGUAUUCCCGCUCAUGGAGUGGACCUAGGUAACUAGGGCCAUGAGUGUGUGUGAAUC | |
| | AGCACCCGCAAGGGAUAGCAGUAUAUGGAAGGUGCUCGUGAAACGGCAAGUGGAGUUAGUAGCAGCU | |
| | CUCAGUCUCAAGGUCAGCUUCUAGACAUGUGCCGCAGGUACCCCGUAAGGGAUCUGAGCGGUAGCUA | |
| | AUUAGUCCCUGGAGGUUGGCUGACUUUCUUCCUAUUUCUCUUUUCACUGCGAAUUGUGUGGAUUGGU | |
| | AUUCUUUAGAGCUUGGGAGUUGUCUCAUAUUGAAUUCAUAUAUUUUACUCUAUACUACACUCAAACA | |
| | CGGCUCUCUGGUCAGUUAAGAAAAUCAUUUAUAGAAUAAUAAA | |
| 526 | UUAAAACAGCGGAUGGGUACCCCACCAUCCGACCCACUGGGUGUAGUACUCUGGUACUUCGUACCUU | HRV-B3 |
| | UGUACGCCUGUUCUUCCCAUUGUACCCUUCCUGAACUUCCAACCCAAGUAACGUUAGAAGCUCAACA | |
| | UUUAGUACAACAGGAAGCACCACAUCCAGUGGUGUUUAGUACAAGCACUUCUGUUUCCCCGGAGCGA | |
| | GGUAUAGGCUGUACCCACUGCCAAAAACCUUUAACCGUUAUCCGCCAACCAACUACGUAAAAGCUAG | |
| | UAGUAUUAUGUUUUUAACUAGGCGUUCGAUCAGGUGGAUUUCCCCUCCACUAGUUUGGUCGAUGAGG | |
| | CUAGGAAUUCCCCACGGGUGACCGUGUCCUAGCCUGCGUGGCGGCCAACCCAGCUUAUGCUGGGACG | |
| | CCUUUUUAUAGACAUGGUGUGAAGACUCGCAUGUGCUUGGUUGUGAUUCCUCCGGCCCCUGAAUGCG | |
| | GCUAACCUUAACCCUGGAGCCUUGUGUCACAAACCAGUGAUGAUAAGGUCGUAAUGAGCAAUUCCGG | |
| | GACGGGACCGACUACUUUGGGUGUCCGUGUUUCUUAUUUUUCUUAUUAUUGUCUUAUGGUCACAGCA | |
| | UAUAUAUAACAUAUACUGUGAUC | |
| 527 | GGGAGACCCUCGACCGUCGAUUGUCCACUGGUCAACAAUAGAUGACUUACAACUAAUCGGAAGGUGC | AnaX-CVB3- |
| | AGAGACUCGACGGGAGCUACCCUAACGUCAAGACGAGGGUAAAGAGAGAGUCCAAUUCUCAAAGCCA | IL2 |
| | AUAGGCAGUAGCGAAAGCUGCAAGAGAAUGAAAAUCCGUUGAUUAAAACAGCCUGUGGGUUGAUCCC | precursor |
| | ACCCACAGGCCCAUUGGGCGCUAGCACUCUGGUAUCACGGUACCUUUGUGCGCCUGUUUUAUACCCC | |
| | CUCCCCCAACUGUAACUUAGAAGUAACACACACCGAUCAACAGUCAGCGUGGCACACCAGCCACGUU | |
| | UUGAUCAAGCACUUCUGUUACCCCGGACUGAGUAUCAAUAGACUGCUCACGCGGUUGAAGGAGAAAG | |
| | CGUUCGUUAUCCGGCCAACUACUUCGAAAAACCUAGUAACACCGUGGAAGUUGCAGAGUGUUUCGCU | |
| | CAGCACUACCCCAGUGUAGAUCAGGUCGAUGAGUCACCGCAUUCCCCACGGGCGACCGUGGCGGUGG | |
| | CUGCGUUGGCGGCCUGCCCAUGGGGAAACCCAUGGGACGCUCUAAUACAGACAUGGUGCGAAGAGUC | |
| | UAUUGAGCUAGUUGGUAGUCCUCCGGCCCCUGAAUGCGGCUAAUCCUAACUGCGGAGCACACACCCU | |
| | CAAGCCAGAGGGCAGUGUGUCGUAACGGGCAACUCUGCAGCGGAACCGACUACUUUGGGUGUCCGUG | |
| | UUUCAUUUUAUUCCUAUACUGGCUGCUUAUGGUGACAAUUGAGAGAUCGUUACCAUAUAGCUAUUGG | |
| | AUUGGCCAUCCGGUGACUAAUAGAGCUAUUAUAUAUCCCUUUGUUGGGUUUAUACCACUUAGCUUGA | |
| | AAGAGGUUAAAACAUUACAAUUCAUUGUUAAGUUGAAUACAGCAAAAUGUAUAGAAUGCAGCUGCUG | |
| | UCUUGCAUCGCCCUGAGCCUGGCCCUGGUCACAAACAGCGCUCCUACAAGCUCUAGCACCAAGAAAA | |
| | CCCAGCUGCAGCUCGAGCACCUGCUGCUGGACCUGCAGAUGAUCCUGAACGGCAUUAACAACUACAA | |
| | GAACCCAAAGCUGACCAGAAUGCUGACAUUUAAGUUCUACAUGCCUAAGAAGGCCACAGAGCUGAAG | |
| | CACCUGCAAUGUCUGGAAGAGGAACUGAAGCCCCUGGAAGAGGUGCUUAAUCUGGCUCAGAGCAAGA | |
| | ACUUCCACCUGAGACCUCGGGACCUGAUCAGCGACAUCAACGUGAUCGUGCUGGAACUGAAAGGCAG | |
| | CGAGACAACCUUCAUGUGCGAGUACGCCGAUGAGACCGCCACCAUCGUGGAGUUCCUGAAUCGGUGG | |
| | AUCACCUUCUGCCAGUCCAUCAUCUCCACCCUGACCUGAUAAUAGAGACGCUACGGACUUAAAUAAU | |
| | UGAGCCUUAAAGAAGAAAUUCUUUAAGUGGAUGCUCUCAAACUCAGGGAAACCUAAAUCUAGUUAUA | |
| | GACAAGGCAAUCCUGAGCCAAGCCGAAGUAGUAAUUAGUAAGACCAGUGGACAAUCGACGGAUAACA | |
| | GCAUAUCUAG | |
| 528 | GGGAGACCCUCGACCGUCGAUUGUCCACUGGUCAACAAUAGAUGACUUACAACUAAUCGGAAGGUGC | AnaX-CLEAN- |
| | AGAGACUCGACGGGAGCUACCCUAACGUCAAGACGAGGGUAAAGAGAGAGUCCAAUUCUCAAAGCCA | PIE-IRES- |
| | AUAGGCAGUAGCGAAAGCUGCAAGAGAAUGAGAAGUAACACACACCGAUCAACAGUCAGCGUGGCAC | rawCVB3- |
| | ACCAGCCACGUUUUGAUCAAGCACUUCUGUUACCCCGGACUGAGUAUCAAUAGACUGCUCACGCGGU | CVB3-IL2 |
| | UGAAGGAGAAAGCGUUCGUUAUCCGGCCAACUACUUCGAAAAACCUAGUAACACCGUGGAAGUUGCA | precursor |
| | GAGUGUUUCGCUCAGCACUACCCCAGUGUAGAUCAGGUCGAUGAGUCACCGCAUUCCCCACGGGCGA | |
| | CCGUGGCGGUGGCUGCGUUGGCGGCCUGCCCAUGGGGAAACCCAUGGGACGCUCUAAUACAGACAUG | |
| | GUGCGAAGAGUCUAUUGAGCUAGUUGGUAGUCCUCCGGCCCCUGAAUGCGGCUAAUCCUAACUGCGG | |
| | AGCACACACCCUCAAGCCAGAGGGCAGUGUGUCGUAACGGGCAACUCUGCAGCGGAACCGACUACUU | |
| | UGGGUGUCCGUGUUUCAUUUUAUUCCUAUACUGGCUGCUUAUGGUGACAAUUGAGAGAUCGUUACCA | |
| | UAUAGCUAUUGGAUUGGCCAUCCGGUGACUAAUAGAGCUAUUAUAUAUCCCUUUGUUGGGUUUAUAC | |
| | CACUUAGCUUGAAAGAGGUUAAAACAUUACAAUUCAUUGUUAAGUUGAAUACAGCAAAGCCACCAUG | |
| | UAUAGAAUGCAGCUGCUGUCUUGCAUCGCCCUGAGCCUGGCCCUGGUCACAAACAGCGCUCCUACAA | |
| | GCUCUAGCACCAAGAAAACCCAGCUGCAGCUCGAGCACCUGCUGCUGGACCUGCAGAUGAUCCUGAA | |
| | CGGCAUUAACAACUACAAGAACCCAAAGCUGACCAGAAUGCUGACAUUUAAGUUCUACAUGCCUAAG | |
| | AAGGCCACAGAGCUGAAGCACCUGCAAUGUCUGGAAGAGGAACUGAAGCCCCUGGAAGAGGUGCUUA | |
| | AUCUGGCUCAGAGCAAGAACUUCCACCUGAGACCUCGGGACCUGAUCAGCGACAUCAACGUGAUCGU | |
| | GCUGGAACUGAAAGGCAGCGAGACAACCUUCAUGUGCGAGUACGCCGAUGAGACCGCCACCAUCGUG | |
| | GAGUUCCUGAAUCGGUGGAUCACCUUCUGCCAGUCCAUCAUCUCCACCCUGACCUGAUAAUUAAAAC | |
| | AGCCUGUGGGUUGAUCCCACCCACAGGCCCAUUGGGCGCUAGCACUCUGGUAUCACGGUACCUUUGU | |
| | GCGCCUGUUUUAUACCCCCUCCCCCAACUGUAACUUAAAUAAUUGAGCCUUAAAGAAGAAAUUCUUU | |
| | AAGUGGAUGCUCUCAAACUCAGGGAAACCUAAAUCUAGUUAUAGACAAGGCAAUCCUGAGCCAAGCC | |
| | GAAGUAGUAAUUAGUAAGACCAGUGGACAAUCGACGGAUAACAGCAUAUCUAG | |
| 529 | GGGAGACCCUCGACCGUCGAUUGUCCACUGGUCAACAAUAGAUGACUUACAACUAAUCGGAAGGUGC | AnaX-CLEAN- |
| | AGAGACUCGACGGGAGCUACCCUAACGUCAAGACGAGGGUAAAGAGAGAGUCCAAUUCUCAAAGCCA | PIE-IRES- |
| | AUAGGCAGUAGCGAAAGCUGCAAGAGAAUGAAAAUUACAACACACCGAUCAACAGUCAGCGUGGCAC | rawMutation2- |
| | ACCAGCCACGUUUUGAUCAAGCACUUCUGUUACCCCGGACUGAGUAUCAAUAGACUGCUCACGCGGU | CVB3-IL2 |
| | UGAAGGAGAAAGCGUUCGUUAUCCGGCCAACUACUUCGAAAAACCUAGUAACACCGUGGAAGUUGCA | precursor |
| | GAGUGUUUCGCUCAGCACUACCCCAGUGUAGAUCAGGUCGAUGAGUCACCGCAUUCCCCACGGGCGA | |
| | CCGUGGCGGUGGCUGCGUUGGCGGCCUGCCCAUGGGGAAACCCAUGGGACGCUCUAAUACAGACAUG | |
| | GUGCGAAGAGUCUAUUGAGCUAGUUGGUAGUCCUCCGGCCCCUGAAUGCGGCUAAUCCUAACUGCGG | |
| | AGCACACACCCUCAAGCCAGAGGGCAGUGUGUCGUAACGGGCAACUCUGCAGCGGAACCGACUACUU | |
| | UGGGUGUCCGUGUUUCAUUUUAUUCCUAUACUGGCUGCUUAUGGUGACAAUUGAGAGAUCGUUACCA | |
| | UAUAGCUAUUGGAUUGGCCAUCCGGUGACUAAUAGAGCUAUUAUAUAUCCCUUUGUUGGGUUUAUAC | |
| | CACUUAGCUUGAAAGAGGUUAAAACAUUACAAUUCAUUGUUAAGUUGAAUACAGCAAAGCCACCAUG | |
| | UAUAGAAUGCAGCUGCUGUCUUGCAUCGCCCUGAGCCUGGCCCUGGUCACAAACAGCGCUCCUACAA | |
| | GCUCUAGCACCAAGAAAACCCAGCUGCAGCUCGAGCACCUGCUGCUGGACCUGCAGAUGAUCCUGAA | |
| | CGGCAUUAACAACUACAAGAACCCAAAGCUGACCAGAAUGCUGACAUUUAAGUUCUACAUGCCUAAG | |
| | AAGGCCACAGAGCUGAAGCACCUGCAAUGUCUGGAAGAGGAACUGAAGCCCCUGGAAGAGGUGCUUA | |
| | AUCUGGCUCAGAGCAAGAACUUCCACCUGAGACCUCGGGACCUGAUCAGCGACAUCAACGUGAUCGU | |
| | GCUGGAACUGAAAGGCAGCGAGACAACCUUCAUGUGCGAGUACGCCGAUGAGACCGCCACCAUCGUG | |
| | GAGUUCCUGAAUCGGUGGAUCACCUUCUGCCAGUCCAUCAUCUCCACCCUGACCUGAUAAUUAAAAC | |
| | AGCCUGUGGGUUGAUCCCACCCACAGGCCCAUUGGGCGCUAGCACUCUGGUAUCACGGUACCUUUGU | |
| | GCGCCUGUUUUAUACCCCCUCCCCCAACUGUAACUUAAAUAAUUGAGCCUUAAAGAAGAAAUUCUUU | |
| | AAGUGGAUGCUCUCAAACUCAGGGAAACCUAAAUCUAGUUAUAGACAAGGCAAUCCUGAGCCAAGCC | |
| | GAAGUAGUAAUUAGUAAGACCAGUGGACAAUCGACGGAUAACAGCAUAUCUAG | |
| 530 | GGGAGACCCUCGACCGUCGAUUGUCCACUGGUCAACAAUAGAUGACUUACAACUAAUCGGAAGGUGC | AnaX-CLEAN- |
| | AGAGACUCGACGGGAGCUACCCUAACGUCAAGACGAGGGUAAAGAGAGAGUCCAAUUCUCAAAGCCA | PIE-Cargo- |
| | AUAGGCAGUAGCGAAAGCUGCAAGAGAAUGAAACCUCGCUCAGAGCAAGAACUUCCACCUGAGACCU | PIE1-CVB3- |
| | CGGGACCUGAUCAGCGACAUCAACGUGAUCGUGCUGGAACUGAAAGGCAGCGAGACAACCUUCAUGU | IL2 |
| | GCGAGUACGCCGAUGAGACCGCCACCAUCGUGGAGUUCCUGAAUCGGUGGAUCACCUUCUGCCAGUC | precursor |
| | CAUCAUCUCCACCCUGACCUGAUAAUUAAAACAGCCUGUGGGUUGAUCCCACCCACAGGCCCAUUGG | |
| | GCGCUAGCACUCUGGUAUCACGGUACCUUUGUGCGCCUGUUUUAUACCCCCUCCCCCAACUGUAACU | |
| | UAGAAGUAACACACACCGAUCAACAGUCAGCGUGGCACACCAGCCACGUUUUGAUCAAGCACUUCUG | |
| | UUACCCCGGACUGAGUAUCAAUAGACUGCUCACGCGGUUGAAGGAGAAAGCGUUCGUUAUCCGGCCA | |
| | ACUACUUCGAAAAACCUAGUAACACCGUGGAAGUUGCAGAGUGUUUCGCUCAGCACUACCCCAGUGU | |
| | AGAUCAGGUCGAUGAGUCACCGCAUUCCCCACGGGCGACCGUGGCGGUGGCUGCGUUGGCGGCCUGC | |
| | CCAUGGGGAAACCCAUGGGACGCUCUAAUACAGACAUGGUGCGAAGAGUCUAUUGAGCUAGUUGGUA | |
| | GUCCUCCGGCCCCUGAAUGCGGCUAAUCCUAACUGCGGAGCACACACCCUCAAGCCAGAGGGCAGUG | |
| | UGUCGUAACGGGCAACUCUGCAGCGGAACCGACUACUUUGGGUGUCCGUGUUUCAUUUUAUUCCUAU | |
| | ACUGGCUGCUUAUGGUGACAAUUGAGAGAUCGUUACCAUAUAGCUAUUGGAUUGGCCAUCCGGUGAC | |
| | UAAUAGAGCUAUUAUAUAUCCCUUUGUUGGGUUUAUACCACUUAGCUUGAAAGAGGUUAAAACAUUA | |
| | CAAUUCAUUGUUAAGUUGAAUACAGCAAAGCCACCAUGUAUAGAAUGCAGCUGCUGUCUUGCAUCGC | |
| | CCUGAGCCUGGCCCUGGUCACAAACAGCGCUCCUACAAGCUCUAGCACCAAGAAAACCCAGCUGCAG | |
| | CUCGAGCACCUGCUGCUGGACCUGCAGAUGAUCCUGAACGGCAUUAACAACUACAAGAACCCAAAGC | |
| | UGACCAGAAUGCUGACAUUUAAGUUCUACAUGCCUAAGAAGGCCACAGAGCUGAAGCACCUGCAAUG | |
| | UCUGGAAGAGGAACUGAAGCCCCUGGAAGAGGUCUUAAAUAAUUGAGCCUUAAAGAAGAAAUUCUUU | |
| | AAGUGGAUGCUCUCAAACUCAGGGAAACCUAAAUCUAGUUAUAGACAAGGCAAUCCUGAGCCAAGCC | |
| | GAAGUAGUAAUUAGUAAGACCAGUGGACAAUCGACGGAUAACAGCAUAUCUAG | |
| 531 | GGGAGACCCUCGACCGUCGAUUGUCCACUGGUCAACAAUAGAUGACUUACAACUAAUCGGAAGGUGC | AnaX-CLEAN- |
| | AGAGACUCGACGGGAGCUACCCUAACGUCAAGACGAGGGUAAAGAGAGAGUCCAAUUCUCAAAGCCA | PIE-Cargo- |
| | AUAGGCAGUAGCGAAAGCUGCAAGAGAAUGAAUCUCUGACAUCAACGUGAUCGUGCUGGAACUGAAA | PIE2-CVB3- |
| | GGCAGCGAGACAACCUUCAUGUGCGAGUACGCCGAUGAGACCGCCACCAUCGUGGAGUUCCUGAAUC | IL2 |
| | GGUGGAUCACCUUCUGCCAGUCCAUCAUCUCCACCCUGACCUGAUAAUUAAAACAGCCUGUGGGUUG | precursor |
| | AUCCCACCCACAGGCCCAUUGGGCGCUAGCACUCUGGUAUCACGGUACCUUUGUGCGCCUGUUUUAU | |
| | ACCCCCUCCCCCAACUGUAACUUAGAAGUAACACACACCGAUCAACAGUCAGCGUGGCACACCAGCC | |
| | ACGUUUUGAUCAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUUGCAGAGUGUU | |
| | UCGCUCAGCACUACCCCAGUGUAGAUCAGGUCGAUGAGUCACCGCAUUCCCCACGGGCGACCGUGGC | |
| | GGUGGCUGCGUUGGCGGCCUGCCCAUGGGGAAACCCAUGGGACGCUCUAAUACAGACAUGGUGCGAA | |
| | GAGUCUAUUGAGCUAGUUGGUAGUCCUCCGGCCCCUGAAUGCGGCUAAUCCUAACUGCGGAGCACAC | |
| | ACCCUCAAGCCAGAGGGCAGUGUGUCGUAACGGGCAACUCUGCAGCGGAACCGACUACUUUGGGUGU | |
| | CCGUGUUUCAUUUUAUUCCUAUACUGGCUGCUUAUGGUGACAAUUGAGAGAUCGUUACCAUAUAGCU | |
| | AUUGGAUUGGCCAUCCGGUGACUAAUAGAGCUAUUAUAUAUCCCUUUGUUGGGUUUAUACCACUUAG | |
| | CUUGAAAGAGGUUAAAACAUUACAAUUCAUUGUUAAGUUGAAUACAGCAAAGCCACCAUGUAUAGAA | |
| | UGCAGCUGCUGUCUUGCAUCGCCCUGAGCCUGGCCCUGGUCACAAACAGCGCUCCUACAAGCUCUAG | |
| | CACCAAGAAAACCCAGCUGCAGCUCGAGCACCUGCUGCUGGACCUGCAGAUGAUCCUGAACGGCAUU | |
| | AACAACUACAAGAACCCAAAGCUGACCAGAAUGCUGACAUUUAAGUUCUACAUGCCUAAGAAGGCCA | |
| | CAGAGCUGAAGCACCUGCAAUGUCUGGAAGAGGAACUGAAGCCCCUGGAAGAGGUGCUUAAUCUGGC | |
| | UCAGAGCAAGAACUUCCACCUGAGACCUAGAGACUUAAAUAAUUGAGCCUUAAAGAAGAAAUUCUUU | |
| | AAGUGGAUGCUCUCAAACUCAGGGAAACCUAAAUCUAGUUAUAGACAAGGCAAUCCUGAGCCAAGCC | |
| | GAAGUAGUAAUUAGUAAGACCAGUGGACAAUCGACGGAUAACAGCAUAUCUAG | |
| 532 | GGGAGACCCUCGACCGUCGAUUGUCCACUGGUCCCGUCGAUUGUCCACUGGUCAACAAUAGAUGACU | AnaX-GCv3- |
| | UACAACUAAUCGGAAGGUGCAGAGACUCGACGGGAGCUACCCUAACGUCAAGACGAGGGUAAAGAGA | 2 x homo-CVB3- |
| | GAGUCCAAUUCUCAAAGCCAAUAGGCAGUAGCGAAAGCUGCAAGAGAAUGAAAACCCCCCCAUUAAA | AQP1 |
| | ACAGCCUGUGGGUUGAUCCCACCCACAGGCCCAUUGGGCGCUAGCACUCUGGUAUCACGGUACCUUU | precursor |
| | GUGCGCCUGUUUUAUACCCCCUCCCCCAACUGUAACUUAGAAGUAACACACACCGAUCAACAGUCAG | |
| | CGUGGCACACCAGCCACGUUUUGAUCAAGCACUUCUGUUACCCCGGACUGAGUAUCAAUAGACUGCU | |
| | CACGCGGUUGAAGGAGAAAGCGUUCGUUAUCCGGCCAACUACUUCGAAAAACCUAGUAACACCGUGG | |
| | AAGUUGCAGAGUGUUUCGCUCAGCACUACCCCAGUGUAGAUCAGGUCGAUGAGUCACCGCAUUCCCC | |
| | ACGGGCGACCGUGGCGGUGGCUGCGUUGGCGGCCUGCCCAUGGGGAAACCCAUGGGACGCUCUAAUA | |
| | CAGACAUGGUGCGAAGAGUCUAUUGAGCUAGUUGGUAGUCCUCCGGCCCCUGAAUGCGGCUAAUCCU | |
| | AACUGCGGAGCACACACCCUCAAGCCAGAGGGCAGUGUGUCGUAACGGGCAACUCUGCAGCGGAACC | |
| | GACUACUUUGGGUGUCCGUGUUUCAUUUUAUUCCUAUACUGGCUGCUUAUGGUGACAAUUGAGAGAU | |
| | CGUUACCAUAUAGCUAUUGGAUUGGCCAUCCGGUGACUAAUAGAGCUAUUAUAUAUCCCUUUGUUGG | |
| | GUUUAUACCACUUAGCUUGAAAGAGGUUAAAACAUUACAAUUCAUUGUUAAGUUGAAUACAGCAAAG | |
| | CCACCAUGGCCAGCGAGUUCAAGAAGAAGCUCUUCUGGAGGGCAGUGGUGGCCGAGUUCCUGGCCAC | |
| | GACCCUCUUUGUCUUCAUCAGCAUCGGUUCUGCCCUGGGCUUCAAAUACCCGGUGGGGAACAACCAG | |
| | ACGGCGGUCCAGGACAACGUGAAGGUGUCGCUGGCCUUCGGGCUGAGCAUCGCCACGCUGGCGCAGA | |
| | GUGUGGGCCACAUCAGCGGCGCCCACCUCAACCCGGCUGUCACACUGGGGCUGCUGCUCAGCUGCCA | |
| | GAUCAGCAUCUUCCGUGCCCUCAUGUACAUCAUCGCCCAGUGCGUGGGGGCCAUCGUCGCCACCGCC | |
| | AUCCUCUCAGGCAUCACCUCCUCCCUGACUGGGAACUCGCUUGGCCGCAAUGACCUGGCUGAUGGUG | |
| | UGAACUCGGGCCAGGGCCUGGGCAUCGAGAUCAUCGGGACCCUCCAGCUGGUGCUAUGCGUGCUGGC | |
| | UACUACCGACCGGAGGCGCCGUGACCUUGGUGGCUCAGCCCCCCUUGCCAUCGGCCUCUCUGUAGCC | |
| | CUUGGACACCUCCUGGCUAUUGACUACACUGGCUGUGGGAUUAACCCUGCUCGGUCCUUUGGCUCCG | |
| | CGGUGAUCACACACAACUUCAGCAACCACUGGAUUUUCUGGGUGGGGCCAUUCAUCGGGGGAGCCCU | |
| | GGCUGUACUCAUCUACGACUUCAUCCUGGCCCCACGCAGCAGUGACCUCACAGACCGCGUGAAGGUG | |
| | UGGACCAGCGGCCAGGUGGAGGAGUAUGACCUGGAUGCCGACGACAUCAACUCCAGGGUGGAGAUGA | |
| | AGCCCAAAUGAUAAAGACGGGGGGGGCUUAAAUAAUUGAGCCUUAAAGAAGAAAUUCUUUAAGUGGA | |
| | UGCUCUCAAACUCAGGGAAACCUAAAUCUAGUUAUAGACAAGGCAAUCCUGAGCCAAGCCGAAGUAG | |
| | UAAUUAGUAAGACCAGUGGACAAUCGACGGGACCAGUGGACAAUCGACGGAUAACAGCAUAUCUAG | |
| 533 | GGGAGACCCUCGACCGUCGAUUGUCCACUGGUCCCGUCGAUUGUCCACUGGUCAACAAUAGAUGACU | AnaX-GCv3- |
| | UACAACUAAUCGGAAGGUGCAGAGACUCGACGGGAGCUACCCUAACGUCAAGACGAGGGUAAAGAGA | 2 x homo- |
| | GAGUCCAAUUCUCAAAGCCAAUAGGCAGUAGCGAAAGCUGCAAGAGAAUGAACUGGAAACUCGCUUG | CLEAN-PIE- |
| | GCCGCAAUGACCUGGCUGAUGGUGUGAACUCGGGCCAGGGCCUGGGCAUCGAGAUCAUCGGGACCCU | Cargo-PIE1- |
| | CCAGCUGGUGCUAUGCGUGCUGGCUACUACCGACCGGAGGCGCCGUGACCUUGGUGGCUCAGCCCCC | CVB3-AQP1 |
| | CUUGCCAUCGGCCUCUCUGUAGCCCUUGGACACCUCCUGGCUAUUGACUACACUGGCUGUGGGAUUA | precursor |
| | ACCCUGCUCGGUCCUUUGGCUCCGCGGUGAUCACACACAACUUCAGCAACCACUGGAUUUUCUGGGU | |
| | GGGGCCAUUCAUCGGGGGAGCCCUGGCUGUACUCAUCUACGACUUCAUCCUGGCCCCACGCAGCAGU | |
| | GACCUCACAGACCGCGUGAAGGUGUGGACCAGCGGCCAGGUGGAGGAGUAUGACCUGGAUGCCGACG | |
| | ACAUCAACUCCAGGGUGGAGAUGAAGCCCAAAUGAUAAUUAAAACAGCCUGUGGGUUGAUCCCACCC | |
| | ACAGGCCCAUUGGGCGCUAGCACUCUGGUAUCACGGUACCUUUGUGCGCCUGUUUUAUACCCCCUCC | |
| | CCCAACUGUAACUUAGAAGUAACACACACCGAUCAACAGUCAGCGUGGCACACCAGCCACGUUUUGA | |
| | UCAAGCACUUCUGUUACCCCGGACUGAGUAUCAAUAGACUGCUCACGCGGUUGAAGGAGAAAGCGUU | |
| | CGUUAUCCGGCCAACUACUUCGAAAAACCUAGUAACACCGUGGAAGUUGCAGAGUGUUUCGCUCAGC | |
| | ACUACCCCAGUGUAGAUCAGGUCGAUGAGUCACCGCAUUCCCCACGGGCGACCGUGGCGGUGGCUGC | |
| | GUUGGCGGCCUGCCCAUGGGGAAACCCAUGGGACGCUCUAAUACAGACAUGGUGCGAAGAGUCUAUU | |
| | GAGCUAGUUGGUAGUCCUCCGGCCCCUGAAUGCGGCUAAUCCUAACUGCGGAGCACACACCCUCAAG | |
| | CCAGAGGGCAGUGUGUCGUAACGGGCAACUCUGCAGCGGAACCGACUACUUUGGGUGUCCGUGUUUC | |
| | AUUUUAUUCCUAUACUGGCUGCUUAUGGUGACAAUUGAGAGAUCGUUACCAUAUAGCUAUUGGAUUG | |
| | GCCAUCCGGUGACUAAUAGAGCUAUUAUAUAUCCCUUUGUUGGGUUUAUACCACUUAGCUUGAAAGA | |
| | GGUUAAAACAUUACAAUUCAUUGUUAAGUUGAAUACAGCAAAGCCACCAUGGCCAGCGAGUUCAAGA | |
| | AGAAGCUCUUCUGGAGGGCAGUGGUGGCCGAGUUCCUGGCCACGACCCUCUUUGUCUUCAUCAGCAU | |
| | CGGUUCUGCCCUGGGCUUCAAAUACCCGGUGGGGAACAACCAGACGGCGGUCCAGGACAACGUGAAG | |
| | GUGUCGCUGGCCUUCGGGCUGAGCAUCGCCACGCUGGCGCAGAGUGUGGGCCACAUCAGCGGCGCCC | |
| | ACCUCAACCCGGCUGUCACACUGGGGCUGCUGCUCAGCUGCCAGAUCAGCAUCUUCCGUGCCCUCAU | |
| | GUACAUCAUCGCCCAGUGCGUGGGGGCCAUCGUCGCCACCGCCAUCCUCUCAGGCAUCACCUCCAGC | |
| | UUAAAUAAUUGAGCCUUAAAGAAGAAAUUCUUUAAGUGGAUGCUCUCAAACUCAGGGAAACCUAAAU | |
| | CUAGUUAUAGACAAGGCAAUCCUGAGCCAAGCCGAAGUAGUAAUUAGUAAGACCAGUGGACAAUCGA | |
| | CGGGACCAGUGGACAAUCGACGGAUAACAGCAUAUCUAG | |
| 534 | GGGAGACCCUCGACCGUCGAUUGUCCACUGGUCAAUAAGAGAAAGUGCAACGACUAUUCCGAUAGGA | Twort-PIE-6- |
| | AGUAGGGUCAAGUGACUCGAAAUGGGGAUUACCCUUCUAGGGUAGUGAUAUAGUCUGAACAUAUAUG | CLEAN-PIE- |
| | GAAACAUAUAGAAGGAUAGGAGUAACGAACCUAUUCGUAACAUAAUUGAACCAGCUCCCUGACUGGG | Cargo-PIE1- |
| | AACUCGCUUGGCCGCAAUGACCUGGCUGAUGGUGUGAACUCGGGCCAGGGCCUGGGCAUCGAGAUCA | CVB3-AQP1 |
| | UCGGGACCCUCCAGCUGGUGCUAUGCGUGCUGGCUACUACCGACCGGAGGCGCCGUGACCUUGGUGG | precursor |
| | CUCAGCCCCCCUUGCCAUCGGCCUCUCUGUAGCCCUUGGACACCUCCUGGCUAUUGACUACACUGGC | |
| | UGUGGGAUUAACCCUGCUCGGUCCUUUGGCUCCGCGGUGAUCACACACAACUUCAGCAACCACUGGA | |
| | UUUUCUGGGUGGGGCCAUUCAUCGGGGGAGCCCUGGCUGUACUCAUCUACGACUUCAUCCUGGCCCC | |
| | ACGCAGCAGUGACCUCACAGACCGCGUGAAGGUGUGGACCAGCGGCCAGGUGGAGGAGUAUGACCUG | |
| | GAUGCCGACGACAUCAACUCCAGGGUGGAGAUGAAGCCCAAAUGAUAAUUAAAACAGCCUGUGGGUU | |
| | GAUCCCACCCACAGGCCCAUUGGGCGCUAGCACUCUGGUAUCACGGUACCUUUGUGCGCCUGUUUUA | |
| | UACCCCCUCCCCCAACUGUAACUUAGAAGUAACACACACCGAUCAACAGUCAGCGUGGCACACCAGC | |
| | CACGUUUUGAUCAAGCACUUCUGUUACCCCGGACUGAGUAUCAAUAGACUGCUCACGCGGUUGAAGG | |
| | AGAAAGCGUUCGUUAUCCGGCCAACUACUUCGAAAAACCUAGUAACACCGUGGAAGUUGCAGAGUGU | |
| | UUCGCUCAGCACUACCCCAGUGUAGAUCAGGUCGAUGAGUCACCGCAUUCCCCACGGGCGACCGUGG | |
| | CGGUGGCUGCGUUGGCGGCCUGCCCAUGGGGAAACCCAUGGGACGCUCUAAUACAGACAUGGUGCGA | |
| | AGAGUCUAUUGAGCUAGUUGGUAGUCCUCCGGCCCCUGAAUGCGGCUAAUCCUAACUGCGGAGCACA | |
| | CACCCUCAAGCCAGAGGGCAGUGUGUCGUAACGGGCAACUCUGCAGCGGAACCGACUACUUUGGGUG | |
| | UCCGUGUUUCAUUUUAUUCCUAUACUGGCUGCUUAUGGUGACAAUUGAGAGAUCGUUACCAUAUAGC | |
| | UAUUGGAUUGGCCAUCCGGUGACUAAUAGAGCUAUUAUAUAUCCCUUUGUUGGGUUUAUACCACUUA | |
| | GCUUGAAAGAGGUUAAAACAUUACAAUUCAUUGUUAAGUUGAAUACAGCAAAGCCACCAUGGCCAGC | |
| | GAGUUCAAGAAGAAGCUCUUCUGGAGGGCAGUGGUGGCCGAGUUCCUGGCCACGACCCUCUUUGUCU | |
| | UCAUCAGCAUCGGUUCUGCCCUGGGCUUCAAAUACCCGGUGGGGAACAACCAGACGGCGGUCCAGGA | |
| | CAACGUGAAGGUGUCGCUGGCCUUCGGGCUGAGCAUCGCCACGCUGGCGCAGAGUGUGGGCCACAUC | |
| | AGCGGCGCCCACCUCAACCCGGCUGUCACACUGGGGCUGCUGCUCAGCUGCCAGAUCAGCAUCUUCC | |
| | GUGCCCUCAUGUACAUCAUCGCCCAGUGCGUGGGGGCCAUCGUCGCCACCGCCAUCCUCUCUGGCAU | |
| | AAAUAAUUGUGCCUUUAUACAGUAAUGUAUAUCGAAAAAUCCUCUAAUUCAGGGAACACCUAAACAA | |
| | ACUAAGAUGUAGGCAAUCCUGAGCUAAGCUCUUAGUGACCAGUGGACAAUCGACGGAUAACAGCAUA | |
| | UCUAG | |
| 535 | GGGAGACCCUCGACCGUCGAUUGUCCACUGGUCCCGUCGAUUGUCCACUGGUCAACAAUAGAUGACU | AnaX-GCv3- |
| | UACAACUAAUCGGAAGGUGCAGAGACUCGACGGGAGCUACCCUAACGUCAAGACGAGGGUAAAGAGA | 2 × homo- |
| | GAGUCCAAUUCUCAAAGCCAAUAGGCAGUAGCGAAAGCUGCAAGAGAAUGAGAAGUAACACACACCG | CLEAN-PIE- |
| | AUCAACAGUCAGCGUGGCACACCAGCCACGUUUUGAUCAAGCACUUCUGUUACCCCGGACUGAGUAU | IRES- |
| | CAAUAGACUGCUCACGCGGUUGAAGGAGAAAGCGUUCGUUAUCCGGCCAACUACUUCGAAAAACCUA | rawCVB3-AQP1 |
| | GUAACACCGUGGAAGUUGCAGAGUGUUUCGCUCAGCACUACCCCAGUGUAGAUCAGGUCGAUGAGUC | precursor |
| | ACCGCAUUCCCCACGGGCGACCGUGGCGGUGGCUGCGUUGGCGGCCUGCCCAUGGGGAAACCCAUGG | |
| | GACGCUCUAAUACAGACAUGGUGCGAAGAGUCUAUUGAGCUAGUUGGUAGUCCUCCGGCCCCUGAAU | |
| | GCGGCUAAUCCUAACUGCGGAGCACACACCCUCAAGCCAGAGGGCAGUGUGUCGUAACGGGCAACUC | |
| | UGCAGCGGAACCGACUACUUUGGGUGUCCGUGUUUCAUUUUAUUCCUAUACUGGCUGCUUAUGGUGA | |
| | CAAUUGAGAGAUCGUUACCAUAUAGCUAUUGGAUUGGCCAUCCGGUGACUAAUAGAGCUAUUAUAUA | |
| | UCCCUUUGUUGGGUUUAUACCACUUAGCUUGAAAGAGGUUAAAACAUUACAAUUCAUUGUUAAGUUG | |
| | AAUACAGCAAAGCCACCAUGGCCAGCGAGUUCAAGAAGAAGCUCUUCUGGAGGGCAGUGGUGGCCGA | |
| | GUUCCUGGCCACGACCCUCUUUGUCUUCAUCAGCAUCGGUUCUGCCCUGGGCUUCAAAUACCCGGUG | |
| | GGGAACAACCAGACGGCGGUCCAGGACAACGUGAAGGUGUCGCUGGCCUUCGGGCUGAGCAUCGCCA | |
| | CGCUGGCGCAGAGUGUGGGCCACAUCAGCGGCGCCCACCUCAACCCGGCUGUCACACUGGGGCUGCU | |
| | GCUCAGCUGCCAGAUCAGCAUCUUCCGUGCCCUCAUGUACAUCAUCGCCCAGUGCGUGGGGGCCAUC | |
| | GUCGCCACCGCCAUCCUCUCAGGCAUCACCUCCUCCCUGACUGGGAACUCGCUUGGCCGCAAUGACC | |
| | UGGCUGAUGGUGUGAACUCGGGCCAGGGCCUGGGCAUCGAGAUCAUCGGGACCCUCCAGCUGGUGCU | |
| | AUGCGUGCUGGCUACUACCGACCGGAGGCGCCGUGACCUUGGUGGCUCAGCCCCCCUUGCCAUCGGC | |
| | CUCUCUGUAGCCCUUGGACACCUCCUGGCUAUUGACUACACUGGCUGUGGGAUUAACCCUGCUCGGU | |
| | CCUUUGGCUCCGCGGUGAUCACACACAACUUCAGCAACCACUGGAUUUUCUGGGUGGGGCCAUUCAU | |
| | CGGGGGAGCCCUGGCUGUACUCAUCUACGACUUCAUCCUGGCCCCACGCAGCAGUGACCUCACAGAC | |
| | CGCGUGAAGGUGUGGACCAGCGGCCAGGUGGAGGAGUAUGACCUGGAUGCCGACGACAUCAACUCCA | |
| | GGGUGGAGAUGAAGCCCAAAUGAUAAUUAAAACAGCCUGUGGGUUGAUCCCACCCACAGGCCCAUUG | |
| | GGCGCUAGCACUCUGGUAUCACGGUACCUUUGUGCGCCUGUUUUAUACCCCCUCCCCCAACUGUAAC | |
| | UUAAAUAAUUGAGCCUUAAAGAAGAAAUUCUUUAAGUGGAUGCUCUCAAACUCAGGGAAACCUAAAU | |
| | CUAGUUAUAGACAAGGCAAUCCUGAGCCAAGCCGAAGUAGUAAUUAGUAAGACCAGUGGACAAUCGA | |
| | CGGGACCAGUGGACAAUCGACGGAUAACAGCAUAUCUAG | |
| 536 | GGGAGACCCUCGACCGUCGAUUGUCCACUGGUCCCGUCGAUUGUCCACUGGUCAACAAUAGAUGACU | AnaX-GCv3- |
| | UACAACUAAUCGGAAGGUGCAGAGACUCGACGGGAGCUACCCUAACGUCAAGACGAGGGUAAAGAGA | 2 × homo- |
| | GAGUCCAAUUCUCAAAGCCAAUAGGCAGUAGCGAAAGCUGCAAGAGAAUGAAAAUUACAACACACCG | CLEAN-PIE- |
| | AUCAACAGUCAGCGUGGCACACCAGCCACGUUUUGAUCAAGCACUUCUGUUACCCCGGACUGAGUAU | IRES- |
| | CAAUAGACUGCUCACGCGGUUGAAGGAGAAAGCGUUCGUUAUCCGGCCAACUACUUCGAAAAACCUA | mutationCVB3- |
| | GUAACACCGUGGAAGUUGCAGAGUGUUUCGCUCAGCACUACCCCAGUGUAGAUCAGGUCGAUGAGUC | AQP1 |
| | ACCGCAUUCCCCACGGGCGACCGUGGCGGUGGCUGCGUUGGCGGCCUGCCCAUGGGGAAACCCAUGG | precursor |
| | GACGCUCUAAUACAGACAUGGUGCGAAGAGUCUAUUGAGCUAGUUGGUAGUCCUCCGGCCCCUGAAU | |
| | GCGGCUAAUCCUAACUGCGGAGCACACACCCUCAAGCCAGAGGGCAGUGUGUCGUAACGGGCAACUC | |
| | UGCAGCGGAACCGACUACUUUGGGUGUCCGUGUUUCAUUUUAUUCCUAUACUGGCUGCUUAUGGUGA | |
| | CAAUUGAGAGAUCGUUACCAUAUAGCUAUUGGAUUGGCCAUCCGGUGACUAAUAGAGCUAUUAUAUA | |
| | UCCCUUUGUUGGGUUUAUACCACUUAGCUUGAAAGAGGUUAAAACAUUACAAUUCAUUGUUAAGUUG | |
| | AAUACAGCAAAGCCACCAUGGCCAGCGAGUUCAAGAAGAAGCUCUUCUGGAGGGCAGUGGUGGCCGA | |
| | GUUCCUGGCCACGACCCUCUUUGUCUUCAUCAGCAUCGGUUCUGCCCUGGGCUUCAAAUACCCGGUG | |
| | GGGAACAACCAGACGGCGGUCCAGGACAACGUGAAGGUGUCGCUGGCCUUCGGGCUGAGCAUCGCCA | |
| | CGCUGGCGCAGAGUGUGGGCCACAUCAGCGGCGCCCACCUCAACCCGGCUGUCACACUGGGGCUGCU | |
| | GCUCAGCUGCCAGAUCAGCAUCUUCCGUGCCCUCAUGUACAUCAUCGCCCAGUGCGUGGGGGCCAUC | |
| | GUCGCCACCGCCAUCCUCUCAGGCAUCACCUCCUCCCUGACUGGGAACUCGCUUGGCCGCAAUGACC | |
| | UGGCUGAUGGUGUGAACUCGGGCCAGGGCCUGGGCAUCGAGAUCAUCGGGACCCUCCAGCUGGUGCU | |
| | AUGCGUGCUGGCUACUACCGACCGGAGGCGCCGUGACCUUGGUGGCUCAGCCCCCCUUGCCAUCGGC | |
| | CUCUCUGUAGCCCUUGGACACCUCCUGGCUAUUGACUACACUGGCUGUGGGAUUAACCCUGCUCGGU | |
| | CCUUUGGCUCCGCGGUGAUCACACACAACUUCAGCAACCACUGGAUUUUCUGGGUGGGGCCAUUCAU | |
| | CGGGGGAGCCCUGGCUGUACUCAUCUACGACUUCAUCCUGGCCCCACGCAGCAGUGACCUCACAGAC | |
| | CGCGUGAAGGUGUGGACCAGCGGCCAGGUGGAGGAGUAUGACCUGGAUGCCGACGACAUCAACUCCA | |
| | GGGUGGAGAUGAAGCCCAAAUGAUAAUUAAAACAGCCUGUGGGUUGAUCCCACCCACAGGCCCAUUG | |
| | GGCGCUAGCACUCUGGUAUCACGGUACCUUUGUGCGCCUGUUUUAUACCCCCUCCCCCAACUGUAAC | |
| | UUAAAUAAUUGAGCCUUAAAGAAGAAAUUCUUUAAGUGGAUGCUCUCAAACUCAGGGAAACCUAAAU | |
| | CUAGUUAUAGACAAGGCAAUCCUGAGCCAAGCCGAAGUAGUAAUUAGUAAGACCAGUGGACAAUCGA | |
| | CGGGACCAGUGGACAAUCGACGGAUAACAGCAUAUCUAG | |
| 537 | GGGAGACCCUCGAGGACACCCCCUGACCCGGUCUCAGAAAGCCUGAAAGCAGAAUUAGUCAUUAGAA | AnaX-MEHA1- |
| | GGGUGGUUGGCUUGGUCGGCAUAGACUUUGAGCAGAAAGAGGUUGAAAAUGUUGAGCCUGAUUUCUC | CVB3-IL2 |
| | UUAGGCCCCUCUGCAGUGUCUGUUGUGGAGGCCAGAUACGUAACUGCUUCCGCUUUUUUUGGUCUCA | precursor |
| | UUCAAGGUGAGCAAAUCCCCUUCAUGUUUCUCACCAGACAAUGCAGCUGAUGAGGUUCCAGCUUUGC | |
| | AAAUGUAGUCAUCCAUGAGGACUGUCUUCCUGAGAUUUCAUCAGGAACAAUAGAUGACUUACAACUA | |
| | AUCGGAAGGUGCAGAGACUCGACGGGAGCUACCCUAACGUCAAGACGAGGGUAAAGAGAGAGUCCAA | |
| | UUCUCAAAGCCAAUAGGCAGUAGCGAAAGCUGCAAGAGAAUGAAAACCCCCCCAUUAAAACAGCCUG | |
| | UGGGUUGAUCCCACCCACAGGCCCAUUGGGCGCUAGCACUCUGGUAUCACGGUACCUUUGUGCGCCU | |
| | GUUUUAUACCCCCUCCCCCAACUGUAACUUAGAAGUAACACACACCGAUCAACAGUCAGCGUGGCAC | |
| | ACCAGCCACGUUUUGAUCAAGCACUUCUGUUACCCCGGACUGAGUAUCAAUAGACUGCUCACGCGGU | |
| | UGAAGGAGAAAGCGUUCGUUAUCCGGCCAACUACUUCGAAAAACCUAGUAACACCGUGGAAGUUGCA | |
| | GAGUGUUUCGCUCAGCACUACCCCAGUGUAGAUCAGGUCGAUGAGUCACCGCAUUCCCCACGGGCGA | |
| | CCGUGGCGGUGGCUGCGUUGGCGGCCUGCCCAUGGGGAAACCCAUGGGACGCUCUAAUACAGACAUG | |
| | GUGCGAAGAGUCUAUUGAGCUAGUUGGUAGUCCUCCGGCCCCUGAAUGCGGCUAAUCCUAACUGCGG | |
| | AGCACACACCCUCAAGCCAGAGGGCAGUGUGUCGUAACGGGCAACUCUGCAGCGGAACCGACUACUU | |
| | UGGGUGUCCGUGUUUCAUUUUAUUCCUAUACUGGCUGCUUAUGGUGACAAUUGAGAGAUCGUUACCA | |
| | UAUAGCUAUUGGAUUGGCCAUCCGGUGACUAAUAGAGCUAUUAUAUAUCCCUUUGUUGGGUUUAUAC | |
| | CACUUAGCUUGAAAGAGGUUAAAACAUUACAAUUCAUUGUUAAGUUGAAUACAGCAAAGCCACCAUG | |
| | UAUAGAAUGCAGCUGCUGUCUUGCAUCGCCCUGAGCCUGGCCCUGGUCACAAACAGCGCUCCUACAA | |
| | GCUCUAGCACCAAGAAAACCCAGCUGCAGCUCGAGCACCUGCUGCUGGACCUGCAGAUGAUCCUGAA | |
| | CGGCAUUAACAACUACAAGAACCCAAAGCUGACCAGAAUGCUGACAUUUAAGUUCUACAUGCCUAAG | |
| | AAGGCCACAGAGCUGAAGCACCUGCAAUGUCUGGAAGAGGAACUGAAGCCCCUGGAAGAGGUGCUUA | |
| | AUCUGGCUCAGAGCAAGAACUUCCACCUGAGACCUCGGGACCUGAUCAGCGACAUCAACGUGAUCGU | |
| | GCUGGAACUGAAAGGCAGCGAGACAACCUUCAUGUGCGAGUACGCCGAUGAGACCGCCACCAUCGUG | |
| | GAGUUCCUGAAUCGGUGGAUCACCUUCUGCCAGUCCAUCAUCUCCACCCUGACCUGAUAAAGACGGG | |
| | GGGGGCUUAAAUAAUUGAGCCUUAAAGAAGAAAUUCUUUAAGUGGAUGCUCUCAAACUCAGGGAAAC | |
| | CUAAAUCUAGUUAUAGACAAGGCAAUCCUGAGCCAAGCCGAAGUAGUAAUUAGUAACCUGAUGAAAU | |
| | CUCAGGAAGACAGUCCUCAUGGAUGACUACAUUUGCAAAGCUGGAACCUCAUCAGCUGCAUUGUCUG | |
| | GUGAGAAACAUGAAGGGGAUUUGCUCACCUUGAAUGAGACCAAAAAAAGCGGAAGCAGUUACGUAUC | |
| | UGGCCUCCACAACAGACACUGCAGAGGGGCCUAAGAGAAAUCAGGCUCAACAUUUUCAACCUCUUUC | |
| | UGCUCAAAGUCUAUGCCGACCAAGCCAACCACCCUUCUAAUGACUAAUUCUGCUUUCAGGCUUUCUG | |
| | AGACCGGGUCAGGGGGUGUCCAUAACAGCAUAUCUAG | |
| 538 | GGGAGACCCUCGAGGACACCCCCUGACCCGGUCUCAGAAAGCCUGAAAGCAGAAUUAGUCAUUAGAA | AnaX-MEHA2- |
| | GGGUGGUUGGCUUGGUCGGCAUAGACUUUGAGCAGAAAGAGGUUGAAACAAUAGAUGACUUACAACU | CVB3-IL2 |
| | AAUCGGAAGGUGCAGAGACUCGACGGGAGCUACCCUAACGUCAAGACGAGGGUAAAGAGAGAGUCCA | precursor |
| | AUUCUCAAAGCCAAUAGGCAGUAGCGAAAGCUGCAAGAGAAUGAAAACCCCCCCAUUAAAACAGCCU | |
| | GUGGGUUGAUCCCACCCACAGGCCCAUUGGGCGCUAGCACUCUGGUAUCACGGUACCUUUGUGCGCC | |
| | UGUUUUAUACCCCCUCCCCCAACUGUAACUUAGAAGUAACACACACCGAUCAACAGUCAGCGUGGCA | |
| | CACCAGCCACGUUUUGAUCAAGCACUUCUGUUACCCCGGACUGAGUAUCAAUAGACUGCUCACGCGG | |
| | UUGAAGGAGAAAGCGUUCGUUAUCCGGCCAACUACUUCGAAAAACCUAGUAACACCGUGGAAGUUGC | |
| | AGAGUGUUUCGCUCAGCACUACCCCAGUGUAGAUCAGGUCGAUGAGUCACCGCAUUCCCCACGGGCG | |
| | ACCGUGGCGGUGGCUGCGUUGGCGGCCUGCCCAUGGGGAAACCCAUGGGACGCUCUAAUACAGACAU | |
| | GGUGCGAAGAGUCUAUUGAGCUAGUUGGUAGUCCUCCGGCCCCUGAAUGCGGCUAAUCCUAACUGCG | |
| | GAGCACACACCCUCAAGCCAGAGGGCAGUGUGUCGUAACGGGCAACUCUGCAGCGGAACCGACUACU | |
| | UUGGGUGUCCGUGUUUCAUUUUAUUCCUAUACUGGCUGCUUAUGGUGACAAUUGAGAGAUCGUUACC | |
| | AUAUAGCUAUUGGAUUGGCCAUCCGGUGACUAAUAGAGCUAUUAUAUAUCCCUUUGUUGGGUUUAUA | |
| | CCACUUAGCUUGAAAGAGGUUAAAACAUUACAAUUCAUUGUUAAGUUGAAUACAGCAAAGCCACCAU | |
| | GUAUAGAAUGCAGCUGCUGUCUUGCAUCGCCCUGAGCCUGGCCCUGGUCACAAACAGCGCUCCUACA | |
| | AGCUCUAGCACCAAGAAAACCCAGCUGCAGCUCGAGCACCUGCUGCUGGACCUGCAGAUGAUCCUGA | |
| | ACGGCAUUAACAACUACAAGAACCCAAAGCUGACCAGAAUGCUGACAUUUAAGUUCUACAUGCCUAA | |
| | GAAGGCCACAGAGCUGAAGCACCUGCAAUGUCUGGAAGAGGAACUGAAGCCCCUGGAAGAGGUGCUU | |
| | AAUCUGGCUCAGAGCAAGAACUUCCACCUGAGACCUCGGGACCUGAUCAGCGACAUCAACGUGAUCG | |
| | UGCUGGAACUGAAAGGCAGCGAGACAACCUUCAUGUGCGAGUACGCCGAUGAGACCGCCACCAUCGU | |
| | GGAGUUCCUGAAUCGGUGGAUCACCUUCUGCCAGUCCAUCAUCUCCACCCUGACCUGAUAAAGACGG | |
| | GGGGGGCUUAAAUAAUUGAGCCUUAAAGAAGAAAUUCUUUAAGUGGAUGCUCUCAAACUCAGGGAAA | |
| | CCUAAAUCUAGUUAUAGACAAGGCAAUCCUGAGCCAAGCCGAAGUAGUAAUUAGUAAUCAACCUCUU | |
| | UCUGCUCAAAGUCUAUGCCGACCAAGCCAACCACCCUUCUAAUGACUAAUUCUGCUUUCAGGCUUUC | |
| | UGAGACCGGGUCAGGGGGUGUCCAUAACAGCAUAUCUAG | |
| 539 | GGGAGACCCUCGAAAAGAAGACAGUCUCGCUCUAUCGCCCAGGCUGGAGUGUAGAAUGCAGUGCUGU | AnaX-MEHA3- |
| | GAUCUUGGCUCACUGCAACCUCUACCUCCCAGGCUCAAGCGAUUCUUCUGCCUCAGCCUCCCGAGUA | CVB3-IL2 |
| | GCUAGGAUUACAGGCGCGCGCCACCACACCUAGCUUAUUUUUGUAUUUUCAGUAGAGAUGGGGUUUC | precursor |
| | ACCAUGUUGACCAGGCUGGUCUCAAACUCCUGAGCUUAAGUGACGUGCCCGCCUCAGCCUCCCAAAG | |
| | UGCUUGGAUUAUAGGUGUGAGCCACUGCACAACAAUAGAUGACUUACAACUAAUCGGAAGGUGCAGA | |
| | GACUCGACGGGAGCUACCCUAACGUCAAGACGAGGGUAAAGAGAGAGUCCAAUUCUCAAAGCCAAUA | |
| | GGCAGUAGCGAAAGCUGCAAGAGAAUGAAAACCCCCCCAUUAAAACAGCCUGUGGGUUGAUCCCACC | |
| | CACAGGCCCAUUGGGCGCUAGCACUCUGGUAUCACGGUACCUUUGUGCGCCUGUUUUAUACCCCCUC | |
| | CCCCAACUGUAACUUAGAAGUAACACACACCGAUCAACAGUCAGCGUGGCACACCAGCCACGUUUUG | |
| | AUCAAGCACUUCUGUUACCCCGGACUGAGUAUCAAUAGACUGCUCACGCGGUUGAAGGAGAAAGCGU | |
| | UCGUUAUCCGGCCAACUACUUCGAAAAACCUAGUAACACCGUGGAAGUUGCAGAGUGUUUCGCUCAG | |
| | CACUACCCCAGUGUAGAUCAGGUCGAUGAGUCACCGCAUUCCCCACGGGCGACCGUGGCGGUGGCUG | |
| | CGUUGGCGGCCUGCCCAUGGGGAAACCCAUGGGACGCUCUAAUACAGACAUGGUGCGAAGAGUCUAU | |
| | UGAGCUAGUUGGUAGUCCUCCGGCCCCUGAAUGCGGCUAAUCCUAACUGCGGAGCACACACCCUCAA | |
| | GCCAGAGGGCAGUGUGUCGUAACGGGCAACUCUGCAGCGGAACCGACUACUUUGGGUGUCCGUGUUU | |
| | CAUUUUAUUCCUAUACUGGCUGCUUAUGGUGACAAUUGAGAGAUCGUUACCAUAUAGCUAUUGGAUU | |
| | GGCCAUCCGGUGACUAAUAGAGCUAUUAUAUAUCCCUUUGUUGGGUUUAUACCACUUAGCUUGAAAG | |
| | AGGUUAAAACAUUACAAUUCAUUGUUAAGUUGAAUACAGCAAAGCCACCAUGUAUAGAAUGCAGCUG | |
| | CUGUCUUGCAUCGCCCUGAGCCUGGCCCUGGUCACAAACAGCGCUCCUACAAGCUCUAGCACCAAGA | |
| | AAACCCAGCUGCAGCUCGAGCACCUGCUGCUGGACCUGCAGAUGAUCCUGAACGGCAUUAACAACUA | |
| | CAAGAACCCAAAGCUGACCAGAAUGCUGACAUUUAAGUUCUACAUGCCUAAGAAGGCCACAGAGCUG | |
| | AAGCACCUGCAAUGUCUGGAAGAGGAACUGAAGCCCCUGGAAGAGGUGCUUAAUCUGGCUCAGAGCA | |
| | AGAACUUCCACCUGAGACCUCGGGACCUGAUCAGCGACAUCAACGUGAUCGUGCUGGAACUGAAAGG | |
| | CAGCGAGACAACCUUCAUGUGCGAGUACGCCGAUGAGACCGCCACCAUCGUGGAGUUCCUGAAUCGG | |
| | UGGAUCACCUUCUGCCAGUCCAUCAUCUCCACCCUGACCUGAUAAAGACGGGGGGGGCUUAAAUAAU | |
| | UGAGCCUUAAAGAAGAAAUUCUUUAAGUGGAUGCUCUCAAACUCAGGGAAACCUAAAUCUAGUUAUA | |
| | GACAAGGCAAUCCUGAGCCAAGCCGAAGUAGUAAUUAGUAACAUCUCUACUGAAAAAUACAAAAAAU | |
| | UAGCCGGGCGUAGUGGCAUGAGCCUGUAGUCCCAGCUACUUGGGAGGCUGAGGCAGGAGAAUGGCGU | |
| | GAACUCGGGAGGCAGAGCUUGCAGUGAGCCGAGAUCACACCACUGCACUCCAGCCUGGGCGACAGAG | |
| | CGAGACUCCGUCUCAUAACAGCAUAUCUAG | |
| 540 | GGGAGACCCUCGAAGAGAGUCUCGCUCUUCGCCCAGGCUGGAGUGAGUGGUGUGAUCUGGCUCACUG | AnaX-MEHA4- |
| | CAACUCUCCUCCCGUCAGCAUUCUCUGCCUCAGCCUCCCAGUAGCUGGAUACAGGCCGCCACACCCG | CVB3-IL2 |
| | CUAUUUUUGUAUUUUCAGUAGAGAUGAACAAUAGAUGACUUACAACUAAUCGGAAGGUGCAGAGACU | precursor |
| | CGACGGGAGCUACCCUAACGUCAAGACGAGGGUAAAGAGAGAGUCCAAUUCUCAAAGCCAAUAGGCA | |
| | GUAGCGAAAGCUGCAAGAGAAUGAAAACCCCCCCAUUAAAACAGCCUGUGGGUUGAUCCCACCCACA | |
| | GGCCCAUUGGGCGCUAGCACUCUGGUAUCACGGUACCUUUGUGCGCCUGUUUUAUACCCCCUCCCCC | |
| | AACUGUAACUUAGAAGUAACACACACCGAUCAACAGUCAGCGUGGCACACCAGCCACGUUUUGAUCA | |
| | AGCACUUCUGUUACCCCGGACUGAGUAUCAAUAGACUGCUCACGCGGUUGAAGGAGAAAGCGUUCGU | |
| | UAUCCGGCCAACUACUUCGAAAAACCUAGUAACACCGUGGAAGUUGCAGAGUGUUUCGCUCAGCACU | |
| | ACCCCAGUGUAGAUCAGGUCGAUGAGUCACCGCAUUCCCCACGGGCGACCGUGGCGGUGGCUGCGUU | |
| | GGCGGCCUGCCCAUGGGGAAACCCAUGGGACGCUCUAAUACAGACAUGGUGCGAAGAGUCUAUUGAG | |
| | CUAGUUGGUAGUCCUCCGGCCCCUGAAUGCGGCUAAUCCUAACUGCGGAGCACACACCCUCAAGCCA | |
| | GAGGGCAGUGUGUCGUAACGGGCAACUCUGCAGCGGAACCGACUACUUUGGGUGUCCGUGUUUCAUU | |
| | UUAUUCCUAUACUGGCUGCUUAUGGUGACAAUUGAGAGAUCGUUACCAUAUAGCUAUUGGAUUGGCC | |
| | AUCCGGUGACUAAUAGAGCUAUUAUAUAUCCCUUUGUUGGGUUUAUACCACUUAGCUUGAAAGAGGU | |
| | UAAAACAUUACAAUUCAUUGUUAAGUUGAAUACAGCAAAGCCACCAUGUAUAGAAUGCAGCUGCUGU | |
| | CUUGCAUCGCCCUGAGCCUGGCCCUGGUCACAAACAGCGCUCCUACAAGCUCUAGCACCAAGAAAAC | |
| | CCAGCUGCAGCUCGAGCACCUGCUGCUGGACCUGCAGAUGAUCCUGAACGGCAUUAACAACUACAAG | |
| | AACCCAAAGCUGACCAGAAUGCUGACAUUUAAGUUCUACAUGCCUAAGAAGGCCACAGAGCUGAAGC | |
| | ACCUGCAAUGUCUGGAAGAGGAACUGAAGCCCCUGGAAGAGGUGCUUAAUCUGGCUCAGAGCAAGAA | |
| | CUUCCACCUGAGACCUCGGGACCUGAUCAGCGACAUCAACGUGAUCGUGCUGGAACUGAAAGGCAGC | |
| | GAGACAACCUUCAUGUGCGAGUACGCCGAUGAGACCGCCACCAUCGUGGAGUUCCUGAAUCGGUGGA | |
| | UCACCUUCUGCCAGUCCAUCAUCUCCACCCUGACCUGAUAAAGACGGGGGGGGCUUAAAUAAUUGAG | |
| | CCUUAAAGAAGAAAUUCUUUAAGUGGAUGCUCUCAAACUCAGGGAAACCUAAAUCUAGUUAUAGACA | |
| | AGGCAAUCCUGAGCCAAGCCGAAGUAGUAAUUAGUAACAUCUCUACUGAAAAUACAAAAAUAGCGGG | |
| | UGUGGCGGCCUGUAUCCAGCUACUGGGAGGCUGAGGCAGAGAAUGCUGACGGGAGGAGAGUUGCAGU | |
| | GAGCCAGAUCACACCACUCACUCCAGCCUGGGCGAAGAGCGAGACUCUCUAUAACAGCAUAUCUAG | |
| 541 | GGGAGACCCUCGAGGCCCCUCUGCAGUGUCUGUUGUGGAGGCCAGAUACGUAACUGCUUCCGCAACA | AnaX-MEHA5- |
| | AUAGAUGACUUACAACUAAUCGGAAGGUGCAGAGACUCGACGGGAGCUACCCUAACGUCAAGACGAG | CVB3-IL2 |
| | GGUAAAGAGAGAGUCCAAUUCUCAAAGCCAAUAGGCAGUAGCGAAAGCUGCAAGAGAAUGAAAACCC | precursor |
| | CCCCAUUAAAACAGCCUGUGGGUUGAUCCCACCCACAGGCCCAUUGGGCGCUAGCACUCUGGUAUCA | |
| | CGGUACCUUUGUGCGCCUGUUUUAUACCCCCUCCCCCAACUGUAACUUAGAAGUAACACACACCGAU | |
| | CAACAGUCAGCGUGGCACACCAGCCACGUUUUGAUCAAGCACUUCUGUUACCCCGGACUGAGUAUCA | |
| | AUAGACUGCUCACGCGGUUGAAGGAGAAAGCGUUCGUUAUCCGGCCAACUACUUCGAAAAACCUAGU | |
| | AACACCGUGGAAGUUGCAGAGUGUUUCGCUCAGCACUACCCCAGUGUAGAUCAGGUCGAUGAGUCAC | |
| | CGCAUUCCCCACGGGCGACCGUGGCGGUGGCUGCGUUGGCGGCCUGCCCAUGGGGAAACCCAUGGGA | |
| | CGCUCUAAUACAGACAUGGUGCGAAGAGUCUAUUGAGCUAGUUGGUAGUCCUCCGGCCCCUGAAUGC | |
| | GGCUAAUCCUAACUGCGGAGCACACACCCUCAAGCCAGAGGGCAGUGUGUCGUAACGGGCAACUCUG | |
| | CAGCGGAACCGACUACUUUGGGUGUCCGUGUUUCAUUUUAUUCCUAUACUGGCUGCUUAUGGUGACA | |
| | AUUGAGAGAUCGUUACCAUAUAGCUAUUGGAUUGGCCAUCCGGUGACUAAUAGAGCUAUUAUAUAUC | |
| | CCUUUGUUGGGUUUAUACCACUUAGCUUGAAAGAGGUUAAAACAUUACAAUUCAUUGUUAAGUUGAA | |
| | UACAGCAAAGCCACCAUGUAUAGAAUGCAGCUGCUGUCUUGCAUCGCCCUGAGCCUGGCCCUGGUCA | |
| | CAAACAGCGCUCCUACAAGCUCUAGCACCAAGAAAACCCAGCUGCAGCUCGAGCACCUGCUGCUGGA | |
| | CCUGCAGAUGAUCCUGAACGGCAUUAACAACUACAAGAACCCAAAGCUGACCAGAAUGCUGACAUUU | |
| | AAGUUCUACAUGCCUAAGAAGGCCACAGAGCUGAAGCACCUGCAAUGUCUGGAAGAGGAACUGAAGC | |
| | CCCUGGAAGAGGUGCUUAAUCUGGCUCAGAGCAAGAACUUCCACCUGAGACCUCGGGACCUGAUCAG | |
| | CGACAUCAACGUGAUCGUGCUGGAACUGAAAGGCAGCGAGACAACCUUCAUGUGCGAGUACGCCGAU | |
| | GAGACCGCCACCAUCGUGGAGUUCCUGAAUCGGUGGAUCACCUUCUGCCAGUCCAUCAUCUCCACCC | |
| | UGACCUGAUAAAGACGGGGGGGGCUUAAAUAAUUGAGCCUUAAAGAAGAAAUUCUUUAAGUGGAUGC | |
| | UCUCAAACUCAGGGAAACCUAAAUCUAGUUAUAGACAAGGCAAUCCUGAGCCAAGCCGAAGUAGUAA | |
| | UUAGUAAGCGGAAGCAGUUACGUAUCUGGCCUCCACAACAGACACUGCAGAGGGGCCAUAACAGCAU | |
| | AUCUAG | |
| 542 | GGGAGACCCUCGAUAGACUUUGAGCAGAAAGAGGUUGAAAAUGUUGAGCCUGAUUUCUCUUAGAACA | AnaX-MEHA6- |
| | AUAGAUGACUUACAACUAAUCGGAAGGUGCAGAGACUCGACGGGAGCUACCCUAACGUCAAGACGAG | CVB3-IL2 |
| | GGUAAAGAGAGAGUCCAAUUCUCAAAGCCAAUAGGCAGUAGCGAAAGCUGCAAGAGAAUGAAAACCC | precursor |
| | CCCCAUUAAAACAGCCUGUGGGUUGAUCCCACCCACAGGCCCAUUGGGCGCUAGCACUCUGGUAUCA | |
| | CGGUACCUUUGUGCGCCUGUUUUAUACCCCCUCCCCCAACUGUAACUUAGAAGUAACACACACCGAU | |
| | CAACAGUCAGCGUGGCACACCAGCCACGUUUUGAUCAAGCACUUCUGUUACCCCGGACUGAGUAUCA | |
| | AUAGACUGCUCACGCGGUUGAAGGAGAAAGCGUUCGUUAUCCGGCCAACUACUUCGAAAAACCUAGU | |
| | AACACCGUGGAAGUUGCAGAGUGUUUCGCUCAGCACUACCCCAGUGUAGAUCAGGUCGAUGAGUCAC | |
| | CGCAUUCCCCACGGGCGACCGUGGCGGUGGCUGCGUUGGCGGCCUGCCCAUGGGGAAACCCAUGGGA | |
| | CGCUCUAAUACAGACAUGGUGCGAAGAGUCUAUUGAGCUAGUUGGUAGUCCUCCGGCCCCUGAAUGC | |
| | GGCUAAUCCUAACUGCGGAGCACACACCCUCAAGCCAGAGGGCAGUGUGUCGUAACGGGCAACUCUG | |
| | CAGCGGAACCGACUACUUUGGGUGUCCGUGUUUCAUUUUAUUCCUAUACUGGCUGCUUAUGGUGACA | |
| | AUUGAGAGAUCGUUACCAUAUAGCUAUUGGAUUGGCCAUCCGGUGACUAAUAGAGCUAUUAUAUAUC | |
| | CCUUUGUUGGGUUUAUACCACUUAGCUUGAAAGAGGUUAAAACAUUACAAUUCAUUGUUAAGUUGAA | |
| | UACAGCAAAGCCACCAUGUAUAGAAUGCAGCUGCUGUCUUGCAUCGCCCUGAGCCUGGCCCUGGUCA | |
| | CAAACAGCGCUCCUACAAGCUCUAGCACCAAGAAAACCCAGCUGCAGCUCGAGCACCUGCUGCUGGA | |
| | CCUGCAGAUGAUCCUGAACGGCAUUAACAACUACAAGAACCCAAAGCUGACCAGAAUGCUGACAUUU | |
| | AAGUUCUACAUGCCUAAGAAGGCCACAGAGCUGAAGCACCUGCAAUGUCUGGAAGAGGAACUGAAGC | |
| | CCCUGGAAGAGGUGCUUAAUCUGGCUCAGAGCAAGAACUUCCACCUGAGACCUCGGGACCUGAUCAG | |
| | CGACAUCAACGUGAUCGUGCUGGAACUGAAAGGCAGCGAGACAACCUUCAUGUGCGAGUACGCCGAU | |
| | GAGACCGCCACCAUCGUGGAGUUCCUGAAUCGGUGGAUCACCUUCUGCCAGUCCAUCAUCUCCACCC | |
| | UGACCUGAUAAAGACGGGGGGGGCUUAAAUAAUUGAGCCUUAAAGAAGAAAUUCUUUAAGUGGAUGC | |
| | UCUCAAACUCAGGGAAACCUAAAUCUAGUUAUAGACAAGGCAAUCCUGAGCCAAGCCGAAGUAGUAA | |
| | UUAGUAACUAAGAGAAAUCAGGCUCAACAUUUUCAACCUCUUUCUGCUCAAAGUCUAAUAACAGCAU | |
| | AUCUAG | |
| 543 | AAAAUCCGUUGAUUAAAACAGCCUGUGGGUUGAUCCCACCCACAGGCCCAUUGGGCGCUAGCACUCU | AnaX-CVB3- |
| | GGUAUCACGGUACCUUUGUGCGCCUGUUUUAUACCCCCUCCCCCAACUGUAACUUAGAAGUAACACA | IL2 circRNA |
| | CACCGAUCAACAGUCAGCGUGGCACACCAGCCACGUUUUGAUCAAGCACUUCUGUUACCCCGGACUG | |
| | AGUAUCAAUAGACUGCUCACGCGGUUGAAGGAGAAAGCGUUCGUUAUCCGGCCAACUACUUCGAAAA | |
| | ACCUAGUAACACCGUGGAAGUUGCAGAGUGUUUCGCUCAGCACUACCCCAGUGUAGAUCAGGUCGAU | |
| | GAGUCACCGCAUUCCCCACGGGCGACCGUGGCGGUGGCUGCGUUGGCGGCCUGCCCAUGGGGAAACC | |
| | CAUGGGACGCUCUAAUACAGACAUGGUGCGAAGAGUCUAUUGAGCUAGUUGGUAGUCCUCCGGCCCC | |
| | UGAAUGCGGCUAAUCCUAACUGCGGAGCACACACCCUCAAGCCAGAGGGCAGUGUGUCGUAACGGGC | |
| | AACUCUGCAGCGGAACCGACUACUUUGGGUGUCCGUGUUUCAUUUUAUUCCUAUACUGGCUGCUUAU | |
| | GGUGACAAUUGAGAGAUCGUUACCAUAUAGCUAUUGGAUUGGCCAUCCGGUGACUAAUAGAGCUAUU | |
| | AUAUAUCCCUUUGUUGGGUUUAUACCACUUAGCUUGAAAGAGGUUAAAACAUUACAAUUCAUUGUUA | |
| | AGUUGAAUACAGCAAAAUGUAUAGAAUGCAGCUGCUGUCUUGCAUCGCCCUGAGCCUGGCCCUGGUC | |
| | ACAAACAGCGCUCCUACAAGCUCUAGCACCAAGAAAACCCAGCUGCAGCUCGAGCACCUGCUGCUGG | |
| | ACCUGCAGAUGAUCCUGAACGGCAUUAACAACUACAAGAACCCAAAGCUGACCAGAAUGCUGACAUU | |
| | UAAGUUCUACAUGCCUAAGAAGGCCACAGAGCUGAAGCACCUGCAAUGUCUGGAAGAGGAACUGAAG | |
| | CCCCUGGAAGAGGUGCUUAAUCUGGCUCAGAGCAAGAACUUCCACCUGAGACCUCGGGACCUGAUCA | |
| | GCGACAUCAACGUGAUCGUGCUGGAACUGAAAGGCAGCGAGACAACCUUCAUGUGCGAGUACGCCGA | |
| | UGAGACCGCCACCAUCGUGGAGUUCCUGAAUCGGUGGAUCACCUUCUGCCAGUCCAUCAUCUCCACC | |
| | CUGACCUGAUAAUAGAGACGCUACGGACUU | |
| 544 | AGAAGUAACACACACCGAUCAACAGUCAGCGUGGCACACCAGCCACGUUUUGAUCAAGCACUUCUGU | AnaX-CLEAN- |
| | UACCCCGGACUGAGUAUCAAUAGACUGCUCACGCGGUUGAAGGAGAAAGCGUUCGUUAUCCGGCCAA | PIE-IRES- |
| | CUACUUCGAAAAACCUAGUAACACCGUGGAAGUUGCAGAGUGUUUCGCUCAGCACUACCCCAGUGUA | rawCVB3- |
| | GAUCAGGUCGAUGAGUCACCGCAUUCCCCACGGGCGACCGUGGCGGUGGCUGCGUUGGCGGCCUGCC | CVB3-IL2 |
| | CAUGGGGAAACCCAUGGGACGCUCUAAUACAGACAUGGUGCGAAGAGUCUAUUGAGCUAGUUGGUAG | circRNA |
| | UCCUCCGGCCCCUGAAUGCGGCUAAUCCUAACUGCGGAGCACACACCCUCAAGCCAGAGGGCAGUGU | |
| | GUCGUAACGGGCAACUCUGCAGCGGAACCGACUACUUUGGGUGUCCGUGUUUCAUUUUAUUCCUAUA | |
| | CUGGCUGCUUAUGGUGACAAUUGAGAGAUCGUUACCAUAUAGCUAUUGGAUUGGCCAUCCGGUGACU | |
| | AAUAGAGCUAUUAUAUAUCCCUUUGUUGGGUUUAUACCACUUAGCUUGAAAGAGGUUAAAACAUUAC | |
| | AAUUCAUUGUUAAGUUGAAUACAGCAAAGCCACCAUGUAUAGAAUGCAGCUGCUGUCUUGCAUCGCC | |
| | CUGAGCCUGGCCCUGGUCACAAACAGCGCUCCUACAAGCUCUAGCACCAAGAAAACCCAGCUGCAGC | |
| | UCGAGCACCUGCUGCUGGACCUGCAGAUGAUCCUGAACGGCAUUAACAACUACAAGAACCCAAAGCU | |
| | GACCAGAAUGCUGACAUUUAAGUUCUACAUGCCUAAGAAGGCCACAGAGCUGAAGCACCUGCAAUGU | |
| | CUGGAAGAGGAACUGAAGCCCCUGGAAGAGGUGCUUAAUCUGGCUCAGAGCAAGAACUUCCACCUGA | |
| | GACCUCGGGACCUGAUCAGCGACAUCAACGUGAUCGUGCUGGAACUGAAAGGCAGCGAGACAACCUU | |
| | CAUGUGCGAGUACGCCGAUGAGACCGCCACCAUCGUGGAGUUCCUGAAUCGGUGGAUCACCUUCUGC | |
| | CAGUCCAUCAUCUCCACCCUGACCUGAUAAUUAAAACAGCCUGUGGGUUGAUCCCACCCACAGGCCC | |
| | AUUGGGCGCUAGCACUCUGGUAUCACGGUACCUUUGUGCGCCUGUUUUAUACCCCCUCCCCCAACUG | |
| | UAACUU | |
| 545 | AAAAUUACAACACACCGAUCAACAGUCAGCGUGGCACACCAGCCACGUUUUGAUCAAGCACUUCUGU | AnaX-CLEAN- |
| | UACCCCGGACUGAGUAUCAAUAGACUGCUCACGCGGUUGAAGGAGAAAGCGUUCGUUAUCCGGCCAA | PIE-IRES- |
| | CUACUUCGAAAAACCUAGUAACACCGUGGAAGUUGCAGAGUGUUUCGCUCAGCACUACCCCAGUGUA | rawMutation2- |
| | GAUCAGGUCGAUGAGUCACCGCAUUCCCCACGGGCGACCGUGGCGGUGGCUGCGUUGGCGGCCUGCC | CVB3-IL2 |
| | CAUGGGGAAACCCAUGGGACGCUCUAAUACAGACAUGGUGCGAAGAGUCUAUUGAGCUAGUUGGUAG | circRNA |
| | UCCUCCGGCCCCUGAAUGCGGCUAAUCCUAACUGCGGAGCACACACCCUCAAGCCAGAGGGCAGUGU | |
| | GUCGUAACGGGCAACUCUGCAGCGGAACCGACUACUUUGGGUGUCCGUGUUUCAUUUUAUUCCUAUA | |
| | CUGGCUGCUUAUGGUGACAAUUGAGAGAUCGUUACCAUAUAGCUAUUGGAUUGGCCAUCCGGUGACU | |
| | AAUAGAGCUAUUAUAUAUCCCUUUGUUGGGUUUAUACCACUUAGCUUGAAAGAGGUUAAAACAUUAC | |
| | AAUUCAUUGUUAAGUUGAAUACAGCAAAGCCACCAUGUAUAGAAUGCAGCUGCUGUCUUGCAUCGCC | |
| | CUGAGCCUGGCCCUGGUCACAAACAGCGCUCCUACAAGCUCUAGCACCAAGAAAACCCAGCUGCAGC | |
| | UCGAGCACCUGCUGCUGGACCUGCAGAUGAUCCUGAACGGCAUUAACAACUACAAGAACCCAAAGCU | |
| | GACCAGAAUGCUGACAUUUAAGUUCUACAUGCCUAAGAAGGCCACAGAGCUGAAGCACCUGCAAUGU | |
| | CUGGAAGAGGAACUGAAGCCCCUGGAAGAGGUGCUUAAUCUGGCUCAGAGCAAGAACUUCCACCUGA | |
| | GACCUCGGGACCUGAUCAGCGACAUCAACGUGAUCGUGCUGGAACUGAAAGGCAGCGAGACAACCUU | |
| | CAUGUGCGAGUACGCCGAUGAGACCGCCACCAUCGUGGAGUUCCUGAAUCGGUGGAUCACCUUCUGC | |
| | CAGUCCAUCAUCUCCACCCUGACCUGAUAAUUAAAACAGCCUGUGGGUUGAUCCCACCCACAGGCCC | |
| | AUUGGGCGCUAGCACUCUGGUAUCACGGUACCUUUGUGCGCCUGUUUUAUACCCCCUCCCCCAACUG | |
| | UAACUU | |
| 546 | AAACCUCGCUCAGAGCAAGAACUUCCACCUGAGACCUCGGGACCUGAUCAGCGACAUCAACGUGAUC | AnaX-CLEAN- |
| | GUGCUGGAACUGAAAGGCAGCGAGACAACCUUCAUGUGCGAGUACGCCGAUGAGACCGCCACCAUCG | PIE-Cargo- |
| | UGGAGUUCCUGAAUCGGUGGAUCACCUUCUGCCAGUCCAUCAUCUCCACCCUGACCUGAUAAUUAAA | PIE1-CVB3- |
| | ACAGCCUGUGGGUUGAUCCCACCCACAGGCCCAUUGGGCGCUAGCACUCUGGUAUCACGGUACCUUU | IL2 circRNA |
| | GUGCGCCUGUUUUAUACCCCCUCCCCCAACUGUAACUUAGAAGUAACACACACCGAUCAACAGUCAG | |
| | CGUGGCACACCAGCCACGUUUUGAUCAAGCACUUCUGUUACCCCGGACUGAGUAUCAAUAGACUGCU | |
| | CACGCGGUUGAAGGAGAAAGCGUUCGUUAUCCGGCCAACUACUUCGAAAAACCUAGUAACACCGUGG | |
| | AAGUUGCAGAGUGUUUCGCUCAGCACUACCCCAGUGUAGAUCAGGUCGAUGAGUCACCGCAUUCCCC | |
| | ACGGGCGACCGUGGCGGUGGCUGCGUUGGCGGCCUGCCCAUGGGGAAACCCAUGGGACGCUCUAAUA | |
| | CAGACAUGGUGCGAAGAGUCUAUUGAGCUAGUUGGUAGUCCUCCGGCCCCUGAAUGCGGCUAAUCCU | |
| | AACUGCGGAGCACACACCCUCAAGCCAGAGGGCAGUGUGUCGUAACGGGCAACUCUGCAGCGGAACC | |
| | GACUACUUUGGGUGUCCGUGUUUCAUUUUAUUCCUAUACUGGCUGCUUAUGGUGACAAUUGAGAGAU | |
| | CGUUACCAUAUAGCUAUUGGAUUGGCCAUCCGGUGACUAAUAGAGCUAUUAUAUAUCCCUUUGUUGG | |
| | GUUUAUACCACUUAGCUUGAAAGAGGUUAAAACAUUACAAUUCAUUGUUAAGUUGAAUACAGCAAAG | |
| | CCACCAUGUAUAGAAUGCAGCUGCUGUCUUGCAUCGCCCUGAGCCUGGCCCUGGUCACAAACAGCGC | |
| | UCCUACAAGCUCUAGCACCAAGAAAACCCAGCUGCAGCUCGAGCACCUGCUGCUGGACCUGCAGAUG | |
| | AUCCUGAACGGCAUUAACAACUACAAGAACCCAAAGCUGACCAGAAUGCUGACAUUUAAGUUCUACA | |
| | UGCCUAAGAAGGCCACAGAGCUGAAGCACCUGCAAUGUCUGGAAGAGGAACUGAAGCCCCUGGAAGA | |
| | GGUCUU | |
| 547 | AAUCUCUGACAUCAACGUGAUCGUGCUGGAACUGAAAGGCAGCGAGACAACCUUCAUGUGCGAGUAC | AnaX-CLEAN- |
| | GCCGAUGAGACCGCCACCAUCGUGGAGUUCCUGAAUCGGUGGAUCACCUUCUGCCAGUCCAUCAUCU | PIE-Cargo- |
| | CCACCCUGACCUGAUAAUUAAAACAGCCUGUGGGUUGAUCCCACCCACAGGCCCAUUGGGCGCUAGC | PIE2-CVB3- |
| | ACUCUGGUAUCACGGUACCUUUGUGCGCCUGUUUUAUACCCCCUCCCCCAACUGUAACUUAGAAGUA | IL2 circRNA |
| | ACACACACCGAUCAACAGUCAGCGUGGCACACCAGCCACGUUUUGAUCAAGCACUUCUGUUACCCCG | |
| | GACUGAGUAUCAAUAGACUGCUCACGCGGUUGAAGGAGAAAGCGUUCGUUAUCCGGCCAACUACUUC | |
| | GAAAAACCUAGUAACACCGUGGAAGUUGCAGAGUGUUUCGCUCAGCACUACCCCAGUGUAGAUCAGG | |
| | UCGAUGAGUCACCGCAUUCCCCACGGGCGACCGUGGCGGUGGCUGCGUUGGCGGCCUGCCCAUGGGG | |
| | AAACCCAUGGGACGCUCUAAUACAGACAUGGUGCGAAGAGUCUAUUGAGCUAGUUGGUAGUCCUCCG | |
| | GCCCCUGAAUGCGGCUAAUCCUAACUGCGGAGCACACACCCUCAAGCCAGAGGGCAGUGUGUCGUAA | |
| | CGGGCAACUCUGCAGCGGAACCGACUACUUUGGGUGUCCGUGUUUCAUUUUAUUCCUAUACUGGCUG | |
| | CUUAUGGUGACAAUUGAGAGAUCGUUACCAUAUAGCUAUUGGAUUGGCCAUCCGGUGACUAAUAGAG | |
| | CUAUUAUAUAUCCCUUUGUUGGGUUUAUACCACUUAGCUUGAAAGAGGUUAAAACAUUACAAUUCAU | |
| | UGUUAAGUUGAAUACAGCAAAGCCACCAUGUAUAGAAUGCAGCUGCUGUCUUGCAUCGCCCUGAGCC | |
| | UGGCCCUGGUCACAAACAGCGCUCCUACAAGCUCUAGCACCAAGAAAACCCAGCUGCAGCUCGAGCA | |
| | CCUGCUGCUGGACCUGCAGAUGAUCCUGAACGGCAUUAACAACUACAAGAACCCAAAGCUGACCAGA | |
| | AUGCUGACAUUUAAGUUCUACAUGCCUAAGAAGGCCACAGAGCUGAAGCACCUGCAAUGUCUGGAAG | |
| | AGGAACUGAAGCCCCUGGAAGAGGUGCUUAAUCUGGCUCAGAGCAAGAACUUCCACCUGAGACCUAG | |
| | AGACUU | |
| 548 | AAAACCCCCCCAUUAAAACAGCCUGUGGGUUGAUCCCACCCACAGGCCCAUUGGGCGCUAGCACUCU | AnaX-GCv3- |
| | GGUAUCACGGUACCUUUGUGCGCCUGUUUUAUACCCCCUCCCCCAACUGUAACUUAGAAGUAACACA | 2 × homo-CVB3- |
| | CACCGAUCAACAGUCAGCGUGGCACACCAGCCACGUUUUGAUCAAGCACUUCUGUUACCCCGGACUG | AQP1 circRNA |
| | AGUAUCAAUAGACUGCUCACGCGGUUGAAGGAGAAAGCGUUCGUUAUCCGGCCAACUACUUCGAAAA | |
| | ACCUAGUAACACCGUGGAAGUUGCAGAGUGUUUCGCUCAGCACUACCCCAGUGUAGAUCAGGUCGAU | |
| | GAGUCACCGCAUUCCCCACGGGCGACCGUGGCGGUGGCUGCGUUGGCGGCCUGCCCAUGGGGAAACC | |
| | CAUGGGACGCUCUAAUACAGACAUGGUGCGAAGAGUCUAUUGAGCUAGUUGGUAGUCCUCCGGCCCC | |
| | UGAAUGCGGCUAAUCCUAACUGCGGAGCACACACCCUCAAGCCAGAGGGCAGUGUGUCGUAACGGGC | |
| | AACUCUGCAGCGGAACCGACUACUUUGGGUGUCCGUGUUUCAUUUUAUUCCUAUACUGGCUGCUUAU | |
| | GGUGACAAUUGAGAGAUCGUUACCAUAUAGCUAUUGGAUUGGCCAUCCGGUGACUAAUAGAGCUAUU | |
| | AUAUAUCCCUUUGUUGGGUUUAUACCACUUAGCUUGAAAGAGGUUAAAACAUUACAAUUCAUUGUUA | |
| | AGUUGAAUACAGCAAAGCCACCAUGGCCAGCGAGUUCAAGAAGAAGCUCUUCUGGAGGGCAGUGGUG | |
| | GCCGAGUUCCUGGCCACGACCCUCUUUGUCUUCAUCAGCAUCGGUUCUGCCCUGGGCUUCAAAUACC | |
| | CGGUGGGGAACAACCAGACGGCGGUCCAGGACAACGUGAAGGUGUCGCUGGCCUUCGGGCUGAGCAU | |
| | CGCCACGCUGGCGCAGAGUGUGGGCCACAUCAGCGGCGCCCACCUCAACCCGGCUGUCACACUGGGG | |
| | CUGCUGCUCAGCUGCCAGAUCAGCAUCUUCCGUGCCCUCAUGUACAUCAUCGCCCAGUGCGUGGGGG | |
| | CCAUCGUCGCCACCGCCAUCCUCUCAGGCAUCACCUCCUCCCUGACUGGGAACUCGCUUGGCCGCAA | |
| | UGACCUGGCUGAUGGUGUGAACUCGGGCCAGGGCCUGGGCAUCGAGAUCAUCGGGACCCUCCAGCUG | |
| | GUGCUAUGCGUGCUGGCUACUACCGACCGGAGGCGCCGUGACCUUGGUGGCUCAGCCCCCCUUGCCA | |
| | UCGGCCUCUCUGUAGCCCUUGGACACCUCCUGGCUAUUGACUACACUGGCUGUGGGAUUAACCCUGC | |
| | UCGGUCCUUUGGCUCCGCGGUGAUCACACACAACUUCAGCAACCACUGGAUUUUCUGGGUGGGGCCA | |
| | UUCAUCGGGGGAGCCCUGGCUGUACUCAUCUACGACUUCAUCCUGGCCCCACGCAGCAGUGACCUCA | |
| | CAGACCGCGUGAAGGUGUGGACCAGCGGCCAGGUGGAGGAGUAUGACCUGGAUGCCGACGACAUCAA | |
| | CUCCAGGGUGGAGAUGAAGCCCAAAUGAUAAAGACGGGGGGGGCUU | |
| 549 | AACUGGAAACUCGCUUGGCCGCAAUGACCUGGCUGAUGGUGUGAACUCGGGCCAGGGCCUGGGCAUC | AnaX-GCv3- |
| | GAGAUCAUCGGGACCCUCCAGCUGGUGCUAUGCGUGCUGGCUACUACCGACCGGAGGCGCCGUGACC | 2 × homo- |
| | UUGGUGGCUCAGCCCCCCUUGCCAUCGGCCUCUCUGUAGCCCUUGGACACCUCCUGGCUAUUGACUA | CLEAN-PIE- |
| | CACUGGCUGUGGGAUUAACCCUGCUCGGUCCUUUGGCUCCGCGGUGAUCACACACAACUUCAGCAAC | Cargo-PIE1- |
| | CACUGGAUUUUCUGGGUGGGGCCAUUCAUCGGGGGAGCCCUGGCUGUACUCAUCUACGACUUCAUCC | CVB3-AQP1 |
| | UGGCCCCACGCAGCAGUGACCUCACAGACCGCGUGAAGGUGUGGACCAGCGGCCAGGUGGAGGAGUA | circRNA |
| | UGACCUGGAUGCCGACGACAUCAACUCCAGGGUGGAGAUGAAGCCCAAAUGAUAAUUAAAACAGCCU | |
| | GUGGGUUGAUCCCACCCACAGGCCCAUUGGGCGCUAGCACUCUGGUAUCACGGUACCUUUGUGCGCC | |
| | UGUUUUAUACCCCCUCCCCCAACUGUAACUUAGAAGUAACACACACCGAUCAACAGUCAGCGUGGCA | |
| | CACCAGCCACGUUUUGAUCAAGCACUUCUGUUACCCCGGACUGAGUAUCAAUAGACUGCUCACGCGG | |
| | UUGAAGGAGAAAGCGUUCGUUAUCCGGCCAACUACUUCGAAAAACCUAGUAACACCGUGGAAGUUGC | |
| | AGAGUGUUUCGCUCAGCACUACCCCAGUGUAGAUCAGGUCGAUGAGUCACCGCAUUCCCCACGGGCG | |
| | ACCGUGGCGGUGGCUGCGUUGGCGGCCUGCCCAUGGGGAAACCCAUGGGACGCUCUAAUACAGACAU | |
| | GGUGCGAAGAGUCUAUUGAGCUAGUUGGUAGUCCUCCGGCCCCUGAAUGCGGCUAAUCCUAACUGCG | |
| | GAGCACACACCCUCAAGCCAGAGGGCAGUGUGUCGUAACGGGCAACUCUGCAGCGGAACCGACUACU | |
| | UUGGGUGUCCGUGUUUCAUUUUAUUCCUAUACUGGCUGCUUAUGGUGACAAUUGAGAGAUCGUUACC | |
| | AUAUAGCUAUUGGAUUGGCCAUCCGGUGACUAAUAGAGCUAUUAUAUAUCCCUUUGUUGGGUUUAUA | |
| | CCACUUAGCUUGAAAGAGGUUAAAACAUUACAAUUCAUUGUUAAGUUGAAUACAGCAAAGCCACCAU | |
| | GGCCAGCGAGUUCAAGAAGAAGCUCUUCUGGAGGGCAGUGGUGGCCGAGUUCCUGGCCACGACCCUC | |
| | UUUGUCUUCAUCAGCAUCGGUUCUGCCCUGGGCUUCAAAUACCCGGUGGGGAACAACCAGACGGCGG | |
| | UCCAGGACAACGUGAAGGUGUCGCUGGCCUUCGGGCUGAGCAUCGCCACGCUGGCGCAGAGUGUGGG | |
| | CCACAUCAGCGGCGCCCACCUCAACCCGGCUGUCACACUGGGGCUGCUGCUCAGCUGCCAGAUCAGC | |
| | AUCUUCCGUGCCCUCAUGUACAUCAUCGCCCAGUGCGUGGGGGCCAUCGUCGCCACCGCCAUCCUCU | |
| | CAGGCAUCACCUCCAGCUU | |
| 550 | AACCAGCUCCCUGACUGGGAACUCGCUUGGCCGCAAUGACCUGGCUGAUGGUGUGAACUCGGGCCAG | Twort-PIE-6- |
| | GGCCUGGGCAUCGAGAUCAUCGGGACCCUCCAGCUGGUGCUAUGCGUGCUGGCUACUACCGACCGGA | CLEAN-PIE- |
| | GGCGCCGUGACCUUGGUGGCUCAGCCCCCCUUGCCAUCGGCCUCUCUGUAGCCCUUGGACACCUCCU | Cargo-PIE1- |
| | GGCUAUUGACUACACUGGCUGUGGGAUUAACCCUGCUCGGUCCUUUGGCUCCGCGGUGAUCACACAC | CVB3-AQP1 |
| | AACUUCAGCAACCACUGGAUUUUCUGGGUGGGGCCAUUCAUCGGGGGAGCCCUGGCUGUACUCAUCU | circRNA |
| | ACGACUUCAUCCUGGCCCCACGCAGCAGUGACCUCACAGACCGCGUGAAGGUGUGGACCAGCGGCCA | |
| | GGUGGAGGAGUAUGACCUGGAUGCCGACGACAUCAACUCCAGGGUGGAGAUGAAGCCCAAAUGAUAA | |
| | UUAAAACAGCCUGUGGGUUGAUCCCACCCACAGGCCCAUUGGGCGCUAGCACUCUGGUAUCACGGUA | |
| | CCUUUGUGCGCCUGUUUUAUACCCCCUCCCCCAACUGUAACUUAGAAGUAACACACACCGAUCAACA | |
| | GUCAGCGUGGCACACCAGCCACGUUUUGAUCAAGCACUUCUGUUACCCCGGACUGAGUAUCAAUAGA | |
| | CUGCUCACGCGGUUGAAGGAGAAAGCGUUCGUUAUCCGGCCAACUACUUCGAAAAACCUAGUAACAC | |
| | CGUGGAAGUUGCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCAUGGGACGCUC | |
| | UAAUACAGACAUGGUGCGAAGAGUCUAUUGAGCUAGUUGGUAGUCCUCCGGCCCCUGAAUGCGGCUA | |
| | AUCCUAACUGCGGAGCACACACCCUCAAGCCAGAGGGCAGUGUGUCGUAACGGGCAACUCUGCAGCG | |
| | GAACCGACUACUUUGGGUGUCCGUGUUUCAUUUUAUUCCUAUACUGGCUGCUUAUGGUGACAAUUGA | |
| | GAGAUCGUUACCAUAUAGCUAUUGGAUUGGCCAUCCGGUGACUAAUAGAGCUAUUAUAUAUCCCUUU | |
| | GUUGGGUUUAUACCACUUAGCUUGAAAGAGGUUAAAACAUUACAAUUCAUUGUUAAGUUGAAUACAG | |
| | CAAAGCCACCAUGGCCAGCGAGUUCAAGAAGAAGCUCUUCUGGAGGGCAGUGGUGGCCGAGUUCCUG | |
| | GCCACGACCCUCUUUGUCUUCAUCAGCAUCGGUUCUGCCCUGGGCUUCAAAUACCCGGUGGGGAACA | |
| | ACCAGACGGCGGUCCAGGACAACGUGAAGGUGUCGCUGGCCUUCGGGCUGAGCAUCGCCACGCUGGC | |
| | GCAGAGUGUGGGCCACAUCAGCGGCGCCCACCUCAACCCGGCUGUCACACUGGGGCUGCUGCUCAGC | |
| | UGCCAGAUCAGCAUCUUCCGUGCCCUCAUGUACAUCAUCGCCCAGUGCGUGGGGGCCAUCGUCGCCA | |
| | CCGCCAUCCUCUCUGGCAU | |
| 551 | AGAAGUAACACACACCGAUCAACAGUCAGCGUGGCACACCAGCCACGUUUUGAUCAAGCACUUCUGU | AnaX-GCv3- |
| | UACCCCGGACUGAGUAUCAAUAGACUGCUCACGCGGUUGAAGGAGAAAGCGUUCGUUAUCCGGCCAA | 2 x homo- |
| | CUACUUCGAAAAACCUAGUAACACCGUGGAAGUUGCAGAGUGUUUCGCUCAGCACUACCCCAGUGUA | CLEAN-PIE- |
| | GAUCAGGUCGAUGAGUCACCGCAUUCCCCACGGGCGACCGUGGCGGUGGCUGCGUUGGCGGCCUGCC | IRES- |
| | CAUGGGGAAACCCAUGGGACGCUCUAAUACAGACAUGGUGCGAAGAGUCUAUUGAGCUAGUUGGUAG | rawCVB3-AQP1 |
| | UCCUCCGGCCCCUGAAUGCGGCUAAUCCUAACUGCGGAGCACACACCCUCAAGCCAGAGGGCAGUGU | circRNA |
| | GUCGUAACGGGCAACUCUGCAGCGGAACCGACUACUUUGGGUGUCCGUGUUUCAUUUUAUUCCUAUA | |
| | CUGGCUGCUUAUGGUGACAAUUGAGAGAUCGUUACCAUAUAGCUAUUGGAUUGGCCAUCCGGUGACU | |
| | AAUAGAGCUAUUAUAUAUCCCUUUGUUGGGUUUAUACCACUUAGCUUGAAAGAGGUUAAAACAUUAC | |
| | AAUUCAUUGUUAAGUUGAAUACAGCAAAGCCACCAUGGCCAGCGAGUUCAAGAAGAAGCUCUUCUGG | |
| | AGGGCAGUGGUGGCCGAGUUCCUGGCCACGACCCUCUUUGUCUUCAUCAGCAUCGGUUCUGCCCUGG | |
| | GCUUCAAAUACCCGGUGGGGAACAACCAGACGGCGGUCCAGGACAACGUGAAGGUGUCGCUGGCCUU | |
| | CGGGCUGAGCAUCGCCACGCUGGCGCAGAGUGUGGGCCACAUCAGCGGCGCCCACCUCAACCCGGCU | |
| | GUCACACUGGGGCUGCUGCUCAGCUGCCAGAUCAGCAUCUUCCGUGCCCUCAUGUACAUCAUCGCCC | |
| | AGUGCGUGGGGGCCAUCGUCGCCACCGCCAUCCUCUCAGGCAUCACCUCCUCCCUGACUGGGAACUC | |
| | GCUUGGCCGCAAUGACCUGGCUGAUGGUGUGAACUCGGGCCAGGGCCUGGGCAUCGAGAUCAUCGGG | |
| | ACCCUCCAGCUGGUGCUAUGCGUGCUGGCUACUACCGACCGGAGGCGCCGUGACCUUGGUGGCUCAG | |
| | CCCCCCUUGCCAUCGGCCUCUCUGUAGCCCUUGGACACCUCCUGGCUAUUGACUACACUGGCUGUGG | |
| | GAUUAACCCUGCUCGGUCCUUUGGCUCCGCGGUGAUCACACACAACUUCAGCAACCACUGGAUUUUC | |
| | UGGGUGGGGCCAUUCAUCGGGGGAGCCCUGGCUGUACUCAUCUACGACUUCAUCCUGGCCCCACGCA | |
| | GCAGUGACCUCACAGACCGCGUGAAGGUGUGGACCAGCGGCCAGGUGGAGGAGUAUGACCUGGAUGC | |
| | CGACGACAUCAACUCCAGGGUGGAGAUGAAGCCCAAAUGAUAAUUAAAACAGCCUGUGGGUUGAUCC | |
| | CACCCACAGGCCCAUUGGGCGCUAGCACUCUGGUAUCACGGUACCUUUGUGCGCCUGUUUUAUACCC | |
| | CCUCCCCCAACUGUAACUU | |
| 552 | AAAAUUACAACACACCGAUCAACAGUCAGCGUGGCACACCAGCCACGUUUUGAUCAAGCACUUCUGU | AnaX-GCv3- |
| | UACCCCGGACUGAGUAUCAAUAGACUGCUCACGCGGUUGAAGGAGAAAGCGUUCGUUAUCCGGCCAA | 2 × homo- |
| | CUACUUCGAAAAACCUAGUAACACCGUGGAAGUUGCAGAGUGUUUCGCUCAGCACUACCCCAGUGUA | CLEAN-PIE- |
| | GAUCAGGUCGAUGAGUCACCGCAUUCCCCACGGGCGACCGUGGCGGUGGCUGCGUUGGCGGCCUGCC | IRES- |
| | CAUGGGGAAACCCAUGGGACGCUCUAAUACAGACAUGGUGCGAAGAGUCUAUUGAGCUAGUUGGUAG | mutationCVB3- |
| | UCCUCCGGCCCCUGAAUGCGGCUAAUCCUAACUGCGGAGCACACACCCUCAAGCCAGAGGGCAGUGU | AQP1 |
| | GUCGUAACGGGCAACUCUGCAGCGGAACCGACUACUUUGGGUGUCCGUGUUUCAUUUUAUUCCUAUA | circRNA |
| | CUGGCUGCUUAUGGUGACAAUUGAGAGAUCGUUACCAUAUAGCUAUUGGAUUGGCCAUCCGGUGACU | |
| | AAUAGAGCUAUUAUAUAUCCCUUUGUUGGGUUUAUACCACUUAGCUUGAAAGAGGUUAAAACAUUAC | |
| | AAUUCAUUGUUAAGUUGAAUACAGCAAAGCCACCAUGGCCAGCGAGUUCAAGAAGAAGCUCUUCUGG | |
| | AGGGCAGUGGUGGCCGAGUUCCUGGCCACGACCCUCUUUGUCUUCAUCAGCAUCGGUUCUGCCCUGG | |
| | GCUUCAAAUACCCGGUGGGGAACAACCAGACGGCGGUCCAGGACAACGUGAAGGUGUCGCUGGCCUU | |
| | CGGGCUGAGCAUCGCCACGCUGGCGCAGAGUGUGGGCCACAUCAGCGGCGCCCACCUCAACCCGGCU | |
| | GUCACACUGGGGCUGCUGCUCAGCUGCCAGAUCAGCAUCUUCCGUGCCCUCAUGUACAUCAUCGCCC | |
| | AGUGCGUGGGGGCCAUCGUCGCCACCGCCAUCCUCUCAGGCAUCACCUCCUCCCUGACUGGGAACUC | |
| | GCUUGGCCGCAAUGACCUGGCUGAUGGUGUGAACUCGGGCCAGGGCCUGGGCAUCGAGAUCAUCGGG | |
| | ACCCUCCAGCUGGUGCUAUGCGUGCUGGCUACUACCGACCGGAGGCGCCGUGACCUUGGUGGCUCAG | |
| | CCCCCCUUGCCAUCGGCCUCUCUGUAGCCCUUGGACACCUCCUGGCUAUUGACUACACUGGCUGUGG | |
| | GAUUAACCCUGCUCGGUCCUUUGGCUCCGCGGUGAUCACACACAACUUCAGCAACCACUGGAUUUUC | |
| | UGGGUGGGGCCAUUCAUCGGGGGAGCCCUGGCUGUACUCAUCUACGACUUCAUCCUGGCCCCACGCA | |
| | GCAGUGACCUCACAGACCGCGUGAAGGUGUGGACCAGCGGCCAGGUGGAGGAGUAUGACCUGGAUGC | |
| | CGACGACAUCAACUCCAGGGUGGAGAUGAAGCCCAAAUGAUAAUUAAAACAGCCUGUGGGUUGAUCC | |
| | CACCCACAGGCCCAUUGGGCGCUAGCACUCUGGUAUCACGGUACCUUUGUGCGCCUGUUUUAUACCC | |
| | CCUCCCCCAACUGUAACUU | |
| 553 | AAAACCCCCCCAUUAAAACAGCCUGUGGGUUGAUCCCACCCACAGGCCCAUUGGGCGCUAGCACUCU | AnaX-MEHA1- |
| | GGUAUCACGGUACCUUUGUGCGCCUGUUUUAUACCCCCUCCCCCAACUGUAACUUAGAAGUAACACA | CVB3-IL2 |
| | CACCGAUCAACAGUCAGCGUGGCACACCAGCCACGUUUUGAUCAAGCACUUCUGUUACCCCGGACUG | circRNA |
| | AGUAUCAAUAGACUGCUCACGCGGUUGAAGGAGAAAGCGUUCGUUAUCCGGCCAACUACUUCGAAAA | |
| | ACCUAGUAACACCGUGGAAGUUGCAGAGUGUUUCGCUCAGCACUACCCCAGUGUAGAUCAGGUCGAU | |
| | GAGUCACCGCAUUCCCCACGGGCGACCGUGGCGGUGGCUGCGUUGGCGGCCUGCCCAUGGGGAAACC | |
| | CAUGGGACGCUCUAAUACAGACAUGGUGCGAAGAGUCUAUUGAGCUAGUUGGUAGUCCUCCGGCCCC | |
| | UGAAUGCGGCUAAUCCUAACUGCGGAGCACACACCCUCAAGCCAGAGGGCAGUGUGUCGUAACGGGC | |
| | AACUCUGCAGCGGAACCGACUACUUUGGGUGUCCGUGUUUCAUUUUAUUCCUAUACUGGCUGCUUAU | |
| | GGUGACAAUUGAGAGAUCGUUACCAUAUAGCUAUUGGAUUGGCCAUCCGGUGACUAAUAGAGCUAUU | |
| | AUAUAUCCCUUUGUUGGGUUUAUACCACUUAGCUUGAAAGAGGUUAAAACAUUACAAUUCAUUGUUA | |
| | AGUUGAAUACAGCAAAGCCACCAUGUAUAGAAUGCAGCUGCUGUCUUGCAUCGCCCUGAGCCUGGCC | |
| | CUGGUCACAAACAGCGCUCCUACAAGCUCUAGCACCAAGAAAACCCAGCUGCAGCUCGAGCACCUGC | |
| | UGCUGGACCUGCAGAUGAUCCUGAACGGCAUUAACAACUACAAGAACCCAAAGCUGACCAGAAUGCU | |
| | GACAUUUAAGUUCUACAUGCCUAAGAAGGCCACAGAGCUGAAGCACCUGCAAUGUCUGGAAGAGGAA | |
| | CUGAAGCCCCUGGAAGAGGUGCUUAAUCUGGCUCAGAGCAAGAACUUCCACCUGAGACCUCGGGACC | |
| | UGAUCAGCGACAUCAACGUGAUCGUGCUGGAACUGAAAGGCAGCGAGACAACCUUCAUGUGCGAGUA | |
| | CGCCGAUGAGACCGCCACCAUCGUGGAGUUCCUGAAUCGGUGGAUCACCUUCUGCCAGUCCAUCAUC | |
| | UCCACCCUGACCUGAUAAAGACGGGGGGGGCUU | |
| 554 | AGAAGUAACACACACCGAUCAACAGUCAGCGUGGCACACCAGCCACGUUUUGAUCAAGCACUUCUGU | AnaX-CLEAN- |
| | UACCCCGGACUGAGUAUCAAUAGACUGCUCACGCGGUUGAAGGAGAAAGCGUUCGUUAUCCGGCCAA | PIE-IRES - |
| | CUACUUCGAAAAACCUAGUAACACCGUGGAAGUUGCAGAGUGUUUCGCUCAGCACUACCCCAGUGUA | rawCVB3- |
| | GAUCAGGUCGAUGAGUCACCGCAUUCCCCACGGGCGACCGUGGCGGUGGCUGCGUUGGCGGCCUGCC | 3′, AnaX- |
| | CAUGGGGAAACCCAUGGGACGCUCUAAUACAGACAUGGUGCGAAGAGUCUAUUGAGCUAGUUGGUAG | GCv3-2 × homo- |
| | UCCUCCGGCCCCUGAAUGCGGCUAAUCCUAACUGCGGAGCACACACCCUCAAGCCAGAGGGCAGUGU | CLEAN-PIE- |
| | GUCGUAACGGGCAACUCUGCAGCGGAACCGACUACUUUGGGUGUCCGUGUUUCAUUUUAUUCCUAUA | IRES - |
| | CUGGCUGCUUAUGGUGACAAUUGAGAGAUCGUUACCAUAUAGCUAUUGGAUUGGCCAUCCGGUGACU | rawCVB3-3′ |
| | AAUAGAGCUAUUAUAUAUCCCUUUGUUGGGUUUAUACCACUUAGCUUGAAAGAGGUUAAAACAUUAC | |
| | AAUUCAUUGUUAAGUUGAAUACAGCAAA | |
| 555 | UUAAAACAGCCUGUGGGUUGAUCCCACCCACAGGCCCAUUGGGCGCUAGCACUCUGGUAUCACGGUA | AnaX-CLEAN- |
| | CCUUUGUGCGCCUGUUUUAUACCCCCUCCCCCAACUGUAACUU | PIE-IRES- |
| | | rawCVB3- |
| | | 5′, AnaX- |
| | | CLEAN-PIE- |
| | | IRES- |
| | | rawMutation2- |
| | | CVB3- |
| | | 5′, AnaX- |
| | | GCv3-2 × homo- |
| | | CLEAN-PIE- |
| | | IRES- |
| | | rawCVB3- |
| | | 5′, AnaX- |
| | | GCv3-2 × homo- |
| | | CLEAN-PIE- |
| | | IRES - |
| | | mutationCVB3- |
| | | 5′ |
| 556 | AaAAUUAcaACACACCGAUCAACAGUCAGCGUGGCACACCAGCCACGUUUUGAUCAAGCACUUCUGU | AnaX-CLEAN- |
| | UACCCCGGACUGAGUAUCAAUAGACUGCUCACGCGGUUGAAGGAGAAAGCGUUCGUUAUCCGGCCAA | PIE-IRES- |
| | CUACUUCGAAAAACCUAGUAACACCGUGGAAGUUGCAGAGUGUUUCGCUCAGCACUACCCCAGUGUA | rawMutation2- |
| | GAUCAGGUCGAUGAGUCACCGCAUUCCCCACGGGCGACCGUGGCGGUGGCUGCGUUGGCGGCCUGCC | CVB3- |
| | CAUGGGGAAACCCAUGGGACGCUCUAAUACAGACAUGGUGCGAAGAGUCUAUUGAGCUAGUUGGUAG | 3′, AnaX- |
| | UCCUCCGGCCCCUGAAUGCGGCUAAUCCUAACUGCGGAGCACACACCCUCAAGCCAGAGGGCAGUGU | GCv3-2 × homo- |
| | GUCGUAACGGGCAACUCUGCAGCGGAACCGACUACUUUGGGUGUCCGUGUUUCAUUUUAUUCCUAUA | CLEAN-PIE- |
| | CUGGCUGCUUAUGGUGACAAUUGAGAGAUCGUUACCAUAUAGCUAUUGGAUUGGCCAUCCGGUGACU | IRES- |
| | AAUAGAGCUAUUAUAUAUCCCUUUGUUGGGUUUAUACCACUUAGCUUGAAAGAGGUUAAAACAUUAC | mutationCVB3- |
| | AAUUCAUUGUUAAGUUGAAUACAGCAAA | 3′ |
| 557 | aAAcCUCGCUCAGAGCAAGAACUUCCACCUGAGACCUCGGGACCUGAUCAGCGACAUCAACGUGAUC | AnaX-CLEAN- |
| | GUGCUGGAACUGAAAGGCAGCGAGACAACCUUCAUGUGCGAGUACGCCGAUGAGACCGCCACCAUCG | PIE-Cargo- |
| | UGGAGUUCCUGAAUCGGUGGAUCACCUUCUGCCAGUCCAUCAUCUCCACCCUGACC | PIE1-3′ |
| 558 | AUGUAUAGAAUGCAGCUGCUGUCUUGCAUCGCCCUGAGCCUGGCCCUGGUCACAAACAGCGCUCCUA | AnaX-CLEAN- |
| | CAAGCUCUAGCACCAAGAAAACCCAGCUGCAGCUCGAGCACCUGCUGCUGGACCUGCAGAUGAUCCU | PIE-Cargo- |
| | GAACGGCAUUAACAACUACAAGAACCCAAAGCUGACCAGAAUGCUGACAUUUAAGUUCUACAUGCCU | PIE1-5′ |
| | AAGAAGGCCACAGAGCUGAAGCACCUGCAAUGUCUGGAAGAGGAACUGAAGCCCCUGGAAGAGGUCU | |
| | U | |
| 559 | aAUCUCUGACAUCAACGUGAUCGUGCUGGAACUGAAAGGCAGCGAGACAACCUUCAUGUGCGAGUAC | AnaX-CLEAN- |
| | GCCGAUGAGACCGCCACCAUCGUGGAGUUCCUGAAUCGGUGGAUCACCUUCUGCCAGUCCAUCAUCU | PIE-Cargo- |
| | CCACCCUGACC | PIE2-3′ |
| 560 | AUGUAUAGAAUGCAGCUGCUGUCUUGCAUCGCCCUGAGCCUGGCCCUGGUCACAAACAGC | AnaX-CLEAN- |
| | | PIE-Cargo- |
| | | PIE2-5′ |
| 561 | aACUGGaAACUCGCUUGGCCGCAAUGACCUGGCUGAUGGUGUGAACUCGGGCCAGGGCCUGGGCAUC | AnaX-GCv3- |
| | GAGAUCAUCGGGACCCUCCAGCUGGUGCUAUGCGUGCUGGCUACUACCGACCGGAGGCGCCGUGACC | 2 × homo- |
| | UUGGUGGCUCAGCCCCCCUUGCCAUCGGCCUCUCUGUAGCCCUUGGACACCUCCUGGCUAUUGACUA | CLEAN-PIE- |
| | CACUGGCUGUGGGAUUAACCCUGCUCGGUCCUUUGGCUCCGCGGUGAUCACACACAACUUCAGCAAC | Cargo-PIE1- |
| | CACUGGAUUUUCUGGGUGGGGCCAUUCAUCGGGGGAGCCCUGGCUGUACUCAUCUACGACUUCAUCC | 3′ |
| | UGGCCCCACGCAGCAGUGACCUCACAGACCGCGUGAAGGUGUGGACCAGCGGCCAGGUGGAGGAGUA | |
| | UGACCUGGAUGCCGACGACAUCAACUCCAGGGUGGAGAUGAAGCCCAAAUGAUAA | |
| 562 | GCCACCAUGGCCAGCGAGUUCAAGAAGAAGCUCUUCUGGAGGGCAGUGGUGGCCGAGUUCCUGGCCA | AnaX-GCv3- |
| | CGACCCUCUUUGUCUUCAUCAGCAUCGGUUCUGCCCUGGGCUUCAAAUACCCGGUGGGGAACAACCA | 2 × homo- |
| | GACGGCGGUCCAGGACAACGUGAAGGUGUCGCUGGCCUUCGGGCUGAGCAUCGCCACGCUGGCGCAG | CLEAN-PIE- |
| | AGUGUGGGCCACAUCAGCGGCGCCCACCUCAACCCGGCUGUCACACUGGGGCUGCUGCUCAGCUGCC | Cargo-PIE1- |
| | AGAUCAGCAUCUUCCGUGCCCUCAUGUACAUCAUCGCCCAGUGCGUGGGGGCCAUCGUCGCCACCGC | |
| | CAUCCUCUCAGGCAUCACCUCCagCUU | |
| 563 | aACCagCUCCCUGACUGGGAACUCGCUUGGCCGCAAUGACCUGGCUGAUGGUGUGAACUCGGGCCAG | Twort-PIE-6- |
| | GGCCUGGGCAUCGAGAUCAUCGGGACCCUCCAGCUGGUGCUAUGCGUGCUGGCUACUACCGACCGGA | CLEAN-PIE- |
| | GGCGCCGUGACCUUGGUGGCUCAGCCCCCCUUGCCAUCGGCCUCUCUGUAGCCCUUGGACACCUCCU | Cargo-PIE1- |
| | GGCUAUUGACUACACUGGCUGUGGGAUUAACCCUGCUCGGUCCUUUGGCUCCGCGGUGAUCACACAC | 3′ |
| | AACUUCAGCAACCACUGGAUUUUCUGGGUGGGGCCAUUCAUCGGGGGAGCCCUGGCUGUACUCAUCU | |
| | ACGACUUCAUCCUGGCCCCACGCAGCAGUGACCUCACAGACCGCGUGAAGGUGUGGACCAGCGGCCA | |
| | GGUGGAGGAGUAUGACCUGGAUGCCGACGACAUCAACUCCAGGGUGGAGAUGAAGCCCAAAUGAUAA | |
| 564 | GCCACCAUGGCCAGCGAGUUCAAGAAGAAGCUCUUCUGGAGGGCAGUGGUGGCCGAGUUCCUGGCCA | Twort-PIE-6- |
| | CGACCCUCUUUGUCUUCAUCAGCAUCGGUUCUGCCCUGGGCUUCAAAUACCCGGUGGGGAACAACCA | CLEAN-PIE- |
| | GACGGCGGUCCAGGACAACGUGAAGGUGUCGCUGGCCUUCGGGCUGAGCAUCGCCACGCUGGCGCAG | Cargo-PIE1- |
| | AGUGUGGGCCACAUCAGCGGCGCCCACCUCAACCCGGCUGUCACACUGGGGCUGCUGCUCAGCUGCC | 5′ |
| | AGAUCAGCAUCUUCCGUGCCCUCAUGUACAUCAUCGCCCAGUGCGUGGGGGCCAUCGUCGCCACCGC | |
| | CAUCCUCUCUGGCAU | |
| 565 | GGGAGACCCUCGACCGUCGAUUGUCCACUGGUCAACAAUAGAUGACUUACAACUAAUCGGAAGGUGC | Anabaena |
| | AGAGACUCGACGGGAGCUACCCUAACGUCAAGACGAGGGUAAAGAGAGAGUCCAAUUCUCAAAGCCA | pre-tRNA-Leu |
| | AUAGGCAGUAGCGAAAGCUGCAAGAGAAUGAAAAUCCGUUGACCGGCAGCCAACUCUGCACAAAAUG | gene GROUP I |
| | UCCAUGAUGGGGCAUCGGGUCCGCAUUCUCCCAUGGUCUACCUUUCGCUUGAAUCUUAGUGUGACAA | INTRON |
| | CUCCGUACAAUGCAGACUUUGACGGGGAUGAGAUGAACUUGCACCUGCCACAGUCUCUGGAGACGCG | POLR2A |
| | AGCAGAGAUCCAGGAGCUGGCCAUGGUUCCUCGCAUGAUUGUCACCCCCCAGAGCAAUCGGCCUGUC | precursor |
| | AUGGGUAUUGUGCAGGACACACUCACAGCAGUGCGCAAAUUCACCAAGAGAGACGUCUUCCUGGAGC | |
| | GGGUGGAACGGCACAUGUGUGAUGGGGACAUUGUUAUCUUCAAAGACGCUACGGACUUAAAUAAUUG | |
| | AGCCUUAAAGAAGAAAUUCUUUAAGUGGAUGCUCUCAAACUCAGGGAAACCUAAAUCUAGUUAUAGA | |
| | CAAGGCAAUCCUGAGCCAAGCCGAAGUAGUAAUUAGUAAGACCAGUGGACAAUCGACGGAUAACAGC | |
| | AUAUCUAG | |
| 566 | GGGAGACCCUCGACCGUCGAUUGUCCACUGGUCAACCACGCAAGGGAUGGUGUCAAAUUCGGCGAAA | Azoarcus |
| | CCUAAGCGCCCGCCCGGGCGUAUGGCAACGCCGAGCCAAGCUUCGGCGCCUGCGCCGAUGAAGGUGU | olearius |
| | AGAGACUAGACGGCACCCACCUAAGGCAAACGCUAUGGUGAAGGCAUAGUCCAGGGAGUGGCGAAAG | BH72 GROUP I |
| | UCACACAAACCGGaaUccgUUggUgcUgCCGGCAGCCAACUCUGCACAAAAUGUCCAUGAUGGGGCA | INTRON-PIE-2 |
| | UCGGGUCCGCAUUCUCCCAUGGUCUACCUUUCGCUUGAAUCUUAGUGUGACAACUCCGUACAAUGCA | POLR2A |
| | GACUUUGACGGGGAUGAGAUGAACUUGCACCUGCCACAGUCUCUGGAGACGCGAGCAGAGAUCCAGG | precursor |
| | AGCUGGCCAUGGUUCCUCGCAUGAUUGUCACCCCCCAGAGCAAUCGGCCUGUCAUGGGUAUUGUGCA | |
| | GGACACACUCACAGCAGUGCGCAAAUUCACCAAGAGAGACGUCUUCCUGGAGCGGGUGGAACGGCAC | |
| | AUGUGUGAUGGGGACAUUGUUAUCUUCAAgagcagcggacUcaUAUUUCGAUGUGCCUUGCGCCGGG | |
| | AGACCAGUGGACAAUCGACGGAUAACAGCAUAUCUAG | |
| 567 | GGGAGACCCUCGACCGUCGAUUGUCCACUGGUCACGCCGAGCCAAGCUUCGGCGCCUGCGCCGAUGA | Azoarcus |
| | AGGUGUAGAGACUAGACGGCACCCACCUAAGGCAAACGCUAUGGUGAAGGCAUAGUCCAGGGAGUGG | olearius |
| | CGAAAGUCACACAAACCGGaaUccgUUggUgcUgCCGGCAGCCAACUCUGCACAAAAUGUCCAUGAU | BH72 GROUP I |
| | GGGGCAUCGGGUCCGCAUUCUCCCAUGGUCUACCUUUCGCUUGAAUCUUAGUGUGACAACUCCGUAC | INTRON-PIE- |
| | AAUGCAGACUUUGACGGGGAUGAGAUGAACUUGCACCUGCCACAGUCUCUGGAGACGCGAGCAGAGA | 4-5 POLR2A |
| | UCCAGGAGCUGGCCAUGGUUCCUCGCAUGAUUGUCACCCCCCAGAGCAAUCGGCCUGUCAUGGGUAU | precursor |
| | UGUGCAGGACACACUCACAGCAGUGCGCAAAUUCACCAAGAGAGACGUCUUCCUGGAGCGGGUGGAA | |
| | CGGCACAUGUGUGAUGGGGACAUUGUUAUCUUCAAgagcagcggacUcaUAUUUCGAUGUGCCUUGC | |
| | GCCGGGAAACCACGCAAGGGAUGGUGUCAAAUUCGGCGAAACCUAAGCGCCCGCCCGGGCGUAUGGC | |
| | AGACCAGUGGACAAUCGACGGAUAACAGCAUAUCUAG | |
| 568 | GGGAGACCCUCGACCGUCGAUUGUCCACUGGUCCCGGGCGUAUGGCAACGCCGAGCCAAGCUUCGGC | Azoarcus |
| | GCCUGCGCCGAUGAAGGUGUAGAGACUAGACGGCACCCACCUAAGGCAAACGCUAUGGUGAAGGCAU | olearius |
| | AGUCCAGGGAGUGGCGAAAGUCACACAAACCGGaaUccgUUggUgcUgCCGGCAGCCAACUCUGCAC | BH72 GROUP I |
| | AAAAUGUCCAUGAUGGGGCAUCGGGUCCGCAUUCUCCCAUGGUCUACCUUUCGCUUGAAUCUUAGUG | INTRON-PIE-5 |
| | UGACAACUCCGUACAAUGCAGACUUUGACGGGGAUGAGAUGAACUUGCACCUGCCACAGUCUCUGGA | POLR2A |
| | GACGCGAGCAGAGAUCCAGGAGCUGGCCAUGGUUCCUCGCAUGAUUGUCACCCCCCAGAGCAAUCGG | precursor |
| | CCUGUCAUGGGUAUUGUGCAGGACACACUCACAGCAGUGCGCAAAUUCACCAAGAGAGACGUCUUCC | |
| | UGGAGCGGGUGGAACGGCACAUGUGUGAUGGGGACAUUGUUAUCUUCAAgagcagcggacUcaUAUU | |
| | UCGAUGUGCCUUGCGCCGGGAAACCACGCAAGGGAUGGUGUCAAAUUCGGCGAAACCUAAGCGCCCG | |
| | CGACCAGUGGACAAUCGACGGAUAACAGCAUAUCUAG | |
| 569 | GGGAGACCCUCGACCGUCGAUUGUCCACUGGUCUGCGCCGAUGAAGGUGUAGAGACUAGACGGCACC | Azoarcus |
| | CACCUAAGGCAAACGCUAUGGUGAAGGCAUAGUCCAGGGAGUGGCGAAAGUCACACAAACCGGaaUc | olearius |
| | cgUUggUgcUgCCGGCAGCCAACUCUGCACAAAAUGUCCAUGAUGGGGCAUCGGGUCCGCAUUCUCC | BH72 GROUP I |
| | CAUGGUCUACCUUUCGCUUGAAUCUUAGUGUGACAACUCCGUACAAUGCAGACUUUGACGGGGAUGA | INTRON-PIE-6 |
| | GAUGAACUUGCACCUGCCACAGUCUCUGGAGACGCGAGCAGAGAUCCAGGAGCUGGCCAUGGUUCCU | POLR2A |
| | CGCAUGAUUGUCACCCCCCAGAGCAAUCGGCCUGUCAUGGGUAUUGUGCAGGACACACUCACAGCAG | precursor |
| | UGCGCAAAUUCACCAAGAGAGACGUCUUCCUGGAGCGGGUGGAACGGCACAUGUGUGAUGGGGACAU | |
| | UGUUAUCUUCAAgagcagcggacUcaUAUUUCGAUGUGCCUUGCGCCGGGAAACCACGCAAGGGAUG | |
| | GUGUCAAAUUCGGCGAAACCUAAGCGCCCGCCCGGGCGUAUGGCAACGCCGAGCCAAGCUUCGGCGC | |
| | CGACCAGUGGACAAUCGACGGAUAACAGCAUAUCUAG | |
| 570 | GGGAGACCCUCGACCGUCGAUUGUCCACUGGUCACGCUAUGGUGAAGGCAUAGUCCAGGGAGUGGCG | Azoarcus |
| | AAAGUCACACAAACCGGaaUccgUUggUgcUgCCGGCAGCCAACUCUGCACAAAAUGUCCAUGAUGG | olearius |
| | GGCAUCGGGUCCGCAUUCUCCCAUGGUCUACCUUUCGCUUGAAUCUUAGUGUGACAACUCCGUACAA | BH72 GROUP I |
| | UGCAGACUUUGACGGGGAUGAGAUGAACUUGCACCUGCCACAGUCUCUGGAGACGCGAGCAGAGAUC | INTRON-PIE-8 |
| | CAGGAGCUGGCCAUGGUUCCUCGCAUGAUUGUCACCCCCCAGAGCAAUCGGCCUGUCAUGGGUAUUG | POLR2A |
| | UGCAGGACACACUCACAGCAGUGCGCAAAUUCACCAAGAGAGACGUCUUCCUGGAGCGGGUGGAACG | precursor |
| | GCACAUGUGUGAUGGGGACAUUGUUAUCUUCAAgagcagcggacUcaUAUUUCGAUGUGCCUUGCGC | |
| | CGGGAAACCACGCAAGGGAUGGUGUCAAAUUCGGCGAAACCUAAGCGCCCGCCCGGGCGUAUGGCAA | |
| | CGCCGAGCCAAGCUUCGGCGCCUGCGCCGAUGAAGGUGUAGAGACUAGACGGCACCCACCUAAGGCA | |
| | AGACCAGUGGACAAUCGACGGAUAACAGCAUAUCUAG | |
| 571 | GGGAGACCCUCGACCGUCGAUUGUCCACUGGUCAAACCGGaaUccgUUggUgcUgCCGGCAGCCAAC | Azoarcus |
| | UCUGCACAAAAUGUCCAUGAUGGGGCAUCGGGUCCGCAUUCUCCCAUGGUCUACCUUUCGCUUGAAU | olearius |
| | CUUAGUGUGACAACUCCGUACAAUGCAGACUUUGACGGGGAUGAGAUGAACUUGCACCUGCCACAGU | BH72 GROUP I |
| | CUCUGGAGACGCGAGCAGAGAUCCAGGAGCUGGCCAUGGUUCCUCGCAUGAUUGUCACCCCCCAGAG | INTRON-PIE-9 |
| | CAAUCGGCCUGUCAUGGGUAUUGUGCAGGACACACUCACAGCAGUGCGCAAAUUCACCAAGAGAGAC | POLR2A |
| | GUCUUCCUGGAGCGGGUGGAACGGCACAUGUGUGAUGGGGACAUUGUUAUCUUCAAgagcagcggac | precursor |
| | UcaUAUUUCGAUGUGCCUUGCGCCGGGAAACCACGCAAGGGAUGGUGUCAAAUUCGGCGAAACCUAA | |
| | GCGCCCGCCCGGGCGUAUGGCAACGCCGAGCCAAGCUUCGGCGCCUGCGCCGAUGAAGGUGUAGAGA | |
| | CUAGACGGCACCCACCUAAGGCAAACGCUAUGGUGAAGGCAUAGUCCAGGGAGUGGCGAAAGUCACA | |
| | CGACCAGUGGACAAUCGACGGAUAACAGCAUAUCUAG | |
| 572 | GGGAGACCCUCGACCGUCGAUUGUCCACUGGUCAAUGUAUAUCGAAAAAUCCUCUAAUUCAGGGAAC | Staphylococcus |
| | ACCUAAACAAACUAAGAUGUAGGCAAUCCUGAGCUAAGCUCUUAGUAAUAAGAGAAAGUGCAACGAC | phage |
| | UAUUCCGAUAGGAAGUAGGGUCAAGUGACUCGAAAUGGGGAUUACCCUUCUAGGGUAGUGAUAUAGU | Twort GROUP |
| | CUGAACAUAUAUGGAAACAUAUAGAAGGAUAGGAGUAACGAACCUAUUCGUAACAUAAUUGaacUUU | I INTRON- |
| | UagUUaUCCGGCAGCCAACUCUGCACAAAAUGUCCAUGAUGGGGCAUCGGGUCCGCAUUCUCCCAUG | PIE-2 POLR2A |
| | GUCUACCUUUCGCUUGAAUCUUAGUGUGACAACUCCGUACAAUGCAGACUUUGACGGGGAUGAGAUG | precursor |
| | AACUUGCACCUGCCACAGUCUCUGGAGACGCGAGCAGAGAUCCAGGAGCUGGCCAUGGUUCCUCGCA | |
| | UGAUUGUCACCCCCCAGAGCAAUCGGCCUGUCAUGGGUAUUGUGCAGGACACACUCACAGCAGUGCG | |
| | CAAAUUCACCAAGAGAGACGUCUUCCUGGAGCGGGUGGAACGGCACAUGUGUGAUGGGGACAUUGUU | |
| | AUCUUCAAacUacUgaaagcaUAAAUAAUUGUGCCUUUAUACAGUGACCAGUGGACAAUCGACGGAU | |
| | AACAGCAUAUCUAG | |
| 573 | GGGAGACCCUCGACCGUCGAUUGUCCACUGGUCAUCCUGAGCUAAGCUCUUAGUAAUAAGAGAAAGU | Staphylococcus |
| | GCAACGACUAUUCCGAUAGGAAGUAGGGUCAAGUGACUCGAAAUGGGGAUUACCCUUCUAGGGUAGU | phage |
| | GAUAUAGUCUGAACAUAUAUGGAAACAUAUAGAAGGAUAGGAGUAACGAACCUAUUCGUAACAUAAU | Twort GROUP |
| | UGaacUUUUagUUaUCCGGCAGCCAACUCUGCACAAAAUGUCCAUGAUGGGGCAUCGGGUCCGCAUU | I INTRON- |
| | CUCCCAUGGUCUACCUUUCGCUUGAAUCUUAGUGUGACAACUCCGUACAAUGCAGACUUUGACGGGG | PIE-4-5 |
| | AUGAGAUGAACUUGCACCUGCCACAGUCUCUGGAGACGCGAGCAGAGAUCCAGGAGCUGGCCAUGGU | POLR2A |
| | UCCUCGCAUGAUUGUCACCCCCCAGAGCAAUCGGCCUGUCAUGGGUAUUGUGCAGGACACACUCACA | precursor |
| | GCAGUGCGCAAAUUCACCAAGAGAGACGUCUUCCUGGAGCGGGUGGAACGGCACAUGUGUGAUGGGG | |
| | ACAUUGUUAUCUUCAAacUacUgaaagcaUAAAUAAUUGUGCCUUUAUACAGUAAUGUAUAUCGAAA | |
| | AAUCCUCUAAUUCAGGGAACACCUAAACAAACUAAGAUGUAGGCAGACCAGUGGACAAUCGACGGAU | |
| | AACAGCAUAUCUAG | |
| 574 | GGGAGACCCUCGACCGUCGAUUGUCCACUGGUCAAGAUGUAGGCAAUCCUGAGCUAAGCUCUUAGUA | Staphylococcus |
| | AUAAGAGAAAGUGCAACGACUAUUCCGAUAGGAAGUAGGGUCAAGUGACUCGAAAUGGGGAUUACCC | phage |
| | UUCUAGGGUAGUGAUAUAGUCUGAACAUAUAUGGAAACAUAUAGAAGGAUAGGAGUAACGAACCUAU | Twort GROUP |
| | UCGUAACAUAAUUGaacUUUUagUUaUCCGGCAGCCAACUCUGCACAAAAUGUCCAUGAUGGGGCAU | I INTRON- |
| | CGGGUCCGCAUUCUCCCAUGGUCUACCUUUCGCUUGAAUCUUAGUGUGACAACUCCGUACAAUGCAG | PIE-5 POLR2A |
| | ACUUUGACGGGGAUGAGAUGAACUUGCACCUGCCACAGUCUCUGGAGACGCGAGCAGAGAUCCAGGA | precursor |
| | GCUGGCCAUGGUUCCUCGCAUGAUUGUCACCCCCCAGAGCAAUCGGCCUGUCAUGGGUAUUGUGCAG | |
| | GACACACUCACAGCAGUGCGCAAAUUCACCAAGAGAGACGUCUUCCUGGAGCGGGUGGAACGGCACA | |
| | UGUGUGAUGGGGACAUUGUUAUCUUCAAacUacUgaaagcaUAAAUAAUUGUGCCUUUAUACAGUAA | |
| | UGUAUAUCGAAAAAUCCUCUAAUUCAGGGAACACCUAAACAAACUGACCAGUGGACAAUCGACGGAU | |
| | AACAGCAUAUCUAG | |
| 575 | GGGAGACCCUCGACCGUCGAUUGUCCACUGGUCAAUAAGAGAAAGUGCAACGACUAUUCCGAUAGGA | Staphylococcus |
| | AGUAGGGUCAAGUGACUCGAAAUGGGGAUUACCCUUCUAGGGUAGUGAUAUAGUCUGAACAUAUAUG | phage |
| | GAAACAUAUAGAAGGAUAGGAGUAACGAACCUAUUCGUAACAUAAUUGaacUUUUagUUaUCCGGCA | Twort GROUP |
| | GCCAACUCUGCACAAAAUGUCCAUGAUGGGGCAUCGGGUCCGCAUUCUCCCAUGGUCUACCUUUCGC | I INTRON- |
| | UUGAAUCUUAGUGUGACAACUCCGUACAAUGCAGACUUUGACGGGGAUGAGAUGAACUUGCACCUGC | PIE-6 POLR2A |
| | CACAGUCUCUGGAGACGCGAGCAGAGAUCCAGGAGCUGGCCAUGGUUCCUCGCAUGAUUGUCACCCC | precursor |
| | CCAGAGCAAUCGGCCUGUCAUGGGUAUUGUGCAGGACACACUCACAGCAGUGCGCAAAUUCACCAAG | |
| | AGAGACGUCUUCCUGGAGCGGGUGGAACGGCACAUGUGUGAUGGGGACAUUGUUAUCUUCAAacUac | |
| | UgaaagcaUAAAUAAUUGUGCCUUUAUACAGUAAUGUAUAUCGAAAAAUCCUCUAAUUCAGGGAACA | |
| | CCUAAACAAACUAAGAUGUAGGCAAUCCUGAGCUAAGCUCUUAGUGACCAGUGGACAAUCGACGGAU | |
| | AACAGCAUAUCUAG | |
| 576 | GGGAGACCCUCGACCGUCGAUUGUCCACUGGUCUAGGAAGUAGGGUCAAGUGACUCGAAAUGGGGAU | Staphylococcus |
| | UACCCUUCUAGGGUAGUGAUAUAGUCUGAACAUAUAUGGAAACAUAUAGAAGGAUAGGAGUAACGAA | phage |
| | CCUAUUCGUAACAUAAUUGaacUUUUagUUaUCCGGCAGCCAACUCUGCACAAAAUGUCCAUGAUGG | Twort GROUP |
| | GGCAUCGGGUCCGCAUUCUCCCAUGGUCUACCUUUCGCUUGAAUCUUAGUGUGACAACUCCGUACAA | I INTRON- |
| | UGCAGACUUUGACGGGGAUGAGAUGAACUUGCACCUGCCACAGUCUCUGGAGACGCGAGCAGAGAUC | PIE-7 POLR2A |
| | CAGGAGCUGGCCAUGGUUCCUCGCAUGAUUGUCACCCCCCAGAGCAAUCGGCCUGUCAUGGGUAUUG | precursor |
| | UGCAGGACACACUCACAGCAGUGCGCAAAUUCACCAAGAGAGACGUCUUCCUGGAGCGGGUGGAACG | |
| | GCACAUGUGUGAUGGGGACAUUGUUAUCUUCAAacUacUgaaagcaUAAAUAAUUGUGCCUUUAUAC | |
| | AGUAAUGUAUAUCGAAAAAUCCUCUAAUUCAGGGAACACCUAAACAAACUAAGAUGUAGGCAAUCCU | |
| | GAGCUAAGCUCUUAGUAAUAAGAGAAAGUGCAACGACUAUUCCGAGACCAGUGGACAAUCGACGGAU | |
| | AACAGCAUAUCUAG | |
| 577 | GGGAGACCCUCGACCGUCGAUUGUCCACUGGUCCUAGGGUAGUGAUAUAGUCUGAACAUAUAUGGAA | Staphylococcus |
| | ACAUAUAGAAGGAUAGGAGUAACGAACCUAUUCGUAACAUAAUUGaacUUUUagUUaUCCGGCAGCC | phage |
| | AACUCUGCACAAAAUGUCCAUGAUGGGGCAUCGGGUCCGCAUUCUCCCAUGGUCUACCUUUCGCUUG | Twort GROUP |
| | AAUCUUAGUGUGACAACUCCGUACAAUGCAGACUUUGACGGGGAUGAGAUGAACUUGCACCUGCCAC | I INTRON- |
| | AGUCUCUGGAGACGCGAGCAGAGAUCCAGGAGCUGGCCAUGGUUCCUCGCAUGAUUGUCACCCCCCA | PIE-8 POLR2A |
| | GAGCAAUCGGCCUGUCAUGGGUAUUGUGCAGGACACACUCACAGCAGUGCGCAAAUUCACCAAGAGA | precursor |
| | GACGUCUUCCUGGAGCGGGUGGAACGGCACAUGUGUGAUGGGGACAUUGUUAUCUUCAAacUacUga | |
| | aagcaUAAAUAAUUGUGCCUUUAUACAGUAAUGUAUAUCGAAAAAUCCUCUAAUUCAGGGAACACCU | |
| | AAACAAACUAAGAUGUAGGCAAUCCUGAGCUAAGCUCUUAGUAAUAAGAGAAAGUGCAACGACUAUU | |
| | CCGAUAGGAAGUAGGGUCAAGUGACUCGAAAUGGGGAUUACCCUUGACCAGUGGACAAUCGACGGAU | |
| | AACAGCAUAUCUAG | |
| 578 | GGGAGACCCUCGACCGUCGAUUGUCCACUGGUCAACAUAUAGAAGGAUAGGAGUAACGAACCUAUUC | Staphylococcus |
| | GUAACAUAAUUGaacUUUUagUUaUCCGGCAGCCAACUCUGCACAAAAUGUCCAUGAUGGGGCAUCG | phage |
| | GGUCCGCAUUCUCCCAUGGUCUACCUUUCGCUUGAAUCUUAGUGUGACAACUCCGUACAAUGCAGAC | Twort GROUP |
| | UUUGACGGGGAUGAGAUGAACUUGCACCUGCCACAGUCUCUGGAGACGCGAGCAGAGAUCCAGGAGC | I INTRON- |
| | UGGCCAUGGUUCCUCGCAUGAUUGUCACCCCCCAGAGCAAUCGGCCUGUCAUGGGUAUUGUGCAGGA | PIE-9 POLR2A |
| | CACACUCACAGCAGUGCGCAAAUUCACCAAGAGAGACGUCUUCCUGGAGCGGGUGGAACGGCACAUG | precursor |
| | UGUGAUGGGGACAUUGUUAUCUUCAAacUacUgaaagcaUAAAUAAUUGUGCCUUUAUACAGUAAUG | |
| | UAUAUCGAAAAAUCCUCUAAUUCAGGGAACACCUAAACAAACUAAGAUGUAGGCAAUCCUGAGCUAA | |
| | GCUCUUAGUAAUAAGAGAAAGUGCAACGACUAUUCCGAUAGGAAGUAGGGUCAAGUGACUCGAAAUG | |
| | GGGAUUACCCUUCUAGGGUAGUGAUAUAGUCUGAACAUAUAUGGAGACCAGUGGACAAUCGACGGAU | |
| | AACAGCAUAUCUAG | |
| 579 | GGGAGACCCUCGACCGUCGAUUGUCCACUGGUCUAACGAACCUAUUCGUAACAUAAUUGaacUUUUa | Staphylococcus |
| | gUUaUCCGGCAGCCAACUCUGCACAAAAUGUCCAUGAUGGGGCAUCGGGUCCGCAUUCUCCCAUGGU | phage |
| | CUACCUUUCGCUUGAAUCUUAGUGUGACAACUCCGUACAAUGCAGACUUUGACGGGGAUGAGAUGAA | Twort GROUP |
| | CUUGCACCUGCCACAGUCUCUGGAGACGCGAGCAGAGAUCCAGGAGCUGGCCAUGGUUCCUCGCAUG | I INTRON- |
| | AUUGUCACCCCCCAGAGCAAUCGGCCUGUCAUGGGUAUUGUGCAGGACACACUCACAGCAGUGCGCA | PIE-9.1 |
| | AAUUCACCAAGAGAGACGUCUUCCUGGAGCGGGUGGAACGGCACAUGUGUGAUGGGGACAUUGUUAU | POLR2A |
| | CUUCAAacUacUgaaagcaUAAAUAAUUGUGCCUUUAUACAGUAAUGUAUAUCGAAAAAUCCUCUAA | precursor |
| | UUCAGGGAACACCUAAACAAACUAAGAUGUAGGCAAUCCUGAGCUAAGCUCUUAGUAAUAAGAGAAA | |
| | GUGCAACGACUAUUCCGAUAGGAAGUAGGGUCAAGUGACUCGAAAUGGGGAUUACCCUUCUAGGGUA | |
| | GUGAUAUAGUCUGAACAUAUAUGGAAACAUAUAGAAGGAUAGGAGGACCAGUGGACAAUCGACGGAU | |
| | AACAGCAUAUCUAG | |
| 580 | GGGAGACCCUCGACCGUCGAUUGUCCACUGGUCGCGCCCGAGUACCAGGACCGUCGGGCCCCCGAGA | Scytalidium |
| | AGGGGGGGCCUAGGAUGCGGCAAGACGACCCGGUUCGGGGAACGCCAGCGUGCGCUGGCCGAUCCCG | dimidiatum |
| | AGGCGAGGUGCCGUAGCGGGCACCCGUCGUAACGCGCGGUAGGGCGUCGGUCCCCCCUCCCACGGCG | GROUP I |
| | GAGGGGAGGCUUAAGGUACGUGCUAAACCCCCAGCGAUGGGGCCUGUAGGAAAAGCCCUGGUACGGC | INTRON-PIE-2 |
| | GAAGCCUACGGGGACCGCCCGAUGGCGGUCGGAUGCCGGCGGGCCACGGAGCCCCCGGCGUCGAUUG | POLR2A |
| | CUGUUACCGGCAGCCAACUCUGCACAAAAUGUCCAUGAUGGGGCAUCGGGUCCGCAUUCUCCCAUGG | precursor |
| | UCUACCUUUCGCUUGAAUCUUAGUGUGACAACUCCGUACAAUGCAGACUUUGACGGGGAUGAGAUGA | |
| | ACUUGCACCUGCCACAGUCUCUGGAGACGCGAGCAGAGAUCCAGGAGCUGGCCAUGGUUCCUCGCAU | |
| | GAUUGUCACCCCCCAGAGCAAUCGGCCUGUCAUGGGUAUUGUGCAGGACACACUCACAGCAGUGCGC | |
| | AAAUUCACCAAGAGAGACGUCUUCCUGGAGCGGGUGGAACGGCACAUGUGUGAUGGGGACAUUGUUA | |
| | UCUUCAAUAACAGGUUGACGACAACAGGCCUGUGACAGCGGGGCUUCGAACUUUCCGUGUUUUUUUU | |
| | UCUUCCGCUAGUCGAUCAUCCGUGACGGCCGGGCAAGACCAGUGGACAAUCGACGGAUAACAGCAUA | |
| | UCUAG | |
| 581 | GGGAGACCCUCGACCGUCGAUUGUCCACUGGUCGCGCUGGCCGAUCCCGAGGCGAGGUGCCGUAGCG | Scytalidium |
| | GGCACCCGUCGUAACGCGCGGUAGGGCGUCGGUCCCCCCUCCCACGGCGGAGGGGAGGCUUAAGGUA | dimidiatum |
| | CGUGCUAAACCCCCAGCGAUGGGGCCUGUAGGAAAAGCCCUGGUACGGCGAAGCCUACGGGGACCGC | GROUP I |
| | CCGAUGGCGGUCGGAUGCCGGCGGGCCACGGAGCCCCCGGCGUCGAUUGcUgUUaCCGGCAGCCAAC | INTRON-PIE-5 |
| | UCUGCACAAAAUGUCCAUGAUGGGGCAUCGGGUCCGCAUUCUCCCAUGGUCUACCUUUCGCUUGAAU | POLR2A |
| | CUUAGUGUGACAACUCCGUACAAUGCAGACUUUGACGGGGAUGAGAUGAACUUGCACCUGCCACAGU | precursor |
| | CUCUGGAGACGCGAGCAGAGAUCCAGGAGCUGGCCAUGGUUCCUCGCAUGAUUGUCACCCCCCAGAG | |
| | CAAUCGGCCUGUCAUGGGUAUUGUGCAGGACACACUCACAGCAGUGCGCAAAUUCACCAAGAGAGAC | |
| | GUCUUCCUGGAGCGGGUGGAACGGCACAUGUGUGAUGGGGACAUUGUUAUCUUCAAUaacaggUUGA | |
| | CGACAACAGGCCUGUGACAGCGGGGCUUCGAACUUUCCGUGUUUUUUUUUCUUCCGCUAGUCGAUCA | |
| | UCCGUGACGGCCGGGCAAGCGCCCGAGUACCAGGACCGUCGGGCCCCCGAGAAGGGGGGGCCUAGGA | |
| | UGCGGCAAGACGACCCGGUUCGGGGAACGCCAGCGUGACCAGUGGACAAUCGACGGAUAACAGCAUA | |
| | UCUAG | |
| 582 | GGGAGACCCUCGACCGUCGAUUGUCCACUGGUCGCGGGCACCCGUCGUAACGCGCGGUAGGGCGUCG | Scytalidium |
| | GUCCCCCCUCCCACGGCGGAGGGGAGGCUUAAGGUACGUGCUAAACCCCCAGCGAUGGGGCCUGUAG | dimidiatum |
| | GAAAAGCCCUGGUACGGCGAAGCCUACGGGGACCGCCCGAUGGCGGUCGGAUGCCGGCGGGCCACGG | GROUP I |
| | AGCCCCCGGCGUCGAUUGcUgUUaCCGGCAGCCAACUCUGCACAAAAUGUCCAUGAUGGGGCAUCGG | INTRON-PIE-6 |
| | GUCCGCAUUCUCCCAUGGUCUACCUUUCGCUUGAAUCUUAGUGUGACAACUCCGUACAAUGCAGACU | POLR2A |
| | UUGACGGGGAUGAGAUGAACUUGCACCUGCCACAGUCUCUGGAGACGCGAGCAGAGAUCCAGGAGCU | precursor |
| | GGCCAUGGUUCCUCGCAUGAUUGUCACCCCCCAGAGCAAUCGGCCUGUCAUGGGUAUUGUGCAGGAC | |
| | ACACUCACAGCAGUGCGCAAAUUCACCAAGAGAGACGUCUUCCUGGAGCGGGUGGAACGGCACAUGU | |
| | GUGAUGGGGACAUUGUUAUCUUCAAUaacaggUUGACGACAACAGGCCUGUGACAGCGGGGCUUCGA | |
| | ACUUUCCGUGUUUUUUUUUCUUCCGCUAGUCGAUCAUCCGUGACGGCCGGGCAAGCGCCCGAGUACC | |
| | AGGACCGUCGGGCCCCCGAGAAGGGGGGGCCUAGGAUGCGGCAAGACGACCCGGUUCGGGGAACGCC | |
| | AGCGUGCGCUGGCCGAUCCCGAGGCGAGGUGCCGUAGACCAGUGGACAAUCGACGGAUAACAGCAUA | |
| | UCUAG | |
| 583 | GGGAGACCCUCGACCGUCGAUUGUCCACUGGUCGGCGGAGGGGAGGCUUAAGGUACGUGCUAAACCC | Scytalidium |
| | CCAGCGAUGGGGCCUGUAGGAAAAGCCCUGGUACGGCGAAGCCUACGGGGACCGCCCGAUGGCGGUC | dimidiatum |
| | GGAUGCCGGCGGGCCACGGAGCCCCCGGCGUCGAUUGcUgUUaCCGGCAGCCAACUCUGCACAAAAU | GROUP I |
| | GUCCAUGAUGGGGCAUCGGGUCCGCAUUCUCCCAUGGUCUACCUUUCGCUUGAAUCUUAGUGUGACA | INTRON-PIE-8 |
| | ACUCCGUACAAUGCAGACUUUGACGGGGAUGAGAUGAACUUGCACCUGCCACAGUCUCUGGAGACGC | POLR2A |
| | GAGCAGAGAUCCAGGAGCUGGCCAUGGUUCCUCGCAUGAUUGUCACCCCCCAGAGCAAUCGGCCUGU | precursor |
| | CAUGGGUAUUGUGCAGGACACACUCACAGCAGUGCGCAAAUUCACCAAGAGAGACGUCUUCCUGGAG | |
| | CGGGUGGAACGGCACAUGUGUGAUGGGGACAUUGUUAUCUUCAAUaacaggUUGACGACAACAGGCC | |
| | UGUGACAGCGGGGCUUCGAACUUUCCGUGUUUUUUUUUCUUCCGCUAGUCGAUCAUCCGUGACGGCC | |
| | GGGCAAGCGCCCGAGUACCAGGACCGUCGGGCCCCCGAGAAGGGGGGGCCUAGGAUGCGGCAAGACG | |
| | ACCCGGUUCGGGGAACGCCAGCGUGCGCUGGCCGAUCCCGAGGCGAGGUGCCGUAGCGGGCACCCGU | |
| | CGUAACGCGCGGUAGGGCGUCGGUCCCCCCUCCCACGACCAGUGGACAAUCGACGGAUAACAGCAUA | |
| | UCUAG | |
| 584 | GGGAGACCCUCGACCGUCGAUUGUCCACUGGUCGGAGCCCCCGGCGUCGAUUGcUgUUaCCGGCAGC | Scytalidium |
| | CAACUCUGCACAAAAUGUCCAUGAUGGGGCAUCGGGUCCGCAUUCUCCCAUGGUCUACCUUUCGCUU | dimidiatum |
| | GAAUCUUAGUGUGACAACUCCGUACAAUGCAGACUUUGACGGGGAUGAGAUGAACUUGCACCUGCCA | GROUP I |
| | CAGUCUCUGGAGACGCGAGCAGAGAUCCAGGAGCUGGCCAUGGUUCCUCGCAUGAUUGUCACCCCCC | INTRON-PIE-9 |
| | AGAGCAAUCGGCCUGUCAUGGGUAUUGUGCAGGACACACUCACAGCAGUGCGCAAAUUCACCAAGAG | POLR2A |
| | AGACGUCUUCCUGGAGCGGGUGGAACGGCACAUGUGUGAUGGGGACAUUGUUAUCUUCAAUaacagg | precursor |
| | UUGACGACAACAGGCCUGUGACAGCGGGGCUUCGAACUUUCCGUGUUUUUUUUUCUUCCGCUAGUCG | |
| | AUCAUCCGUGACGGCCGGGCAAGCGCCCGAGUACCAGGACCGUCGGGCCCCCGAGAAGGGGGGGCCU | |
| | AGGAUGCGGCAAGACGACCCGGUUCGGGGAACGCCAGCGUGCGCUGGCCGAUCCCGAGGCGAGGUGC | |
| | CGUAGCGGGCACCCGUCGUAACGCGCGGUAGGGCGUCGGUCCCCCCUCCCACGGCGGAGGGGAGGCU | |
| | UAAGGUACGUGCUAAACCCCCAGCGAUGGGGCCUGUAGGAAAAGCCCUGGUACGGCGAAGCCUACGG | |
| | GGACCGCCCGAUGGCGGUCGGAUGCCGGCGGGCCACGACCAGUGGACAAUCGACGGAUAACAGCAUA | |
| | UCUAG | |
| 585 | gggagacccUcgaCCGUCGAUUGUCCACUGGUCAAUGGAGAAGGUGUAGAGACUGGAAGGCAGGUAC | Scytonema- |
| | CCUAACAGUAAAGCUGAGGGUAAAGGGACAGUCCAGACCACAAACUGGUUAAUCCAGGCAGUGAAAA | hofmani tRNA |
| | CUGUAGAUGGUAAGcaUaacccCCGGCAGCCAACUCUGCACAAAAUGUCCAUGAUGGGGCAUCGGGU | fMet gene |
| | CCGCAUUCUCCCAUGGUCUACCUUUCGCUUGAAUCUUAGUGUGACAACUCCGUACAAUGCAGACUUU | GROUP I |
| | GACGGGGAUGAGAUGAACUUGCACCUGCCACAGUCUCUGGAGACGCGAGCAGAGAUCCAGGAGCUGG | INTRON-PIE-6 |
| | CCAUGGUUCCUCGCAUGAUUGUCACCCCCCAGAGCAAUCGGCCUGUCAUGGGUAUUGUGCAGGACAC | POLR2A |
| | ACUCACAGCAGUGCGCAAAUUCACCAAGAGAGACGUCUUCCUGGAGCGGGUGGAACGGCACAUGUGU | precursor |
| | GAUGGGGACAUUGUUAUCUUCAAgggcUCAACGAGUAAGAUUGACUAAACGCUUAAAAGUUAUUCUU | |
| | ACUAUGGGCGGUACGUAAAGAAACUUACGUAUGUUUACCUGUCAAACUCGGGGAAGCCAAUAGCGCG | |
| | GUAAUCCCGAACCAAGCUCCAGAGACCAGUGGACAAUCGACGGaUaacagcaUaUcUag | |
| 586 | GGGAGACCCUCGACCGUCGAUUGUCCACUGGUCAACUUACGUAUGUUUACCUGUCAAACUCGGGGAA | Scytonema- |
| | GCCAAUAGCGCGGUAAUCCCGAACCAAGCUCCAGAAAUGGAGAAGGUGUAGAGACUGGAAGGCAGGU | hofmani tRNA |
| | ACCCUAACAGUAAAGCUGAGGGUAAAGGGACAGUCCAGACCACAAACUGGUUAAUCCAGGCAGUGAA | fMet gene |
| | AACUGUAGAUGGUAAGCAUAACCCCCGGCAGCCAACUCUGCACAAAAUGUCCAUGAUGGGGCAUCGG | GROUP I |
| | GUCCGCAUUCUCCCAUGGUCUACCUUUCGCUUGAAUCUUAGUGUGACAACUCCGUACAAUGCAGACU | INTRON-PIE-2 |
| | UUGACGGGGAUGAGAUGAACUUGCACCUGCCACAGUCUCUGGAGACGCGAGCAGAGAUCCAGGAGCU | POLR2A |
| | GGCCAUGGUUCCUCGCAUGAUUGUCACCCCCCAGAGCAAUCGGCCUGUCAUGGGUAUUGUGCAGGAC | precursor |
| | ACACUCACAGCAGUGCGCAAAUUCACCAAGAGAGACGUCUUCCUGGAGCGGGUGGAACGGCACAUGU | |
| | GUGAUGGGGACAUUGUUAUCUUCAAGGGCUCAACGAGUAAGAUUGACUAAACGCUUAAAAGUUAUUC | |
| | UUACUAUGGGCGGUACGUAAAGAGACCAGUGGACAAUCGACGGAUAACAGCAUAUCUAG | |
| 587 | GGGAGACCCUCGACCGUCGAUUGUCCACUGGUCAUCCCGAACCAAGCUCCAGAAAUGGAGAAGGUGU | Scytonema- |
| | AGAGACUGGAAGGCAGGUACCCUAACAGUAAAGCUGAGGGUAAAGGGACAGUCCAGACCACAAACUG | hofmani tRNA |
| | GUUAAUCCAGGCAGUGAAAACUGUAGAUGGUAAGCAUAACCCCCGGCAGCCAACUCUGCACAAAAUG | fMet gene |
| | UCCAUGAUGGGGCAUCGGGUCCGCAUUCUCCCAUGGUCUACCUUUCGCUUGAAUCUUAGUGUGACAA | GROUP I |
| | CUCCGUACAAUGCAGACUUUGACGGGGAUGAGAUGAACUUGCACCUGCCACAGUCUCUGGAGACGCG | INTRON-PIE- |
| | AGCAGAGAUCCAGGAGCUGGCCAUGGUUCCUCGCAUGAUUGUCACCCCCCAGAGCAAUCGGCCUGUC | 4-5 POLR2A |
| | AUGGGUAUUGUGCAGGACACACUCACAGCAGUGCGCAAAUUCACCAAGAGAGACGUCUUCCUGGAGC | precursor |
| | GGGUGGAACGGCACAUGUGUGAUGGGGACAUUGUUAUCUUCAAGGGCUCAACGAGUAAGAUUGACUA | |
| | AACGCUUAAAAGUUAUUCUUACUAUGGGCGGUACGUAAAGAAACUUACGUAUGUUUACCUGUCAAAC | |
| | UCGGGGAAGCCAAUAGCGCGGUAGACCAGUGGACAAUCGACGGAUAACAGCAUAUCUAG | |
| 588 | GGGAGACCCUCGACCGUCGAUUGUCCACUGGUCAAUCCAGGCAGUGAAAACUGUAGAUGGUAAGCAU | Scytonema- |
| | AACCCCCGGCAGCCAACUCUGCACAAAAUGUCCAUGAUGGGGCAUCGGGUCCGCAUUCUCCCAUGGU | hofmani tRNA |
| | CUACCUUUCGCUUGAAUCUUAGUGUGACAACUCCGUACAAUGCAGACUUUGACGGGGAUGAGAUGAA | fMet gene |
| | CUUGCACCUGCCACAGUCUCUGGAGACGCGAGCAGAGAUCCAGGAGCUGGCCAUGGUUCCUCGCAUG | GROUP I |
| | AUUGUCACCCCCCAGAGCAAUCGGCCUGUCAUGGGUAUUGUGCAGGACACACUCACAGCAGUGCGCA | INTRON-PIE-9 |
| | AAUUCACCAAGAGAGACGUCUUCCUGGAGCGGGUGGAACGGCACAUGUGUGAUGGGGACAUUGUUAU | POLR2A |
| | CUUCAAGGGCUCAACGAGUAAGAUUGACUAAACGCUUAAAAGUUAUUCUUACUAUGGGCGGUACGUA | precursor |
| | AAGAAACUUACGUAUGUUUACCUGUCAAACUCGGGGAAGCCAAUAGCGCGGUAAUCCCGAACCAAGC | |
| | UCCAGAAAUGGAGAAGGUGUAGAGACUGGAAGGCAGGUACCCUAACAGUAAAGCUGAGGGUAAAGGG | |
| | ACAGUCCAGACCACAAACUGGUUGACCAGUGGACAAUCGACGGAUAACAGCAUAUCUAG | |
| 589 | gggagacccUcgaCCGUCGAUUGUCCACUGGUCAAUCCGGUGAAGGUGUAGAGACUGGAUGGGCAGC | Agrobacterium |
| | ACCUAAAGGCCGCAAGGUCCACGGUGAAGGGACAGUCCAGACCACGAACGCCCUUAUCCGAUUGAGA | tumefaciens |
| | UGGGCGGCGGCGAAAGCCGAAGCGGUAUGaaUccUggggCCGGCAGCCAACUCUGCACAAAAUGUCC | GROUP I |
| | AUGAUGGGGCAUCGGGUCCGCAUUCUCCCAUGGUCUACCUUUCGCUUGAAUCUUAGUGUGACAACUC | INTRON-PIE-6 |
| | CGUACAAUGCAGACUUUGACGGGGAUGAGAUGAACUUGCACCUGCCACAGUCUCUGGAGACGCGAGC | POLR2A |
| | AGAGAUCCAGGAGCUGGCCAUGGUUCCUCGCAUGAUUGUCACCCCCCAGAGCAAUCGGCCUGUCAUG | precursor |
| | GGUAUUGUGCAGGACACACUCACAGCAGUGCGCAAAUUCACCAAGAGAGACGUCUUCCUGGAGCGGG | |
| | UGGAACGGCACAUGUGUGAUGGGGACAUUGUUAUCUUCAAcaggaUUccUAAGGUAAAUUGGGGGUU | |
| | GCGCCCGGAAACGACGCAAUCGAUCUGCUCAAAGUCGGGGAAAGCUUCGCUGGUCUUCCAGUAUGCC | |
| | AAUCCCGAGCCAAGCUCCGGAGAGACCAGUGGACAAUCGACGGaUaacagcaUaUcUag | |
| 590 | AAAAUCCGUUGACCGGCAGCCAACUCUGCACAAAAUGUCCAUGAUGGGGCAUCGGGUCCGCAUUCUC | Anabaena |
| | CCAUGGUCUACCUUUCGCUUGAAUCUUAGUGUGACAACUCCGUACAAUGCAGACUUUGACGGGGAUG | pre-tRNA-Leu |
| | AGAUGAACUUGCACCUGCCACAGUCUCUGGAGACGCGAGCAGAGAUCCAGGAGCUGGCCAUGGUUCC | gene GROUP I |
| | UCGCAUGAUUGUCACCCCCCAGAGCAAUCGGCCUGUCAUGGGUAUUGUGCAGGACACACUCACAGCA | INTRON |
| | GUGCGCAAAUUCACCAAGAGAGACGUCUUCCUGGAGCGGGUGGAACGGCACAUGUGUGAUGGGGACA | POLR2A |
| | UUGUUAUCUUCAAAGACGCUACGGACUU | circRNA |
| 591 | AAUCCGUUGGUGCUGCCGGCAGCCAACUCUGCACAAAAUGUCCAUGAUGGGGCAUCGGGUCCGCAUU | Azoarcus |
| | CUCCCAUGGUCUACCUUUCGCUUGAAUCUUAGUGUGACAACUCCGUACAAUGCAGACUUUGACGGGG | olearius |
| | AUGAGAUGAACUUGCACCUGCCACAGUCUCUGGAGACGCGAGCAGAGAUCCAGGAGCUGGCCAUGGU | BH72 GROUP I |
| | UCCUCGCAUGAUUGUCACCCCCCAGAGCAAUCGGCCUGUCAUGGGUAUUGUGCAGGACACACUCACA | INTRON-PIE-2 |
| | GCAGUGCGCAAAUUCACCAAGAGAGACGUCUUCCUGGAGCGGGUGGAACGGCACAUGUGUGAUGGGG | POLR2A |
| | ACAUUGUUAUCUUCAAGAGCAGCGGACUCAU | circRNA |
| 592 | AACUUUUAGUUAUCCGGCAGCCAACUCUGCACAAAAUGUCCAUGAUGGGGCAUCGGGUCCGCAUUCU | Staphylococcus |
| | CCCAUGGUCUACCUUUCGCUUGAAUCUUAGUGUGACAACUCCGUACAAUGCAGACUUUGACGGGGAU | phage |
| | GAGAUGAACUUGCACCUGCCACAGUCUCUGGAGACGCGAGCAGAGAUCCAGGAGCUGGCCAUGGUUC | Twort GROUP |
| | CUCGCAUGAUUGUCACCCCCCAGAGCAAUCGGCCUGUCAUGGGUAUUGUGCAGGACACACUCACAGC | I INTRON- |
| | AGUGCGCAAAUUCACCAAGAGAGACGUCUUCCUGGAGCGGGUGGAACGGCACAUGUGUGAUGGGGAC | PIE-2 POLR2A |
| | AUUGUUAUCUUCAAACUACUGAAAGCAU | circRNA |
| 593 | CUGUUACCGGCAGCCAACUCUGCACAAAAUGUCCAUGAUGGGGCAUCGGGUCCGCAUUCUCCCAUGG | Scytalidium |
| | UCUACCUUUCGCUUGAAUCUUAGUGUGACAACUCCGUACAAUGCAGACUUUGACGGGGAUGAGAUGA | dimidiatum |
| | ACUUGCACCUGCCACAGUCUCUGGAGACGCGAGCAGAGAUCCAGGAGCUGGCCAUGGUUCCUCGCAU | GROUP I |
| | GAUUGUCACCCCCCAGAGCAAUCGGCCUGUCAUGGGUAUUGUGCAGGACACACUCACAGCAGUGCGC | INTRON-PIE-2 |
| | AAAUUCACCAAGAGAGACGUCUUCCUGGAGCGGGUGGAACGGCACAUGUGUGAUGGGGACAUUGUUA | POLR2A |
| | UCUUCAAUAACAGGU | circRNA |
| 594 | CAUAACCCCCGGCAGCCAACUCUGCACAAAAUGUCCAUGAUGGGGCAUCGGGUCCGCAUUCUCCCAU | Scytonema- |
| | GGUCUACCUUUCGCUUGAAUCUUAGUGUGACAACUCCGUACAAUGCAGACUUUGACGGGGAUGAGAU | hofmani tRNA |
| | GAACUUGCACCUGCCACAGUCUCUGGAGACGCGAGCAGAGAUCCAGGAGCUGGCCAUGGUUCCUCGC | fMet gene |
| | AUGAUUGUCACCCCCCAGAGCAAUCGGCCUGUCAUGGGUAUUGUGCAGGACACACUCACAGCAGUGC | GROUP I |
| | GCAAAUUCACCAAGAGAGACGUCUUCCUGGAGCGGGUGGAACGGCACAUGUGUGAUGGGGACAUUGU | INTRON-PIE-6 |
| | UAUCUUCAAGGGCU | POLR2A |
| | | circRNA |
| 595 | AAUCCUGGGGCCGGCAGCCAACUCUGCACAAAAUGUCCAUGAUGGGGCAUCGGGUCCGCAUUCUCCC | Agrobacterium |
| | AUGGUCUACCUUUCGCUUGAAUCUUAGUGUGACAACUCCGUACAAUGCAGACUUUGACGGGGAUGAG | tumefaciens |
| | AUGAACUUGCACCUGCCACAGUCUCUGGAGACGCGAGCAGAGAUCCAGGAGCUGGCCAUGGUUCCUC | GROUP I |
| | GCAUGAUUGUCACCCCCCAGAGCAAUCGGCCUGUCAUGGGUAUUGUGCAGGACACACUCACAGCAGU | INTRON-PIE-6 |
| | GCGCAAAUUCACCAAGAGAGACGUCUUCCUGGAGCGGGUGGAACGGCACAUGUGUGAUGGGGACAUU | POLR2A |
| | GUUAUCUUCAACAGGAUUCCU | circRNA |
| 596 | GGGAGACCCUCGACCGUCGAUUGUCCACUGGUCAACAAUAGAUGACUUACAACUAAUCGGAAGGUGC | AnaX-CAV2- |
| | AGAGACUCGACGGGAGCUACCCUAACGUCAAGACGAGGGUAAAGAGAGAGUCCAAUUCUCAAAGCCA | Luciferase |
| | AUAGGCAGUAGCGAAAGCUGCAAGAGAAUGAAAAUCCGUUGAUUAAAACAGCCUGUGGGUUGCACCC | precursor |
| | ACCCACAGGGCCCACUGGGCGCUAGCACUCUGGUAUCACGAUACCUUUGUGCGCCUGUUUUAUAUCC | |
| | CCACCCCGAGUAAACGUUAGAAGUUACGCAACCCCGAUCAAUAGUAGGUGUAGCACUCCAGCUGCAU | |
| | CGAGAUCAAGCACUUCUGUCUCCCCGGACCGAGUAUCAAUAGACUGCUAACGCGGUUGAAGGAGAAA | |
| | ACGUUCGUUACCCGGCCAAUUACUUCGAGAAGCCCAGUAGUGCCGUGAAAGUUGCGGAGUGUUUCGC | |
| | UCAGCACUUCCCCCGUGUAGAUCAGGCUGAUGAGUCACCGCGAUCCCCACAGGUGACUGUGGCGGUG | |
| | GCUGCGUUGGCGGCCUGCCUAUGGGGCAACCCAUAGGACGCUCUAAUACAGACAUGGUGCGAAGAGC | |
| | CUAUUGAGCUAAUUGGUAGUCCUCCGGCCCCUGAAUGCGGCUAAUCCUAACUGCGGAGCACAUGCCC | |
| | UCAAACCAGGGGGUGGUGUGUCGUAACGGGUAACUCUGCAGCGGAACCGACUACUUUGGGUGUCCGU | |
| | GUUUCUUUUUAUUCUUAUAAUGGCUGCUUAUGGUGACAAUUAAAGAAUUGUUACCAUAUAGCUAUUG | |
| | GAUUGGCCAUCCGGUGACUAACAAAUCGCUCAUAUACCAGUUUGUUGGUUUUGUUCCCUUAUCACAU | |
| | ACAGCUCAUAACACCCUCUUAUAUUUACUACAAUUGAAUAGCAAGAAAUGGAGGACGCCAAGAACAU | |
| | CAAGAAGGGCCCGGCGCCCUUCUACCCGCUGGAGGACGGGACCGCCGGCGAGCAGCUCCACAAGGCC | |
| | AUGAAGCGGUACGCCCUGGUGCCGGGCACGAUCGCCUUCACCGACGCCCACAUCGAGGUCGACAUCA | |
| | CCUACGCGGAGUACUUCGAGAUGAGCGUGCGCCUGGCCGAGGCCAUGAAGCGGUACGGCCUGAACAC | |
| | CAACCACCGGAUCGUGGUGUGCUCGGAGAACAGCCUGCAGUUCUUCAUGCCGGUGCUGGGCGCCCUC | |
| | UUCAUCGGCGUGGCCGUCGCCCCGGCGAACGACAUCUACAACGAGCGGGAGCUGCUGAACAGCAUGG | |
| | GGAUCAGCCAGCCGACCGUGGUGUUCGUGAGCAAGAAGGGCCUGCAGAAGAUCCUGAACGUGCAGAA | |
| | GAAGCUGCCCAUCAUCCAGAAGAUCAUCAUCAUGGACAGCAAGACCGACUACCAGGGCUUCCAGUCG | |
| | AUGUACACGUUCGUGACCAGCCACCUCCCGCCGGGCUUCAACGAGUACGACUUCGUCCCGGAGAGCU | |
| | UCGACCGGGACAAGACCAUCGCCCUGAUCAUGAACAGCAGCGGCAGCACCGGCCUGCCGAAGGGGGU | |
| | GGCCCUGCCGCACCGGACCGCCUGCGUGCGCUUCUCGCACGCCCGGGACCCCAUCUUCGGCAACCAG | |
| | AUCAUCCCGGACACCGCCAUCCUGAGCGUGGUGCCGUUCCACCACGGCUUCGGCAUGUUCACGACCC | |
| | UGGGCUACCUCAUCUGCGGCUUCCGGGUGGUCCUGAUGUACCGGUUCGAGGAGGAGCUGUUCCUGCG | |
| | GAGCCUGCAGGACUACAAGAUCCAGAGCGCGCUGCUCGUGCCGACCCUGUUCAGCUUCUUCGCCAAG | |
| | AGCACCCUGAUCGACAAGUACGACCUGUCGAACCUGCACGAGAUCGCCAGCGGGGGCGCCCCGCUGA | |
| | GCAAGGAGGUGGGCGAGGCCGUGGCCAAGCGGUUCCACCUCCCGGGCAUCCGCCAGGGCUACGGCCU | |
| | GACCGAGACCACGAGCGCGAUCCUGAUCACCCCCGAGGGGGACGACAAGCCGGGCGCCGUGGGCAAG | |
| | GUGGUCCCGUUCUUCGAGGCCAAGGUGGUGGACCUGGACACCGGCAAGACCCUGGGCGUGAACCAGC | |
| | GGGGCGAGCUGUGCGUGCGGGGGCCGAUGAUCAUGAGCGGCUACGUGAACAACCCGGAGGCCACCAA | |
| | CGCCCUCAUCGACAAGGACGGCUGGCUGCACAGCGGCGACAUCGCCUACUGGGACGAGGACGAGCAC | |
| | UUCUUCAUCGUCGACCGGCUGAAGUCGCUGAUCAAGUACAAGGGCUACCAGGUGGCGCCGGCCGAGC | |
| | UGGAGAGCAUCCUGCUCCAGCACCCCAACAUCUUCGACGCCGGCGUGGCCGGGCUGCCGGACGACGA | |
| | CGCCGGCGAGCUGCCGGCCGCGGUGGUGGUGCUGGAGCACGGCAAGACCAUGACGGAGAAGGAGAUC | |
| | GUCGACUACGUGGCCAGCCAGGUGACCACCGCCAAGAAGCUGCGGGGCGGCGUGGUGUUCGUGGACG | |
| | AGGUCCCGAAGGGCCUGACCGGGAAGCUCGACGCCCGGAAGAUCCGCGAGAUCCUGAUCAAGGCCAA | |
| | GAAGGGCGGCAAGAUCGCCGUGAGCUAAAGACGCUACGGACUUAAAUAAUUGAGCCUUAAAGAAGAA | |
| | AUUCUUUAAGUGGAUGCUCUCAAACUCAGGGAAACCUAAAUCUAGUUAUAGACAAGGCAAUCCUGAG | |
| | CCAAGCCGAAGUAGUAAUUAGUAAGACCAGUGGACAAUCGACGGAUAACAGCAUAUCUAG | |
| 597 | GGGAGACCCUCGACCGUCGAUUGUCCACUGGUCAAUGGAGAAGGUGUAGAGACUGGAAGGCAGGUAC | Scytonema- |
| | CCUAACAGUAAAGCUGAGGGUAAAGGGACAGUCCAGACCACAAACUGGUUAAUCCAGGCAGUGAAAA | hofmani tRNA |
| | CUGUAGAUGGUAAGCAUAACCCUUAAAACAGCCUGUGGGUUGCACCCACCCACAGGGCCCACUGGGC | fMet gene |
| | GCUAGCACUCUGGUAUCACGAUACCUUUGUGCGCCUGUUUUAUAUCCCCACCCCGAGUAAACGUUAG | GROUP I |
| | AAGUUACGCAACCCCGAUCAAUAGUAGGUGUAGCACUCCAGCUGCAUCGAGAUCAAGCACUUCUGUC | INTRON-PIE-6 |
| | UCCCCGGACCGAGUAUCAAUAGACUGCUAACGCGGUUGAAGGAGAAAACGUUCGUUACCCGGCCAAU | CAV2- |
| | UACUUCGAGAAGCCCAGUAGUGCCGUGAAAGUUGCGGAGUGUUUCGCUCAGCACUUCCCCCGUGUAG | Luciferase |
| | AUCAGGCUGAUGAGUCACCGCGAUCCCCACAGGUGACUGUGGCGGUGGCUGCGUUGGCGGCCUGCCU | precursor |
| | AUGGGGCAACCCAUAGGACGCUCUAAUACAGACAUGGUGCGAAGAGCCUAUUGAGCUAAUUGGUAGU | |
| | CCUCCGGCCCCUGAAUGCGGCUAAUCCUAACUGCGGAGCACAUGCCCUCAAACCAGGGGGUGGUGUG | |
| | UCGUAACGGGUAACUCUGCAGCGGAACCGACUACUUUGGGUGUCCGUGUUUCUUUUUAUUCUUAUAA | |
| | UGGCUGCUUAUGGUGACAAUUAAAGAAUUGUUACCAUAUAGCUAUUGGAUUGGCCAUCCGGUGACUA | |
| | ACAAAUCGCUCAUAUACCAGUUUGUUGGUUUUGUUCCCUUAUCACAUACAGCUCAUAACACCCUCUU | |
| | AUAUUUACUACAAUUGAAUAGCAAGAAGCCACCAUGGAGGACGCCAAGAACAUCAAGAAGGGCCCGG | |
| | CGCCCUUCUACCCGCUGGAGGACGGGACCGCCGGCGAGCAGCUCCACAAGGCCAUGAAGCGGUACGC | |
| | CCUGGUGCCGGGCACGAUCGCCUUCACCGACGCCCACAUCGAGGUCGACAUCACCUACGCGGAGUAC | |
| | UUCGAGAUGAGCGUGCGCCUGGCCGAGGCCAUGAAGCGGUACGGCCUGAACACCAACCACCGGAUCG | |
| | UGGUGUGCUCGGAGAACAGCCUGCAGUUCUUCAUGCCGGUGCUGGGCGCCCUCUUCAUCGGCGUGGC | |
| | CGUCGCCCCGGCGAACGACAUCUACAACGAGCGGGAGCUGCUGAACAGCAUGGGGAUCAGCCAGCCG | |
| | ACCGUGGUGUUCGUGAGCAAGAAGGGCCUGCAGAAGAUCCUGAACGUGCAGAAGAAGCUGCCCAUCA | |
| | UCCAGAAGAUCAUCAUCAUGGACAGCAAGACCGACUACCAGGGCUUCCAGUCGAUGUACACGUUCGU | |
| | GACCAGCCACCUCCCGCCGGGCUUCAACGAGUACGACUUCGUCCCGGAGAGCUUCGACCGGGACAAG | |
| | ACCAUCGCCCUGAUCAUGAACAGCAGCGGCAGCACCGGCCUGCCGAAGGGGGUGGCCCUGCCGCACC | |
| | GGACCGCCUGCGUGCGCUUCUCGCACGCCCGGGACCCCAUCUUCGGCAACCAGAUCAUCCCGGACAC | |
| | CGCCAUCCUGAGCGUGGUGCCGUUCCACCACGGCUUCGGCAUGUUCACGACCCUGGGCUACCUCAUC | |
| | UGCGGCUUCCGGGUGGUCCUGAUGUACCGGUUCGAGGAGGAGCUGUUCCUGCGGAGCCUGCAGGACU | |
| | ACAAGAUCCAGAGCGCGCUGCUCGUGCCGACCCUGUUCAGCUUCUUCGCCAAGAGCACCCUGAUCGA | |
| | CAAGUACGACCUGUCGAACCUGCACGAGAUCGCCAGCGGGGGCGCCCCGCUGAGCAAGGAGGUGGGC | |
| | GAGGCCGUGGCCAAGCGGUUCCACCUCCCGGGCAUCCGCCAGGGCUACGGCCUGACCGAGACCACGA | |
| | GCGCGAUCCUGAUCACCCCCGAGGGGGACGACAAGCCGGGCGCCGUGGGCAAGGUGGUCCCGUUCUU | |
| | CGAGGCCAAGGUGGUGGACCUGGACACCGGCAAGACCCUGGGCGUGAACCAGCGGGGCGAGCUGUGC | |
| | GUGCGGGGGCCGAUGAUCAUGAGCGGCUACGUGAACAACCCGGAGGCCACCAACGCCCUCAUCGACA | |
| | AGGACGGCUGGCUGCACAGCGGCGACAUCGCCUACUGGGACGAGGACGAGCACUUCUUCAUCGUCGA | |
| | CCGGCUGAAGUCGCUGAUCAAGUACAAGGGCUACCAGGUGGCGCCGGCCGAGCUGGAGAGCAUCCUG | |
| | CUCCAGCACCCCAACAUCUUCGACGCCGGCGUGGCCGGGCUGCCGGACGACGACGCCGGCGAGCUGC | |
| | CGGCCGCGGUGGUGGUGCUGGAGCACGGCAAGACCAUGACGGAGAAGGAGAUCGUCGACUACGUGGC | |
| | CAGCCAGGUGACCACCGCCAAGAAGCUGCGGGGCGGCGUGGUGUUCGUGGACGAGGUCCCGAAGGGC | |
| | CUGACCGGGAAGCUCGACGCCCGGAAGAUCCGCGAGAUCCUGAUCAAGGCCAAGAAGGGCGGCAAGA | |
| | UCGCCGUGAGCUGAUAAGGGCUCAACGAGUAAGAUUGACUAAACGCUUAAAAGUUAUUCUUACUAUG | |
| | GGCGGUACGUAAAGAAACUUACGUAUGUUUACCUGUCAAACUCGGGGAAGCCAAUAGCGCGGUAAUC | |
| 598 | CCGAACCAAGCUCCAGAGACCAGUGGACAAUCGACGGAUAACAGCAUAUCUAG | |
| | AAAAUCCGUUGAUUAAAACAGCCUGUGGGUUGCACCCACCCACAGGGCCCACUGGGCGCUAGCACUC | AnaX-CAV2- |
| | UGGUAUCACGAUACCUUUGUGCGCCUGUUUUAUAUCCCCACCCCGAGUAAACGUUAGAAGUUACGCA | Luciferase |
| | ACCCCGAUCAAUAGUAGGUGUAGCACUCCAGCUGCAUCGAGAUCAAGCACUUCUGUCUCCCCGGACC | circRNA |
| | GAGUAUCAAUAGACUGCUAACGCGGUUGAAGGAGAAAACGUUCGUUACCCGGCCAAUUACUUCGAGA | |
| | AGCCCAGUAGUGCCGUGAAAGUUGCGGAGUGUUUCGCUCAGCACUUCCCCCGUGUAGAUCAGGCUGA | |
| | UGAGUCACCGCGAUCCCCACAGGUGACUGUGGCGGUGGCUGCGUUGGCGGCCUGCCUAUGGGGCAAC | |
| | CCAUAGGACGCUCUAAUACAGACAUGGUGCGAAGAGCCUAUUGAGCUAAUUGGUAGUCCUCCGGCCC | |
| | CUGAAUGCGGCUAAUCCUAACUGCGGAGCACAUGCCCUCAAACCAGGGGGUGGUGUGUCGUAACGGG | |
| | UAACUCUGCAGCGGAACCGACUACUUUGGGUGUCCGUGUUUCUUUUUAUUCUUAUAAUGGCUGCUUA | |
| | UGGUGACAAUUAAAGAAUUGUUACCAUAUAGCUAUUGGAUUGGCCAUCCGGUGACUAACAAAUCGCU | |
| | CAUAUACCAGUUUGUUGGUUUUGUUCCCUUAUCACAUACAGCUCAUAACACCCUCUUAUAUUUACUA | |
| | CAAUUGAAUAGCAAGAAAUGGAGGACGCCAAGAACAUCAAGAAGGGCCCGGCGCCCUUCUACCCGCU | |
| | GGAGGACGGGACCGCCGGCGAGCAGCUCCACAAGGCCAUGAAGCGGUACGCCCUGGUGCCGGGCACG | |
| | AUCGCCUUCACCGACGCCCACAUCGAGGUCGACAUCACCUACGCGGAGUACUUCGAGAUGAGCGUGC | |
| | GCCUGGCCGAGGCCAUGAAGCGGUACGGCCUGAACACCAACCACCGGAUCGUGGUGUGCUCGGAGAA | |
| | CAGCCUGCAGUUCUUCAUGCCGGUGCUGGGCGCCCUCUUCAUCGGCGUGGCCGUCGCCCCGGCGAAC | |
| | GACAUCUACAACGAGCGGGAGCUGCUGAACAGCAUGGGGAUCAGCCAGCCGACCGUGGUGUUCGUGA | |
| | GCAAGAAGGGCCUGCAGAAGAUCCUGAACGUGCAGAAGAAGCUGCCCAUCAUCCAGAAGAUCAUCAU | |
| | CAUGGACAGCAAGACCGACUACCAGGGCUUCCAGUCGAUGUACACGUUCGUGACCAGCCACCUCCCG | |
| | CCGGGCUUCAACGAGUACGACUUCGUCCCGGAGAGCUUCGACCGGGACAAGACCAUCGCCCUGAUCA | |
| | UGAACAGCAGCGGCAGCACCGGCCUGCCGAAGGGGGUGGCCCUGCCGCACCGGACCGCCUGCGUGCG | |
| | CUUCUCGCACGCCCGGGACCCCAUCUUCGGCAACCAGAUCAUCCCGGACACCGCCAUCCUGAGCGUG | |
| | GUGCCGUUCCACCACGGCUUCGGCAUGUUCACGACCCUGGGCUACCUCAUCUGCGGCUUCCGGGUGG | |
| | UCCUGAUGUACCGGUUCGAGGAGGAGCUGUUCCUGCGGAGCCUGCAGGACUACAAGAUCCAGAGCGC | |
| | GCUGCUCGUGCCGACCCUGUUCAGCUUCUUCGCCAAGAGCACCCUGAUCGACAAGUACGACCUGUCG | |
| | AACCUGCACGAGAUCGCCAGCGGGGGCGCCCCGCUGAGCAAGGAGGUGGGCGAGGCCGUGGCCAAGC | |
| | GGUUCCACCUCCCGGGCAUCCGCCAGGGCUACGGCCUGACCGAGACCACGAGCGCGAUCCUGAUCAC | |
| | CCCCGAGGGGGACGACAAGCCGGGCGCCGUGGGCAAGGUGGUCCCGUUCUUCGAGGCCAAGGUGGUG | |
| | GACCUGGACACCGGCAAGACCCUGGGCGUGAACCAGCGGGGCGAGCUGUGCGUGCGGGGGCCGAUGA | |
| | UCAUGAGCGGCUACGUGAACAACCCGGAGGCCACCAACGCCCUCAUCGACAAGGACGGCUGGCUGCA | |
| | CAGCGGCGACAUCGCCUACUGGGACGAGGACGAGCACUUCUUCAUCGUCGACCGGCUGAAGUCGCUG | |
| | AUCAAGUACAAGGGCUACCAGGUGGCGCCGGCCGAGCUGGAGAGCAUCCUGCUCCAGCACCCCAACA | |
| | UCUUCGACGCCGGCGUGGCCGGGCUGCCGGACGACGACGCCGGCGAGCUGCCGGCCGCGGUGGUGGU | |
| | GCUGGAGCACGGCAAGACCAUGACGGAGAAGGAGAUCGUCGACUACGUGGCCAGCCAGGUGACCACC | |
| | GCCAAGAAGCUGCGGGGCGGCGUGGUGUUCGUGGACGAGGUCCCGAAGGGCCUGACCGGGAAGCUCG | |
| | ACGCCCGGAAGAUCCGCGAGAUCCUGAUCAAGGCCAAGAAGGGCGGCAAGAUCGCCGUGAGCUAAAG | |
| | ACGCUACGGACUU | |
| 599 | CAUAACCCUUAAAACAGCCUGUGGGUUGCACCCACCCACAGGGCCCACUGGGCGCUAGCACUCUGGU | Scytonema- |
| | AUCACGAUACCUUUGUGCGCCUGUUUUAUAUCCCCACCCCGAGUAAACGUUAGAAGUUACGCAACCC | hofmani tRNA |
| | CGAUCAAUAGUAGGUGUAGCACUCCAGCUGCAUCGAGAUCAAGCACUUCUGUCUCCCCGGACCGAGU | fMet gene |
| | AUCAAUAGACUGCUAACGCGGUUGAAGGAGAAAACGUUCGUUACCCGGCCAAUUACUUCGAGAAGCC | GROUP I |
| | CAGUAGUGCCGUGAAAGUUGCGGAGUGUUUCGCUCAGCACUUCCCCCGUGUAGAUCAGGCUGAUGAG | INTRON-PIE-6 |
| | UCACCGCGAUCCCCACAGGUGACUGUGGCGGUGGCUGCGUUGGCGGCCUGCCUAUGGGGCAACCCAU | CAV2- |
| | AGGACGCUCUAAUACAGACAUGGUGCGAAGAGCCUAUUGAGCUAAUUGGUAGUCCUCCGGCCCCUGA | Luciferase |
| | AUGCGGCUAAUCCUAACUGCGGAGCACAUGCCCUCAAACCAGGGGGUGGUGUGUCGUAACGGGUAAC | circRNA |
| | UCUGCAGCGGAACCGACUACUUUGGGUGUCCGUGUUUCUUUUUAUUCUUAUAAUGGCUGCUUAUGGU | |
| | GACAAUUAAAGAAUUGUUACCAUAUAGCUAUUGGAUUGGCCAUCCGGUGACUAACAAAUCGCUCAUA | |
| | UACCAGUUUGUUGGUUUUGUUCCCUUAUCACAUACAGCUCAUAACACCCUCUUAUAUUUACUACAAU | |
| | UGAAUAGCAAGAAGCCACCAUGGAGGACGCCAAGAACAUCAAGAAGGGCCCGGCGCCCUUCUACCCG | |
| | CUGGAGGACGGGACCGCCGGCGAGCAGCUCCACAAGGCCAUGAAGCGGUACGCCCUGGUGCCGGGCA | |
| | CGAUCGCCUUCACCGACGCCCACAUCGAGGUCGACAUCACCUACGCGGAGUACUUCGAGAUGAGCGU | |
| | GCGCCUGGCCGAGGCCAUGAAGCGGUACGGCCUGAACACCAACCACCGGAUCGUGGUGUGCUCGGAG | |
| | AACAGCCUGCAGUUCUUCAUGCCGGUGCUGGGCGCCCUCUUCAUCGGCGUGGCCGUCGCCCCGGCGA | |
| | ACGACAUCUACAACGAGCGGGAGCUGCUGAACAGCAUGGGGAUCAGCCAGCCGACCGUGGUGUUCGU | |
| | GAGCAAGAAGGGCCUGCAGAAGAUCCUGAACGUGCAGAAGAAGCUGCCCAUCAUCCAGAAGAUCAUC | |
| | AUCAUGGACAGCAAGACCGACUACCAGGGCUUCCAGUCGAUGUACACGUUCGUGACCAGCCACCUCC | |
| | CGCCGGGCUUCAACGAGUACGACUUCGUCCCGGAGAGCUUCGACCGGGACAAGACCAUCGCCCUGAU | |
| | CAUGAACAGCAGCGGCAGCACCGGCCUGCCGAAGGGGGUGGCCCUGCCGCACCGGACCGCCUGCGUG | |
| | CGCUUCUCGCACGCCCGGGACCCCAUCUUCGGCAACCAGAUCAUCCCGGACACCGCCAUCCUGAGCG | |
| | UGGUGCCGUUCCACCACGGCUUCGGCAUGUUCACGACCCUGGGCUACCUCAUCUGCGGCUUCCGGGU | |
| | GGUCCUGAUGUACCGGUUCGAGGAGGAGCUGUUCCUGCGGAGCCUGCAGGACUACAAGAUCCAGAGC | |
| | GCGCUGCUCGUGCCGACCCUGUUCAGCUUCUUCGCCAAGAGCACCCUGAUCGACAAGUACGACCUGU | |
| | CGAACCUGCACGAGAUCGCCAGCGGGGGCGCCCCGCUGAGCAAGGAGGUGGGCGAGGCCGUGGCCAA | |
| | GCGGUUCCACCUCCCGGGCAUCCGCCAGGGCUACGGCCUGACCGAGACCACGAGCGCGAUCCUGAUC | |
| | ACCCCCGAGGGGGACGACAAGCCGGGCGCCGUGGGCAAGGUGGUCCCGUUCUUCGAGGCCAAGGUGG | |
| | UGGACCUGGACACCGGCAAGACCCUGGGCGUGAACCAGCGGGGCGAGCUGUGCGUGCGGGGGCCGAU | |
| | GAUCAUGAGCGGCUACGUGAACAACCCGGAGGCCACCAACGCCCUCAUCGACAAGGACGGCUGGCUG | |
| | CACAGCGGCGACAUCGCCUACUGGGACGAGGACGAGCACUUCUUCAUCGUCGACCGGCUGAAGUCGC | |
| | UGAUCAAGUACAAGGGCUACCAGGUGGCGCCGGCCGAGCUGGAGAGCAUCCUGCUCCAGCACCCCAA | |
| | CAUCUUCGACGCCGGCGUGGCCGGGCUGCCGGACGACGACGCCGGCGAGCUGCCGGCCGCGGUGGUG | |
| | GUGCUGGAGCACGGCAAGACCAUGACGGAGAAGGAGAUCGUCGACUACGUGGCCAGCCAGGUGACCA | |
| | CCGCCAAGAAGCUGCGGGGCGGCGUGGUGUUCGUGGACGAGGUCCCGAAGGGCCUGACCGGGAAGCU | |
| | CGACGCCCGGAAGAUCCGCGAGAUCCUGAUCAAGGCCAAGAAGGGCGGCAAGAUCGCCGUGAGCUGA | |
| | UAAGGGCU | |
| 600 | CCGAUUAGUUGUAAGUCAUCUAUUG | Ligand |
| | | Intron |
| 601 | UCAACGGAUUUUAAGUCCGUAGCGUCU | Ligand |
| | | Feature |
| 602 | AUUUUAAGUC | Ligand F10 |
| 603 | AACGGAUUUUAAGUCCGUAG | Ligand F20 |
| 604 | CAACGGAUUUUAAGUCCGUAGCG | Ligand F23 |
| 605 | UCAACGGAUUUUAAGUCCGUAGCGU | Ligand F25 |
| 606 | UCAACGGAUUUUAAGUCCGUAGCGUCU | Ligand F27 |
| 607 | AUCAACGGAUUUUAAGUCCGUAGCGUCUU | Ligand F29 |
| 608 | UGCGAGGAACCAUCCGGCCAGCUCCUG | Ligand- |
| | | Feature/ |
| | | ligase |
| 609 | GAUAGUCGUUAGGCAUUUAUGUAGA | Ligand-Td- |
| | | Intron |
| 610 | UAUUAAACGGUAGACCCAAGAAAACAU | Ligand-Td- |
| | | Feature |
| 611 | GGGAGACCCUCGAGGCCCCUCUGCAGUGUCUGUUGUGGAGGCCAGAUACGUAACUGCUUCCGCAACA | circRNA-1X |
| | AUAGAUGACUUACAACUAAUCGGAAGGUGCAGAGACUCGACGGGAGCUACCCUAACGUCAAGACGAG | miRNA-122-5P |
| | GGUAAAGAGAGAGUCCAAUUCUCAAAGCCAAUAGGCAGUAGCGAAAGCUGCAAGAGAAUGAAAACCC | |
| | CCCCACCCACAGCAAGAAUGCCAUCAUCUGUCCUCACCCCCAAUUUUCCCUUUUCUUCCCCUGCAAC | |
| | CAUUACGCUUACUCGCAUGUGCAUUGAGUGGUGCAUGUGUUGAACAAACAGCUACACUCACAUGGGG | |
| | GCGGGUUUUCCCGCCCUACGGCCUCUCGCGAGGCCCACCCCUUCCCUCCCCUUAUAACUACAGUGCU | |
| | UUGGUAGGUAAGCAUCCUGAUCCCCCGCGGAAGCUGCUCACGUGGCAACUGUGGGGACCCAGACAGG | |
| | UUAUCAAAGGCACCCGGUCUUUCCGCCUUCAGGAGUAUCCCUACUAGUGAAUUCUAGCGGGGCUCUG | |
| | CUUGGUGCCAACCUCCCCCAAAUGCGCGCUGCGGGAGUGCUCUUCCCCAACUCACCCUAGUAUCCUC | |
| | UCAUGUGUGUGCUUGGUCAGCAUAUCUGAGACGAUGUUCCGCUGUCCCAGACCAGUCCAGUAAUGGA | |
| | CGGGCCAGUGCGUGUAGUCGUCUUCCGGCUUGUCCGGGGCAUGUUUGGUGAACCGGUGGGGUAAGGU | |
| | UGGUGUGCCCAACGCCCGUACUUUGGUGACACCUCAAGACCACCCAGGAAUGCCAGGGAGGUACCCC | |
| | ACCUCACGGUGGGAUCUGACCCUGGGCUAAUUGUCUACGGUGGUUCUUCUUGCUUCCACUUCUUUCU | |
| | UCUGUUCACGGCCACCAUGGAGGACGCCAAGAACAUCAAGAAGGGCCCGGCGCCCUUCUACCCGCUG | |
| | GAGGACGGGACCGCCGGCGAGCAGCUCCACAAGGCCAUGAAGCGGUACGCCCUGGUGCCGGGCACGA | |
| | UCGCCUUCACCGACGCCCACAUCGAGGUCGACAUCACCUACGCGGAGUACUUCGAGAUGAGCGUGCG | |
| | CCUGGCCGAGGCCAUGAAGCGGUACGGCCUGAACACCAACCACCGGAUCGUGGUGUGCUCGGAGAAC | |
| | AGCCUGCAGUUCUUCAUGCCGGUGCUGGGCGCCCUCUUCAUCGGCGUGGCCGUCGCCCCGGCGAACG | |
| | ACAUCUACAACGAGCGGGAGCUGCUGAACAGCAUGGGGAUCAGCCAGCCGACCGUGGUGUUCGUGAG | |
| | CAAGAAGGGCCUGCAGAAGAUCCUGAACGUGCAGAAGAAGCUGCCCAUCAUCCAGAAGAUCAUCAUC | |
| | AUGGACAGCAAGACCGACUACCAGGGCUUCCAGUCGAUGUACACGUUCGUGACCAGCCACCUCCCGC | |
| | CGGGCUUCAACGAGUACGACUUCGUCCCGGAGAGCUUCGACCGGGACAAGACCAUCGCCCUGAUCAU | |
| | GAACAGCAGCGGCAGCACCGGCCUGCCGAAGGGGGUGGCCCUGCCGCACCGGACCGCCUGCGUGCGC | |
| | UUCUCGCACGCCCGGGACCCCAUCUUCGGCAACCAGAUCAUCCCGGACACCGCCAUCCUGAGCGUGG | |
| | UGCCGUUCCACCACGGCUUCGGCAUGUUCACGACCCUGGGCUACCUCAUCUGCGGCUUCCGGGUGGU | |
| | CCUGAUGUACCGGUUCGAGGAGGAGCUGUUCCUGCGGAGCCUGCAGGACUACAAGAUCCAGAGCGCG | |
| | CUGCUCGUGCCGACCCUGUUCAGCUUCUUCGCCAAGAGCACCCUGAUCGACAAGUACGACCUGUCGA | |
| | ACCUGCACGAGAUCGCCAGCGGGGGCGCCCCGCUGAGCAAGGAGGUGGGCGAGGCCGUGGCCAAGCG | |
| | GUUCCACCUCCCGGGCAUCCGCCAGGGCUACGGCCUGACCGAGACCACGAGCGCGAUCCUGAUCACC | |
| | CCCGAGGGGGACGACAAGCCGGGCGCCGUGGGCAAGGUGGUCCCGUUCUUCGAGGCCAAGGUGGUGG | |
| | ACCUGGACACCGGCAAGACCCUGGGCGUGAACCAGCGGGGCGAGCUGUGCGUGCGGGGGCCGAUGAU | |
| | CAUGAGCGGCUACGUGAACAACCCGGAGGCCACCAACGCCCUCAUCGACAAGGACGGCUGGCUGCAC | |
| | AGCGGCGACAUCGCCUACUGGGACGAGGACGAGCACUUCUUCAUCGUCGACCGGCUGAAGUCGCUGA | |
| | UCAAGUACAAGGGCUACCAGGUGGCGCCGGCCGAGCUGGAGAGCAUCCUGCUCCAGCACCCCAACAU | |
| | CUUCGACGCCGGCGUGGCCGGGCUGCCGGACGACGACGCCGGCGAGCUGCCGGCCGCGGUGGUGGUG | |
| | CUGGAGCACGGCAAGACCAUGACGGAGAAGGAGAUCGUCGACUACGUGGCCAGCCAGGUGACCACCG | |
| | CCAAGAAGCUGCGGGGCGGCGUGGUGUUCGUGGACGAGGUCCCGAAGGGCCUGACCGGGAAGCUCGA | |
| | CGCCCGGAAGAUCCGCGAGAUCCUGAUCAAGGCCAAGAAGGGCGGCAAGAUCGCCGUGAGCUGAUAA | |
| | CGUUAAAUCACAAACACCAUUGUCACACUCCAACUUAAAUCCAGACGGGGGGGGCUUAAAUAAUUGA | |
| | GCCUUAAAGAAGAAAUUCUUUAAGUGGAUGCUCUCAAACUCAGGGAAACCUAAAUCUAGUUAUAGAC | |
| | AAGGCAAUCCUGAGCCAAGCCGAAGUAGUAAUUAGUAAGCGGAAGCAGUUACGUAUCUGGCCUCCAC | |
| | AACAGACACUGCAGAGGGGCCAUAACAGCAUAUCUAG | |
| 612 | GGGAGACCCUCGAGGCCCCUCUGCAGUGUCUGUUGUGGAGGCCAGAUACGUAACUGCUUCCGCAACA | circRNA-2X |
| | AUAGAUGACUUACAACUAAUCGGAAGGUGCAGAGACUCGACGGGAGCUACCCUAACGUCAAGACGAG | miRNA-122-5P |
| | GGUAAAGAGAGAGUCCAAUUCUCAAAGCCAAUAGGCAGUAGCGAAAGCUGCAAGAGAAUGAAAACCC | |
| | CCCCACCCACAGCAAGAAUGCCAUCAUCUGUCCUCACCCCCAAUUUUCCCUUUUCUUCCCCUGCAAC | |
| | CAUUACGCUUACUCGCAUGUGCAUUGAGUGGUGCAUGUGUUGAACAAACAGCUACACUCACAUGGGG | |
| | GCGGGUUUUCCCGCCCUACGGCCUCUCGCGAGGCCCACCCCUUCCCUCCCCUUAUAACUACAGUGCU | |
| | UUGGUAGGUAAGCAUCCUGAUCCCCCGCGGAAGCUGCUCACGUGGCAACUGUGGGGACCCAGACAGG | |
| | UUAUCAAAGGCACCCGGUCUUUCCGCCUUCAGGAGUAUCCCUACUAGUGAAUUCUAGCGGGGCUCUG | |
| | CUUGGUGCCAACCUCCCCCAAAUGCGCGCUGCGGGAGUGCUCUUCCCCAACUCACCCUAGUAUCCUC | |
| | UCAUGUGUGUGCUUGGUCAGCAUAUCUGAGACGAUGUUCCGCUGUCCCAGACCAGUCCAGUAAUGGA | |
| | CGGGCCAGUGCGUGUAGUCGUCUUCCGGCUUGUCCGGGGCAUGUUUGGUGAACCGGUGGGGUAAGGU | |
| | UGGUGUGCCCAACGCCCGUACUUUGGUGACACCUCAAGACCACCCAGGAAUGCCAGGGAGGUACCCC | |
| | ACCUCACGGUGGGAUCUGACCCUGGGCUAAUUGUCUACGGUGGUUCUUCUUGCUUCCACUUCUUUCU | |
| | UCUGUUCACGGCCACCAUGGAGGACGCCAAGAACAUCAAGAAGGGCCCGGCGCCCUUCUACCCGCUG | |
| | GAGGACGGGACCGCCGGCGAGCAGCUCCACAAGGCCAUGAAGCGGUACGCCCUGGUGCCGGGCACGA | |
| | UCGCCUUCACCGACGCCCACAUCGAGGUCGACAUCACCUACGCGGAGUACUUCGAGAUGAGCGUGCG | |
| | CCUGGCCGAGGCCAUGAAGCGGUACGGCCUGAACACCAACCACCGGAUCGUGGUGUGCUCGGAGAAC | |
| | AGCCUGCAGUUCUUCAUGCCGGUGCUGGGCGCCCUCUUCAUCGGCGUGGCCGUCGCCCCGGCGAACG | |
| | ACAUCUACAACGAGCGGGAGCUGCUGAACAGCAUGGGGAUCAGCCAGCCGACCGUGGUGUUCGUGAG | |
| | CAAGAAGGGCCUGCAGAAGAUCCUGAACGUGCAGAAGAAGCUGCCCAUCAUCCAGAAGAUCAUCAUC | |
| | AUGGACAGCAAGACCGACUACCAGGGCUUCCAGUCGAUGUACACGUUCGUGACCAGCCACCUCCCGC | |
| | CGGGCUUCAACGAGUACGACUUCGUCCCGGAGAGCUUCGACCGGGACAAGACCAUCGCCCUGAUCAU | |
| | GAACAGCAGCGGCAGCACCGGCCUGCCGAAGGGGGUGGCCCUGCCGCACCGGACCGCCUGCGUGCGC | |
| | UUCUCGCACGCCCGGGACCCCAUCUUCGGCAACCAGAUCAUCCCGGACACCGCCAUCCUGAGCGUGG | |
| | UGCCGUUCCACCACGGCUUCGGCAUGUUCACGACCCUGGGCUACCUCAUCUGCGGCUUCCGGGUGGU | |
| | CCUGAUGUACCGGUUCGAGGAGGAGCUGUUCCUGCGGAGCCUGCAGGACUACAAGAUCCAGAGCGCG | |
| | CUGCUCGUGCCGACCCUGUUCAGCUUCUUCGCCAAGAGCACCCUGAUCGACAAGUACGACCUGUCGA | |
| | ACCUGCACGAGAUCGCCAGCGGGGGCGCCCCGCUGAGCAAGGAGGUGGGCGAGGCCGUGGCCAAGCG | |
| | GUUCCACCUCCCGGGCAUCCGCCAGGGCUACGGCCUGACCGAGACCACGAGCGCGAUCCUGAUCACC | |
| | CCCGAGGGGGACGACAAGCCGGGCGCCGUGGGCAAGGUGGUCCCGUUCUUCGAGGCCAAGGUGGUGG | |
| | ACCUGGACACCGGCAAGACCCUGGGCGUGAACCAGCGGGGCGAGCUGUGCGUGCGGGGGCCGAUGAU | |
| | CAUGAGCGGCUACGUGAACAACCCGGAGGCCACCAACGCCCUCAUCGACAAGGACGGCUGGCUGCAC | |
| | AGCGGCGACAUCGCCUACUGGGACGAGGACGAGCACUUCUUCAUCGUCGACCGGCUGAAGUCGCUGA | |
| | UCAAGUACAAGGGCUACCAGGUGGCGCCGGCCGAGCUGGAGAGCAUCCUGCUCCAGCACCCCAACAU | |
| | CUUCGACGCCGGCGUGGCCGGGCUGCCGGACGACGACGCCGGCGAGCUGCCGGCCGCGGUGGUGGUG | |
| | CUGGAGCACGGCAAGACCAUGACGGAGAAGGAGAUCGUCGACUACGUGGCCAGCCAGGUGACCACCG | |
| | CCAAGAAGCUGCGGGGCGGCGUGGUGUUCGUGGACGAGGUCCCGAAGGGCCUGACCGGGAAGCUCGA | |
| | CGCCCGGAAGAUCCGCGAGAUCCUGAUCAAGGCCAAGAAGGGCGGCAAGAUCGCCGUGAGCUGAUAA | |
| | GAUUAUUUCGCAAACACCAUUGUCACACUCCAACUUAAAUCCCAAACACCAUUGUCACACUCCAACU | |
| | UAAAUCCAGACGGGGGGGGCUUAAAUAAUUGAGCCUUAAAGAAGAAAUUCUUUAAGUGGAUGCUCUC | |
| | AAACUCAGGGAAACCUAAAUCUAGUUAUAGACAAGGCAAUCCUGAGCCAAGCCGAAGUAGUAAUUAG | |
| | UAAGCGGAAGCAGUUACGUAUCUGGCCUCCACAACAGACACUGCAGAGGGGCCAUAACAGCAUAUCU | |
| | AG | |
| 613 | GGGAGACCCUCGAGGCCCCUCUGCAGUGUCUGUUGUGGAGGCCAGAUACGUAACUGCUUCCGCAACA | circRNA-1X |
| | AUAGAUGACUUACAACUAAUCGGAAGGUGCAGAGACUCGACGGGAGCUACCCUAACGUCAAGACGAG | miRNA-671-5P |
| | GGUAAAGAGAGAGUCCAAUUCUCAAAGCCAAUAGGCAGUAGCGAAAGCUGCAAGAGAAUGAAAACCC | |
| | CCCCACCCACAGCAAGAAUGCCAUCAUCUGUCCUCACCCCCAAUUUUCCCUUUUCUUCCCCUGCAAC | |
| | CAUUACGCUUACUCGCAUGUGCAUUGAGUGGUGCAUGUGUUGAACAAACAGCUACACUCACAUGGGG | |
| | GCGGGUUUUCCCGCCCUACGGCCUCUCGCGAGGCCCACCCCUUCCCUCCCCUUAUAACUACAGUGCU | |
| | UUGGUAGGUAAGCAUCCUGAUCCCCCGCGGAAGCUGCUCACGUGGCAACUGUGGGGACCCAGACAGG | |
| | UUAUCAAAGGCACCCGGUCUUUCCGCCUUCAGGAGUAUCCCUACUAGUGAAUUCUAGCGGGGCUCUG | |
| | CUUGGUGCCAACCUCCCCCAAAUGCGCGCUGCGGGAGUGCUCUUCCCCAACUCACCCUAGUAUCCUC | |
| | UCAUGUGUGUGCUUGGUCAGCAUAUCUGAGACGAUGUUCCGCUGUCCCAGACCAGUCCAGUAAUGGA | |
| | CGGGCCAGUGCGUGUAGUCGUCUUCCGGCUUGUCCGGGGCAUGUUUGGUGAACCGGUGGGGUAAGGU | |
| | UGGUGUGCCCAACGCCCGUACUUUGGUGACACCUCAAGACCACCCAGGAAUGCCAGGGAGGUACCCC | |
| | ACCUCACGGUGGGAUCUGACCCUGGGCUAAUUGUCUACGGUGGUUCUUCUUGCUUCCACUUCUUUCU | |
| | UCUGUUCACGGCCACCAUGGAGGACGCCAAGAACAUCAAGAAGGGCCCGGCGCCCUUCUACCCGCUG | |
| | GAGGACGGGACCGCCGGCGAGCAGCUCCACAAGGCCAUGAAGCGGUACGCCCUGGUGCCGGGCACGA | |
| | UCGCCUUCACCGACGCCCACAUCGAGGUCGACAUCACCUACGCGGAGUACUUCGAGAUGAGCGUGCG | |
| | CCUGGCCGAGGCCAUGAAGCGGUACGGCCUGAACACCAACCACCGGAUCGUGGUGUGCUCGGAGAAC | |
| | AGCCUGCAGUUCUUCAUGCCGGUGCUGGGCGCCCUCUUCAUCGGCGUGGCCGUCGCCCCGGCGAACG | |
| | ACAUCUACAACGAGCGGGAGCUGCUGAACAGCAUGGGGAUCAGCCAGCCGACCGUGGUGUUCGUGAG | |
| | CAAGAAGGGCCUGCAGAAGAUCCUGAACGUGCAGAAGAAGCUGCCCAUCAUCCAGAAGAUCAUCAUC | |
| | AUGGACAGCAAGACCGACUACCAGGGCUUCCAGUCGAUGUACACGUUCGUGACCAGCCACCUCCCGC | |
| | CGGGCUUCAACGAGUACGACUUCGUCCCGGAGAGCUUCGACCGGGACAAGACCAUCGCCCUGAUCAU | |
| | GAACAGCAGCGGCAGCACCGGCCUGCCGAAGGGGGUGGCCCUGCCGCACCGGACCGCCUGCGUGCGC | |
| | UUCUCGCACGCCCGGGACCCCAUCUUCGGCAACCAGAUCAUCCCGGACACCGCCAUCCUGAGCGUGG | |
| | UGCCGUUCCACCACGGCUUCGGCAUGUUCACGACCCUGGGCUACCUCAUCUGCGGCUUCCGGGUGGU | |
| | CCUGAUGUACCGGUUCGAGGAGGAGCUGUUCCUGCGGAGCCUGCAGGACUACAAGAUCCAGAGCGCG | |
| | CUGCUCGUGCCGACCCUGUUCAGCUUCUUCGCCAAGAGCACCCUGAUCGACAAGUACGACCUGUCGA | |
| | ACCUGCACGAGAUCGCCAGCGGGGGCGCCCCGCUGAGCAAGGAGGUGGGCGAGGCCGUGGCCAAGCG | |
| | GUUCCACCUCCCGGGCAUCCGCCAGGGCUACGGCCUGACCGAGACCACGAGCGCGAUCCUGAUCACC | |
| | CCCGAGGGGGACGACAAGCCGGGCGCCGUGGGCAAGGUGGUCCCGUUCUUCGAGGCCAAGGUGGUGG | |
| | ACCUGGACACCGGCAAGACCCUGGGCGUGAACCAGCGGGGCGAGCUGUGCGUGCGGGGGCCGAUGAU | |
| | CAUGAGCGGCUACGUGAACAACCCGGAGGCCACCAACGCCCUCAUCGACAAGGACGGCUGGCUGCAC | |
| | AGCGGCGACAUCGCCUACUGGGACGAGGACGAGCACUUCUUCAUCGUCGACCGGCUGAAGUCGCUGA | |
| | UCAAGUACAAGGGCUACCAGGUGGCGCCGGCCGAGCUGGAGAGCAUCCUGCUCCAGCACCCCAACAU | |
| | CUUCGACGCCGGCGUGGCCGGGCUGCCGGACGACGACGCCGGCGAGCUGCCGGCCGCGGUGGUGGUG | |
| | CUGGAGCACGGCAAGACCAUGACGGAGAAGGAGAUCGUCGACUACGUGGCCAGCCAGGUGACCACCG | |
| | CCAAGAAGCUGCGGGGCGGCGUGGUGUUCGUGGACGAGGUCCCGAAGGGCCUGACCGGGAAGCUCGA | |
| | CGCCCGGAAGAUCCGCGAGAUCCUGAUCAAGGCCAAGAAGGGCGGCAAGAUCGCCGUGAGCUGAUAA | |
| | CGUUAAAUCACUCCAGCCCCUCCAGGGCUUCCUACUUAAAUCCAGACGGGGGGGGCUUAAAUAAUUG | |
| | AGCCUUAAAGAAGAAAUUCUUUAAGUGGAUGCUCUCAAACUCAGGGAAACCUAAAUCUAGUUAUAGA | |
| | CAAGGCAAUCCUGAGCCAAGCCGAAGUAGUAAUUAGUAAGCGGAAGCAGUUACGUAUCUGGCCUCCA | |
| | CAACAGACACUGCAGAGGGGCCAUAACAGCAUAUCUAG | |
| 614 | GGGAGACCCUCGAGGCCCCUCUGCAGUGUCUGUUGUGGAGGCCAGAUACGUAACUGCUUCCGCAACA | circRNA-2X |
| | AUAGAUGACUUACAACUAAUCGGAAGGUGCAGAGACUCGACGGGAGCUACCCUAACGUCAAGACGAG | miRNA-671-5P |
| | GGUAAAGAGAGAGUCCAAUUCUCAAAGCCAAUAGGCAGUAGCGAAAGCUGCAAGAGAAUGAAAACCC | |
| | CCCCACCCACAGCAAGAAUGCCAUCAUCUGUCCUCACCCCCAAUUUUCCCUUUUCUUCCCCUGCAAC | |
| | CAUUACGCUUACUCGCAUGUGCAUUGAGUGGUGCAUGUGUUGAACAAACAGCUACACUCACAUGGGG | |
| | GCGGGUUUUCCCGCCCUACGGCCUCUCGCGAGGCCCACCCCUUCCCUCCCCUUAUAACUACAGUGCU | |
| | UUGGUAGGUAAGCAUCCUGAUCCCCCGCGGAAGCUGCUCACGUGGCAACUGUGGGGACCCAGACAGG | |
| | UUAUCAAAGGCACCCGGUCUUUCCGCCUUCAGGAGUAUCCCUACUAGUGAAUUCUAGCGGGGCUCUG | |
| | CUUGGUGCCAACCUCCCCCAAAUGCGCGCUGCGGGAGUGCUCUUCCCCAACUCACCCUAGUAUCCUC | |
| | UCAUGUGUGUGCUUGGUCAGCAUAUCUGAGACGAUGUUCCGCUGUCCCAGACCAGUCCAGUAAUGGA | |
| | CGGGCCAGUGCGUGUAGUCGUCUUCCGGCUUGUCCGGGGCAUGUUUGGUGAACCGGUGGGGUAAGGU | |
| | UGGUGUGCCCAACGCCCGUACUUUGGUGACACCUCAAGACCACCCAGGAAUGCCAGGGAGGUACCCC | |
| | ACCUCACGGUGGGAUCUGACCCUGGGCUAAUUGUCUACGGUGGUUCUUCUUGCUUCCACUUCUUUCU | |
| | UCUGUUCACGGCCACCAUGGAGGACGCCAAGAACAUCAAGAAGGGCCCGGCGCCCUUCUACCCGCUG | |
| | GAGGACGGGACCGCCGGCGAGCAGCUCCACAAGGCCAUGAAGCGGUACGCCCUGGUGCCGGGCACGA | |
| | UCGCCUUCACCGACGCCCACAUCGAGGUCGACAUCACCUACGCGGAGUACUUCGAGAUGAGCGUGCG | |
| | CCUGGCCGAGGCCAUGAAGCGGUACGGCCUGAACACCAACCACCGGAUCGUGGUGUGCUCGGAGAAC | |
| | AGCCUGCAGUUCUUCAUGCCGGUGCUGGGCGCCCUCUUCAUCGGCGUGGCCGUCGCCCCGGCGAACG | |
| | ACAUCUACAACGAGCGGGAGCUGCUGAACAGCAUGGGGAUCAGCCAGCCGACCGUGGUGUUCGUGAG | |
| | CAAGAAGGGCCUGCAGAAGAUCCUGAACGUGCAGAAGAAGCUGCCCAUCAUCCAGAAGAUCAUCAUC | |
| | AUGGACAGCAAGACCGACUACCAGGGCUUCCAGUCGAUGUACACGUUCGUGACCAGCCACCUCCCGC | |
| | CGGGCUUCAACGAGUACGACUUCGUCCCGGAGAGCUUCGACCGGGACAAGACCAUCGCCCUGAUCAU | |
| | GAACAGCAGCGGCAGCACCGGCCUGCCGAAGGGGGUGGCCCUGCCGCACCGGACCGCCUGCGUGCGC | |
| | UUCUCGCACGCCCGGGACCCCAUCUUCGGCAACCAGAUCAUCCCGGACACCGCCAUCCUGAGCGUGG | |
| | UGCCGUUCCACCACGGCUUCGGCAUGUUCACGACCCUGGGCUACCUCAUCUGCGGCUUCCGGGUGGU | |
| | CCUGAUGUACCGGUUCGAGGAGGAGCUGUUCCUGCGGAGCCUGCAGGACUACAAGAUCCAGAGCGCG | |
| | CUGCUCGUGCCGACCCUGUUCAGCUUCUUCGCCAAGAGCACCCUGAUCGACAAGUACGACCUGUCGA | |
| | ACCUGCACGAGAUCGCCAGCGGGGGCGCCCCGCUGAGCAAGGAGGUGGGCGAGGCCGUGGCCAAGCG | |
| | GUUCCACCUCCCGGGCAUCCGCCAGGGCUACGGCCUGACCGAGACCACGAGCGCGAUCCUGAUCACC | |
| | CCCGAGGGGGACGACAAGCCGGGCGCCGUGGGCAAGGUGGUCCCGUUCUUCGAGGCCAAGGUGGUGG | |
| | ACCUGGACACCGGCAAGACCCUGGGCGUGAACCAGCGGGGCGAGCUGUGCGUGCGGGGGCCGAUGAU | |
| | CAUGAGCGGCUACGUGAACAACCCGGAGGCCACCAACGCCCUCAUCGACAAGGACGGCUGGCUGCAC | |
| | AGCGGCGACAUCGCCUACUGGGACGAGGACGAGCACUUCUUCAUCGUCGACCGGCUGAAGUCGCUGA | |
| | UCAAGUACAAGGGCUACCAGGUGGCGCCGGCCGAGCUGGAGAGCAUCCUGCUCCAGCACCCCAACAU | |
| | CUUCGACGCCGGCGUGGCCGGGCUGCCGGACGACGACGCCGGCGAGCUGCCGGCCGCGGUGGUGGUG | |
| | CUGGAGCACGGCAAGACCAUGACGGAGAAGGAGAUCGUCGACUACGUGGCCAGCCAGGUGACCACCG | |
| | CCAAGAAGCUGCGGGGCGGCGUGGUGUUCGUGGACGAGGUCCCGAAGGGCCUGACCGGGAAGCUCGA | |
| | CGCCCGGAAGAUCCGCGAGAUCCUGAUCAAGGCCAAGAAGGGCGGCAAGAUCGCCGUGAGCUGAUAA | |
| | GAUUAUUUCGCUCCAGCCCCUCCAGGGCUUCCUACUUAAAUCCCUCCAGCCCCUCCAGGGCUUCCUA | |
| | CUUAAAUCCAGACGGGGGGGGCUUAAAUAAUUGAGCCUUAAAGAAGAAAUUCUUUAAGUGGAUGCUC | |
| | UCAAACUCAGGGAAACCUAAAUCUAGUUAUAGACAAGGCAAUCCUGAGCCAAGCCGAAGUAGUAAUU | |
| | AGUAAGCGGAAGCAGUUACGUAUCUGGCCUCCACAACAGACACUGCAGAGGGGCCAUAACAGCAUAU | |
| | CUAG | |
| 615 | GGGAGACCCUCGACCGUCGAUUGUCCACUGGUCCCGUCGAUUGUCCACUGGUCAACAAUAGAUGACU | circRBD-WT |
| | UACAACUAAUCGGAAGGUGCAGAGACUCGACGGGAGCUACCCUAACGUCAAGACGAGGGUAAAGAGA | |
| | GAGUCCAAUUCUCAAAGCCAAUAGGCAGUAGCGAAAGCUGCAAGAGAAUGAAAACCCCCCCACCCAC | |
| | AGCAAGAAUGCCAUCAUCUGUCCUCACCCCCAAUUUUCCCUUUUCUUCCCCUGCAACCAUUACGCUU | |
| | ACUCGCAUGUGCAUUGAGUGGUGCAUGUGUUGAACAAACAGCUACACUCACAUGGGGGCGGGUUUUC | |
| | CCGCCCUACGGCCUCUCGCGAGGCCCACCCCUUCCCUCCCCUUAUAACUACAGUGCUUUGGUAGGUA | |
| | AGCAUCCUGAUCCCCCGCGGAAGCUGCUCACGUGGCAACUGUGGGGACCCAGACAGGUUAUCAAAGG | |
| | CACCCGGUCUUUCCGCCUUCAGGAGUAUCCCUACUAGUGAAUUCUAGCGGGGCUCUGCUUGGUGCCA | |
| | ACCUCCCCCAAAUGCGCGCUGCGGGAGUGCUCUUCCCCAACUCACCCUAGUAUCCUCUCAUGUGUGU | |
| | GCUUGGUCAGCAUAUCUGAGACGAUGUUCCGCUGUCCCAGACCAGUCCAGUAAUGGACGGGCCAGUG | |
| | CGUGUAGUCGUCUUCCGGCUUGUCCGGGGCAUGUUUGGUGAACCGGUGGGGUAAGGUUGGUGUGCCC | |
| | AACGCCCGUACUUUGGUGACACCUCAAGACCACCCAGGAAUGCCAGGGAGGUACCCCACCUCACGGU | |
| | GGGAUCUGACCCUGGGCUAAUUGUCUACGGUGGUUCUUCUUGCUUCCACUUCUUUCUUCUGUUCACG | |
| | GCCACCAUGGAUAUGAGAGCCCAUGUUCAUCUGCUGGGCCUGCUGCUGCUUUGGCUGCCUGGCGCUA | |
| | AAUGCAACAUCACCAACCUGUGUCCAUUUGGAGAGGUGUUCAAUGCCACCAGGUUUGCCUCUGUCUA | |
| | UGCCUGGAACAGGAAGAGGAUUAGCAACUGUGUGGCUGACUACUCUGUGCUCUACAACUCUGCCUCC | |
| | UUCAGCACCUUCAAGUGUUAUGGAGUGAGCCCAACCAAACUGAAUGACCUGUGUUUCACCAAUGUCU | |
| | AUGCUGACUCCUUUGUGAUUAGGGGAGAUGAGGUGAGACAGAUUGCCCCUGGACAAACAGGCAAGAU | |
| | UGCUGACUACAACUACAAACUGCCUGAUGACUUCACAGGCUGUGUGAUUGCCUGGAACAGCAACAAC | |
| | CUGGACAGCAAGGUGGGAGGCAACUACAACUACCUCUACAGACUGUUCAGGAAGAGCAACCUGAAAC | |
| | CAUUUGAGAGGGACAUCAGCACAGAGAUUUACCAGGCUGGCAGCACACCAUGUAAUGGAGUGGAGGG | |
| | CUUCAACUGUUACUUUCCACUCCAAUCCUAUGGCUUCCAACCAACCAAUGGAGUGGGCUACCAACCA | |
| | UACAGGGUGGUGGUGCUGUCCUUUGAACUGCUCCAUGCCCCUGCCACAGUGGAGCCUAAGAGCAGCG | |
| | ACAAGACCCACACAUGCCCUCCAUGUCCUGCUCCAGAACUGCUCGGAGGCCCUUCCGUGUUCCUGUU | |
| | UCCUCCAAAGCCUAAGGACACCCUGAUGAUCUCCAGAACACCCGAAGUGACCUGCGUGGUGGUCGAU | |
| | GUGUCUCACGAGGACCCUGAAGUGAAGUUCAAUUGGUACGUCGACGGCGUGGAAGUGCACAACGCCA | |
| | AGACCAAGCCUAGAGAGGAACAGUACAACAGCACCUACAGAGUGGUGUCCGUGCUGACCGUGCUGCA | |
| | CCAGGAUUGGCUGAACGGCAAAGAGUACAAGUGCAAGGUGUCCAACAAGGCCCUGCCAGCUCCUAUC | |
| | GAGAAAACCAUCAGCAAGGCCAAGGGCCAGCCUAGGGAACCCCAGGUUUACACACUGCCUCCAAGCC | |
| | GGGAAGAGAUGACCAAGAACCAGGUGUCCCUGACCUGCCUGGUCAAGGGCUUCUACCCUUCCGAUAU | |
| | CGCCGUGGAAUGGGAGAGCAAUGGCCAGCCUGAGAACAACUACAAGACCACACCUCCUGUGCUGGAC | |
| | AGCGACGGCUCAUUCUUCCUGUACAGCAAGCUGACAGUGGACAAGAGCAGAUGGCAGCAGGGCAACG | |
| | UGUUCAGCUGCAGCGUGAUGCACGAGGCCCUGCACAACCACUACACCCAGAAGUCCCUGAGCCUGUC | |
| | UCCUGGCAAAUGAUAAAGACGGGGGGGGCUUAAAUAAUUGAGCCUUAAAGAAGAAAUUCUUUAAGUG | |
| | GAUGCUCUCAAACUCAGGGAAACCUAAAUCUAGUUAUAGACAAGGCAAUCCUGAGCCAAGCCGAAGU | |
| | AGUAAUUAGUAAGACCAGUGGACAAUCGACGGGACCAGUGGACAAUCGACGGAUAACAGCAUAUCUA | |
| | G | |
| 616 | GGGAGACCCUCGACCGUCGAUUGUCCACUGGUCCCGUCGAUUGUCCACUGGUCAACAAUAGAUGACU | circRBD-UTR- |
| | UACAACUAAUCGGAAGGUGCAGAGACUCGACGGGAGCUACCCUAACGUCAAGACGAGGGUAAAGAGA | 1 |
| | GAGUCCAAUUCUCAAAGCCAAUAGGCAGUAGCGAAAGCUGCAAGAGAAUGAAAACCCCCCCAAAAAA | |
| | AAAAAAACCAAAAAAAAAAAACAAAAAAAAAAAAUAAUUGACUAACCCACAGCAAGAAUGCCAUCAU | |
| | CUGUCCUCACCCCCAAUUUUCCCUUUUCUUCCCCUGCAACCAUUACGCUUACUCGCAUGUGCAUUGA | |
| | GUGGUGCAUGUGUUGAACAAACAGCUACACUCACAUGGGGGGCGGUUUUCCCGCCCUACGGCCUCUC | |
| | GCGAGGCCCACCCCUUCCCUCCCCUUAUAACUACAGUGCUUUGGUAGGUAAGCAUCCUGAUCCCCCG | |
| | CGGAAGCUGCUCACGUGGCAACUGUGGGGACCCAGACAGGUUAUCAAAGGCACCCGGUCUUUCCGCC | |
| | UUCAGGAGUAUCCCUACUAGUGAAUUCUAGCGGGGCUCUGCUUGGUGCCAACCUCCCCCAAAUGCGC | |
| | GCUGCGGGAGUGCUCUUCCCCAACUCACCCUAGUAUCCUCUCAUGUGUGUGCUUGGUCAGCAUAUCU | |
| | GAGACGAUGUUCCGCUGUCCCAGACCAGUCCAGUAAUGGACGGGCCAGUGCGUGUAGUCGUCUUCCG | |
| | GCUUGUCCGGGGCAUGUUUGGUGAACCGGUGGGGUAAGGUUGGUGUGCCCAACGCCCGUACUUUGGU | |
| | GACACCUCAAGACCACCCAGGAAUGCCAGGGAGGUACCCCACCUCACGGUGGGAUCUGACCCUGGGC | |
| | UAAUUGUCUACGGUGGUUCUUCUUGCUUCCACUUCUUUCUUCUGUUCACGGCCACCAUGGAUAUGAG | |
| | AGCCCAUGUUCAUCUGCUGGGCCUGCUGCUGCUUUGGCUGCCUGGCGCUAAAUGCAACAUCACCAAC | |
| | CUGUGUCCAUUUGGAGAGGUGUUCAAUGCCACCAGGUUUGCCUCUGUCUAUGCCUGGAACAGGAAGA | |
| | GGAUUAGCAACUGUGUGGCUGACUACUCUGUGCUCUACAACUCUGCCUCCUUCAGCACCUUCAAGUG | |
| | UUAUGGAGUGAGCCCAACCAAACUGAAUGACCUGUGUUUCACCAAUGUCUAUGCUGACUCCUUUGUG | |
| | AUUAGGGGAGAUGAGGUGAGACAGAUUGCCCCUGGACAAACAGGCAAGAUUGCUGACUACAACUACA | |
| | AACUGCCUGAUGACUUCACAGGCUGUGUGAUUGCCUGGAACAGCAACAACCUGGACAGCAAGGUGGG | |
| | AGGCAACUACAACUACCUCUACAGACUGUUCAGGAAGAGCAACCUGAAACCAUUUGAGAGGGACAUC | |
| | AGCACAGAGAUUUACCAGGCUGGCAGCACACCAUGUAAUGGAGUGGAGGGCUUCAACUGUUACUUUC | |
| | CACUCCAAUCCUAUGGCUUCCAACCAACCAAUGGAGUGGGCUACCAACCAUACAGGGUGGUGGUGCU | |
| | GUCCUUUGAACUGCUCCAUGCCCCUGCCACAGUGGAGCCUAAGAGCAGCGACAAGACCCACACAUGC | |
| | CCUCCAUGUCCUGCUCCAGAACUGCUCGGAGGCCCUUCCGUGUUCCUGUUUCCUCCAAAGCCUAAGG | |
| | ACACCCUGAUGAUCUCCAGAACACCCGAAGUGACCUGCGUGGUGGUCGAUGUGUCUCACGAGGACCC | |
| | UGAAGUGAAGUUCAAUUGGUACGUCGACGGCGUGGAAGUGCACAACGCCAAGACCAAGCCUAGAGAG | |
| | GAACAGUACAACAGCACCUACAGAGUGGUGUCCGUGCUGACCGUGCUGCACCAGGAUUGGCUGAACG | |
| | GCAAAGAGUACAAGUGCAAGGUGUCCAACAAGGCCCUGCCAGCUCCUAUCGAGAAAACCAUCAGCAA | |
| | GGCCAAGGGCCAGCCUAGGGAACCCCAGGUUUACACACUGCCUCCAAGCCGGGAAGAGAUGACCAAG | |
| | AACCAGGUGUCCCUGACCUGCCUGGUCAAGGGCUUCUACCCUUCCGAUAUCGCCGUGGAAUGGGAGA | |
| | GCAAUGGCCAGCCUGAGAACAACUACAAGACCACACCUCCUGUGCUGGACAGCGACGGCUCAUUCUU | |
| | CCUGUACAGCAAGCUGACAGUGGACAAGAGCAGAUGGCAGCAGGGCAACGUGUUCAGCUGCAGCGUG | |
| | AUGCACGAGGCCCUGCACAACCACUACACCCAGAAGUCCCUGAGCCUGUCUCCUGGCAAAUGAUAAA | |
| | GACGGGGGGGGCUUAAAUAAUUGAGCCUUAAAGAAGAAAUUCUUUAAGUGGAUGCUCUCAAACUCAG | |
| | GGAAACCUAAAUCUAGUUAUAGACAAGGCAAUCCUGAGCCAAGCCGAAGUAGUAAUUAGUAAGACCA | |
| | GUGGACAAUCGACGGGACCAGUGGACAAUCGACGGAUAACAGCAUAUCUAG | |
| 617 | GGGAGACCCUCGACCGUCGAUUGUCCACUGGUCCCGUCGAUUGUCCACUGGUCAACAAUAGAUGACU | circRBD-UTR- |
| | UACAACUAAUCGGAAGGUGCAGAGACUCGACGGGAGCUACCCUAACGUCAAGACGAGGGUAAAGAGA | 2 |
| | GAGUCCAAUUCUCAAAGCCAAUAGGCAGUAGCGAAAGCUGCAAGAGAAUGAAAACCCCCCCAGGGAA | |
| | AUAAGAGAGAAAAGAAGAGUAAGAAGAAAUAUAAGACCCCGGCGCCCCCACAGCAAGAAUGCCAUCA | |
| | UCUGUCCUCACCCCCAAUUUUCCCUUUUCUUCCCCUGCAACCAUUACGCUUACUCGCAUGUGCAUUG | |
| | AGUGGUGCAUGUGUUGAACAAACAGCUACACUCACAUGGGGGCGGGUUUUCCCGCCCUACGGCCUCU | |
| | CGCGAGGCCCACCCCUUCCCUCCCCUUAUAACUACAGUGCUUUGGUAGGUAAGCAUCCUGAUCCCCC | |
| | GCGGAAGCUGCUCACGUGGCAACUGUGGGGACCCAGACAGGUUAUCAAAGGCACCCGGUCUUUCCGC | |
| | CUUCAGGAGUAUCCCUACUAGUGAAUUCUAGCGGGGCUCUGCUUGGUGCCAACCUCCCCCAAAUGCG | |
| | CGCUGCGGGAGUGCUCUUCCCCAACUCACCCUAGUAUCCUCUCAUGUGUGUGCUUGGUCAGCAUAUC | |
| | UGAGACGAUGUUCCGCUGUCCCAGACCAGUCCAGUAAUGGACGGGCCAGUGCGUGUAGUCGUCUUCC | |
| | GGCUUGUCCGGGGCAUGUUUGGUGAACCGGUGGGGUAAGGUUGGUGUGCCCAACGCCCGUACUUUGG | |
| | UGACACCUCAAGACCACCCAGGAAUGCCAGGGAGGUACCCCACCUCACGGUGGGAUCUGACCCUGGG | |
| | CUAAUUGUCUACGGUGGUUCUUCUUGCUUCCACUUCUUUCUUCUGUUCACGGCCACCAUGGAUAUGA | |
| | GAGCCCAUGUUCAUCUGCUGGGCCUGCUGCUGCUUUGGCUGCCUGGCGCUAAAUGCAACAUCACCAA | |
| | CCUGUGUCCAUUUGGAGAGGUGUUCAAUGCCACCAGGUUUGCCUCUGUCUAUGCCUGGAACAGGAAG | |
| | AGGAUUAGCAACUGUGUGGCUGACUACUCUGUGCUCUACAACUCUGCCUCCUUCAGCACCUUCAAGU | |
| | GUUAUGGAGUGAGCCCAACCAAACUGAAUGACCUGUGUUUCACCAAUGUCUAUGCUGACUCCUUUGU | |
| | GAUUAGGGGAGAUGAGGUGAGACAGAUUGCCCCUGGACAAACAGGCAAGAUUGCUGACUACAACUAC | |
| | AAACUGCCUGAUGACUUCACAGGCUGUGUGAUUGCCUGGAACAGCAACAACCUGGACAGCAAGGUGG | |
| | GAGGCAACUACAACUACCUCUACAGACUGUUCAGGAAGAGCAACCUGAAACCAUUUGAGAGGGACAU | |
| | CAGCACAGAGAUUUACCAGGCUGGCAGCACACCAUGUAAUGGAGUGGAGGGCUUCAACUGUUACUUU | |
| | CCACUCCAAUCCUAUGGCUUCCAACCAACCAAUGGAGUGGGCUACCAACCAUACAGGGUGGUGGUGC | |
| | UGUCCUUUGAACUGCUCCAUGCCCCUGCCACAGUGGAGCCUAAGAGCAGCGACAAGACCCACACAUG | |
| | CCCUCCAUGUCCUGCUCCAGAACUGCUCGGAGGCCCUUCCGUGUUCCUGUUUCCUCCAAAGCCUAAG | |
| | GACACCCUGAUGAUCUCCAGAACACCCGAAGUGACCUGCGUGGUGGUCGAUGUGUCUCACGAGGACC | |
| | CUGAAGUGAAGUUCAAUUGGUACGUCGACGGCGUGGAAGUGCACAACGCCAAGACCAAGCCUAGAGA | |
| | GGAACAGUACAACAGCACCUACAGAGUGGUGUCCGUGCUGACCGUGCUGCACCAGGAUUGGCUGAAC | |
| | GGCAAAGAGUACAAGUGCAAGGUGUCCAACAAGGCCCUGCCAGCUCCUAUCGAGAAAACCAUCAGCA | |
| | AGGCCAAGGGCCAGCCUAGGGAACCCCAGGUUUACACACUGCCUCCAAGCCGGGAAGAGAUGACCAA | |
| | GAACCAGGUGUCCCUGACCUGCCUGGUCAAGGGCUUCUACCCUUCCGAUAUCGCCGUGGAAUGGGAG | |
| | AGCAAUGGCCAGCCUGAGAACAACUACAAGACCACACCUCCUGUGCUGGACAGCGACGGCUCAUUCU | |
| | UCCUGUACAGCAAGCUGACAGUGGACAAGAGCAGAUGGCAGCAGGGCAACGUGUUCAGCUGCAGCGU | |
| | GAUGCACGAGGCCCUGCACAACCACUACACCCAGAAGUCCCUGAGCCUGUCUCCUGGCAAAUGAUAA | |
| | AGACGGGGGGGGCUUAAAUAAUUGAGCCUUAAAGAAGAAAUUCUUUAAGUGGAUGCUCUCAAACUCA | |
| | GGGAAACCUAAAUCUAGUUAUAGACAAGGCAAUCCUGAGCCAAGCCGAAGUAGUAAUUAGUAAGACC | |
| | AGUGGACAAUCGACGGGACCAGUGGACAAUCGACGGAUAACAGCAUAUCUAG | |
| 618 | GGGAGACCCUCGACCGUCGAUUGUCCACUGGUCCCGUCGAUUGUCCACUGGUCAACAAUAGAUGACU | circRBD-UTR- |
| | UACAACUAAUCGGAAGGUGCAGAGACUCGACGGGAGCUACCCUAACGUCAAGACGAGGGUAAAGAGA | 3 |
| | GAGUCCAAUUCUCAAAGCCAAUAGGCAGUAGCGAAAGCUGCAAGAGAAUGAAAACCCCCCCAGAGAA | |
| | UAAACUAGUAUUCUUCUGGUCCCCACAGACUCAGAGAGAACCCCCCACAGCAAGAAUGCCAUCAUCU | |
| | GUCCUCACCCCCAAUUUUCCCUUUUCUUCCCCUGCAACCAUUACGCUUACUCGCAUGUGCAUUGAGU | |
| | GGUGCAUGUGUUGAACAAACAGCUACACUCACAUGGGGGCGGGUUUUCCCGCCCUACGGCCUCUCGC | |
| | GAGGCCCACCCCUUCCCUCCCCUUAUAACUACAGUGCUUUGGUAGGUAAGCAUCCUGAUCCCCCGCG | |
| | GAAGCUGCUCACGUGGCAACUGUGGGGACCCAGACAGGUUAUCAAAGGCACCCGGUCUUUCCGCCUU | |
| | CAGGAGUAUCCCUACUAGUGAAUUCUAGCGGGGCUCUGCUUGGUGCCAACCUCCCCCAAAUGCGCGC | |
| | UGCGGGAGUGCUCUUCCCCAACUCACCCUAGUAUCCUCUCAUGUGUGUGCUUGGUCAGCAUAUCUGA | |
| | GACGAUGUUCCGCUGUCCCAGACCAGUCCAGUAAUGGACGGGCCAGUGCGUGUAGUCGUCUUCCGGC | |
| | UUGUCCGGGGCAUGUUUGGUGAACCGGUGGGGUAAGGUUGGUGUGCCCAACGCCCGUACUUUGGUGA | |
| | CACCUCAAGACCACCCAGGAAUGCCAGGGAGGUACCCCACCUCACGGUGGGAUCUGACCCUGGGCUA | |
| | AUUGUCUACGGUGGUUCUUCUUGCUUCCACUUCUUUCUUCUGUUCACGGCCACCAUGGAUAUGAGAG | |
| | CCCAUGUUCAUCUGCUGGGCCUGCUGCUGCUUUGGCUGCCUGGCGCUAAAUGCAACAUCACCAACCU | |
| | GUGUCCAUUUGGAGAGGUGUUCAAUGCCACCAGGUUUGCCUCUGUCUAUGCCUGGAACAGGAAGAGG | |
| | AUUAGCAACUGUGUGGCUGACUACUCUGUGCUCUACAACUCUGCCUCCUUCAGCACCUUCAAGUGUU | |
| | AUGGAGUGAGCCCAACCAAACUGAAUGACCUGUGUUUCACCAAUGUCUAUGCUGACUCCUUUGUGAU | |
| | UAGGGGAGAUGAGGUGAGACAGAUUGCCCCUGGACAAACAGGCAAGAUUGCUGACUACAACUACAAA | |
| | CUGCCUGAUGACUUCACAGGCUGUGUGAUUGCCUGGAACAGCAACAACCUGGACAGCAAGGUGGGAG | |
| | GCAACUACAACUACCUCUACAGACUGUUCAGGAAGAGCAACCUGAAACCAUUUGAGAGGGACAUCAG | |
| | CACAGAGAUUUACCAGGCUGGCAGCACACCAUGUAAUGGAGUGGAGGGCUUCAACUGUUACUUUCCA | |
| | CUCCAAUCCUAUGGCUUCCAACCAACCAAUGGAGUGGGCUACCAACCAUACAGGGUGGUGGUGCUGU | |
| | CCUUUGAACUGCUCCAUGCCCCUGCCACAGUGGAGCCUAAGAGCAGCGACAAGACCCACACAUGCCC | |
| | UCCAUGUCCUGCUCCAGAACUGCUCGGAGGCCCUUCCGUGUUCCUGUUUCCUCCAAAGCCUAAGGAC | |
| | ACCCUGAUGAUCUCCAGAACACCCGAAGUGACCUGCGUGGUGGUCGAUGUGUCUCACGAGGACCCUG | |
| | AAGUGAAGUUCAAUUGGUACGUCGACGGCGUGGAAGUGCACAACGCCAAGACCAAGCCUAGAGAGGA | |
| | ACAGUACAACAGCACCUACAGAGUGGUGUCCGUGCUGACCGUGCUGCACCAGGAUUGGCUGAACGGC | |
| | AAAGAGUACAAGUGCAAGGUGUCCAACAAGGCCCUGCCAGCUCCUAUCGAGAAAACCAUCAGCAAGG | |
| | CCAAGGGCCAGCCUAGGGAACCCCAGGUUUACACACUGCCUCCAAGCCGGGAAGAGAUGACCAAGAA | |
| | CCAGGUGUCCCUGACCUGCCUGGUCAAGGGCUUCUACCCUUCCGAUAUCGCCGUGGAAUGGGAGAGC | |
| | AAUGGCCAGCCUGAGAACAACUACAAGACCACACCUCCUGUGCUGGACAGCGACGGCUCAUUCUUCC | |
| | UGUACAGCAAGCUGACAGUGGACAAGAGCAGAUGGCAGCAGGGCAACGUGUUCAGCUGCAGCGUGAU | |
| | GCACGAGGCCCUGCACAACCACUACACCCAGAAGUCCCUGAGCCUGUCUCCUGGCAAAUGAUAAAGA | |
| | CGGGGGGGGCUUAAAUAAUUGAGCCUUAAAGAAGAAAUUCUUUAAGUGGAUGCUCUCAAACUCAGGG | |
| | AAACCUAAAUCUAGUUAUAGACAAGGCAAUCCUGAGCCAAGCCGAAGUAGUAAUUAGUAAGACCAGU | |
| | GGACAAUCGACGGGACCAGUGGACAAUCGACGGAUAACAGCAUAUCUAG | |
| 619 | AAAAAAAAAAAACCAAAAAAAAAAAACAAAAAAAAAAAAUAAUUGACUAA | UTR-1 |
| 620 | GGGAAAUAAGAGAGAAAAGAAGAGUAAGAAGAAAUAUAAGACCCCGGCGCCGCCACC | UTR-2 |
| 621 | GAGAAUAAACUAGUAUUCUUCUGGUCCCCACAGACUCAGAGAGAACCCGCCACC | UTR-3 |
| 622 | CAAACACCAUUGUCACACUCCA | hsa-miR-122- |
| | | 5p |
| 623 | CUCCAGCCCCUCCAGGGCUUCCU | hsa-miR-671- |
| | | 5p |
| 624 | AAAUAAGAGAGAAAAGAAGAGUAAGAAGAAAUAUAAGAGCCACCAUGGAGGACGCCAAGAACAUCAA | Luciferase |
| | GAAGGGCCCGGCGCCCUUCUACCCGCUGGAGGACGGGACCGCCGGCGAGCAGCUCCACAAGGCCAUG | mRNA |
| | AAGCGGUACGCCCUGGUGCCGGGCACGAUCGCCUUCACCGACGCCCACAUCGAGGUCGACAUCACCU | |
| | ACGCGGAGUACUUCGAGAUGAGCGUGCGCCUGGCCGAGGCCAUGAAGCGGUACGGCCUGAACACCAA | |
| | CCACCGGAUCGUGGUGUGCUCGGAGAACAGCCUGCAGUUCUUCAUGCCGGUGCUGGGCGCCCUCUUC | |
| | AUCGGCGUGGCCGUCGCCCCGGCGAACGACAUCUACAACGAGCGGGAGCUGCUGAACAGCAUGGGGA | |
| | UCAGCCAGCCGACCGUGGUGUUCGUGAGCAAGAAGGGCCUGCAGAAGAUCCUGAACGUGCAGAAGAA | |
| | GCUGCCCAUCAUCCAGAAGAUCAUCAUCAUGGACAGCAAGACCGACUACCAGGGCUUCCAGUCGAUG | |
| | UACACGUUCGUGACCAGCCACCUCCCGCCGGGCUUCAACGAGUACGACUUCGUCCCGGAGAGCUUCG | |
| | ACCGGGACAAGACCAUCGCCCUGAUCAUGAACAGCAGCGGCAGCACCGGCCUGCCGAAGGGGGUGGC | |
| | CCUGCCGCACCGGACCGCCUGCGUGCGCUUCUCGCACGCCCGGGACCCCAUCUUCGGCAACCAGAUC | |
| | AUCCCGGACACCGCCAUCCUGAGCGUGGUGCCGUUCCACCACGGCUUCGGCAUGUUCACGACCCUGG | |
| | GCUACCUCAUCUGCGGCUUCCGGGUGGUCCUGAUGUACCGGUUCGAGGAGGAGCUGUUCCUGCGGAG | |
| | CCUGCAGGACUACAAGAUCCAGAGCGCGCUGCUCGUGCCGACCCUGUUCAGCUUCUUCGCCAAGAGC | |
| | ACCCUGAUCGACAAGUACGACCUGUCGAACCUGCACGAGAUCGCCAGCGGGGGCGCCCCGCUGAGCA | |
| | AGGAGGUGGGCGAGGCCGUGGCCAAGCGGUUCCACCUCCCGGGCAUCCGCCAGGGCUACGGCCUGAC | |
| | CGAGACCACGAGCGCGAUCCUGAUCACCCCCGAGGGGGACGACAAGCCGGGCGCCGUGGGCAAGGUG | |
| | GUCCCGUUCUUCGAGGCCAAGGUGGUGGACCUGGACACCGGCAAGACCCUGGGCGUGAACCAGCGGG | |
| | GCGAGCUGUGCGUGCGGGGGCCGAUGAUCAUGAGCGGCUACGUGAACAACCCGGAGGCCACCAACGC | |
| | CCUCAUCGACAAGGACGGCUGGCUGCACAGCGGCGACAUCGCCUACUGGGACGAGGACGAGCACUUC | |
| | UUCAUCGUCGACCGGCUGAAGUCGCUGAUCAAGUACAAGGGCUACCAGGUGGCGCCGGCCGAGCUGG | |
| | AGAGCAUCCUGCUCCAGCACCCCAACAUCUUCGACGCCGGCGUGGCCGGGCUGCCGGACGACGACGC | |
| | CGGCGAGCUGCCGGCCGCGGUGGUGGUGCUGGAGCACGGCAAGACCAUGACGGAGAAGGAGAUCGUC | |
| | GACUACGUGGCCAGCCAGGUGACCACCGCCAAGAAGCUGCGGGGCGGCGUGGUGUUCGUGGACGAGG | |
| | UCCCGAAGGGCCUGACCGGGAAGCUCGACGCCCGGAAGAUCCGCGAGAUCCUGAUCAAGGCCAAGAA | |
| | GGGCGGCAAGAUCGCCGUGAGCUAAGCUGCCUUCUGCGGGGCUUGCCUUCUGGCCAUGCCCUUCUUC | |
| | UCUCCCUUGCACCUGUACCUCUUGGUCUUUGAAUAAAGCCUGAGUAGGAAGUAUCCCAAUGGCGCGC | |
| | CGAGCUUGGCAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAA | |

We claim:

1. A circular RNA precursor comprising from 5′ to 3′ direction:
a) a 3′ self-splicing intron fragment; and
b) a 5′ self-splicing intron fragment;
wherein the 3′ self-splicing intron fragment and the 5′ self-splicing intron fragment in combination retain the self-splicing activity of a self-splicing intron and allow generation of a circular RNA through the self-splicing of the circular RNA precursor, and
wherein the self-splicing intron is selected from Group I introns and Group II introns.

2. The circular RNA precursor of claim 1, wherein the self-splicing intron is selected from the following group: cyanobacterium Anabaena Group I Intron such as AnaX, Azoarcus Group I Intron (Azo), Scytalidium dimidiatum Group I Intron (Sd), Staphylococcus phage Twort Group I Intron (Twort), Scytonema-hofmani tRNA fMet group I intron (Sh), or Agrobacterium-tumefaciens group I intron (At), for example, the self-splicing intron has a nucleotide sequence selected from any one of SEQ ID NO: 1-6, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 1-6.

3. The circular RNA precursor of claim 1 or 2, wherein the 3′ self-splicing intron fragment is the 3′ terminal portion of the self-splicing intron starting from an intron internal split site I to the 3′ end of the self-splicing intron, and the 5′ self-splicing intron fragment is the 5′ terminal portion of the self-splicing intron starting from the 5′ end of the self-splicing intron to an intron internal split site II.

4. The circular RNA precursor of any one of claims 1-3, wherein the self-splicing intron is cyanobacterium Anabaena Group I Intron such as AnaX having a nucleotide sequence as set forth in SEQ ID NO. 1, and the intron internal split site II and the intron internal split site I respectively correspond to nucleotide A at position 115 and nucleotide A at position 116 from 5′ end of SEQ ID NO. 1.

5. The circular RNA precursor of any one of claims 1-3, wherein the self-splicing intron is Azoarcus Group I Intron (Azo) which has a nucleotide sequence as set forth in SEQ ID NO. 2, and wherein
1) the intron internal split site II and the intron internal split site I respectively correspond to nucleotide A at position 24 and nucleotide A at position 25 from the 5′ end of SEQ ID NO. 2; or
2) the intron internal split site II and the intron internal split site I respectively correspond to nucleotide A at position 85 and nucleotide A at position 86 from the 5′ end of SEQ ID NO. 2; or
3) the intron internal split site II and the intron internal split site I respectively correspond to nucleotide C at position 71 and nucleotide C at position 72 from the 5′ end of SEQ ID NO. 2; or
4) the intron internal split site II and the intron internal split site I respectively correspond to nucleotide C at position 108 and nucleotide U at position 109 from the 5′ end of SEQ ID NO. 2; or
5) the intron internal split site II and the intron internal split site I respectively correspond to nucleotide A at position 154 and nucleotide A at position 155 from the 5′ end of SEQ ID NO. 2; or
6) the intron internal split site II and the intron internal split site I respectively correspond to nucleotide C at position 198 and nucleotide A at position 199 from the 5′ end of SEQ ID NO. 2.

6. The circular RNA precursor of any one of claims 1-3, wherein the self-splicing intron is Staphylococcus phage Twort Group I Intron (Twort) which has a nucleotide sequence as set forth in SEQ ID NO. 3, and
1) the intron internal split site II and the intron internal split site I respectively correspond to nucleotide U at position 70 and nucleotide A at position 71 from the 5′ end of SEQ ID NO. 3; or
2) the intron internal split site II and the intron internal split site I respectively correspond to nucleotide U at position 103 and nucleotide A at position 104 from the 5′ end of SEQ ID NO. 3; or
3) the intron internal split site II and the intron internal split site I respectively correspond to nucleotide A at position 132 and nucleotide U at position 133 from the 5′ end of SEQ ID NO. 3; or
4) the intron internal split site II and the intron internal split site I respectively correspond to nucleotide U at position 173 and nucleotide C at position 174 from the 5′ end of SEQ ID NO. 3; or
5) the intron internal split site II and the intron internal split site I respectively correspond to nucleotide A at position 206 and nucleotide A at position 207 from the 5′ end of SEQ ID NO. 3; or
6) the intron internal split site II and the intron internal split site I respectively correspond to nucleotide G at position 226 and nucleotide U at position 227 from the 5′ end of SEQ ID NO. 3; or
7) the intron internal split site II and the intron internal split site I respectively correspond to nucleotide U at position 23 and nucleotide A at position 24 from the 5′ end of SEQ ID NO. 3; or
8) the intron internal split site II and the intron internal split site I respectively correspond to nucleotide A at position 82 and nucleotide A at position 83 from the 5′ end of SEQ ID NO. 3.

7. The circular RNA precursor of any one of claims 1-3, wherein the self-splicing intron is Scytalidium dimidiatum Group I Intron (Sd) which has a nucleotide sequence as set forth in SEQ ID NO. 4, and wherein
1) the intron internal split site II and the intron internal split site I respectively correspond to nucleotide A at position 88 and nucleotide G at position 89 from the 5′ end of SEQ ID NO. 4; or
2) the intron internal split site II and the intron internal split site I respectively correspond to nucleotide U at position 173 and nucleotide G at position 174 from the 5′ end of SEQ ID NO. 4; or
3) the intron internal split site II and the intron internal split site I respectively correspond to nucleotide A at position 204 and nucleotide G at position 205 from the 5′ end of SEQ ID NO. 4; or
4) the intron internal split site II and the intron internal split site I respectively correspond to nucleotide C at position 252 and nucleotide G at position 253 from the 5′ end of SEQ ID NO. 4; or
5) the intron internal split site II and the intron internal split site I respectively correspond to nucleotide C at position 370 and nucleotide G at position 371 from the 5′ end of SEQ ID NO. 4.

8. The circular RNA precursor of any one of claims 1-3, wherein the self-splicing intron is Scytonema-hofmani tRNA fMet group I intron (Sh) which has a nucleotide sequence as set forth in SEQ ID NO. 5, and
1) the intron internal split site II and the intron internal split site I respectively correspond to nucleotide A at position 129 and nucleotide A at position 130 from the 5′ end of SEQ ID NO. 5; or
2) the intron internal split site II and the intron internal split site I respectively correspond to nucleotide A at position 60 and nucleotide A at position 61 from the 5′ end of SEQ ID NO. 5; or
3) the intron internal split site II and the intron internal split site I respectively correspond to nucleotide A at position 109 and nucleotide A at position 110 from the 5′ end of SEQ ID NO. 5; or
4) the intron internal split site II and the intron internal split site I respectively correspond to nucleotide A at position 213 and nucleotide A at position 214 from the 5′ end of SEQ ID NO. 5.

9. The circular RNA precursor of any one of claims 1-4, wherein the self-splicing intron is Agrobacterium-tumefaciens group I intron (At), and the intron internal split site II and the intron internal split site I respectively correspond to nucleotide A at position 107 and nucleotide A at position 108 from the 5′end of the At intron having a nucleotide sequence as set forth in SEQ ID NO. 6.

10. The circular RNA precursor of claim 1, wherein the self-splicing intron is the cyanobacterium Anabaena Group I Intron such as AnaX, and the 3′ self-splicing intron fragment comprises or consists of a nucleotide sequence of SEQ ID NO: 7 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7, and the 5′ self-splicing intron fragment comprises or consists of a nucleotide sequence of SEQ ID NO: 8 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 8.

11. The circular RNA precursor of claim 1, wherein the self-splicing intron is the Azoarcus Group I Intron (Azo), and the 3′ self-splicing intron fragment comprises or consists of a nucleotide sequence of SEQ ID NO: 9, 11, 13, 15, 17 or 19 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9, 11, 13, 15, 17 or 19, and the 5′ self-splicing intron fragment comprises or consists of a nucleotide sequence of SEQ ID NO: 10, 12, 14, 16, 18 or 20 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 10, 12, 14, 16, 18 or 20.

12. The circular RNA precursor of claim 1, wherein the self-splicing intron is the Scytalidium dimidiatum Group I Intron (Sd), and the 3′ self-splicing intron fragment comprises or consists of a nucleotide sequence of SEQ ID NO: 37, 39, 41, 43 or 45 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 37, 39, 41, 43 or 45, and the 5′ self-splicing intron fragment comprises or consists of a nucleotide sequence of SEQ ID NO: 38, 40, 42, 44 or 46 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 38, 40, 42, 44 or 46.

13. The circular RNA precursor of claim 1, wherein the self-splicing intron is the Staphylococcus phage Twort Group I Intron (Twort), and the 3′ self-splicing intron fragment comprises or consists of a nucleotide sequence of SEQ ID NO: 21, 23, 25, 27, 29, 31, 33 or 35 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21, 23, 25, 27, 29, 31, 33 or 35, and the 5′ self-splicing intron fragment comprises or consists of a nucleotide sequence of SEQ ID NO: 22, 24, 26, 28, 30, 32, 34 or 36 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 22, 24, 26, 28, 30, 32, 34 or 36.

14. The circular RNA precursor of claim 1, wherein the self-splicing intron is the Scytonema-hofmani tRNA fMet group I intron (Sh), and the 3′ self-splicing intron fragment comprises or consists of a nucleotide sequence of SEQ ID NO: 47, 49, 51 or 53 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 47, 49, 51 or 53 and the 5′ self-splicing intron fragment comprises or consists of a nucleotide sequence of SEQ ID NO: 48, 50, 52 or 54 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 48, 50, 52 or 54.

15. The circular RNA precursor of claim 1, wherein the self-splicing intron is the Agrobacterium-tumefaciens group I intron (At), and the 3′ self-splicing intron fragment comprises or consists of a nucleotide sequence of SEQ ID NO: 55 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 55, and the 5′ self-splicing intron fragment comprises or consists of a nucleotide sequence of SEQ ID NO: 56 or a nucleotide sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 56.

16. The circular RNA precursor of any one of claims 1-15, which further comprises from 5′ to 3′ direction: optionally a first residual circularizing element; a nucleotide sequence of interest; and optionally a second residual circularizing element, located between a) and b), wherein the total length of the first residual circularizing element and the second residual circularizing element is about 0 to about 500 nucleotides; and/or
the circular RNA precursor further comprises a homology arm sequence I upstream of the 3′ self-splicing intron fragment and a homology arm sequence II downstream of the 5′ self-splicing intron fragment.

17. The circular RNA precursor of claim 16, comprising from 5′ to 3′ direction:
i) the 3′ self-splicing intron fragment;
ii) the first residual circularizing element;
iii) the nucleotide sequence of interest;
iv) the second residual circularizing element; and
v) the 5′ self-splicing intron fragment.

18. The circular RNA precursor of claim 16 or 17, wherein the first residual circularizing element comprises or consists of from 5′ to 3′ direction a 3′ exon region and optionally a first spacer, the second residual circularizing element comprises or consists of from 3′ to 5′ direction a 5′ exon region and optionally a second spacer.

19. The circular RNA precursor of claim 18, wherein the 3′ exon region is derived from the native 3′ exon of the self-splicing intron, the 5′ exon region is derived from the native 5′ exon of the self-splicing intron, and the 3′ exon region and 5′ exon region can be recognized and/or spliced by the self-splicing intron or a combination of the 3′ self-splicing intron fragment and the 5′ self-splicing intron fragment.

20. The circular RNA precursor of claim 19, wherein 3′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the native 3′ exon or a contiguous fragment of about 1-about 50 nucleotides starting from the 5′ terminal nucleotide of the native 3′ exon, and
the 5′ exon region is a sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the native 5′ exon or a contiguous fragment of about 1-about 50 nucleotides starting from the 3′ terminal nucleotide of the native 5′ exon.

21. The circular RNA precursor of any one of claims 16-20, wherein the nucleotide sequence of interest comprises at least one protein-coding sequence and a translation initiation element such as an internal ribosome entry site (IRES) operably linked thereto.

22. The circular RNA precursor of claim 21, wherein the translation initiation element is an internal ribosome entry site (IRES), wherein said IRES is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis,
human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is selected from CVB3, HRV-B3, BRAV-1_L, PV1_L, CAV2_L, BRAV-1, PV1, or CAV2.

23. The circular RNA precursor of claim 22, wherein the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 120-526.

24. The circular RNA precursor of any one of claims 21-23, wherein the at least one protein-coding sequence encodes a protein of eukaryotic, prokaryotic or viral origin, for example, a protein for therapeutic or diagnostic use;
for example, the at least one protein-coding sequence encodes IL-2, or an aquaporin, such as AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.

25. The circular RNA precursor of any one of claims 21-24, wherein the at least one protein-coding sequence comprises a nucleotide sequence of any one of SEQ ID NO: 117-118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.

26. The circular RNA precursor of any one of claims 16-23, wherein the nucleotide sequence of interest is a non-coding sequence, for example, the non-coding sequence can be an antisense RNA, aptamer, guide RNA, or non-protein-coding RNA existing in any organism.

27. The circular RNA precursor of any one of claims 1-15 comprising from 5′ to 3′ direction: the 3′ self-splicing intron fragment; a nucleotide sequence of interest; and the 5′ self-splicing intron fragment;
wherein the 3′ self-splicing intron fragment and the 5′ self-splicing intron fragment in combination retain the self-splicing activity of a self-splicing intron and allow generation of a circular RNA which is formed by the nucleotide sequence of interest through the self-splicing of the circular RNA precursor;
wherein the nucleotide sequence of interest comprises from 5′ to 3′ direction: a circularizing sequence fragment II comprising a ribozyme recognition site II at its 5′ end and a circularizing sequence fragment I comprising a ribozyme recognition site I at its 3′ end,
wherein the circularizing sequence fragment I is the 5′ terminal portion of a circularizing sequence, and the circularizing sequence fragment II is the a 3′ terminal portion of a circularizing sequence, and
wherein the ribozyme recognition site I is connected with the ribozyme recognition site II in the generated circular RNA, and thus the circularizing sequence fragment I is connected with the circularizing sequence fragment II to form a circularizing sequence.

28. The circular RNA precursor of claim 27, wherein the circularized nucleotide sequence of interest comprises one or more sequences selected from the following group: at least one protein coding sequences, a non-coding sequence, translation initiation element (TIE), a non-TIE functional element or any combination thereof.

29. The circular RNA precursor of claim 27 or 28, wherein the circularizing sequence is selected from at least one protein-coding sequence, a non-coding sequence, a translation initiation element (TIE), or a non-TIE functional element.

30. The circular RNA precursor of any one of claims 1-15, comprising from 5′ to 3′ direction: the 3′ self-splicing intron fragment; a nucleotide sequence of interest; and the 5′ self-splicing intron fragment,
wherein the 3′ self-splicing intron fragment and the 5′ self-splicing intron fragment in combination retain the self-splicing activity of a self-splicing intron and allow generation of a circular RNA which is formed by the nucleotide sequence of interest through the self-splicing of the circular RNA precursor and which comprises at least one protein-coding sequence;
wherein the nucleotide sequence of interest comprises from 5′ to 3′ direction: a protein-coding sequence fragment II, a translation initiation element such as an internal ribosome entry site (IRES), and a protein-coding sequence fragment I;
wherein the protein-coding sequence fragment I is the 5′ terminal portion of the at least one protein-coding sequence, and the protein-coding sequence fragment II is the 3′ terminal portion of the at least one protein-coding sequence, and
the protein-coding sequence fragment I comprises a ribozyme recognition site I located at its 3′ end, the protein-coding sequence fragment II comprises a ribozyme recognition site II located at its 5′ end, wherein in the generated circular RNA the ribozyme recognition site I is connected with the ribozyme recognition site II, and thus the protein-coding sequence fragment I is connected with the protein-coding sequence fragment II to form the at least one protein-coding sequence.

31. The circular RNA precursor of claim 30, wherein the generated circular RNA comprises at least two protein-coding sequences, and a further translation initiation element, 2A sequence or spacer may be linked between any two adjacent protein-coding sequences, and the translation initiation element comprises: IRES sequence, 5′ UTR sequence, Kozak sequence, a sequence containing m6A, a sequence complementary to ribosome 18S rRNA, or any combination thereof.

32. The circular RNA precursor of any one of claims 29-31, wherein the ribozyme recognition site I and the ribozyme recognition site II are configured such that the circular RNA precursor comprising them has a high circularization efficiency.

33. The circular RNA precursor of any one of claims 29-32, wherein the total length of the ribozyme recognition site I and the ribozyme recognition site II is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

34. The circular RNA precursor of any one of claims 29-33, wherein the total length of the ribozyme recognition site I is about 0 to about 100 nucleotides, about 0 to about 90 nucleotides, about 0 to about 8 nucleotides, about 0 to about 70 nucleotides, about 0 to about 60 nucleotides, about 0 to about 50 nucleotides, about 0 to about 40 nucleotides, about 0 to about 35 nucleotides, about 2 to about 100 nucleotides, about 5 to about 100 nucleotides, about 10 to about 100 nucleotides, about 20 to about 100 nucleotides, about 30 to about 100 nucleotides, or about 30 to about 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length.

35. The circular RNA precursor of any one of claims 29-34, wherein the ribozyme recognition site I and the ribozyme recognition site II are configured to be capable of forming a stem-loop structure, e.g., upon self-splicing for circularization.

36. The circular RNA precursor of claim 35, wherein the loop of the stem-loop structure comprises the splicing junction.

37. The circular RNA precursor of any one of claims 29-36, wherein the ribozyme recognition site I comprises the sequence structure of the following formula: 5′-third non-pairing sequence-third pairing sequence-third loop sequence-3′; and the ribozyme recognition site II comprises the sequence structure of the following formula: 5′-fourth loop sequence-fourth pairing sequence-fourth non-pairing sequence-3′,
wherein the third non-pairing sequence or the fourth non-pairing sequence may be independently present or absent,
the third pairing sequence and the fourth pairing sequence can complementarily pair to each other to form the stem of the stem-loop structure, wherein the third loop sequence and the fourth loop sequence can form the loop of the stem-loop structure, e.g., through self-splicing for circularization.

38. The circular RNA precursor of any one of claims 29-37, wherein the ribozyme recognition site II can be selected from various sequence.

39. The circular RNA precursor of claim 37 or 38, wherein the fourth loop sequence comprises or consists of one or more nucleotides which can pair with the P1 region of the corresponding self-splicing intron to form a P10 duplex region during the circularization.

40. The circular RNA precursor of any one of claims 37-39, wherein the fourth loop sequence consist of a nucleotide sequence of (N)n, wherein N represents any nucleotides (A, G, U, or C), n represents an integer from 1-20, for example, 1, 2, 3, 4, 5, 6, 7, 8, 9, 10, 11, 12, 13, 14, 15, 16, 17, 18, 19, or 20.

41. The circular RNA precursor of any one of claims 37-40, wherein the fourth loop sequence comprises or consists of the sequence of AAAA, AGAA, AA, UUUU, UAAA, CAAA, or GAAA, preferably AAAA.

42. The circular RNA precursor of any one of claims 37-41, wherein the ribozyme recognition site I comprises a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence of any one of SEQ ID NO: 95-116.

43. The circular RNA precursor of any one of claims 37-42, wherein the third loop sequence comprises or consists of one or more nucleotides which can pair with the internal guide sequence (IGS) of the corresponding self-splicing intron to form a P1 duplex region during the circularization.

44. The circular RNA precursor of any one of claims 37-43, wherein the third loop sequence comprises or consists of a nucleotide sequence of any one of SEQ ID NO: 95-116, such as CUU, CAU or CUC, preferably CUU.

45. The circular RNA precursor of any one of claims 37-44, wherein the loop of the stem-loop structure has a sequence of CUUAAAA, CUUUUUU, CUUAA, CUUGAAA, CUUUAAA, CUUAGAA, CUUCAAA or CUCAAAA, preferably CUUAAAA.

46. The circular RNA precursor of any one of claims 29-45, wherein the translation initiation element is an internal ribosome entry site (IRES) which is selected from the following IRES sequences: Taura syndrome virus, blood-sucking bug virus, Tyler's encephalomyelitis virus, simian virus 40, red fire ant virus 1, cereal constriction virus, reticulovirus Endothelial hyperplasia virus, Forman poliovirus 1, soybean inchworm virus, Kashmir bee virus, human rhinovirus 2, glass leafhopper virus-1, human immunodeficiency virus type 1, glass leafhopper virus-1, lice P virus, Hepatitis C virus, Hepatitis A virus, Hepatitis GB virus, Foot and mouth disease virus, Human enterovirus 71, Equine rhinovirus, Tea inchworm-like virus, Encephalomyocarditis virus (EMCV), Drosophila C virus, Cruciferous tobacco Virus, cricket paralysis virus, bovine viral diarrhea virus 1, black queen cell virus, aphid lethal paralysis virus, avian encephalomyelitis virus, acute bee paralysis virus, hibiscus yellow ring spot virus, swine fever virus, human FGF2, human SFTPA1, Human AML1/RUNX1, Drosophila Antennae, Human AQP4, Human AT1R, Human BAG-1, Human BCL2, Human BiP, Human c-IAP1, Human c-myc, Human eIF4G, Mouse NDST4L, Human LEF1, Mouse HIF1α, Human n.myc, mouse Gtx, human p27kipl, human PDGF2/c-sis, human p53, human Pim-1, mouse Rbm3, Drosophila reaper, canine Scamper, Drosophila Ubx, human UNR, mouse UtrA, human VEGF-A, human XIAP, Drosophila hairless, Saccharomyces cerevisiae TFIID, Saccharomyces cerevisiae YAP1, human c-src, human FGF-1, simian picornavirus, turnip crepe disease virus, eIF4G aptamer, Coxsackie Virus B3 (CVB3) or Coxsackie virus A (CVA1/2), preferably, the IRES is selected from CVB3, HRV-B3, BRAV-1_L, PV1_L, CAV2_L, BRAV-1, PV1, or CAV2.

47. The circular RNA precursor of any one of claims 29-46, wherein the IRES comprises a nucleotide sequence set forth in one of SEQ ID NOs: 120-526, or comprises a nucleotide sequence having at least 75%, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 120-526.

48. The circular RNA precursor of any one of claims 29-47, wherein the at least one protein-coding sequence encodes a protein of eukaryotic, prokaryotic or viral origin, for example, a protein for therapeutic or diagnostic use, in particular a protein of mammalian or human origin.

49. The circular RNA precursor of any one of claims 29-48, wherein the at least one protein-coding sequence encodes IL-2, or an aquaporin.

50. The circular RNA precursor of claim 49, wherein the aquaporin is AQP-1 protein or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.

51. The circular RNA precursor of any one of claims 29-50, wherein the at least one protein-coding sequence comprises a nucleotide sequence of any one of SEQ ID NO: 117-118, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 117-118.

52. The circular RNA precursor of any one of claims 29-51, which comprises a nucleotide sequence selected from any one of SEQ ID NO: 530, 531 and 533-534, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 530, 531 and 533-534.

53. A nucleic acid vector for generating a circular RNA molecule, said vector comprises a coding sequence of the circular RNA precursor of any one of claims 1-52.

54. The nucleic acid vector of claim 53, which further comprises an RNA polymerase promoter sequence operably linked to the coding sequence of the circular RNA precursor.

55. The nucleic acid vector of claim 53 or 54, wherein the promoter is a T7 RNA polymerase promoter, a T6 viral RNA polymerase promoter, a SP6 viral RNA polymerase promoter, a T3 viral RNA polymerase promoter or a T4 viral RNA polymerase promoter, preferably a T7 RNA polymerase promoter.

56. A circular RNA, which is prepared from the circular RNA precursor of any one of claims 1-52, preferably said circular RNA has a duration of in vivo expression of at least 24 hours, for example, 36 hours, 48 hours, 72 hours, 96 hours, 5 days, 10 days, 15 days, 16 days, 17 days, 18 days, 19 days, 20 days or more.

57. The circular RNA of claim 56, which comprises or has a nucleotide sequence of any one of SEQ ID NO: 543-558, or a nucleotide sequence having at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NO: 543-558.

58. Use of the circular RNA precursor of any one of claims 1-52 and/or the circular RNA of 56 or 57 as an expression vector.

59. A pharmaceutical composition comprising the circular RNA precursor of any one of claims 1-52 and/or the nucleic acid vector of any one of claims 53-55 and/or the circular RNA of claim 56 or 57, and a pharmaceutically acceptable carrier.

60. A product expressed by the circular RNA of claim 56 or 57.

61. The product of claim 60, which comprises IL-2, POLR2A, or an aquaporin, such as AQP-1 protein having a sequence as set forth in SEQ ID NO: 118 or a protein having the same activity as the AQP-1 protein and derived from the AQP-1 protein through substitution, deletion or addition of 1, 2, 3, 4, 5, 6, 7, 8, 9, 10 or more amino acids in the amino acid sequence of the AQP-1 protein.

62. A method for preventing or treating a disease or condition in a subject, comprising administering to a subject the circular RNA of any one of claims 56-57, or the pharmaceutical composition of claim 59.

63. The method of claim 62, wherein the disease or condition comprises but not limited to xerostomia, xerophthalmia, Sjogren's syndrome or the like.

64. Use of the circular RNA of claim 56 or 57 or the pharmaceutical composition of claim 59 in the manufacture of a medicament for preventing or treating a disease or condition in a subject.

65. The use of claim 64, wherein the disease or condition is xerostomia, xerophthalmia, or Sjogren's syndrome.
